(19) United States
(12) Patent Application Publication
(10) Pub. No.: US 2016/0289290 A1
(43) Pub. Date: Oct. 6, 2016

(54) N-GLYCOSYLATED INSULIN ANALOGUES

(71) Applicant: Merck Sharp & Dohme Corp., Rahway, NJ (US)

(72) Inventors: Michael Meehl, Boston, MA (US); Natarajan Sethuraman, Hanover, NH (US); Sandra Rios, Berkeley Heights, NJ (US)

(21) Appl. No.: 14/995,638

(22) Filed: Jan. 14, 2016

Related U.S. Application Data
(63) Continuation of application No. 14/237,369, filed on Apr. 15, 2014, now abandoned.
(60) Provisional application No. 61/521,142, filed on Aug. 8, 2011.

(51) Int. Cl. C07K 14/62

(52) U.S. Cl. CPC C07K 14/62; A61K 38/00

(57) ABSTRACT

Compositions and formulations comprising N-glycosylated insulin analogues are described. In particular embodiments, the glycosylated insulin analogues are produced in vivo and comprise one or more the N-linked N-glycans selected from high mannose or fucosylated or non-fucosylated hybrid, paucimannose, or complex N-glycans. In other embodiments, the N-glycan comprising the high mannose or fucosylated or non-fucosylated hybrid, paucimannose, or complex N-glycan is attached to the insulin analogue in vitro. Examples of N-glycans include but are not limited to a molecule having a structure selected from N-glycans in the group consisting of Man₍₁₋₉₎GlcNAc₂; or selected from N-glycans in the group consisting of GlcNAc₍₁₋₄₎Man₃GlcNAc₂; or selected from N-glycans in the group consisting of Gal₍₁₋₄₎GlcNAc₍₁₋₄₎Man₃GlcNAc₂; or selected from N-glycans in the group consisting of NANA₍₁₋₄₎Gal₍₁₋₄₎GlcNAc₍₁₋₄₎Man₃GlcNAc₂.

### CROSS REFERENCE TO RELATED APPLICATIONS

This application claims benefit of U.S. Provisional Application No. 61/521,142, which was filed Aug. 8, 2011, and which is incorporated herein in its entirety.

### BACKGROUND OF THE INVENTION

(1) Field of the Invention

The present invention relates to compositions and formulations comprising N-glycosylated insulin analogues. In particular embodiments, the glycosylated insulin analogues are produced in vivo and comprise one or more the N-linked glycans selected from high mannose or fucosylated or non-fucosylated hybrid, paucimannose, or complex N-glycans. In other embodiments, the oligosaccharide or glycan comprising a high mannose or fucosylated or non-fucosylated hybrid, paucimannose, or complex glycan is attached to the insulin analogue in vitro.

(2) Description of Related Art

Insulin is a peptide hormone that is essential for maintaining proper glucose levels in most higher eukaryotes, including humans. Diabetes is a disease in which the individual cannot make insulin or develops insulin resistance. Type I diabetes is a form of diabetes mellitus that results from autoimmune destruction of insulin-producing beta cells of the pancreas. Type II diabetes is a metabolic disorder that is characterized by high blood glucose in the context of insulin resistance and relative insulin deficiency. Left untreated, an individual with Type I or Type II diabetes will die. While not a cure, insulin is effective for lowering glucose in virtually all forms of diabetes. Unfortunately, its pharmacology is not glucose sensitive and as such it is capable of excessive action that can lead to life-threatening hypoglycemia. Inconsistent pharmacology is a hallmark of insulin therapy such that it is extremely difficult to normalize blood glucose without occurrence of hypoglycemia. Furthermore, native insulin is of short duration of action and requires modification to render it suitable for use in control of basal glucose. One central goal in insulin therapy is designing an insulin formulation capable of providing a once a day time action. Mechanisms for extending the action time of an insulin dosage include decreasing the solubility of insulin at the site of injection or covalently attaching sugars, polyethylene glycols, hydrophobic ligands, peptides, or proteins to the insulin.

Molecular approaches to reducing solubility of the insulin have included (1) formulating the insulin as an insoluble suspension with zinc and/or protamine, (2) increasing its isoelectric point through amino acid substitutions and/or additions, such as cationic amino acids to render the molecule insoluble at physiological pH, or (3) covalently modifying the insulin to include a hydrophobic ligand that reduces solubility of the insulin and which binds serum albumin. All of these approaches have been limited by the inherent variability that occurs with precipitation of the molecule at the site of injection, and with the subsequent re-solubilization and transport of the molecule to blood in the form of an active hormone. Even though the resolubilization of the insulin provides a longer duration of action, the insulin is still not responsive to serum glucose levels and the risk of hypoglycemia remains.

Insulin is a two chain heterodimer that is biosynthetically derived from a low potency single chain proinsulin precursor through enzymatic processing. The human insulin analogue consists of two peptide chains, an “A-chain peptide” (SEQ ID NO: 33) and “B-chain peptide” (SEQ ID NO: 25)) bound together by disulfide bonds and having a total of 51 amino acids. The C-terminal region of the B-chain and the two terminal ends of the A-chain associate in a three-dimensional structure that assembles a site for high affinity binding to the insulin receptor. The insulin molecule does not contain N-glycosylation.

Insulin molecules have been modified by linking various moieties to the molecule in an effort to modify the pharmacokinetic or pharmacodynamic properties of the molecule. For example, acylated insulin analogs have been disclosed in a number of publications, which include for example U.S. Pat. Nos. 5,693,609 and 6,011,007. PEGylated insulin analogs have been disclosed in a number of publications including, for example, U.S. Pat. Nos. 5,681,811, 6,309,633; 6,323,311; 6,890,518; 6,890,518; and, 7,585,837. Glycoconjugated insulin analogs have been disclosed in a number of publications including, for example, Internal Publication Nos. WO06082184, WO09089396, WO9010645, U.S. Pat. Nos. 3,847,890; 4,348,387; 7,531,191; and, 7,687,608. Remodeling of peptides, including insulin to include glycan structures for PEGylation and the like have been disclosed in publications including, for example, U.S. Pat. No. 7,138,371 and U.S. Published Application No. 20090053167.

As disclosed herein, applicants provide N-glycosylated insulin and insulin analogues, compositions and formulations comprising the N-glycosylated insulin and insulin analogues, and methods for making the same. These N-glycosylated insulin analogues are active at the insulin receptor and various combinations of N-glycan groups provide the insulin or insulin analogues with various modified pharmacodynamic and/or pharmacokinetic properties.

### BRIEF SUMMARY OF THE INVENTION

The present invention provides glycosylated insulin or insulin analogue molecules, compositions and formulations comprising N-glycosylated insulin and insulin analogues, methods for producing the glycosylated insulin or insulin analogues, and methods for using the glycosylated insulin or insulin analogues. In particular embodiments, the glycosylated insulin or insulin analogue comprises one or more N-glycans, each N-glycan linked to an asparagine residue of a consensus N-linked glycosylation site and is attached to the protein during in vivo expression and processing of the insulin or insulin analogue. In other embodiments, the glycosylated insulin or insulin analogue comprises one or more N-glycans conjugated to an amino acid residue of the molecule in vitro. In further embodiments, the glycosylated insulin or insulin analogue comprises at least two N-glycans, one of which is linked to an asparagine residue comprising an N-linked glycosylation site in vivo and one of which is conjugated to an amino acid residue of the molecule in vitro. The N-glycosylated insulin and insulin analogues (and compositions and formulations comprising the same) are useful for treating Type I and Type II diabetic individuals with a need for an insulin therapy.

Therefore, in particular embodiments, a composition is provided comprising a glycosylated insulin or insulin analogue having an A-chain peptide or functional analogue thereof and a B-chain peptide of insulin or functional analogue thereof, wherein at least one amino acid residue of the A-chain or functional analogue thereof or B-chain amino acid or functional analogue thereof is covalently linked to an N-glycan; the insulin or insulin analogue has three disulfide bonds, and a pharmaceutically acceptable carrier. The first disulfide bond is between the cysteine residues at positions 6 and 11 of the A-chain or functional analogue thereof, the second disulfide bond is between the cysteine residues at position 7 of the A-chain or functional analogue thereof and position 7 of the B-chain or functional analogue thereof, and the third disulfide bond is between the cysteine residues at position 20 of the A-chain or functional analogue thereof and position 19 of the B-chain or functional analogue thereof.

Therefore, in particular embodiments, a composition is provided comprising a glycosylated insulin or insulin analogue having an A-chain peptide comprising the amino acid sequence GIVEQCCTSICSLYQLENYCN (SEQ ID NO: 33); and a B-chain peptide comprising the amino acid sequence HLCGSHLVEALYLVCGERGFF (SEQ ID NO:161), wherein at least one amino acid residue of the A-chain or B-chain amino acid sequence is covalently linked to an N-glycan; and wherein the insulin or insulin analogue optionally further includes up to 17 amino acid substitutions and/or a polypeptide of 3 to 35 amino acids covalently linked to the N-terminus of the A- and/or B-chain peptide, the C-terminus of the A- and/or B-chain peptide, or at the N-terminus to the C-terminus of the B-chain and at the C-terminus to the N-terminus of the A-chain, or combinations thereof; and a pharmaceutically acceptable carrier. The insulin or insulin analogue has three disulfide bonds: the first disulfide bond is between the cysteine residues at positions 6 and 11 of SEQ ID NO:33, the second disulfide bond is between the cysteine residues at position 3 of SEQ ID NO:161 and position 7 of SEQ ID NO:33, and the third disulfide bond is between the cysteine residues at position 15 of SEQ ID NO:161 and position 20 of SEQ ID NO:33.

In further embodiments, the above composition comprises a multiplicity of glycosylated insulin or insulin analogues as recited above; each glycosylated insulin or insulin analogue having at least one N-glycan attached thereto, wherein the predominant or sole N-glycan in the composition consists of a high mannose, hybrid, complex, or paucimannose N-glycan. In a further embodiment, the above composition comprises a plurality of glycosylated insulins or insulin analogues as described above in which a particular high mannose, hybrid, complex, or paucimannose N-glycan species is predominant or the sole N-glycan. For example, the N-glycan species is a molecule having a structure selected from N-glycans in the group consisting of Man₍₁₋₉₎GlcNAc₂; or selected from N-glycans in the group consisting of GlcNAc₍₁₋₄₎Man₃GlcNAc₂; or selected from N-glycans in the group consisting of Gal₍₁₋₄₎GlcNAc₍₁₋₄₎Man₃GlcNAc₂; or selected from N-glycans in the group consisting of NANA₍₁₋₄₎Gal₍₁₋₄₎GlcNAc₍₁₋₄₎Man₃GlcNAc₂. In further embodiments, the predominant or sole N-glycan is selected from the group of N-glycan structures 1 to 106 shown herein.

Further provided are pharmaceutical formulations comprising (a) a multiplicity of N-glycosylated insulin or insulin analogues, each glycosylated insulin or insulin analogue having at least one N-glycan attached thereto, wherein the predominant or sole N-glycan in the formulation consists of a high mannose, hybrid, complex, or paucimannose N-glycan, and (b) a pharmaceutically acceptable carrier. For example, the N-glycan species is a molecule having a structure selected from N-glycans in the group consisting of Man₍₁₋₉₎GlcNAc₂; or selected from N-glycans in the group consisting of GlcNAc₍₁₋₄₎Man₃GlcNAc₂; or selected from N-glycans in the group consisting of Gal₍₁₋₄₎GlcNAc₍₁₋₄₎Man₃GlcNAc₂; or selected from N-glycans in the group consisting of NANA₍₁₋₄₎Gal₍₁₋₄₎GlcNAc₍₁₋₄₎Man₃GlcNAc₂. In further embodiments, the predominant or sole N-glycan is selected from the group of N-glycan structures 1 to 106.

The glycosylated insulin or insulin analogues may be produced in vitro by chemically conjugating the N-glycan to an amino acid residue of the insulin or the glycosylated insulin or insulin analogue can be produced in vivo by (a) providing a host cell capable of producing glycoproteins; (b) introducing into the host cell a nucleic acid molecule encoding an insulin or insulin analogue comprising an N-linked glycosylation site; (c) cultivating the host cell in a medium and under conditions to produce a glycosylated proinsulin or proinsulin analogue precursor or the glycosylated insulin analogue; and (d) recovering the glycosylated proinsulin or proinsulin analogue precursor from the medium and processing the glycosylated proinsulin or proinsulin analogue precursor in vitro to produce the glycosylated insulin or insulin analogue or recovering glycosylated insulin analogue from the medium to produce the glycosylated insulin or insulin analogue. In further aspects, the glycosylated proinsulin or proinsulin analogue precursor is processed in vitro to produce the glycosylated insulin or insulin analogue. Suitable host cells include insect, plant, yeast, or filamentous fungus host cells genetically engineered to produce human-like N-glycans or predominantly particular N-glycan species, for example Pichia pastoris or Saccharomyces cerevisiae genetically engineered to produce human-like N-glycans or predominantly particular N-glycan species.

Further provided is a method for stabilizing an insulin or insulin analogue in a solution or reducing fibrillation of an insulin or insulin analogue in a solution, comprising attaching an N-glycan to an amino acid residue of the insulin or insulin analogue to produce a glycosylated insulin or insulin analogue, wherein the glycosylated insulin or insulin analogue that is attached to the N-glycan is more stable or has reduced fibrillation in the solution than the insulin or insulin analogue not attached to the N-glycan. In particular embodiments, the N-glycan is predominantly or solely a molecule having a structure selected from N-glycans in the group consisting of Man₍₁₋₉₎GlcNAc₂; or selected from N-glycans in the group consisting of GlcNAc₍₁₋₄₎Man₃GlcNAc₂; or selected from N-glycans in the group consisting of Gal₍₁₋₄₎GlcNAc₍₁₋₄₎Man₃GlcNAc₂; or selected from N-glycans in the group consisting of NANA₍₁₋₄₎Gal₍₁₋₄₎GlcNAc₍₁₋₄₎Man₃GlcNAc₂. In further embodiments, the predominant or sole N-glycan is selected from the group of N-glycan structures 1 to 106.

In particular embodiments, the N-glycan is attached to the amino acid residue in vitro by chemically conjugating the N-glycan to an amino acid residue of the insulin or insulin analogue to produce the glycosylated insulin that has increased stability or reduced fibrillation in the solution compared to the insulin or insulin analogue not glycosylated or insulin analogue or the N-glycan is attached to the amino acid residue in vivo to produce the glycosylated insulin or insulin analogue that has increased stability or reduced fibrillation in the solution compared to the insulin or insulin analogue not glycosylated by (a) providing a host cell capable of producing glycoproteins; (b) introducing into the host cell a nucleic acid molecule encoding an insulin or insulin analogue comprising an N-linked glycosylation site; (c) cultivating the host cell in a medium and under conditions to produce a glycosylated proinsulin or proinsulin analogue precursor or the glycosylated insulin analogue; and (d) recovering the glycosylated proinsulin or proinsulin analogue precursor from the medium and processing the glycosylated proinsulin or proinsulin analogue precursor in vitro to produce the glycosylated insulin or insulin analogue or recovering glycosylated insulin analogue from the medium to produce the glycosylated insulin or insulin analogue. In further aspects, the glycosylated proinsulin or proinsulin analogue precursor is processed in vitro to produce the glycosylated insulin or insulin analogue.

In a further embodiment, the N-glycan is attached to the amino acid residue in vivo to produce the glycosylated insulin or insulin analogue by (a) providing a host cell capable of producing glycoproteins; (b) introducing into the host cell a nucleic acid molecule encoding an insulin or insulin analogue in which the nucleic acid molecule encoding the insulin or insulin analogue has been modified to introduce an N-linked glycosylation site into the insulin or insulin analogue encoded therein; (c) cultivating the host cell in a medium and under conditions to produce a glycosylated proinsulin or proinsulin analogue precursor comprising the N-glycan secreted into the medium; (d) recovering the glycosylated proinsulin or proinsulin analogue precursor comprising the N-glycan from the medium; and (e) processing the glycosylated proinsulin or proinsulin analogue precursor in vitro to produce the glycosylated insulin or insulin analogue that has increased stability or reduced fibrillation in the solution compared to the insulin or insulin analogue not glycosylated.

Suitable host cells include insect, plant, yeast, or filamentous fungus host cells genetically engineered to produce human-like N-glycans or predominantly particular N-glycan species, for example Pichia pastoris or Saccharomyces cerevisiae genetically engineered to produce human-like N-glycans or predominantly particular N-glycan species.

Further provided is a composition comprising a glycosylated insulin or insulin analogue having one or more N-glycans wherein the insulin analogue having the one or more N-glycans has increased stability or reduced fibrillation in solution compared to the insulin or insulin analogue not glycosylated and a pharmaceutically acceptable carrier. In a further embodiment, the composition comprises a multiplicity of N-glycosylated insulin or insulin analogues, each glycosylated insulin or insulin analogue having at least one N-glycan attached thereto, wherein the predominant or sole N-glycan in the composition consists of a high mannose, hybrid, complex, or paucimannose N-glycan, and (b) a pharmaceutically acceptable carrier. For example, the N-glycan species is a molecule having a structure selected from N-glycans in the group consisting of Man₍₁₋₉₎GlcNAc₂; or selected from N-glycans in the group consisting of GlcNAc₍₁₋₄₎Man₃GlcNAc₂; or selected from N-glycans in the group consisting of Gal₍₁₋₄₎GlcNAc₍₁₋₄₎Man₃GlcNAc₂; or selected from N-glycans in the group consisting of NANA₍₁₋₄₎Gal₍₁₋₄₎GlcNAc₍₁₋₄₎Man₃GlcNAc₂. In further embodiments, the predominant or sole N-glycan is selected from the group of N-glycan structures 1 to 106. In general, the composition is produced following the in vivo or in vitro methods shown herein.

Further provided is a method for altering a pharmacokinetic or pharmacodynamic property of an insulin or insulin analogue, comprising attaching an N-glycan to an amino acid residue of the insulin or insulin analogue to produce a glycosylated insulin or insulin analogue, wherein the pharmacokinetic or pharmacodynamic property of the glycosylated insulin or insulin analogue that is attached to the N-glycan is altered compared to the insulin or insulin analogue not attached to the N-glycan. In particular embodiments, the N-glycan is predominantly or solely a molecule having a structure selected from N-glycans in the group consisting of Man₍₁₋₉₎GlcNAc₂; or selected from N-glycans in the group consisting of GlcNAc₍₁₋₄₎Man₃GlcNAc₂; or selected from N-glycans in the group consisting of Gal₍₁₋₄₎GlcNAc₍₁₋₄₎Man₃GlcNAc₂; or selected from N-glycans in the group consisting of NANA₍₁₋₄₎Gal₍₁₋₄₎GlcNAc₍₁₋₄₎Man₃GlcNAc₂. In further embodiments, the predominant or sole N-glycan is selected from the group of N-glycan structures 1 to 106.

In particular embodiments, the N-glycan is attached to the amino acid residue in vitro by chemically conjugating the N-glycan to an amino acid residue of the insulin or insulin analogue to produce the glycosylated insulin wherein the pharmacokinetic or pharmacodynamic property of the glycosylated insulin or insulin analogue attached to the N-glycan is altered compared to the insulin or insulin analogue not attached to the N-glycan or insulin analogue or the N-glycan is attached to the amino acid residue in vivo to produce the glycosylated insulin or insulin analogue wherein the pharmacokinetic or pharmacodynamic property of the glycosylated insulin or insulin analogue attached to the N-glycan is altered compared to the insulin or insulin analogue not attached to the N-glycan by ((a) providing a host cell capable of producing glycoproteins; (b) introducing into the host cell a nucleic acid molecule encoding an insulin or insulin analogue comprising an N-linked glycosylation site; (c) cultivating the host cell in a medium and under conditions to produce a glycosylated proinsulin or proinsulin analogue precursor or the glycosylated insulin analogue; and (d) recovering the glycosylated proinsulin or proinsulin analogue precursor from the medium and processing the glycosylated proinsulin or proinsulin analogue precursor in vitro to produce the glycosylated insulin or insulin analogue or recovering glycosylated insulin analogue from the medium to produce the glycosylated insulin or insulin analogue. In further aspects, the glycosylated proinsulin or proinsulin analogue precursor is processed in vitro to produce the glycosylated insulin or insulin analogue.

In a further embodiment, the N-glycan is attached to the amino acid residue in vivo to produce the glycosylated insulin or insulin analogue by (a) providing a host cell capable of producing glycoproteins; (b) introducing into the host cell a nucleic acid molecule encoding an insulin or insulin analogue in which the nucleic acid molecule encoding the insulin or insulin analogue has been modified to introduce an N-linked glycosylation site into the insulin or insulin analogue encoded therein; (c) cultivating the host cell in a medium and under conditions to produce a glycosylated proinsulin or proinsulin analogue precursor comprising the N-glycan secreted into the medium; (d) recovering the glycosylated proinsulin or proinsulin analogue precursor comprising the N-glycan from the medium; and (e) processing the glycosylated proinsulin or proinsulin analogue precursor in vitro to produce the glycosylated insulin or insulin analogue wherein the pharmacokinetic or pharmacodynamic property of the glycosylated insulin or insulin analogue attached to the N-glycan is altered compared to the insulin or insulin analogue not attached to the N-glycan.

Suitable host cells include insect, plant, yeast, or filamentous fungus host cells genetically engineered to produce human-like N-glycans or predominantly particular N-glycan species, for example Pichia pastoris or Saccharomyces cerevisiae genetically engineered to produce human-like N-glycans or predominantly particular N-glycan species.

Further provided is a composition comprising a glycosylated insulin or insulin analogue having one or more N-glycans wherein the insulin analogue having the one or more N-glycans has a pharmacokinetic or pharmacodynamic property that is altered compared to the insulin or insulin analogue not attached to the one or more N-glycans and a pharmaceutically acceptable carrier. In a further embodiment, the composition comprises a multiplicity of N-glycosylated insulin or insulin analogues, each glycosylated insulin or insulin analogue having at least one N-glycan attached thereto, wherein the predominant or sole N-glycan in the composition consists of a high mannose, hybrid, complex, or paucimannose N-glycan, and (b) a pharmaceutically acceptable carrier. For example, the N-glycan species is a molecule having a structure selected from N-glycans in the group consisting of Man₍₁₋₉₎GlcNAc₂; or selected from N-glycans in the group consisting of GlcNAc₍₁₋₄₎Man₃GlcNAc₂; or selected from N-glycans in the group consisting of Gal₍₁₋₄₎GlcNAc₍₁₋₄₎Man₃GlcNAc₂; or selected from N-glycans in the group consisting of NANA₍₁₋₄₎Gal₍₁₋₄₎GlcNAc₍₁₋₄₎Man₃GlcNAc₂. In further embodiments, the predominant or sole N-glycan is selected from the group of N-glycan structures 1 to 106. In general, the composition is produced following the in vivo or in vitro methods shown herein.

Further provided is a method for producing an insulin or insulin analogue that preferentially targets a receptor in the liver, comprising attaching an N-glycan comprising a terminal galactose residue to an amino acid residue of the insulin or insulin analogue to produce a glycosylated insulin or insulin analogue, wherein the glycosylated insulin or insulin analogue attached to the N-glycan preferentially targets a receptor in the liver. In particular embodiments, the N-glycan is predominantly or solely a molecule having a structure selected from N-glycans in the group consisting of Man₍₁₋₉₎GlcNAc₂; or selected from N-glycans in the group consisting of GlcNAc₍₁₋₄₎Man₃GlcNAc₂; or selected from N-glycans in the group consisting of Gal₍₁₋₄₎GlcNAc₍₁₋₄₎Man₃GlcNAc₂; or selected from N-glycans in the group consisting of NANA₍₁₋₄₎Gal₍₁₋₄₎GlcNAc₍₁₋₄₎Man₃GlcNAc₂. In further embodiments, the predominant or sole N-glycan is selected from the group of N-glycan structures 1 to 106.

In particular embodiments, the N-glycan is attached to the amino acid residue in vitro by chemically conjugating the N-glycan to an amino acid residue of the insulin or insulin analogue to produce the glycosylated insulin that preferentially targets the liver receptor or the N-glycan is attached to the amino acid residue in vivo to produce the glycosylated insulin or insulin analogue that preferentially targets the liver receptor by (a) providing a host cell capable of producing glycoproteins; (b) introducing into the host cell a nucleic acid molecule encoding an insulin or insulin analogue comprising an N-linked glycosylation site; (c) cultivating the host cell in a medium and under conditions to produce a glycosylated proinsulin or proinsulin analogue precursor or the glycosylated insulin analogue; and (d) recovering the glycosylated proinsulin or proinsulin analogue precursor from the medium and processing the glycosylated proinsulin or proinsulin analogue precursor in vitro to produce the glycosylated insulin or insulin analogue or recovering glycosylated insulin analogue from the medium to produce the glycosylated insulin or insulin analogue. In further aspects, the glycosylated proinsulin or proinsulin analogue precursor is processed in vitro to produce the glycosylated insulin or insulin analogue.

In a further embodiment, the N-glycan is attached to the amino acid residue in vivo to produce the glycosylated insulin or insulin analogue by (a) providing a host cell capable of producing glycoproteins; (b) introducing into the host cell a nucleic acid molecule encoding an insulin or insulin analogue in which the nucleic acid molecule encoding the insulin or insulin analogue has been modified to introduce an N-linked glycosylation site into the insulin or insulin analogue encoded therein; (c) cultivating the host cell in a medium and under conditions to produce a glycosylated proinsulin or proinsulin analogue precursor comprising the N-glycan secreted into the medium; (d) recovering the glycosylated proinsulin or proinsulin analogue precursor comprising the N-glycan from the medium; and (e) processing the glycosylated proinsulin or proinsulin analogue precursor in vitro to produce the glycosylated insulin or insulin analogue that preferentially targets the liver receptor. In a further embodiment, the N-glycan consists of a fucosylated or non-fucosylated glycan having a GalGlcNAcMan₅GlcNAc₂ structure or a structure selected from the group consisting of Gal₍₁₋₄₎GlcNAc₍₁₋₄₎Man₃GlcNAc₂ structures.

Suitable host cells include insect, plant, yeast, or filamentous fungus host cells genetically engineered to produce human-like N-glycans or predominantly particular N-glycan species, for example Pichia pastoris or Saccharomyces cerevisiae genetically engineered to produce human-like N-glycans or predominantly particular N-glycan species.

Further provided is a composition comprising a glycosylated insulin or insulin analogue having one or more N-glycans wherein the insulin analogue having the one or more N-glycans preferentially targets a receptor in the liver and a pharmaceutically acceptable carrier. In a further embodiment, the composition comprises a multiplicity of N-glycosylated insulin or insulin analogues, each glycosylated insulin or insulin analogue having at least one N-glycan attached thereto, wherein the predominant or sole N-glycan in the composition consists of a high mannose, hybrid, complex, or paucimannose N-glycan, and (b) a pharmaceutically acceptable carrier. For example, the N-glycan species is a molecule having a structure selected from N-glycans in the group consisting of Man₍₁₋₉₎GlcNAc₂; or selected from N-glycans in the group consisting of GlcNAc₍₁₋₄₎Man₃GlcNAc₂; or selected from N-glycans in the group consisting of Gal₍₁₋₄₎GlcNAc₍₁₋₄₎Man₃GlcNAc₂; or selected from N-glycans in the group consisting of NANA₍₁₋₄₎Gal₍₁₋₄₎GlcNAc₍₁₋₄₎Man₃GlcNAc₂. In further embodiments, the predominant or sole N-glycan is selected from the group of N-glycan structures 1 to 106. In general, the composition is produced following the in vivo or in vitro methods shown herein.

Further provided is a method for producing an insulin or insulin analogue that has at least one pharmacokinetic or pharmacodynamic property of the conjugate sensitive to serum concentration of glucose when used in a treatment for diabetes, comprising conjugating an N-glycan to an amino acid residue of the insulin or insulin analogue to produce a glycosylated insulin or insulin analogue, wherein the glycosylated insulin or insulin analogue that is attached to the N-glycan has at least one pharmacokinetic or pharmacodynamic property sensitive to serum concentration of glucose. In particular embodiments, the N-glycan is predominantly or solely a molecule having a structure selected from N-glycans in the group consisting of Man₍₁₋₉₎GlcNAc₂; or selected from N-glycans in the group consisting of GlcNAc₍₁₋₄₎Man₃GlcNAc₂; or selected from N-glycans in the group consisting of Gal₍₁₋₄₎GlcNAc₍₁₋₄₎Man₃GlcNAc₂; or selected from N-glycans in the group consisting of NANA₍₁₋₄₎Gal₍₁₋₄₎GlcNAc₍₁₋₄₎Man₃GlcNAc₂. In further embodiments, the predominant or sole N-glycan is selected from the group of N-glycan structures 1 to 106.

In particular embodiments, the N-glycan is attached to the amino acid residue in vitro by chemically conjugating the N-glycan to an amino acid residue of the insulin or insulin analogue to produce the glycosylated insulin that has at least one pharmacokinetic or pharmacodynamic property sensitive to serum concentration of glucose or the N-glycan is attached to the amino acid residue in vivo to produce the glycosylated insulin or insulin analogue that has at least one pharmacokinetic or pharmacodynamic property sensitive to serum concentration of glucose by (a) providing a host cell capable of producing glycoproteins; (b) introducing into the host cell a nucleic acid molecule encoding an insulin or insulin analogue comprising an N-linked glycosylation site; (c) cultivating the host cell in a medium and under conditions to produce a glycosylated proinsulin or proinsulin analogue precursor or the glycosylated insulin analogue; and (d) recovering the glycosylated proinsulin or proinsulin analogue precursor from the medium and processing the glycosylated proinsulin or proinsulin analogue precursor in vitro to produce the glycosylated insulin or insulin analogue or recovering glycosylated insulin analogue from the medium to produce the glycosylated insulin or insulin analogue. In further aspects, the glycosylated proinsulin or proinsulin analogue precursor is processed in vitro to produce the glycosylated insulin or insulin analogue.

In a further embodiment, the N-glycan is attached to the amino acid residue in vivo to produce the glycosylated insulin or insulin analogue by (a) providing a host cell capable of producing glycoproteins; (b) introducing into the host cell a nucleic acid molecule encoding an insulin or insulin analogue in which the nucleic acid molecule encoding the insulin or insulin analogue has been modified to introduce an N-linked glycosylation site into the insulin or insulin analogue encoded therein; (c) cultivating the host cell in a medium and under conditions to produce a glycosylated proinsulin or proinsulin analogue precursor comprising the N-glycan secreted into the medium; (d) recovering the glycosylated proinsulin or proinsulin analogue precursor comprising the N-glycan from the medium; and (e) processing the glycosylated proinsulin or proinsulin analogue precursor in vitro to produce the glycosylated insulin or insulin analogue that has at least one pharmacokinetic or pharmacodynamic property sensitive to serum concentration of glucose.

Suitable host cells include insect, plant, yeast, or filamentous fungus host cells genetically engineered to produce human-like N-glycans or predominantly particular N-glycan species, for example Pichia pastoris or Saccharomyces cerevisiae genetically engineered to produce human-like N-glycans or predominantly particular N-glycan species.

Further provided is composition comprising a glycosylated insulin or insulin analogue having one or more N-glycans wherein the one or more N-glycans renders at least one pharmacokinetic or pharmacodynamic property of the insulin or insulin analogue having the one or more N-glycans sensitive to serum concentration of glucose when used in a treatment for diabetes and a pharmaceutically acceptable carrier. In a further embodiment, the composition comprises a multiplicity of N-glycosylated insulin or insulin analogues, each glycosylated insulin or insulin analogue having at least one N-glycan attached thereto, wherein the predominant or sole N-glycan in the composition consists of a high mannose, hybrid, complex, or paucimannose N-glycan, and (b) a pharmaceutically acceptable carrier. For example, the N-glycan species is a molecule having a structure selected from N-glycans in the group consisting of Man₍₁₋₉₎GlcNAc₂; or selected from N-glycans in the group consisting of GlcNAc₍₁₋₄₎Man₃GlcNAc₂; or selected from N-glycans in the group consisting of Gal₍₁₋₄₎GlcNAc₍₁₋₄₎Man₃GlcNAc₂; or selected from N-glycans in the group consisting of NANA₍₁₋₄₎Gal₍₁₋₄₎GlcNAc₍₁₋₄₎Man₃GlcNAc₂. In further embodiments, the predominant or sole N-glycan is selected from the group of N-glycan structures 1 to 106. In general, the composition is produced following the in vivo or in vitro methods shown herein.

In particular aspects of any of the above embodiments, the N-glycan is covalently linked to the amide group of an Asn residue in a β1 linkage. In further embodiments, the Asn residue is at amino acid position 10 or 21 of the native A-chain peptide or amino acid position 3, 25, or 28 of the native B-chain peptide with the proviso that if the Asn is at the 3 position of the B-chain then the amino acid at position 5 of the B-chain peptide is a Ser or Thr and if the Asn is at position 21 of the A-chain then the A-chain peptide further includes at the C-terminus of the Asn a dipeptide of amino acid sequence Xaa-Ser or Xaa-Thr wherein Xaa is any amino acid except Pro. In further embodiments, the Asn is at position 21 of the A-chain peptide and the A-chain peptide further includes at the C-terminus of the Asn a dipeptide of amino acid sequence Xaa-Ser or Xaa-Thr wherein Xaa is any amino acid except Pro. In particular embodiments, the Xaa is Lys, Arg, or Gly.

In further aspects of any of the above embodiments, a tripeptide having the amino acid sequence Asn-Xaa-Ser or Asn-Xaa-Thr wherein Xaa is any amino acid except Pro is covalently linked to the N-terminus of the A-chain in a peptide bond. In particular embodiments, the Xaa is Thr.

In further aspects of any of the above embodiments, a tripeptide having the amino acid sequence Asn-Xaa-Ser or Asn-Xaa-Thr wherein Xaa is any amino acid except Pro is covalently linked to the N-terminus of the B-chain in a peptide bond. In particular embodiments, the Xaa is Thr.

In further aspects of any of the above embodiments, a tripeptide having the amino acid sequence Asn-Xaa-Ser or Asn-Xaa-Thr wherein Xaa is any amino acid except Pro is covalently linked to the C-terminus of the B-chain in a peptide bond.

In further aspects of any of the above embodiments, the N-terminus of the A-chain peptide, the N-terminus of the B-chain peptide, the epsilon-amino group of Lys at position 29 of the B-chain peptide, or any other available amino group is covalently linked to a C₁₋₂₀ alkyl group.

In further aspects of any of the above embodiments, the N-glycan is attached to the insulin or insulin molecule at an amino acid residue at the N- or C-terminus of the A-chain peptide or B-chain peptide.

In further aspects of any of the above embodiments, the N-glycan is attached to the insulin or insulin molecule at a histidine, cysteine, or lysine residue.

In further aspects of any of the above embodiments, the insulin or insulin analogue is a heterodimer molecule comprising an A-chain peptide and a B-chain peptide wherein the A-chain peptide is covalently linked to the B-chain by two disulfide bonds or a single-chain molecule comprising an A-chain peptide connected to the B-chain peptide by a connecting peptide wherein the A-chain and the B-chain are covalently linked by two disulfide bonds.

In further aspects of any of the above embodiments, one or more amino acids at positions 1 to 4 and/or 26 to 30 of the B-chain peptide have been deleted.

In further aspects of any of the above embodiments, the amino acids substitutions are selected from positions 5, 8, 9, 10, 12, 14, 15, 17, 18, and 21 of the A-chain peptide and positions 1, 2, 3, 4, 5, 9, 10, 13, 14, 17, 20, 21, 22, 23, 26, 27, 28, 29, and 30 of the B-chain peptide.

In further aspects of any of the above embodiments, the amino acid at position 21 of the A-chain peptide is Gly and the B-chain includes the dipeptide Arg-Arg is covalently linked to the Thr at the position 30 of the B-chain peptide.

In further aspects of any of the above embodiments, the B-chain peptide lacks a threonine residue at position 30.

In particular aspects of any of the above embodiments, compositions of the glycosylated insulin or insulin analogues are provided wherein the N-glycans in the compositions are high mannose N-glycans, fucosylated or non-fucosylated hybrid N-glycans, paucimannose N-glycans, complex N-glycans, including bisected or multiantennary N-glycans, or combinations thereof. Exemplary N-glycans include but are not limited to a fucosylated or non-fucosylated N-glycans having a structure selected from the group consisting of GlcNAc₍₁₋₄₎Man₃GlcNAc₂; Gal₍₁₋₄₎GlcNAc₍₁₋₄₎Man₃GlcNAc₂; and NANA₍₁₋₄₎Gal₍₁₋₄₎GlcNAc₍₁₋₄₎Man₃GlcNAc₂ wherein the integer indicates the number of saccharide residues. In general, the glycosylated insulin or insulin analogue may have at least 20% of the activity of native insulin at the insulin receptor. In particular embodiments, the glycosylated insulin or insulin analogue may at least 50%, 60%, 70%, 80%, or 90% of the activity of native insulin at the insulin receptor. In further aspects, at least 70%, 75%, 80%, 85%, 90%, 95%, 96%, 97%, 98%, 99%, or 100% of the insulin or insulin analogues in the composition are glycosylated.

In particular aspects of any of the above embodiments, the glycosylated insulin or analogue compositions provided herein comprise glycosylated insulin or insulin analogues having at least one hybrid N-glycan selected from the group consisting of GlcNAcMan₃GlcNAc₂; GalGlcNAcMan₃GlcNAc₂; NANAGalGlcNAcMan₃GlcNAc₂; GlcNAcMan₅GlcNAc₂; GalGlcNAcMan₅GlcNAc₂; and NANAGalGlcNAcMan₅GlcNAc₂ wherein the integer indicates the number of saccharide residues.

In particular aspects, the hybrid N-glycan is the predominant N-glycan species in the composition. In further aspects, the hybrid N-glycan is a particular N-glycan species that comprises about 30 mole %, 40 mole %, 50 mole %, 60 mole %, 70 mole %, 80 mole %, 90 mole %, 95 mole %, 97 mole %, 98 mole %, 99 mole %, or 100 mole % of the N-glycans in the composition. In particular embodiments in which the hybrid N-glycan comprises a NANA residue, the NANA is linked to the galactose residue in an α2,6 linkage or the NANA is linked to the galactose residue in an α2,3 linkage.

In further aspects, at least 70%, 75%, 80%, 85%, 90%, 95%, 96%, 97%, 98%, 99%, or 100% of the insulin or insulin analogues in the composition are glycosylated. In further aspects, at least 70%, 75%, 80%, 85%, 90%, 95%, 96%, 97%, 98%, 99%, or 100% of the insulin or insulin analogues in the composition include the N-glycan.

In particular aspects of any of the above embodiments, the glycosylated insulin or insulin analogue compositions provided herein comprise glycosylated insulin or insulin analogues having at least one complex N-glycan selected from the group consisting of GlcNAc₂Man₃GlcNAc₂; GalGlcNAc₂Man₃GlcNAc₂; Gal₂GlcNAc₂Man₃GlcNAc₂; NANAGal₂GlcNAc₂Man₃GlcNAc₂; and NANA₂Gal₂GlcNAc₂Man₃GlcNAc₂ wherein the integer indicates the number of saccharide residues.

In particular aspects, the complex N-glycan is the predominant N-glycan species in the composition. In further aspects, the complex N-glycan is a particular N-glycan species that comprises about 30 mole %, 40 mole %, 50 mole %, 60 mole %, 70 mole %, 80 mole %, 90 mole %, 95 mole %, 97 mole %, 98 mole %, 99 mole %, or 100 mole % of the N-glycans in the composition.

In further aspects, at least 70%, 75%, 80%, 85%, 90%, 95%, 96%, 97%, 98%, 99%, or 100% of the insulin or insulin analogues in the composition are glycosylated. In further aspects, at least 70%, 75%, 80%, 85%, 90%, 95%, 96%, 97%, 98%, 99%, or 100% of the insulin or insulin analogues in the composition include the N-glycan. In particular embodiments in which the complex N-glycan comprises a NANA residue, the NANA is linked to the galactose residue in an α2,6 linkage or the NANA is linked to the galactose residue in an α2,3 linkage.

In particular aspects of any of the above embodiments, the N-glycan is fusosylated. In general, the fucose is in an α1,3-linkage with the GlcNAc at the reducing end of the N-glycan, an α1,6-linkage with the GlcNAc at the reducing end of the N-glycan, an α1,2-linkage with the Gal (galactose) at the non-reducing end of the N-glycan or adjacent to the saccharide at the non-reducing end of the N-glycan, an α1,3-linkage or α1,4-linkage with the GlcNAc at the non-reducing end of the N-glycan or near the non-reducing end of the N-glycan.

In particular aspects of any of the above embodiments, the glycoform is in an α1,3-linkage or α1,6-linkage fucose to produce a glycoform selected from the group consisting of GlcNAcMan₅GlcNAc₂(Fuc), GalGlcNAcMan₅GlcNAc₂(Fuc), NANAGalGlcNAcMan₅GlcNAc₂(Fuc), Man₅GlcNAc₂(Fuc), Man₃GlcNAc₂(Fuc), GlcNAcMan₃GlcNAc₂(Fuc), GlcNAc₂Man₃GlcNAc₂(Fuc), GalGlcNAc₂Man₃GlcNAc₂(Fuc), Gal₂GlcNAc₂Man₃GlcNAc₂(Fuc), NANAGal₂GlcNAc₂Man₃GlcNAc₂(Fuc), and NANA₂Gal₂GlcNAc₂Man₃GlcNAc₂(Fuc); in an α1,3-linkage or α1,4-linkage fucose to produce a glycoform selected from the group consisting of GlcNAc(Fuc)Man₅GlcNAc₂, GalGlcNAc(Fuc)Man₅GlcNAc₂, NANAGalGlcNAc(Fuc)Man₅GlcNAc₂, GlcNAc(Fuc)Man₃GlcNAc₂, GlcNAc₂(Fuc₁₋₂₎Man₃GlcNAc₂, GalGlcNAc₂(Fuc₁₋₂₎Man₃GlcNAc₂, Gal₂GlcNAc₂(Fuc₁₋₂₎Man₃GlcNAc₂, NANAGal₂GlcNAc₂(Fuc₁₋₂₎Man₃GlcNAc₂, and NANA₂Gal₂GlcNAc₂(Fuc₁₋₂₎Man₃GlcNAc₂; or in an α1,2-linkage fucose to produce a glycoform selected from the group consisting of Gal(Fuc)GlcNAc₂Man₃GlcNAc₂, Gal₂(Fuc₁₋₂)GlcNAc₂Man₃GlcNAc₂, NANAGal₂(Fuc₁₋₂)GlcNAc₂Man₃GlcNAc₂, and NANA₂Gal₂(Fuc₁₋₂)GlcNAc₂Man₃GlcNAc₂ wherein the integer indicates the number of saccharide residues.

In particular aspects, the fucosylated N-glycan is the predominant N-glycan species in the composition. In further aspects, the predominant fucosylated N-glycan is a particular N-glycan species that comprises about 30 mole %, 40 mole %, 50 mole %, 60 mole %, 70 mole %, 80 mole %, 90 mole %, 95 mole %, 97 mole %, 98 mole %, 99 mole %, or 100 mole % of the N-glycans in the composition.

In further aspects, at least 70%, 75%, 80%, 85%, 90%, 95%, 96%, 97%, 98%, 99%, or 100% of the insulin or insulin analogues in the composition include the N-glycan. In further aspects, at least 70%, 75%, 80%, 85%, 90%, 95%, 96%, 97%, 98%, 99%, or 100% of the insulin or insulin analogues in the composition are glycosylated. In particular embodiments in which the fucosylated N-glycan comprises a NANA residue, the NANA is linked to the galactose residue in an α2,6 linkage or the NANA is linked to the galactose residue in an α2,3 linkage.

In particular aspects of any of the above embodiments, the complex N-glycans further include fucosylated and non-fucosylated multiantennary N-glycan species. In particular aspects, the fucosylated or non-fucosylated multiantennary N-glycan is the predominant N-glycan species in the composition.

In further aspects, the predominant fucosylated or non-fucosylated multiantennary N-glycan is a particular N-glycan species that comprises about 30 mole %, 40 mole %, 50 mole %, 60 mole %, 70 mole %, 80 mole %, 90 mole %, 95 mole %, 97 mole %, 98 mole %, 99 mole %, or 100 mole % of the N-glycans in the composition. In further aspects, at least 70%, 75%, 80%, 85%, 90%, 95%, 96%, 97%, 98%, 99%, or 100% of the insulin or insulin analogues in the composition are glycosylated. In further aspects, at least 70%, 75%, 80%, 85%, 90%, 95%, 96%, 97%, 98%, 99%, or 100% of the insulin or insulin analogues in the composition include the N-glycan.

In particular aspects of any of the above embodiments, the complex N-glycans further include bisected N-glycan species. In particular aspects, the bisected N-glycan is the predominant N-glycan species in the composition. In further aspects, the predominant bisected N-glycan is a particular N-glycan species that comprises about 30 mole %, 40 mole %, 50 mole %, 60 mole %, 70 mole %, 80 mole %, 90 mole %, 95 mole %, 97 mole %, 98 mole %, 99 mole %, or 100 mole % of the N-glycans in the composition. In further aspects, at least 70%, 75%, 80%, 85%, 90%, 95%, 96%, 97%, 98%, 99%, or 100% of the insulin or insulin analogues in the composition are glycosylated. In further aspects, at least 70%, 75%, 80%, 85%, 90%, 95%, 96%, 97%, 98%, 99%, or 100% of the insulin or insulin analogues in the composition include the N-glycan.

In particular aspects of any of the above embodiments, the glycosylated insulin or insulin analogues consist of high a mannose N-glycan selected from Man₅GlcNAc₂, Man₆GlcNAc₂, Man₇GlcNAc₂, Man₈GlcNAc₂, Man₉GlcNAc₂, or N-glycans that consist of the Man₃GlcNAc₂ N-glycan structure wherein the integer indicates the number of saccharide residues.

In particular aspects, the N-glycan is the predominant N-glycan species in the composition. In further aspects, the predominant N-glycan is a particular N-glycan species that comprises about 30 mole %, 40 mole %, 50 mole %, 60 mole %, 70 mole %, 80 mole %, 90 mole %, 95 mole %, 97 mole %, 98 mole %, 99 mole %, or 100 mole % of the N-glycans in the composition. In further aspects, at least 70%, 75%, 80%, 85%, 90%, 95%, 96%, 97%, 98%, 99%, or 100% of the insulin or insulin analogues in the composition are glycosylated. In further aspects, at least 70%, 75%, 80%, 85%, 90%, 95%, 96%, 97%, 98%, 99%, or 100% of the insulin or insulin analogues in the composition include the N-glycan.

In particular aspects of any of the above embodiments, the N-glycan may be Man₄GlcNAc₂ or an N-glycan consisting of a ManGlcNAc₂ or GlcNAcManGlcNAc₂ structure. In particular aspects, the N-glycan is the predominant N-glycan species in the composition. In further aspects, the predominant N-glycan is a particular N-glycan species that comprises about 30 mole %, 40 mole %, 50 mole %, 60 mole %, 70 mole %, 80 mole %, 90 mole %, 95 mole %, 97 mole %, 98 mole %, 99 mole %, or 100 mole % of the N-glycans in the composition. In further aspects, at least 70%, 75%, 80%, 85%, 90%, 95%, 96%, 97%, 98%, 99%, or 100% of the insulin or insulin analogues in the composition are glycosylated. In further aspects, at least 70%, 75%, 80%, 85%, 90%, 95%, 96%, 97%, 98%, 99%, or 100% of the insulin or insulin analogues in the composition include the N-glycan.

The glycosylated insulin or insulin analogues comprising the present invention exclude embodiments wherein the N-glycan attached thereto is a hypermannosylated N-glycan or an N-glycan that includes one or more mannose residues linked to another mannose residue in a β linkage.

Further provided is the use of an N-glycosylated insulin or insulin analogue for the preparation of a composition or formulation for the treatment of diabetes. Further provided is a composition as disclosed herein for the treatment of diabetes. For example, a glycosylated insulin or insulin analogue having an A-chain peptide comprising the amino acid sequence GIVEQCCTSICSLYQLENYCN (SEQ ID NO: 33); and a B-chain peptide comprising the amino acid sequence HLCGSHLVEALYLVCGERGFF (SEQ ID NO:161), wherein at least one amino acid residue of the A-chain or B-chain amino acid sequence is covalently linked to an N-glycan; and wherein the insulin or insulin analogue optionally further includes up to 17 amino acid substitutions and/or a polypeptide of 3 to 35 amino acids covalently linked to N-terminus, C-terminus, or which is covalently linked at the N-terminus to the C-terminus of the B-chain and at the C-terminus to the N-terminus of the A-chain; and a pharmaceutically acceptable carrier for the treatment of diabetes.

### DEFINITIONS

As used herein, the term “insulin” means the active principle of the pancreas that affects the metabolism of carbohydrates in the animal body and which is of value in the treatment of diabetes mellitus. The term includes synthetic and biotechnologically derived products that are the same as, or similar to, naturally occurring insulins in structure, use, and intended effect and are of value in the treatment of diabetes mellitus.

The term “insulin” or “insulin molecule” is a generic term that designates the 51 amino acid heterodimer comprising the A-chain peptide having the amino acid sequence shown in SEQ ID NO: 33 and the B-chain peptide having the amino acid sequence shown in SEQ ID NO: 25, wherein the cysteine residues a positions 6 and 11 of the A chain are linked in a disulfide bond, the cysteine residues at position 7 of the A chain and position 7 of the B chain are linked in a disulfide bond, and the cysteine residues at position 20 of the A chain and 19 of the B chain are linked in a disulfide bond.

The term “insulin analogue” as used herein includes any heterodimer analogue or single-chain analogue that comprises one or more modification(s) of the native A-chain peptide and/or B-chain peptide. Modifications include but are not limited to substituting an amino acid for the native amino acid at a position selected from A4, A5, A8, A9, A10, A12, A13, A14, A15, A16, A17, A18, A19, A21, B1, B2, B3, B4, B5, B9, B10, B13, B14, B15, B16, B17, B18, B20, B21, B22, B23, B26, B27, B28, B29, and B30; deleting any or all of positions B1-4 and B26-30; or conjugating directly or by a polymeric or non-polymeric linker one or more acyl, polyethylglycine (PEG), or saccharide moiety (moieties); or any combination thereof. As exemplified by the N-linked glycosylated insulin analogues disclosed herein, the term further includes any insulin heterodimer and single-chain analogue that has been modified to have at least one N-linked glycosylation site and in particular, embodiments in which the N-linked glycosylation site is linked to or occupied by an N-glycan. Examples of insulin analogues include but are not limited to the heterodimer and single-chain analogues disclosed in published international application WO20100080606, WO2009/099763, and WO2010080609, the disclosures of which are incorporated herein by reference. Examples of single-chain insulin analogues also include but are not limited to those disclosed in published International Applications WO9634882, WO95516708, WO2005054291, WO2006097521, WO2007104734, WO2007104736, WO2007104737, WO2007104738, WO2007096332, WO2009132129; U.S. Pat. Nos. 5,304,473 and 6,630,348; and Kristensen et al., Biochem. J. 305: 981-986 (1995), the disclosures of which are each incorporated herein by reference.

The term “insulin analogues” further includes single-chain and heterodimer polypeptide molecules that have little or no detectable activity at the insulin receptor but which have been modified to include one or more amino acid modifications or substitutions to have an activity at the insulin receptor that has at least 1%, 10%, 50%, 75%, or 90% of the activity at the insulin receptor as compared to native insulin and which further includes at least one N-linked glycosylation site. In particular aspects, the insulin analogue is a partial agonist that has from 2× to 100× less activity at the insulin receptor as does native insulin. In other aspects, the insulin analogue has enhanced activity at the insulin receptor, for example, the IGF^{B16B17} derivative peptides disclosed in published international application WO2010080607 (which is incorporated herein by reference). These insulin analogues, which have reduced activity at the insulin growth hormone receptor and enhanced activity at the insulin receptor, include both heterodimers and single-chain analogues.

As used herein, the term “single-chain insulin” or “single-chain insulin analogue” encompasses a group of structurally-related proteins wherein the A-chain peptide or functional analogue and the B-chain peptide or functional analogue are covalently linked by a peptide or polypeptide of 2 to 35 amino acids or non-peptide polymeric or non-polymeric linker and which has at least 1%, 10%, 50%, 75%, or 90% of the activity of insulin at the insulin receptor as compared to native insulin. The single-chain insulin or insulin analogue further includes three disulfide bonds: the first disulfide bond is between the cysteine residues at positions 6 and 11 of the A-chain or functional analogue thereof, the second disulfide bond is between the cysteine residues at position 7 of the A-chain or functional analogue thereof and position 7 of the B-chain or functional analogue thereof, and the third disulfide bond is between the cysteine residues at position 20 of the A-chain or functional analogue thereof and position 19 of the B-chain or functional analogue thereof.

As used herein, the term “connecting peptide” or “C-peptide” refers to the connection moiety “C” of the B-C-A polypeptide sequence of a single chain preproinsulin-like molecule. Specifically, in the natural insulin chain, the C-peptide connects the amino acid at position 30 of the B-chain and the amino acid at position 1 of the A-chain. The term can refer to both the native insulin C-peptide (SEQ ID NO:30), the monkey C-peptide, and any other peptide from 3 to 35 amino acids that connects the B-chain to the A-chain thus is meant to encompass any peptide linking the B-chain peptide to the A-chain peptide in a single-chain insulin analogue (See for example, U.S. Published application Nos. 20090170750 and 20080057004 and WO9634882) and in insulin precursor molecules such as disclosed in WO9516708 and U.S. Pat. No. 7,105,314.

As used herein, the term “pre-proinsulin analogue precursor” refers to a fusion protein comprising a leader peptide, which targets the prepro-insulin analogue precursor to the secretory pathway of the host cell, fused to the N-terminus of a B-chain peptide or B-chain peptide analogue, which is fused to the N-terminus of a C-peptide which in turn is fused at its C-terminus to the N-terminus of an A-chain peptide or A-chain peptide analogue. The fusion protein may optionally include one or more extension or spacer peptides between the C-terminus of the leader peptide and the N-terminus of the B-chain peptide or B-chain peptide analogue. The extension or spacer peptide when present may protect the N-terminus of the B-chain or B-chain analogue from protease digestion during fermentation. The native human pre-proinsulin has the amino acid sequence shown in SEQ ID NO:35.

As used herein, the term “proinsulin analogue precursor” refers to a molecule in which the signal or pre-peptide of the pre-proinsulin analogue precursor has been removed.

As used herein, the term “insulin analogue precursor” refers to a molecule in which the propeptide of the proinsulin analogue precursor has been removed. The insulin analogue precursor may optionally include the extension or spacer peptide at the N-terminus of the B-chain peptide or B-chain peptide analogue. The insulin analogue precursor is a single-chain molecule since it includes a C-peptide; however, the insulin analogue precursor will contain correctly positioned disulphide bridges (three) as in human insulin and may by one or more subsequent chemical and/or enzymatic processes be converted into a heterodimer or single-chain insulin analogue.

As used herein, the term “leader peptide” refers to a polypeptide comprising a pre-peptide (the signal peptide) and a propeptide.

As used herein, the term “signal peptide” refers to a pre-peptide which is present as an N-terminal peptide on a precursor form of a protein. The function of the signal peptide is to facilitate translocation of the expressed polypeptide to which it is attached into the endoplasmic reticulum. The signal peptide is normally cleaved off in the course of this process. The signal peptide may be heterologous or homologous to the organism used to produce the polypeptide. A number of signal peptides which may be used include the yeast aspartic protease 3 (YAP3) signal peptide or any functional analogue (Egel-Mitani et al. YEAST 6:127 137 (1990) and U.S. Pat. No. 5,726,038) and the signal peptide of the Saccharomyces cerevisiae mating factor α1 gene (ScMF α 1) gene (Thorner (1981) in The Molecular Biology of the Yeast Saccharomyces cerevisiae, Strathern et al., eds., pp 143 180, Cold Spring Harbor Laboratory, NY and U.S. Pat. No. 4,870,008.

As used herein, the term “propeptide” refers to a peptide whose function is to allow the expressed polypeptide to which it is attached to be directed from the endoplasmic reticulum to the Golgi apparatus and further to a secretory vesicle for secretion into the culture medium (i.e., exportation of the polypeptide across the cell wall or at least through the cellular membrane into the periplasmic space of the yeast cell). The propeptide may be the ScMF α1 (See U.S. Pat. Nos. 4,546,082 and 4,870,008). Alternatively, the pro-peptide may be a synthetic propeptide, which is to say a propeptide not found in nature, including but not limited to those disclosed in U.S. Pat. Nos. 5,395,922; 5,795,746; and 5,162,498 and in WO 9832867. The propeptide will preferably contain an endopeptidase processing site at the C-terminal end, such as a Lys-Arg sequence or any functional analogue thereof.

As used herein with the term “insulin”, the term “desB30” or “B(1-29)” is meant to refer to an insulin B-chain peptide lacking the B30 amino acid residue and “A(1-21)” means the insulin A chain.

As used herein, the term “immediately N-terminal to” is meant to illustrate the situation where an amino acid residue or a peptide sequence is directly linked at its C-terminal end to the N-terminal end of another amino acid residue or amino acid sequence by means of a peptide bond.

As used herein an amino acid “modification” refers to a substitution of an amino acid, or the derivation of an amino acid by the addition and/or removal of chemical groups to/from the amino acid, and includes substitution with any of the 20 amino acids commonly found in human proteins, as well as atypical or non-naturally occurring amino acids. Commercial sources of atypical amino acids include Sigma-Aldrich (Milwaukee, Wis.), ChemPep Inc. (Miami, Fla.), and Genzyme Pharmaceuticals (Cambridge, Mass.). Atypical amino acids may be purchased from commercial suppliers, synthesized de novo, or chemically modified or derivatized from naturally occurring amino acids.

As used herein an amino acid “substitution” refers to the replacement of one amino acid residue by a different amino acid residue. Throughout the application, all references to a particular amino acid position by letter and number (e.g. position A5) refer to the amino acid at that position of either the A-chain (e.g. position A5) or the B-chain (e.g. position B5) in the respective native human insulin A-chain (SEQ ID NO: 33) or B-chain (SEQ ID NO: 25), or the corresponding amino acid position in any analogues thereof.

The term “glycoprotein” is meant to include any glycosylated insulin analogue, including single-chain insulin analogue, comprising one or more attachment groups to which one or more oligosaccharides is covalently linked thereto.

As used herein, an “N-linked glycosylation site” refers to the tri-peptide amino acid sequence NX(S/T) or AsnXaa(Ser/Thr) wherein “N” represents an asparagine (Asn) residue, “X” represents any amino acid (Xaa) except proline (Pro), “S” represents a serine (Ser) residue, and “T” represents a threonine (Thr) residue.

As used herein, the term “N-glycan” and “glycoform” are used interchangeably and refer to the oligosaccharide group per se that is attached by an asparagine-N-acetylglucosamine linkage to an attachment group comprising an N-linked glycosylation site. The N-glycan oligosaccharide group may be attached in vitro to any amino acid residue other than asparagine or in vivo to an asparagine residue comprising an N-linked glycosylation site.

The term “N-linked glycan” refers to an N-glycan in which the N-acetylglucosamine residue at the reducing end is linked in a β1 linkage to the amide nitrogen of an asparagine residue of an attachment group in the protein.

As used herein, the terms “N-linked glycosylated” and “N-glycosylated” are used interchangeably and refer to an N-glycan attached to an attachment group comprising an asparagine residue or an N-linked glycosylation site or motif.

As used herein, the term “N-glycan conjugate” refers to an N-glycan that is conjugated to an attachment group in vitro. The attachment group may or may not include an asparagine residue.

As used herein, the term “glycosylated insulin or insulin analogue” refers to an insulin or insulin analogue to which an N-glycan is attached thereto either in vivo or in vitro.

As used herein, the term “in vivo glycosylation” or “in vivo N-glycosylation” or “in vivo N-linked glycosylation” refers to the attachment of an oligosaccharide or glycan moiety to an asparagine residue of an N-linked glycosylation site occurring in vivo, i.e., during posttranslational processing in a glycosylating cell expressing the polypeptide by way of N-linked glycosylation. The exact oligosaccharide structure depends, to a large extent, on the host cell used to produce the glycosylated protein or polypeptide.

As used herein, the term “in vitro glycosylation” refers to a synthetic glycosylation performed in vitro, normally involving covalently linking an N-glycan having a functional group capable of being conjugated or linked to an attachment group of a polypeptide, optionally using a cross-linking agent to provide an N-glycan conjugate. In vitro glycosylation further includes chemically synthesizing the protein or polypeptide wherein an amino acid covalently linked to an N-glycan is incorporated into the protein or polypeptide during synthesis. In vivo and in vitro glycosylation are discussed in detail further below.

The term “attachment group” is intended to indicate a functional group of the polypeptide, in particular of an amino acid residue thereof, capable of being covalently linked to a macromolecular substance such as an oligosaccharide or glycan, a polymer molecule, a lipophilic molecule, or an organic derivatizing agent.

For in vivo N-glycosylation, the term “attachment group” is used in an unconventional way to indicate the amino acid residues constituting an “N-linked glycosylation site” or “N-glycosylation site” comprising N-X-S/T, wherein X is any amino acid except proline. Although the asparagine (N) residue of the N-glycosylation site is where the oligosaccharide or glycan moiety is attached during glycosylation, such attachment cannot be achieved unless the other amino acid residues of the N-glycosylation site are present. While the N-linked glycosylated insulin analogue precursor will include all three amino acids comprising the “attachment group” to enable in vivo N-glycosylation, the N-linked glycosylated insulin analogue may be processed subsequently to lack X and/or S/T. Accordingly, when the conjugation is to be achieved by N-glycosylation, the term “amino acid residue comprising an attachment group for the oligosaccharide or glycan” as used in connection with alterations of the amino acid sequence of the polypeptide is to be understood as meaning that one or more amino acid residues constituting an N-glycosylation site are to be altered in such a manner that a functional N-glycosylation site is introduced into the amino acid sequence. The attachment group may be present in the insulin analogue precursor but in the heterodimer insulin analogue one or two of the amino acid residues comprising the attachment site but not the asparagine (N) residue linked to the oligosaccharide or glycan may be removed. For example, an insulin analogue precursor may comprise an attachment group consisting of NKT at positions B28, 29, and 30, respectively, but the mature heterodimer of the analogue may be a desB30 insulin analogue wherein the T at position 30 has been removed.

In general, for the conjugate disclosed herein comprising an introduced amino acid residue with an attachment group for the macromolecular substance, it is preferred that the macromolecular substance is attached to the introduced amino acid residue. More specifically, it is generally understood for the positions specifically indicated herein as attachment sites for the macromolecular substance, that the conjugate of the invention comprises at least the macromolecular substance attached to one of said positions.

As used herein, “N-glycans” have a common pentasaccharide core of Man₃GlcNAc₂ (“Man” refers to mannose; “Glc” refers to glucose; and “NAc” refers to N-acetyl; GlcNAc refers to N-acetylglucosamine). Usually, N-glycan structures are presented with the non-reducing end to the left and the reducing end to the right. The reducing end of the N-glycan is the end that is attached to the Asn residue comprising the glycosylation site on the protein. N-glycans differ with respect to the number of branches (antennae) comprising peripheral sugars (e.g., GlcNAc, galactose, fucose and sialic acid) that are added to the Man₃GlcNAc₂ (“Man₃”) core structure which is also referred to as the “trimannose core”, the “pentasaccharide core” or the “paucimannose core”. N-glycans are classified according to their branched constituents (e.g., high mannose, complex or hybrid). A “high mannose” type N-glycan has five or more mannose residues. A “complex” type N-glycan typically has at least one GlcNAc attached to the 1,3 mannose arm and at least one GlcNAc attached to the 1,6 mannose arm of a “trimannose” core. Complex N-glycans may also have galactose (“Gal”) or N-acetylgalactosamine (“GalNAc”) residues that are optionally modified with sialic acid or derivatives (e.g., “NANA” or “NeuAc”, where “Neu” refers to neuraminic acid and “Ac” refers to acetyl). Complex N-glycans may also have intrachain substitutions comprising “bisecting” GlcNAc and core fucose (“Fuc”). Complex N-glycans may also have multiple antennae on the “trimannose core,” often referred to as “multiple antennary glycans.” A “hybrid” N-glycan has at least one GlcNAc on the terminal of the 1,3 mannose arm of the trimannose core and zero or more mannoses on the 1,6 mannose arm of the trimannose core. N-glycans consisting of a Man₃GlcNAc₂ structure are called paucimannose. The various N-glycans are also referred to as “glycoforms.”

With respect to complex N-glycans, the terms “G-2”, “G-1”, “G0”, “G1”, “G2”, “A1”, and “A2” mean the following. “G-2” refers to an N-glycan structure that can be characterized as Man₃GlcNAc₂; the term “G-1” refers to an N-glycan structure that can be characterized as GlcNAcMan₃GlcNAc₂; the term “G0” refers to an N-glycan structure that can be characterized as GlcNAc₂Man₃GlcNAc₂; the term “G1” refers to an N-glycan structure that can be characterized as GalGlcNAc₂Man₃GlcNAc₂; the term “G2” refers to an N-glycan structure that can be characterized as Gal₂GlcNAc₂Man₃GlcNAc₂; the term “A1” refers to an N-glycan structure that can be characterized as NANAGal₂GlcNAc₂Man₃GlcNAc₂; and, the term “A2” refers to an N-glycan structure that can be characterized as NANA₂Gal₂GlcNAc₂Man₃GlcNAc₂. Unless otherwise indicated, the terms G-2”, “G-1”, “G0”, “G1”, “G2”, “A1”, and “A2” refer to N-glycan species that lack fucose attached to the GlcNAc residue at the reducing end of the N-glycan. When the term includes an “F”, the “F” indicates that the N-glycan species contains a fucose residue on the GlcNAc residue at the reducing end of the N-glycan. For example, G0F, G1F, G2F, A1F, and A2F all indicate that the N-glycan further includes a fucose residue attached to the GlcNAc residue at the reducing end of the N-glycan. Lower eukaryotes such as yeast and filamentous fungi do not normally produce N-glycans that produce fucose.

With respect to multiantennary N-glycans, the term “multiantennary N-glycan” refers to N-glycans that further comprise a GlcNAc residue on the mannose residue comprising the non-reducing end of the 1,6 arm or the 1,3 arm of the N-glycan or a GlcNAc residue on each of the mannose residues comprising the non-reducing end of the 1,6 arm and the 1,3 arm of the N-glycan. Thus, multiantennary N-glycans can be characterized by the formulas GlcNAc₍₂₋₄₎Man₃GlcNAc₂, Gal₍₁₋₄₎GlcNAc₍₂₋₄₎Man₃GlcNAc₂, or NANA₍₁₋₄₎Gal₍₁₋₄₎GlcNAc₍₂₋₄₎Man₃GlcNAc₂. The term “1-4” refers to 1, 2, 3, or 4 residues.

With respect to bisected N-glycans, the term “bisected N-glycan” refers to N-glycans in which a GlcNAc residue is linked to the mannose residue at the non-reducing end of the N-glycan. A bisected N-glycan can be characterized by the formula GlcNAc₃Man₃GlcNAc₂ wherein each mannose residue is linked at its non-reducing end to a GlcNAc residue. In contrast, when a multiantennary N-glycan is characterized as GlcNAc₃Man₃GlcNAc₂, the formula indicates that two GlcNAc residues are linked to the mannose residue at the non-reducing end of one of the two arms of the N-glycans and one GlcNAc residue is linked to the mannose residue at the non-reducing end of the other arm of the N-glycan.

Abbreviations used herein are of common usage in the art, see, e.g., abbreviations of sugars, above. Other common abbreviations include “PNGase”, or “glycanase” which all refer to glycopeptide N-glycosidase; glycopeptidase; N-oligosaccharide glycopeptidase; N-glycanase; glycopeptidase; Jack-bean glycopeptidase; PNGase A; PNGase F; glycopeptide N-glycosidase (EC 3.5.1.52, formerly EC 3.2.2.18).

The term “recombinant host cell” (“expression host cell”, “expression host system”, “expression system” or simply “host cell”), as used herein, is intended to refer to a cell into which a recombinant vector has been introduced. It should be understood that such terms are intended to refer not only to the particular subject cell but to the progeny of such a cell. Because certain modifications may occur in succeeding generations due to either mutation or environmental influences, such progeny may not, in fact, be identical to the parent cell, but are still included within the scope of the term “host cell” as used herein. A recombinant host cell may be an isolated cell or cell line grown in culture or may be a cell which resides in a living tissue or organism. Host cells may be yeast, fungi, mammalian cells, plant cells, insect cells, and prokaryotes and archaea that have been genetically engineered to produce glycoproteins.

When referring to “mole percent” or “mole %” of a glycan present in a preparation of a glycoprotein, the term means the molar percent of a particular glycan present in the pool of N-linked oligosaccharides released when the protein preparation is treated with PNGase and then quantified by a method that is not affected by glycoform composition, (for instance, labeling a PNGase released glycan pool with a fluorescent tag such as 2-aminobenzamide and then separating by high performance liquid chromatography or capillary electrophoresis and then quantifying glycans by fluorescence intensity). For example, 50 mole percent GlcNAc₂Man₃GlcNAc₂Gal₂NANA₂ means that 50 percent of the released glycans are GlcNAc₂Man₃GlcNAc₂Gal₂NANA₂ and the remaining 50 percent are comprised of other N-linked oligosaccharides. In embodiments, the mole percent of a particular glycan in a preparation of glycoprotein will be between 20% and 100%, preferably above 25%, 30%, 35%, 40% or 45%, more preferably above 50%, 55%, 60%, 65% or 70% and most preferably above 75%, 80% 85%, 90% or 95%.

The term “operably linked” expression control sequences refers to a linkage in which the expression control sequence is contiguous with the gene of interest to control the gene of interest, as well as expression control sequences that act in trans or at a distance to control the gene of interest.

The term “expression control sequence” or “regulatory sequences” are used interchangeably and as used herein refer to polynucleotide sequences which are necessary to affect the expression of coding sequences to which they are operably linked. Expression control sequences are sequences which control the transcription, post-transcriptional events and translation of nucleic acid sequences. Expression control sequences include appropriate transcription initiation, termination, promoter and enhancer sequences; efficient RNA processing signals such as splicing and polyadenylation signals; sequences that stabilize cytoplasmic mRNA; sequences that enhance translation efficiency (e.g., ribosome binding sites); sequences that enhance protein stability; and when desired, sequences that enhance protein secretion. The nature of such control sequences differs depending upon the host organism; in prokaryotes, such control sequences generally include promoter, ribosomal binding site, and transcription termination sequence. The term “control sequences” is intended to include, at a minimum, all components whose presence is essential for expression, and can also include additional components whose presence is advantageous, for example, leader sequences and fusion partner sequences.

The term “transfect”, “transfection”, “transfecting” and the like refer to the introduction of a heterologous nucleic acid into eukaryote cells, both higher and lower eukaryote cells. Historically, the term “transformation” has been used to describe the introduction of a nucleic acid into a prokaryote, yeast, or fungal cell; however, the term “transfection” is also used to refer to the introduction of a nucleic acid into any prokaryotic or eukaryote cell, including yeast and fungal cells. Furthermore, introduction of a heterologous nucleic acid into prokaryotic or eukaryotic cells may also occur by viral or bacterial infection or ballistic DNA transfer, and the term “transfection” is also used to refer to these methods in appropriate host cells.

The term “eukaryotic” refers to a nucleated cell or organism, and includes insect cells, plant cells, mammalian cells, animal cells and lower eukaryotic cells.

The term “lower eukaryotic cells” includes yeast and filamentous fungi. Yeast and filamentous fungi include, but are not limited to Pichia pastoris, Pichia finlandica, Pichia trehalophila, Pichia koclamae, Pichia membranaefaciens, Pichia minuta (Ogataea minuta, Pichia lindneri), Pichia opuntiae, Pichia thermotolerans, Pichia salictaria, Pichia guercuum, Pichia pijperi, Pichia stiptis, Pichia methanolica, Pichia sp., Saccharomyces cerevisiae, Saccharomyces sp., Hansenula polymorpha, Kluyveromyces sp., Kluyveromyces lactis, Yarrowia lipolytica, Candida albicans, any Aspergillus sp., Aspergillus nidulans, Aspergillus niger, Aspergillus oryzae, Trichoderma reesei, Chrysosporium lucknowense, Fusarium sp., Fusarium gramineum, Fusarium venenatum, Physcomitrella patens and Neurospora crassa.

As used herein, the term “consisting essentially of” will be understood to imply the inclusion of a stated integer or group of integers; while excluding modifications or other integers which would materially affect or alter the stated integer. For example, with respect to a species of N-glycans attached to an insulin or insulin analogue, the term “consisting essentially of” a stated N-glycan will be understood to include the N-glycan whether or not that N-glycan is fucosylated at the N-acetylglucosamine (GlcNAc) which is directly linked to the asparagine residue of the glycoprotein provided that for the particular N-glycan species the fucose does not materially affect the glycosylated insulin or insulin analogue compared to the glycosylated insulin or insulin analogue in which the N-glycan lacks the fucose.

As used herein, the term “predominantly” or variations such as “the predominant” or “which is predominant” will be understood to mean the glycan species that has the highest mole percent (%) of total neutral N-glycans after the insulin analogue has been treated with PNGase and released glycans analyzed by mass spectroscopy, for example, MALDI-TOF MS or HPLC. In other words, the phrase “predominantly” is defined as an individual entity, such as a specific glycoform, is present in greater mole percent than any other individual entity. For example, if a composition consists of species A at 40 mole percent, species B at 35 mole percent and species C at 25 mole percent, the composition comprises predominantly species A, and species B would be the next most predominant species. Some host cells may produce compositions comprising neutral N-glycans and charged N-glycans such as mannosylphosphate. Therefore, a composition of glycoproteins can include a plurality of charged and uncharged or neutral N-glycans. In the present invention, it is within the context of the total plurality of neutral N-glycans in the composition in which the predominant N-glycan determined. Thus, as used herein, “predominant N-glycan” means that of the total plurality of neutral N-glycans in the composition, the predominant N-glycan is of a particular structure.

As used herein, the term “essentially free of” a particular sugar residue, such as fucose, or galactose and the like, is used to indicate that the glycoprotein composition is substantially devoid of N-glycans which contain such residues. Expressed in terms of purity, essentially free means that the amount of N-glycan structures containing such sugar residues does not exceed 10%, and preferably is below 5%, more preferably below 1%, most preferably below 0.5%, wherein the percentages are by weight or by mole percent. Thus, substantially all of the N-glycan structures in an insulin analogue composition disclosed herein are free of, for example, fucose, or galactose, or both.

As used herein, an insulin analogue composition “lacks” or “is lacking” a particular sugar residue, such as fucose or galactose, when no detectable amount of such sugar residue is present on the N-glycan structures at any time. For example, in preferred embodiments of the present invention, the insulin analogue compositions are produced by lower eukaryotic organisms, as defined above, including yeast (for example, Pichia sp.; Saccharomyces sp.; Kluyveromyces sp.; Aspergillus sp.), and will “lack fucose,” because the cells of these organisms do not have the enzymes needed to produce fucosylated N-glycan structures. Thus, the term “essentially free of fucose” encompasses the term “lacking fucose.” However, a composition may be “essentially free of fucose” even if the composition at one time contained fucosylated N-glycan structures or contains limited, but detectable amounts of fucosylated N-glycan structures as described above.

As used herein, the term “pharmaceutically acceptable carrier” includes any of the standard pharmaceutical carriers, such as a phosphate buffered saline solution, water, emulsions such as an oil/water or water/oil emulsion, and various types of wetting agents. The term also encompasses any of the agents approved by a regulatory agency of the U.S. Federal government or listed in the U.S. Pharmacopeia for use in animals, including humans.

As used herein the term “pharmaceutically acceptable salt” refers to salts of compounds that retain the biological activity of the parent compound, and which are not biologically or otherwise undesirable. Many of the compounds disclosed herein are capable of forming acid and/or base salts by virtue of the presence of amino and/or carboxyl groups or groups similar thereto.

Pharmaceutically acceptable base addition salts can be prepared from inorganic and organic bases. Salts derived from inorganic bases, include by way of example only, sodium, potassium, lithium, ammonium, calcium and magnesium salts. Salts derived from organic bases include, but are not limited to, salts of primary, secondary and tertiary amines.

Pharmaceutically acceptable acid addition salts may be prepared from inorganic and organic acids. Salts derived from inorganic acids include hydrochloric acid, hydrobromic acid, sulfuric acid, nitric acid, phosphoric acid, and the like. Salts derived from organic acids include acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluene-sulfonic acid, salicylic acid, and the like.

As used herein, the term “treating” includes prophylaxis of the specific disorder or condition, or alleviation of the symptoms associated with a specific disorder or condition and/or preventing or eliminating said symptoms. For example, as used herein the term “treating diabetes” will refer in general to maintaining glucose blood levels near normal levels and may include increasing or decreasing blood glucose levels depending on a given situation.

As used herein an “effective” amount or a “therapeutically effective amount” of an insulin analogue refers to a nontoxic but sufficient amount of an insulin analogue to provide the desired effect. For example one desired effect would be the prevention or treatment of hyperglycemia. The amount that is “effective” will vary from subject to subject, depending on the age and general condition of the individual, mode of administration, and the like. Thus, it is not always possible to specify an exact “effective amount.” However, an appropriate “effective” amount in any individual case may be determined by one of ordinary skill in the art using routine experimentation.

The term, “parenteral” means not through the alimentary canal but by some other route such as intranasal, inhalation, subcutaneous, intramuscular, intraspinal, or intravenous.

As used herein, the term “pharmacokinetic” refers to in vivo properties of an insulin or insulin analogue commonly used in the field that relate to the liberation, absorption, distribution, metabolism, and elimination of the protein. Such pharmacokinetic properties include, but are not limited to, dose, dosing interval, concentration, elimination rate, elimination rate constant, area under curve, volume of distribution, clearance in any tissue or cell, proteolytic degradation in blood, bioavailability, binding to plasma, half-life, first-pass elimination, extraction ratio, Cₘₐₓ, tₘₐₓ, Cₘᵢₙ, rate of absorption, and fluctuation.

As used herein, the term “pharmacodynamic” refers to in vivo properties of an insulin or insulin analogue commonly used in the field that relate to the physiological effects of the protein. Such pharmacokinetic properties include, but are not limited to, maximal glucose infusion rate, time to maximal glucose infusion rate, and area under the glucose infusion rate curve.

### BRIEF DESCRIPTION OF THE DRAWINGS

FIG. 1 shows examples of where mutations may be made to the native insulin amino acid sequence that would generate N-linked glycosylation sites in the native insulin amino acid sequence that could be glycosylated in vivo to generate N-glycosylated insulin analogues. The shown mutations may be alone or in combination. The amino acid sequences shown for the A- and B-chain peptides (SEQ ID NOs:33 and 25, respectively) are those of wild-type human insulin. Similar mutations to generate N-glycosylation sites may also be constructed from any other insulin analogue, including lispro, aspart, glulisine, glargine, and determir.

FIG. 2 shows examples of N-glycan structures that can be attached to the asparagine residue in the motif Asn-Xaa-Ser/Thr wherein Xaa is any amino acid other than proline or attached to any amino acid in vitro.

FIG. 3A and FIG. 3B show[s] the pharmacokinetics of two glycosylated insulin analogues. FIG. 3A shows the circulating insulin analogue levels during an insulin tolerance test (ITT) for P28N des(B30) GS5.0 (galactose-terminated N-glycans) insulin analogue and P28N des(B30) GS6.0 (sialic acid-terminated N-glycans) insulin analogue compared to that of NOVOLIN R and NOVOLIN des(B30). In FIG. 3B, shows the AUC for, from left to right, vehicle, Novolin, P28N des(B30) GS5.0, P28N des(B30) GS 6.0, and Novolin R.

FIG. 4 shows the in vivo activities of two N-glycosylated insulin analogues. Shown are the glucose levels during a mouse ITT for P28N des(B30) GS5.0 (galactose-terminated N-glycans) insulin analogue and P28N des(B30) GS6.0 (sialic acid-terminated N-glycans) insulin analogue compared to that of NOVOLIN R and NOVOLIN des(B30).

FIG. 5A, FIG. 5B, FIG. 5C, and FIG. 5D show[s] in vitro activities of the two N-glycosylated insulin analogues at the insulin and insulin-like growth factor (IGF-1) receptors. Shown are the insulin receptor binding (FIG. 5A), insulin receptor phosphorylation (FIG. 5C), and IGF-1 receptor binding (FIG. 5B) for P28N des(B30) GS5.0 (galactose-terminated N-glycans) insulin analogue and P28N des(B30) GS6.0 (sialic acid-terminated N-glycans) insulin analogue compared to that of NOVOLIN R and NOVOLIN des(B30). FIG. 5D shows the EC50 values for the insulin receptor binding, insulin receptor phosphorylation, and IGF-1 receptor binding.

FIG. 6 shows map of plasmid pGLY4362, which is a roll-in integration plasmid that targets the TRP2 or AOX1p locus, includes an expression cassette encoding an insulin precursor fusion protein comprising a Yps1ss peptide fused to a TA57 propeptide fused to an N-terminal spacer fused to the human insulin B-chain with a P28N substitution fused to a C-peptide consisting of the amino acid sequence AAK fused to the human insulin A-chain.

FIG. 7 shows map of plasmid pGLY7679, which is a roll-in integration plasmid that targets the TRP2 or AOX1p locus, includes an expression cassette encoding an insulin precursor fusion protein comprising a Yps1ss peptide fused to a TA57 propeptide fused to an N-terminal spacer peptide fused to the human insulin B-chain with a P28N substitution fused to a C-peptide consisting of the amino acid sequence A(10×HIS)AK fused to the human insulin A-chain.

FIG. 8 shows map of plasmid pGLY7680, which is a roll-in integration plasmid that targets the TRP2 or AOX1p locus, includes an expression cassette encoding an insulin precursor fusion protein comprising a S. cerevisiae alpha mating factor signal sequence and propeptide fused to the human insulin B-chain with a P28N substitution fused to a C-peptide consisting of the amino acid sequence RR fused to the human insulin A-chain.

FIG. 9 shows map of plasmid pGLY9290, which is a roll-in integration plasmid that targets the TRP2 or AOX1p locus, includes an expression cassette encoding an insulin precursor fusion protein comprising a S. cerevisiae alpha mating factor signal sequence and propeptide fused to the human insulin B-chain with a P28N substitution fused to a C-peptide consisting of the amino acid sequence RR fused to the human insulin A-chain with an N21G substitution.

FIG. 10 shows map of plasmid pGLY9295, which is a roll-in integration plasmid that targets the TRP2 or AOX1p locus, includes an expression cassette encoding an insulin precursor fusion protein comprising a S. cerevisiae alpha mating factor signal sequence and propeptide fused to an N-terminal HIS spacer peptide fused to the human insulin B-chain with a P28N substitution fused to a C-peptide consisting of the amino acid sequence RR fused to the human insulin A-chain with an N21G substitution.

FIG. 11 shows map of plasmid pGLY9310, which is a roll-in integration plasmid that targets the TRP2 or AOX1p locus, includes an expression cassette encoding an insulin precursor fusion protein comprising a S. cerevisiae alpha mating factor signal sequence and propeptide fused to the human insulin B-chain with a P28N substitution fused to a C-peptide consisting of the amino acid sequence RR fused to the human insulin A-chain with an N21G substitution.

FIG. 12 shows map of plasmid pGLY9311, which is a roll-in integration plasmid that targets the TRP2 or AOX1p locus, includes an expression cassette encoding an insulin precursor fusion protein comprising a S. cerevisiae alpha mating factor signal sequence and propeptide fused to an N-terminal MYC spacer peptide fused to the human insulin B-chain with a P28N substitution fused to a C-peptide consisting of the amino acid sequence TA(10×HIS)AK (SEQ ID NO:32) fused to the human insulin A-chain.

FIGS. 13A, 13B, 13C, and 13D show the construction of strains YGLY12897 and YGLY12900. Both strains are capable of producing glycoproteins, including the insulin analogues disclosed herein, comprising sialic-acid terminated N-glycans.

FIG. 14 shows a map of plasmid pGLY6. Plasmid pGLY6 is an integration vector that targets the URA5 locus and contains a nucleic acid molecule comprising the S. cerevisiae invertase gene or transcription unit (ScSUC2) flanked on one side by a nucleic acid molecule comprising a nucleotide sequence from the 5′ region of the P. pastoris URA5 gene (PpURA5-5′) and on the other side by a nucleic acid molecule comprising the a nucleotide sequence from the 3′ region of the P. pastoris URA5 gene (PpURA5-3′).

FIG. 15 shows a map of plasmid pGLY40. Plasmid pGLY40 is an integration vector that targets the OCH1 locus and contains a nucleic acid molecule comprising the P. pastoris URA5 gene or transcription unit (PpURA5) flanked by nucleic acid molecules comprising lacZ repeats (lacZ repeat) which in turn is flanked on one side by a nucleic acid molecule comprising a nucleotide sequence from the 5′ region of the OCH1 gene (PpOCH1-5′) and on the other side by a nucleic acid molecule comprising a nucleotide sequence from the 3′ region of the OCH1 gene (PpOCH1-3′).

FIG. 16 shows a map of plasmid pGLY43a. Plasmid pGLY43a is an integration vector that targets the BMT2 locus and contains a nucleic acid molecule comprising the K. lactis UDP-N-acetylglucosamine (UDP-GlcNAc) transporter gene or transcription unit (KlGlcNAc Transp.) adjacent to a nucleic acid molecule comprising the P. pastoris URA5 gene or transcription unit (PpURA5) flanked by nucleic acid molecules comprising lacZ repeats (lacZ repeat). The adjacent genes are flanked on one side by a nucleic acid molecule comprising a nucleotide sequence from the 5′ region of the BMT2 gene (PpPBS2-5′) and on the other side by a nucleic acid molecule comprising a nucleotide sequence from the 3′ region of the BMT2 gene (PpPBS2-3′).

FIG. 17 shows a map of plasmid pGLY48. Plasmid pGLY48 is an integration vector that targets the MNN4L1 locus and contains an expression cassette comprising a nucleic acid molecule encoding the mouse homologue of the UDP-GlcNAc transporter (MmGlcNAc Transp.) open reading frame (ORF) operably linked at the 5′ end to a nucleic acid molecule comprising the P. pastoris GAPDH promoter (PpGAPDH Prom) and at the 3′ end to a nucleic acid molecule comprising the S. cerevisiae CYC termination sequence (ScCYC TT) adjacent to a nucleic acid molecule comprising the P. pastoris URA5 gene or transcription unit (PpURA5) flanked by lacZ repeats (lacZ repeat) and in which the expression cassettes together are flanked on one side by a nucleic acid molecule comprising a nucleotide sequence from the 5′ region of the P. pastoris MNN4L1 gene (PpMNN4L1-5′) and on the other side by a nucleic acid molecule comprising a nucleotide sequence from the 3′ region of the MNN4L1 gene (PpMNN4L1-3′).

FIG. 18 shows as map of plasmid pGLY45. Plasmid pGLY45 is an integration vector that targets the PNO1/MNN4 loci contains a nucleic acid molecule comprising the P. pastoris URA5 gene or transcription unit (PpURA5) flanked by nucleic acid molecules comprising lacZ repeats (lacZ repeat) which in turn is flanked on one side by a nucleic acid molecule comprising a nucleotide sequence from the 5′ region of the PNO1 gene (PpPNO1-5′) and on the other side by a nucleic acid molecule comprising a nucleotide sequence from the 3′ region of the MNN4 gene (PpMNN4-3′).

FIG. 19 shows a map of plasmid pGLY1430. Plasmid pGLY1430 is a KINKO integration vector that targets the ADE1 locus without disrupting expression of the locus and contains in tandem four expression cassettes encoding (1) the human GlcNAc transferase I catalytic domain (codon optimized) fused at the N-terminus to P. pastoris SEC12 leader peptide (CO-NA10), (2) mouse homologue of the UDP-GlcNAc transporter (MmTr), (3) the mouse mannosidase IA catalytic domain (FB) fused at the N-terminus to S. cerevisiae SEC12 leader peptide (FB8), and (4) the P. pastoris URA5 gene or transcription unit (PpURA5) flanked by lacZ repeats (lacZ). All flanked by the 5′ region of the ADE1 gene and ORF (ADE1 5′ and ORF) and the 3′ region of the ADE1 gene (PpADE1-3′). PpPMA1 prom is the P. pastoris PMA1 promoter; PpPMA1 TT is the P. pastoris PMA1 termination sequence; SEC4 is the P. pastoris SEC4 promoter; OCH1 TT is the P. pastoris OCH1 termination sequence; ScCYC TT is the S. cerevisiae CYC termination sequence; PpOCH1 Prom is the P. pastoris OCH1 promoter; PpALG3 TT is the P. pastoris ALG3 termination sequence; and PpGAPDH is the P. pastoris GADPH promoter.

FIG. 20 shows a map of plasmid pGLY582. Plasmid pGLY582 is an integration vector that targets the HIS1 locus and contains in tandem four expression cassettes encoding (1) the S. cerevisiae UDP-glucose epimerase (ScGAL10), (2) the human galactosyltransferase I (hGalT) catalytic domain fused at the N-terminus to the S. cerevisiae KRE2-s leader peptide (33), (3) the P. pastoris URA5 gene or transcription unit (PpURA5) flanked by lacZ repeats (lacZ repeat), and (4) the D. melanogaster UDP-galactose transporter (DmUGT). All flanked by the 5′ region of the HIS1 gene (PpHIS1-5′) and the 3′ region of the HIS1 gene (PpHIS1-3′). PMA1 is the P. pastoris PMA promoter; PpPMA1 TT is the P. pastoris PMA1 termination sequence; GAPDH is the P. pastoris GADPH promoter and ScCYC TT is the S. cerevisiae CYC termination sequence; PpOCH1 Prom is the P. pastoris OCH1 promoter and PpALG12 TT is the P. pastoris ALG12 termination sequence.

FIG. 21 shows a map of plasmid pGLY167b. Plasmid pGLY167b is an integration vector that targets the ARG1 locus and contains in tandem three expression cassettes encoding (1) the D. melanogaster mannosidase II catalytic domain (codon optimized) fused at the N-terminus to S. cerevisiae MNN2 leader peptide (CO-KD53), (2) the P. pastoris HIS1 gene or transcription unit, and (3) the rat N-acetylglucosamine (GlcNAc) transferase II catalytic domain (codon optimized) fused at the N-terminus to S. cerevisiae MNN2 leader peptide (CO-TC54). All flanked by the 5′ region of the ARG1 gene (PpARG1-5′) and the 3′ region of the ARG1 gene (PpARG1-3′). PpPMA1 prom is the P. pastoris PMA1 promoter; PpPMA1 TT is the P. pastoris PMA1 termination sequence; PpGAPDH is the P. pastoris GADPH promoter; ScCYC TT is the S. cerevisiae CYC termination sequence; PpOCH1 Prom is the P. pastoris OCH1 promoter; and PpALG12 TT is the P. pastoris ALG12 termination sequence.

FIG. 22 shows a map of plasmid pGLY3411 (pSH1092). Plasmid pGLY3411 (pSH1092) is an integration vector that contains the expression cassette comprising the P. pastoris URA5 gene or transcription unit (PpURA5) flanked by lacZ repeats (lacZ repeat) flanked on one side with the 5′ nucleotide sequence of the P. pastoris BMT4 gene (PpPBS4 5′) and on the other side with the 3′ nucleotide sequence of the P. pastoris BMT4 gene (PpPBS4 3′).

FIG. 23 shows a map of plasmid pGLY3419 (pSH1110). Plasmid pGLY3430 (pSH1115) is an integration vector that contains an expression cassette comprising the P. pastoris URA5 gene or transcription unit (PpURA5) flanked by lacZ repeats (lacZ repeat) flanked on one side with the 5′ nucleotide sequence of the P. pastoris BMT1 gene (PBS1 5′) and on the other side with the 3′ nucleotide sequence of the P. pastoris BMT1 gene (PBS1 3′)

FIG. 24 shows a map of plasmid pGLY3421 (pSH1106). Plasmid pGLY4472 (pSH1186) contains an expression cassette comprising the P. pastoris URA5 gene or transcription unit (PpURA5) flanked by lacZ repeats (lacZ repeat) flanked on one side with the 5′ nucleotide sequence of the P. pastoris BMT3 gene (PpPBS3 5′) and on the other side with the 3′ nucleotide sequence of the P. pastoris BMT3 gene (PpPBS3 3′).

FIG. 25 shows a map of plasmid pGLY2456. Plasmid pGLY2456 is a KINKO integration vector that targets the TRP2 locus without disrupting expression of the locus and contains six expression cassettes encoding (1) the mouse CMP-sialic acid transporter codon optimized (CO mCMP-Sia Transp), (2) the human UDP-GlcNAc 2-epimerase/N-acetylmannosamine kinase codon optimized (CO hGNE), (3) the Pichia pastoris ARG1 gene or transcription unit, (4) the human CMP-sialic acid synthase codon optimized (CO hCMP-NANA S), (5) the human N-acetylneuraminate-9-phosphate synthase codon optimized (CO hSIAP S), and, (6) the mouse a-2,6-sialyltransferase catalytic domain codon optimized fused at the N-terminus to S. cerevisiae KRE2 leader peptide (comST6-33). All flanked by the 5′ region of the TRP2 gene and ORF (PpTRP2 5′) and the 3′ region of the TRP2 gene (PpTRP2-3′). PpPMA1 prom is the P. pastoris PMA1 promoter; PpPMA1 TT is the P. pastoris PMA1 termination sequence; CYC TT is the S. cerevisiae CYC termination sequence; PpTEF Prom is the P. pastoris TEF1 promoter; PpTEF TT is the P. pastoris TEF1 termination sequence; PpALG3 TT is the P. pastoris ALG3 termination sequence; and pGAP is the P. pastoris GAPDH promoter.

FIG. 26 shows a map of plasmid pGLY5048 (pSH1275). Plasmid pGLY5048 (pSH1275) is an integration vector that targets the STE13 locus and contains expression cassettes encoding (1) the T. reesei α-1,2-mannosidase catalytic domain fused at the N-terminus to S. cerevisiae αMATpre signal peptide (aMATTrMan) to target the chimeric protein to the secretory pathway and secretion from the cell and (2) the P. pastoris URA5 gene or transcription unit.

FIG. 27 shows a map of plasmid pGLY5019 (pSH1246). Plasmid pGLY5019 (pSH1246) is an integration vector that targets the DAP2 locus and contains an expression cassette comprising a nucleic acid molecule encoding the Nourseothricin resistance (NAT^{R}) ORF operably linked to the Ashbya gossypii TEF1 promoter and A. gossypii TEF1 termination sequences flanked one side with the 5′ nucleotide sequence of the P. pastoris DAP2 gene and on the other side with the 3′ nucleotide sequence of the P. pastoris DAP2 gene.

FIG. 28 shows a map of plasmid pGLY5085 (pSH1312). Plasmid pGLY5085 (pSH1312) is a KINKO plasmid for introducing a second set of the genes involved in producing sialylated N-glycans into P. pastoris. The plasmid is similar to plasmid YGLY2456 except that the P. pastoris ARG1 gene has been replaced with an expression cassette encoding hygromycin resistance (HygR) and the plasmid targets the P. pastoris TRP5 locus. The six tandem cassettes are flanked on one side by a nucleic acid molecule comprising a nucleotide sequence from the 5′ region and ORF of the TRP5 gene ending at the stop codon followed by a P. pastoris ALG3 termination sequence and on the other side by a nucleic acid molecule comprising a nucleotide sequence from the 3′ region of the TRP5 gene.

FIG. 29 shows a map of plasmid pGLY5192. Plasmid pGLY5192 is an integration vector constructed to delete the ORF of the VPS10-1 gene to render the strain deficient in vacuolar sorting receptor (Vps10-1p) activity. The plasmid contains a nucleic acid molecule comprising the P. pastoris URA5 gene or transcription unit flanked by nucleic acid molecules comprising lacZ repeats which in turn is flanked on one side by a nucleic acid molecule comprising a nucleotide sequence from the 5′ region of the VPS10-1 gene and on the other side by a nucleic acid molecule comprising a nucleotide sequence from the 3′ region of the VPS10-1 gene.

FIG. 30 shows a map of plasmid pGLY3673. Plasmid pGLY3673 is a KINKO integration vector that targets the PRO1 locus without disrupting expression of the locus and contains expression cassettes encoding the T. reesei α-1,2-mannosidase catalytic domain fused at the N-terminus to S. cerevisiae αMATpre signal peptide (aMATTrMan) to target the chimeric protein to the secretory pathway and secretion from the cell.

FIG. 31 shows a map of plasmid pGLY7603. Plasmid pGLY7603 is an integration plasmid that expresses the LmSTT3D and targets the VPS10-1 locus in P. pastoris. The expression cassette encoding the LmSTT3D comprises a nucleic acid molecule encoding the LmSTT3D ORF codon-optimized for optimal expression in P. operably linked at the 5′ end to a nucleic acid molecule that has the inducible P. pastoris AOX1 promoter sequence and at the 3′ end to a nucleic acid molecule that has the S. cerevisiae CYC transcription termination sequence and for selection, the plasmid contains a nucleic acid molecule comprising the P. pastoris URA5 gene or transcription unit flanked by nucleic acid molecules comprising lacZ repeats. Both cassettes are flanked on one side by a nucleic acid molecule comprising a nucleotide sequence from the 5′ region of the VPS10-1 gene and on the other side by a nucleic acid molecule comprising a nucleotide sequence from the 3′ region of the VPS10-1 gene.

FIG. 32 shows a map of plasmid pGLY3588. The plasmid is an integration plasmid that targets the AOX1 locus and contains a nucleic acid molecule comprising the P. pastoris URA5 gene or transcription unit (PpURA5) flanked by nucleic acid molecules comprising lacZ repeats (lacZ repeat) which in turn is flanked on one side by a nucleic acid molecule comprising a nucleotide sequence from the 5′ region of the AOX1 gene and on the other side by a nucleic acid molecule comprising a nucleotide sequence from the 3′ region of the AOX1 gene.

FIGS. 33A and 33B show the construction of strains YGLY21058 and YGLY16415 in Example 3.

FIG. 34 shows the construction of strains YGLY23560 and YGLY24005 in Example 4.

FIGS. 35A and 35B show the construction of strain YGLY23605 in Example 5.

FIG. 36 shows the construction of strains YGLY21080, YGLY21081, and YGLY21083 in Example 6.

FIG. 37A and FIG. 37B show[s] an analysis of N-glycosylated proinsulin analogue precursors produced in strain YGLY21058. FIG. 37A: The reduced 16.5% Tricine polyacrylamide gel shows that the analogue was N-glycosylated. The N-glycosylated proinsulin analogue precursor was purified from culture supernatant fluid, the N-glycans released by PNGase digestion, and the observed N-glycan composition of the analogue was about 75% A2 (bisialylated) (SEQ ID NO:282), about 16% was A1 (monosialylated), and about 5% was hybrid Man₅, as shown in FIG. 37B.

FIG. 38A and FIG. 38B shows an analysis of positive MALDI-TOF of the purified N-glycosylated proinsulin analogue precursor (FIG. 38A) and deglycosylated proinsulin analogue precursor (FIG. 38A). The N-linked glycoforms attached to proinsulin analogue precursor are annotated in FIG. 38A and corresponding structures are shown in FIG. 37B.

FIG. 39A shows an analysis of N-glycosylated proinsulin analogue produced in strain YGLY21058 and resolved into pools on a RESOURCE RPC column. Aliquots of various pooled fractions were analyzed by gel electrophoresis (FIG. 39B) and the N-glycan composition determined for N-glycosylated proinsulin analogues in pools 1, 2, and 3 (FIG. 39C).

FIG. 40A, FIG. 40B, FIG. 40C, and FIG. 40D show in vivo activity of insulin B:P28N des(B30) analogues with an N-glycan attached to position B28. C57BL/6 mice at 12 weeks of age were fasted two hours before dosed with insulin des(B30) analogues with GS2.1 or GS5.0 N-glycan compositions by s.c injection. The effect on blood glucose was determined as a function of time in the absence and presence of α-methylmannose. FIG. 40A: vehicle; FIG. 40B: GS5.0; FIG. 40C: GS2.1; and FIG. 40D: summarizes the results.

FIG. 41A, FIG. 41B, FIG. 41C, and FIG. 41D show an analysis of the production of various insulin precursor sequences that contain zero, one, two, or three N-glycans. Cell-free culture supernatant fluid was loaded in 4-20% gradient reducing acrylamide gels and processed in SDS-PAGE. Insulin analogue precursors were visualized by coomassie blue staining. FIG. 41A shows the results for samples 1-12; FIG. 41B shows the results for samples 13-24; FIG. 41C shows the results for samples 25-35; and FIG. 41D shows the characteristics of samples 1-35.

FIG. 42A, FIG. 42B, and FIG. 42C provide a schematic representation of the process for producing an N-glycosylated insulin analogue from pre-proinsulin analogue precursors comprising an N-terminal spacer. FIG. 41A shows a schematic representation of the pre-proinsulin analogue encoded by pGLY3462 and FIG. 42B shows a schematic representation of the pre-proinsulin analogue encoded pGLY92295, pGLY9311, and pGLY9312. FIG. 42C shows the conversion of N-glycosylated proinsulin analogue precursor to N-glycosylated DesB30 insulin analogue.

FIG. 43A and FIG. 43B provide a schematic representation of the process for producing an N-glycosylated insulin analogue from pre-proinsulin analogue precursors lacking an N-terminal spacer. FIG. 43A shows a schematic representation of pre-proinsulin analogue precursor encoded by pGLY7680, pGLY9290, and pGLY9310. FIG. 43B shows the conversion of N-glycosylated proinsulin analogue precursor to N-glycosylated DesB30 insulin analogue.

FIG. 44 shows the impact of charge and N-glycan on stability of insulin at low pH and 65° C. over a five hour time period. Fibrillation of N-glycosylated B:P28N desB30 insulin analogues comprising A2 N-glycans (GS6.0) or Man₃GlcNAc₂ N-glycans (GS2.1), or deglycosylated B:P28D desB30 insulin were compared to NOVOLIN. Solutions of targeted insulin forms (1 mg/ml) were transferred into 0.5 ml conical tubes prepared with 100 mM HCl, pH 2.0. Vials were placed in a PCR machine set at 65° C. Aliquots of the sample were measured by ThioT fluorescence at time points 0 hr and 5 hr using Tecan plate reader with fluorescence scan from 440 nm-500 nm.

FIG. 45 shows a map of plasmid pGLY6301. Plasmid pGLY6301 is an integration plasmid that expresses the LmSTT3D and targets the URA6 locus in P. pastoris. The expression cassette encoding the LmSTT3D comprises a nucleic acid molecule encoding the LmSTT3D ORF codon-optimized for optimal expression in P. operably linked at the 5′ end to a nucleic acid molecule that has the inducible P. pastoris AOX1 promoter sequence and at the 3′ end to a nucleic acid molecule that has the S. cerevisiae CYC transcription termination sequence and for selection, the plasmid contains a nucleic acid molecule comprising the S. cerevisiae ARR3 gene to confer arsenite resistance.

FIGS. 46A and 46B show the construction of strain YGLY26268 in Example 11.

FIG. 47 shows map of plasmid pGLY9316, which is a roll-in integration plasmid that targets the TRP2 or AOX1p loci, includes an empty expression cassette utilizing the S. cerevisiae alpha mating factor signal sequence.

FIG. 48 shows the construction of strain YGLY26580 in Example 11.

FIGS. 49A and 49B show the construction of strain YGLY26734 in Example 11.

FIG. 50 shows map of plasmid pGLY11099, which is a roll-in integration plasmid that targets the TRP2 or AOX1p loci, includes an expression cassette encoding an insulin precursor fusion protein comprising a S. cerevisiae alpha mating factor signal sequence and propeptide fused to an N-terminal spacer peptide fused to the human insulin B-chain with NGT(−2) tripeptide addition and a P28N substitution fused to a C-peptide consisting of the amino acid sequence AAK (SEQ ID NO:139) fused to the human insulin A-chain.

FIG. 51 shows a plasmid map of pGLY1162, which is a KINKO plasmid that integrates at the PRO1 locus to express AOX1p-driven Tr. Mannosidase I. The integration of pGLY1162 at the PRO1 locus does not lead to a genetic disruption of the PRO1 open reading frame and selection is by the URA5 cassette.

FIG. 52A shows the dosage of N-glycosylated insulin analogue 210-2-B that when administered subcutaneously (s.c.) to the fasted diabetic minipig produces an effect on blood glucose levels over time that is equivalent to the effect of RHI has on blood glucose levels when administered subcutaneously (s.c.) to the fasted diabetic minipig.

FIG. 52B shows a comparison of the effect of N-glycosylated insulin analogue 210-2-B (paucimannose linked to Asn residues at B-2 and B28) versus recombinant human insulin (RHI) on blood glucose levels over time when administered subcutaneously (s.c.) to the fasted normal minipig.

FIG. 53A shows the data shown in FIG. 52B replotted as change in blood glucose from baseline.

FIG. 53B shows the data shown in FIG. 52A replotted as change in blood glucose from baseline.

FIG. 54A shows the dosage of N-glycosylated insulin analogue 200-2-B that when administered subcutaneously (s.c.) to the fasted diabetic minipig produces an effect on blood glucose levels over time that is equivalent to the effect of RHI has on blood glucose levels when administered subcutaneously (s.c.) to the fasted diabetic minipig.

FIG. 54B shows a comparison of the effect of N-glycosylated insulin analogue 200-2-B (Man₅GlcNAc₂ linked to Asn residues at B-2 and B28) versus recombinant human insulin (RHI) on blood glucose levels over time when administered subcutaneously (s.c.) to the fasted normal minipig.

FIG. 55A shows the data shown in FIG. 54B replotted as change in blood glucose from baseline.

FIG. 55B shows the data shown in FIG. 54A replotted as change in blood glucose from baseline.

FIG. 56A shows an image of a Western blot that detects secreted insulin analogue precursor from K. lactis induced for recombinant protein expression.

FIG. 56B shows an image of a Western blot that detects secreted insulin analogue precursor from K. lactis induced for recombinant protein expression.

FIG. 57A shows the structure of a glycosylated insulin analogue GSCI-7 comprising a native human A-chain peptide connected to a native human B-chain peptide by a connecting peptide comprising two Man₅GlcNAc₂ N-glycans (SEQ ID NO:303).

FIG. 57B shows in vivo activity of GSCI-7 with an N-glycan attached to position B28. C57BL/6 mice at 12 weeks of age were fasted two hours before dosed with insulin des(B30) analogues with GS2.1 or GS5.0 N-glycan compositions by s.c injection. The affect on blood glucose was determined as a function of time in the absence and presence of α-methylmannose

### DETAILED DESCRIPTION OF THE INVENTION

The present invention provides glycosylated insulin or insulin analogue molecules, compositions and pharmaceutical formulations comprising glycosylated insulin or insulin analogue molecules, methods for producing the glycosylated insulin or insulin analogues, and methods for using the glycosylated insulin or insulin analogues. The compositions and formulations are useful in treatments and therapies for diabetes.

In one embodiment, the glycosylated insulin or insulin analogues are N-linked glycosylated insulin analogues that comprise one or more attachment groups, each comprising an N-glycan attached in a β1 linkage to the asparagine residue comprising the attachment site. When a nucleic acid molecule encoding an insulin analogue having at least one attachment group for N-linked glycosylation is expressed in a host cell capable of producing glycoproteins, the insulin analogue, both in its precursor form and mature form, will include at least one N-linked glycan thereon linked to the asparagine residue comprising the attachment group. In particular embodiments, the processing of the N-glycosylated insulin analogue precursor to an N-glycosylated insulin analogue heterodimers may result in the removal of one or two of the amino acid residues comprising a functional attachment group.

In another embodiment, the glycosylated insulin or insulin analogue is an N-glycan conjugate wherein an attachment group on an insulin or insulin analogue molecule is conjugated in vitro to an N-glycan or the insulin or insulin analogue molecule is synthesized in vitro to include an amino acid residue that is covalently linked to an N-glycan.

### In Vivo N-Glycosylation

In a composition comprising N-linked glycosylated insulin analogue molecules, the predominant N-glycan species in the composition will depend on the host cell used for expression of the N-glycosylated insulin analogue. For example, expression of a nucleic acid molecule encoding an insulin analogue comprising one or more attachment sites, e.g., N-linked glycosylation sites, in a mammalian host cell, e.g., Chinese Hamster Ovary (CHO) or mouse myeloma host cells, will produce N-linked glycosylated insulin analogues in which the glycosylation pattern is heterogeneous and typical for glycoproteins produced in the mammalian host cell. Currently, there are only a few mammalian host cells that have been genetically modified to have an N-linked glycosylation pattern that differs from the N-linked glycosylation pattern typical for the unmodified host cell ((See for example, U.S. Patent Publication No. 20040110704; Yamane-Ohnuki et al. (2004) Biotechnol Bioeng 87:614-22; EP 1176195; WO 03/035835; Shields et al. (2002) J. Biol. Chem. 277:26733-26740). While a composition of N-linked glycosylated insulin analogues, which have been produced in a mammalian host cell will comprise a heterogeneous pattern of N-glycosylation, in general, a particular glycoform will predominate.

Plant, filamentous fungus, yeast, algae, prokaryote and insect host cells produce glycoproteins with non-mammalian N-glycosylation patterns. However, these host cells, particularly yeast host cells, can all be genetically engineered to control the type of N-linked glycosylation patterns to not only be similar to the patterns observed in mammalian or human cells but also to control which particular N-glycan species will predominate in a composition of glycoproteins produced in a host cell. This has been achieved by removing unwanted glycosyltransferases from the host cells and introducing particular combinations of glycosidases and/or glycosyltransferases. For example, yeast host cells, which have been genetically engineered to lack the ability to produce a yeast glycosylation pattern of hypermannosylated N-glycans, e.g., the yeast host cell is genetically engineered to not display α1,6-mannosyltransferase activity with respect an N-glycan, have been further manipulated to include various combinations of mammalian glycosyltransferases. As shown herein, these yeast host cells, which produce glycoproteins in which particular N-glycan structures predominate, have been used to make N-linked glycosylated insulin analogues. These genetically engineered host cells provide the ability to control the N-glycosylation pattern of the glycoproteins produced in the host cell. Therefore, compositions of N-linked glycosylated insulin analogues can be provided wherein a particular N-glycan structure predominates. However, regardless of the host cell that is used to produce the N-linked glycosylated insulin analogue, in general, the minimal polysaccharide unit of any N-glycan species will be the Man₃GlcNAc₂ in which the GlcNAc residue at the reducing end is linked to an aspargine residue comprising an N-linked glycosylation site. However, in particular aspects, the host cell may further include recombinantly expressed enzymes that trim the N-glycan to a glycoform consisting of Man₂GlcNAc₂, ManGlcNAc₂, or GlcNAc or the N-glycans may be treated in vitro to produce a glycoform consisting of Man₂GlcNAc₂, ManGlcNAc₂, or GlcNAc.

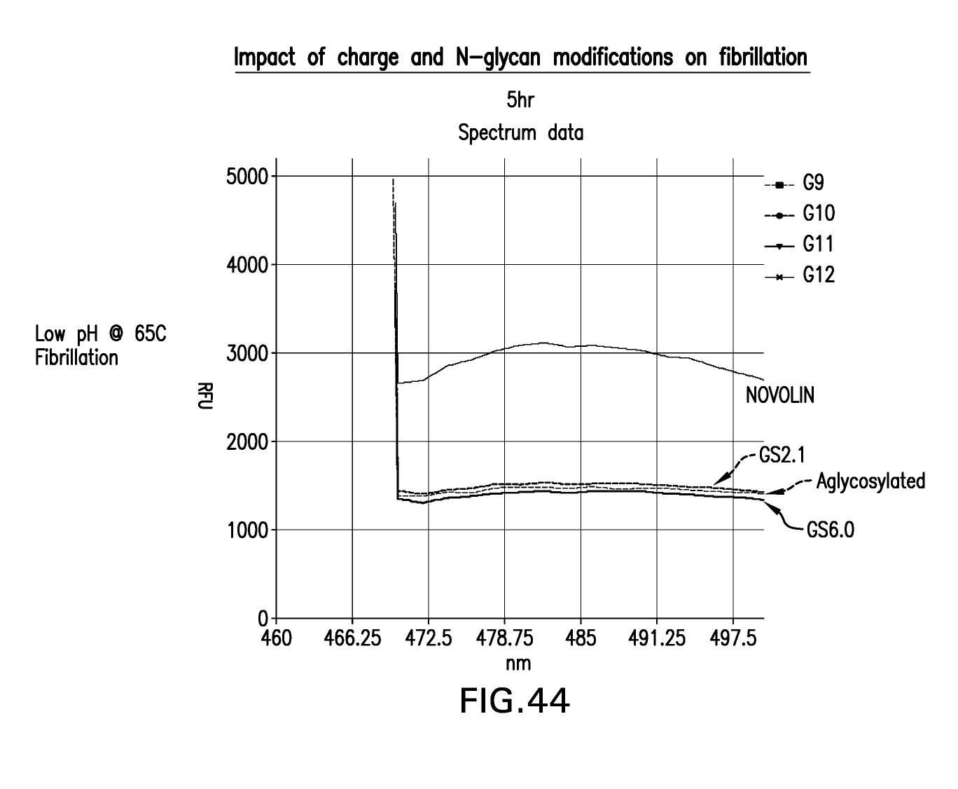

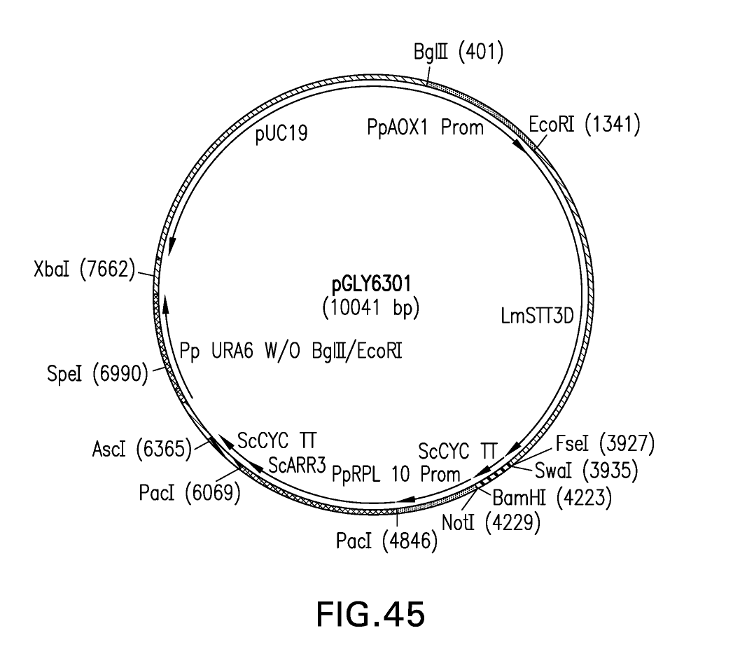

Insulin does not naturally contain an N-linked glycosylation site; therefore, in the present invention, the nucleic acid molecule encoding the insulin or insulin analogue is modified to introduce at least one N-linked glycosylation site (attachment site) into the nucleotide sequence to provide a nucleic acid molecule encoding an insulin analogue. An N-linked glycosylation site comprises the tri-amino acid sequence Asn-Xaa-(Ser/Thr) wherein Xaa is any amino acid except proline. The amino acid mutation and the particular N-linked glycan thereon may confer one or more beneficial properties to the N-glycosylated insulin analogue compared to a non-glycosylated N-glycosylated insulin analogue, including but not limited to, enhanced or extended pharmacokinetic (PK) properties, enhanced pharmacodynamic (PD) properties, reduced side effects such as hypoglycemia, enable the N-glycosylated insulin analogue to display glucose-sensitive activity, display a reduced affinity to the insulin-like growth factor 1 receptor (IGF 1R) compared to affinity to the insulin receptor (IR), display preferential binding to either the IR-A or IR-B, display an increased on-rate, decreased on-rate, and/or reduced off-rate to the insulin receptor, and/or altered route of delivery, for example oral, nasal, or pulmonary administration verses subcutaneous, intravenous, or intramuscular administration. For example, as shown in the examples and FIG. 44, N-glycosylated insulin analogues comprising an N-glycan have enhanced stability and a reduced tendency to form fibrils (fibrillation) induced at low pH and high temperature compared to native insulin and particular N-glycan structures appear to enable the glycosylated insulin analogue to have activity at the insulin receptor that is sensitive to or responsive to the concentration of glucose in the serum.

An N-linked N-glycan on an insulin analogue may confer one or more of the above attributes and may provide a significant improvement over current diabetes therapy. For example, particular N-linked N-glycans are known to alter the PK/PD properties of therapeutic proteins. Currently marketed insulin therapy consists of recombinant human insulin and mutated variants of human insulin called insulin analogues. These analogues exhibit altered in vitro and in vivo properties due to the combination of the amino acid mutation(s) and formulation buffers. The addition of an N-glycan to insulin adds another dimension for modulating insulin action in the body that is lacking in all current insulin therapies. Insulin conjugated to a saccharide or oligosaccharide moiety either directly or by means of polymeric or non-polymeric linker has been described previously, for example in U.S. Pat. No. 3,847,890; U.S. Pat. No. 7,317,000; Int. Pub. Nos. WO8100354; WO8401896; WO9010645; WO2004056311; WO2007047977; WO2010088294; and EP0119650). A feature of the glycosylated insulin analogues disclosed herein is that the N-glycan attached thereto is a natural structure. In embodiments in which the N-glycan is linked to an asparagine residue in vivo, the linkage is a natural chemical bond that can be produced in vivo by any organism with N-linked glycosylation capabilities.

For over three decades, insulin researchers have described attaching a saccharide to insulin using a chemical linker or ex vivo enzymatic reaction in an attempt to improve upon existing insulin therapy. The concept of chemical attachment of a sugar moiety to insulin was first introduced in 1979 by Michael Brownlee as a mechanism to modulate insulin bioavailability as a function of the physiological blood glucose level (Brownlee & Cerami, Science 206: 1190 (1979)). The major limitation of the initial proposal was toxicity of concanavalin A, to which the glycosylated insulin derivative interacted. There have been reports in the literature describing the presence of an O-linked mannose glycan on insulin produced in yeast, but this glycan was considered a contaminant (Kannan et al., Rapid Commun. Mass Spectrom. 23: 1035 (2009); International Publication Nos. WO9952934 and WO2009104199). Therefore, in one embodiment, the present invention provides N-glycosylated insulin or insulin analogues (either in the precursor form or mature form, in a heterodimer form, or in a single-chain chain form) to which at least one N-glycan is attached in vivo and wherein the N-glycan alters at least one therapeutic property of the N-glycosylated insulin analogue, for example, rendering the insulin or insulin analogue into a molecule that is has at least one modified pharmacokinetic (PK) and/or pharmacodynamic property (PD); for example, extended serum half-life, improved stability on solution, capable of being a glucose-regulated insulin, or capable of being able to target a particular receptor such as the asialoglycoprotein receptor (ASGPR) (Ashwell-Morell receptor) of the liver.

Currently, Escherichia coli, Saccharomyces cerevisiae, and Pichia pastoris are used to produce commercially available recombinant insulins and insulin analogues. Of these three organisms, only the yeasts Saccharomyces cerevisiae and Pichia pastoris have the innate ability to add an N-glycan to a protein. In general, N-glycosylation in yeast results in the production of glycoproteins in which the N-glycans thereon that have a fungal-type high mannose or hypermannosylated structure. For example, Glendorf et al., PLoS ONE 6(5) e20288 (2011) in a report on insulin receptor (IR) isoform-selective insulin analogues discloses construction of an analogue that had an asparagine residue substituted for the phenylalanine at position 25 of the B-chain, which was expressed in a Saccharomyces cerevisiae strain that produces glycoproteins with fungal-type N-glycans. The authors assumed the glycosylated analogues did not bind to the IR. When glycoproteins that include fungal high mannose or hypermannosylated structures are administered to a mammal or human, the glycoprotein is rapidly cleared from circulation and in some cases, may provoke an unwanted immune response. However, over the past decade yeast strains have been constructed in which the glycosylation pattern has been changed from a fungal type to a mammalian or human type. For example, using the glycoengineered Pichia pastoris strains as disclosed herein, the N-glycan composition of the glycoprotein can be pre-determined and controlled. Therefore, glycoprotein compositions can be produced in which a particular N-glycan is the predominant species (See for example, Hamilton et al., Science 313: 1441 (2006); Hamilton & Gerngross, Curr. Opin. Biotechnol. 18: 387 (2007); Li & d'Anjou, Curr. Opin. Biotechnol. 20: 678 (2009); Wildt & Gerngross, Nat. Rev. Microbiol. 3: 119 (2005). Thus, the glycoengineered yeast platform, is well suited for producing N-glycosylated insulin and insulin analogues. While N-glycosylated insulin may be expressed in mammalian cell culture, it currently appears to be an unfeasible means for recombinantly producing insulin since mammalian cell cultures routinely require the addition of insulin for optimal cell viability and fitness. Since insulin is metabolized in a normal mammalian cell fermentation process, the secreted N-glycosylated insulin analogue may likely be utilized by the cells resulting in reduced yield of the N-glycosylated insulin analogue. A further disadvantage to the use of mammalian cell culture is the current inability to modify or customize the glycan profile to produce compositions in a particular N-glycan is predominant (Sethuraman & Stadheim, Curr. Opin. Biotechnol. 17: 341 (2006)).

Recent reports describe the genetic engineering of prokaryotes to support protein glycosylation (Henderson, Isett, & Gerngross, Bioconjug Chem. 2011 Apr. 7; Pandhal, Ow, Noirel, & Wright, Biotechnol Bioeng. 2011 April; 108(4):902-12; Fisher et al., Appl Environ Microbiol. 2011 February; 77(3):871-81). Also, species of Archaea and other prokaryotes are reported to N-glycosylate proteins (Calo, Guan, & Eichler, Microb Biotechnol. 2011 Feb. 21). Thus, the N-linked glycosylated insulin analogues disclosed herein may be produced from prokaryotes genetically engineered to produce glycoproteins in which a particular N-glycan predominates.

There are many advantages to producing the N-glycosylated insulin analogues as described herein. Genetically engineered (or glycoengineered) Pichia pastoris provides the attractive properties of other yeast-based insulin production systems for insulin, including fermentability and yield. Genetic engineering allows for in vivo maturation of insulin precursor to eliminate process steps of enzymatic reactions and purifications. Pertaining to in vivo N-glycosylation, glycoengineered Pichia pastoris does not require the chemical synthesis or sourcing of the N-glycan moiety, as the yeast cell is the source of the glycan, which may result in improved yield and lower cost of goods. As described herein, glycoengineered Pichia pastoris strains can be selected that express N-glycosylated insulin with a particular predominant N-glycan structure, including the hybrid and complex N-glycan structures existing on human glycoproteins, which may be costly to synthesize using in vitro reactions and to purify. Moreover, a linker domain and non-natural glycans may in some cases be more immunogenic than an N-linked N-glycan and thereby reduce the effectiveness of the insulin therapy. Finally, an N-linked glycan structure on insulin may be further modified by enzymatic or chemical reactions to greatly expand the amount of N-glycan analogues that may be screened. As such, the optimal N-glycan may be identified more rapidly and with less cost than using purely synthetic strategies.

In general, the nucleic acid molecule encoding the N-glycosylated insulin analogue is mutated to encode at least one consensus N-linked glycosylation site motif (Asn-Xaa-Ser or Thr, wherein Xaa is any amino acid except for Pro), which when expressed in a host cell that is competent for N-linked glycosylation results in the production of an N-linked glycosylated insulin analogue. It is desirable that the host be capable of producing N-glycosylated insulin analogues wherein a particular N-glycan structure or glycoform predominates. A particular predominant N-glycan species may confer differentiated functional characteristics to the N-glycosylated insulin analogue such that the clinical profile is altered or improved. For example, particular N-glycan structures might result in differences in biological activity at the receptor level (i.e., increase and/or decrease binding at the IGF-1R, IR-A, IR-B) or N-linked glycosylation might influence alternative routes of clearance that result in glucose-responsive properties or differences in tissue distribution (e.g., targeting the liver) that result in a greater therapeutic index.

The amino acid substitutions of the currently marketed insulin analogues often focus on the carboxy-terminal end of the B-chain. Decades of research established mutations in this region retain binding to the insulin receptor (IR) but can have dramatic influences on the binding to insulin-like growth factor 1 receptor (IGF-1R). It is generally held that IGF-1R binding is undesirable for insulin (Zib & Raskin, Diabetes Obes. Metab 8: 611 (2006)). There are additional affects of mutations in this region such as solubility and oligomer formation that alter PK and PD properties of insulin analogues. For example, the insulin analogue insulin aspart (NOVOLOG) contains one amino acid substitution in the B-chain at position 28 in which the proline residue is substituted with aspartic acid. This substitution leads to the rapid onset and short acting profile of insulin aspart due to charge repulsion of the aspartic acid residue at B28 thereby preventing hexamer formation. Insulin aspart also has reduced IGF-1R binding. Data from the literature suggests insulin analogues with a more negative charge at the end of the B-chain leads to reduced IGF-1R binding (Zib & Raskin, op. cit.; Uchio et al., Adv. Drug Deliv. Rev. 35: 289 (1999)).

Therefore, in one embodiment of the N-glycosylated insulin analogues disclosed herein, the proline residue at position 28 of the B-chain is replaced with an asparagine residue (P28N substitution), which creates the tri-amino acid sequence of “NKT”. The NKT sequence provides a site for N-linked glycosylation when the N-glycosylated insulin analogue comprising the site is expressed in a host cell competent for producing glycoproteins that have N-glycans and in particular a host cell genetically engineered to produce glycoproteins that have predominantly a particular N-glycan species or glycoform.

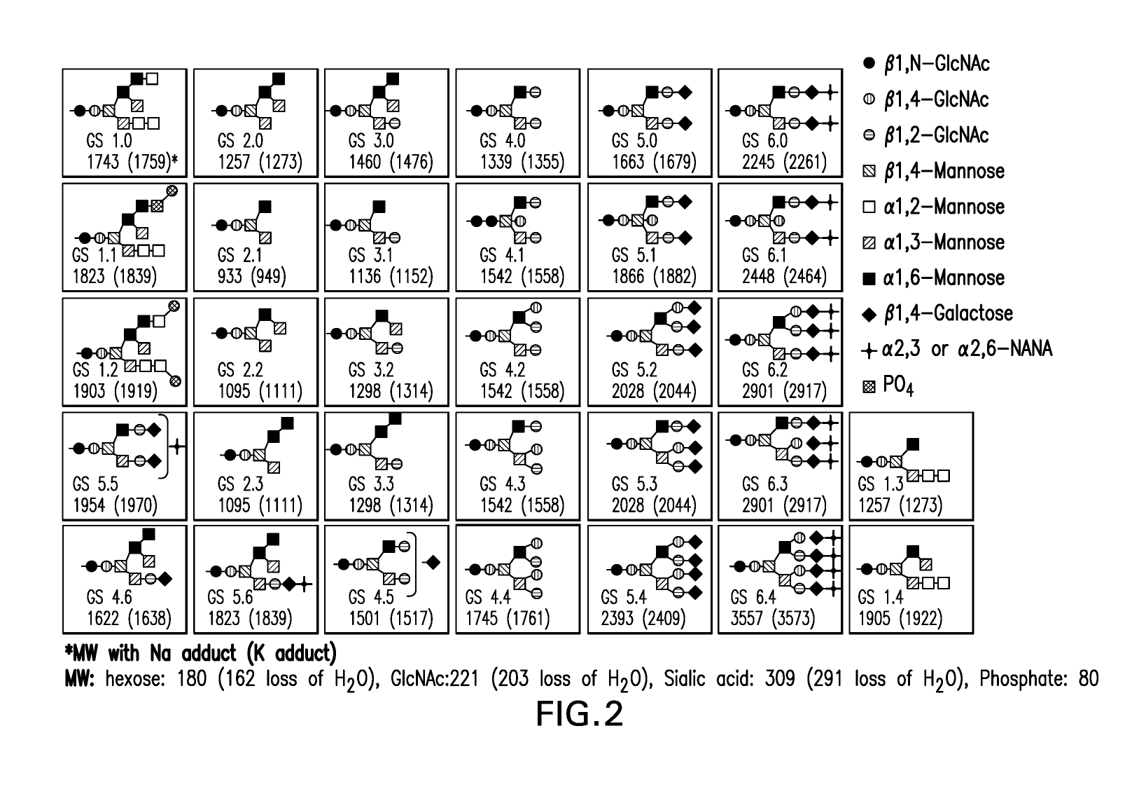

The addition of an N-linked N-glycan to the insulin analogue at the asparagine residue at position 28 of the B-chain provides an N-glycosylated insulin analogue that retains activity at the insulin receptor (IR). In addition, an N-linked N-glycan at position 28 of the B-chain adds an estimated mass of for example, about 910 Daltons in the case of Man₃GlcNAc₂ or about 2,222 Daltons in the case of NANA₂Gal₂GlcNAc₂Man₃GlcNAc₂ (See FIG. 2 for molecular weights for various N-glycan structures). The hydrodynamic volume of an N-glycan at position B28 may reduce hexamer formation. An N-glycan containing sialic acid (NANA) and its associated negative charge may further reduce interaction of the analogue with the IGF-1R, which would be desired from a clinical safety profile.

N-glycans are known to affect the pharmacokinetic properties of a glycoprotein. Proteins with sialic acid compositions tend to demonstrate an improved PK profile over the same protein without sialic acid. The improved PK profile may be due to reduced renal clearance at the glomerulus by the increased hydrodynamic volume of the protein and the increased charge repulsion with membranes at the site of filtration (Bork et al., J. Pharm. Sci. 98: 3499 (2009)). Furthermore, sialylated glycoproteins may demonstrate reduced hepatic clearance due to the masking of neutral glycans that interact with the asialoglycoprotein receptor (ASGPR) at the hepatocyte membrane. Therefore, sialic acid residues on an N-glycan at the position 28 of the B-chain may also provide a rapid-onset clinical profile to the analogue, since hexamer formation may be limited due to the negative charge, similar to insulin aspart. However, a sialylated N-glycosylated insulin analogue may not only exhibit rapid onset (reduced hexamer formation) similar to insulin aspart but may differ from insulin aspart by also exhibiting a longer duration of activity (improved PK profile). The transfer of additional sialic acid in the form of polysialic acid to the N-glycan would likely further extend the PK profile. The transfer of alternative glycans is clearly possible by transforming additional strains of glycoengineered Pichia.

### In Vitro Glycosylation

In another embodiment, the glycosylated insulin or insulin analogue is a conjugate wherein an attachment group is conjugated in vitro to an N-glycan or is synthesized in vitro to include an amino acid residue covalently linked to an N-glycan. In general, the attachment group or site and the N-glycan will include a functional moiety or group at the reducing end of the N-glycan that enables attachment of the N-glycan to the attachment group. The following table provides examples of useful attachment groups and activated N-glycans having a functional moiety or group that can couple the N-glycan to the attachment site.

| Attachment | Amino acid of | N-Glycan-functional group |
|---|---|---|
| Group | attachment group | for attachment |
| —NH₂ | N-terminal, Lys, Arg | N-Glycan-N-hydroxysuccinimide |
| | | N-Glycan-propionaldehyde |
| | | N-Glycan-aldehyde |
| —COOH | C-terminal, Asp, Glu | N-Glycan-hydrazide |
| —SH | Cys | N-Glycan-maleimide |
| | | N-Glycan-vinyl sulfone |
| | | N-Glycan-iodoacetamide |
| | | N-Glycan-bromoacetamide |
| | | N-Glycan-orthopyridyl dissulfide |
| Imidazole | His | N-Glycan-succinimidyl |
| ring | | N-Glycan-benzotriole |

In particular embodiments, the N-glycan is directly or indirectly conjugated to an attachment site in vitro by way of a linker or spacer. In particular embodiments, the linker or spacer comprises a chain of atoms from 1 to about 60, or 1 to 30 atoms or longer, 2 to 5 atoms, 2 to 10 atoms, 5 to 10 atoms, or 10 to 20 atoms long. In some embodiments, the chain atoms are all carbon atoms. In some embodiments, the chain atoms in the backbone of the linker or spacer are selected from the group consisting of C, O, N, and S. Chain atoms and linkers or spacers may be selected according to their expected solubility (hydrophilicity) so as to provide a more soluble conjugate. In some embodiments, the linker or spacer provides a functional group that is subject to cleavage by an enzyme or other catalyst or hydrolytic conditions found in the target tissue or organ or cell. In some embodiments, the length of the linker or spacer is long enough to reduce the potential for steric hindrance. If the linker or spacer is a covalent bond or a peptidyl bond and the insulin analogue is conjugated to a heterologous polypeptide, e.g., immunoglobulin, Fc fragment of an immunoglobulin, human serum albumin, the entire conjugate can be a fusion protein. Such peptidyl linkers may be any length. Exemplary linkers are from about 1 to 50 amino acids in length, 5 to 50, 3 to 5, 5 to 10, 5 to 15, or 10 to 30 amino acids in length.

In particular embodiments, the linker or spacer may be (i) one, two, three, or more unbranched alkane α, ω-dicarboxylic acid groups having one to seven methylene groups; (ii) one, two, three, or more amino acids; or, (iii) one, two, three, or more γ-aminobutanyl residues. In particular embodiments, the optional linker or spacer may be one, two, three, or more γ-glutamyl residues; one, two, three, or more β-alanyl residues; one, two, three, or more β-asparagyl residues; or one, two, three, or more glycyl residues.

In particular embodiments, the linker or spacer may be a covalent bond; a carbon atom; a heteroatom, an optionally substituted group selected from the group consisting of acyl, aliphatic, heteroaliphatic, aryl, heteroaryl, and heterocyclic; a bivalent, straight or branched, saturated or unsaturated, optionally substituted C1-30 hydrocarbon chain wherein one or more methylene units are optionally and independently replaced by —O—, —S—, —N(R)—, —C(O)—, C(O)O—, OC(O)—, —N(R)C(O)—, —C(O)N(R)—, —S(O)—, —S(O)2-, —N(R)SO2-, SO2N(R)—; each occurrence of R is independently hydrogen, a suitable protecting group, or an acyl moiety, arylalkyl moiety, aliphatic moiety, aryl moiety, heteroaryl moiety, or heteroaliphatic moiety.

Examples of linking moiety include but are not limited to γ-Glu (γE), γ-Glu-γ-Glu (γEγE), and polyethylene glycol.

In embodiments in which the attachment group comprises an amine, for example the amino group at N-terminus of the A-chain peptide (A1), the amino group at the N-terminus of the B-chain peptide (B1), the epsilon NH₂ group of a Lysine residue with the A-chain or B-chain peptide, or combinations thereof, provided are glycosylated insulin analogs comprising a native human insulin A-chain peptide (SEQ ID NO:33) or analogue thereof and a native insulin B-chain peptide (SEQ ID NO:25) or analogue thereof in which the N-terminus of the A-chain peptide or the N-terminus of the B-chain peptide or both the N-terminus and the A-chain peptide and the N-terminus of the B-chain peptide are directly or indirectly conjugated to an N-glycan.

Further provided are glycosylated insulin analogs comprising a native human insulin A-chain peptide or analogue thereof and a native insulin B-chain peptide or analogue thereof in which the epsilon NH₂ of the Lys at position 29 of the B-chain peptide, the N-terminus of the A-chain peptide and the epsilon NH₂ of the Lys at position 29 of the B-chain peptide, the N-terminus of the B-chain peptide and the epsilon NH₂ of the Lys at position 29 of the B-chain peptide, or both the N-terminus of the A-chain peptide and the N-terminus of the B-chain peptide and the epsilon NH₂ of the Lys at position 29 of the B-chain peptide are directly or indirectly conjugated to an N-glycan.

Further provided are glycosylated insulin glargine analogs comprising an A-chain peptide having the amino acid sequence shown in SEQ ID NO:34 and a B-chain peptide having the amino acid sequence shown in SEQ ID NO:27 in which the N-terminus of the A-chain peptide or the N-terminus of the B-chain peptide or both the N-terminus and the A-chain peptide and the N-terminus of the B-chain peptide are directly or indirectly conjugated to an N-glycan.

Further provided are N-glycosylated insulin glargine analogs comprising an A-chain peptide having the amino acid sequence shown in SEQ ID NO:34 and a B-chain peptide having the amino acid sequence shown in SEQ ID NO:27 in which the epsilon NH₂ of the Lys at position 29 of the B-chain peptide, the N-terminus of the A-chain peptide and the epsilon NH₂ of the Lys at position 29 of the B-chain peptide, the N-terminus of the B-chain peptide and the epsilon NH₂ of the Lys at position 29 of the B-chain peptide, or both the N-terminus of the A-chain peptide and the N-terminus of the B-chain peptide and the epsilon NH₂ of the Lys at position 29 of the B-chain peptide are directly or indirectly conjugated to an N-glycan.

In further embodiments, the glycosylated insulin analog comprises a native human insulin A-chain peptide and a B-chain peptide in which the Pro-Lys at positions 28-29 is replaced with Lys-Pro (insulin lispro, SEQ ID NO:298), a native human insulin A-chain peptide and a B-chain peptide in which the Pro at position 28 is replaced with an Asp residue (insulin aspart, SEQ ID NO:299), a B-chain peptide in which the Asn at position 3 is replaced with a Lys residue and the Lys at position 29 is replaced with a Glu residue (insulin glulisine, SEQ ID NO:300), a B-chain lacking the Thr at position 30 and in which the Lys at position 29 is conjugated to palmitic acid (insulin degludec, SEQ ID NO:301), or a B-chain lacking the Thr at position 30 and in which the Lys at position 29 is conjugated to myristic acid (insulin detemir, SEQ ID NO:302) and the N-terminus of the A-chain peptide or the N-terminus of the B-chain peptide or both the N-terminus and the A-chain peptide and the N-terminus of the B-chain peptide are directly or indirectly conjugated to an N-glycan.

Further provided are a glycosylated insulin analogs comprising a native insulin A chain and an insulin lispro B-chain peptide in which the epsilon NH₂ of the Lys at position 28 of the B-chain peptide, the N-terminus of the A-chain peptide and the epsilon NH₂ of the Lys at position 28 of the B-chain peptide, the N-terminus of the B-chain peptide and the epsilon NH₂ of the Lys at position 28 of the B-chain peptide, or both the N-terminus of the A-chain peptide and the N-terminus of the B-chain peptide and the epsilon NH₂ of the Lys at position 28 of the B-chain peptide are directly or indirectly conjugated to an N-glycan.

Further provided are a glycosylated insulin analogs comprising a native insulin A chain and an insulin aspart B-chain peptide in which the epsilon NH₂ of the Lys at position 29 of the B-chain peptide, the N-terminus of the A-chain peptide and the epsilon NH₂ of the Lys at position 29 of the B-chain peptide, the N-terminus of the B-chain peptide and the epsilon NH₂ of the Lys at position 29 of the B-chain peptide, or both the N-terminus of the A-chain peptide and the N-terminus of the B-chain peptide and the epsilon NH₂ of the Lys at position 29 of the B-chain peptide are directly or indirectly conjugated to an N-glycan.

Further provided are a glycosylated insulin analogs comprising a native insulin A chain and an insulin glulisine B-chain peptide in which the epsilon NH₂ of the Lys at position 3 of the B-chain peptide, the N-terminus of the A-chain peptide and the epsilon NH₂ of the Lys at position 3 of the B-chain peptide, the N-terminus of the B-chain peptide and the epsilon NH₂ of the Lys at position 3 of the B-chain peptide, or both the N-terminus of the A-chain peptide and the N-terminus of the B-chain peptide and the epsilon NH₂ of the Lys at position 3 of the B-chain peptide are directly or indirectly conjugated to an N-glycan.

In embodiments in which the attachment group comprises a Cys residue, the Cys residue is not any of the Cys residues at positions 6, 7, and 20 of the A-chain and positions 7 and 19 of the B-chain. In particular embodiments, the Cys residue will be at the N- and/or C-terminus of the A- and/or B-chain.

In vitro glycosylation of proteins and peptides is known in the art. For example, Yamamoto et al. in Tetrahedron Letters 45: 3287-3290 (2004) (the disclosure of which is incorporated herein by reference) discloses a method for in vitro synthesis of a glycopeptide in which a bromoacetyamidyl disialyl-undecasaccharide (NANA₂Gal₂GlcNAc₂Man₃GlcNac₂-NHCOCH₂Br was conjugated to the sulfhydryl group of cysteine residue in a peptide. Yamamoto et al. in Agnew. Chem. Int. Ed. 42: 2537-2540 (2003) (the disclosure of which is incorporated herein by reference) discloses solid-phase synthesis of sialylglycopeptides wherein an asparagine-linked disialyl-undecasaccharide Fmoc derivative (NANA₂Gal₂GlcNAc₂Man₃GlcNac₂-AsnFmoc) was incorporated into the peptide during synthesis of the peptide. Ito et at in U.S. Published Application No. 20100016547 and Andersen et al. in WO02055532 (the disclosures of which are incorporated herein by reference) discloses solid-phase synthesis of a variety of glycosylated GLP-1 analogues in which various asparagine-linked oligosaccharide or N-glycan structures are incorporated into the molecule during synthesis. Unverzagt (Agnew. Chem. Int. Ed. 36: 1989-1992 (1997)), Weiss & Unverzagt (Agnew. Chem. Int. Ed. 42: 4261-4263 (2003)), Eller et al. (Tetrahedron Letts. 51: 2648-2651 (2010), and Davis (Chem. Rev. 102: 579-601 (2002) all disclose methods for chemically synthesizing complex N-glycans in vitro.

These methods may be used to produce glycosylated insulin or insulin analogues having particular N-glycan structures covalently linked to an amino acid residue in the molecule. Thus, in particular embodiments, provided are glycosylated insulin or insulin analogues that have N-glycan structures as disclosed herein covalently linked to an amino acid or attachment group other than the asparagine residue comprising an attachment group for N-linked glycosylation. For example, in one embodiment, the N-glycan structures disclosed herein may be chemically synthesized to have an N-hydroxysuccinimide, acetaldehyde, or propionaldehyde group at the reducing end of the glycan molecule. The N-glycan may then be conjugated to an insulin or insulin analogue at the lysine residue at position B29 or at a lysine substituted for another amino acid elsewhere in the molecule. In another embodiment, the above insulin analogue or insulin may be conjugated at the histidine residue at B5 or a histidine substituted for an amino acid elsewhere in the molecule to an N-glycan structure as disclosed herein synthesized to have a succinimidyl or benzotriole group at the reducing end of the N-glycan molecule. In a further embodiment, an insulin analogue modified to include a cysteine residue may be conjugated to an N-glycan structure as disclosed herein synthesized to have a maleimide, vinyl sulfone, iodoacetamide, bromoacetamide, or orthopyridyl dissulfide group at the reducing end of the N-glycan molecule.

Wang in U.S. Pat. No. 7,807,405 (the disclosure of which is incorporated herein by reference) discloses an in vitro method for producing glycoproteins with homogenous N-glycosylation. The method entails treating a glycoprotein in vitro with endo-A, endo-F, endo-H, or endo-M to remove the N-glycan from the glycoprotein but leaving the GlcNAc residue at the reducing end attached to the asparagine residue in the glycoprotein and then reacting the glycoprotein with a sugar oxazoline having a particular glycan structure to reconstruct the N-linked N-glycan. The method enables the production of glycoprotein compositions wherein substantially all of the glycoproteins therein have the same N-glycan structures thereon. The methods disclosed therein may be used to produce various species of the N-glycosylated insulin analogues disclosed herein to provide compositions wherein the N-glycosylated insulin analogues therein are substantially homogenous for a particular glycoform.

### I. Protein Engineering of Insulin

Following initial reports of recombinant insulin expression in the 1980's, numerous studies were reported on the structure-activity relationship of mutant insulin proteins. The scientific literature has described the natural amino acid variations of insulin across species (See, for example, Conlon, Peptides 22: 1183 (2001)). Experiments using site-directed mutagenesis revealed substitutions with altered binding, physiochemical, or functional properties (Kohn et al., Peptides 28: 935 (2007); Kristensen et al., J. Biol. Chem. 272: 12978 (1997); Slieker et al., Diabetologia 40 Suppl 2, S54 (1997). Such information revealed the amino acids that are of critical importance for interacting with the insulin receptor are GlyA1, GlnA5, TyrA19, AsnA21, ValB12, TyrB16, GlyB23, PheB24, and PheB25 (Mayer et al., Biopolymers 88: 687 (2007)). As such, these residues may represent less attractive targets for modification by glycosylation. Although not exclusive, amino acid variations across species tend to dominate in a hypervariable region (A8-A10) and at the terminus of the B-chain (Conlon et al., op. cit.), and may represent attractive targets for glycosylation modification. Additional residues are substituted or added across species. Based on these data, amino acids in positions which a substitution results in no or only a modest change in activity of the molecule at the insulin receptor may modified to provide an attachment group for attachment of the glycan or oligosaccharide (e.g., modified to provide an N-linked glycosylation site). In particular embodiments, a glycosylated insulin analogue with a modest loss of activity at the insulin receptor may be advantageous for some application. For glycosylated insulin analogues in which the glycan confers an enhanced half-life, a loss of in vivo activity is recaptured in the longer half-life.

a. Protein Engineering for Glycosylation

The nucleic acid molecule encoding the insulin to be glycosylated in vivo is modified to contain an attachment group for N-linked glycosylation. The glycosylated insulin analogue may be a heterodimer or a single-chain insulin analogue in which a C-peptide or peptide domain from between 2 and 35 amino acid residues is between the B-chain peptide and A-chain peptide. The peptide domain may include one or more attachment sites for in vivo N-linked glycosylation. In particular embodiments, an attachment site for in vivo N-glycosylation may be placed at the N-terminus and/or C-terminus of the A- or B-chain, or both.

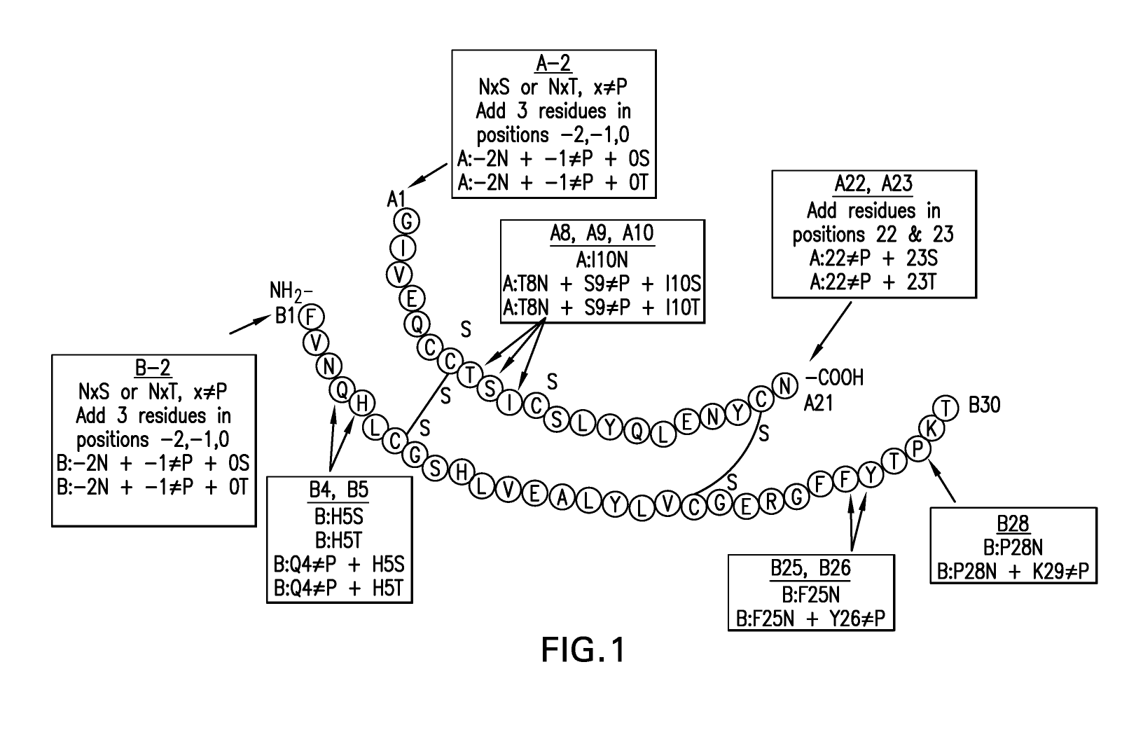

The examples herein illustrate production of an N-glycosylated insulin analogue in which an N-linked glycosylation site is introduced into the B-chain by replacing the proline residue at position 28 with an asparagine residue (P28N substitution). Additional N-linked glycosylation may occur at other positions in the B-chain, A-chain, or combinations thereof, for multiple N-glycan occupancy. Furthermore, amino acid substitutions to generate an N-linked consensus motif (attachment group) may be made to the amino acid sequence of native wild-type human insulin, to the amino acid sequence of any one of the currently available or described insulin analogues in the art, or to the amino acid sequence of any single-chain insulin. For example, an insulin analogue that includes the insulin glargine amino acid modifications of a glycine residue at position A21 and arginine residues at positions B31 and B32 may further include a B-chain P28N mutation in which the proline at position 28 is replaced with an asparagine to provide the N-linked glycosylation site having the amino acid sequence NKT. The extended PK properties of insulin glargine due to its insolubility at neutral pH may be maintained with the P28N substitution and the transfer of a neutral N-glycan to the asparagine. However, in particular embodiments, the glycosylated insulin glargine having the P28N substitution may have an N-glycan with an acidic charge may reduce the pI of the molecule to render it soluble at neutral pH. Such a molecule may require additional amino acid substitutions elsewhere in the molecule to re-gain neutral pH insolubility. FIG. 1 shows examples of several amino acid substitutions, single and double modifications, on the insulin molecule that would provide N-glycan attachment sites. The B-2, B3, B25, B28, A-2, A8, A10, and A21 positions represent sites in the insulin molecule in which an asparagine residue may be introduced to produce an N-linked glycosylation site while maintaining the ability of the molecule to bind the insulin receptor binding.

The following provides examples of insulin amino acid sequences that may be modified to include N-glycan motifs (attachment groups). Combinations of the following sequences may be applied to create N-glycosylated insulin analogue molecules with more than one N-glycosylation site or motif. Any substitutions that ablate the disulfide bond are not included below.

1. Single B-Chain Substitutions that Provide an N-Linked Glycosylation Site

B-chain H5S:
(SEQ ID NO: 42)
FVNQSLCGSHLVEALYLVCGERGFFYTPKT
B-chain H5T:
(SEQ ID NO: 43)
FVNQTLCGSHLVEALYLVCGERGFFYTPKT
B-chain F25N:
(SEQ ID NO: 44)
FVNQHLCGSHLVEALYLVCGERGFNYTPKT
B-chain P28N:
(SEQ ID NO: 26)
FVNQHLCGSHLVEALYLVCGERGFFYTNKT

2. Single A-Chain Substitutions that Provide an N-Linked Glycosylation Site

(SEQ ID NO: 45)
A-chain I10N: GIVEQCCTSNCSLYQLENYCN

3. Double B-Chain Modifications that Provide an N-Linked Glycosylation Site

B-chain substitutions to N: All positions except N3, H5, C7, L17, C19, T27
B-chain substitutions to S: All positions except C7, S9, C19, E21, K29
B-chain substitutions to T: All positions except C7, S9, C19, E21, T27, K29, T30
B-chain additions: The tripeptide NXS or NXT at the N-terminus of the B-chain (positions −2, −1, and 0, respectively) wherein F is position 1; S31 or T31 when the amino acid at position 29 is N and the amino acid at position 30 is not P; S32 or T32 when the amino acid at position 30 is N and the amino acid at position 31 is not P; any residue at position 0 except P when the amino acid at position 1 is S or T and at position −1 is N.

4. Double A-Chain Modifications that Provide an N-Linked Glycosylation Site

A-chain substitutions to N: All positions except E4, Q5, C6, C7, S9, C11, N18, C20, N21
A-chain substitutions to S: All positions except C6, C7, T8, S9, C11, S12, L13, C20
A-chain substitutions to T: All positions except C6, C7, T8, S9, C11, L13, C20
A-chain additions: The tripeptide NXS or NXT at the N-terminus of the A-chain (positions −2, −1, and 0, respectively) wherein G is position 1; S23 or T23 when the amino acid at position 21 is N and the amino acid at position 22 is not P; any residue at position 0 except P when the amino acid at position 1 is S or T and at position −1 is N.

The N-glycosylated insulin analogues may comprise any combination of substitutions and/or double modifications of the A-chain peptide, B-chain peptide, or both the A-chain peptide and B-chain peptide. Therefore, the N-glycosylated insulin analogues may comprise any combination of the N substitutions, S substitutions, T substitutions, and additions that results in insulin analogues that have a consensus N-linked glycosylation site or motif. Thus, in further embodiments, the N-glycosylated insulin analogues may include any combination of A-chain peptide and/or B-chain peptide substitutions and/or modifications to generate insulin analogues comprising one or more N-linked glycosylation sites. In further embodiments, the N-glycosylated insulin analogues do not include substitutions in positions A1, A2, A3, B6, B8, B11, B12 2B3, or B24 without further substitutions that improve insulin receptor binding activity.

5. Addition of N-Glycosylated Peptide Domains to B-Chain or A-Chain

Insulin glargine is an example of an insulin analogue that contains additional amino acids and still retains activity: it contains two additional arginine residues at the C-terminal end of the B-chain peptide. This suggests adding other peptide sequences at the N- and/or C-termini of B- and A-chain peptides may also yield insulin molecules that have activity at the insulin receptor. Thus, further included are N-glycosylated insulin analogues that have one, two, or more amino acids to the ends of either the B-chain or A-chain, or both. The addition of three amino acids to the N- or C-termini of the B-chain and/or A-chain that consist of the Asn-Xaa-(Ser/Thr) motif (attachment group), wherein Xaa is any amino acid except proline, and thus provides the recognition signal for the transfer of an N-glycan to the molecule. Additional sequences may be fused to insulin, and this may be accomplished using artificial or natural peptide or protein sequences, fusions with human proteins such as human serum albumin or Fc fragments, or fusions with proteins that contain N-glycosylation motifs. The protein fusions may be full or partial proteins that also contain attachment groups. For example, partial sequences from human NCAM that may enable transfer of polysialic acid to the glycosylated insulin analogue. An insulin analogue precursor that included a partial IG5-FN1 subdomain of NCAM in the C-peptide of the insulin analogue precursor which is removable by endoprotease processing in vitro may result in polysialylation at P28N of the B-chain or N21 of the A-chain peptide. The NCAM sequence would be excluded from glycosylated insulin analogue after endoprotease processing with trypsin or endopeptidase LysC.

### II. Glycodesign

The majority of therapeutic glycoproteins are currently produced in mammalian cell systems. Typically, N-glycans from mammalian cells are of complex structures that may be composed of mannose (Man), N-acetylglucosamine (GlcNAc), galactose (Gal), N-acetylneuraminic acid (NANA), N-glycolylneuraminic acid (NGNA), fucose (Fuc), and N-acetylgalactosamine (GalNAc).

The attachment of N-glycans may affect the PK and PD properties of insulin. As shown in the examples, when an N-glycosylated des(B30) insulin analogue having predominantly sialic acid-terminated N-glycans was compared to human des(B30) insulin (NOVOLIN modified to be des(B30)), the PK profile of the sialic acid-terminated N-linked glycosylated des(B30) insulin analogue was improved relative to the modified NOVOLIN and an N-glycosylated des(B30) insulin analogue having predominantly galactose-terminated N-glycans. The sialic acid-terminated N-linked glycosylated des(B30) insulin analogue also demonstrated reduced binding to the insulin growth factor receptor (IGF-1R). Both N-linked glycosylated des(B30) insulin analogues retained in vivo glucose reduction activities while specific attributes were modulated by the particular N-glycan structure.

a. N-Glycan Structures

FIG. 2 shows a non-limiting example of some of the N-glycan structures that may be generated with glycoengineered Pichia and which may be attached at the reducing end to an asparagine residue comprising attachment group in a β1 linkage. Any one of these glycoforms may be added to an insulin analogue comprising an attachment group. Many of the glycoforms shown may be produced in host cells genetically engineered to produce glycoproteins in which particular N-glycan structures predominate. However, for other glycoforms, additional genetic alterations, process changes, purification schemes, and/or in vitro enzymatic reactions in vitro may be used generate the N-glycosylated insulin analogues with the desired dominant glycoform. The group of glycoforms listed in FIG. 2 is not all-inclusive. Additional glycans may be synthesized in glycoengineered Pichia, such as polysialic acid, polylactosamine, sialylated Lewis X, GalNAc, fucose, glucose, and others. The structures shown in FIG. 2 may also be conjugated to an attachment group in vitro.

Therefore, in particular embodiments, the glycosylated insulin analogue disclosed herein includes one or more attachment groups for in vivo or in vitro glycosylation covalently linked to the GlcNAc residue at the reducing end of an oligosaccharide or glycan. Thus, provided are glycosylated insulin analogues having the having the formula

INSL-[X-R]ₙ

Wherein INSL is an insulin or insulin analogue molecule comprising an A-chain peptide, a B-chain peptide, three disulfide bonds, and one or more attachment groups (e.g., 1-10, or 1-5, or 1-2 attachment groups); n is an integer selected from 1-10, or 1-5, or 1-2, the integer value corresponding to the number of attachment groups in INSL; X is optionally a linker or spacer comprising one ore more amino acids or amino acid derivatives, a nonpeptide moiety, or both covalently linked to an attachment group or absent and in which each occurrence of the linker or spacer is independent of any other occurrence of linker or spacer; and R is an N-glycan structure linked at its reducing end to the attachment group or to the linker or spacer wherein each occurrence of R is the same or independently a particular N-glycan. The attachment group may be an Asn residue for in vivo N-glycosylation or NH₂, COOH, SH, or imidizole ring of His for in vitro glycosylation. In particular embodiments, the N-glycan is selected from structures 1 through 106 shown below.

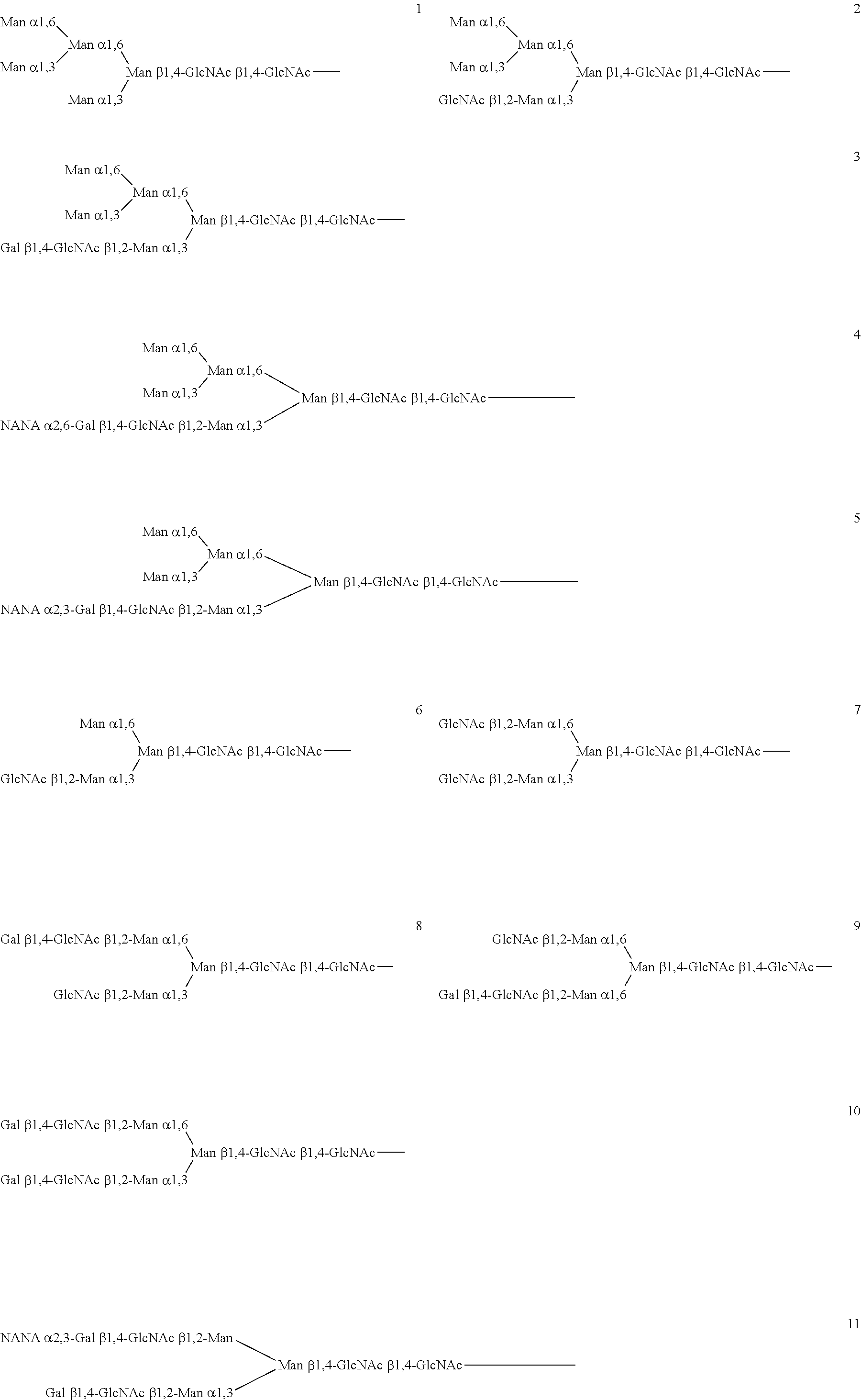

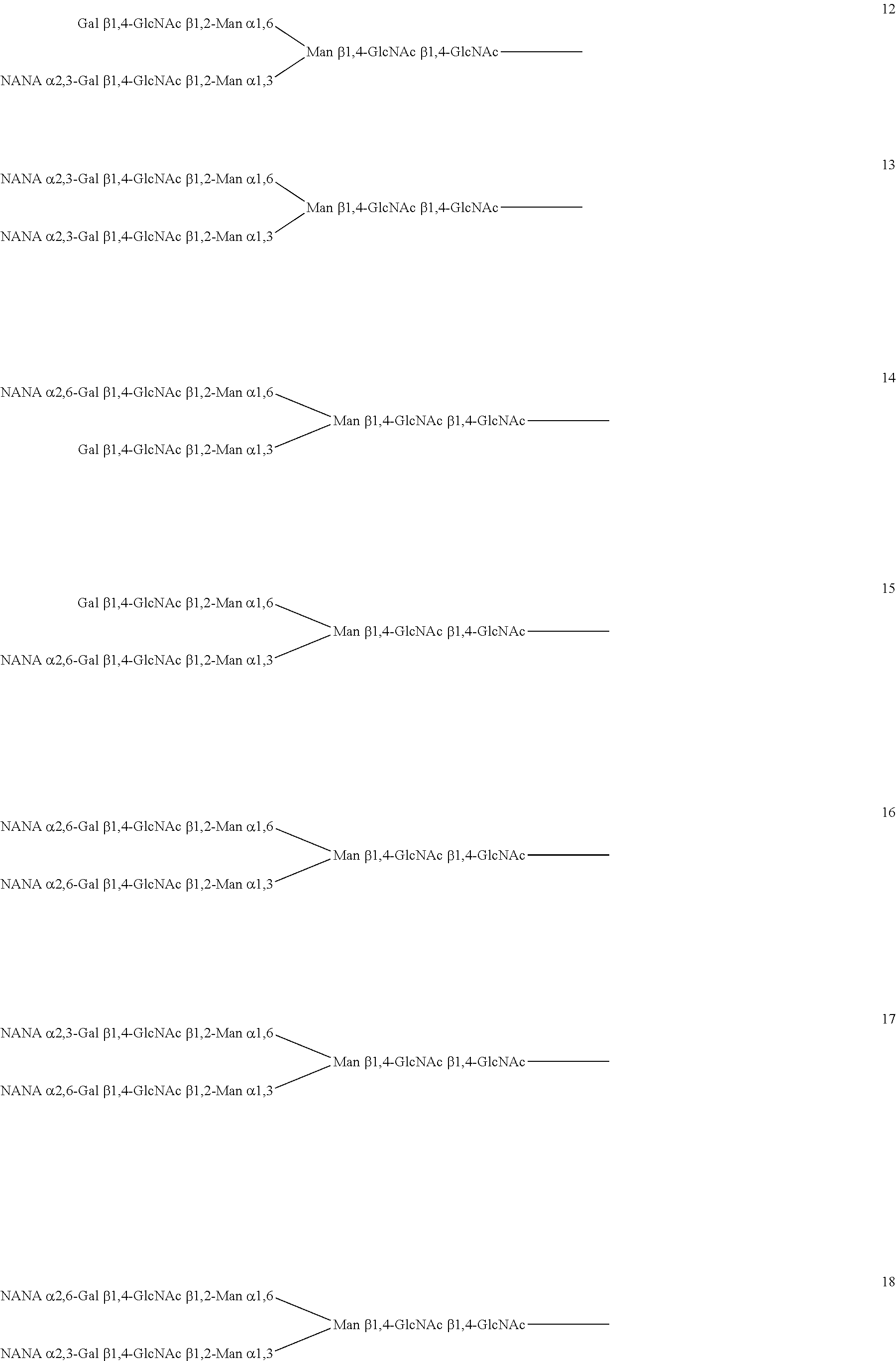

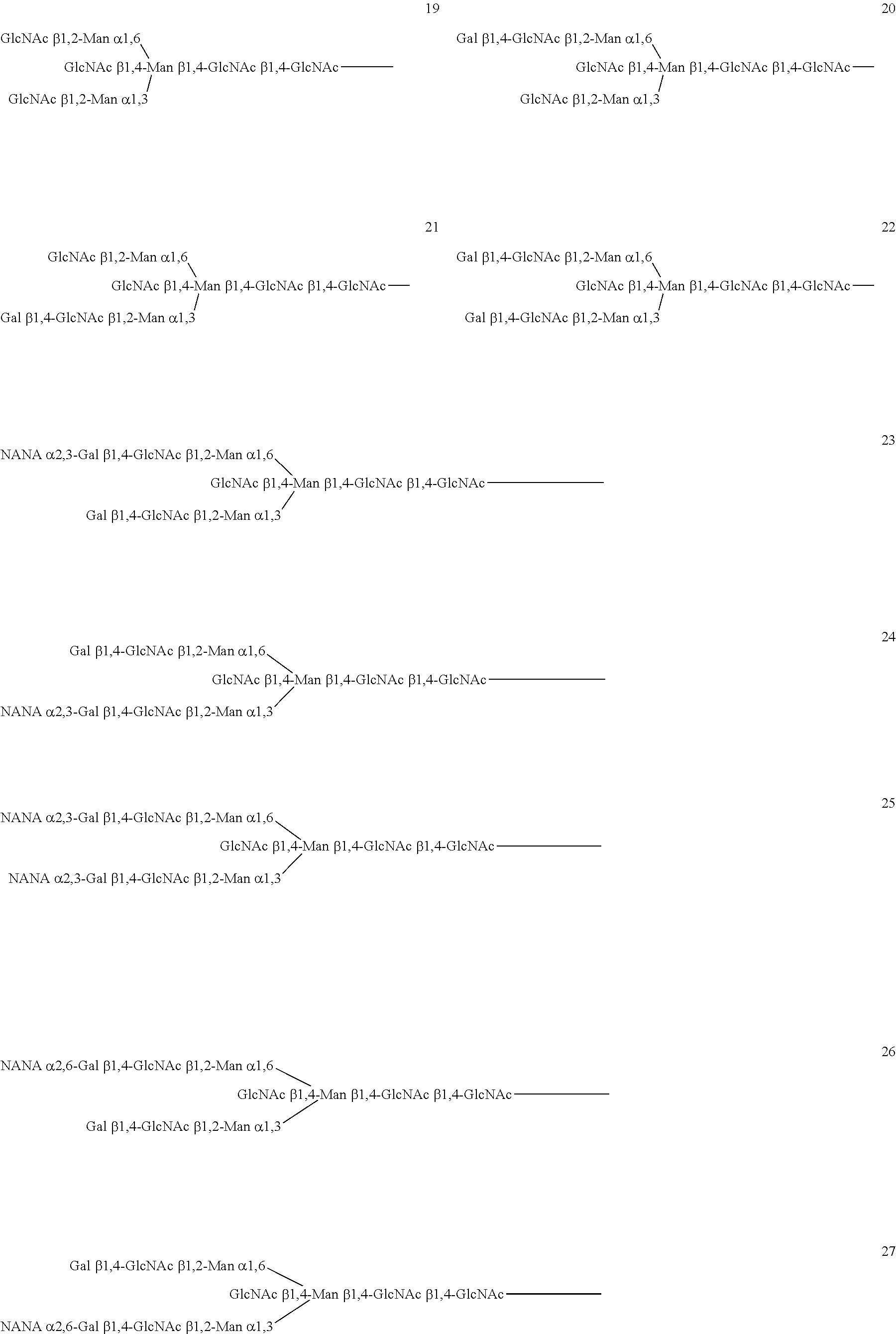

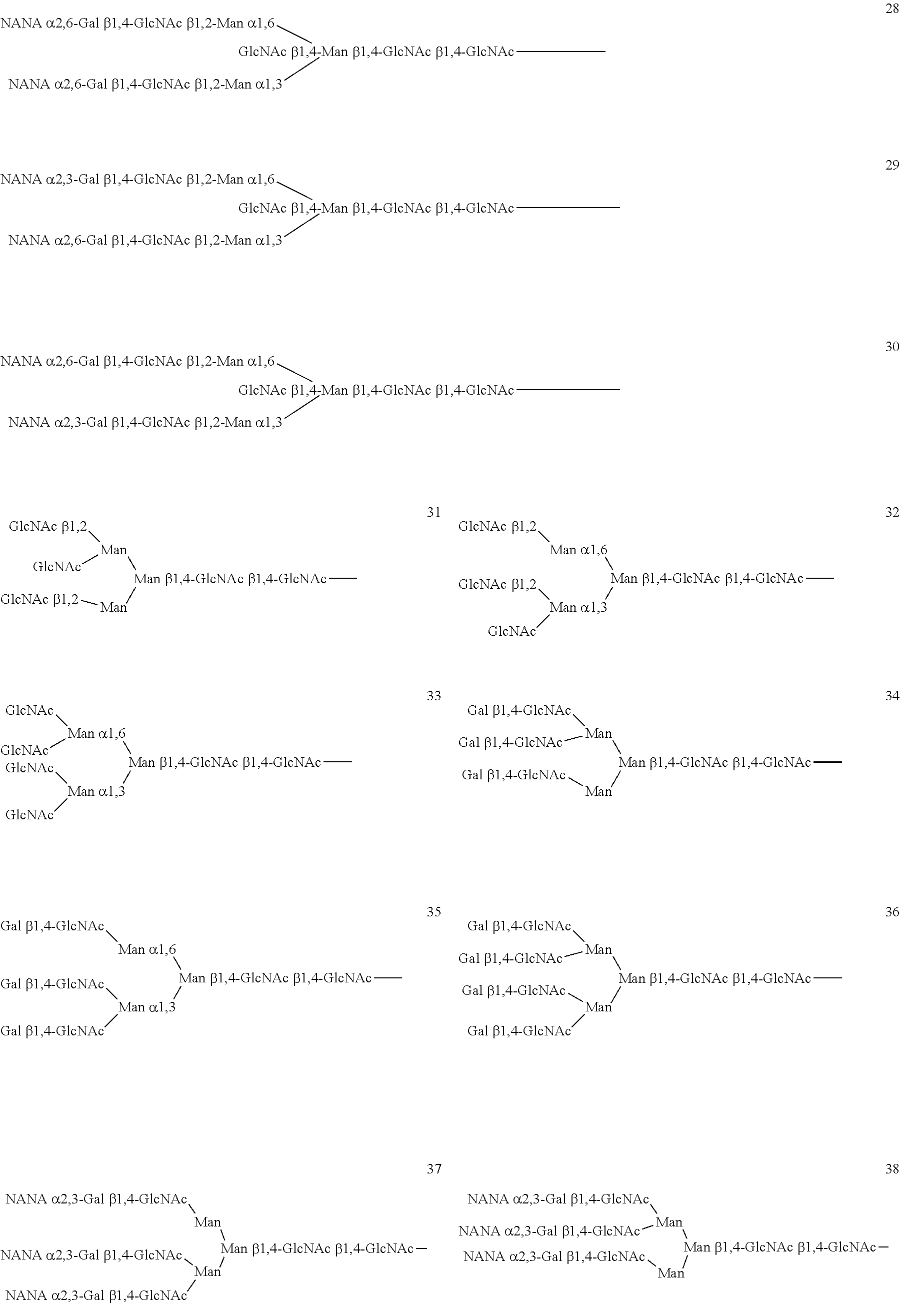

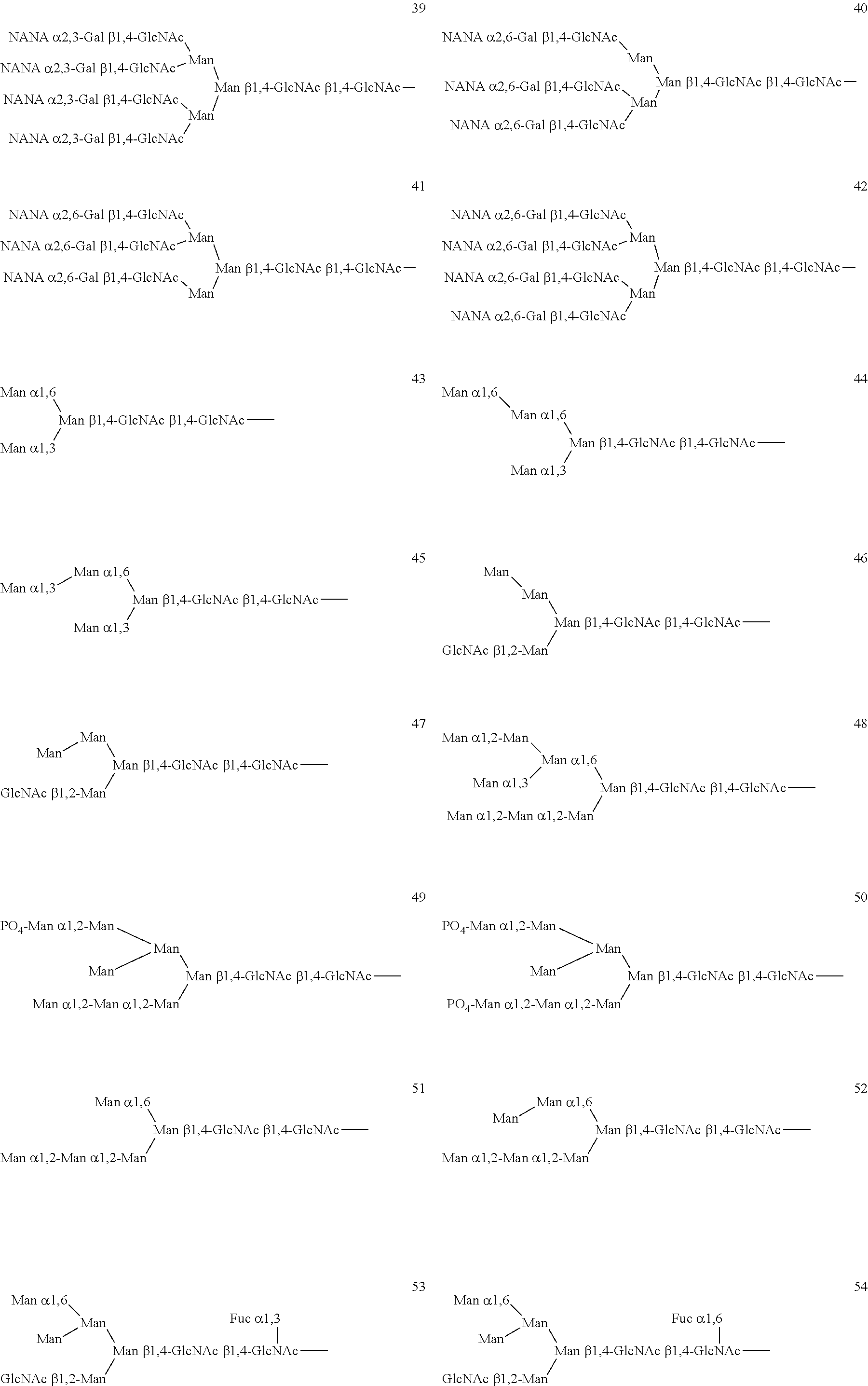

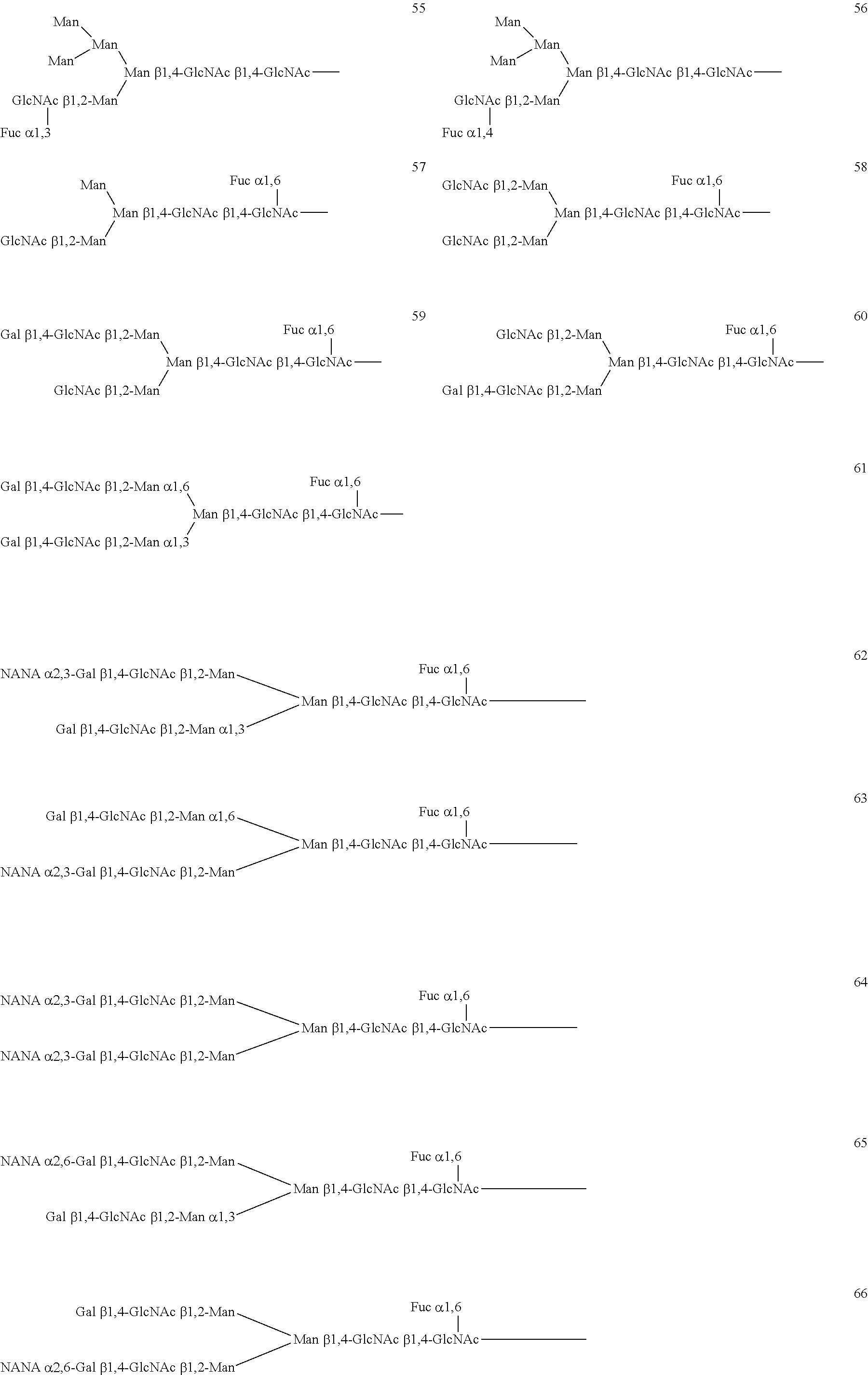

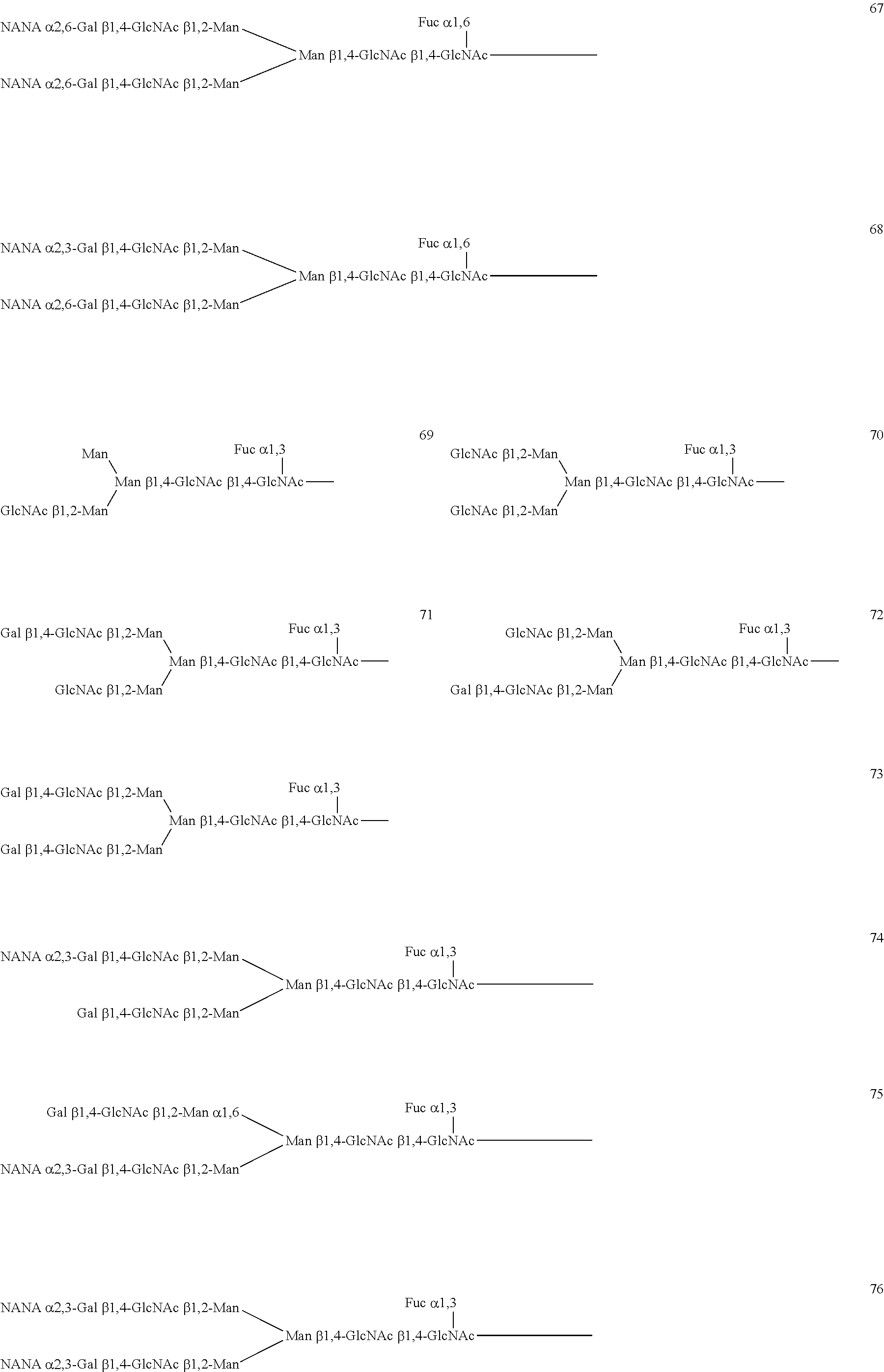

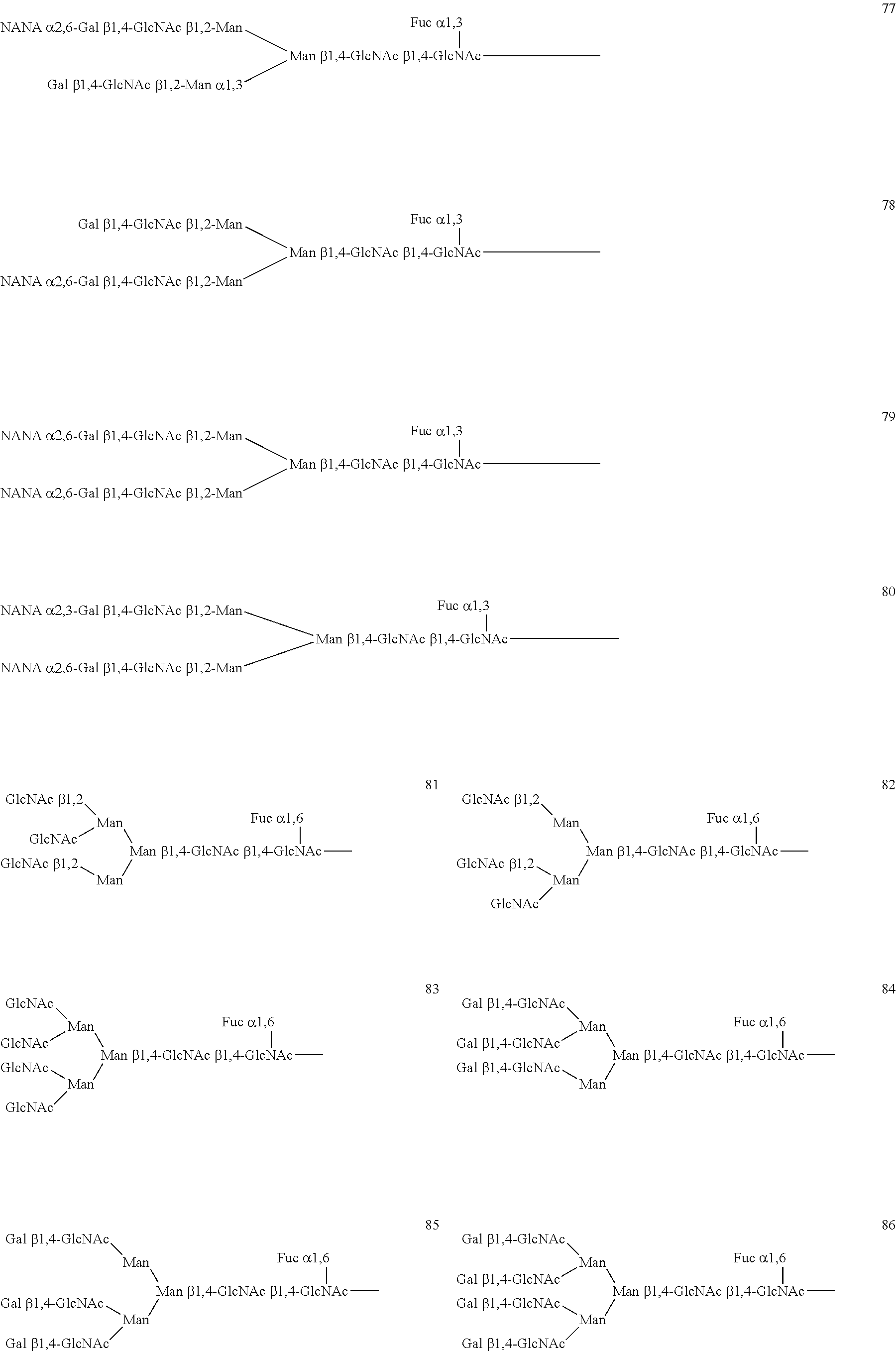

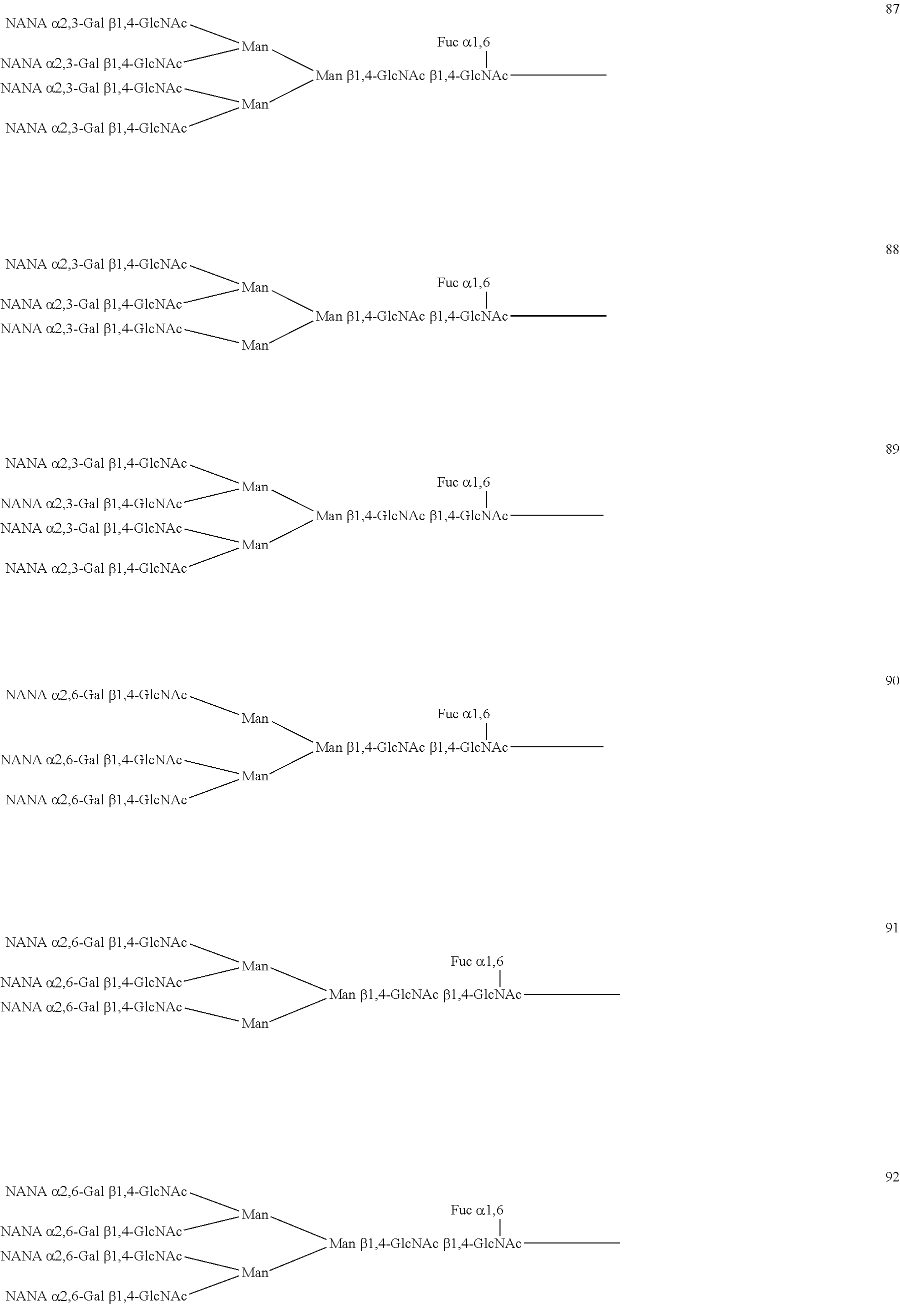

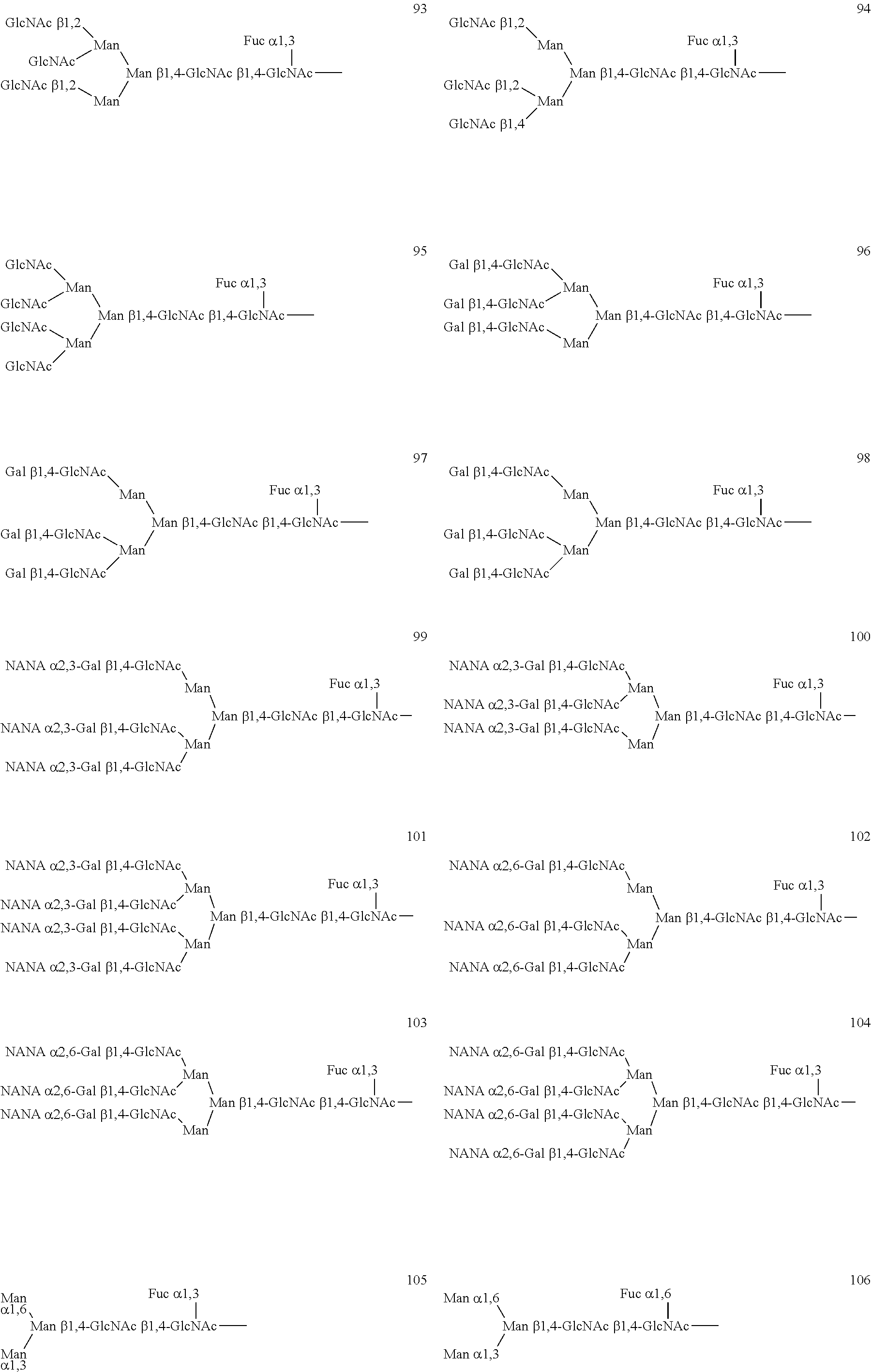

In particular embodiments, compositions or formulations are provided in which the glycosylated insulin or insulin analogues therein have the formula

INSL-[X-R]ₙ

Wherein INSL is an insulin or insulin analogue molecule comprising an A-chain peptide, a B-chain peptide, three disulfide bonds, and one or more attachment groups (e.g., 1-10, or 1-5, or 1-2 attachment groups); n is an integer selected from 1-10, or 1-5, or 1-2, the integer value corresponding to the number of attachment groups in INSL; X is optionally a linker or spacer comprising one ore more amino acids or amino acid derivatives, a nonpeptide moiety, or both covalently linked to an attachment group or absent and in which each occurrence of the linker or spacer is independent of any other occurrence of linker or spacer; and R is an N-glycan structure linked at its reducing end to the attachment group or to the linker or spacer wherein each occurrence of R is the same or independently a particular N-glycan, and a pharmaceutically acceptable carrier. The attachment group may be an Asn residue for in vivo N-glycosylation or NH₂, COOH, SH, or imidizole ring of His for in vitro glycosylation. In particular embodiments, the N-glycan is selected from structures 1 through 106. The compositions and formulations of comprise a pharmaceutically acceptable carrier, salt, or combination thereof.

In particular aspects, at least 70%, 75%, 80%, 85%, 90%, 95%, 96%, 97%, 98%, 99%, or 100% of the insulin or insulin analogues in the composition or formulation are glycosylated. In general, at least one N-glycan species selected from structures 1 through 106 in the composition or formulation will be predominant or predominate. In further aspects, at least 80% of the insulin or insulin analogues in the composition or formulation are glycosylated. In general, at least one N-glycan species selected from structures 1 through 106 in the composition or formulation will be predominant or predominate. In further aspects, at least 90% of the insulin or insulin analogues in the composition or formulation are glycosylated. In general, at least one N-glycan species selected from structures 1 through 106 in the composition or formulation will be predominant or predominate. In further aspects, at least 95% of the insulin or insulin analogues in the composition or formulation are glycosylated. In general, at least one N-glycan species selected from structures 1 through 106 in the composition or formulation will be predominant or predominate. In further aspects, at least 98% of the insulin or insulin analogues in the composition or formulation are glycosylated. In general, at least one N-glycan species selected from structures 1 through 106 in the composition or formulation will be predominant or predominate. In further aspects, at least 99% of the insulin or insulin analogues in the composition or formulation are glycosylated. In general, at least one N-glycan species selected from structures 1 through 106 in the composition or formulation will be predominant or predominate.

In particular aspects, about 30 mole % to about 100 mole % of the total N-glycans in the composition or formulation will consist of an N-glycan species selected from structures 1 through 106. In further aspects, between 30 mole % and 100 mole % of the total N-glycans in the composition or formulation will consist of an N-glycan species selected from structures 1 through 106. In further aspects, between 30 mole % and 80 mole % of the total N-glycans in the composition or formulation will consist of an N-glycan species selected from structures 1 through 106. In further aspects, between 50 mole % and 100 mole % of the total N-glycans in the composition or formulation will consist of an N-glycan species selected from structures 1 through 106.

Further, in particular compositions and formulations, about 30 mole of the total N-glycans in the composition or formulation will consist of an N-glycan species selected from structures 1 through 106. In a further aspect, about 40 mole % of the total N-glycans in the composition or formulation will consist of an N-glycan species selected from structures 1 through 106. In a further aspect, about 50 mole % of the total N-glycans in the composition or formulation will consist of an N-glycan species selected from structures 1 through 106. In a further aspect, about 60 mole % of the total N-glycans in the composition or formulation will consist of an N-glycan species selected from structures 1 through 106. In a further aspect, about 70 mole % of the total N-glycans in the composition or formulation will consist of an N-glycan species selected from structures 1 through 106. In a further aspect, about 80 mole % of the total N-glycans in the composition or formulation will consist of an N-glycan species selected from structures 1 through 106. In a further aspect, about 85 mole % of the total N-glycans in the composition or formulation will consist of an N-glycan species selected from structures 1 through 106. In a further e aspect, about 90 mole % of the total N-glycans in the composition or formulation will consist of an N-glycan species selected from structures 1 through 106. In a further aspect, about 95 mole % of the total N-glycans in the composition or formulation will consist of an N-glycan species selected from structures 1 through 106. In a further aspect, about 98 mole % of the total N-glycans in the composition or formulation will consist of an N-glycan species selected from structures 1 through 106. In a further aspect, about 99 mole % of the total N-glycans in the composition or formulation will consist of an N-glycan species selected from structures 1 through 106. In a further aspect, about 100 mole % of the total N-glycans in the composition or formulation will consist of an N-glycan species selected from structures 1 through 106.

In particular embodiments, the heterodimer or single-chain N-glycosylated insulin analogue comprises at least one asparagine (Asn or N) residue covalently linked to an N-glycan. Thus, in further embodiments, the heterodimer or single-chain N-glycosylated insulin analogue comprises any combination of A- and B-chain peptides having an amino acid sequence selected from the group of sequences shown by SEQ ID NOs:162 to 254 and 316 to 337 below or in combination with a native A- or B-chain provided that at least one asparagine residue in the heterodimer or single-chain insulin analogue is attached to an N-glycan. In further embodiments, the heterodimer N-glycosylated insulin analogue consists of any combination of A- and B-chain peptides having an amino acid sequence selected from the group of sequences shown by SEQ ID NOs:162 to 254 and 316 to 337 below or in combination with a native A- or B-chain provided that at least one of asparagine residue in the heterodimer or single-chain insulin analogue is attached to an N-glycan. Further provided are compositions and formulations of the above comprising a pharmaceutically acceptable carrier, salt, or combination thereof.

(SEQ INO: 162)
GIVEQCCN*SX1CSLYQLENYCN
(SEQ INO: 252)
GIVEQCCTSN*CSLYQLENYCN
(SEQ INO: 163)
GIVEQCCTSICSLYQLENYCN*
(SEQ INO: 164)
GIVEQCCTSN*CSLYQLENYCN*
(SEQ INO: 165)
GIVEQCCN*SX1CSLYQLENYCN*
(SEQ INO: 166)
N*X2X1GIVEQCCTSICSLYQLENYCN
(SEQ INO: 167)
N*X2X1GIVEQCCN*SX1CSLYQLENYCN
(SEQ INO: 168)
N*X2X1GIVEQCCTSN*CSLYQLENYCN
(SEQ INO: 169)
N*X2X1GIVEQCCTSICSLYQLENYCN*
(SEQ INO: 170)
N*X2X1GIVEQCCTSN*CSLYQLENYCN*
(SEQ INO: 171)
N*X2X1GIVEQCCN*SX1CSLYQLENYCN*
(SEQ INO: 172)
N*X2X1GIVEQCCTSICSLYQLENYCG
(SEQ INO: 173)
N*X2X1GIVEQCCN*SX1CSLYQLENYCG
(SEQ INO: 174)
N*X2X1GIVEQCCTSN*CSLYQLENYCG
(SEQ INO: 175)
GIVEQCCN*SX1CSLYQLENYCG
(SEQ INO: 176)
GIVEQCCTSN*CSLYQLENYCG
(SEQ INO: 316)
GIVEQCCTSN*CSLYQLENYCG
(SEQ INO: 317)
GIVEQCCN*SSCSLYQLENYCG
(SEQ INO: 318)
GIVEQCCN*RSCSLYQLENYCG

Wherein in the preceding A-chain sequences X1 is Serine (Ser) or Threonine (Thr); X2 is any amino acid except for Proline (Pro); and wherein N* is Asparagine (Asn) covalently attached in a β1 linkage to an N-glycan. The N-glycan may be a molecule having a structure selected from N-glycans in the group consisting of Man₍₁₋₉₎GlcNAc₂; or selected from N-glycans in the group consisting of GlcNAc₍₁₋₄₎Man₃GlcNAc₂; or selected from N-glycans in the group consisting of Gal₍₁₋₄₎GlcNAc₍₁₋₄₎Man₃GlcNAc₂; or selected from N-glycans in the group consisting of NANA₍₁₋₄₎Gal₍₁₋₄₎GlcNAc₍₁₋₄₎Man₃GlcNAc₂. The N-glycan may be selected from the group of N-glycan structures 1 to 106 shown herein. In particular embodiments, the N-glycan is a paucimannose (Man₃GlcNAc₂) or a Man₅GlcNAc₂.

(SEQ INO: 177)
FVN*QX1LCGSHLVEALYLVCGERGFFYTPKT
(SEQ ID NO: 253)
FVNQHLCGSHLVEALYLVCGERGFN*YTPKT
(SEQ ID NO: 254)
FVNQHLCGSHLVEALYLVCGERGFFYTN*KT
(SEQ INO: 178)
FVNQHLCGSHLVEALYLVCGERGFN*YTN*KT
(SEQ INO: 179)
FVN*QX1LCGSHLVEALYLVCGERGFN*YTPKT
(SEQ INO: 180)
FVN*QX1LCGSHLVEALYLVCGERGFFYTN*KT
(SEQ INO: 181)
FVN*QX1LCGSHLVEALYLVCGERGFN*YTN*KT
(SEQ INO: 182)
N*X2X1FVNQHLCGSHLVEALYLVCGERGFFYTPKT
(SEQ INO: 183)
N*X2X1FVN*QX1LCGSHLVEALYLVCGERGFFYTPKT
(SEQ INO: 184)
N*X2X1FVNQHLCGSHLVEALYLVCGERGFN*YTPKT
(SEQ INO: 185)
N*X2X1FVNQHLCGSHLVEALYLVCGERGFFYTN*KT
(SEQ INO: 186)
N*X2X1FVNQHLCGSHLVEALYLVCGERGFN*YTN*KT
(SEQ INO: 187)
N*X2X1FVN*QX1LCGSHLVEALYLVCGERGFN*YTPKT
(SEQ INO: 188)
N*X2X1FVN*QX1LCGSHLVEALYLVCGERGFFYTN*KT
(SEQ INO: 189)
N*X2X1FVN*QXLCGSHLVEALYLVCGERGFN*YTN*KT
(SEQ INO: 190)
FVNQHLCGSHLVEALYLVCGERGFFYTPKTN*
(SEQ INO: 191)
FVN*QX1LCGSHLVEALYLVCGERGFFYTPKTN*
(SEQ INO: 192)
FVNQHLCGSHLVEALYLVCGERGFN*YTPKTN*
(SEQ INO: 193)
FVNQHLCGSHLVEALYLVCGERGFFYTN*KTN*
(SEQ INO: 194)
FVNQHLCGSHLVEALYLVCGERGFN*YTN*KTN*
(SEQ INO: 195)
FVN*QX1LCGSHLVEALYLVCGERGFN*YTPKTN*
(SEQ INO: 196)
FVN*QX1LCGSHLVEALYLVCGERGFFYTN*KTN*
(SEQ INO: 197)
FVN*QX1LCGSHLVEALYLVCGERGFN*YTN*KTN*
(SEQ INO: 198)
N*X2X1FVNQHLCGSHLVEALYLVCGERGFFYTPKTN*
(SEQ INO: 199)
N*X2X1FVN*QX1LCGSHLVEALYLVCGERGFFYTPKTN*
(SEQ INO: 200)
N*X2X1FVNQHLCGSHLVEALYLVCGERGFN*YTPKTN*
(SEQ INO: 201)
N*X2X1FVNQHLCGSHLVEALYLVCGERGFFYTN*KTN*
(SEQ INO: 202)
N*X2X1FVNQHLCGSHLVEALYLVCGERGFN*YTN*KTN*
(SEQ INO: 203)
N*X2X1FVN*QX1LCGSHLVEALYLVCGERGFN*YTPKTN*
(SEQ INO: 204)
N*X2X1FVN*QX1LCGSHLVEALYLVCGERGFFYTN*KTN*
(SEQ INO: 205)
N*X2X1FVN*QX1LCGSHLVEALYLVCGERGFN*YTN*KTN*
(SEQ INO: 206)
FVN*QX1LCGSHLVEALYLVCGERGFFYTPKTRR
(SEQ INO: 207)
FVNQHLCGSHLVEALYLVCGERGFN*YTPKTRR
(SEQ INO: 208)
FVNQHLCGSHLVEALYLVCGERGFFYTN*KTRR
(SEQ INO: 209)
FVNQHLCGSHLVEALYLVCGERGFN*YTN*KTRR
(SEQ INO: 210)
FVN*QX1LCGSHLVEALYLVCGERGFN*YTPKTRR
(SEQ INO: 211)
FVN*QX1LCGSHLVEALYLVCGERGFFYTN*KTRR
(SEQ INO: 212)
FVN*QX1LCGSHLVEALYLVCGERGFN*YTN*KTRR
(SEQ INO: 213)
N*X2X1FVNQHLCGSHLVEALYLVCGERGFFYTPKTRR
(SEQ INO: 214)
N*X2X1FVN*QX1LCGSHLVEALYLVCGERGFFYTPKTRR
(SEQ INO: 215)
N*X2X1FVNQHLCGSHLVEALYLVCGERGFN*YTPKTRR
(SEQ INO: 216)
N*X2X1FVNQHLCGSHLVEALYLVCGERGFFYTN*KTRR
(SEQ INO: 217)
N*X2X1FVNQHLCGSHLVEALYLVCGERGFN*YTN*KTRR
(SEQ INO: 218)
N*X2X1FVN*QX1LCGSHLVEALYLVCGERGFN*YTPKTRR
(SEQ INO: 219)
N*X2X1FVN*QX1LCGSHLVEALYLVCGERGFFYTN*KTRR
(SEQ INO: 220)
N*X2X1FVN*QX1LCGSHLVEALYLVCGERGFN*YTN*KTRR
(SEQ INO: 221)
FVNQHLCGSHLVEALYLVCGERGFFYTPKTN*X2X1RR
(SEQ INO: 222)
FVN*QX1LCGSHLVEALYLVCGERGFFYTPKTN*X2X1RR
(SEQ INO: 223)
FVNQHLCGSHLVEALYLVCGERGFN*YTPKTN*X2X1RR
(SEQ INO: 224)
FVNQHLCGSHLVEALYLVCGERGFFYTN*KTN*X2X1RR
(SEQ INO: 225)
FVNQHLCGSHLVEALYLVCGERGFN*YTN*KTN*X2X1RR
(SEQ INO: 226)
FVN*QX1LCGSHLVEALYLVCGERGFN*YTPKTN*X2X1RR
(SEQ INO: 227)
FVNQ*X1LCGSHLVEALYLVCGERGFFYTN*KTN*X2X1RR
(SEQ INO: 228)
FVN*QX1LCGSHLVEALYLVCGERGFN*YTN*KTN*X2X1RR
(SEQ INO: 229)
N*X2X1FVNQHLCGSHLVEALYLVCGERGFFYTPKTN*X2X1RR
(SEQ INO: 230)
N*X2X1FVN*QX1LCGSHLVEALYLVCGERGFFYTPKTN*X2X1RR
(SEQ INO: 231)
N*X2X1FVNQHLCGSHLVEALYLVCGERGFN*YTPKTN*X2X1RR
(SEQ INO: 232)
N*X2X1FVNQHLCGSHLVEALYLVCGERGFFYTN*KTN*X2X1RR
(SEQ INO: 233)
N*X2X1FVNQHLCGSHLVEALYLVCGERGFN*YTN*KTN*X2X1RR
(SEQ INO: 234)
N*X2X1FVN*QX1LCGSHLVEALYLVCGERGFN*YTPKTN*X2X1RR
(SEQ INO: 235)
N*X2X1FVN*QX1LCGSHLVEALYLVCGERGFFYTN*KTN*X2X1RR
(SEQ INO: 236)
N*X2X1FVN*QX1LCGSHLVEALYLVCGERGFN*YTN*KTN*X2X1RR
(SEQ INO: 237)
FVN*QX1LCGSHLVEALYLVCGERGFFYTPK
(SEQ ID NO: 238)
FVNQHLCGSHLVEALYLVCGERGFN*YTPK
(SEQ ID NO: 239)
FVNQHLCGSHLVEALYLVCGERGFFYTN*K
(SEQ INO: 240)
FVNQHLCGSHLVEALYLVCGERGFN*YTN*K
(SEQ INO: 241)
FVN*QX1LCGSHLVEALYLVCGERGFN*YTPK
(SEQ INO: 242)
FVN*QX1LCGSHLVEALYLVCGERGFFYTN*K
(SEQ INO: 243)
FVN*QX1LCGSHLVEALYLVCGERGFN*YTN*K
(SEQ INO: 244)
N*X2X1FVNQHLCGSHLVEALYLVCGERGFFYTPK
(SEQ INO: 245)
N*X2X1FVN*QX1LCGSHLVEALYLVCGERGFFYTPK
(SEQ INO: 246)
N*X2X1FVNQHLCGSHLVEALYLVCGERGFN*YTPK
(SEQ INO: 247)
N*X2X1FVNQHLCGSHLVEALYLVCGERGFFYTN*K
(SEQ INO: 248)
N*X2X1FVNQHLCGSHLVEALYLVCGERGFN*YTN*K
(SEQ INO: 249)
N*X2X1FVN*QX1LCGSHLVEALYLVCGERGFN*YTPK
(SEQ INO: 250)
N*X2X1FVN*QX1LCGSHLVEALYLVCGERGFFYTN*K
(SEQ INO: 251)
N*X2X1FVN*QXLCGSHLVEALYLVCGERGFN*YTN*K
(SEQ INO: 319)
N*TTFVNQHLCGSHLVEALYLVCGERGFFYTPKTRR
(SEQ INO: 320)
N*TTFVNQHLCGSHLVEALYLVCGERGFFYTN*KTRR
(SEQ INO: 321)
FVN*ETLCGSHLVEALYLVCGERGFFYTPKTRR
(SEQ INO: 322)
FVNQHLCGSHLVEALYLVCGERGFN*YTPKTRR
(SEQ INO: 323)
FVNQHLCGSHLVEALYLVCGERGFN*FTPKTRR
(SEQ INO: 324)
FVN*QTLCGSHLVEALYLVCGERGFFYTN*KTRR
(SEQ INO: 325)
FVN*ETLCGSHLVEALYLVCGERGFFYTN*KTRR
(SEQ INO: 326)
FVNQHLCGSHLVEALYLVCGERGFN*YTN*KTRR
(SEQ INO: 327)
FVNQHLCGSHLVEALYLVCGERGFFYTN*KTRR
(SEQ INO: 328)
N*GTFVNQHLCGSHLVEALYLVCGERGFFYTDKT
(SEQ INO: 329)
N*GTFVNQHLCGSHLVEALYLVCGERGFFYTDK
(SEQ INO: 330)
N*GTFVN*ETLCGSHLVEALYLVCGERGFFYTDKT
(SEQ INO: 331)
N*GTFVN*ETLCGSHLVEALYLVCGERGFFYTDK
(SEQ INO: 332)
FVN*ETLCGSHLVEALYLVCGERGFN*FTDKT
(SEQ INO: 333)
FVN*ETLCGSHLVEALYLVCGERGFN*FTDK
(SEQ INO: 334)
N*GTFVNQHLCGSHLVEALYLVCGERGFFYTKPT
(SEQ INO: 335)
N*GTFVKQHLCGSHLVEALYLVCGERGFFYTPET
(SEQ INO: 336)
N*GTFVN*ETLCGSHLVEALYLVCGERGFFYTDKT
(SEQ INO: 337)
N*GTFVN*ETLCGSHLVEALYLVCGERGFN*YTDK

Wherein in the preceding B-chain sequences X1 is Serine (Ser) or Threonine (Thr); X2 is any amino acid except for Proline (Pro); and wherein N* is Asparagine (Asn) covalently attached in a β1 linkage to an N-glycan. The N-glycan may be a molecule having a structure selected from N-glycans in the group consisting of Man₍₁₋₉₎GlcNAc₂; or selected from N-glycans in the group consisting of GlcNAc₍₁₋₄₎Man₃GlcNAc₂; or selected from N-glycans in the group consisting of Gal₍₁₋₄₎GlcNAc₍₁₋₄₎Man₃GlcNAc₂; or selected from N-glycans in the group consisting of NANA₍₁₋₄₎Gal₍₁₋₄₎GlcNAc₍₁₋₄₎Man₃GlcNAc₂. The N-glycan may be selected from the group of N-glycan structures 1 to 106 shown herein. In particular embodiments, the N-glycan is a paucimannose (Man₃GlcNAc₂) or a Man₅GlcNAc₂.

In another aspect, the N-glycosylated insulin analogue is an N-glycosylated single-chain insulin analogue comprising the B-chain peptide and the A-chain peptide of human insulin or analogues or derivatives thereof, e.g., any one of the aforementioned derivatives including any combination of A- and B-chain peptides having an amino acid sequence selected from the group of sequences shown by SEQ ID NOs:162 to 254 and 316 to 337 or in combination with a native A- or B-chain provided that at least one asparagine residue in the single-chain insulin analogue is attached to an N-glycan, connected by a connecting peptide, wherein the connecting peptide may vary from 3 amino acid residues and up to a length corresponding to the length of the natural C-peptide in human insulin with the proviso that at least one of the B-chain peptide, A-chain peptide, or connecting peptide comprises an N-glycan attached thereto. The connecting peptide in the N-glycosylated single-chain insulin analogue is however normally shorter than the human C-peptide and will typically have a length from 3 to about 35, from 3 to about 30, from 4 to about 35, from 4 to about 30, from 5 to about 35, from 5 to about 30, from 6 to about 35 or from 6 to about 30, from 3 to about 25, from 3 to about 20, from 4 to about 25, from 4 to about 20, from 5 to about 25, from 5 to about 20, from 6 to about 25 or from 6 to about 20, from 3 to about 15, from 3 to about 10, from 4 to about 15, from 4 to about 10, from 5 to about 15, from 5 to about 10, from 6 to about 15 or from 6 to about 10, or from 6-9, 6-8, 6-7, 7-8, 7-9, or 7-10 amino acid residues in the peptide chain. Single-chain peptides have been disclosed in U.S. Published Application No. 20080057004, U.S. Pat. No. 6,630,348, International Application Nos. WO2005054291, WO2007104734, WO2010080609, WO20100099601, and WO2011159895, each of which is incorporated herein by reference. Further provided are compositions and formulations of the above comprising a pharmaceutically acceptable carrier, salt, or combination thereof.

In particular embodiments the N-glycosylated single-chain insulin analogue connecting peptide comprises the formula Gly-Z¹-Gly-Z² wherein Z¹ is Asn or another amino acid except for tyrosine, and Z² is a peptide of 2-35 amino acids. In particular embodiments, the connecting peptide comprises at least one attachment site comprising the sequence Asn-Xaa-Ser/Thr wherein Xaa is any amino acid except proline. For example, when Z¹ is Asn, then the N-terminal amino acid of Z² is Ser or Thr.

In particular embodiments, the N-glycosylated single-chain insulin analogue connecting peptide is GNGSSSRRAPQT (SEQ INO:258), GAGNSSRRAPQT (SEQ INO:259), GAGSNSSRRAPQT (SEQ INO:260), GNGSNSSRRAPQT (SEQ INO:261), GAGSSSRRANQT (SEQ INO:262), GNGSSSRRANQT (SEQ INO:263), GAGNSSRRANQT (SEQ INO:264), GAGSNSSRRANQT (SEQ INO:265), GNGSNSSRRANQT (SEQ INO:266), GAGSSSRRAPQT (SEQ INO:267), GGGPRR (SEQ INO:268), GGGPGAG (SEQ INO:269), GGGGGKR (SEQ INO:270), or GGGPGKR (SEQ INO:271).

In particular embodiments, the N-glycosylated single-chain insulin analogue connecting peptide is VGLSSGQ (SEQ INO:272) or TGLGSGR (SEQ INO:273). In other aspects, the N-glycosylated single-chain insulin analogue connecting peptide is RRGPGGG (SEQ INO:274), RRGGGGG (SEQ INO:275), GGAPGDVKR (SEQ INO:276), RRAPGDVGG (SEQ INO:277), GGYPGDVLR (SEQ INO:278), RRYPGDVGG (SEQ INO:279), GGHPGDVR (SEQ INO:280), or RRAPGDVGG (SEQ INO:281).

In particular embodiments, the single-chain N-glycosylated insulin analogue comprises (1) any combination of A- and B-chain peptides having an amino acid sequence selected from the group of sequences shown by SEQ ID NOs:162 to 254 and 316 to 337 or in combination with a native A- or B-chain and (2) any aforementioned connecting peptide, provided that at least one asparagine residue in the single-chain insulin analogue is attached to an N-glycan. In particular embodiments, the B chain may lack one, two, three, four, or five amino acids at the C-terminus. In a further embodiment, the B-chain is desB30 or desB26-30. The N-glycan may be a molecule having a structure selected from N-glycans in the group consisting of Man₍₁₋₉₎GlcNAc₂; or selected from N-glycans in the group consisting of GlcNAc₍₁₋₄₎Man₃GlcNAc₂; or selected from N-glycans in the group consisting of Gal₍₁₋₄₎GlcNAc₍₁₋₄₎Man₃GlcNAc₂; or selected from N-glycans in the group consisting of NANA₍₁₋₄₎Gal₍₁₋₄₎GlcNAc₍₁₋₄₎Man₃GlcNAc₂. The N-glycan may be selected from the group of N-glycan structures 1 to 106 shown herein. In particular embodiments, the N-glycan is a paucimannose (Man₃GlcNAc₂) or a Man₅GlcNAc₂. Further provided are compositions and formulations of the above comprising a pharmaceutically acceptable carrier, salt, or combination thereof.

In particular embodiments, the single-chain N-glycosylated insulin analogue comprises (1) any combination of A- and B-chain peptides having an amino acid sequence selected from the group of sequences shown by SEQ ID NOs:162 to 254 and 316 to 337 or in combination with a native A- or B-chain and (2) a connecting peptide having an amino acid sequence shown by SEQ ID NOs:258-281, provided that at least one asparagine residue in the single-chain insulin analogue is attached to an N-glycan. Further provided are compositions and formulations of the above comprising a pharmaceutically acceptable carrier, salt, or combination thereof.

In particular embodiments, the N-glycosylated single-chain insulin analogue connecting peptide is GN*GSSSRRAPQT (SEQ INO:283), GAGN*SSRRAPQT (SEQ INO:284), GAGSN*SSRRAPQT (SEQ INO:285), GN*GSN*SSRRAPQT (SEQ INO:286), GAGSSSRRAN*QT (SEQ INO:287), GN*GSSSRRAN*QT (SEQ INO:288), GAGN*SSRRAN*QT (SEQ INO:289), GAGSN*SSRRAN*QT (SEQ INO:290), or GN*GSN*SSRRAN*QT (SEQ INO:291), wherein N* is Asparagine (Asn) covalently attached in a β1 linkage to an N-glycan. The N-glycan may be a molecule having a structure selected from N-glycans in the group consisting of Man₍₁₋₉₎GlcNAc₂; or selected from N-glycans in the group consisting of GlcNAc₍₁₋₄₎Man₃GlcNAc₂; or selected from N-glycans in the group consisting of Gal₍₁₋₄₎GlcNAc₍₁₋₄₎Man₃GlcNAc₂; or selected from N-glycans in the group consisting of NANA₍₁₋₄₎Gal₍₁₋₄₎GlcNAc₍₁₋₄₎Man₃GlcNAc₂. The N-glycan may be selected from the group of N-glycan structures 1 to 106 shown herein. In particular embodiments, the N-glycan is a paucimannose (Man₃GlcNAc₂) or a Man₅GlcNAc₂.

In particular embodiments, the single-chain N-glycosylated insulin analogue comprises (1) a native A-chain and B-chain and (2) an N-glycosylated connecting peptide having an amino acid sequence shown by SEQ ID NOs:282-290. The N-glycan of the single-chain N-glycosylated insulin analogue may be a molecule having a structure selected from N-glycans in the group consisting of Man₍₁₋₉₎GlcNAc₂; or selected from N-glycans in the group consisting of GlcNAc₍₁₋₄₎Man₃GlcNAc₂; or selected from N-glycans in the group consisting of Gal₍₁₋₄₎GlcNAc₍₁₋₄₎Man₃GlcNAc₂; or selected from N-glycans in the group consisting of NANA₍₁₋₄₎Gal₍₁₋₄₎GlcNAc₍₁₋₄₎Man₃GlcNAc₂. The N-glycan may be selected from the group of N-glycan structures 1 to 106 shown herein. In particular embodiments, the N-glycan is a paucimannose (Man₃GlcNAc₂) or a Man₅GlcNAc₂. Further provided are compositions and formulations of the above comprising a pharmaceutically acceptable carrier, salt, or combination thereof.

In particular embodiments, the single-chain N-glycosylated insulin analogue comprises (1) a native A-chain and B-chain or analogue thereof having 1, 2, 3, 4, 5, or more amino acid substitutions and/or deletions and (2) any aforementioned connecting peptide provided that at least one NH₂, COOH, SH, or imidizole ring of His is directly or indirectly conjugated to an N-glycan. The N-glycan of the single-chain N-glycosylated insulin analogue may be a molecule having a structure selected from N-glycans in the group consisting of Man₍₁₋₉₎GlcNAc₂; or selected from N-glycans in the group consisting of GlcNAc₍₁₋₄₎Man₃GlcNAc₂; or selected from N-glycans in the group consisting of Gal₍₁₋₄₎GlcNAc₍₁₋₄₎Man₃GlcNAc₂; or selected from N-glycans in the group consisting of NANA₍₁₋₄₎Gal₍₁₋₄₎GlcNAc₍₁₋₄₎Man₃GlcNAc₂. The N-glycan may be selected from the group of N-glycan structures 1 to 106 shown herein. In particular embodiments, the N-glycan is a paucimannose (Man₃GlcNAc₂) or a Man₅GlcNAc₂. Further provided are compositions and formulations of the above comprising a pharmaceutically acceptable carrier, salt, or combination thereof.

In particular embodiments, the N-glycan is directly or indirectly conjugated to an attachment site in vitro by way of optional linker or spacer as disclosed above. In further embodiments, the optional linker or spacer comprises a chain of atoms from 1 to about 60, or 1 to 30 atoms or longer, 2 to 5 atoms, 2 to 10 atoms, 5 to 10 atoms, or 10 to 20 atoms long. In some embodiments, the chain atoms are all carbon atoms. In some embodiments, the chain atoms in the backbone of the linker or spacer are selected from the group consisting of C, O, N, and S. Chain atoms and linkers or spacers may be selected according to their expected solubility (hydrophilicity) so as to provide a more soluble conjugate. In some embodiments, the linker or spacer provides a functional group that is subject to cleavage by an enzyme or other catalyst or hydrolytic conditions found in the target tissue or organ or cell. In some embodiments, the length of the linker or spacer is long enough to reduce the potential for steric hindrance. If the linker or spacer is a covalent bond or a peptidyl bond and the insulin analogue is conjugated to a heterologous polypeptide, e.g., immunoglobulin, Fc fragment of an immunoglobulin, human serum albumin, the entire conjugate can be a fusion protein. Such peptidyl linkers may be any length. Exemplary linkers are from about 1 to 50 amino acids in length, 5 to 50, 3 to 5, 5 to 10, 5 to 15, or 10 to 30 amino acids in length. Further provided are compositions and formulations of the above comprising a pharmaceutically acceptable carrier, salt, or combination thereof.

In particular embodiments, the linker or spacer may be (i) one, two, three, or more unbranched alkane α, ω-dicarboxylic acid groups having one to seven methylene groups; (ii) one, two, three, or more amino acids; or, (iii) one, two, three, or more γ-aminobutanyl residues. In particular embodiments, the optional linker or spacer may be one, two, three, or more γ-glutamyl residues; one, two, three, or more β-alanyl residues; one, two, three, or more β-asparagyl residues; or one, two, three, or more glycyl residues.

In particular embodiments, the linker or spacer may be a covalent bond; a carbon atom; a heteroatom, an optionally substituted group selected from the group consisting of acyl, aliphatic, heteroaliphatic, aryl, heteroaryl, and heterocyclic; a bivalent, straight or branched, saturated or unsaturated, optionally substituted C1-30 hydrocarbon chain wherein one or more methylene units are optionally and independently replaced by —O—, —S—, —N(R)—, —C(O)—, C(O)O—, OC(O)—, —N(R)C(O)—, —C(O)N(R)—, —S(O)—, —S(O)2-, —N(R)SO2-, SO2N(R)—;

each occurrence of R is independently hydrogen, a suitable protecting group, or an acyl moiety, arylalkyl moiety, aliphatic moiety, aryl moiety, heteroaryl moiety, or heteroaliphatic moiety.

### III. Insulin Analogues

In various embodiments of the in vivo N-glycosylated insulin or insulin analogues disclosed herein, the glycosylation is N-linked and the attachment group is at B28 (P is replaced with N). However, in embodiments in which the N-linked glycosylated insulin analogue includes a mutation at position B28 to an amino acid residue other than asparagine, then the N-linked glycosylation site (attachment group) is selected to be in another position in the molecule, for example selected to be at B-2, B3, B25, A-2, A8, A10, or A21. For example, insulin lispro (HUMALOG) is a rapid acting insulin analogue in which the penultimate lysine and proline residues on the C-terminal end of the B-peptide have been reversed (LysB28ProB29-human insulin), which reduces the formation of insulin multimers. Insulin aspart (NOVOLOG) is another rapid acting insulin mutant in which the proline at position B28 has been substituted with aspartic acid (AspB28-human insulin). This mutation also results in reduced formation of multimers. Therefore, those glycosylated insulins disclosed herein in which the attachment group is at position 28 (i.e., the proline at position B28 is replaced with asparagine to make an N-linked glycosylation site or in which an oligosaccharide or glycan is chemically conjugated to the amino acid at B28 or B29 (e.g., conjugated to the lysine at position 29 or lysine at position 28) will have reduced ability to form multimers and thus, may exhibit a fast-acting profile. In some embodiments, the mutation at positions B28 and/or B29 is accompanied by one or more mutations elsewhere in the insulin polypeptide. For example, insulin glulisine (APIDRA) is yet another rapid acting insulin mutant in which asparagine at position B3 has been replaced by a lysine residue and lysine at position B29 has been replaced with a glutamic acid residue (LysB3GluB29-human insulin). This analogue may be conjugated to an oligosaccharide or glycan at the lysine residue at B3.

In various embodiments, the in vitro glycosylated or in vivo N-glycosylated insulin analogue has an isoelectric point that has been shifted relative to human insulin. In some embodiments, the shift in isoelectric point is achieved by adding one or more arginine, lysine, or histidine residues to the N-terminus of the insulin A-chain peptide and/or the C-terminus of the insulin B-chain peptide. Examples of such insulin polypeptides include ArgA0-human insulin, ArgB31ArgB32-human insulin, GlyA21ArgB31ArgB32-human insulin, ArgA0ArgB31ArgB32-human insulin, and ArgA0GlyA21ArgB31ArgB32-human insulin. By way of further example, insulin glargine (LANTUS) is an exemplary long-acting insulin analogue in which AsnA21 has been replaced by glycine, and two arginine residues have been covalently linked to the C-terminus of the B-peptide. The effect of these amino acid changes was to shift the isoelectric point of the molecule, thereby producing a molecule that is soluble at acidic pH (e.g., pH 4 to 6.5) but insoluble at physiological pH. When a solution of insulin glargine is injected into the muscle, the pH of the solution is neutralized and the insulin glargine forms microprecipitates that slowly release the insulin glargine over the 24 hour period following injection with no pronounced insulin peak and thus a reduced risk of inducing hypoglycemia. This profile allows a once-daily dosing to provide a patient's basal insulin. Thus, in some embodiments, the insulin analogue comprises an A-chain peptide wherein the amino acid at position A21 is glycine and a B-chain peptide wherein the amino acids at position B31 and B32 are arginine. The present disclosure encompasses all single and multiple combinations of these mutations and any other mutations that are described herein (e.g., GlyA21-human insulin, GlyA21ArgB31-human insulin, ArgB31ArgB32-human insulin, ArgB31-human insulin).

In various embodiments, the in vitro glycosylated or in vivo N-glycosylated insulin analogue is truncated. For example, in certain embodiments, the B-chain peptide lacks at least one B1, B2, B3, B26, B27, B28, B29, or B30. In particular embodiments, the B-chain peptide lacks a combination of residues. For example, the B-chain may be truncated to lack amino acid residues B1-B2, B1-B3, B1-B4, B29-B30, B28-B30, B27-B30 and/or B26-B30. In some embodiments, these deletions and/or truncations apply to any of the aforementioned insulin analogues (e.g., without limitation to produce des(B29)-insulin lispro, des(B30)-insulin aspart, and the like.

In some embodiments, the in vitro glycosylated or in vivo N-glycosylated insulin analogue contains additional amino acid residues on the N- or C-terminus of the A-chain peptide or B-peptide. In some embodiments, one or more amino acid residues are located at positions A0, A22, B0 and/or B31. In some embodiments, one or more amino acid residues are located at position A0. In some embodiments, one or more amino acid residues are located at position A22. In some embodiments, one or more amino acid residues are located at position B0. In some embodiments, one or more amino acid residues are located at position B31. In particular embodiments, the glycosylated insulin or insulin analogue does not include any additional amino acid residues at positions A0, A22, B0 or B31.

In particular embodiments, one or more amidated amino acids of the in vitro glycosylated or in vivo N-glycosylated insulin analogue are replaced with an acidic amino acid, or another amino acid. For example, the asparagine at positions other than the position glycosylated may be replaced with aspartic acid or glutamic acid, or another residue. Likewise, glutamine may be replaced with aspartic acid or glutamic acid, or another residue. In particular, AsnA18, AsnA21, or AsnB3, or any combination of those residues, may be replaced by aspartic acid or glutamic acid, or another residue. GlnA15 or GlnB4, or both, may be replaced by aspartic acid or glutamic acid, or another residue. In particular embodiments, the insulin analogues have an aspartic acid, or another residue, at position A21 or aspartic acid, or another residue, at position B3, or both.

One skilled in the art will recognize that it is possible to replace yet other amino acids in the in vitro glycosylated or in vivo N-glycosylated insulin analogue with other amino acids while retaining biological activity of the molecule. For example, without limitation, the following modifications are also widely accepted in the art: replacement of the histidine residue of position B10 with aspartic acid (HisB10 to AspB10); replacement of the phenylalanine residue at position B1 with aspartic acid (PheB1 to AspB1); replacement of the threonine residue at position B30 with alanine (ThrB30 to AlaB30); replacement of the tyrosine residue at position B26 with alanine (TyrB26 to AlaB26); and replacement of the serine residue at position B9 with aspartic acid (SerB9 to AspB9).

In various embodiments, the in vitro glycosylated or in vivo N-glycosylated insulin analogue has a protracted profile of action. Thus, in certain embodiments, the in vitro glycosylated or in vivo N-glycosylated insulin analogue may be acylated with a fatty acid. That is, an amide bond is formed between an amino group on the insulin analogue and the carboxylic acid group of the fatty acid. The amino group may be the alpha-amino group of an N-terminal amino acid of the insulin analogue, or may be the epsilon-amino group of a lysine residue of the insulin analogue. The in vitro glycosylated or in vivo N-glycosylated insulin analogue may be acylated at one or more of the three amino groups that are present in wild-type human insulin may be acylated on lysine residue that has been introduced into the wild-type human insulin sequence. In particular embodiments, the in vitro glycosylated or in vivo N-glycosylated insulin analogue may be acylated at position B1. In certain embodiments, the in vitro glycosylated or in vivo N-glycosylated insulin analogue may be acylated at position B29. In certain embodiments, the fatty acid is selected from myristic acid (C₁₄), pentadecylic acid (C₁₅), palmitic acid (C₁₆), heptadecylic acid (C₁₇) and stearic acid (C₁₈). For example, insulin detemir (LEVEMIR) is a long acting insulin mutant in which ThrB30 has been deleted (desB30) and a C₁₄ fatty acid chain (myristic acid) has been attached to LysB29 via a γE linker and insulin degludec is a long acting insulin mutant in which ThrB30 has been deleted and a C₁₆ fatty acid chain (palmitic acid) has been attached to LysB29 via a γE linker.

The in vitro glycosylated or in vivo N-glycosylated insulin analogue molecule comprising one or more N-linked glycosylation sites, includes heterodimer analogues and single-chain analogues that comprise modified derivatives of the native A-chain and/or B-chain, including modification of the amino acid at position A19, B16 or B25 to a 4-amino phenylalanine or one or more amino acid substitutions at positions selected from A5, A8, A9, A10, A12, A13, A14, A15, A17, A18, A21, B1, B2, B3, B4, B5, B9, B10, B13, B14, B16, B17, B18, B20, B21, B22, B23, B26, B27, B28, B29 and B30 or deletions of any or all of positions B1-4 and B26-30. Examples of insulin analogues can be found for example in published International Application WO9634882, WO95516708; WO20100080606, WO2009/099763, and WO2010080609, U.S. Pat. No. 6,630,348, and Kristensen et al., Biochem. J. 305: 981-986 (1995), the disclosures of which are incorporated herein by reference). In further embodiments, the in vitro glycosylated or in vivo N-glycosylated insulin analogues may be acylated and/or pegylated.

In some embodiments, the N-terminus of the A-peptide, the N-terminus of the B-peptide, the epsilon-amino group of Lys at position B29 or any other available amino group in the in vitro glycosylated or in vivo N-glycosylated insulin analogue is covalently linked to a fatty acid moiety of general formula:

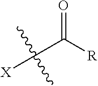

wherein X is an amino group of the insulin polypeptide and R is H or a C₁₋₃₀ alkyl group and the insulin analogue comprises one or more N-linked glycosylation sites. In some embodiments, R is a C₁₋₂₀ alkyl group, a C₃₋₁₉ alkyl group, a C₅₋₁₈ alkyl group, a C₆₋₁₇ alkyl group, a C₈₋₁₆ alkyl group, a C₁₀₋₁₅ alkyl group, or a C₁₂₋₁₄ alkyl group. In certain embodiments, the insulin polypeptide is conjugated to the moiety at the A1 position. In particular embodiments, the insulin polypeptide is conjugated to the moiety at the B1 position. In particular embodiments, the insulin polypeptide is conjugated to the moiety at the epsilon-amino group of Lys at position B29. In particular embodiments, position B28 of the in vitro glycosylated or in vivo N-glycosylated insulin analogue is Lys and the epsilon-amino group of Lys^{B28} is conjugated to the fatty acid moiety. In particular embodiments, position B3 of the in vitro glycosylated or in vivo N-glycosylated insulin analogue is Lys and the epsilon-amino group of Lys^{B3} is conjugated to the fatty acid moiety. In some embodiments, the fatty acid chain is 8-20 carbons long. In particular embodiments, the fatty acid is octanoic acid (C8), nonanoic acid (C9), decanoic acid (C10), undecanoic acid (C11), dodecanoic acid (C12), or tridecanoic acid (C13). In certain embodiments, the fatty acid is myristic acid (C14), pentadecanoic acid (C15), palmitic acid (C16), heptadecanoic acid (C17), stearic acid (C18), nonadecanoic acid (C19), or arachidic acid (C20). In particular embodiments, the glycosylated insulin analogue comprises at least one N-glycan as disclosed herein attached to the asparagine residue comprising an N-linked glycosylation site or an asparagine residue which had comprised an N-linked glycosylation site when the asparagine residue is at position B28 and glycosylated insulin analogue is desB30.

In particular embodiments, the in vitro glycosylated or in vivo N-glycosylated insulin analogue comprises the mutations and/or chemical modifications of one of the following insulin analogues: Lys^{B28}Pro^{B29}-human insulin (insulin lispro), Asp^{B28}-human insulin (insulin aspart), Lys^{B3}Glu^{B29}-human insulin (insulin glulisine), Arg^{B31}Arg^{B32}-human insulin (insulin glargine), N^{εβ29}-myristoyl-des(B30)-human insulin (insulin detemir), Ala^{B26}-human insulin, Asp^{B1}-human insulin, Arg^{A0}-human insulin, Asp^{B1}Glu^{B13}-human insulin, Gly^{A21}-human insulin, Gly^{A21}Arg^{B31}Arg^{B32}-human insulin, Arg^{A0}Arg^{B31}Arg^{B32}-human insulin, Arg^{A0}Gly^{A21}Arg^{B31}Arg^{B32}-human insulin, des(B30)-human insulin, des(B27)-human insulin, des(B28-B30)-human insulin, des(B1)-human insulin, des(B1-B3)-human insulin. In particular embodiments, the glycosylated insulin analogue comprises at least one N-glycan as disclosed herein attached to the asparagine residue comprising an N-linked glycosylation site or an asparagine residue which had comprised an N-linked glycosylation site when the asparagine residue is at position B28 and glycosylated insulin analogue is desB30.

In particular embodiments, an in vitro glycosylated or in vivo N-glycosylated insulin analogue comprises the mutations and/or chemical modifications of one of the following insulin analogues: N^{εB29}-palmitoyl-human insulin, N^{εB29}-myrisotyl-human insulin, N^{εB28}-palmitoyl-Lys^{B28}Pro^{B29}-human insulin, N^{εB28}-myristoyl-Lys^{B28}Pro^{B29}-human insulin. In particular embodiments, the glycosylated insulin analogue comprises at least one N-glycan as disclosed herein attached to the asparagine residue comprising an N-linked glycosylation site.

In particular embodiments, the in vitro glycosylated or in vivo N-glycosylated insulin analogue comprises the mutations and/or chemical modifications of one of the following insulin analogues: N^{εB29}-palmitoyl-des(B30)-human insulin, N^{εB30}-myristoyl-Thr^{B29}Lys^{B30}-human insulin, N^{εB30}-palmitoyl-Thr^{B29}Lys^{B30}-human insulin, N^{εB29}-(N-palmitoyl-γ-glutamyl)-des(B30)-human insulin, N^{εB29}-(N-lithocolyl-γ-glutamyl)-des(B30)-human insulin, N^{εB29}-(ω-carboxyheptadecanoyl)-des(B30)-human insulin, N^{εB29}-(ω-carboxyheptadecanoyl)-human insulin. In particular embodiments, the glycosylated insulin analogue comprises at least one N-glycan as disclosed herein attached to the asparagine residue comprising an N-linked glycosylation site or an asparagine residue which had comprised an N-linked glycosylation site when the asparagine residue is at position B28 and glycosylated insulin analogue is desB30. In particular embodiments, the in vitro glycosylated or in vivo N-glycosylated insulin analogue comprises the mutations and/or chemical modifications of one of the following insulin analogues: N^{εB29}-octanoyl-human insulin, N^{εB29}-myristoyl-Gly^{A21}Arg^{B31}Arg^{B31}-human insulin, N^{εB29}-myristoyl-Gly^{A21}Gln^{B3}Arg^{B31}Arg^{B32}-human insulin, N^{εB29}-myristoyl-Arg^{A0}Gly^{A21}Arg^{B31}Arg^{B32}-human insulin, N^{εB29}-Arg^{A0}Gly^{A21}Gln^{B3}Arg^{B31}Arg^{B32}-human insulin, N^{εB29}-myristoyl-Arg^{A0}Gly^{A21}Arg^{B31}Arg^{B32}-human insulin, N^{εB29}-myristoyl-Arg^{B31}Arg^{B32}-human insulin, N^{εB29}-myristoyl-Arg^{A0}Gly^{A21}Arg^{B31}Arg^{B32}-human insulin, N^{εB29}-octanoyl-Gly^{A21}Arg^{B31}Arg^{B32}-human insulin, N^{εB29}-octanoyl-Gly^{A21}Gln^{B3}Arg^{B31}Arg^{B32}-human insulin, N^{εB29}-octanoyl-Arg^{A0}Gly^{A21}Arg^{B31}Arg^{B32}-human insulin, N^{εB29}-octanoyl-Arg^{A0}Gly^{A21}Gln^{B3}Arg^{B31}Arg^{B32}-human insulin, N^{εB29}-octanoyl-Arg^{B0}Gly^{A21}Asp^{B3}Arg^{B31}Arg^{B32}-human insulin, N^{εB29}-octanoyl-Arg^{B31}Arg^{B32}-human insulin, N^{εB29}-octanoyl-Arg^{A0}Arg^{B31}Arg^{B32}-human insulin. In particular embodiments, the glycosylated insulin analogue comprises at least one N-glycan as disclosed herein attached to the asparagine residue comprising an N-linked glycosylation site.

In particular embodiments, the in vitro glycosylated or in vivo N-glycosylated insulin analogue comprises the mutations and/or chemical modifications of one of the following insulin polypeptides: N^{εB28}-myristoyl-Gly^{A21}Lys^{B28}Pro^{B29}Arg^{B31}Arg^{B32}-human insulin, N^{εB28}-myristoyl-Gly^{A21}Gln^{B3}Lys^{B28}Pro^{B30}Arg^{B31}Arg^{B32}-human insulin, N^{εB28}-myristoyl-Arg^{A0}Gly^{A21}Lys^{B28}Pro^{B29}Arg^{B31}Arg^{B32}-human insulin, N^{εB28}-myristoyl-Arg^{A0}Gly^{A21}Gln^{B3}Lys^{B28}Pro^{B29}Arg^{B31}Arg^{B32}-human insulin, N^{εB28}-myristoyl-Arg^{A0}Gly^{A21}Asp^{B3}Lys^{B28}Pro^{B29}Arg^{B31}Arg^{B32}-human insulin, N^{εB28}-myristoyl-Lys^{B28}Pro^{B29}Arg^{B31}Arg^{B32}-human insulin, N^{εB28}-myristoyl-arg^{A0}Lys^{B28}Pro^{B29}Arg^{B31}Arg^{B32}-human insulin, N^{εB28}-octanoyl-Gly^{A21} Lys^{B28}Pro^{B29}Arg^{B31}Arg^{B32}-human insulin. In particular embodiments, the glycosylated insulin analogue comprises at least one N-glycan as disclosed herein attached to the asparagine residue comprising an N-linked glycosylation site.

In particular embodiments, the in vitro glycosylated or in vivo N-glycosylated insulin analogue comprises the mutations and/or chemical modifications of one of the following insulin analogues: N^{εB28}-octanoyl-Gly^{A21}Gln^{B3}Lys^{B28}Pro^{B29}Arg^{B31}Arg^{B32}-human insulin, N^{εB28}-octanoyl-Arg^{A0}Gly^{A21}Lys^{B28}Pro^{B29}Arg^{B31}Arg^{B32}-human insulin, N^{εB28}-octanoyl-Arg^{A0}Gly^{A21}Gln^{B3}Lys^{B28}Pro^{B29}Arg^{B31}Arg^{B32}-human insulin, N^{εB28}-octanoyl-Lys^{B28}Pro^{B29}Arg^{B31}Arg^{B32}-human insulin, N^{εB28}-octanoyl-Arg^{A0}Lys^{B28}Pro^{B29}Arg^{B31}Arg^{B32}-human insulin. In particular embodiments, the glycosylated insulin analogue comprises at least one N-glycan as disclosed herein attached to the asparagine residue comprising an N-linked glycosylation site.

In particular embodiments, the in vitro glycosylated or in vivo N-glycosylated insulin analogue comprises the mutations and/or chemical modifications of one of the following insulin analogues: N^{εB29}-tridecanoyl-des(B30)-human insulin, N^{εB29}-tetradecanoyl-des(B30)-human insulin N^{εB29}-decanoyl-des(B30)-human insulin, N^{εB29}-dodecanoyl-des(B30)-human insulin, N^{εB29}-tridecanoyl-Gly^{A21}-des(B30)-human insulin, N^{εB29}-tetradecanoyl-Gly^{A21}-des(B30)-human insulin, N^{εB29}-decanoyl-Gly^{A21}-des(B30)-human insulin, N^{εB29}-dodecanoyl-Gly^{A21}-des(B30)-human insulin, N^{εB29}-tridecanoyl-Gly^{A21}Gln^{B3}-des(B30)-human insulin, N^{εB29}-tetradecanoyl-Gly^{A21}Gln^{B3}-des(B30)-human insulin, N^{εB29}-decanoyl-Gly^{A21}Gln^{B3}-des(B30)-human insulin, N^{εB29}-dodecanoyl-Gly^{A21}-Gln^{B3}-des(B30)-human insulin, N^{εB29}-tridecanoyl-Ala^{A21}-des(B30)-human insulin, N^{εB29}-tetradecanoyl-Ala^{A21}-des(B30)-human insulin, N^{εB29}-decanoyl-Ala^{A21}-des(B30)-human insulin, N^{εB29}-dodecanoyl-Ala^{A21}-des(B30)-human insulin, N^{εB29}-tridecanoyl-Ala^{A21}-Gln^{B3}-des(B30)-human insulin, N^{εB29}-tetradecanoyl-Ala^{A21}Gln^{B3}-des(B30)-human insulin, N^{εB29}-decanoyl-Ala^{A21}Gln^{B3}-des(B30)-human insulin, N^{εB29}-dodecanoyl-Ala^{A21}Gln^{B3}-des(B30)-human insulin, N^{εB29}-tridecanoyl-Gln^{B3}-des(B30)-human insulin, N^{εB29}-tetradecanoyl-Gln^{B3}-des(B30)-human insulin, N^{εB29}-decanoyl-Gln^{B3}-des(B30)-human insulin, N^{εB29}-dodecanoyl-Gln^{B3}-des(B30)-human insulin. In particular embodiments, the glycosylated insulin analogue comprises at least one N-glycan as disclosed herein attached to the asparagine residue comprising an N-linked glycosylation site or an asparagine residue which had comprised an N-linked glycosylation site when the asparagine residue is at position B28 and glycosylated insulin analogue is desB30.

In particular embodiments, the in vitro glycosylated or in vivo N-glycosylated insulin analogue comprises the mutations and/or chemical modifications of one of the following insulin analogues: N^{εB29}-tridecanoyl-Gly^{A21}-human insulin, N^{εB29}-tetradecanoyl-Gly^{A21}-human insulin, N^{εB29}-decanoyl-Gly^{A21}-human insulin, N^{εB29}-dodecanoyl-Gly^{A21}-human insulin, N^{εB29}-tridecanoyl-Ala^{A21}-human insulin, N^{εB29}-tetradecanoyl-Ala^{A21}-human insulin, N^{εB29}-decanoyl-Ala^{A21}-human insulin, N^{εB29}-dodecanoyl-Ala^{A21}-human insulin. In particular embodiments, the glycosylated insulin analogue comprises at least one N-glycan as disclosed herein attached to the asparagine residue comprising an N-linked glycosylation site.

In particular embodiments, the in vitro glycosylated or in vivo N-glycosylated insulin analogue comprises the mutations and/or chemical modifications of one of the following insulin analogues: N^{εB29}-tridecanoyl-Gly^{A21}Gln^{B3}-human insulin, N^{εB29}-tetradecanoyl-Gly^{A21}Gln^{B3}-human insulin, N^{εB29}-decanoyl-Gly^{A21}Gln^{B3}-human insulin, N^{εB29}-dodecanoyl-Gly^{A21}Gln^{B3}-human insulin, N^{εB29}-tridecanoyl-Ala^{A21}Gln^{B3}-human insulin, N^{εB29}-tetradecanoyl-Ala^{A21}Gln^{B3}-human insulin, N^{εB29}-decanoyl-Ala^{A21}Gln^{B3}-human insulin, N^{εB29}-dodecanoyl-Ala^{A21}Gln^{B3}-human insulin. In particular embodiments, the glycosylated insulin analogue comprises at least one N-glycan as disclosed herein attached to the asparagine residue comprising an N-linked glycosylation site.

In particular embodiments, the in vitro glycosylated or in vivo N-glycosylated insulin analogue comprises the mutations and/or chemical modifications of one of the following insulin analogues: N^{εB29}-tridecanoyl-Gln^{B3}-human insulin, N^{εB29}-tetradecanoyl-Gln^{B3}-human insulin, N^{εB29}-decanoyl-Gln^{B3}-human insulin, N^{εB29}-dodecanoyl-Gln^{B3}-human insulin. In particular embodiments, the glycosylated insulin analogue comprises at least one N-glycan as disclosed herein attached to the asparagine residue comprising an N-linked glycosylation site.

In particular embodiments, the in vitro glycosylated or in vivo N-glycosylated insulin analogue comprises the mutations and/or chemical modifications of one of the following insulin analogues: N^{εB29}-tridecanoyl-Glu^{B30}-human insulin, N^{εB29}-tetradecanoyl-Glu^{B30}-human insulin, N^{εB29}-decanoyl-Glu^{B30}-human insulin, N^{εB29}-dodecanoyl-Glu^{B30}-human insulin. In particular embodiments, the glycosylated insulin analogue further includes at least one N-glycan as disclosed herein attached to the asparagine residue comprising an N-linked glycosylation site.

In particular embodiments, the in vitro glycosylated or in vivo N-glycosylated insulin analogue comprises the mutations and/or chemical modifications of one of the following insulin analogues: N^{εB29}-tridecanoyl-Gly^{A21}Gln^{B30}-human insulin, N^{εB29}-tetradecanoyl-Gly^{A21}Glu^{B30}-human insulin, N^{εB29}-decanoyl-Gly^{A21}Gln^{B30}-human insulin, N^{εB29}-dodecanoyl-Gly^{A21}Glu^{B30}-human insulin. In particular embodiments, the glycosylated insulin analogue further includes at least one N-glycan as disclosed herein attached to the asparagine residue comprising an N-linked glycosylation site.

In particular embodiments, the in vitro glycosylated or in vivo N-glycosylated insulin analogue comprises the mutations and/or chemical modifications of one of the following insulin analogues: N^{εB29}-tridecanoyl-Gly^{A21}Gln^{B3}Glu^{B30}-human insulin, N^{εB29}-tetradecanoyl-Gly^{A21}Gln^{B3}Glu^{B30}-human insulin, N^{εB29}-decanoyl-Gly^{A21}Gln^{B3}Glu^{B30}-human insulin, N^{εB29}-dodecanoyl-Gly^{A21}Gln^{B3}Glu^{B30}-human insulin, N^{εB29}-tridecanoyl-Ala^{A21}Glu^{B30}-human insulin, N^{εB29}-tetradecanoyl-Ala^{A21}Glu^{B30}-human insulin, N^{εB29}-decanoyl-Ala^{A21}Glu^{B30}-human insulin, N^{εB29}-dodecanoyl-Ala^{A21}Glu^{B30}-human insulin, N^{εB29}-tridecanoyl-Ala^{A21}Glu^{B30}-human insulin, N^{εB29}-tetradecanoyl-Ala^{A21}Gln^{B3}Glu^{B30}-human insulin, N^{εB29}-decanoyl-Ala^{A21}Gln^{B3}Glu^{B30}-human insulin N^{εB29}-dodecanoyl-Ala^{A21}Gln^{B3}Glu^{B30}-human insulin. In particular embodiments, the glycosylated insulin analogue comprises at least one N-glycan as disclosed herein attached to the asparagine residue comprising an N-linked glycosylation site.

In particular embodiments, an insulin analogue of the present disclosure comprises the mutations and/or chemical modifications of one of the following insulin analogues: N^{εB29}-tridecanoyl-Gln^{B3} Glu^{B30}-human insulin, N^{εB29}-tetradecanoyl-Gln^{B3}Glu^{B30}-human insulin, N^{εB29}-decanoyl-Gln^{B3}Glu^{B30}-human insulin, N^{εB29}-dodecanoyl-Gln^{B3}Glu^{B30}-human insulin. In particular embodiments, the glycosylated insulin analogue further includes at least one N-glycan as disclosed herein attached to the asparagine residue comprising an N-linked glycosylation site.

In particular embodiments, the in vitro glycosylated or in vivo N-glycosylated insulin analogue comprises the mutations and/or chemical modifications of one of the following insulin analogues: N^{εB29}-formyl-human insulin, N^{αB1}-formyl-human insulin, N^{αA1}-formyl-human insulin, N^{εB29}-formyl-N^{αB1}-formyl-human insulin, N^{εB29}-formyl-N^{αA1}-formyl-human insulin, N^{αA1}-formyl-N^{αB1}-formyl-human insulin, N^{εB29}-formyl-N^{αA1}-formyl-N^{αB1}-formyl-human insulin. In particular embodiments, the glycosylated insulin analogue further includes at least one N-glycan as disclosed herein attached to the asparagine residue comprising an N-linked glycosylation site.

In particular embodiments, the in vitro glycosylated or in vivo N-glycosylated insulin analogue comprises the mutations and/or chemical modifications of one of the following insulin analogues: N^{εB29}-acetyl-human insulin, N^{αB1}-acetyl-human insulin, N^{αA1}-acetyl-human insulin, N^{εB29}-acetyl-N^{αB1}-acetyl-human insulin, N^{εB29}-acetyl-N^{αA1}-acetyl-human insulin, N^{αA1}-acetyl-N^{αB1}-acetyl-human insulin, N^{εB29}-acetyl-N^{αA1}-acetyl-N^{αB1}-acetyl-human insulin. In particular embodiments, the glycosylated insulin analogue further includes at least one N-glycan as disclosed herein attached to the asparagine residue comprising an N-linked glycosylation site.

In particular embodiments, the in vitro glycosylated or in vivo N-glycosylated insulin analogue comprises the mutations and/or chemical modifications of one of the following insulin analogues: N^{εB29}-propionyl-human insulin, N^{αB1}-propionyl-human insulin, N^{αA1}-propionyl-human insulin, N^{εB29}-acetyl-N^{αB1}-propionyl-human insulin, N^{εB29}-propionyl-N^{αA1}-propionyl-human insulin, N^{αA1}-propionyl-N^{αB1}-propionyl-human insulin, N^{εB29}-propionyl-N^{αA1}-propionyl-N^{αB1}-propionyl-human insulin. In particular embodiments, the glycosylated insulin analogue comprises at least one N-glycan as disclosed herein attached to the asparagine residue comprising an N-linked glycosylation site.

In particular embodiments, an insulin analogue of the present disclosure comprises the mutations and/or chemical modifications of one of the following insulin analogues: N^{εB29}-butyryl-human insulin, N^{αB1}-butyryl-human insulin, N^{αA1}-butyryl-human insulin, N^{εB29}-butyryl-N^{αB1}-butyryl-human insulin, N^{εB29}-butyryl-N^{αA1}-butyryl-human insulin, N^{αA1}-butyryl-N^{αB1}-butyryl-human insulin, N^{εB29}-butyryl-N^{αA1}-butyryl-N^{αB1}-butyryl-human insulin. In particular embodiments, the glycosylated insulin analogue further includes at least one N-glycan as disclosed herein attached to the asparagine residue comprising an N-linked glycosylation site.

In particular embodiments, the in vitro glycosylated or in vivo N-glycosylated insulin analogue comprises the mutations and/or chemical modifications of one of the following insulin analogues: N^{εB29}-pentanoyl-human insulin, N^{αB1}-pentanoyl-human insulin, N^{αA1}-pentanoyl-human insulin, N^{εB29}-pentanoyl-N^{αB1}-pentanoyl-human insulin, N^{εB29}-pentanoyl-N^{αA1}-pentanoyl-human insulin, N^{αA1}-pentanoyl-N^{αB1}-pentanoyl-human insulin, N^{εB29}-pentanoyl-N^{αA1}-pentanoyl-N^{αB1}-pentanoyl-human insulin. In particular embodiments, the glycosylated insulin analogue comprises at least one N-glycan as disclosed herein attached to the asparagine residue comprising an N-linked glycosylation site.

In particular embodiments, the in vitro glycosylated or in vivo N-glycosylated insulin analogue comprises the mutations and/or chemical modifications of one of the following insulin analogues: N^{εB29}-hexanoyl-human insulin, N^{αB1}-hexanoyl-human insulin, N^{αA1}-hexanoyl-human insulin, N^{εB29}-hexanoyl-N^{αB1}-hexanoyl-human insulin, N^{εB29}-hexanoyl-N^{αA1}-hexanoyl-human insulin, N^{αA1}-hexanoyl-N^{αB1}-hexanoyl-human insulin, N^{εB29}-hexanoyl-N^{αA1}-hexanoyl-N^{αB1}-hexanoyl-human insulin. In particular embodiments, the glycosylated insulin analogue comprises at least one N-glycan as disclosed herein attached to the asparagine residue comprising an N-linked glycosylation site.

In particular embodiments, the in vitro glycosylated or in vivo N-glycosylated insulin analogue comprises the mutations and/or chemical modifications of one of the following insulin analogues: N^{εB29}-heptanoyl-human insulin, N^{αB1}-heptanoyl-human insulin, N^{αA1}-heptanoyl-human insulin, N^{εB29}-heptanoyl-N^{αB1}-heptanoyl-human insulin, N^{εB29}-heptanoyl-N^{αA1}-heptanoyl-human insulin, N^{αA1}-heptanoyl-N^{αB1}-heptanoyl-human insulin, N^{εB29}-heptanoyl-N^{αA1}-heptanoyl-N^{αB1}-heptanoyl-human insulin. In particular embodiments, the glycosylated insulin analogue further includes at least one N-glycan as disclosed herein attached to the asparagine residue comprising an N-linked glycosylation site.

In particular embodiments, the in vitro glycosylated or in vivo N-glycosylated insulin analogue comprises the mutations and/or chemical modifications of one of the following insulin analogues: N^{αB1}-octanoyl-human insulin, N^{αA1}-octanoyl-human insulin, N^{εB29}-octanoyl-N^{αB1}-octanoyl-human insulin, N^{εB29}-octanoyl-N^{αA1}-octanoyl-human insulin, N^{αA1}-octanoyl-N^{αB1}-octanoyl-human insulin, N^{εB29}-octanoyl-N^{αA1}-octanoyl-N^{αB1}-octanoyl-human insulin. In particular embodiments, the glycosylated insulin analogue further includes at least one N-glycan as disclosed herein attached to the asparagine residue comprising an N-linked glycosylation site.

In particular embodiments, the in vitro glycosylated or in vivo N-glycosylated insulin analogue comprises the mutations and/or chemical modifications of one of the following insulin analogues: N^{εB29}-nonanoyl-human insulin, N^{αB1}-nonanoyl-human insulin, N^{αA1}-nonanoyl-human insulin N^{αA1}-nonanoyl-N^{αB1}-nonanoyl-human insulin, N^{εB29}-nonanoyl-N^{αA1}-nonanoyl-human insulin, N^{αA1}-nonanoyl-N^{αB1}-nonanoyl-human insulin, N^{εB29}-nonanoyl-N^{αA1}-nonanoyl-N^{αB1}-nonanoyl-human insulin. In particular embodiments, the glycosylated insulin analogue further includes at least one N-glycan as disclosed herein attached to the asparagine residue comprising an N-linked glycosylation site.

In particular embodiments, the in vitro glycosylated or in vivo N-glycosylated insulin analogue comprises the mutations and/or chemical modifications of one of the following insulin analogues: N^{εB29}-decanoyl-human insulin, N^{αB1}-decanoyl-human insulin, N^{αA1}-decanoyl-human insulin, N^{εB29}-decanoyl-N^{αB1}-decanoyl-human insulin, N^{εB29}-decanoyl-N^{αA1}-decanoyl-human insulin, N^{αA1}-decanoyl-N^{αB1}-decanoyl-human insulin, N^{εB29}-decanoyl-N^{αA1}-decanoyl-N^{αB1}-decanoyl-human insulin. In particular embodiments, the glycosylated insulin analogue further includes at least one N-glycan as disclosed herein attached to the asparagine residue comprising an N-linked glycosylation site.

In particular embodiments, the in vitro glycosylated or in vivo N-glycosylated insulin analogue comprises the mutations and/or chemical modifications of one of the following insulin analogues: N^{εB28}-formyl-Lys^{B28}Pro^{B29}-human insulin, N^{αB1}-formyl-Lys^{B28}Pro^{B29}-human insulin, N^{αA1}-formyl-Lys^{B28}Pro^{B29}-human insulin, N^{εB28}-formyl-N^{αB1}-formyl-Lys^{B28}Pro^{B29}-human insulin, N^{εB28}-formyl-N^{αA1}-formyl-Lys^{B28}Pro^{B29}-human insulin, N^{αA1}-formyl-N^{αB1}-formyl-Lys^{B28}Pro^{B29}-human insulin, N^{εB28}-formyl-N^{αA1}-formyl-N^{αB1}-formyl-Lys^{B28}Pro^{B29}-human insulin, N^{εB29}-acetyl-Lys^{B28}Pro^{B29}-human insulin, N^{αB1}-acetyl-Lys^{B28}Pro^{B29}-human insulin, N^{αA1}-acetyl-Lys^{B28}Pro^{B29}-human insulin, N^{εB28}-acetyl-N^{αB1}-acetyl-Lys^{B28}Pro^{B29}-human insulin. In particular embodiments, the glycosylated insulin analogue further includes at least one N-glycan as disclosed herein attached to the asparagine residue comprising an N-linked glycosylation site.

In particular embodiments, the in vitro glycosylated or in vivo N-glycosylated insulin analogue comprises the mutations and/or chemical modifications of one of the following insulin analogues: N^{εB28}-acetyl-N^{αA1}-acetyl-Lys^{B28}Pro^{B29}-human insulin, N^{αA1}-acetyl-N^{αB1}-acetyl-Lys^{B28}Pro^{B29}-human insulin, N^{εB28}-acetyl-N^{αA1}-acetyl-N^{αB1}-acetyl-Lys^{B28}Pro^{B29}-human insulin. In particular embodiments, the glycosylated insulin analogue further includes at least one N-glycan as disclosed herein attached to the asparagine residue comprising an N-linked glycosylation site.

In particular embodiments, the in vitro glycosylated or in vivo N-glycosylated insulin analogue comprises the mutations and/or chemical modifications of one of the following insulin analogues: N^{εB28} propionyl-Lys^{B28}Pro^{B29}-human insulin, N^{αB1}-propionyl-Lys^{B28}Pro^{B29}-human insulin, N^{αA1}-propionyl-Lys^{B28}Pro^{B29}-human insulin, N^{εB28}-propionyl-N^{αB1}-propionyl-Lys^{B28}Pro^{B29}-human insulin, N^{εB28}-propionyl-N^{αA1}-propionyl-Lys^{B28}Pro^{B29}-human insulin, N^{αA1}-propionyl-N^{αB1}-propionyl-Lys^{B28}Pro^{B29}-human insulin, N^{εB28}-propionyl-N^{αA1}-propionyl-N^{αB1}-propionyl-Lys^{B28}Pro^{B29}-human insulin. In particular embodiments, the glycosylated insulin analogue comprises at least one N-glycan as disclosed herein attached to the asparagine residue comprising an N-linked glycosylation site.

In particular embodiments, the in vitro glycosylated or in vivo N-glycosylated insulin analogue comprises the mutations and/or chemical modifications of one of the following insulin analogues: N^{εB28}-butyryl-Lys^{B28}Pro^{B29}-human insulin, N^{αB1}-butyryl-Lys^{B28}Pro^{B29}-human insulin, N^{αA1}-butyryl-Lys^{B28}Pro^{B29}-human insulin, N^{εB28}-butyryl-N^{αB1}-butyryl-Lys^{B28}Pro^{B29}-human insulin, N^{εB28}-butyryl-N^{αA1}-butyryl-Lys^{B28}Pro^{B29}-human insulin, N^{αA1}-butyryl-N^{αB1}-butyryl-Lys^{B28}Pro^{B29}-human insulin, N^{εB28}-butyryl-N^{αA1}-butyryl-N^{αB1}-butyryl-Lys^{B28}Pro^{B29}-human insulin. In particular embodiments, the glycosylated insulin analogue further includes at least one N-glycan as disclosed herein attached to the asparagine residue comprising an N-linked glycosylation site.

In particular embodiments, the in vitro glycosylated or in vivo N-glycosylated insulin analogue comprises the mutations and/or chemical modifications of one of the following insulin analogues: N^{εB28}-pentanoyl-Lys^{B28}Pro^{B29}-human insulin, N^{αB1}-pentanoyl-Lys^{B28}Pro^{B29}-human insulin, N^{αA1}-Pentanoyl-Lys^{B28}Pro^{B29}-human insulin, N^{εB28}-pentanoyl-N^{αB1}-pentanoyl-Lys^{B28}Pro^{B29}-human insulin, N^{εB28}-pentanoyl-N^{αA1}-pentanoyl-Lys^{B28}Pro^{B29}-human insulin, N^{αA1}-pentanoyl-N^{αB1}-pentanoyl-Lys^{B28}Pro^{B29}-human insulin, N^{εB28}-pentanoyl-N^{αA1}-pentanoyl-N^{αB1}-pentanoyl-Lys^{B28}Pro^{B29}-human insulin. In particular embodiments, the glycosylated insulin analogue further includes at least one N-glycan as disclosed herein attached to the asparagine residue comprising an N-linked glycosylation site.

In particular embodiments, the in vitro glycosylated or in vivo N-glycosylated insulin analogue comprises the mutations and/or chemical modifications of one of the following insulin analogues: N^{εB28}-hexanoyl-Lys^{B28}Pro^{B29}-human insulin, N^{αB1}-hexanoyl-Lys^{B28}Pro^{B29}-human insulin, N^{αA1}-hexanoyl-Lys^{B28}Pro^{B29}-human insulin, N^{εB28}-hexanoyl-N^{αB1}-hexanoyl-Lys^{B28}Pro^{B29}-human insulin, N^{εB28}-hexanoyl-N^{αA1}-hexanoyl-Lys^{B28}Pro^{B29}-human insulin, N^{αA1}-hexanoyl-N^{αB1}-hexanoyl-Lys^{B28}Pro^{B29}-human insulin, N^{εB28}-hexanoyl-N^{αA1}-hexanoyl-N^{αB1}-hexanoyl-Lys^{B28}Pro^{B29}-human insulin. In particular embodiments, the glycosylated insulin analogue further includes at least one N-glycan as disclosed herein attached to the asparagine residue comprising an N-linked glycosylation site.

In particular embodiments, the in vitro glycosylated or in vivo N-glycosylated insulin analogue comprises the mutations and/or chemical modifications of one of the following insulin analogues: N^{εB28}-heptanoyl-Lys^{B28}Pro^{B29}-human insulin, N^{αB1}-heptanoyl-Lys^{B28}Pro^{B29}-human insulin, N^{αA1}-heptanoyl-Lys^{B28}Pro^{B29}-human insulin, N^{εB28}-heptanoyl-N^{αB1}-heptanoyl-Lys^{B28}Pro^{B29}-human insulin, N^{εB28}-heptanoyl-N^{αA1}-heptanoyl-Lys^{B28}Pro^{B29}-human insulin, N^{αA1}-heptanoyl-N^{αB1}-heptanoyl-Lys^{B28}Pro^{B29}-human insulin, N^{εB28}-heptanoyl-N^{αA1}-heptanoyl-N^{αB1}-heptanoyl-Lys^{B28}Pro^{B29}-human insulin. In particular embodiments, the glycosylated insulin analogue further includes at least one N-glycan as disclosed herein attached to the asparagine residue comprising an N-linked glycosylation site.

In particular embodiments, the in vitro glycosylated or in vivo N-glycosylated insulin analogue comprises the mutations and/or chemical modifications of one of the following insulin analogues: N^{εB28}-octanoyl-Lys^{B28}Pro^{B29}-human insulin, N^{αB1}-octanoyl-Lys^{B28}Pro^{B29}-human insulin, N^{αA1}-octanoyl-Lys^{B28}Pro^{B29}-human insulin, N^{εB28}-octanoyl-N^{αB1}-octanoyl-Lys^{B28}Pro^{B29}-human insulin, N^{εB28}-octanoyl-N^{αA1}-octanoyl-Lys^{B28}Pro^{B29}-human insulin, N^{αA1}-octanoyl-N^{αB1}-octanoyl-Lys^{B28}Pro^{B29}-human insulin, N^{εB28}-octanoyl-N^{αA1}-octanoyl-N^{αB1}-octanoyl-Lys^{B28}Pro^{B29}-human insulin. In particular embodiments, the glycosylated insulin analogue further includes at least one N-glycan as disclosed herein attached to the asparagine residue comprising an N-linked glycosylation site.

In particular embodiments, the in vitro glycosylated or in vivo N-glycosylated insulin analogue comprises the mutations and/or chemical modifications of one of the following insulin analogues: N^{εB28}-nonanoyl-Lys^{B28}Pro^{B29}-human insulin, N^{αB1}-nonanoyl-Lys^{B28}Pro^{B29}-human insulin, N^{αA1}-nonanoyl-Lys^{B28}Pro^{B29}-human insulin, N^{εB28}-nonanoyl-N^{αB1}-nonanoyl-Lys^{B28}Pro^{B29}-human insulin, N^{εB28}-nonanoyl-N^{αA1}-nonanoyl-Lys^{B28}Pro^{B29}-human insulin, N^{αA1}-nonanoyl-N^{αB1}-nonanoyl-Lys^{B28}Pro^{B29}-human insulin, N^{εB28}-nonanoy 1-N^{αA1}-nonanoyl-N^{αB1}-nonanoyl-Lys^{B28}Pro^{B29}-human insulin. In particular embodiments, the glycosylated insulin analogue further includes at least one N-glycan as disclosed herein attached to the asparagine residue comprising an N-linked glycosylation site.

In particular embodiments, the in vitro glycosylated or in vivo N-glycosylated insulin analogue comprises the mutations and/or chemical modifications of one of the following insulin analogues: N^{εB28}-decanoyl-Lys^{B28}Pro^{B29}-human insulin, N^{αB1}-decanoyl-Lys^{B28}Pro^{B29}-human insulin, N^{αA1}-decanoyl-Lys^{B28}Pro^{B29}-human insulin, N^{εB28}-decanoyl-N^{αB1}-decanoyl-Lys^{B28}Pro^{B29}-human insulin, N^{εB28}-decanoyl-N^{αA1}-decanoyl-Lys^{B28}Pro^{B29}-human insulin, N^{αA1}-decanoyl-N^{αB1}-decanoyl-Lys^{B28}Pro^{B29}-human insulin, N^{εB28}-decanoyl-N^{αA1}-decanoyl-N^{αB1}-decanoyl-Lys^{B28}Pro^{B29}-human insulin. In particular embodiments, the glycosylated insulin analogue further includes at least one N-glycan as disclosed herein attached to the asparagine residue comprising an N-linked glycosylation site.

In particular embodiments, the in vitro glycosylated or in vivo N-glycosylated insulin analogue comprises the mutations and/or chemical modifications of one of the following insulin analogues: N^{εB29}-pentanoyl-Gly^{A21}Arg^{B31}Arg^{B32}-human insulin, N^{αB1}-hexanoyl-Gly^{A21}Arg^{B31}Arg^{B32}-human insulin, N^{αA1}-heptanoyl-Gly^{A21}Arg^{B31}Arg^{B32}-human insulin, N^{εB29}-octanoyl-N^{αB1}-octanoyl-Gly^{A21}Arg^{B31}Arg^{B32}-human insulin, N^{εB29}-propionyl-N^{αA1}-propionyl-Gly^{A21}Arg^{B31}Arg^{B32}-human insulin, N^{αA1}-acetyl-N^{αB1}-acetyl-Gly^{A21}Arg^{B31}Arg^{B32}-human insulin, N^{εB29}-formyl-N^{αA1}-formyl-N^{αB1}-formyl-Gly^{A21}Arg^{B31}Arg^{B32}-human insulin, N^{εB29}-formyl-des(B26)-human insulin, N^{αB1}-acetyl-Asp^{B28}-human insulin, N^{εB29}-propionyl-N^{αA1}-propionyl-N^{αB1}-propionyl-Asp^{B1}Asp^{B3}AspB²¹-human insulin, N^{εB29}-pentanoyl-Gly^{A21}-human insulin, N^{αB1}-hexanoyl-Gly^{A21}-human insulin, N^{αA1}-heptanoyl-Gly^{A21}-human insulin, N^{εB29}-octanoyl-N^{αB1}-octanoyl-Gly^{A21}-human insulin, N^{εB29}-propionyl-N^{αA1}-propionyl-Gly^{A21}-human insulin, N^{αA1}-acetyl-N^{αB1}-acetyl-Gly^{A21}-human insulin, N^{εB29}-formyl-N^{αA1}-formyl-N^{αB1}-formyl-Gly^{A21}-human insulin, N^{εB29}-butyryl-des(B30)-human insulin, N^{αB1}-butyryl-des(B30)-human insulin, N^{αA1}-butyryl-des(B30)-human insulin, N^{εB29}-butyryl-N^{αB1}-butyryl-des(B30)-human insulin, N^{εB29}-butyryl-N^{αA1}-butyryl-des(B30)-human insulin, N^{αA1}-butyryl-N^{αB1}-butyryl-des(B30)-human insulin, N^{εB29}-butyryl-N^{αA1}-butyryl-N^{αB1}-butyryl-des(B30)-human insulin. In particular embodiments, the glycosylated insulin analogue further includes at least one N-glycan as disclosed herein attached to the asparagine residue comprising an N-linked glycosylation site or an asparagine residue which had comprised an N-linked glycosylation site when the asparagine residue is at position B28 and glycosylated insulin analogue is desB30.

Therefore, in particular embodiments, the heterodimer or single-chain N-glycosylated insulin analogue comprises an A-chain peptide or B-chain peptide, or analogue thereof comprising 1, 2, 3, 4, 5, or more amino acid substitutions and/or deletions, provided that the insulin molecule further comprises at least one acyl group and at least one N-glycan, e.g., attached at an Asn residue or to NH₂, COOH, SH, or imidizole ring of His. In further embodiments, the heterodimer or single-chain N-glycosylated insulin analogue comprises any one of the aforementioned acylated analogues, or analogue thereof comprising 1, 2, 3, 4, 5, or more amino acid substitutions and/or deletions, provided that the insulin molecule further comprises at least one N-glycan, e.g., attached at an Asn residue or to NH₂, COOH, SH, or imidizole ring of His.

The in vitro glycosylated or in vivo N-glycosylated insulin analogues further includes modified forms of non-human insulins (e.g., porcine insulin, bovine insulin, rabbit insulin, sheep insulin, etc.) that comprise any one of the aforementioned mutations and/or chemical modifications. These and other modified insulin molecules are described in detail in U.S. Pat. Nos. 6,906,028; 6,551,992; 6,465,426; 6,444,641; 6,335,316; 6,268,335; 6,051,551; 6,034,054; 5,952,297; 5,922,675; 5,747,642; 5,693,609; 5,650,486; 5,547,929; 5,504,188; 5,474,978; 5,461,031; and 4,421,685; and in U.S. Pat. Nos. 7,387,996; 6,869,930; 6,174,856; 6,011,007; 5,866,538; and 5,750,497, the entire disclosures of which are hereby incorporated by reference.

In various embodiments, the in vitro glycosylated or in vivo N-glycosylated insulin analogues disclosed herein include the three wild-type disulfide bridges (i.e., one between position 7 of the A-chain and position 7 of the B-chain, a second between position 20 of the A-chain and position 19 of the B-chain, and a third between positions 6 and 11 of the A-chain).

In some embodiments, the in vitro glycosylated or in vivo N-glycosylated insulin analogue is modified and/or mutated to reduce its affinity for the insulin receptor. Without wishing to be bound to a particular theory, it is believed that attenuating the receptor affinity of an insulin molecule through modification (e.g., acylation) or mutation may decrease the rate at which the insulin molecule is eliminated from blood. In some embodiments, a decreased insulin receptor affinity in vitro translates into a superior in vivo activity for the in vitro glycosylated or in vivo N-glycosylated insulin analogue.

### IV. Integration of Insulin Protein Engineering and Glycodesign

a. Pharmacokinetic (PK)/Pharmacodynamic (PD) Improvements

The quality of life for type I diabetics was significantly improved with the introduction of insulin glargine, a once-daily insulin analogue that provides a basal level of insulin in the patient. Due to repetitive blood monitoring and subcutaneous injections that type I diabetics must endure, reduced frequency of injections would be a welcomed advancement in diabetes treatment. Improving the pharmacokinetic profile to meet a once daily injection is greatly sought after for any new insulin treatment. In fact, once-monthly insulin has recently been reported in an animal model (Gupta et al., Proc. Natl. Acad. Sci. USA 107: 13246 (2010); U.S. Pub. Application No. 20090090258818). While many strategies are being pursued to improve the PK profile of insulin, the in vitro glycosylated or in vivo N-glycosylated insulin analogues disclosed herein may provide benefits to the diabetic patient not achievable with other strategies.

Therapeutic proteins have multiple modes of clearance from circulation. Target-mediated clearance is caused by the interaction of the therapeutic protein with the receptor or target molecule. Following engagement with the receptor or target molecule, the ligand-receptor complex is taken into the cell by endocytosis and subsequently targeted to the lysosome for degradation and/or degraded by proteases in the endosome. Another mechanism for clearing proteins from circulation is renal clearance. The glomerulus is the main blood-filtration unit of the kidney. Therapeutic proteins less than about 50 kD, including insulin, are often filtered in the glomerulus to be excreted in urine. Increasing the size of the therapeutic protein to greater than about 50 kD often reduces renal clearance at the glomerulus. Also, circulating proteins with overall negative charge lead to repulsion with membranes in the glomerular filter, thereby reducing clearance. Glycoproteins in circulation that lack terminal sialic acid may also interact with the asialoglycoprotein (Ashwell-Morell) receptor in hepatocyte membranes. Asialylated proteins may demonstrate reduced PK due to lectin-mediated clearance in liver. Another major pathway for protein clearance is proteolytic degradation in circulation. Strategies to reduce degradation mechanisms (See for example, GLP-1 analogues mutated to be resistant to DPIV digestion) can have great impact on overall PK and efficacy profiles. The in vitro conjugation of linear polysialic acid polymers to insulin has been shown to improve (extend) the PK profile of the insulin (Zhang et al., J. Diabetes Sci. Technol. 4: 532 (2010); Timofeev et al., Acta Crystallogr. Sect. F. Struct. Biol. Cryst. Commun. 66: 259 (2010); Bezuglov et al., Bioorg. Khim. 35: 274 (2009); Jain et al., Biochim. Biophys. Acta 1622: 42 (2003)). Sato et al., J. Am. Chem. Soc. 126: 14013 (2004) discloses that insulin analogs having dendridic structures displaying two and three sialyl-N-acetyllactosamines conjugated to a glutamine residue had an extended PK profile. However, construction of various polymers and dendritic structures and in vitro conjugation may be complex and expensive.

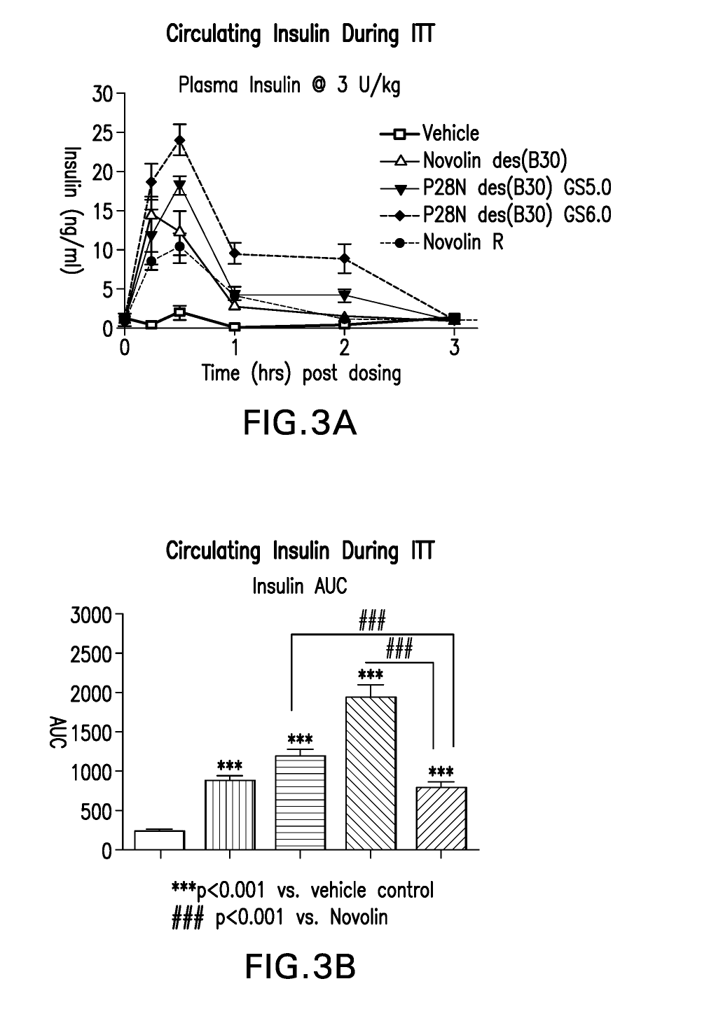

As shown herein, an insulin analogue with a P28N substitution in the B-chain was expressed in a Pichia pastoris strain glycoengineered to produce glycoproteins having N-glycans with a terminal sialic acid residue. Following neuraminidase treatment, insulin with terminal galactose was obtained. The sialylated and galactosylated insulin analogue precursor proteins were treated with endopeptidase LysC to generate des(B30) forms. The des(B30) insulin analogues are active at the insulin receptor but with a reduced efficacy compared to native insulin, and avoids the trypsin-mediated transpeptidation reaction to replace B(Thr30). Recombinant human insulin (NOVOLIN) was also treated with LysC to generate the des(B30) form as a comparator to the glycosylated insulin samples. FIG. 3A and FIG. 3B illustrates the pharmacokinetic properties of the four insulin analogue samples and vehicle (buffer lacking insulin) in an insulin tolerance test (ITT). Both N-glycosylated insulin samples demonstrated an improved or extended PK profile relative to NOVOLIN des(B30). The sialylated insulin sample (GS6.0) and galactosylated insulin sample (GS5.0) demonstrated statistically significant improvements in AUC relative to mature NOVOLIN. Furthermore, the sialic acid-terminated glycoform demonstrated even greater AUC measurements relative to the galactose-terminated glycoform.

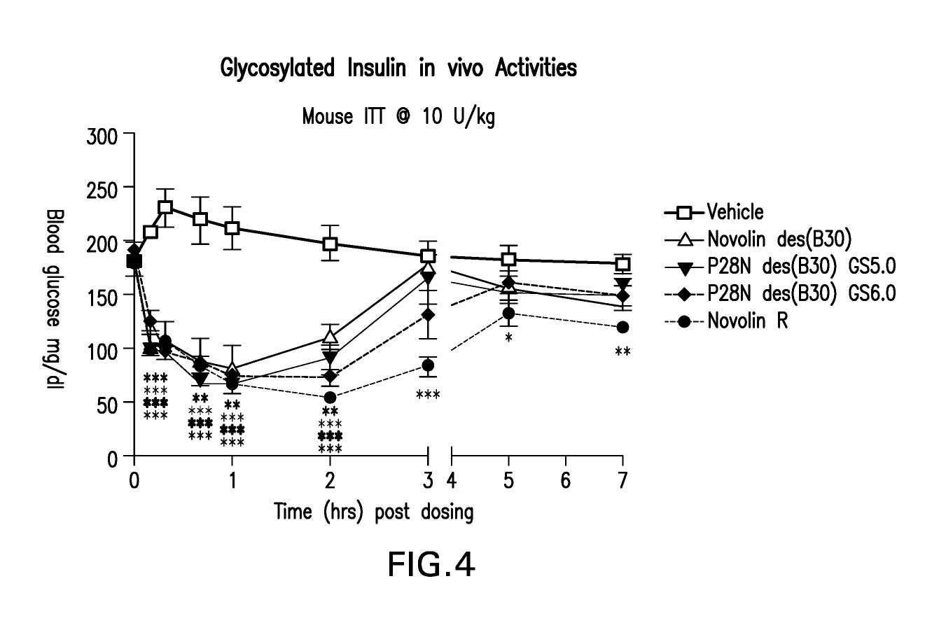

When in vivo glucose levels were monitored in a mouse ITT, both the sialic acid-terminated glycoform and galactose-terminated glycoform retained activity at the insulin receptor (FIG. 4). Unlike the AUC measurements shown in FIG. 3A and FIG. 3B, NOVOLIN des(B30) demonstrated much reduced glucose-lowering activity relative to unprocessed NOVOLIN. Of importance is a difference in formulation buffer compositions between processed and unprocessed NOVOLIN, which may affect the in vivo activity. The formulation buffers for all des(B30) samples were identical, so the comparison of N-glycosylated insulin to NOVOLIN des(B30) revealed an increase in glucose-lowering activity for both N-glycosylated samples. In fact, the sialic acid-terminated glycoform demonstrated the longest glucose-lowering activity of all des(B30) samples, which may be related to improved AUC (Area Under the Curve) measurements. Overall, the data from FIG. 3A and FIG. 3B and FIG. 4 demonstrate the insulin B-chain P28N substitution is not only competent for retaining insulin activity at the insulin receptor but also that the different glycoforms alter the in vivo PK/PD profile of the insulin advantageously.

Further protein engineering and glycodesign may provide in vitro or in vivo glycosylated insulin analogues with further improved or modified PK/PD profiles. For example, adding additional sialylated N-glycans to the insulin analogue may further lower the pI of insulin analogue with an improvement in AUC measurements. In an alternative embodiment, providing an N-glycosylated insulin analogue with an N-glycan linked to the asparagine at position B28 of the B-chain and increasing the amount of sialic acid linked to the N-glycan may also increase AUC. This may be accomplished by adding multi-antennary glycans for trisialylated and tetrasialylated glycoforms. Sialic acid may also be added in an α-2,8 linkage in addition to the α-2,6- and α-2,3-linked sialic acid. Glycoforms other than sialic acid may also improve or modify PK profiles by reducing receptor-mediated clearance or reduced degradation.

Aside from extending protein half-life and increasing AUC, N-glycans, particularly when at the B28 or B29 position of the insulin analogue may increase the rate of bioavailability after subcutaneous injection by reducing ability of the insulin analogues to form hexamers. Thus, N-glycans at these positions may provide rapidly-acting insulin analogues. By the sheer size of an N-glycan (greater than 1-2 kD) or by the addition of negative charge to the N-glycan by sialic acid, N-glycans that give rise to an extremely rapid-acting insulin may be constructed.

Therefore, in particular embodiments, provided is a heterodimer or single-chain N-glycosylated insulin analogue having a modified PK profile and/or PD profile compared to the PK profile and/or PD profile of native insulin comprising any combination of A- and B-chain peptides having a native A-chain, native B-chain, or an amino acid sequence selected from the group of sequences shown by SEQ ID NOs:162 to 254, provided that at least one asparagine residue in the heterodimer or single-chain insulin analogue is attached to an N-glycan comprising at least one terminal sialic acid residue at the non-reducing end. In a further embodiment, provided is a heterodimer or single-chain N-glycosylated insulin analogue having a modified PK profile and/or PD profile compared to the PK and/or PD profile of native insulin comprising a native A-chain peptide and B-chain peptide, or analogue thereof comprising 1, 2, 3, 4, 5, or more amino acid substitutions and/or deletions, provided that the insulin molecule is conjugated to at least one N-glycan comprising at least one terminal sialic acid residue at the non-reducing end, e.g., at that at least one NH₂, COOH, SH, or imidizole ring of His of the molecule is conjugated to an N-glycan comprising at least one terminal sialic acid residue.

b. Altered Binding to IR

The interaction of insulin and the insulin receptor (IR) is of critical importance for glucose uptake. As described above, receptor-mediated endocytosis is one mechanism for insulin clearance. Based on the general concepts of receptor biology, an extremely tight interaction between insulin and IR may lead to an increase in receptor-mediated endocytosis and reduced PK. Alternatively, lower binding affinity to IR may extend PK, but too low of a binding affinity may also reduce glucose uptake. Evolution has balanced these forces for endogenous insulin to generate rapid glucose uptake upon insulin release by the pancreas. However, subcutaneous insulin delivery may require an altered binding relationship. Long-lasting insulin in circulation may require reduced insulin binding to IR to prevent hypoglycemia.

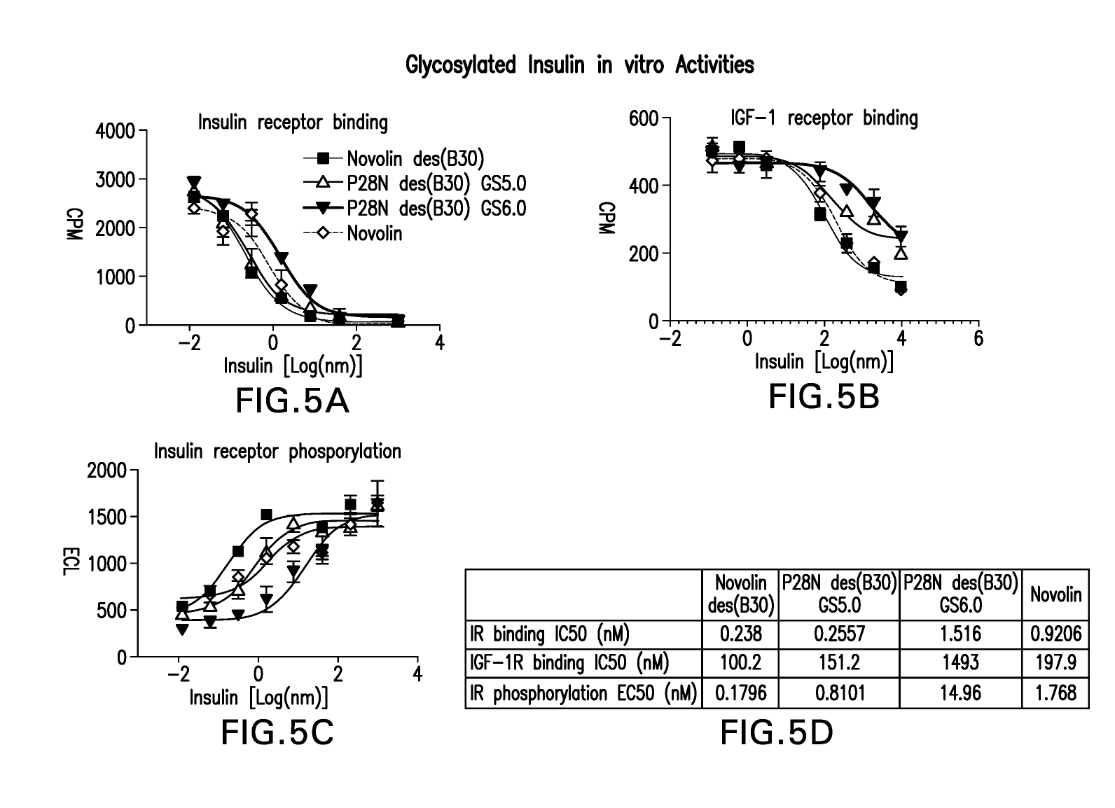

N-glycans provide a means for modulating IR binding. As seen in FIG. 5A, FIG. 5B, FIG. 5C, and FIG. 5D, the N-glycosylated insulin samples demonstrated N-glycan-dependent IR binding profiles. Although the insulin samples having galactose-terminated N-glycans exhibited similar in vitro IR binding as non-glycosylated insulins, the insulin samples having sialic acid-terminated insulin N-glycans had reduced binding activity to IR. Similarly, an in vitro IR signaling assay showed reduced activity of the insulin sample sialic-acid terminated N-glycans relative to the other samples. The sialylated N-glycans extended the PK of the insulin relative to insulin analogues having non-sialylated N-glycans. However, the extended PK is balanced by the reduced binding at the IR. These data demonstrate that the IR binding activity of an N-glycosylated insulin analogue can be modified by the particular glycoform linked to the asparagine at position B28. In light of the examples shown herein, modulating insulin-IR interactions can be accomplished by providing glycosylated insulin analogues in which one or more N-glycans have been added to the molecule by N-linked glycosylation in vivo or by attaching one or more of the N-glycans to the insulin molecule in vitro or a combination of both.

c. Altered Binding to IGF-1R

The insulin-like growth factor-1 (IGF-1) receptor (IGF-1R) is a mitogenic receptor that leads to cell proliferation. Endogenous and therapeutic insulins are known to bind to this receptor. Since many cancer cells utilize the IGF-1R for abnormal cell proliferation, therapeutic insulins are tested for their ability to bind IGF-1R and induce cell proliferation. It is generally considered unfavorable for an insulin analogue to have high IGF-1R binding affinities. Although approved by the FDA, insulin glargine binds IGF-1R with much higher affinity than human insulin. Insulin glargine has been on the market for ten years and to date there does not appear to be any conclusive evidence that patients who use insulin glargine are at an increased risk of cancer. However, studies are ongoing to further understand the cancer risk as patients remain on insulin glargine treatment for extended duration. Due to these concerns, it would be desirable to have an insulin analogue that had an IGF-1R binding affinity that was not significantly greater than the binding affinity of wild-type endogenous human insulin.

Published studies have shown insulin to have a reduced interaction with IGF-1R when it contains a net negative charge at the end of the B-chain (Slieker et al., op. cit.). Therefore, we hypothesized that an N-glycosylated insulin analogue having sialic acid terminated-N-glycans would have reduced IGF-1R binding. As seen in FIG. 5A, FIG. 5B, FIG. 5C, and FIG. 5D, an N-glycosylated insulin analogue that has sialic acid-terminated N-glycans interacts with IGF-1R with even less affinity than NOVOLIN (recombinant human insulin) or an N-glycosylated insulin analogue that has galactose-terminated-N-glycans. Thus, glycosylated insulins comprising sialic acid residues at least one terminus of the N-glycan may provide glycosylated insulin analogues that have an IGF-1R binding affinity that is no greater than the affinity of insulin glargine for the IGF-1R. In particular embodiments, the affinity of the glycosylated insulin analogue with at least one terminus of the N-glycan or glycan is about the same as native insulin or less than native insulin at the IGF-1R.

d. Co-Engagement of Receptors for Liver-Directed Glycosylated Insulin Analogues

The liver has many critical functions in normal physiology, such as protein synthesis, lipid metabolism, detoxification and excretion of metabolites, and carbohydrate transformation. The hepatocyte is the major cell type performing these functions and comprises over 70% of liver mass. The portal vein originates from the gastrointestinal tract and carries about 75% of blood to the liver, the rest from hepatic arteries.

In the postprandial state, glucose levels rise and pancreatic beta cells secrete insulin. The portal vein carries blood glucose and insulin to hepatocytes, whereby the interaction of insulin with the cell surface insulin receptor leads to glucose uptake. Glucose is converted to glycogen when insulin and glucose levels remain high in circulation. The majority of secreted insulin is taken up by hepatocytes by receptor-mediated endocytosis after interaction with the insulin receptor, the rest being filtered out of the blood by kidneys. Alternatively, secreted insulin molecules may continue through the circulatory system to promote glucose uptake in muscle, adipose, or other tissues to support cell metabolism. Following ingestion of the meal, blood glucose levels are reduced through the action of cellular glucose uptake. When glucose levels fall, insulin secretion is reduced, and the lack of insulin receptor signaling in hepatocytes ceases glycogen synthesis. When entering the fasting state, no carbohydrates are ingested, and a low basal level of insulin is secreted by pancreatic beta cells to control blood glucose. Over time, blood glucose levels may fall below normal without food consumption, and pancreatic alpha cells increase secretion of glucagon. Glucagon acts on hepatocytes to stimulate the breakdown of glycogen and the release of glucose to support cellular metabolism. Glycogen stores in the liver are sufficient to act as the primary source of blood glucose in the fasting state for eight to twelve hours. After ingestion of carbohydrates, blood glucose levels reduce secretion of glucagon and increase insulin release to restore the glycogen stores in liver and other tissues.

Endogenous bolus (postprandial) and basal (fasting) insulin act primarily on the liver, with an estimated two- to three-fold excess of insulin activity in the liver relative to peripheral muscle and adipose tissue. Alternatively, the majority of subcutaneously-administered therapeutic insulin engages the insulin receptor on muscle and adipose tissue, with as little as 1% of subcutaneously injected insulin reaching hepatocytes (Canfield et al., Endocrinology 90: 112 (1972)). Results from several studies have been used to argue that insulin controls hepatic glucose production through peripheral actions (e.g., reducing the flow of fatty acids and gluconeogenic substrates to the liver). On the other hand, other studies have demonstrated the additional importance of a direct action of insulin on reducing hepatic glucose production over and above the indirect action of the hormone on peripheral tissues. Furthermore, a substantial body of work has emphasized the ability of portal insulin to significantly increase hepatic glucose uptake after a glucose load. Thus, it is evident that hepatic actions of insulin play a substantial role in reducing postprandial glycemia by (1) more effectively reducing hepatic glucose output, and (2) increasing glucose uptake by the liver. Therefore, targeting therapeutic insulin to the liver would more closely mimic the natural physiology of endogenous insulin (Davis et al., J. Diabetes Complications 15: 227 (2001)). It has been proposed that liver-directed insulin therapy may reduce some of the side effects of current insulin treatment, such as atherosclerosis, cancer, hypoglycemia, and other adverse metabolic effects, that are the result of peripheral hyperinsulinemia (Geho et al., J. Diabetes Sci. Technol. 3: 1451 (2009)). Furthermore, recent data indicates liver-directed insulin (HDV-I) requires <1% of the dose compared to regular insulin required for liver stimulation (Geho et al., op. cit.). The advantages of hepatospecific insulin are two-fold. First, increased insulin action at the liver should limit hepatic glucose output while increasing hepatic glucose uptake. Second, improved postprandial glycemic control could be obtained with reduced systemic insulinemia, thereby reducing the risk of subsequent hypoglycemia (Davis et al., op. cit.).

Due to the importance of insulin activity on hepatocytes and the physiological delivery of insulin to the liver via the portal vein, an in vivo or in vitro glycosylated insulin analogue as disclosed herein may be utilized as the targeting moiety to hepatocytes. The N-glycan may target a protein on the cell surface, such as a receptor or transporter. For hepatocytes, the asialoglycoprotein receptor, biotin receptor, and hepatobiliary ABC transporters are expressed at a higher level relative to other tissues and may represent a receptor for insulin targeting.

Mutating the insulin sequence to enable the addition of an N-glycan in vivo to the insulin may enable the insulin analogue to preferentially target the liver. In the case of in vivo glycosylation or in vitro N-glycosylation in which the glycan has an N-glycan structure, the addition of an N-glycan to the insulin analogue would not require an exogenous linker since an N-glycan is a natural chemical structure that is attached to the molecule. The liver-targeted insulin analogue may incorporate any protein engineering or glycodesign characteristics as described herein. The liver-targeted insulin is comprised of an insulin analogue to which an N-glycan is directly attached via N-linked glycosylation or by conjugation. The insulin may also contain prodrugs or other moieties that extend protein half-life (i.e. PEG). Liver-directed insulin analogues may also be engineered to exhibit reduced potency to the IR and/or fast off rates of the IR and/or protein binding that avoids a slow onset of action.

1. IR and ASGPR

Targeting molecules to the hepatocyte has been used successfully through the asialoglycoprotein receptor (ASGPR) (Ashwell-Morell receptor). This lectin is used mainly by liver cells for the recognition of senescent erythrocytes that have lost the terminal sialic acid residues from the saccharide chain of their glycoproteins and thus reveal the penultimate galactose residues. The ASGPR is expressed on the surface of hepatocytes as well as Kupffer cells. Kupffer cells are specialized macrophages that function aspart of the reticuloendothelial system in the sinusoids of liver to support the innate immune system for complement-coated pathogens and asialylated glycoproteins. Studies have demonstrated the ASGPR selectively binds glycoproteins with terminal galactose, N-acetylgalactosamine (GalNAc), and α-2,6-sialic acid (Steirer et al., J. Biol. Chem. 284: 3777 (2009)). Like most lectins, the strength of the interaction between the ASGPR and the glycan is dictated by the relative binding affinity to a distinct glycan structure and avidity produced by multiple glycan interactions.

Glycosylated insulin analogues may bind both the insulin receptor and the ASGPR, although not necessarily simultaneously, to target the insulin analogue to the liver. Glycosylated insulin analogues that bind to the ASGPR would exhibit increased local concentrations of insulin in the liver relative to peripheral tissues. As a result, insulin receptors may be activated in the liver at higher rates relative to insulin receptors of muscle and adipose tissue. Alternatively, glycosylated insulin analogues that are taken up by endocytosis may retain activity to activate insulin receptor signaling prior to degradation in the lysosome. The relative affinity of a particular glycosylated insulin to the ASGPR and the IR may be modulated for optimal activity. Since Kupffer cells also express ASGPR but do not express the IR, as do hepatocytes, it may be beneficial to target hepatocytes more than Kupffer cells to activate the IR prior to degradation by the ASGPR. This may be accomplished by both protein engineering and glycodesign to modulate the binding affinities towards IR and ASGPR to select the optimal glycosylated insulin analogue molecule that demonstrates a desired in vivo PK/PD profile.

There are several N-glycans that may bind to the ASGPR. For example, N-glycans with a terminal galactose residue may be suitable targets for the ASGPR. Other terminal sugars that are known to bind to the ASGPR are GalNAc and α-2,6 sialic acid. The terminal Gal/GalNAc/α-2,6 sialic acid may be included in a bi-, tri-, or tetra-antennary N-glycan or conjugated glycan with an N-glycan structure to target the glycosylated analogue to the ASGPR. Alternatively, chemically modified sugars or sugar mimetics based on Gal/GalNAc/α-2,6 sialic acid structures may be identified and attached onto an N-glycan to bind the glycosylated insulin analogue to the ASGPR.

Therefore, in particular embodiments, provided is a asialoglycoprotein receptor targeted heterodimer or single-chain N-glycosylated insulin analogue comprising any combination of A- and B-chain peptides having a native A-chain, native B-chain, or an amino acid sequence selected from the group of sequences shown by SEQ ID NOs:162 to 254, provided that at least one asparagine residue in the heterodimer or single-chain insulin analogue is attached to an N-glycan comprising at least one terminal galactose residue at the non-reducing end. In a further embodiment, provided is a asialoglycoprotein receptor targeted heterodimer or single-chain N-glycosylated insulin analogue comprising a native A-chain peptide and B-chain peptide, or analogue thereof comprising 1, 2, 3, 4, 5, or more amino acid substitutions and/or deletions, provided that the insulin molecule is conjugated to at least one N-glycan comprising at least one terminal galactose residue at the non-reducing end, e.g., at that at least one NH₂, COOH, SH, or imidizole ring of His of the molecule is conjugated to an N-glycan comprising at least one terminal galactose residue.

In further embodiments, provided is a asialoglycoprotein receptor targeted heterodimer or single-chain N-glycosylated insulin analogue comprising any combination of A- and B-chain peptides having a native A-chain, native B-chain, or an amino acid sequence selected from the group of sequences shown by SEQ ID NOs:162 to 254, provided that at least one asparagine residue in the heterodimer or single-chain insulin analogue is attached to an N-glycan comprising at least one terminal α-2,6-linked sialic acid residue at the non-reducing end. In a further embodiment, provided is a asialoglycoprotein receptor targeted heterodimer or single-chain N-glycosylated insulin analogue comprising a native A-chain peptide and B-chain peptide, or analogue thereof comprising 1, 2, 3, 4, 5, or more amino acid substitutions and/or deletions, provided that the insulin molecule is conjugated to at least one N-glycan comprising at least one terminal α-2,6-linked sialic acid residue at the non-reducing end, e.g., at that at least one NH₂, COOH, SH, or imidizole ring of His of the molecule is conjugated to an N-glycan comprising at least one terminal α-2,6-linked sialic acid residue.

Therefore, in particular embodiments, provided is a asialoglycoprotein receptor targeted heterodimer or single-chain N-glycosylated insulin analogue comprising any combination of A- and B-chain peptides having a native A-chain, native B-chain, or an amino acid sequence selected from the group of sequences shown by SEQ ID NOs:162 to 254, provided that at least one asparagine residue in the heterodimer or single-chain insulin analogue is attached to an N-glycan comprising at least one terminal GalNAc residue at the non-reducing end. In a further embodiment, provided is a asialoglycoprotein receptor targeted heterodimer or single-chain N-glycosylated insulin analogue comprising a native A-chain peptide and B-chain peptide, or analogue thereof comprising 1, 2, 3, 4, 5, or more amino acid substitutions and/or deletions, provided that the insulin molecule is conjugated to at least one N-glycan comprising at least one terminal GalNAc residue at the non-reducing end, e.g., at that at least one NH₂, COOH, SH, or imidizole ring of His of the molecule is conjugated to an N-glycan comprising at least one galactose residue.

2. IR and Biotin Receptor

Glycosylated insulin analogues may bind both the insulin receptor and the biotin receptor, although not necessarily simultaneously, to target the glycosylated insulin analogue to the liver. Biotin, also called vitamin H or B7, is a water soluble B vitamin. Previous data indicated biotin receptors are located on the surface of liver cells (Vesely et al., Biochem. Biophys. Res. Commun. 143: 913 (1987)). As such, this represents a potential route of hepatic targeting for the glycosylated insulin analogues.

The expression of insulin with a terminal galactose on an N-glycan in competent hosts allows for the oxidation by galactose oxidase (GAO). Biotin, or variants thereof, may be attached to the oxidized galactose moiety, to interactions with endogenous biotin receptors in vivo. Glycosylated insulin analogues that bind to biotin receptors would exhibit increased local concentrations of insulin in the liver relative to peripheral tissues. As a result, insulin receptors may be activated in the liver at higher rates relative to insulin receptors of muscle and adipose tissue. Alternatively, glycosylated insulin analogues that are taken up by endocytosis may retain activity to activate insulin receptor signaling prior to degradation in the lysosome.

3. IR and Hepatobiliary Receptors

Glycosylated insulin analogues may bind both the insulin receptor and hepatobiliary receptors, although not necessarily simultaneously, to target recombinant insulin to the liver. Hepatobiliary receptors, such as the ABC transporters, function to detoxify the blood from chemical substances (Jonker et al., Front Biosci. 14: 4904 (2009)). Previous data has suggested the conjugation of biliverdin and disofenin to liposomes was efficient to generate liver targeting through the hepatobiliary receptors (U.S. Pat. No. 4,603,044, U.S. Pat. No. 4,863,896, U.S. Pat. No. 7,169,410). The expression of a glycosylated insulin analogue with terminal galactose on the N-glycans thereon in competent hosts allows for the oxidation by galactose oxidase (GAO). Biliverdin or disofenin, or variants thereof, may then be attached to the oxidized galactose moiety, to interactions with endogenous hepatobiliary receptors in vivo. Furthermore, other chemicals that interact with hepatobiliary surface proteins may also be conjugated to insulin to enable a liver-directed insulin mechanism. Glycosylated insulin analogues that bind to hepatobiliary receptors may exhibit increased local concentrations of glycosylated insulin analogue in the liver relative to peripheral tissues. As a result, insulin receptors may be activated in the liver at higher rates relative to insulin receptors of muscle and adipose tissue. Alternatively, glycosylated insulin analogue that is endocytosed may retain activity to activate insulin receptor signaling prior to degradation in the lysosome.

4. Long-Acting Liver-Directed Glycosylated Insulin Analogues

The targeting of insulin to the liver by a number of mechanisms, as described above, may be further optimized to reduce the number of doses per day. An desired insulin therapy may mimic endogenous insulin to control blood glucose primarily at the liver, have no addition adverse risks, and be administered no more than once-daily. As described above, liver-directed insulin may exhibit reduced pharmacokinetic properties due to the receptor-mediated clearance mechanisms of the insulin receptor and targeting receptor (e.g. ASGPR, biotin, hepatobiliary). Should the PK characteristics reveal a need for improvement, the liver-directed glycosylated insulin analogues may be further modified with amino acid additions and/or alterations.

One such modification is to retain the physiochemical properties of insulin glargine, which acts as a basal insulin therapy by virtue of its insolubility at neutral pH. The consequence of neutral pH insolubility is a slow resolubilization process in the subcutaneous depot that enables once-a-day injection. The insulin glargine molecule was designed to add two arginine residues at the end of the B-chain and a substitution of asparagine to glycine at the end of the A-chain. These three changes increased the pI of the protein such that it became soluble in low pH formulation buffer but insoluble at physiological pH. These changes may be incorporated into a liver-directed glycosylated insulin analogue. Expression of a glycosylated insulin glargine with one or more galactose- or GalNAc-terminated N-glycans or glycans may provide a long-acting liver-directed (targeted) insulin therapy.

Therefore, in particular embodiments, provided is a long-acting, liver-directed heterodimer or single-chain N-glycosylated insulin analogue comprising a B-chain having the amino acid sequence FVNQHLCGSHLVEALYLVCGERGFFYTNKTRR (SEQ ID NO:27) and an A-chain having the amino acid sequence GIVEQCCTSICSLYQLENYCG (SEQ ID NO:34) wherein at least one asparagine residue in the heterodimer or single-chain insulin analogue is attached to an N-glycan comprising at least one terminal galactose or GalNAc residue at the non-reducing end. In a further embodiment, provided is a long-acting, liver-directed heterodimer or single-chain N-glycosylated insulin analogue comprising a B-chain having the amino acid sequence FVNQHLCGSHLVEALYLVCGERGFFYTNKTRR (SEQ ID NO:27) and an A-chain having the amino acid sequence GIVEQCCTSICSLYQLENYCG (SEQ ID NO:34), or analogue thereof comprising 1, 2, 3, 4, 5, or more amino acid substitutions and/or deletions, provided that the insulin molecule is conjugated to at least one N-glycan comprising at least one terminal galactose or GalNAc residue at the non-reducing end, e.g., at that at least one NH₂, COOH, SH, or imidizole ring of His of the molecule is conjugated to an N-glycan comprising at least one terminal galactose or GalNAc residue.

e. Glucose-Responsive Glycosylated Insulin Analogues

The concept of modulating insulin bioavailability as a function of the physiological blood glucose level by chemical attachment of a sugar moiety to insulin was first introduced in 1979 by Michael Brownlee (Brownlee & Cerami, op. cit.). A major limitation of the concept was toxicity of concanavalin A to which the glycosylated insulin derivative interacted. Since this initial report, many reports have been published on potential improvements for glucose-regulated insulin but no reports to date have attached the sugar via in vivo N-linked glycosylation (Liu et al., Bioconjug. Chem. 8: 664 (1997)).

Since Brownlee's concept in 1979, a number of different strategies have evolved to sequester insulin in an insulin reservoir when blood glucose levels are low. These include the mannose-binding lectin concanavalin A, which was demonstrated to release a bound insulin-sugar complex with high blood glucose concentrations. More recently, U.S. Pat. No. 7,531,191 and International Application Nos. WO2010088261 and WO2010088286, which are incorporated by reference herein, all disclose systems in which microparticles comprising an insulin-saccharide conjugate bound to an exogenous multivalent saccharide-binding molecule (e.g., lectin or modified lectin) can be administered to a patient wherein the amount and duration of insulin-saccharide conjugate released from the microparticle is a function of the serum concentration of glucose. Other strategies include utilizing modified lectins, endogenous receptors, endogenous lectins, and/or sugar-binding proteins. Such examples include the mannose receptor, mannose-binding protein, and DC-SIGN. For example, International Application No. WO2010088294 discloses that when certain insulin-conjugates were modified to include high affinity saccharide ligands they could be made to exhibit PK/PD profiles that responded to saccharide concentration changes even in the absence of an exogenous multivalent saccharide-binding molecule such as Con A. At least 31 human proteins with mannose-binding properties are known. The larger C-type lectin family encompasses at least 60 human proteins with binding to various sugar moieties. Some of these C-type lectin family members exhibit unknown functions and would also likely serve as an endogenous binding partner for glucose-responsive insulin.

Glucose-responsive insulin is one therapeutic mechanism that may mimic the physiologic pulsation of endogenous insulin release. A major stimulus that triggers insulin release from pancreatic beta cells is high blood glucose. In a similar mechanism, therapeutic glycosylated insulin that is released from protected pools into circulation by high glucose concentrations may function in an oscillatory fashion.

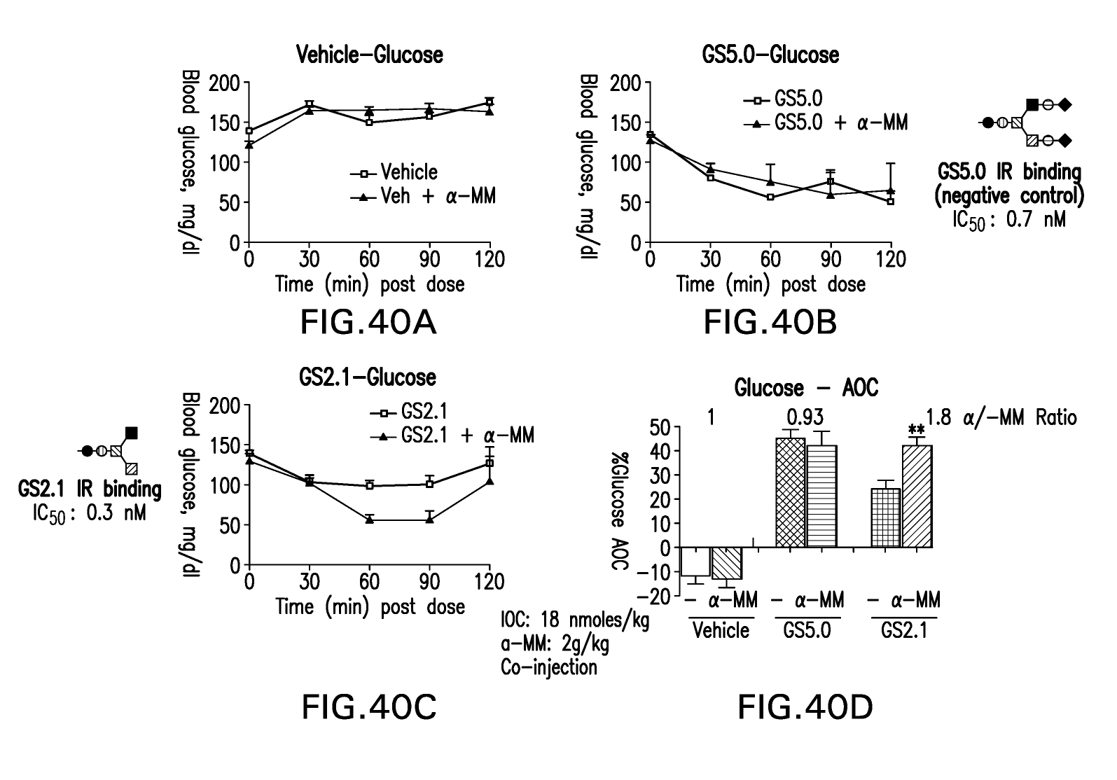

Various N-glycans, for example as shown in FIG. 2, which when linked to an insulin or insulin analogue may function to bind endogenous proteins in a manner that supports a glucose-responsive insulin therapy. Modifying the insulin amino acid sequence to include at least one N-linked glycosylation site may enable the in vivo production of N-glycosylated insulin analogues that are sensitive to serum levels of glucose. N-glycans terminating in terminal mannose or GlcNAc residues may provide glucose-responsive N-linked glycosylated insulin analogues since the main sugars known to interact with mannose-binding domains of human proteins are mannose and GlcNAc sugar residues. As shown in FIG. 40A, FIG. 40B, FIG. 40C, and FIG. 40D, an N-glycosylated insulin analogue with a Man₃GlcNAc₂ glycan structure linked to the asparagine at position B28 rendered the insulin analogue responsive to α-methylmannose, a chemical used to disrupt mannose lectin interactions. In further embodiments, the glycans may further include one or more fucose residues.

Wild-type Pichia pastoris produces N-glycans with high mannose structures, beta-mannose linkages, phosphomannose, and alpha-1,6 mannose linkages that may prove useful for constructing glucose-responsive glycosylated insulin analogues. The N-glycans may be further altered to exclude beta-1,2-mannose, phosphomannose, and alpha-1,6 mannose. Additionally, N-glycans are initially capped with terminal glucose, which is removed upon maturation in the endoplasmic reticulum. Such glucose-terminated structures may also be included in a glycosylated insulin analogue. Particular N-glycans structures that may be included in a glucose-responsive glycosylated insulin analogue include but are not limited to paucimannose (Man₃GlcNAc₂), Man₅GlcNAc₂, Man₆GlcNAc₂, Man₇GlcNAc₂, Man₈GlcNAc₂, Man₉GlcNAc₂, and Man₁₀GlcNAc₂ N-glycans or glycans; Man₃GlcNAc₂ N-glycans or glycans comprising at least one terminal GlcNAc, Gal, or sialic acid residue; GlcNAcMan₅GlcNAc₂, GalGlcNAcMan₅GlcNAc₂, GlcNAcMan₅GlcNAc₂ with core fucose, GlcNAc-Man₅ with core fucose, Man₅ with core fucose, terminal GlcNAc with 1,3 fucose, and Man₅-NANA hybrid. In particular embodiments, the glycosylated insulin analogue comprises at least one N-glycan having at least one terminal mannose residue. In further embodiments, the glycosylated insulin analogue comprises only paucimannose or high mannose N-glycans. In further embodiments, the glycosylated insulin analogue comprises at least one N-glycan selected from structures 43, 51, 105, and 106.

The insulin analogue to which an N-glycan is attached and functions as a glucose-responsive therapy may therefore have the following properties.

The in vivo N-glycosylated or in vitro glycosylated insulin analogue may or may not include one or more additional amino acid substitutions relative to human insulin, a currently marketed insulin analogue, a single chain insulin polypeptide, and may further include analogues containing a hydrophilic polymer such as PEG or a hydrophobic polymer such as a fatty acid, or a prodrug moiety. The oligosaccharide units may contain mannose units and may include both natural and non-natural sugars. The glycosylated insulin analogues may contain one or more one or more N-glycans. The glycosylated insulin analogues may also be prepared synthetically such that the glycan with an N-glycan structure is attached to the peptide sequence using an in vitro reaction. In particular embodiments, the glucose-responsive insulin analogue may contain natural and unnatural non-mannose containing oligosaccharides that enhance clearance through a receptor other than a mannose receptor.

Many endogenous mannose-binding proteins function to support innate immunity. The endogenous sugar-binding proteins complexed with a glycosylated insulin therapy would likely retain the innate immune functions to bind high mannose proteins or pathogens, on top of being responsive to blood glucose. Therefore, targeting the proper sugar-binding protein is important, as well as the type of glycan that interacts with the protein. Screening N-linked and synthetic glycan structures for glucose-responsive properties with reduced side effects may be tested.

Therefore, in particular embodiments, provided is a glucose-responsive heterodimer or single-chain N-glycosylated insulin analogue comprising any combination of A- and B-chain peptides having a native A-chain, native B-chain, or an amino acid sequence selected from the group of sequences shown by SEQ ID NOs:162 to 254, provided that at least one asparagine residue in the heterodimer or single-chain insulin analogue is attached to an N-glycan comprising at least one terminal mannose residue at the non-reducing end. In a further embodiment, a glucose-responsive heterodimer or single-chain N-glycosylated insulin analogue comprising a native A-chain peptide and B-chain peptide, or analogue thereof comprising 1, 2, 3, 4, 5, or more amino acid substitutions and/or deletions, provided that the insulin molecule is conjugated to at least one N-glycan comprising at least one terminal mannose residue at the non-reducing end, e.g., at that at least one NH₂, COOH, SH, or imidizole ring of His of the molecule is conjugated to an N-glycan comprising at least one terminal mannose residue.

f. Long-Acting Glucose-Responsive Glycosylated Insulin Analogues

The function of glucose-responsive insulin, as described above, may be further optimized to reduce the number of doses per day. As described above, glucose-responsive insulin may exhibit reduced pharmacokinetic properties due to the receptor-mediated clearance mechanisms of the insulin receptor and targeting receptor (i.e. mannose receptor, mannose-binding protein, DC-SIGN). Should the PK characteristics reveal a need for improvement, the glucose-responsive glycosylated insulin protein may be further modified with amino acid additions and/or alterations.

One means is to retain the physiochemical properties of insulin glargine, which acts as a basal insulin therapy by virtue of its insolubility at neutral pH. The consequence of neutral pH insolubility is a slow resolubilization process in the subcutaneous depot that enables once-a-day injection. Insulin glargine was modified to include two arginine residues at the end of the B-chain and substitute asparagine for glycine at the end of the A-chain. These three changes increase the pI of the protein such that it is soluble in low pH formulation buffer but insoluble at the physiological pH. These changes can be incorporated into a glucose-responsive glycosylated insulin strategy as disclosed herein by modifying the A- or B-chain to include at least one N-linked glycosylation site. For example, in one embodiment, the B-chain has the amino acid sequence FVNQHLCGSHLVEALYLVCGERGFFYTNKTRR (SEQ ID NO:27) and the A-chain has the amino acid sequence GIVEQCCTSICSLYQLENYCG (SEQ ID NO:34). Expression of the insulin precursor gene encoding these sequences in a host capable of producing N-linked glycosylation as disclosed herein may provide a long-acting glucose-responsive insulin. Alternatively, the insulin analogue may be glycosylated in vitro with a glycan with an N-glycan structure.

Therefore, in particular embodiments, provided is a long-acting, glucose-responsive heterodimer or single-chain N-glycosylated insulin analogue comprising a B-chain having the amino acid sequence FVNQHLCGSHLVEALYLVCGERGFFYTNKTRR (SEQ ID NO:27) and an A-chain having the amino acid sequence GIVEQCCTSICSLYQLENYCG (SEQ ID NO:34) wherein at least one asparagine residue in the heterodimer or single-chain insulin analogue is attached to an N-glycan comprising at least one terminal mannose residue at the non-reducing end. In a further embodiment, provided is a long-acting, glucose-responsive heterodimer or single-chain N-glycosylated insulin analogue comprising a B-chain having the amino acid sequence FVNQHLCGSHLVEALYLVCGERGFFYTNKTRR (SEQ ID NO:27) and an A-chain having the amino acid sequence GIVEQCCTSICSLYQLENYCG (SEQ ID NO:34), or analogue thereof comprising 1, 2, 3, 4, 5, or more amino acid substitutions and/or deletions, provided that the insulin molecule is conjugated to at least one N-glycan comprising at least one terminal mannose residue at the non-reducing end, e.g., at that at least one NH₂, COOH, SH, or imidizole ring of His of the molecule is conjugated to an N-glycan comprising at least one terminal mannose residue.

g. Glycosylated Insulin Analogue Interactions with Human Lectins

Lectins are proteins that bind to carbohydrate moieties. There are multiple types of lectins, including the C-type, I-type, P-type, galectin, and pentraxin groups, that are involved in intra- and intercellular glycan routing and act as defense molecules (Kaltner & Gabius, Adv. Exp. Med. Biol. 491: 79 (2001)). The C-type, Siglec, and galectin groups are pattern recognition receptors (Dam & Brewer, Glycobiology 20: 270 (2010)). The most widely characterized lectins of the I-type are known as Siglecs, or sialic acid-binding lectins that interact with terminal α-2,3/α-2,6/α-2,8 sialic acid (Crocker et al., Nature Reviews Immunology 7: 255 (2007)). The galectins have specificities towards β-gal and LacNAc moieties (Dam & Brewer, op. cit.). The C-type lectins are calcium-dependent proteins that are divided into the following two families: mannose (Man)-specific with binding to Man and/or fucose-terminated glycans; galactose (Gal)-specific with binding to Gal and/or GalNAc (Dam & Brewer, op. cit.). The affinity for C-type lectins increases with polyvalent display, such that the specific affinity and avidity to a glycan structure is important.

Targeting of a therapeutic protein, molecule, or drug to a lectin by way of synthetic carbohydrate structures in order to improve efficacy has been reported (Bernardes et al., Org. Biomol. Chem. 8: 4987-4996 (2010); Lepenies et al., Curr. Opin. Chem. Biol. 14: 404 (2010)). Additionally, synthetic or semi-synthetic glycans have also been shown to affect interactions with lectins and the subsequent biodistribution of the glycoprotein in vivo (Andre et al., Biol. Chem. 390: 557 (2009)). Man-specific C-type lectins have been used to target vaccines to antigen-presenting cells, such as the mannose receptor, DEC-205, Endo-180, phospholipase A2 receptor, DC-SIGN, DC-SIGNR, LSECtin, BDCA-2, and dectin-1 (Keler et al., Expert. Opin. Biol. Ther. 4: 1953 (2004)). The following receptor-ligand relationships have been identified for Man-specific C-type lectins: mannose receptor—mannose, fucose, and GlcNAc; dectin-1—β-glucan; DC-SIGN—mannan (high mannose such as Man6/7/8/9), sialylated lewis structures, agalactosylated glycans (GlcNAc₁Man₃GlcNAc₂, GlcNAc₂Man₃GlcNAc₂, GlcNAc₃Man₃GlcNAc₂, GlcNAc₂Man₃GlcNAc₂fucose, GalGlcNAc₂Man₃GlcNAc₂, GalGlcNAc₂Man₃GlcNAc₂fucose; DC-SIGNR—mannan (high mannose such as Man 6/7/8/9), GlcNAc₂Man₃GlcNAc₂, GlcNAc₂Man₃GlcNAc₂fucose (Keler et al., op. cit.; Yabe et al., FEBS J. 277: 4010 (2010)). Such structures may be suitable moieties to attach to an insulin analogue to provide an glycosylated insulin analogue with a glucose-responsive profile in vivo.

Another lectin that interacts with mannose glycans is the mannose-binding lectin (MBL), also known as the mannan-binding lectin or mannose-binding protein. This is a secreted protein that circulates in blood to support the innate immune system. MBL also functions to initiate the lectin-mediated complement cascade. Interestingly, MBL levels are highly variable and MBL deficiency occurs in more than one-third of the human population and may vary in diabetic patients (Fernandez-Real et al., Diabetologia 49: 2402 (2006); Fortpied et al., Diabetes Metab Res. Rev. 26: 254 (2010)). As protein glycation increases with high blood sugar, it has been postulated that MBL may exhibit altered binding to mannose, fructose, and fructolysine and contribute to complement activation and a role in the pathogenesis of diabetes (Fortpied et al., op. cit.). Additionally, the binding of mannose glycans to MBL was shown to be responsive to blood glucose levels (Ilyas et al., Immunobiology 216: 126-131 (2011); on line Jul. 1, 2010). As such, targeting a glycosylated insulin to MBL and have it function with a glucose-responsive activity may be obtained using N-glycans containing mannose, particularly, a terminal mannose, for example, such as those outlined in section III and FIG. 2.

The other main class of C-type lectin the Gal-specific lectins. Such receptors in this class are the asialoglycoprotein H1 and H2 receptor (ASGPR) and the macrophage galactose-type lectin (MGL). The ASGPR binds preferentially to tri- or tetra-antennary glycans with terminal galactose and GalNAc; alternatively MGL binds preferentially to glycans with terminal GalNAc (van Vliet et al., Trends Immunol. 29: 83 (2008)). Since the ASGPR is located on the surface of hepatocytes while the MGL is found on immature dendritic cells and macrophages, it may be most preferential to utilize tri- or tetraantennary glycans with terminal galactose for liver-directed activity, but terminal GalNAc should also be tested for in vivo activity.

h. Glycosylated Insulin Analogue PD and PK

In the various embodiments disclosed herein, the pharmacokinetic and/or pharmacodynamic behavior of an in vivo N-glycosylated or in vitro glycosylated insulin analogue as disclosed herein may be modified by variations in the serum concentration of a saccharide, including but not limited to glucose and alpha-methyl-mannose.

For example, from a pharmacokinetic (PK) perspective, the serum concentration curve may shift upward when the serum concentration of the saccharide (e.g., glucose) increases or when the serum concentration of the saccharide crosses a threshold (e.g., is higher than normal glucose levels).

In particular embodiments, the serum concentration curve of an in vivo N-glycosylated or in vitro glycosylated insulin analogue as disclosed herein is substantially different when administered to the mammal under fasted and hyperglycemic conditions. As used herein, the term “substantially different” means that the two curves are statistically different as determined by a student t-test (p<0.05). As used herein, the term “fasted conditions” means that the serum concentration curve was obtained by combining data from five or more fasted non-diabetic individuals. In particular embodiments, a fasted non-diabetic individual is a randomly selected 18-30 year old human who presents with no diabetic symptoms at the time blood is drawn and who has not eaten within 12 hours of the time blood is drawn. As used herein, the term “hyperglycemic conditions” means that the serum concentration curve was obtained by combining data from five or more fasted non-diabetic individuals in which hyperglycemic conditions (glucose Cmax at least 100 mg/dL above the mean glucose concentration observed under fasted conditions) is induced by concurrent administration of an in vivo or in vitro glycosylated insulin analogue as disclosed herein and glucose.

Concurrent administration of an in vivo N-glycosylated or in vitro glycosylated insulin analogue as disclosed herein and glucose simply requires that the glucose Cmax occur during the period when the glycosylated insulin analogue is present at a detectable level in the serum. For example, a glucose injection (or ingestion) could be timed to occur shortly before, at the same time or shortly after the glycosylated insulin analogue is administered. In particular embodiments, the in vivo N-glycosylated or in vitro glycosylated insulin analogue as disclosed herein and glucose are administered by different routes or at different locations. For example, in particular embodiments, the in vivo N-glycosylated or in vitro glycosylated insulin analogue as disclosed herein is administered subcutaneously while glucose is administered orally or intravenously.

In particular embodiments, the serum Cmax of the in vivo N-glycosylated or in vitro glycosylated insulin analogue as disclosed herein is higher under hyperglycemic conditions as compared to fasted conditions. Additionally or alternatively, in particular embodiments, the serum area under the curve (AUC) of the glycosylated insulin analogue is higher under hyperglycemic conditions as compared to fasted conditions. In various embodiments, the serum elimination rate of the glycosylated insulin analogue is slower under hyperglycemic conditions as compared to fasted conditions. In particular embodiments, the serum concentration curve of the glycosylated insulin analogue can be fit to a two-compartment bi-exponential model with one short and one long half-life. The long half-life may be particularly sensitive to glucose concentration. Thus, in particular embodiments, the long half-life is longer under hyperglycemic conditions as compared to fasted conditions. In particular embodiments, the fasted conditions involve a glucose Cmax of less than 100 mg/dL (e.g., 80 mg/dL, 70 mg/dL, 60 mg/dL, 50 mg/dL, etc.). In particular embodiments, the hyperglycemic conditions involve a glucose Cmax in excess of 200 mg/dL (e.g., 300 mg/dL, 400 mg/dL, 500 mg/dL, 600 mg/dL, etc.). It will be appreciated that other PK parameters such as mean serum residence time (MRT), mean serum absorption time (MAT), etc. could be used instead of or in conjunction with any of the aforementioned parameters.

The normal range of glucose concentrations in humans, dogs, cats, and rats is 60 to 200 mg/dL. One skilled in the art will be able to extrapolate the following values for species with different normal ranges (e.g., the normal range of glucose concentrations in miniature pigs is 40 to 150 mg/dl). In general, glucose concentrations below 50 mg/dL are considered hypoglycemic and glucose concentrations above 200 mg/dL are considered hyperglycemic. In particular embodiments, the PK properties of the in vivo or in vitro glycosylated insulin analogue as disclosed herein may be tested using a glucose clamp method (see Examples) and the serum concentration curve of the in vivo or in vitro glycosylated insulin analogue as disclosed herein may be substantially different when administered at glucose concentrations of 50 and 200 mg/dL, 50 and 300 mg/dL, 50 and 400 mg/dL, 50 and 500 mg/dL, 50 and 600 mg/dL, 100 and 200 mg/dL, 100 and 300 mg/dL, 100 and 400 mg/dL, 100 and 500 mg/dL, 100 and 600 mg/dL, 200 and 300 mg/dL, 200 and 400 mg/dL, 200 and 500 mg/dL, 200 and 600 mg/dL, etc. Additionally or alternatively, the serum Tmax, serum Cmax, mean serum residence time (MRT), mean serum absorption time (MAT) and/or serum half-life may be substantially different at the two glucose concentrations. As discussed below, in particular embodiments, 100 mg/dL and 300 mg/dL may be used as comparative glucose concentrations. It is to be understood however that the present disclosure encompasses each of these embodiments with an alternative pair of comparative glucose concentrations including, without limitation, any one of the following pairs: 50 and 200 mg/dL, 50 and 300 mg/dL, 50 and 400 mg/dL, 50 and 500 mg/dL, 50 and 600 mg/dL, 100 and 200 mg/dL, 100 and 400 mg/dL, 100 and 500 mg/dL, 100 and 600 mg/dL, 200 and 300 mg/dL, 200 and 400 mg/dL, 200 and 500 mg/dL, 200 and 600 mg/dL, etc. Thus, in particular embodiments, the Cmax of the N-glycosylated insulin analogue is higher when administered to the mammal at the higher of the two glucose concentrations (e.g., 300 vs. 100 mg/dL glucose).

In particular embodiments, the Cmax of the in vivo or in vitro glycosylated insulin analogue as disclosed herein is at least 50% (e.g., at least 100%, at least 200% or at least 400%) higher when administered to the mammal at the higher of the two glucose concentrations (e.g., 300 vs. 100 mg/dL glucose). In particular embodiments, the AUC of the in vivo or in vitro glycosylated insulin analogue as disclosed herein is higher when administered to the mammal at the higher of the two glucose concentrations (e.g., 300 vs. 100 mg/dL glucose). In particular embodiments, the AUC of the in vivo or in vitro glycosylated insulin analogue as disclosed herein is at least 50% (e.g., at least e.g., at least 100%, at least 200% or at least 400%) higher when administered to the mammal at the higher of the two glucose concentrations (e.g., 300 vs. 100 mg/dL glucose).

In particular embodiments, the serum elimination rate of the in vivo or in vitro glycosylated insulin analogue as disclosed herein is slower when administered to the mammal at the higher of the two glucose concentrations (e.g., 300 vs. 100 mg/dL glucose). In certain embodiments, the serum elimination rate of the N-glycosylated insulin analogue is at least 25% (e.g., at least 50%, at least 100%, at least 200%, or at least 400%) faster when administered to the mammal at the lower of the two glucose concentrations (e.g., 100 vs. 300 mg/dL glucose).

In particular embodiments, the serum concentration curve of an in vivo or in vitro glycosylated insulin analogue as disclosed herein may be fit using a two-compartment bi-exponential model with one short and one long half-life. The long half-life may be particularly sensitive to glucose concentration. Thus, in particular embodiments, the long half-life is longer when administered to the mammal at the higher of the two glucose concentrations (e.g., 300 vs. 100 mg/dL glucose).

In particular embodiments, the long half-life is at least 50% (e.g., at least 100%, at least 200% or at least 400%) longer when administered to the mammal at the higher of the two glucose concentrations (e.g., 300 vs. 100 mg/dL glucose).

In particular embodiments, provided is a method in which the serum concentration curve of an in vivo or in vitro glycosylated insulin analogue as disclosed herein is obtained at two different glucose concentrations (e.g., 300 vs. 100 mg/dL glucose); the two curves are fit using a two-compartment bi-exponential model with one short and one long half-life; and the long half-lives obtained under the two glucose concentrations are compared. In particular embodiments, this method may be used as an assay for testing or comparing the glucose sensitivity of one or more in vivo or in vitro glycosylated insulin analogue as disclosed herein.

In particular embodiments, provided is a method in which the serum concentration curves of an in vivo N-glycosylated or in vitro glycosylated insulin analogue as disclosed herein and a non-glycosylated version of the insulin are obtained under the same conditions (for example, fasted conditions); the two curves are fit using a two-compartment bi-exponential model with one short and one long half-life; and the long half-lives obtained for the an in vivo N-glycosylated or in vitro glycosylated insulin analogue as disclosed herein and non-glycosylated version are compared. In particular embodiments, this method may be used as an assay for identifying an in vivo or in vitro glycosylated insulin analogue as disclosed herein that are cleared more rapidly than the non-glycosylated version or native insulin.

In particular embodiments, the serum concentration curve of an in vivo or in vitro glycosylated insulin analogue as disclosed herein is substantially the same as the serum concentration curve of a non-glycosylated version of the analogue when administered to the mammal under hyperglycemic conditions. As used herein, the term “substantially the same” means that there is no statistical difference between the two curves as determined by a student t-test (p>0.05). In particular embodiments, the serum concentration curve of the in vivo N-glycosylated or in vitro glycosylated insulin analogue as disclosed herein is substantially different from the serum concentration curve of a non-glycosylated version of the analogue when administered under fasted conditions. In particular embodiments, the serum concentration curve of the an in vivo N-glycosylated or in vitro glycosylated insulin analogue as disclosed herein is substantially the same as the serum concentration curve of a non-glycosylated version of the analogue when administered under hyperglycemic conditions and substantially different when administered under fasted conditions.

In particular embodiments, the hyperglycemic conditions involve a glucose Cmax in excess of 200 mg/dL (e.g., 300 mg/dL, 400 mg/dL, 500 mg/dL, 600 mg/dL, etc.). In particular embodiments, the fasted conditions involve a glucose Cmax of less than 100 mg/dL (e.g., 80 mg/dL, 70 mg/dL, 60 mg/dL, 50 mg/dL, etc.). It will be appreciated that any of the aforementioned PK parameters such as serum Tmax, serum Cmax, AUC, mean serum residence time (MRT), mean serum absorption time (MAT) and/or serum half-life could be compared.

From a pharmacodynamic (PD) perspective, the bioactivity of the an in vivo or in vitro glycosylated insulin analogue as disclosed herein may increase when the glucose concentration increases or when the glucose concentration crosses a threshold, for example, is higher than normal glucose levels. In particular embodiments, the bioactivity of an in vivo N-glycosylated or in vitro glycosylated insulin analogue as disclosed herein is lower when administered under fasted conditions as compared to hyperglycemic conditions.

In particular embodiments, the fasted conditions involve a glucose Cmax of less than 100 mg/dL (e.g., 80 mg/dL, 70 mg/dL, 60 mg/dL, 50 mg/dL, etc.). In particular embodiments, the hyperglycemic conditions involve a glucose Cmax in excess of 200 mg/dL (e.g., 300 mg/dL, 400 mg/dL, 500 mg/dL, 600 mg/dL, etc.).

In particular embodiments, the PD properties of the an in vivo N-glycosylated or in vitro glycosylated insulin analogue as disclosed herein may be tested by measuring the glucose infusion rate (GIR) required to maintain a steady glucose concentration. According to such embodiments, the bioactivity of the an in vivo N-glycosylated or in vitro glycosylated insulin analogue as disclosed herein may be substantially different when administered at glucose concentrations of 50 and 200 mg/dL, 50 and 300 mg/dL, 50 and 400 mg/dL, 50 and 500 mg/dL, 50 and 600 mg/dL, 100 and 200 mg/dL, 100 and 300 mg/dL, 100 and 400 mg/dL, 100 and 500 mg/dL, 100 and 600 mg/dL, 200 and 300 mg/dL, 200 and 400 mg/dL, 200 and 500 mg/dL, 200 and 600 mg/dL, etc. Thus, in particular embodiments, the bioactivity of the an in vivo N-glycosylated or in vitro glycosylated insulin analogue as disclosed herein is higher when administered to the mammal at the higher of the two glucose concentrations (e.g., 300 vs. 100 mg/dL glucose). In certain embodiments, the bioactivity of the N-glycosylated insulin analogue is at least 25% (e.g., at least 50% or at least 100%) higher when administered to the mammal at the higher of the two glucose concentrations (e.g., 300 vs. 100 mg/dL glucose).

The PD behavior for the in vivo or in vitro glycosylated insulin analogue as disclosed herein can be observed by comparing the time to reach minimum blood glucose concentration (Tnadir), the duration over which the blood glucose level remains below a certain percentage of the initial value (e.g., 70% of initial value or 10 T70% BGL), etc. In general, it will be appreciated that any of the PK and PD characteristics discussed herein can be determined according to any of a variety of published pharmacokinetic and pharmacodynamic methods (e.g., see Baudys et al., Bioconjugate Chem. 9: 176-183 (1998) for methods suitable for subcutaneous delivery). It is also to be understood that the PK and/or PD properties may be measured in any mammal (e.g., a human, a rat, a cat, a minipig, a dog, etc.).

In particular embodiments, PK and/or PD properties are measured in a human. In particular embodiments, PK and/or PD properties are measured in a rat. In particular embodiments, PK and/or PD properties are measured in a minipig. In particular embodiments, PK and/or PD properties are measured in a dog. It will also be appreciated that while the foregoing was described in the context of glucose-responsive in vivo N-glycosylated or in vitro glycosylated insulin analogue as disclosed herein, the same properties and assays apply to an in vivo or in vitro glycosylated insulin analogue as disclosed herein that are responsive to other saccharides including exogenous saccharides, e.g., mannose, L-fucose, N-acetyl glucosamine, alpha-methyl mannose, etc. In some aspects, instead of comparing PK and/or PD properties under fasted and hyperglycemic conditions, the PK and/or PD properties may be compared under fasted conditions with and without administration of the exogenous saccharide. It is to be understood that in vivo N-glycosylated or in vitro glycosylated insulin analogues as disclosed herein may be designed that respond to different Cmax values of a given exogenous saccharide.

### V. Host Cells for Making N-Glycosylated Insulin Analogues

In general, bacterial cells such as E. coli and yeast cells such as Saccharomyces cerevisiae or Pichia pastoris have been used for the commercial production of insulin and insulin analogues. For example, Thin et al., Proc. Natl. Acad. Sci. USA 83: 6766-6770 (1986), U.S. Pat. Nos. 4,916,212; 5,618,913; and 7,105,314 disclose producing insulin in Saccharomyces cerevisiae and WO2009104199 discloses producing insulin in Pichia pastoris. Production of insulin in E. coli has been disclosed in numerous publications including Chan et al., Proc. Natl. Acad. Sci. USA 78: 5401-5404 (1981) and U.S. Pat. No. 5,227,293. The advantage of producing insulin in a yeast host is that the insulin molecule is secreted from the host cell in a properly folded configuration with the correct disulfide linkages, which can then be processed enzymatically in vitro to produce an insulin heterodimers. In contrast, insulin produced in E. coli is not processed in vivo. Instead, it is sequestered in inclusion bodies in an improperly folded configuration. The inclusion bodies are harvested from the cells and processed in vitro in a series of reactions to produce an insulin heterodimers in the proper configuration. While insulin is not normally considered a glycoprotein since it lacks N-linked glycosylation sites, when insulin is produced in yeast but not E. coli, a small population of the insulin synthesized appears to be O-glycosylated. These O-glycosylated molecules are considered to be a contaminant in which methods for its removal have been developed (See for example, U.S. Pat. No. 6,180,757 and WO2009104199).

However, for the production of N-glycosylated insulin analogs as disclosed herein lower eukaryotes such as yeast and filamentous fungi are particularly attractive since they can be genetically modified so that they not only express glycoproteins in which the N-glycosylation pattern is mammalian-like or human-like or humanized or in which a particular N-glycan species is predominant. This has been achieved by eliminating selected endogenous glycosylation enzymes and/or supplying exogenous enzymes as described by Gerngross et al., U.S. Pat. No. 7,449,308, the disclosure of which is incorporated herein by reference, and general methods for reducing 0-glycosylation in yeast have been described in International Application No. WO 2007061631.

Thus, in particular aspects of the invention, the host cell is a yeast cell or filamentous fungus host cell. Yeast and filamentous fungi host cells include, but are not limited to Pichia pastoris, Pichia finlandica, Pichia trehalophila, Pichia koclamae, Pichia membranaefaciens, Pichia minuta (Ogataea minuta, Pichia lindneri), Pichia opuntiae, Pichia thermotolerans, Pichia salictaria, Pichia guercuum, Pichia pijperi, Pichia stiptis, Pichia methanolica, Pichia sp., Saccharomyces cerevisiae, Saccharomyces sp., Hansenula polymorpha, Kluyveromyces sp., Kluyveromyces lactis, Yarrowia lipolytica, Hansenula polymorpha, any Kluyveromyces sp., Candida albicans, any Aspergillus sp., Aspergillus nidulans, Aspergillus niger, Aspergillus oryzae, Fusarium sp., Fusarium gramineum, Fusarium venenatum, Physcomitrella patens, Chrysosporium lucknowense, Trichoderma reesei, and Neurospora crassa. In further aspects, the host cell is genetically engineered to produce glycoproteins having predominately a particular N-glycan species.

In particular embodiments, the host cell is a yeast host cell, for example, Saccharomyces cerevisiae, Yarrowia lipolytica, methylotrophic yeast such as Pichia pastoris or Ogataea minuta, mutants thereof, and genetically engineered variants thereof that produce glycoproteins having predominately a particular N-glycan species. In this manner, glycoprotein compositions can be produced in which a specific desired glycoform is predominant in the composition. If desired, additional genetic engineering of the glycosylation can be performed, such that the glycoprotein can be produced with or without core fucosylation. Use of lower eukaryotic host cells such as yeast are further advantageous in that these cells are able to produce relatively homogenous compositions of glycoprotein, such that the predominant glycoform of the glycoprotein may be present as greater than thirty mole percent of the glycoprotein in the composition. In particular aspects, the predominant glycoform may be present in greater than forty mole percent, fifty mole percent, sixty mole percent, seventy mole percent and, most preferably, greater than eighty mole percent of the glycoprotein present in the composition. Such can be achieved by eliminating selected endogenous glycosylation enzymes and/or supplying exogenous enzymes as described by Gerngross et al., U.S. Pat. No. 7,029,872 and U.S. Pat. No. 7,449,308, the disclosures of which are incorporated herein by reference. For example, a host cell can be selected or engineered to be depleted in α1,6-mannosyl transferase activities, which would otherwise add mannose residues onto the N-glycan on a glycoprotein. For example, in yeast such an α1,6-mannosyl transferase activity is encoded by the OCH1 gene and deletion or disruption of expression of the OCH1 gene (och1α) inhibits the production of high mannose or hypermannosylated N-glycans in yeast such as Pichia pastoris or Saccharomyces cerevisiae. (See for example, Gerngross et al. in U.S. Pat. No. 7,029,872; Contreras et al. in U.S. Pat. No. 6,803,225; and Chiba et al. in EP1211310B1 the disclosures of which are incorporated herein by reference). Thus, in one embodiment, the host cell for producing the N-glycosylated insulin or insulin analogues comprises a deletion or disruption of expression of the OCH1 gene (och1α) and includes a nucleic acid molecule encoding an insulin or insulin analogue having at least one N-glycosylation site.

In a further embodiment, the host cell further includes an α1,2-mannosidase catalytic domain fused to a cellular targeting signal peptide not normally associated with the catalytic domain and selected to target the α1,2-mannosidase activity to the ER or Golgi apparatus of the host cell. Passage of recombinant glycoproteins through the ER or Golgi apparatus of the host cell produces recombinant glycoproteins and compositions of the same comprising a Man₅GlcNAc₂ glycoform, for example, N-glycosylated insulin or insulin analogue composition comprising predominantly a Man₅GlcNAc₂ glycoform. For example, U.S. Pat. No. 7,029,872, U.S. Pat. No. 7,449,308, and U.S. Published Patent Application No. 2005/0170452, the disclosures of which are all incorporated herein by reference, disclose lower eukaryote host cells capable of producing recombinant glycoproteins and compositions of the same comprising a Man₅GlcNAc₂ glycoform.

In a further embodiment, the immediately preceding host cell further includes an N-acetylglucosaminyltransferase I (GlcNAc transferase I or GnT I) catalytic domain fused to a cellular targeting signal peptide not normally associated with the catalytic domain and selected to target GlcNAc transferase I activity to the ER or Golgi apparatus of the host cell. Passage of recombinant glycoproteins through the ER or Golgi apparatus of the host cell produces recombinant glycoproteins and compositions of the same comprising a GlcNAcMan₅GlcNAc₂ glycoform, for example a N-glycosylated insulin or insulin analogue composition comprising predominantly a GlcNAcMan₅GlcNAc₂ glycoform. U.S. Pat. No. 7,029,872, U.S. Pat. No. 7,449,308, and U.S. Published Patent Application No. 2005/0170452, the disclosures of which are all incorporated herein by reference, disclose lower eukaryote host cells capable of producing recombinant glycoproteins and compositions of the same comprising a GlcNAcMan₅GlcNAc₂ glycoform. N-glycosylated insulin or insulin analogues produced in the above cells can be treated in vitro with a hexosaminidase to produce N-glycosylated insulin or insulin analogues comprising a Man5GlcNAc₂ glycoform. Alternatively, the N-glycosylated insulin or insulin analogue composition comprising predominantly a GlcNAcMan₅GlcNAc₂ glycoform may be treated in vitro with mannosidase II and then a hexosaminidase to produce a paucimannose N-glycosylated insulin or insulin analogue composition comprising predominantly a Man₃GlcNAc₂ glycoform.

In a further embodiment, the immediately preceding host cell further includes a mannosidase II catalytic domain fused to a cellular targeting signal peptide not normally associated with the catalytic domain and selected to target mannosidase II activity to the ER or Golgi apparatus of the host cell. Passage of recombinant glycoproteins through the ER or Golgi apparatus of the host cell produces recombinant glycoproteins and compositions of the same comprising a GlcNAcMan₃GlcNAc₂ glycoform, for example N-glycosylated insulin or insulin analogue composition comprising predominantly a GlcNAcMan₃GlcNAc₂ glycoform. U.S. Pat. No. 7,029,872 and U.S. Pat. No. 7,625,756, the disclosures of which are all incorporated herein by reference, discloses lower eukaryote host cells that express mannosidase II enzymes and are capable of producing glycoproteins and compositions of the same having predominantly a GlcNAcMan₃GlcNAc₂ glycoform. The N-glycosylated insulin or insulin analogues produced in the above cells can be treated in vitro with a hexosaminidase that removes the terminal GlcNAc residue to produce an N-glycosylated insulin or insulin analogue comprising a Man₃GlcNAc₂ glycoform or the hexosaminidase can be co-expressed in the host cell to produce N-glycosylated insulin or insulin analogues and compositions of the same comprising a Man₃GlcNAc₂ glycoform.

In a further embodiment, the immediately preceding host cell further includes N-acetylglucosaminyltransferase II (GlcNAc transferase II or GnT II) catalytic domain fused to a cellular targeting signal peptide not normally associated with the catalytic domain and selected to target GlcNAc transferase II activity to the ER or Golgi apparatus of the host cell. Passage of recombinant glycoproteins through the ER or Golgi apparatus of the host cell produces recombinant glycoproteins comprising a GlcNAc₂Man₃GlcNAc₂ glycoform, for example N-glycosylated insulin or insulin analogue composition comprising predominantly a GlcNAc₂Man₃GlcNAc₂ glycoform. U.S. Pat. Nos. 7,029,872 and 7,449,308 and U.S. Published Patent Application No. 2005/0170452, the disclosures of which are all incorporated herein by reference, disclose lower eukaryote host cells capable of producing a glycoprotein comprising a GlcNAc₂Man₃GlcNAc₂ glycoform. The N-glycosylated insulin or insulin analogues produced in the above cells can be treated in vitro with a hexosaminidase that removes the terminal GlcNAc residues to produce N-glycosylated insulin or insulin analogues and compositions of the same comprising a Man₃GlcNAc₂ glycoform or the hexosaminidase can be co-expressed in the host cell to produce N-glycosylated insulin or insulin analogues comprising a Man₃GlcNAc₂ glycoform.

In a further embodiment, the immediately preceding host cell further includes a galactosyltransferase catalytic domain fused to a cellular targeting signal peptide not normally associated with the catalytic domain and selected to target galactosyltransferase activity to the ER or Golgi apparatus of the host cell. Passage of recombinant glycoproteins through the ER or Golgi apparatus of the host cell produces recombinant glycoproteins and compositions of the same comprising a GalGlcNAc₂Man₃GlcNAc₂ or Gal₂GlcNAc₂Man₃GlcNAc₂ glycoform, or mixture thereof for example, N-glycosylated insulin or insulin analogue composition comprising predominantly a GalGlcNAc₂Man₃GlcNAc₂ glycoform or Gal₂GlcNAc₂Man₃GlcNAc₂ glycoform or mixture thereof. U.S. Pat. No. 7,029,872 and U.S. Published Patent Application No. 2006/0040353, the disclosures of which are incorporated herein by reference, discloses lower eukaryote host cells capable of producing a glycoprotein and compositions of the same comprising a Gal₂GlcNAc₂Man₃GlcNAc₂ glycoform. The N-glycosylated insulin or insulin analogues and compositions of the same produced in the above cells can be treated in vitro with a galactosidase to produce N-glycosylated insulin or insulin analogues and compositions of the same comprising a GlcNAc₂Man₃GlcNAc₂ glycoform, for example N-glycosylated insulin or insulin analogue composition comprising predominantly a GlcNAc₂Man₃GlcNAc₂ glycoform or the galactosidase can be co-expressed to produce N-glycosylated insulin or insulin analogues comprising the GlcNAc₂Man₃GlcNAc₂ glycoform, for example N-glycosylated insulin or insulin analogue composition comprising predominantly a GlcNAc₂Man₃GlcNAc₂ glycoform.

In a further embodiment, the immediately preceding host cell further includes a sialyltransferase catalytic domain fused to a cellular targeting signal peptide not normally associated with the catalytic domain and selected to target sialyltransferase activity to the ER or Golgi apparatus of the host cell. Passage of recombinant glycoproteins through the ER or Golgi apparatus of the host cell produces recombinant glycoproteins and compositions of the same comprising predominantly a NANA₂Gal₂GlcNAc₂Man₃GlcNAc₂ glycoform or NANAGal₂GlcNAc₂Man₃GlcNAc₂ glycoform or mixture thereof, for example, N-glycosylated insulin or insulin analogue composition comprising predominantly a NANA₂Gal₂GlcNAc₂Man₃GlcNAc₂ glycoform or NANAGal₂GlcNAc₂Man₃GlcNAc₂ glycoform or mixture thereof. For lower eukaryote host cells such as yeast and filamentous fungi, it is useful that the host cell further include a means for providing CMP-sialic acid for transfer to the N-glycan. U.S. Published Patent Application No. 2005/0260729, the disclosure of which is incorporated herein by reference, discloses a method for genetically engineering lower eukaryotes to have a CMP-sialic acid synthesis pathway and U.S. Published Patent Application No. 2006/0286637, the disclosure of which is incorporated herein by reference, discloses a method for genetically engineering lower eukaryotes to produce sialylated glycoproteins. The N-glycosylated insulin or insulin analogues produced in the above cells can be treated in vitro with a neuraminidase to produce N-glycosylated insulin or insulin analogues and compositions of the same comprising predominantly a Gal₂GlcNAc₂Man₃GlcNAc₂ glycoform or mixture thereof or the neuraminidase can be co-expressed in the host cell to produce N-glycosylated insulin or insulin analogues and compositions of the same comprising predominantly a Gal₂GlcNAc₂Man₃GlcNAc₂ glycoform or mixture thereof, for example, N-glycosylated insulin or insulin analogue composition comprising predominantly a Gal₂GlcNAc₂Man₃GlcNAc₂ glycoform or GalGlcNAc₂Man₃GlcNAc₂ glycoform or mixture thereof.

In a further aspect, the above host cell capable of making glycoproteins having a Man₅GlcNAc₂ glycoform can further include a mannosidase III catalytic domain fused to a cellular targeting signal peptide not normally associated with the catalytic domain and selected to target the mannosidase III activity to the ER or Golgi apparatus of the host cell. Passage of recombinant glycoproteins through the ER or Golgi apparatus of the host cell produces recombinant glycoproteins and compositions of the same comprising a Man₃GlcNAc₂ glycoform, for example, an N-glycosylated insulin or insulin analogue composition comprising predominantly a Man₃GlcNAc₂ glycoform. U.S. Pat. No. 7,625,756, the disclosures of which are all incorporated herein by reference, discloses the use of lower eukaryote host cells that express mannosidase III enzymes and are capable of producing glycoproteins and compositions of the same having predominantly a Man₃GlcNAc₂ glycoform.

Any one of the preceding host cells can further include one or more GlcNAc transferase selected from the group consisting of GnT III, GnT IV, GnT V, GnT VI, and GnT IX to produce glycoproteins having bisected (GnT III) and/or multiantennary (GnT IV, V, VI, and IX) N-glycan structures such as disclosed in U.S. Pat. No. 7,598,055 and U.S. Published Patent Application No. 2007/0037248, the disclosures of which are all incorporated herein by reference.

In further embodiments, the host cell that produces glycoproteins that have predominantly GlcNAcMan₅GlcNAc₂ N-glycans further includes a galactosyltransferase catalytic domain fused to a cellular targeting signal peptide not normally associated with the catalytic domain and selected to target galactosyltransferase activity to the ER or Golgi apparatus of the host cell. Passage of recombinant glycoprotein through the ER or Golgi apparatus of the host cell produces recombinant glycoproteins and compositions of the same comprising predominantly the GalGlcNAcMan₅GlcNAc₂ glycoform, for example, an N-glycosylated insulin or insulin analogue composition comprising predominantly a GlcNAcMan₅GlcNAc₂ glycoform.

In a further embodiment, the immediately preceding host cell that produced glycoproteins that have predominantly the GalGlcNAcMan₅GlcNAc₂ N-glycans further includes a sialyltransferase catalytic domain fused to a cellular targeting signal peptide not normally associated with the catalytic domain and selected to target sialyltransferase activity to the ER or Golgi apparatus of the host cell. Passage of recombinant glycoproteins through the ER or Golgi apparatus of the host cell produces recombinant glycoproteins and compositions of the same comprising a NANAGalGlcNAcMan₅GlcNAc₂ glycoform, for example, an N-glycosylated insulin or insulin analogue composition comprising predominantly a GlcNAcMan₅GlcNAc₂ glycoform.

In general yeast and filamentous fungi are not able to make glycoproteins that have N-glycans that include fucose. Therefore, the N-glycans disclosed herein will lack fucose unless the host cell is specifically modified to include a pathway for synthesizing GDP-fucose and a fucosyltransferase. Therefore, in particular aspects where it is desirable to have glycoproteins in which the N-glycan includes fucose, any one of the aforementioned host cells is further modified to include a fucosyltransferase and a pathway for producing fucose and transporting fucose into the ER or Golgi. Examples of methods for modifying Pichia pastoris to render it capable of producing glycoproteins in which one or more of the N-glycans thereon are fucosylated are disclosed in Published International Application No. WO 2008112092, the disclosure of which is incorporated herein by reference. In particular aspects of the invention, the Pichia pastoris host cell is further modified to include a fucosylation pathway comprising a GDP-mannose-4,6-dehydratase, GDP-keto-deoxy-mannose-epimerase/GDP-keto-deoxy-galactose-reductase, GDP-fucose transporter, and a fucosyltransferase. In particular aspects, the fucosyltransferase is selected from the group consisting of α1,2-fucosyltransferase, α1,3-fucosyltransferase, α1,4-fucosyltransferase, and α1,6-fucosyltransferase.

Various of the preceding host cells further include one or more sugar transporters such as UDP-GlcNAc transporters (for example, Kluyveromyces lactis and Mus musculus UDP-GlcNAc transporters), UDP-galactose transporters (for example, Drosophila melanogaster UDP-galactose transporter), and CMP-sialic acid transporter (for example, human sialic acid transporter). Because lower eukaryote host cells such as yeast and filamentous fungi lack the above transporters, it is preferable that lower eukaryote host cells such as yeast and filamentous fungi be genetically engineered to include the above transporters.

Host cells further include Pichia pastoris that are genetically engineered to eliminate glycoproteins having phosphomannose residues by deleting or disrupting expression of one or both of the phosphomannosyltransferase genes PNO1 and MNN4B (See for example, U.S. Pat. Nos. 7,198,921 and 7,259,007; the disclosures of which are all incorporated herein by reference), which in further aspects can also include deleting or disrupting expression of the MNN4A gene. Disruption includes disrupting the open reading frame encoding the particular enzymes or disrupting expression of the open reading frame or abrogating translation of RNAs encoding one or more of the β-mannosyltransferases and/or phosphomannosyltransferases using interfering RNA, antisense RNA, or the like. The host cells can further include any one of the aforementioned host cells modified to produce particular N-glycan structures.

Host cells further include lower eukaryote cells (e.g., yeast such as Pichia pastoris) that are genetically modified to control O-glycosylation of the glycoprotein by deleting or disrupting expression of one or more of the protein O-mannosyltransferase (Dol-P-Man:Protein (Ser/Thr) Mannosyl Transferase genes) (PMTs) (See U.S. Pat. No. 5,714,377; the disclosure of which is incorporated herein by reference) or grown in the presence of Pmtp inhibitors and/or an alpha-mannosidase as disclosed in Published International Application No. WO 2007061631, the disclosure of which is incorporated herein by reference, or both. Disruption includes disrupting the open reading frame encoding the Pmtp or disrupting expression of the open reading frame or abrogating translation of RNAs encoding one or more of the Pmtps using interfering RNA, antisense RNA, or the like. The host cells can further include any one of the aforementioned host cells modified to produce particular N-glycan structures.

Pmtp inhibitors include but are not limited to a benzylidene thiazolidinediones. Examples of benzylidene thiazolidinediones that can be used are 5-[[3,4-bis(phenylmethoxy) phenyl]methylene]-4-oxo-2-thioxo-3-thiazolidineacetic Acid; 5-[[3-(1-Phenylethoxy)-4-(2-phenylethoxy)]phenyl]methylene]-4-oxo-2-thioxo-3-thiazolidineacetic Acid; and 5-[[3-(1-Phenyl-2-hydroxy)ethoxy)-4-(2-phenylethoxy)]phenyl]methylene]-4-oxo-2-thioxo-3-thiazolidineacetic Acid.

In particular embodiments, the function or expression of at least one endogenous PMT gene is reduced, disrupted, or deleted. For example, in particular embodiments the function or expression of at least one endogenous PMT gene selected from the group consisting of the PMT1, PMT2, PMT3, and PMT4 genes is reduced, disrupted, or deleted; or the host cells are cultivated in the presence of one or more PMT inhibitors. In further embodiments, the host cells include one or more PMT gene deletions or disruptions and the host cells are cultivated in the presence of one or more Pmtp inhibitors. In particular aspects of these embodiments, the host cells also express a secreted α-1,2-mannosidase.

PMT gene deletions or disruptions and/or Pmtp inhibitors control O-glycosylation by reducing O-glycosylation occupancy; that is by reducing the total number of O-glycosylation sites on the glycoprotein that are glycosylated. The further addition of an α-1,2-mannosidase that is secreted by the cell controls O-glycosylation by reducing the mannose chain length of the O-glycans that are on the glycoprotein. Thus, combining PMT deletions or disruptions and/or Pmtp inhibitors with expression of a secreted α-1,2-mannosidase controls O-glycosylation by reducing occupancy and chain length. In particular circumstances, the particular combination of PMT deletions or disruptions, Pmtp inhibitors, and α-1,2-mannosidase is determined empirically as particular heterologous glycoproteins (antibodies, for example) may be expressed and transported through the Golgi apparatus with different degrees of efficiency and thus may require a particular combination of PMT deletions or disruptions, Pmtp inhibitors, and α-1,2-mannosidase. In another aspect, genes encoding one or more endogenous mannosyltransferase enzymes are deleted. The deletion(s) can be in combination with providing the secreted α-1,2-mannosidase and/or PMT inhibitors or can be in lieu of providing the secreted α-1,2-mannosidase and/or PMT inhibitors.

Thus, the control of O-glycosylation can be useful for producing particular glycoproteins in the host cells disclosed herein in better total yield or in yield of properly assembled glycoprotein. The reduction or elimination of O-glycosylation appears to have a beneficial effect on the assembly and transport of glycoproteins such as whole antibodies as they traverse the secretory pathway and are transported to the cell surface. Thus, in cells in which O-glycosylation is controlled, the yield of properly assembled glycoproteins such as antibody fragments is increased over the yield obtained in host cells in which O-glycosylation is not controlled.

To reduce or eliminate the likelihood of N-glycans and O-glycans with β-linked mannose residues, which are resistant to a-mannosidases, the recombinant glycoengineered Pichia pastoris host cells are genetically engineered to eliminate glycoproteins having α-mannosidase-resistant N-glycans by deleting or disrupting one or more of the β-mannosyltransferase genes (e.g., BMT1, BMT2, BMT3, and BMT4)(See, U.S. Pat. No. 7,465,577, U.S. Pat. No. 7,713,719, and Published International Application No. WO2011046855, each of which is incorporated herein by reference). The deletion or disruption of BMT2 and one or more of BMT1, BMT3, and BMT4 also reduces or eliminates detectable cross reactivity to antibodies against host cell protein.

In particular embodiments, the host cells do not display Alg3p protein activity or have a deletion or disruption of expression from the ALG3 gene (e.g., deletion or disruption of the open reading frame encoding the Alg3p to render the host cell alg3α) as described in Published U.S. Application No. 20050170452 or US20100227363, which are incorporated herein by reference. Alg3p is Man₅GlcNAc₂-PP-dolichyl alpha-1,3 mannosyltransferase that transferase a mannose residue to the mannose residue of the alpha-1,6 arm of lipid-linked Man₅GlcNAc₂ (FIG. 2, GS 1.3) in an alpha-1,3 linkage to produce lipid-linked Man₆GlcNAc₂ (FIG. 2, GS 1.4), a precursor for the synthesis of lipid-linked Glc₃Man₉GlcNAc₂, which is then transferred by an oligosaccharyltransferase to an asparagine residue of a glycoprotein followed by removal of the glucose (Glc) residues. In host cells that lack Alg3p protein activity, the lipid-linked Man₅GlcNAc₂ oligosaccharide may be transferred by an oligosaccharyltransferase to an aspargine residue of a glycoprotein. In such host cells that further include an α1,2-mannosidase, the Man₅GlcNAc₂ oligosaccharide attached to the glycoprotein is trimmed to a tri-mannose (paucimannose) Man₃GlcNAc₂ structure (FIG. 2, GS 2.1). The Man₅GlcNAc₂ (GS 1.3) structure is distinguishable from the Man₅GlcNAc₂ (GS 2.0) shown in FIG. 2, and which is produced in host cells that express the Man₅GlcNAc₂-PP-dolichyl alpha-1,3 mannosyltransferase (Alg3p).

Therefore, provided is a method for producing an N-glycosylated insulin or insulin analogue and compositions of the same in a lower eukaryote host cell, comprising a deletion or disruption ALG3 gene (alg3α) and includes a nucleic acid molecule encoding an insulin or insulin analogue having at least one N-glycosylation site; and culturing the host cell under conditions for expressing the insulin or insulin analogue to produce the N-glycosylated insulin or insulin analogue having predominantly a Man₅GlcNAc₂ (GS 1.3) structure. In further embodiments, the host cell further expresses an endomannosidase activity (e.g., a full-length endomannosidase or a chimeric endomannosidase comprising an endomannosidase catalytic domain fused to a cellular targeting signal peptide not normally associated with the catalytic domain and selected to target the endomannosidase activity to the ER or Golgi apparatus of the host cell. See for example, U.S. Pat. No. 7,332,299) and/or glucosidase II activity (a full-length glucosidase II or a chimeric glucosidase II comprising a glucosidase II catalytic domain fused to a cellular targeting signal peptide not normally associated with the catalytic domain and selected to target the glucosidase II activity to the ER or Golgi apparatus of the host cell. See for example, U.S. Pat. No. 6,803,225). In particular aspects, the host cell further includes a deletion or disruption of the ALG6 (α1,3-glucosylatransferase) gene (alg6α), which has been shown to increase N-glycan occupancy of glycoproteins in alg3α host cells (See for example, De Pourcq et al., PloSOne 2012; 7(6):e39976. Epub 2012 Jun. 29, which discloses genetically engineering Yarrowia lipolytica to produce glycoproteins that have Man₅GlcNAc₂ (GS 1.3) or paucimannose N-glycan structures). The nucleic acid sequence encoding the Pichia pastoris ALG6 is disclosed in EMBL database, accession number CCCA38426. In further aspects, the host cell further includes a deletion or disruption of the OCH1 gene (och1α).

Further provided is a method for producing an N-glycosylated insulin or insulin analogue and compositions of the same in a lower eukaryote host cell, comprising a deletion or disruption of the ALG3 gene (alg3α) and includes a nucleic acid molecule encoding a chimeric α1,2-mannosidase comprising an α1,2-mannosidase catalytic domain fused to a cellular targeting signal peptide not normally associated with the catalytic domain and selected to target the α1,2-mannosidase activity to the ER or Golgi apparatus of the host cell to overexpress the chimeric α1,2-mannosidase and a nucleic acid molecule encoding the insulin or insulin analogue having at least one N-glycosylation site; and culturing the host cell under conditions for expressing the insulin or insulin analogue to produce the N-glycosylated insulin or insulin analogue having predominantly a Man₃GlcNAc₂ structure. In further embodiments, the host cell further expresses or overexpresses an endomannosidase activity (e.g., a full-length endomannosidase or a chimeric endomannosidase comprising an endomannosidase catalytic domain fused to a cellular targeting signal peptide not normally associated with the catalytic domain and selected to target the endomannosidase activity to the ER or Golgi apparatus of the host cell) and/or a glucosidase II activity (a full-length glucosidase II or a chimeric glucosidase II comprising a glucosidase II catalytic domain fused to a cellular targeting signal peptide not normally associated with the catalytic domain and selected to target the glucosidase II activity to the ER or Golgi apparatus of the host cell). In particular aspects, the host cell further includes a deletion or disruption of the ALG6 gene (alg6α). In further aspects, the host cell further includes a deletion or disruption of the OCH1 gene (och1α) Example 14 shows the construction of an alg3α Pichia pastoris host cell that overexpresses a chimeric α1,2-mannosidase and a full-length endomannosidase. The host cell was shown in Example 15 to produce insulin analogues that have paucimannose N-glycans. Similar host cells may be constructed in other yeast or filamentous fungi.

In further embodiments, the above alg3α host cells may further include additional mammalian or human glycosylation enzymes (e.g., GnT I, GnT II, galactosyltransferase, fucosyltransferase, sialyl transferase) as disclosed previously to produce N-glycosylated insulin or insulin analogue having predominantly particular hybrid or complex N-glycans.

Yield of glycoprotein can in some situations be improved by overexpressing nucleic acid molecules encoding mammalian or human chaperone proteins or replacing the genes encoding one or more endogenous chaperone proteins with nucleic acid molecules encoding one or more mammalian or human chaperone proteins. In addition, the expression of mammalian or human chaperone proteins in the host cell also appears to control O-glycosylation in the cell. Thus, further included are the host cells herein wherein the function of at least one endogenous gene encoding a chaperone protein has been reduced or eliminated, and a vector encoding at least one mammalian or human homolog of the chaperone protein is expressed in the host cell. Also included are host cells in which the endogenous host cell chaperones and the mammalian or human chaperone proteins are expressed. In further aspects, the lower eukaryotic host cell is a yeast or filamentous fungi host cell. Examples of the use of chaperones of host cells in which human chaperone proteins are introduced to improve the yield and reduce or control O-glycosylation of recombinant proteins has been disclosed in Published International Application No. WO 2009105357 and WO2010019487 (the disclosures of which are incorporated herein by reference). Like above, further included are lower eukaryotic host cells wherein, in addition to replacing the genes encoding one or more of the endogenous chaperone proteins with nucleic acid molecules encoding one or more mammalian or human chaperone proteins or overexpressing one or more mammalian or human chaperone proteins as described above, the function or expression of at least one endogenous gene encoding a protein O-mannosyltransferase (PMT) protein is reduced, disrupted, or deleted. In particular embodiments, the function of at least one endogenous PMT gene selected from the group consisting of the PMT1, PMT2, PMT3, and PMT4 genes is reduced, disrupted, or deleted.

The methods disclose herein can use any host cell that has been genetically modified to produce glycoproteins wherein the predominant N-glycan is selected from the group consisting of complex N-glycans, hybrid N-glycans, and high mannose N-glycans wherein complex N-glycans are selected from the group consisting of GlcNAc₍₁₋₄₎Man₃GlcNAc₂, the group consisting of Gal₍₁₋₄₎GlcNAc₍₁₋₄₎Man₃GlcNAc₂, or the group consisting of NANA₍₁₋₄₎Gal₍₁₋₄₎Man₃GlcNAc₂; hybrid N-glycans are selected from the group consisting of GlcNAcMan₅GlcNAc₂, GalGlcNAcMan₅GlcNAc₂, and NANAGalGlcNAcMan₅GlcNAc₂; and high Mannose N-glycans are selected from the group consisting of Man₅GlcNAc₂, Man₆GlcNAc₂, Man₇GlcNAc₂, Man₈GlcNAc₂, and Man₉GlcNAc₂. In a further embodiment, the predominant N-glycan is the paucimannose, Man₃GlcNAc₂.

To increase the N-glycosylation site occupancy on a glycoprotein produced in a recombinant host cell, a nucleic acid molecule encoding a heterologous single-subunit oligosaccharyltransferase, which is capable of functionally suppressing a lethal mutation of one or more essential subunits comprising the endogenous host cell hetero-oligomeric oligosaccharyltransferase (OTase) complex, is overexpressed in the recombinant host cell either before or simultaneously with the expression of the glycoprotein in the host cell. The Leishmania major STT3A protein, Leishmania major STT3B protein, and Leishmania major STT3D protein, are single-subunit oligosaccharyltransferases that have been shown to suppress the lethal phenotype of a deletion of the STT3 locus in Saccharomyces cerevisiae (Naseb et al., Molec. Biol. Cell 19: 3758-3768 (2008)). Naseb et al. (ibid.) further showed that the Leishmania major STT3D protein could suppress the lethal phenotype of a deletion of the WBP1, OST1, SWP1, or OST2 loci. Hese et al. (Glycobiology 19: 160-171 (2009)) teaches that the Leishmania major STT3A (STT3-1), STT3B (STT3-2), and STT3D (STT3-4) proteins can functionally complement deletions of the OST2, SWP1, and WBP1 loci. As shown in PCT/US2011/25878 (Published International Application No. WO2011106389, which is incorporated herein by reference), the Leishmania major STT3D (LmSTT3D) protein is a heterologous single-subunit oligosaccharyltransferases that is capable of suppressing a lethal phenotype of a αstt3 mutation and at least one lethal phenotype of a αwbp1, αost1, αswp1, and αost2 mutation that is shown in the examples herein to be capable of enhancing the N-glycosylation site occupancy of heterologous glycoproteins, for example antibodies, produced by the host cell.

Therefore, in a further aspect of the above, provided is a method for producing an N-glycosylated insulin or insulin analogue in a yeast or filamentous fungus host cell, comprising providing a yeast or filamentous fungus host cell that is genetically engineered to produce glycoproteins that have predominantly a particular N-glycan species and includes a nucleic acid molecule encoding a heterologous single-subunit oligosaccharyltransferase and a nucleic acid molecule encoding an insulin or insulin analogue having at least one N-glycosylation site; and culturing the host cell under conditions for expressing the insulin or insulin analogue having at least one N-glycosylation site to produce the N-glycosylated insulin or insulin analogue.

In a further aspect of the above, provided is a method for producing an N-glycosylated insulin or insulin analogue with a predominant N-glycan species wherein the N-glycosylation site occupancy is greater than 83% in a yeast or filamentous fungus host cell, comprising providing a yeast or filamentous fungus host cell that is genetically engineered to produce glycoproteins that have predominantly a particular N-glycan species and includes a nucleic acid molecule encoding a heterologous single-subunit oligosaccharyltransferase (e.g., the Leishmania major STT3D protein) and a nucleic acid molecule encoding the insulin or insulin analogue having at least one N-glycosylation site; and culturing the host cell under conditions for expressing the insulin or insulin analogue having at least one N-glycosylation site to produce the N-glycosylated insulin or insulin analogue wherein the N-glycosylation site occupancy is greater than 83%. In particular embodiments of the above, the N-glycosylation site occupancy is at least 94%. In further still embodiments, the N-glycosylation site occupancy is at least 99%.

Further provided is a yeast or filamentous fungus host cell genetically engineered to produce N-glycosylated insulin or insulin analogues having predominantly a particular N-glycan species, comprising a first nucleic acid molecule encoding a heterologous single-subunit oligosaccharyltransferase; and a second nucleic acid molecule encoding an insulin or insulin analogue having at least one N-glycosylation site; and wherein the endogenous host cell genes encoding the proteins comprising the oligosaccharyltransferase (OTase) complex are expressed. This includes expression of the endogenous STT3 gene, which in yeast is the STT3 gene.

In general, in the above methods and host cells, the single-subunit oligosaccharyltransferase is capable of functionally suppressing the lethal phenotype of a mutation of at least one essential protein of the OTase complex. In further aspects, the essential protein of the OTase complex is encoded by the STT3 locus, WBP1 locus, OST1 locus, SWP1 locus, or OST2 locus, or homologue thereof. In further aspects, the for example single-subunit oligosaccharyltransferase is the Leishmania major STT3D protein.

Promoters are DNA sequence elements for controlling gene expression. In particular, promoters specify transcription initiation sites and can include a TATA box and upstream promoter elements. The promoters selected are those which would be expected to be operable in the particular host system selected. For example, yeast promoters are used when a yeast such as Saccharomyces cerevisiae, Kluyveromyces lactis, Ogataea minuta, or Pichia pastoris is the host cell whereas fungal promoters would be used in host cells such as Aspergillus niger, Neurospora crassa, or Tricoderma reesei. Examples of yeast promoters include but are not limited to the GAPDH, AOX1, SEC4, HH1, PMA1, OCH1, GAL1, PGK, GAP, TPI, CYC1, ADH2, PHO5, CUP1, MFα1, FLD1, PMA1, PDI, TEF, RPL10, and GUT1 promoters. Romanos et al., Yeast 8: 423-488 (1992) provide a review of yeast promoters and expression vectors. Hartner et al., Nucl. Acid Res. 36: e76 (pub on-line 6 Jun. 2008) describes a library of promoters for fine-tuned expression of heterologous proteins in Pichia pastoris.

The promoters that are operably linked to the nucleic acid molecules disclosed herein can be constitutive promoters or inducible promoters. An inducible promoter, for example the AOX1 promoter, is a promoter that directs transcription at an increased or decreased rate upon binding of a transcription factor in response to an inducer. Transcription factors as used herein include any factor that can bind to a regulatory or control region of a promoter and thereby affect transcription. The RNA synthesis or the promoter binding ability of a transcription factor within the host cell can be controlled by exposing the host to an inducer or removing an inducer from the host cell medium. Accordingly, to regulate expression of an inducible promoter, an inducer is added or removed from the growth medium of the host cell. Such inducers can include sugars, phosphate, alcohol, metal ions, hormones, heat, cold and the like. For example, commonly used inducers in yeast are glucose, galactose, alcohol, and the like.

Transcription termination sequences that are selected are those that are operable in the particular host cell selected. For example, yeast transcription termination sequences are used in expression vectors when a yeast host cell such as Saccharomyces cerevisiae, Kluyveromyces lactis, or Pichia pastoris is the host cell whereas fungal transcription termination sequences would be used in host cells such as Aspergillus niger, Neurospora crassa, or Tricoderma reesei. Transcription termination sequences include but are not limited to the Saccharomyces cerevisiae CYC transcription termination sequence (ScCYC TT), the Pichia pastoris ALG3 transcription termination sequence (ALG3 TT), the Pichia pastoris ALG6 transcription termination sequence (ALG6 TT), the Pichia pastoris ALG12 transcription termination sequence (ALG12 TT), the Pichia pastoris AOX1 transcription termination sequence (AOX1 TT), the Pichia pastoris OCH1 transcription termination sequence (OCH1 TT) and Pichia pastoris PMA1 transcription termination sequence (PMA1 TT). Other transcription termination sequences can be found in the examples and in the art.

For genetically engineering yeast, selectable markers can be used to construct the recombinant host cells include drug resistance markers and genetic functions which allow the yeast host cell to synthesize essential cellular nutrients, e.g. amino acids. Drug resistance markers which are commonly used in yeast include chloramphenicol, kanamycin, methotrexate, G418 (geneticin), Zeocin, and the like. Genetic functions which allow the yeast host cell to synthesize essential cellular nutrients are used with available yeast strains having auxotrophic mutations in the corresponding genomic function. Common yeast selectable markers provide genetic functions for synthesizing leucine (LEU2), tryptophan (TRP1 and TRP2), proline (PRO1), uracil (URA3, URA5, URA6), histidine (HIS3), lysine (LYS2), adenine (ADE1 or ADE2), and the like. Other yeast selectable markers include the ARR3 gene from S. cerevisiae, which confers arsenite resistance to yeast cells that are grown in the presence of arsenite (Bobrowicz et al., Yeast, 13:819-828 (1997); Wysocki et al., J. Biol. Chem. 272:30061-30066 (1997)). A number of suitable integration sites include those enumerated in U.S. Pat. No. 7,479,389 (the disclosure of which is incorporated herein by reference) and include homologs to loci known for Saccharomyces cerevisiae and other yeast or fungi. Methods for integrating vectors into yeast are well known (See for example, U.S. Pat. No. 7,479,389, U.S. Pat. No. 7,514,253, U.S. Published Application No. 2009012400, and WO2009/085135; the disclosures of which are all incorporated herein by reference). Examples of insertion sites include, but are not limited to, Pichia ADE genes; Pichia TRP (including TRP1 through TRP2) genes; Pichia MCA genes; Pichia CYM genes; Pichia PEP genes; Pichia PRB genes; and Pichia LEU genes. The Pichia ADE1 and ARG4 genes have been described in Lin Cereghino et al., Gene 263:159-169 (2001) and U.S. Pat. No. 4,818,700 (the disclosure of which is incorporated herein by reference), the HIS3 and TRP1 genes have been described in Cosano et al., Yeast 14:861-867 (1998), HIS4 has been described in GenBank Accession No. X56180.

The transformation of the yeast cells is well known in the art and may for instance be effected by protoplast formation followed by transformation in a manner known per se. The medium used to cultivate the cells may be any conventional medium suitable for growing yeast organisms. A significant proportion of the secreted N-glycosylated insulin analogue precursor which will be present in the medium in correctly processed form and may be recovered from the medium by various procedures including but not limited to separating the yeast cells from the medium by centrifugation, filtration, or catching the insulin precursor by an ion exchange matrix or by a reverse phase absorption matrix, precipitating the proteinaceous components of the supernatant or filtrate by means of a salt, e.g. ammonium sulphate, followed by purification by a variety of chromatographic procedures, e.g. ion exchange chromatography, affinity chromatography, or the like.

The secreted N-glycosylated insulin analogue precursor may optionally include an N-terminal extension or spacer peptide, as described in U.S. Pat. No. 5,395,922 and European Patent No. 765,395A, both of which are herein specifically incorporated by reference. The N-terminal extension or spacer is a peptide that is positioned between the signal peptide or propeptide and the N-terminus of the B-chain. Following removal of the signal peptide and propeptide during passage through the secretory pathway, the N-terminal extension peptide remains attached to the N-glycosylated insulin precursor. Thus, during fermentation, the N-terminal end of the B-chain is protected against the proteolytic activity of yeast proteases such as DPAP. The presence of an N-terminal extension or spacer peptide may also serve as a protection of the N-terminal amino group during chemical processing of the protein, i.e., it may serve as a substitute for a BOC (t-butyl-oxycarbonyl) or similar protecting group. The N-terminal extension or spacer may be removed from the recovered N-glycosylated insulin precursor by means of a proteolytic enzyme which is specific for a basic amino acid (e.g., Lys) so that the terminal extension is cleaved off at the Lys residue. Examples of such proteolytic enzymes are trypsin, Achromobacter lyticus protease, or Lysobacter enzymogenes endoprotease Lys-C.

After secretion into the culture medium and recovery, the N-glycosylated insulin analogue precursor may be subjected to various in vitro procedures to remove the optional N-terminal extension or spacer peptide and the C-peptide to give an N-glycosylated desB30 insulin. The N-glycosylated desB30 insulin may then be converted into B30 insulin by adding a Thr in position B30. Conversion of the N-glycosylated insulin analogue precursor into a B30 heterodimer by digesting the N-glycosylated insulin analogue precursor with trypsin or Lys-C in the presence of an L-threonine ester followed by conversion of the threonine ester to L-threonine by basic or acid hydrolysis as described in U.S. Pat. No. 4,343,898 or 4,916,212, the disclosures of which are incorporated by reference hereinto. The N-glycosylated desB30 insulin may also be converted into an acylated derivative as disclosed in U.S. Pat. No. 5,750,497 and U.S. Pat. No. 5,905,140, the disclosures of which are incorporated by reference hereinto.

The methods disclosed herein can be adapted for use in mammalian, plant, and insect cells. Examples of animal cells include, but are not limited to, SC-I cells, LLC-MK cells, CV-I cells, CHO cells, COS cells, murine cells, human cells, HeLa cells, 293 cells, VERO cells, MDBK cells, MDCK cells, MDOK cells, CRFK cells, RAF cells, TCMK cells, LLC-PK cells, PK15 cells, WI-38 cells, MRC-5 cells, T-FLY cells, BHK cells, SP2/0, NSO cells, carrot cells, and derivatives thereof. Insect cells include cells of Drosophila melanogaster origin. These cells can be genetically engineered to render the cells capable of making immunoglobulins that have particular or predominantly particular N-glycans. For example, U.S. Pat. No. 6,949,372 discloses methods for making glycoproteins in insect cells that are sialylated. Yamane-Ohnuki et al. Biotechnol. Bioeng. 87: 614-622 (2004), Kanda et al., Biotechnol. Bioeng. 94: 680-688 (2006), Kanda et al., Glycobiol. 17: 104-118 (2006), and U.S. Pub. Application Nos. 2005/0216958 and 2007/0020260 (the disclosures of which are incorporated herein by reference) disclose mammalian cells that are capable of producing immunoglobulins in which the N-glycans thereon lack fucose or have reduced fucose. U.S. Published Patent Application No. 2005/0074843 (the disclosure of which is incorporated herein by reference) discloses making antibodies in mammalian cells that have bisected N-glycans.

The regulatable promoters selected for regulating expression of the expression cassettes in mammalian, insect, or plant cells should be selected for functionality in the cell-type chosen. Examples of suitable regulatable promoters include but are not limited to the tetracycline-regulatable promoters (See for example, Berens & Hillen, Eur. J. Biochem. 270: 3109-3121 (2003)), RU 486-inducible promoters, ecdysone-inducible promoters, and kanamycin-regulatable systems. These promoters can replace the promoters exemplified in the expression cassettes described in the examples. The capture moiety can be fused to a cell surface anchoring protein suitable for use in the cell-type chosen. Cell surface anchoring proteins including GPI proteins are well known for mammalian, insect, and plant cells. GPI-anchored fusion proteins has been described by Kennard et al., Methods Biotechnol. Vo. 8: Animal Cell Biotechnology (Ed. Jenkins. Human Press, Inc., Totowa, N.J.) pp. 187-200 (1999). The genome targeting sequences for integrating the expression cassettes into the host cell genome for making stable recombinants can replace the genome targeting and integration sequences exemplified in the examples. Transfection methods for making stable and transiently transfected mammalian, insect, and plant host cells are well known in the art. Once the transfected host cells have been constructed as disclosed herein, the cells can be screened for expression of the immunoglobulin of interest and selected as disclosed herein.

Therefore, in a further aspect of the above, provided is a method for producing an N-glycosylated insulin or insulin analogue in a mammalian, plant, or insect host cell, comprising providing a mammalian or insect host cell that includes a nucleic acid molecule encoding a heterologous single-subunit oligosaccharyltransferase (e.g., Leishmania major STT3 protein) and a nucleic acid molecule encoding the insulin or insulin analogue having at least one N-glycosylation site; and culturing the host cell under conditions for expressing the insulin or insulin analogue to produce the N-glycosylated insulin analogue. In further aspects, the host cell is genetically engineered to produce glycoproteins with predominantly a particular N-glycan species, for example, produce glycoproteins that have human-like N-glycans or N-glycans not normally endogenous to the host cell.

In a further aspect of the above, provided is a method for producing an insulin or insulin analogue wherein the N-glycosylation site occupancy of the insulin or insulin analogue is greater than 83% in a mammalian or insect host cell, comprising providing a mammalian or insect host cell that includes a nucleic acid molecule encoding a heterologous single-subunit oligosaccharyltransferase (e.g., Leishmania major STT3 protein) and a nucleic acid molecule encoding the insulin or insulin analogue having at least one N-glycosylation site; and culturing the host cell under conditions for expressing the insulin or insulin analogue having at least one N-glycosylation site to produce the insulin or insulin analogue wherein the N-glycosylation site occupancy of the insulin or insulin analogue is greater than 83%. In further aspects, the host cell is genetically engineered to produce glycoproteins with human-like N-glycans or N-glycans not normally endogenous to the host cell.

In a further embodiment of the above methods, the endogenous host cell genes encoding the proteins comprising the oligosaccharyltransferase (OTase) complex are expressed.

In particular embodiments of the above methods, the N-glycosylation site occupancy is at least 94%. In further still embodiments, the N-glycosylation site occupancy is at least 99%.

Further provided is a mammalian or insect host cell, comprising a first nucleic acid molecule encoding a heterologous single-subunit oligosaccharyltransferase (e.g., the Leishmania major STT3D protein); and a second nucleic acid molecule encoding an insulin or insulin analogue having at least one N-glycosylation site; and wherein the endogenous host cell genes encoding the proteins comprising the endogenous host cell oligosaccharyltransferase (OTase) complex are expressed.

In particular embodiments, the higher eukaryote cell, tissue, or organism can also be from the plant kingdom, for example, wheat, rice, corn, carrot, tobacco, and the like. Alternatively, bryophyte cells can be selected, for example from species of the genera Physcomitrella, Funaria, Sphagnum, Ceratodon, Marchantia, and Sphaerocarpos. Exemplary of plant cells is the bryophyte cell of Physcomitrella patens, which has been disclosed in WO 2004/057002 and WO2008/006554 (the disclosures of which are all incorporated herein by reference). Expression systems using plant cells can further manipulated to have altered glycosylation pathways to enable the cells to produce glycoproteins that have predominantly particular N-glycans. For example, the cells can be genetically engineered to have a dysfunctional or no core fucosyltransferase and/or a dysfunctional or no xylosyltransferase, and/or a dysfunctional or no β1,4-galactosyltransferase. Alternatively, the galactose, fucose and/or xylose can be removed from the glycoprotein by treatment with enzymes removing the residues. Any enzyme resulting in the release of galactose, fucose and/or xylose residues from N-glycans which are known in the art can be used, for example α-galactosidase, β-xylosidase, and α-fucosidase. Alternatively, an expression system can be used which synthesizes modified N-glycans which can not be used as substrates by 1,3-fucosyltransferase and/or 1,2-xylosyltransferase, and/or 1,4-galactosyltransferase. Methods for modifying glycosylation pathways in plant cells are disclosed in U.S. Pat. Nos. 7,449,308, 6,998,267 and 7,388,081 (the disclosures of which are incorporated herein by reference) which disclose methods for genetically engineering plants to make recombinant glycoproteins that have human-like N-glycans. WO 2008006554 (the disclosure of which is incorporated herein by reference) discloses methods for making glycoproteins such as antibodies in plants genetically engineered to make glycoproteins without xylose or fucose. WO 2007006570 (the disclosure of which is incorporated herein by reference) discloses methods for genetically engineering bryophytes, ciliates, algae, and yeast to make glycoproteins that have animal or human-like glycosylation patterns.

Therefore, in a further aspect of the above, provided is a method for producing an N-glycosylated insulin or insulin analogue with predominantly a particular N-glycan species in a plant host cell, comprising providing a plant host cell that is genetically engineered to produce glycoproteins that have mammalian- or human-like N-glycans and includes a nucleic acid molecule encoding a heterologous single-subunit oligosaccharyltransferase (e.g., the Leishmania major STT3D protein) and a nucleic acid molecule encoding the insulin or insulin analogue having at least N-glycosylation site; and culturing the host cell under conditions for expressing the insulin or insulin analogue to produce the N-glycosylated insulin or insulin analogue.

In a further aspect of the above, provided is a method for producing an insulin or insulin analogue with a predominant N-glycan species wherein the N-glycosylation site occupancy of the insulin or insulin analogue is greater than 83% in a plant host cell, comprising providing a plant host cell that is genetically engineered to produce glycoproteins that have predominantly a particular N-glycan species and includes a nucleic acid molecule encoding a heterologous single-subunit oligosaccharyltransferase (e.g., the Leishmania major STT3D protein) and a nucleic acid molecule encoding the insulin or insulin analogue having at least one N-glycosylation site; and culturing the host cell under conditions for expressing the insulin or insulin analogue to produce the N-glycosylated insulin or insulin analogue wherein the N-glycosylation site occupancy is greater than 83%.

In a further embodiment of the above methods, the endogenous host cell genes encoding the proteins comprising the endogenous host cell oligosaccharyltransferase (OTase) complex are expressed.

In particular embodiments of the above methods, the N-glycosylation site occupancy is at least 94%. In further still embodiments, the N-glycosylation site occupancy is at least 99%.

Further provided is a plant host cell, comprising a first nucleic acid molecule encoding a heterologous single-subunit oligosaccharyltransferase (e.g., the Leishmania major STT3D protein); and a second nucleic acid molecule encoding an insulin or insulin analogue having at least one N-glycosylation site; and wherein the endogenous host cell genes encoding the proteins comprising the endogenous host cell oligosaccharyltransferase (OTase) complex are expressed.

### VI. Sustained Release Formulations

In certain embodiments it may be advantageous to administer an in vivo N-glycosylated or in vitro glycosylated insulin or insulin analogue in a sustained fashion (i.e., in a form that exhibits an absorption profile that is more sustained than soluble recombinant human insulin). This will provide a sustained level of glycosylated insulin that can respond to fluctuations in glucose on a timescale that it more closely related to the typical glucose fluctuation timescale (i.e., hours rather than minutes). In certain embodiments, the sustained release formulation may exhibit a zero-order release of the glycosylated insulin when administered to a mammal under non-hyperglycemic conditions (i.e., fasted conditions). It will be appreciated that any formulation that provides a sustained absorption profile may be used. In certain embodiments this may be achieved by combining the glycosylated insulin with other ingredients that slow its release properties into systemic circulation. For example, PZI (protamine zinc insulin) formulations may be used for this purpose. In some cases, the zinc content is in the range of about 0.05 to about 0.5 mg zinc/mg glycosylated insulin.

Thus, in certain embodiments, a formulation of the present disclosure includes from about 0.05 to about 10 mg protamine/mg glycosylated insulin or insulin analogue. For example, from about 0.2 to about 10 mg protamine/mg glycosylated insulin or insulin analogue, e.g., about 1 to about 5 mg protamine/mg glycosylated insulin or insulin analogue.

In certain embodiments, a formulation of the present disclosure includes from about 0.006 to about 0.5 mg zinc/mg glycosylated insulin or insulin analogue. For example, from about 0.05 to about 0.5 mg zinc/mg glycosylated insulin or insulin analogue, e.g., about 0.1 to about 0.25 mg zinc/mg glycosylated insulin or insulin analogue.

In certain embodiments, a formulation of the present disclosure includes protamine and zinc in a ratio (w/w) in the range of about 100:1 to about 5:1, for example, from about 50:1 to 20 about 5:1, e.g., about 40:1 to about 10:1. In certain embodiments, a PZI formulation of the present disclosure includes protamine and zinc in a ratio (w/w) in the range of about 20:1 to about 5:1, for example, about 20:1 to about 10:1, about 20:1 to about 15:1, about 15:1 to about 5:1, about 10:1 to about 5:1, about 10:1 to about 15:1.

In certain embodiments a formulation of the present disclosure includes an antimicrobial preservative (e.g., m-cresol, phenol, methylparaben, or propylparaben). In certain embodiments the antimicrobial preservative is m-cresol. For example, in certain embodiments, a formulation may include from about 0.1 to about 1.0% v/v m-cresol. For example, from about 0.1 to about 0.5% v/v m-cresol, e.g., about 0.15 to about 0.35% v/v m-cresol.

In certain embodiments a formulation of the present disclosure includes a polyol as isotonic agent (e.g., mannitol, propylene glycol or glycerol). In certain embodiments the isotonic agent is glycerol. In certain embodiments, the isotonic agent is a salt, e.g., NaCl. For example, a formulation may comprise from about 0.05 to about 0.5 M NaCl, e.g., from about 0.05 to about 0.25 M NaCl or from about 0.1 to about 0.2 M NaCl.

In certain embodiments a formulation of the present disclosure includes an amount of non-glycosylated insulin or insulin analogue. In certain embodiments, a formulation includes a molar ratio of glycosylated insulin analogue to non-glycosylated insulin or insulin analogue in the range of about 100:1 to 1:1, e.g., about 50:1 to 2:1 or about 25:1 to 2:1.

The present disclosure also encompasses the use of standard sustained (also called extended) release formulations that are well known in the art of small molecule formulation (e.g., see Remington's Pharmaceutical Sciences, 19th ed., Mack Publishing Co., Easton, Pa., 1995).

The present disclosure also encompasses the use of devices that rely on pumps or hindered diffusion to deliver a glycosylated insulin analogue on a gradual basis. In certain embodiments, a long acting formulation may (additionally or alternatively) be provided by modifying the insulin to be long-lasting. For example, the insulin analogue may be insulin glargine or insulin detemir. Insulin glargine is an exemplary long acting insulin analogue in which Asn-A21 has been replaced by glycine, and two arginines have been added to the C-terminus of the B-chain. The effect of these changes is to shift the isoelectric point, producing a solution that is completely soluble at pH 4. Insulin detemir is another long acting insulin analogue in which Thr-B30 has been deleted, and a C14 fatty acid chain has been attached to Lys-B29.

The following examples are intended to promote a further understanding of the present invention.

### Example 1

This example illustrates the construction of plasmid expression vectors encoding human insulin analogues comprising a substitution of the proline residue at position 28 of the B-chain with an asparagine residue to produce an N-glycosylation site having the tri-amino acid sequence Asn Xaa (Ser/Thr) wherein Xaa is any amino acid except Pro. These expression vectors have been designed for protein expression in Pichia pastoris; however, the nucleic acid molecules encoding the recited insulin analogue A- and B-chains can be incorporated into expression vectors designed for protein expression in other host cells capable of producing N-glycosylated glycoproteins, for example, mammalian cells and fungal, plant, insect, or bacterial cells, including host cells genetically modified to produce glycoproteins having human-like N-glycans.

The expression vectors disclosed below encode a pre-proinsulin analogue precursor molecule. During expression of the vector encoding the pre-proinsulin analogue precursor in the yeast host cell, the pre-proinsulin analogue precursor is transported to the secretory pathway where the signal peptide is removed and the molecule is processed into an N-glycosylated proinsulin analogue precursor that is folded into a structure held together by disulfide bonds that has the same configuration as that for native human insulin. The N-glycosylated proinsulin analogue precursor is then transported through the secretory pathway where the N-glycans on the N-glycosylated proinsulin analogue precursor are modified. The N-glycosylated proinsulin analogue precursor is then directed to vesicles where the propeptide is removed to form an N-glycosylated insulin analogue precursor molecule that is then secreted from the host cell where it can be further processed in vitro using trypsin or endoproteinase Lys-C digestion to produce an N-glycosylated insulin analogue heterodimer.

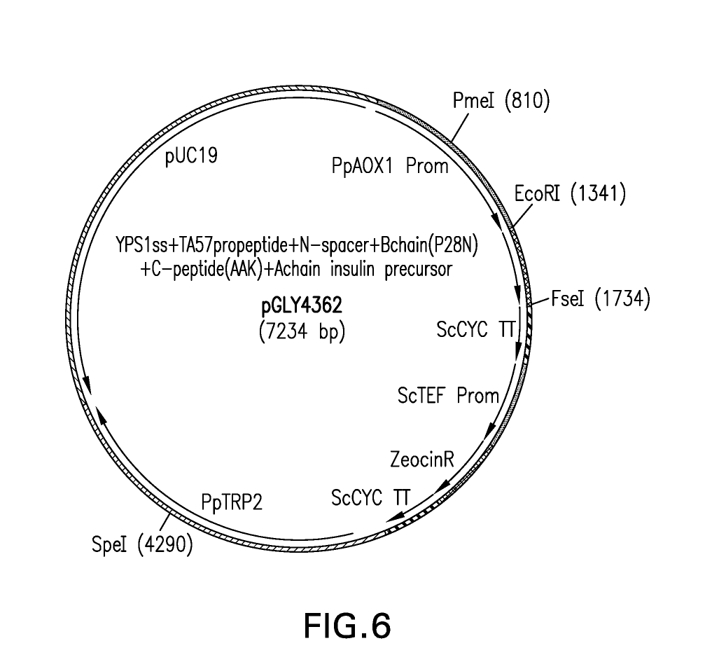

Plasmid pGLY4362 (FIG. 6) is a roll-in integration plasmid that targets the TRP2 locus or AOX1 locus and includes an expression cassette encoding a pre-proinsulin analogue precursor comprising a Yps1ss peptide (SEQ ID NO:20) fused to a TA57 propeptide (SEQ ID NO:21) fused to an N-terminal spacer (SEQ ID NO:22) fused to the human insulin B-chain with a P28N substitution (SEQ ID NO:26) fused to a C-peptide consisting of the amino acid sequence AAK (SEQ ID NO:31) fused to the human insulin A-chain (SEQ ID NO:33). The pre-proinsulin analogue precursor has the amino acid sequence shown in SEQ ID NO:6 and is encoded by the nucleotide sequence shown in SEQ ID NO:5. The proinsulin with N-terminal spacer has the amino acid sequence shown in SEQ ID NO:36 and the proinsulin analogue without N-terminal spacer has the amino acid sequence shown in SEQ ID NO:37. The expression cassette comprises a nucleic acid molecule encoding the fusion protein (SEQ ID NO:5) operably linked at the 5′ end to a nucleic acid molecule that has the inducible P. pastoris AOX1 promoter sequence (SEQ ID NO:118) and at the 3′ end to a nucleic acid molecule that has the Saccharomyces cerevisiae CYC transcription termination sequence (SEQ ID NO:58). For selecting transformants, the plasmid comprises an expression cassette encoding the Zeocin ORF in which the nucleic acid molecule encoding the ORF (SEQ ID NO:122) is operably linked at the 5′ end to a nucleic acid molecule having the S. cerevisiae TEF promoter sequence (SEQ ID NO:123) and at the 3′ end to a nucleic acid molecule having the S. cerevisiae CYC transcription termination sequence (SEQ ID NO:58). The plasmid further includes a nucleic acid molecule for targeting the TRP2 locus.

The Yps1ss peptide is a synthetic leader or signal peptide disclosed in U.S. Pat. Nos. 5,639,642 and 5,726,038, and which are hereby incorporated herein by reference. The TA57 propeptide and N-terminal spacer have been described by Kjeldsen et al., Gene 170:107-112 (1996) and in U.S. Pat. Nos. 6,777,207, and 6,214,547, and which are hereby incorporated herein in by reference. Other synthetic propeptides are disclosed in U.S. Pat. Nos. 5,395,922, 5,795,746, and 5,162,498; and WO 9832867, and which are hereby incorporated herein in by reference.

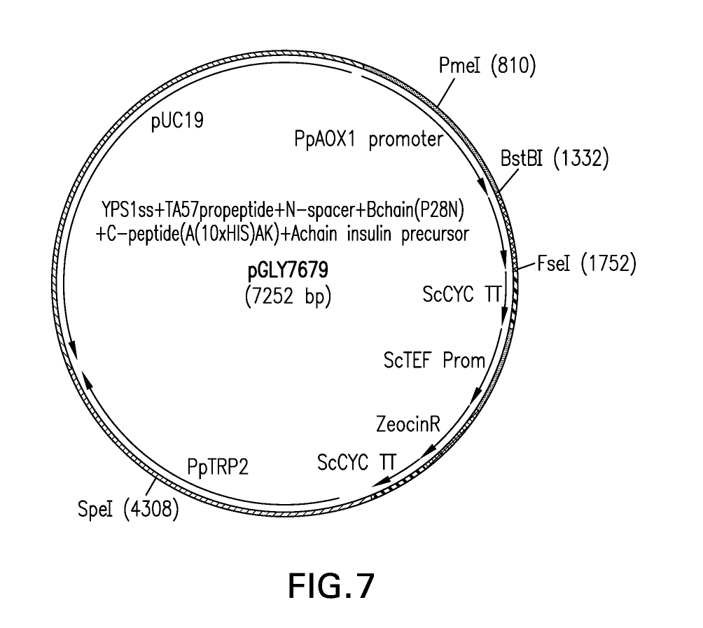

Plasmid pGLY7679 (FIG. 7) is similar to pGLY4362 except that the expression cassette encodes a pre-proinsulin analogue precursor comprising a Yps1ss peptide (SEQ ID NO:20) fused to a TA57 propeptide (SEQ ID NO:21) fused to an N-terminal spacer peptide (SEQ ID NO:22) fused to the human insulin B-chain with a P28N substitution (SEQ ID NO:26) fused to a C-peptide consisting of the amino acid sequence A(10×HIS)AK (SEQ ID NO:32) fused to the human insulin A-chain (SEQ ID NO:33). The pre-proinsulin analogue precursor has the amino acid sequence shown in SEQ ID NO:8 and is encoded by the nucleotide sequence shown in SEQ ID NO:7. The proinsulin with N-terminal spacer has the amino acid sequence shown in SEQ ID NO:36 and the proinsulin analogue without N-terminal spacer has the amino acid sequence shown in SEQ ID NO:37.

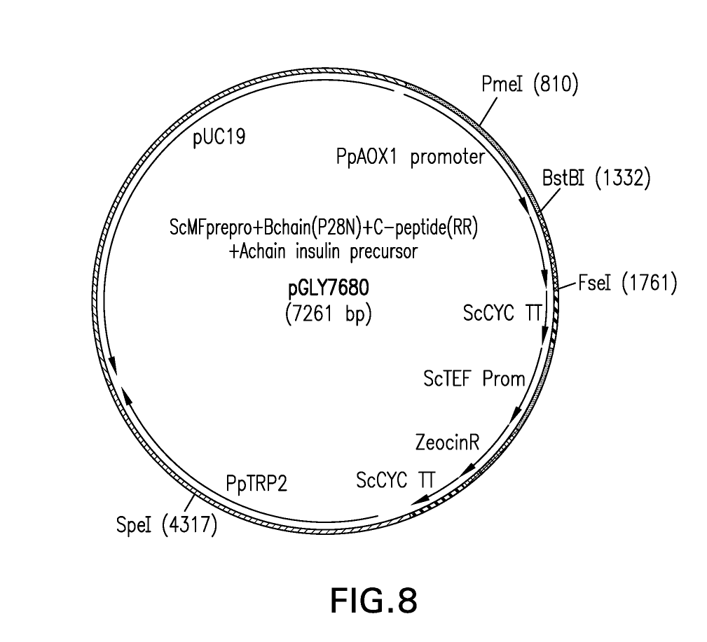

Plasmid pGLY7680 (FIG. 8) is similar to pGLY4362 except that the expression cassette encodes a pre-proinsulin analogue precursor comprising a S. cerevisiae alpha mating factor signal sequence and propeptide (SEQ ID NO:19) fused to the human insulin B-chain with a P28N substitution (SEQ ID NO:26) fused to a C-peptide consisting of the amino acid sequence RR fused to the human insulin A-chain (SEQ ID NO:33). The pre-proinsulin analogue precursor has the amino acid sequence shown in SEQ ID NO:10 and is encoded by the nucleotide sequence shown in SEQ ID NO:9. The S. cerevisiae alpha mating factor signal sequence has been described in U.S. Pat. Nos. 6,777,207, 4,546,082 and 4,870,008, and which are incorporated herein by reference. The proinsulin analogue has the amino acid sequence shown in SEQ ID NO:37.

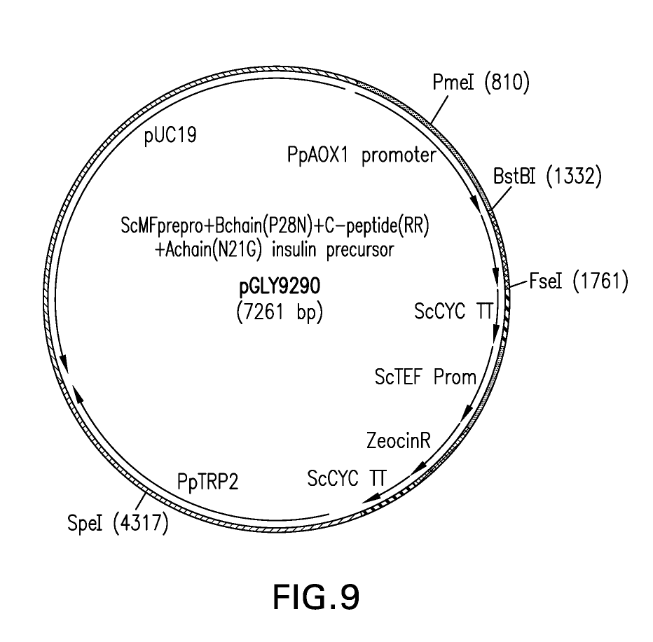

Plasmid pGLY9290 (FIG. 9) is similar to pGLY4362 except that the expression cassette encodes a pre-proinsulin analogue precursor comprising a S. cerevisiae alpha mating factor signal sequence and propeptide (SEQ ID NO:19) fused to the human insulin B-chain with a P28N substitution (SEQ ID NO:26) fused to a C-peptide consisting of the amino acid sequence RR fused to the human insulin A-chain with an N21G substitution (SEQ ID NO:34). The pre-proinsulin analogue precursor has the amino acid sequence shown in SEQ ID NO:12 and is encoded by the nucleotide sequence shown in SEQ ID NO:11. Processing of the pre-proinsulin analogue precursor when it enters the secretory pathway produces a proinsulin analogue having the amino acid sequence shown in SEQ ID NO:38.

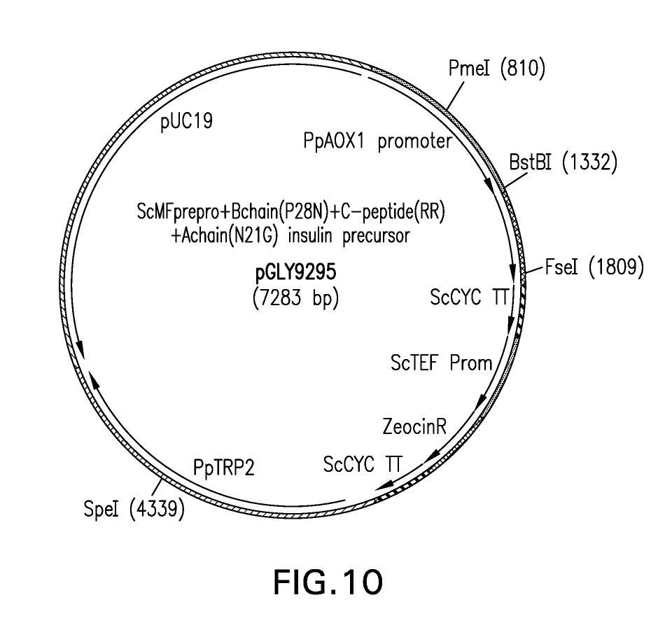

Plasmid pGLY9295 (FIG. 10) is similar to pGLY4362 except that the expression cassette encodes a pre-proinsulin analogue precursor comprising a S. cerevisiae alpha mating factor signal sequence and propeptide (SEQ ID NO:19) fused to an N-terminal HIS spacer peptide (SEQ ID NO:23) fused to the human insulin B-chain with a P28N substitution (SEQ ID NO:26) fused to a C-peptide consisting of the amino acid sequence RR fused to the human insulin A-chain with an N21G substitution (SEQ ID NO:34). The pre-proinsulin analogue precursor has the amino acid sequence shown in SEQ ID NO:14 and is encoded by the nucleotide sequence shown in SEQ ID NO:13. In addition, the expression cassette comprises the P. pastoris AOX1 transcription termination sequence. The proinsulin with N-terminal spacer has the amino acid sequence shown in SEQ ID NO:41 and the proinsulin analogue without N-terminal spacer has the amino acid sequence shown in SEQ ID NO:38.

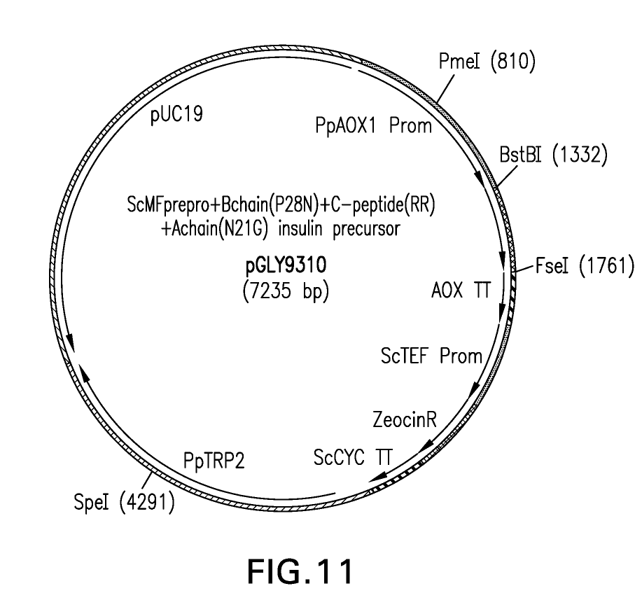

Plasmid pGLY9310 (FIG. 11) is similar to pGLY4362 except that the expression cassette encodes a pre-proinsulin analogue precursor comprising a S. cerevisiae alpha mating factor signal sequence and propeptide (SEQ ID NO:19) fused to the human insulin B-chain with a P28N substitution (SEQ ID NO:26) fused to a C-peptide consisting of the amino acid sequence RR fused to the human insulin A-chain with an N21G substitution (SEQ ID NO:34). The pre-proinsulin analogue precursor has the amino acid sequence shown in SEQ ID NO:12 and is encoded by the nucleotide sequence shown in SEQ ID NO:11. In addition, the expression cassette comprises the P. pastoris AOX1 transcription termination sequence. Processing of the pre-proinsulin analogue precursor when it enters the secretory pathway produces a proinsulin analogue having the amino acid sequence shown in SEQ ID NO:28.

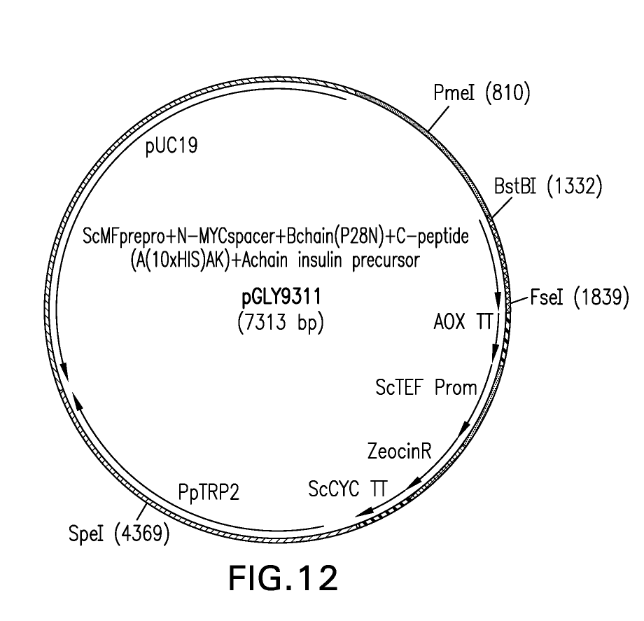

Plasmid pGLY9311 (FIG. 12) is similar to pGLY4362 except that the expression cassette encodes a pre-proinsulin analogue precursor comprising a S. cerevisiae alpha mating factor signal sequence and propeptide (SEQ ID NO:19) fused to an N-terminal MYC spacer peptide (SEQ ID NO:24) fused to the human insulin B-chain with a P28N substitution (SEQ ID NO:26) fused to a C-peptide consisting of the amino acid sequence A(10×HIS)AK (SEQ ID NO:32) fused to the human insulin A-chain (SEQ ID NO:33). The pre-proinsulin analogue precursor has the amino acid sequence shown in SEQ ID NO:16 and is encoded by the nucleotide sequence shown in SEQ ID NO:15. The proinsulin with N-terminal spacer has the amino acid sequence shown in SEQ ID NO:40. In addition, the expression cassette comprises the P. pastoris AOX1 transcription termination sequence.

Plasmid pGLY9312 is similar to pGLY9311 except that nucleotide sequence encoding the expression cassette has been optimized for Pichia pastoris codon usage utilizing an alternative codon optimization algorithm (SEQ ID NO:17). Table 1 summarizes the elements of the above expression cassettes.

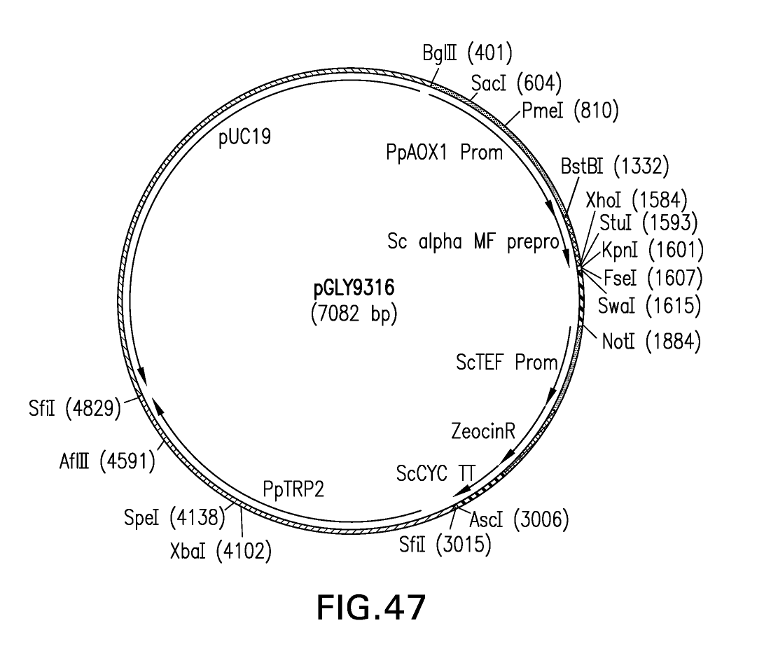

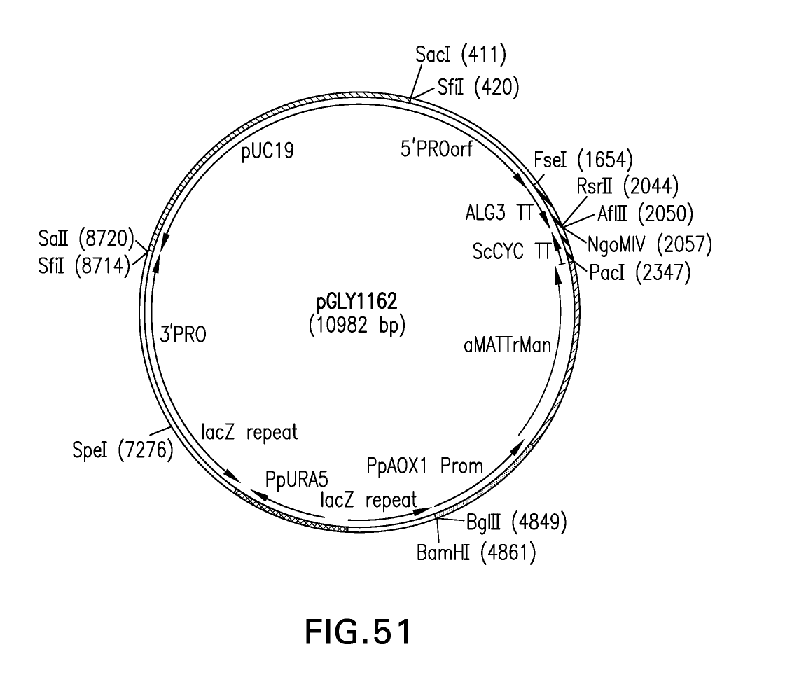

Plasmid pGLY9316 (FIG. 47) is an empty expression plasmid that was used to generate insulin expression plasmids pGLY11074, pGLY11084, pGLY11085, pGLY11087, pGLY11088, pGLY11098, pGLY11099 (FIG. 51), pGLY11101, pGLY11164, pGLY11464, and pGLY11465 that are listed in Table 1. Plasmid pGLY9316 is similar to pGLY4362 except that the expression cassette contains the S. cerevisiae alpha mating factor signal sequence and propeptide (SEQ ID NO:148) but not insulin precursor sequence. Descendent insulin precursor expression plasmids, as listed in Table 1, were constructed by cloning the insulin precursor DNA that encodes an N-terminal spacer peptide (SEQ ID NO:149) fused to the human insulin sequence variants using MlyI and FseI. The nucleic acid molecules encoding the insulin variants are SEQ ID NO:126 encoding SEQ ID NO:127 (pGLY11074), SEQ ID NO:128 encoding SEQ ID NO:129 (pGLY11084), SEQ ID NO: 130 encoding SEQ ID NO:131 (pGLY11085), SEQ ID NO:132 encoding SEQ ID NO:133 (pGLY11087), SEQ ID NO:134 encoding SEQ ID NO:135 (pGLY11088), SEQ ID NO:136 encoding SEQ ID NO:137 (pGLY11098), SEQ ID NO:138 encoding SEQ ID NO:139 (pGLY11099), SEQ ID NO:140 encoding SEQ ID NO:141 (pGLY11101), SEQ ID NO:142 encoding SEQ ID NO:143 (pGLY11164), SEQ ID NO:144 encoding SEQ ID NO:145 (pGLY11464), and SEQ ID NO:146 encoding SEQ ID NO:147 (pGLY11465). The proinsulin analogue precursor sequences produced by these vectors are listed in Table 1. In addition, the expression cassette comprises the P. pastoris AOX1 transcription termination sequence.

TABLE 1

| | Modifications of the encoded | No. of | Proinsulin analogue |
|---|---|---|---|
| Expression | Proinsulin Analogue Precursor with | Glycosylation | precursor |
| vector | “AAK” C-peptide | sites | SEQ ID NO: |
| pGLY11074 | B:des(B30) | 0 | 150 |
| pGLY11084 | B:NTT(−2) des(B30) | 1 | 151 |
| pGLY11085 | B:NGT(−2) des(B30) | 1 | 152 |
| pGLY11087 | A:NTT(−2) des(B30) | 1 | 153 |
| pGLY11088 | B:P28N | 1 | 154 |
| pGLY11098 | B:NTT(−2) + B:P28N | 2 | 155 |
| pGLY11099 | B:NGT(−2) + B:P28N | 2 | 156 |
| pGLY11101 | B:P28N + A:NTT(−2) | 2 | 157 |
| pGLY11164 | B:P28N des(B30) | 0 | 158 |
| pGLY11464 | B:NGT(−2) des(B30) + A:NGT(−2) | 2 | 159 |
| pGLY11465 | B:NGT(−2) + B:P28N + A:NGT(−2) | 3 | 160 |

The designation des(B30) indicates that the amino acid sequence lacks the amino acid threonine at position B30. Unless otherwise indicated, the A chain includes amino acids 1-21 of the native human A-chain.

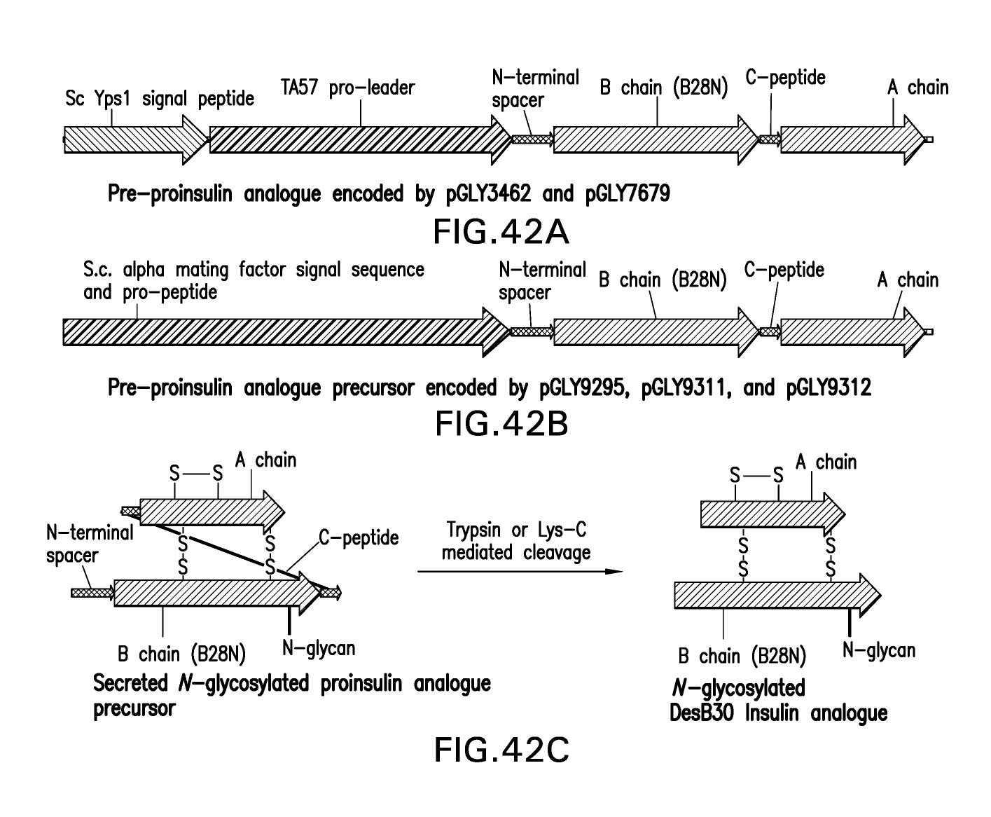

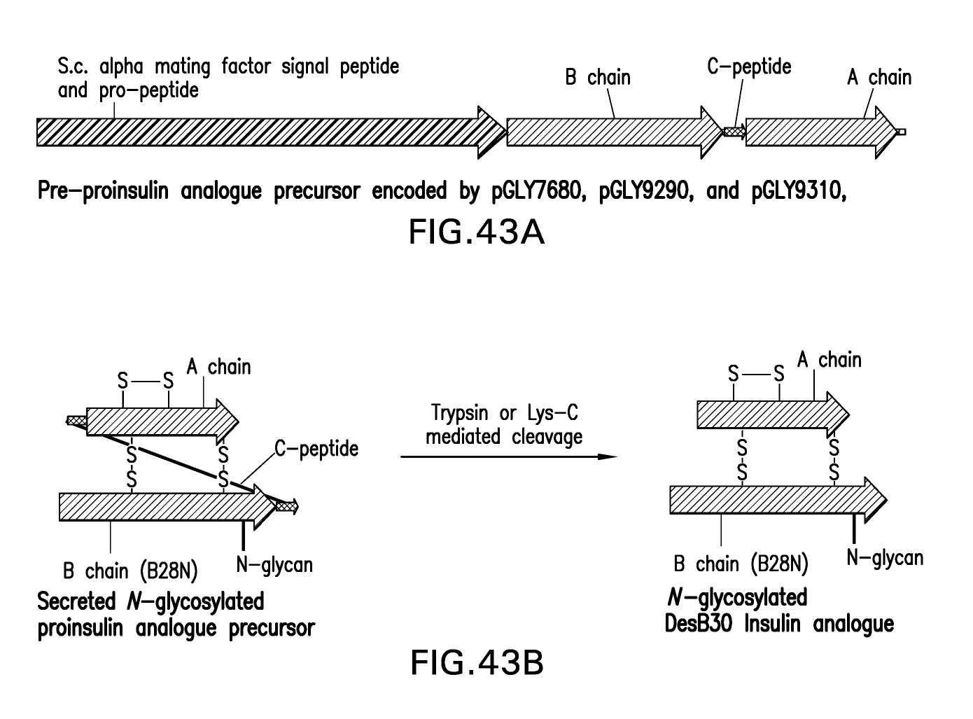

The expression vector containing the expression cassette encoding the pre-proinsulin analogue precursor is transformed into a yeast host cell capable of making N-linked glycoproteins. As illustrated in FIG. 42A, FIG. 42B, and FIG. 42C and FIG. 43A and FIG. 43B, the pre-proinsulin analogue precursor is expressed from the expression cassette integrated into the host cell genome. The pre-proinsulin analogue precursor targets the secretory pathway where it is folded with disulfide linkages and N-glycosylated. The N-glycosylated proinsulin analogue precursor is further processed in the Golgi apparatus and then transported to vesicles where the propeptide is removed and the N-glycosylated pre-proinsulin analogue precursor is secreted from the host cell into the culture medium where it may be purified and further processed in vitro (ex-cellular) to remove the C-peptide and the N-terminal peptide to provide an N-glycosylated insulin analogue heterodimer that comprises an N-linked N-glycan. The particular N-glycosylated insulin analogues that are produced from the above precursors following in vitro processing with trypsin or endoproteinase Lys-C lack the B30 Tyrosine residue, thus the N-glycosylated insulin analogues are desB30 analogues. However, as known in the art, desB30 insulin analogues have an activity at the insulin receptor that is not substantially different from that of native insulin.

### Example 2

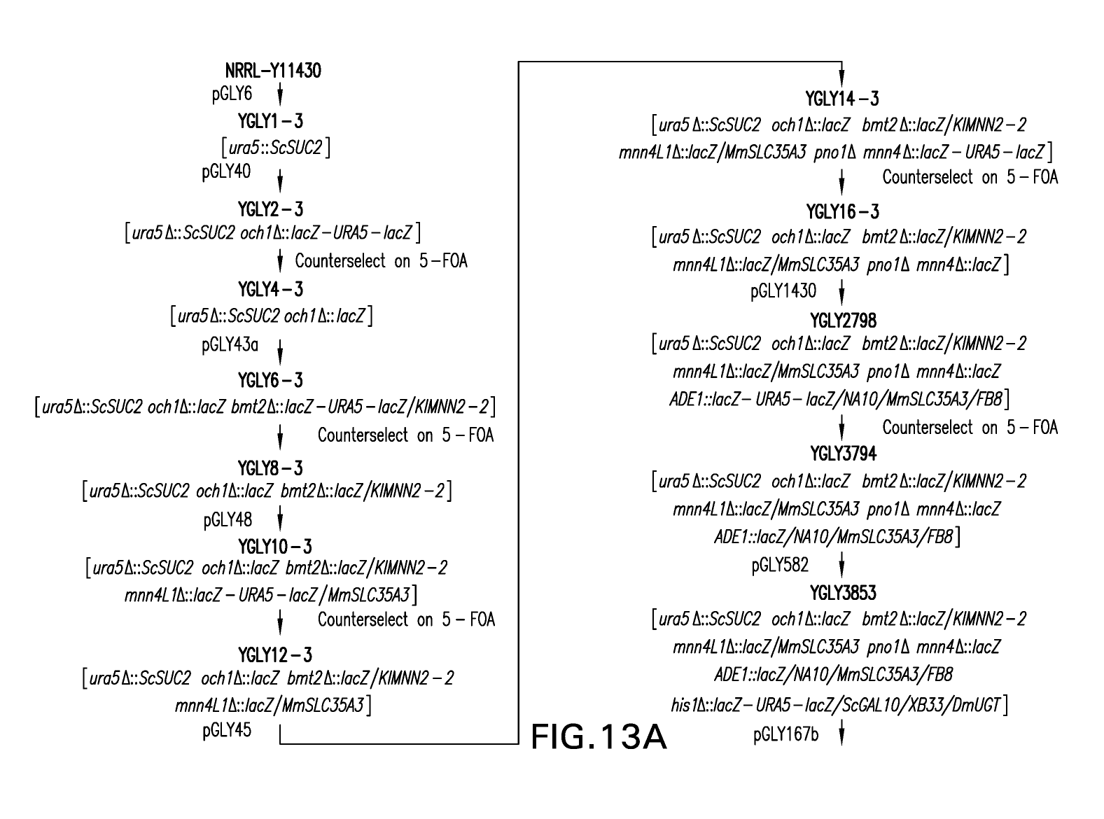

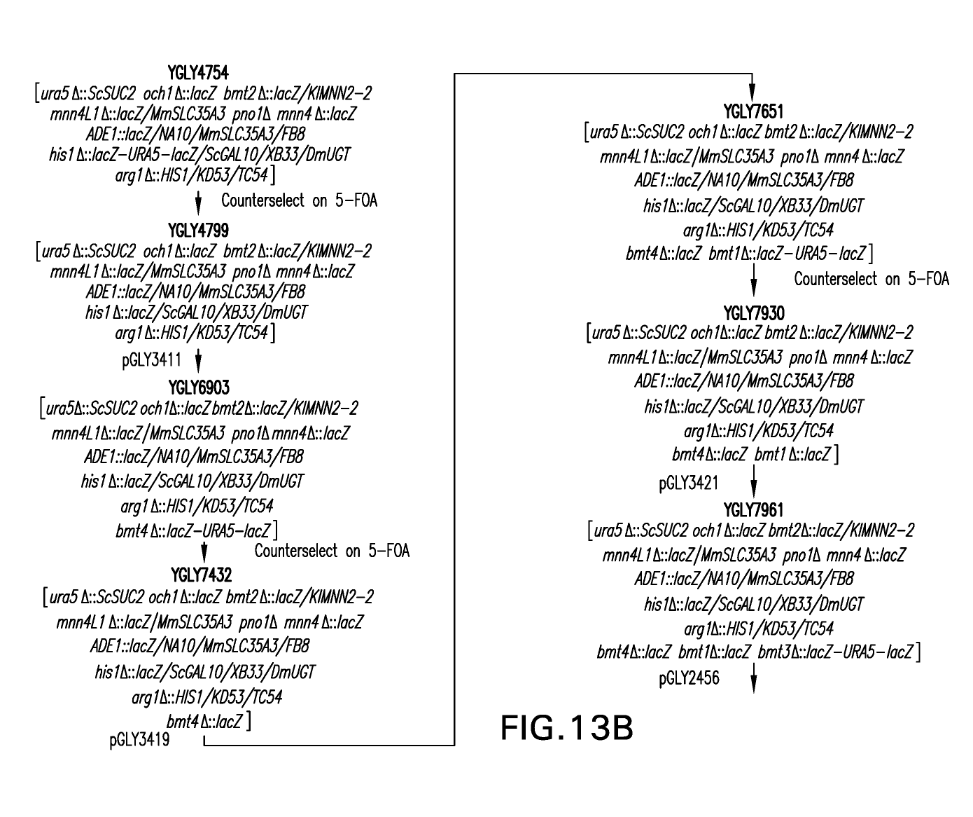

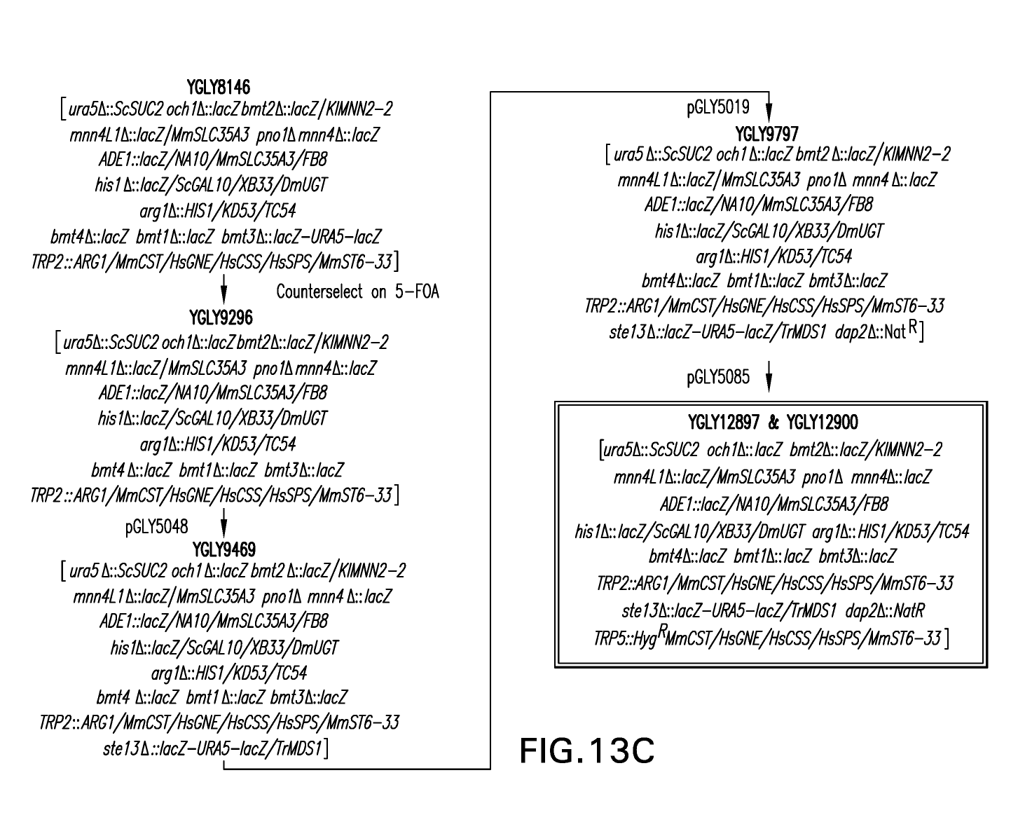

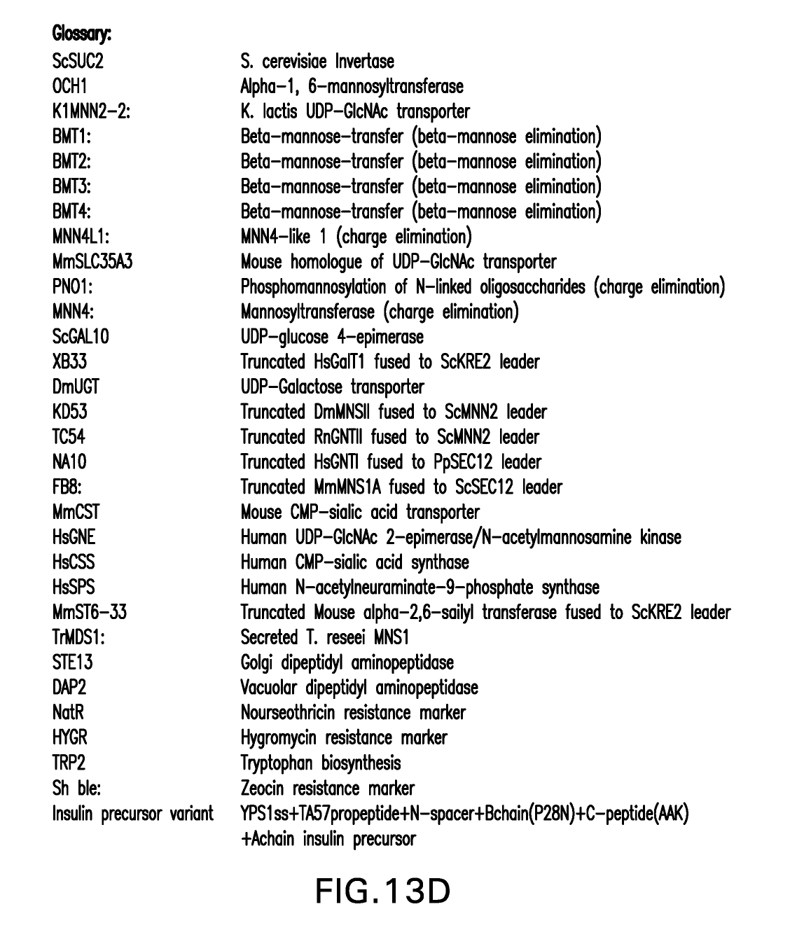

A Pichia pastoris strain capable of producing sialylated N-glycans was constructed as follows. Construction of the strain is illustrated schematically in FIG. 13A-13D. Briefly, the strain was constructed as follows.

The strain YGLY8316 was constructed from wild-type Pichia pastoris strain NRRL-Y 11430 using methods described earlier (See for example, U.S. Pat. No. 7,449,308; U.S. Pat. No. 7,479,389; U.S. Published Application No. 20090124000; Published PCT Application No. WO2009085135; Nett and Gerngross, Yeast 20:1279 (2003); Choi et al., Proc. Natl. Acad. Sci. USA 100:5022 (2003); Hamilton et al., Science 301:1244 (2003)). All plasmids were made in a pUC19 plasmid using standard molecular biology procedures. For nucleotide sequences that were optimized for expression in P. pastoris, the native nucleotide sequences were analyzed by the GENEOPTIMIZER software (GeneArt, Regensburg, Germany) and the results used to generate nucleotide sequences in which the codons were optimized for P. pastoris expression.

Yeast strains were transformed by electroporation (using standard techniques as recommended by the manufacturer of the electroporator BioRad). In general, yeast transformations were as follows. P. pastoris strains were grown in 50 mL YPD media (yeast extract (1%), peptone (2%), dextrose (2%)) overnight to an optical density (“OD”) of between about 0.2 to 6. After incubation on ice for 30 minutes, cells were pelleted by centrifugation at 2500-3000 rpm for 5 minutes. Media was removed and the cells washed three times with ice cold sterile 1M sorbitol before resuspension in 0.5 ml ice cold sterile 1M sorbitol. Ten μL DNA (5-20 μs) and 100 μL cell suspension was combined in an electroporation cuvette and incubated for 5 minutes on ice. Electroporation was in a Bio-Rad GenePulser Xcell following the preset Pichia pastoris protocol (2 kV, 25 μF, 200Ω), immediately followed by the addition of 1 mL YPDS recovery media (YPD media plus 1 M sorbitol). The transformed cells were allowed to recover for four hours to overnight at room temperature (26° C.) before plating the cells on selective media.

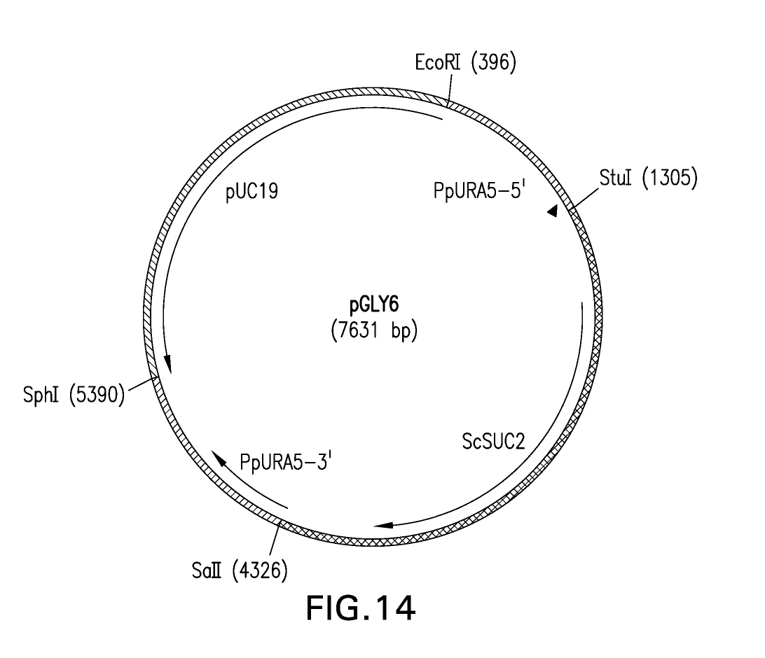

Plasmid pGLY6 (FIG. 14) is an integration vector that targets the URA5 locus. It contains a nucleic acid molecule comprising the S. cerevisiae invertase gene or transcription unit (ScSUC2; SEQ ID NO:46) flanked on one side by a nucleic acid molecule comprising a nucleotide sequence from the 5′ region of the P. pastoris URA5 gene (SEQ ID NO:47) and on the other side by a nucleic acid molecule comprising the nucleotide sequence from the 3′ region of the P. pastoris URA5 gene (SEQ ID NO:48). Plasmid pGLY6 was linearized and the linearized plasmid transformed into wild-type strain NRRL-Y 11430 to produce a number of strains in which the ScSUC2 gene was inserted into the URA5 locus by double-crossover homologous recombination. Strain YGLY1-3 was selected from the strains produced and is auxotrophic for uracil.

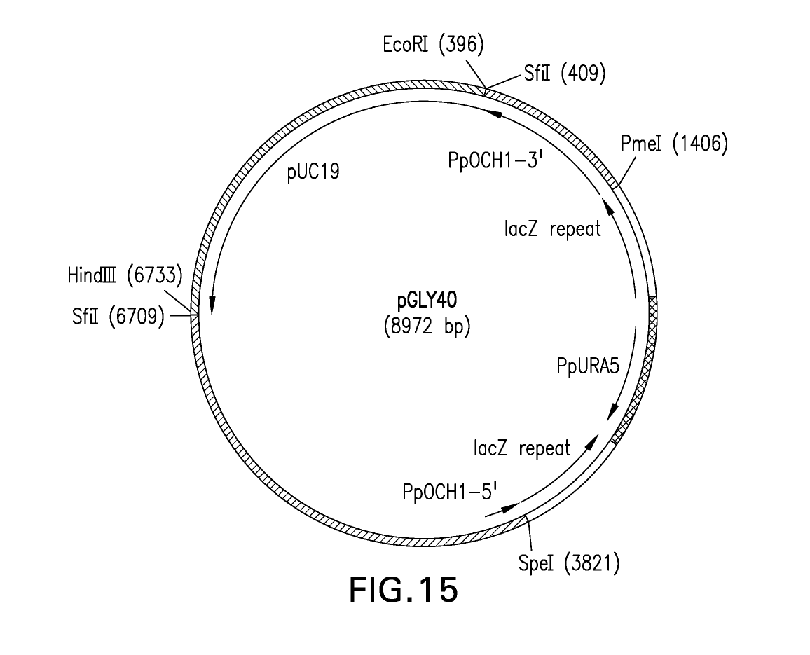

Plasmid pGLY40 (FIG. 15) is an integration vector that targets the OCH1 locus and contains a nucleic acid molecule comprising the P. pastoris URA5 gene or transcription unit (SEQ ID NO:49) flanked by nucleic acid molecules comprising lacZ repeats (SEQ ID NO:50) which in turn is flanked on one side by a nucleic acid molecule comprising a nucleotide sequence from the 5′ region of the OCH1 gene (SEQ ID NO:51) and on the other side by a nucleic acid molecule comprising a nucleotide sequence from the 3′ region of the OCH1 gene (SEQ ID NO:52). Plasmid pGLY40 was linearized with SfiI and the linearized plasmid transformed into strain YGLY1-3 to produce a number of strains in which the URA5 gene flanked by the lacZ repeats has been inserted into the OCH1 locus by double-crossover homologous recombination. Strain YGLY2-3 was selected from the strains produced and is prototrophic for URA5. Strain YGLY2-3 was counterselected in the presence of 5-fluoroorotic acid (5-FOA) to produce a number of strains in which the URA5 gene has been lost and only the lacZ repeats remain in the OCH1 locus. This renders the strain auxotrophic for uracil. Strain YGLY4-3 was selected.

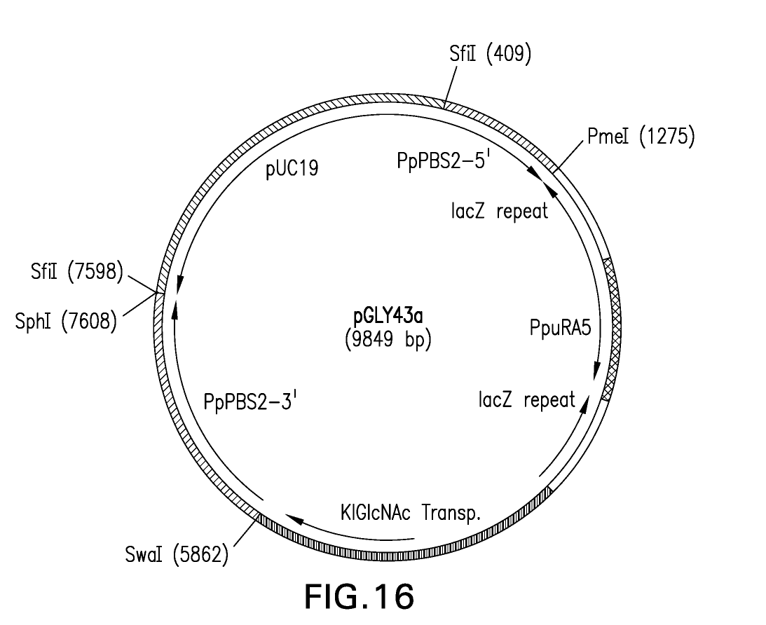

Plasmid pGLY43a (FIG. 16) is an integration vector that targets the BMT2 locus and contains a nucleic acid molecule comprising the K. lactis UDP-N-acetylglucosamine (UDP-GlcNAc) transporter gene or transcription unit (KlMNN2-2, SEQ ID NO:53) adjacent to a nucleic acid molecule comprising the P. pastoris URA5 gene or transcription unit flanked by nucleic acid molecules comprising lacZ repeats. The adjacent genes are flanked on one side by a nucleic acid molecule comprising a nucleotide sequence from the 5′ region of the BMT2 gene (SEQ ID NO: 54) and on the other side by a nucleic acid molecule comprising a nucleotide sequence from the 3′ region of the BMT2 gene (SEQ ID NO:55). Plasmid pGLY43a was linearized with SfiI and the linearized plasmid transformed into strain YGLY4-3 to produce to produce a number of strains in which the KlMNN2-2 gene and URA5 gene flanked by the lacZ repeats has been inserted into the BMT2 locus by double-crossover homologous recombination. The BMT2 gene has been disclosed in Mille et al., J. Biol. Chem. 283: 9724-9736 (2008) and U.S. Pat. No. 7,465,557. Strain YGLY6-3 was selected from the strains produced and is prototrophic for uracil. Strain YGLY6-3 was counterselected in the presence of 5-FOA to produce strains in which the URA5 gene has been lost and only the lacZ repeats remain. This renders the strain auxotrophic for uracil. Strain YGLY8-3 was selected.

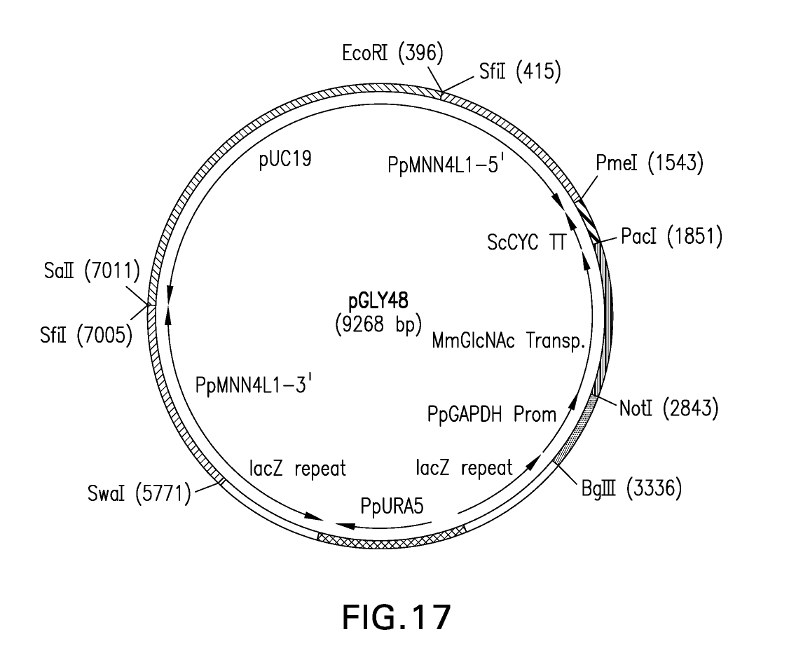

Plasmid pGLY48 (FIG. 17) is an integration vector that targets the MNN4L1 locus and contains an expression cassette comprising a nucleic acid molecule encoding the mouse homologue of the UDP-GlcNAc transporter (SEQ ID NO:56) open reading frame (ORF) operably linked at the 5′ end to a nucleic acid molecule comprising the P. pastoris GAPDH promoter (SEQ ID NO:57) and at the 3′ end to a nucleic acid molecule comprising the S. cerevisiae CYC termination sequences (SEQ ID NO:58) adjacent to a nucleic acid molecule comprising the P. pastoris URA5 gene flanked by lacZ repeats and in which the expression cassettes together are flanked on one side by a nucleic acid molecule comprising a nucleotide sequence from the 5′ region of the P. pastoris MNN4L1 gene (SEQ ID NO:59) and on the other side by a nucleic acid molecule comprising a nucleotide sequence from the 3′ region of the MNN4L1 gene (SEQ ID NO:60). Plasmid pGLY48 was linearized with SfiI and the linearized plasmid transformed into strain YGLY8-3 to produce a number of strains in which the expression cassette encoding the mouse UDP-GlcNAc transporter and the URA5 gene have been inserted into the MNN4L1 locus by double-crossover homologous recombination. The MNN4L1 gene (also referred to as MNN4B) has been disclosed in U.S. Pat. No. 7,259,007. Strain YGLY10-3 was selected from the strains produced and then counterselected in the presence of 5-FOA to produce a number of strains in which the URA5 gene has been lost and only the lacZ repeats remain. Strain YGLY12-3 was selected.

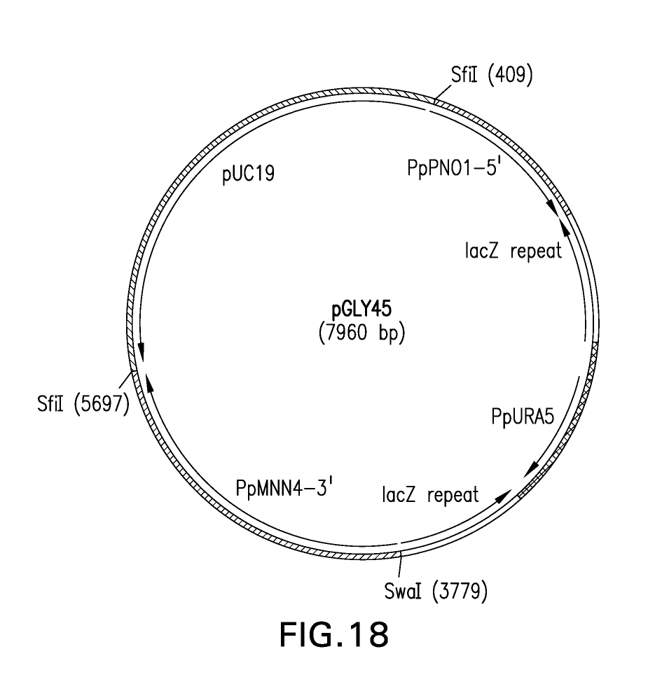

Plasmid pGLY45 (FIG. 18) is an integration vector that targets the PNO1/MNN4 loci and contains a nucleic acid molecule comprising the P. pastoris URA5 gene or transcription unit flanked by nucleic acid molecules comprising lacZ repeats which in turn is flanked on one side by a nucleic acid molecule comprising a nucleotide sequence from the 5′ region of the PNO1 gene (SEQ ID NO:61) and on the other side by a nucleic acid molecule comprising a nucleotide sequence from the 3′ region of the MNN4 gene (SEQ ID NO:62). Plasmid pGLY45 was linearized with SfiI and the linearized plasmid transformed into strain YGLY12-3 to produce a number of strains in which the URA5 gene flanked by the lacZ repeats has been inserted into the PNO1/MNN4 loci by double-crossover homologous recombination. The PNO1 gene has been disclosed in U.S. Pat. No. 7,198,921 and the MNN4 gene (also referred to as MNN4B) has been disclosed in U.S. Pat. No. 7,259,007. Strain YGLY14-3 was selected from the strains produced and then counterselected in the presence of 5-FOA to produce a number of strains in which the URA5 gene has been lost and only the lacZ repeats remain. Strain YGLY16-3 was selected.

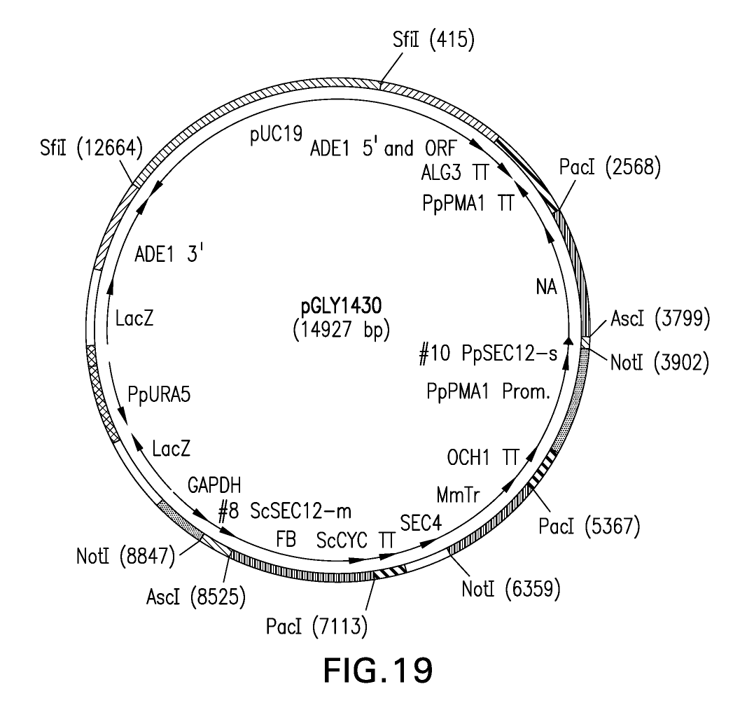

Plasmid pGLY1430 (FIG. 19) is a KINKO integration vector that targets the ADE1 locus without disrupting expression of the locus and contains in tandem four expression cassettes encoding (1) the human GlcNAc transferase I catalytic domain (NA) fused at the N-terminus to P. pastoris SEC12 leader peptide (10) to target the chimeric enzyme to the ER or Golgi, (2) mouse homologue of the UDP-GlcNAc transporter (MmTr), (3) the mouse mannosidase IA catalytic domain (FB) fused at the N-terminus to S. cerevisiae SEC12 leader peptide (8) to target the chimeric enzyme to the ER or Golgi, and (4) the P. pastoris URA5 gene or transcription unit. KINKO (Knock-In with little or No Knock-Out) integration vectors enable insertion of heterologous DNA into a targeted locus without disrupting expression of the gene at the targeted locus and have been described in U.S. Published Application No. 20090124000. The expression cassette encoding the NA10 comprises a nucleic acid molecule encoding the human GlcNAc transferase I catalytic domain codon-optimized for expression in P. pastoris (SEQ ID NO:63) fused at the 5′ end to a nucleic acid molecule encoding the SEC12 leader 10 (SEQ ID NO:64), which is operably linked at the 5′ end to a nucleic acid molecule comprising the P. pastoris PMA1 promoter (SEQ ID NO:65) and at the 3′ end to a nucleic acid molecule comprising the P. pastoris PMA1 transcription termination sequence (SEQ ID NO:66). The expression cassette encoding MmTr comprises a nucleic acid molecule encoding the mouse homologue of the UDP-GlcNAc transporter ORF (SEQ ID NO:56) operably linked at the 5′ end to a nucleic acid molecule comprising the P. pastoris SEC4 promoter (SEQ ID NO:67) and at the 3′ end to a nucleic acid molecule comprising the P. pastoris OCH1 termination sequences (SEQ ID NO:68). The expression cassette encoding the FB8 comprises a nucleic acid molecule encoding the mouse mannosidase IA catalytic domain (SEQ ID NO:69) fused at the 5′ end to a nucleic acid molecule encoding the SEC12-m leader 8 (SEQ ID NO:70), which is operably linked at the 5′ end to a nucleic acid molecule comprising the P. pastoris GADPH promoter and at the 3′ end to a nucleic acid molecule comprising the S. cerevisiae CYC transcription termination sequence. The URA5 expression cassette comprises a nucleic acid molecule comprising the P. pastoris URA5 gene or transcription unit flanked by nucleic acid molecules comprising lacZ repeats. The four tandem cassettes are flanked on one side by a nucleic acid molecule comprising a nucleotide sequence from the 5′ region and complete ORF of the ADE1 gene (SEQ ID NO:71) followed by a P. pastoris ALG3 termination sequence (SEQ ID NO:72) and on the other side by a nucleic acid molecule comprising a nucleotide sequence from the 3′ region of the ADE1 gene (SEQ ID NO:73). Plasmid pGLY1430 was linearized with SfiI and the linearized plasmid transformed into strain YGLY16-3 to produce a number of strains in which the four tandem expression cassette have been inserted into the ADE1 locus immediately following the ADE1 ORF by double-crossover homologous recombination. The strain YGLY2798 was selected from the strains produced and is auxotrophic for arginine and now prototrophic for uridine, histidine, and adenine. The strain was then counterselected in the presence of 5-FOA to produce a number of strains now auxotrophic for uridine. Strain YGLY3794 was selected and is capable of making glycoproteins that have predominantly galactose terminated N-glycans.

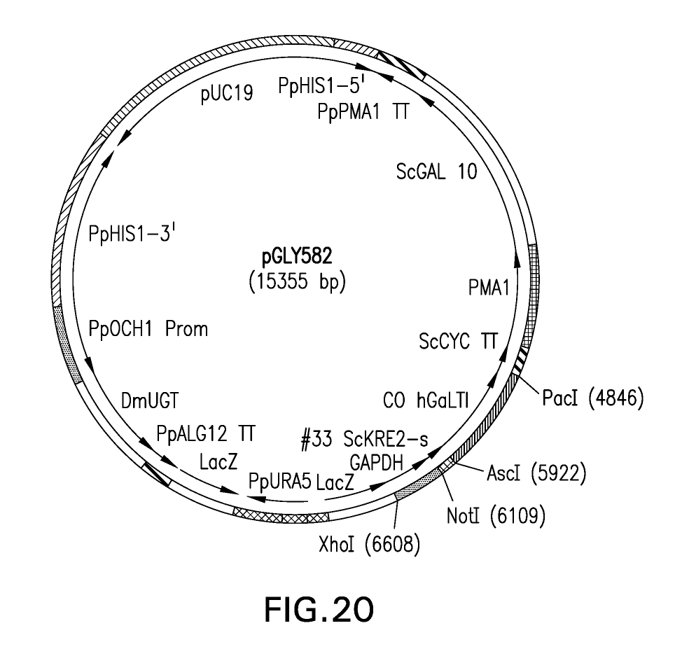

Plasmid pGLY582 (FIG. 20) is an integration vector that targets the HIS1 locus and contains in tandem four expression cassettes encoding (1) the S. cerevisiae UDP-glucose epimerase (ScGAL10), (2) the human galactosyltransferase I (hGalT) catalytic domain fused at the N-terminus to the S. cerevisiae KRE2-s leader peptide (33) to target the chimeric enzyme to the ER or Golgi, (3) the P. pastoris URA5 gene or transcription unit flanked by lacZ repeats, and (4) the D. melanogaster UDP-galactose transporter (DmUGT). The expression cassette encoding the ScGAL10 comprises a nucleic acid molecule encoding the ScGAL10 ORF (SEQ ID NO:74) operably linked at the 5′ end to a nucleic acid molecule comprising the P. pastoris PMA1 promoter (SEQ ID NO:65) and operably linked at the 3′ end to a nucleic acid molecule comprising the P. pastoris PMA1 transcription termination sequence (SEQ ID NO:66). The expression cassette encoding the chimeric galactosyltransferase I comprises a nucleic acid molecule encoding the hGalT catalytic domain codon optimized for expression in P. pastoris (SEQ ID NO:75) fused at the 5′ end to a nucleic acid molecule encoding the KRE2-s leader 33 (SEQ ID NO:76), which is operably linked at the 5′ end to a nucleic acid molecule comprising the P. pastoris GAPDH promoter and at the 3′ end to a nucleic acid molecule comprising the S. cerevisiae CYC transcription termination sequence. The URA5 expression cassette comprises a nucleic acid molecule comprising the P. pastoris URA5 gene or transcription unit flanked by nucleic acid molecules comprising lacZ repeats. The expression cassette encoding the DmUGT comprises a nucleic acid molecule encoding the DmUGT ORF (SEQ ID NO:77) operably linked at the 5′ end to a nucleic acid molecule comprising the P. pastoris OCH1 promoter (SEQ ID NO:78) and operably linked at the 3′ end to a nucleic acid molecule comprising the P. pastoris ALG12 transcription termination sequence (SEQ ID NO:79). The four tandem cassettes are flanked on one side by a nucleic acid molecule comprising a nucleotide sequence from the 5′ region of the HIS1 gene (SEQ ID NO:80) and on the other side by a nucleic acid molecule comprising a nucleotide sequence from the 3′ region of the HIS1 gene (SEQ ID NO:81). Plasmid pGLY582 was linearized and the linearized plasmid transformed into strain YGLY3794 to produce a number of strains in which the four tandem expression cassette have been inserted into the HIS1 locus by homologous recombination. Strain YGLY3853 was selected and is auxotrophic for histidine and prototrophic for uridine.

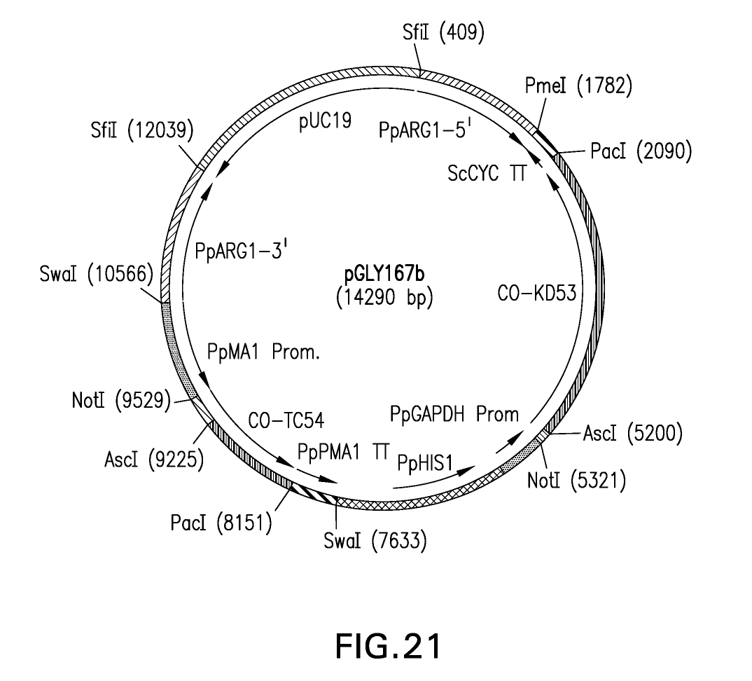

Plasmid pGLY167b (FIG. 21) is an integration vector that targets the ARG1 locus and contains in tandem three expression cassettes encoding (1) the D. melanogaster mannosidase II catalytic domain (KD) fused at the N-terminus to S. cerevisiae MNN2 leader peptide (53) to target the chimeric enzyme to the ER or Golgi, (2) the P. pastoris HIS1 gene or transcription unit, and (3) the rat N-acetylglucosamine (GlcNAc) transferase II catalytic domain (TC) fused at the N-terminus to S. cerevisiae MNN2 leader peptide (54) to target the chimeric enzyme to the ER or Golgi. The expression cassette encoding the KD53 comprises a nucleic acid molecule encoding the D. melanogaster mannosidase II catalytic domain codon-optimized for expression in P. pastoris (SEQ ID NO:82) fused at the 5′ end to a nucleic acid molecule encoding the MNN2 leader 53 (SEQ ID NO:83), which is operably linked at the 5′ end to a nucleic acid molecule comprising the P. pastoris GAPDH promoter and at the 3′ end to a nucleic acid molecule comprising the S. cerevisiae CYC transcription termination sequence. The HIS1 expression cassette comprises a nucleic acid molecule comprising the P. pastoris HIS1 gene or transcription unit (SEQ ID NO:84). The expression cassette encoding the TC54 comprises a nucleic acid molecule encoding the rat GlcNAc transferase II catalytic domain codon-optimized for expression in P. pastoris (SEQ ID NO:85) fused at the 5′ end to a nucleic acid molecule encoding the MNN2 leader 54 (SEQ ID NO:86), which is operably linked at the 5′ end to a nucleic acid molecule comprising the P. pastoris PMA1 promoter and at the 3′ end to a nucleic acid molecule comprising the P. pastoris PMA1 transcription termination sequence. The three tandem cassettes are flanked on one side by a nucleic acid molecule comprising a nucleotide sequence from the 5′ region of the ARG1 gene (SEQ ID NO:87) and on the other side by a nucleic acid molecule comprising a nucleotide sequence from the 3′ region of the ARG1 gene (SEQ ID NO:88). Plasmid pGLY167b was linearized with SfiI and the linearized plasmid transformed into strain YGLY3853 to produce a number of strains (in which the three tandem expression cassette have been inserted into the ARG1 locus by double-crossover homologous recombination. The strain YGLY4754 was selected from the strains produced and is auxotrophic for arginine and prototrophic for uridine and histidine. The strain was then counterselected in the presence of 5-FOA to produce a number of strains now auxotrophic for uridine. Strain YGLY4799 was selected.

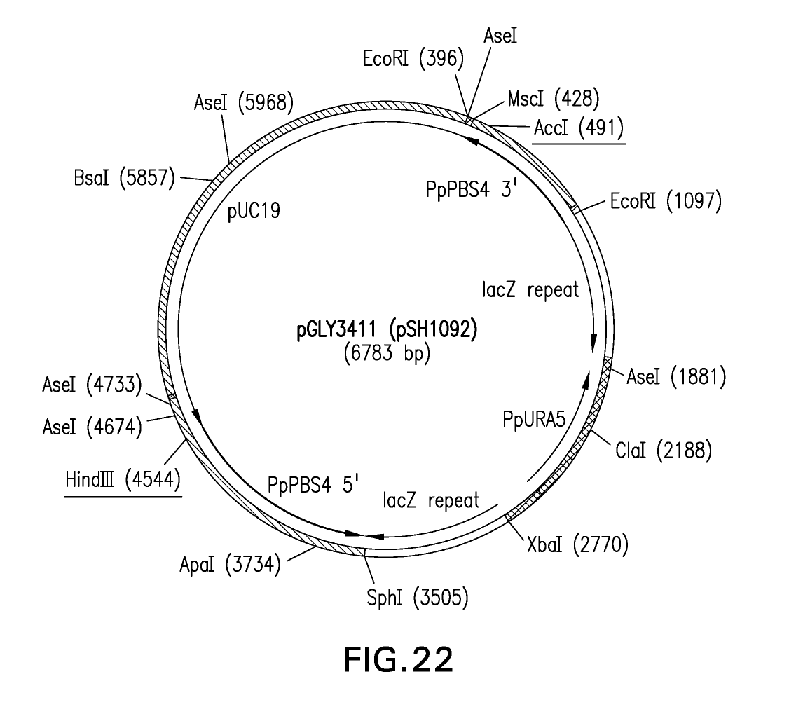

Plasmid pGLY3411 (FIG. 22) is an integration vector that contains the expression cassette comprising the P. pastoris URA5 gene flanked by lacZ repeats flanked on one side with the 5′ nucleotide sequence of the P. pastoris BMT4 gene (SEQ ID NO:89) and on the other side with the 3′ nucleotide sequence of the P. pastoris BMT4 gene (SEQ ID NO:90). Plasmid pGLY3411 was linearized and the linearized plasmid transformed into YGLY4799 to produce a number of strains in which the URA5 expression cassette has been inserted into the BMT4 locus by double-crossover homologous recombination. Strain YGLY6903 was selected from the strains produced and is prototrophic for uracil, adenine, histidine, proline, arginine, and tryptophan. The strain was then counterselected in the presence of 5-FOA to produce a number of strains now auxotrophic for uridine. Strains YGLY7432 and YGLY7433 were selected.

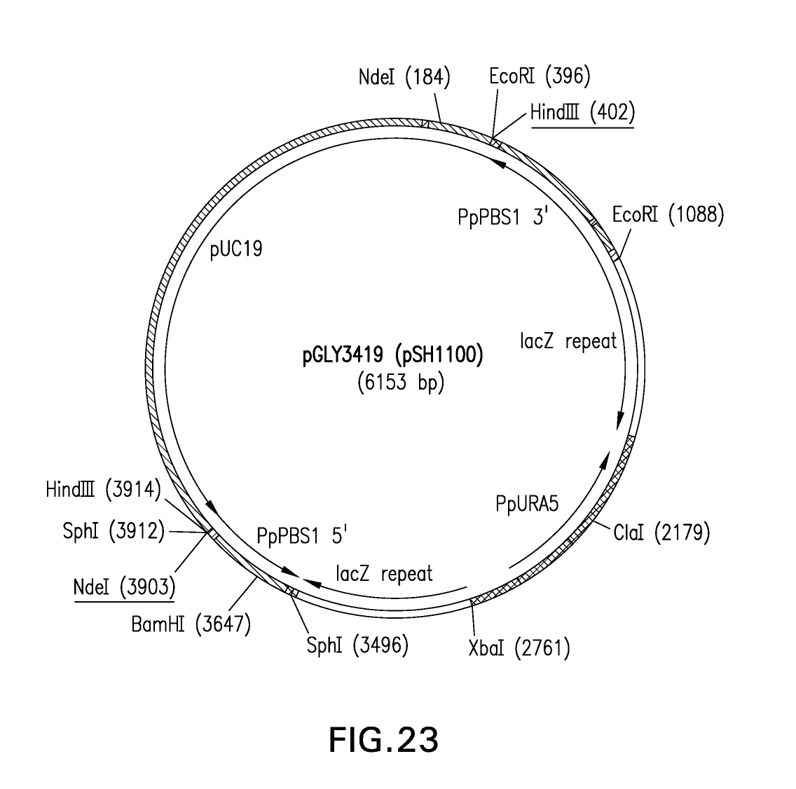

Plasmid pGLY3419 (FIG. 23) is an integration vector that contains an expression cassette comprising the P. pastoris URA5 gene flanked by lacZ repeats flanked on one side with the 5′ nucleotide sequence of the P. pastoris BMT1 gene (SEQ ID NO:91) and on the other side with the 3′ nucleotide sequence of the P. pastoris BMT1 gene (SEQ ID NO:92). Plasmid pGLY3419 was linearized and the linearized plasmid transformed into strain YGLY7432 and YGLY7433 to produce a number of strains in which the URA5 expression cassette has been inserted into the BMT1 locus by double-crossover homologous recombination. The strains YGLY7651 and YGLY7656 were selected from the strains produced and are prototrophic for uracil, adenine, histidine, proline, arginine, and tryptophan. The strains were then counterselected in the presence of 5-FOA to produce a number of strains now auxotrophic for uridine. Strains YGLY7930 and YGLY7940 were selected.

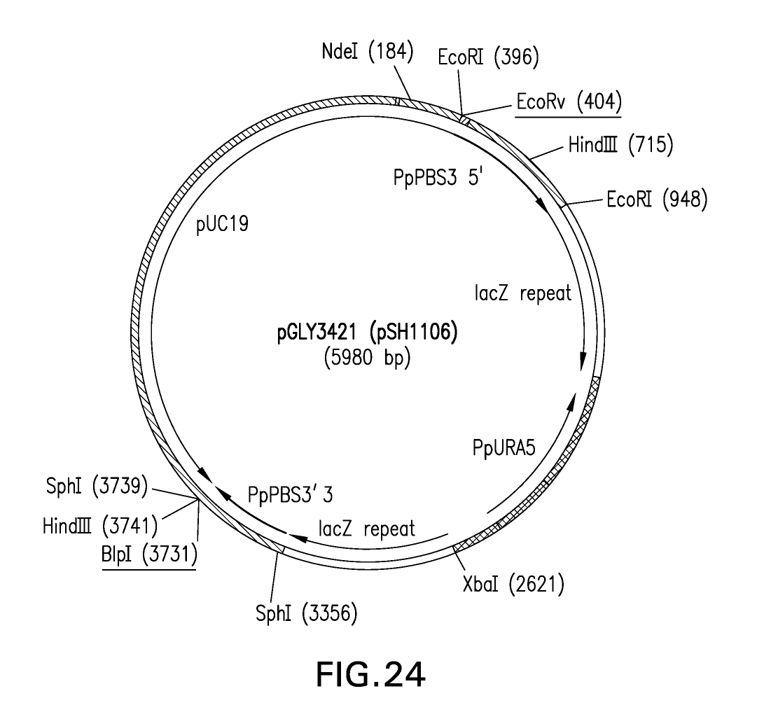

Plasmid pGLY3421 (FIG. 24) is an integration vector that contains an expression cassette comprising the P. pastoris URA5 gene flanked by lacZ repeats flanked on one side with the 5′ nucleotide sequence of the P. pastoris BMT3 gene (SEQ ID NO:93) and on the other side with the 3′ nucleotide sequence of the P. pastoris BMT3 gene (SEQ ID NO:94). Plasmid pGLY3419 was linearized and the linearized plasmid transformed into strain YGLY7930 and YGLY7940 to produce a number of strains in which the URA5 expression cassette has been inserted into the BMT1 locus by double-crossover homologous recombination. Strains YGLY7961 and YGLY7965 were selected from the strains produced and are prototrophic for uracil, adenine, histidine, proline, arginine, and tryptophan.

Plasmid pGLY2456 (FIG. 25) is a KINKO integration vector that targets the TRP2 locus without disrupting expression of the locus and contains six expression cassettes encoding (1) the mouse CMP-sialic acid transporter (mCMP-Sia Transp), (2) the human UDP-GlcNAc 2-epimerase/N-acetylmannosamine kinase (hGNE), (3) the Pichia pastoris ARG1 gene or transcription unit, (4) the human CMP-sialic acid synthase (hCSS), (5) the human N-acetylneuraminate-9-phosphate synthase (hSPS), (6) the mouse α-2,6-sialyltransferase catalytic domain (mST6) fused at the N-terminus to S. cerevisiae KRE2 leader peptide (33) to target the chimeric enzyme to the ER or Golgi, and the P. pastoris ARG1 gene or transcription unit. The expression cassette encoding the mouse CMP-sialic acid transporter comprises a nucleic acid molecule encoding the mCMP Sia Transp ORF codon optimized for expression in P. pastoris (SEQ ID NO:95), which is operably linked at the 5′ end to a nucleic acid molecule comprising the P. pastoris PMA-1 promoter and at the 3′ end to a nucleic acid molecule comprising the P. pastoris PMA1 transcription termination sequence. The expression cassette encoding the human UDP-GlcNAc 2-epimerase/N-acetylmannosamine kinase comprises a nucleic acid molecule encoding the hGNE ORF codon optimized for expression in P. pastoris (SEQ ID NO:96), which is operably linked at the 5′ end to a nucleic acid molecule comprising the P. pastoris GAPDH promoter and at the 3′ end to a nucleic acid molecule comprising the S. cerevisiae CYC transcription termination sequence. The expression cassette encoding the P. pastoris ARG1 gene comprises (SEQ ID NO:97). The expression cassette encoding the human CMP-sialic acid synthase comprises a nucleic acid molecule encoding the hCSS ORF codon optimized for expression in P. pastoris (SEQ ID NO:98), which is operably linked at the 5′ end to a nucleic acid molecule comprising the P. pastoris GAPDH promoter and at the 3′ end to a nucleic acid molecule comprising the S. cerevisiae CYC transcription termination sequence. The expression cassette encoding the human N-acetylneuraminate-9-phosphate synthase comprises a nucleic acid molecule encoding the hSIAP S ORF codon optimized for expression in P. pastoris (SEQ ID NO:99), which is operably linked at the 5′ end to a nucleic acid molecule comprising the P. pastoris PMA-1 promoter and at the 3′ end to a nucleic acid molecule comprising the P. pastoris PMA1 transcription termination sequence. The expression cassette encoding the chimeric mouse α-2,6-sialyltransferase comprises a nucleic acid molecule encoding the mST6 catalytic domain codon optimized for expression in P. pastoris (SEQ ID NO:100) fused at the 5′ end to a nucleic acid molecule encoding the S. cerevisiae KRE2 signal peptide, which is operably linked at the 5′ end to a nucleic acid molecule comprising the P. pastoris TEF promoter and at the 3′ end to a nucleic acid molecule comprising the P. pastoris TEF transcription termination sequence. The six tandem cassettes are flanked on one side by a nucleic acid molecule comprising a nucleotide sequence from the 5′ region and ORF of the TRP2 gene ending at the stop codon (SEQ ID NO:101) followed by a P. pastoris ALG3 termination sequence and on the other side by a nucleic acid molecule comprising a nucleotide sequence from the 3′ region of the TRP2 gene (SEQ ID NO:102). Plasmid pGLY2456 was linearized with SfiI and the linearized plasmid transformed into strain YGLY7961 to produce a number of strains in which the six expression cassette have been inserted into the TRP2 locus immediately following the TRP2 ORF by double-crossover homologous recombination. The strain YGLY8146 was selected from the strains produced. The strain was then counterselected in the presence of 5-FOA to produce a number of strains now auxotrophic for uridine. Strain YGLY9296 was selected.

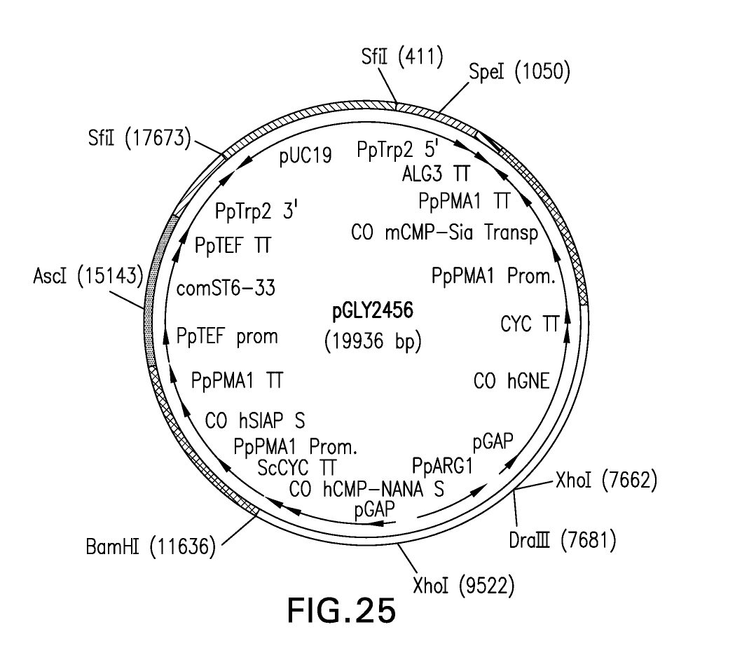

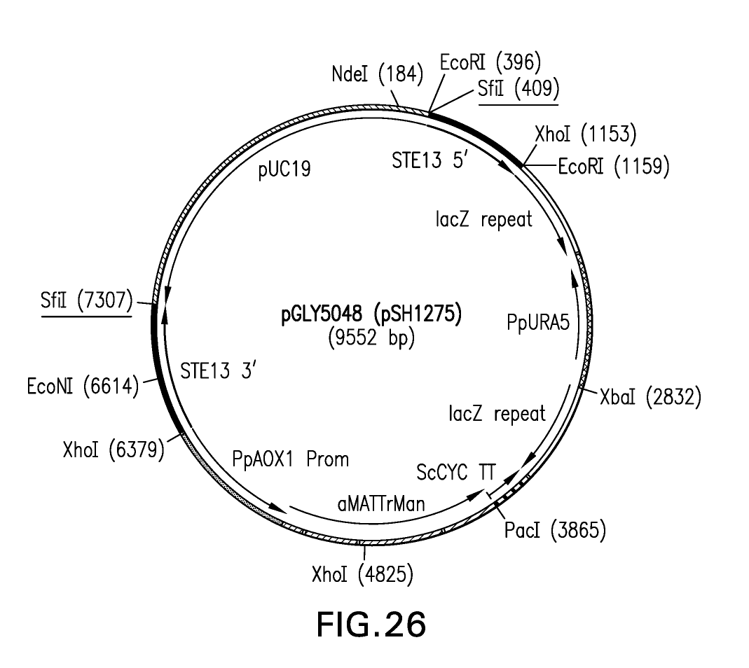

Plasmid pGLY5048 (FIG. 26) is an integration vector that targets the STE13 locus and contains expression cassettes encoding (1) the T. reesei α-1,2-mannosidase catalytic domain fused at the N-terminus to S. cerevisiae αMATpre signal peptide (aMATTrMan) to target the chimeric protein to the secretory pathway and secretion from the cell and (2) the P. pastoris URA5 gene or transcription unit. The expression cassette encoding the aMATTrMan comprises a nucleic acid molecule encoding the T. reesei catalytic domain (SEQ ID NO:103) fused at the 5′ end to a nucleic acid molecule encoding the S. cerevisiae αMATpre signal peptide (SEQ ID NO:104 encoding amino acid sequence SEQ ID NO:105), which is operably linked at the 5′ end to a nucleic acid molecule comprising the P. pastoris AOX1 promoter and at the 3′ end to a nucleic acid molecule comprising the S. cerevisiae CYC transcription termination sequence. The URA5 expression cassette comprises a nucleic acid molecule comprising the P. pastoris URA5 gene or transcription unit flanked by nucleic acid molecules comprising lacZ repeats. The two tandem cassettes are flanked on one side by a nucleic acid molecule comprising a nucleotide sequence from the 5′ region of the STE13 gene (SEQ ID NO:106) and on the other side by a nucleic acid molecule comprising a nucleotide sequence from the 3′ region of the STE13 gene (SEQ ID NO:107). Plasmid pGLY5048 was linearized with SfiI and the linearized plasmid transformed into strain YGLY9296 to produce a number of strains. The strains YGLY9469 and YGLY9465 were selected from the strains produced. The strains are capable of producing glycoproteins that have single-mannose O-glycosylation (See Published U.S. Application No. 20090170159).

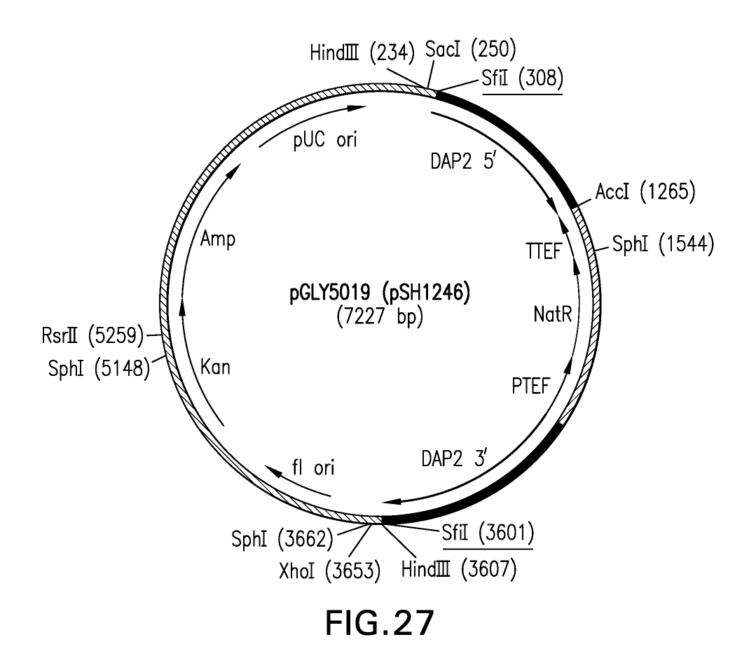

Plasmid pGLY5019 (FIG. 27) is an integration vector that targets the DAP2 locus and contains an expression cassette comprising a nucleic acid molecule encoding the Nourseothricin resistance (NATR) expression cassette (originally from pAG25 from EROSCARF, Scientific Research and Development GmbH, Daimlerstrasse 13a, D-61352 Bad Homburg, Germany, See Goldstein et al., Yeast 15: 1541 (1999); GenBank Accession Nos. CAR31387.1 and CAR31383.1). The NAT^{R} expression cassette (SEQ ID NO:108) is operably regulated to the Ashbya gossypii TEF1 promoter (SEQ ID NO:109) and A. gossypii TEF1 termination sequence (SEQ ID NO:110) flanked one side with the 5′ nucleotide sequence of the P. pastoris DAP2 gene (SEQ ID NO:111) and on the other side with the 3′ nucleotide sequence of the P. pastoris DAP2 gene (SEQ ID NO:112). Plasmid pGLY5019 was linearized and the linearized plasmid transformed into strain YGLY9469 to produce a number of strains in which the NATR expression cassette has been inserted into the DAP2 locus by double-crossover homologous recombination. The strain YGLY9797 was selected from the strains produced.

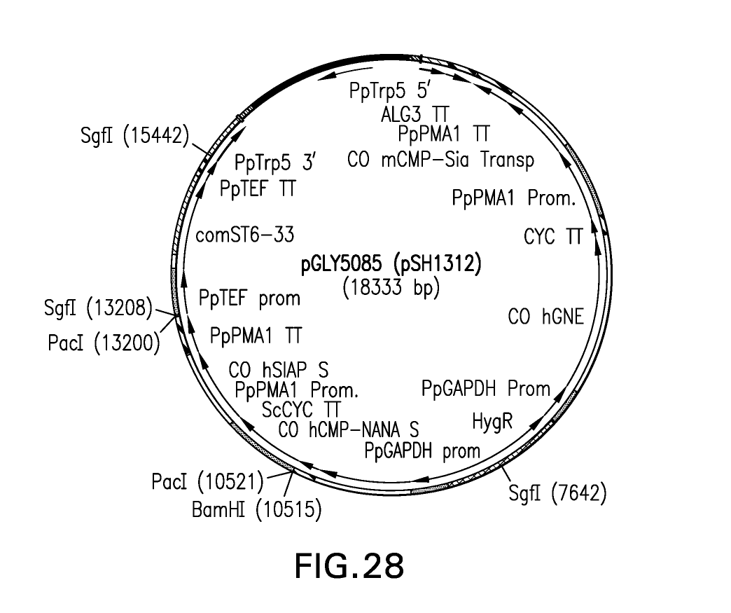

Plasmid pGLY5085 (FIG. 28) is a KINKO plasmid for introducing a second set of the genes involved in producing sialylated N-glycans into P. pastoris. The plasmid is similar to plasmid YGLY2456 except that the P. pastoris ARG1 gene has been replaced with an expression cassette encoding hygromycin resistance (HygR) and the plasmid targets the P. pastoris TRP5 locus. The HYG^{R} resistance cassette is SEQ ID NO:113. The HYG^{R} expression cassette (SEQ ID NO:113) is operably regulated to the Ashbya gossypii TEF1 promoter and A. gossypii TEF1 termination sequences (See Goldstein et al., Yeast 15: 1541 (1999)). The six tandem cassettes are flanked on one side by a nucleic acid molecule comprising a nucleotide sequence from the 5′ region and ORF of the TRP5 gene ending at the stop codon (SEQ ID NO:114) followed by a P. pastoris ALG3 termination sequence and on the other side by a nucleic acid molecule comprising a nucleotide sequence from the 3′ region of the TRP5 gene (SEQ ID NO:115). Plasmid pGLY5085 was transformed into strain YGLY9797 to produce a number of strains of which strain YGLY12900 and YGL12897 were selected.

### Example 3

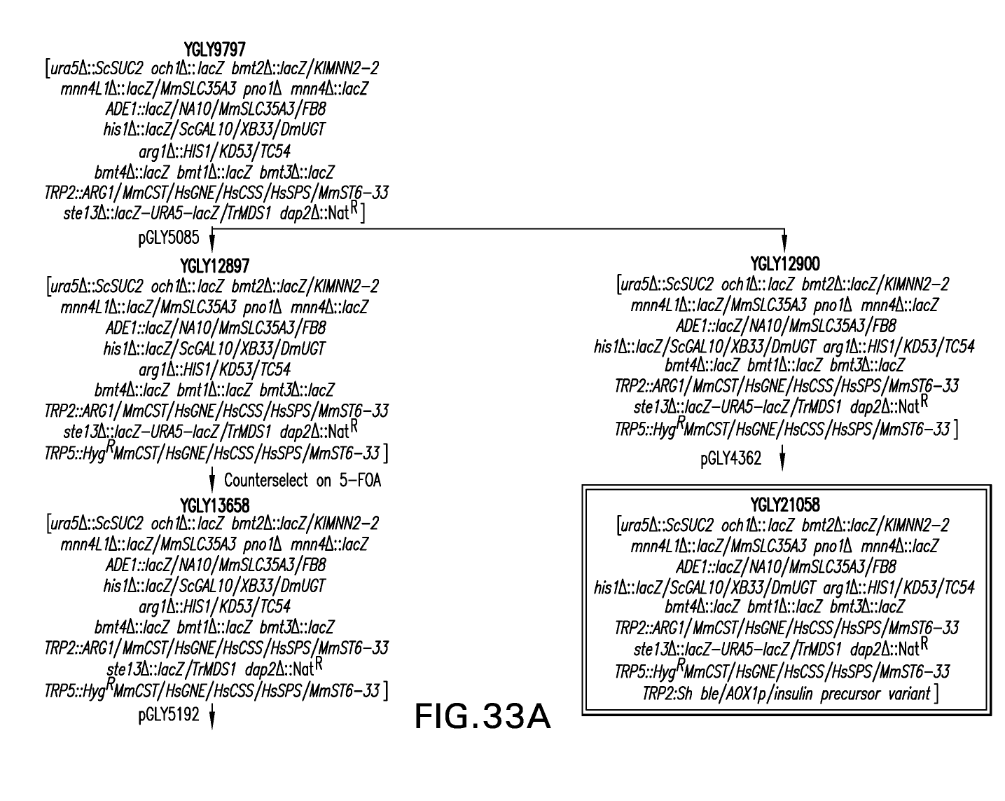

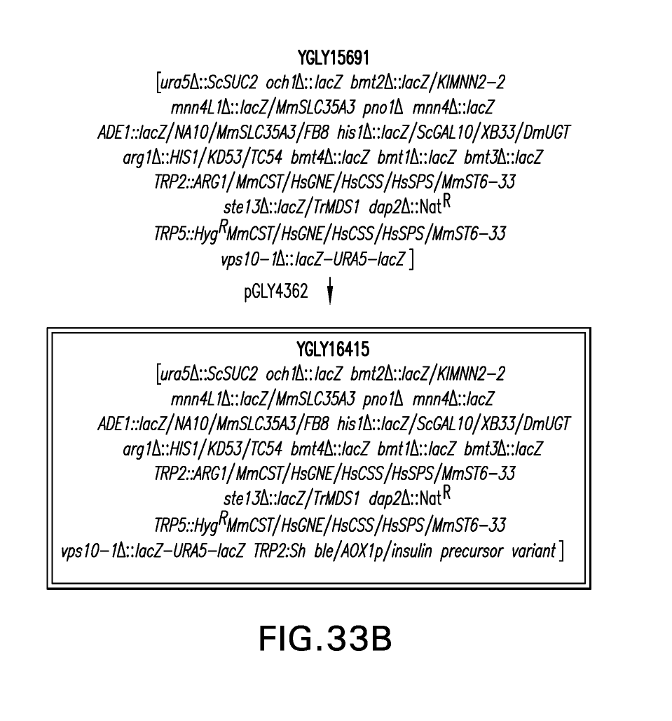

This example describes construction of strains YGLY21058 and YGLY16415. Both strains are capable of producing glycoproteins having sialylated N-glycans and expressing the insulin analogue comprising an N-glycosylation site on the B-chain at position 28 encoded by the expression cassette in plasmid pGLY4362. Construction of the strains from YGLY9797 is shown in FIG. 33A-33B.

Strain YGLY12900 from Example 2 was transformed with plasmid pGLY4362, which is an expression plasmid that in Pichia pastoris enables expression of a glycosylated insulin analogue precursor molecule comprising the Yps1ss domain fused to the TA57 propeptide domain fused to an N-terminal spacer fused to the human insulin B-chain having a P28N substitution fused to a C-peptide having the amino acid sequence AAK fused to the human insulin A-chain, to produce a number of strains of which strain YGLY21058 was selected. The strain is capable of producing an N-glycosylated insulin analogue precursor comprising an N-terminal spacer fused to the human insulin B-chain having a P28N substitution fused to a C-peptide having the amino acid sequence AAK fused to the human insulin A-chain.

Strain YGLY12897 from Example 2 was counterselected in the presence of 5-FOA to produce a number of strains now auxotrophic for uridine of which strain YGLY13658 was selected.

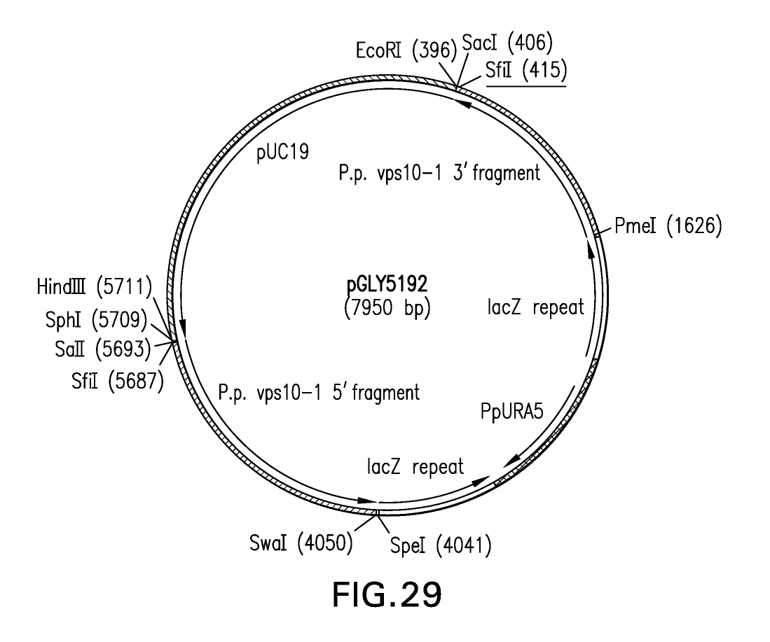

Plasmid pYGLY5192 (FIG. 29) is an integration vector constructed to delete the ORF of the VPS10-1 gene to render the strain deficient in vacuolar sorting receptor (Vps10-1p) activity. The plasmid contains a nucleic acid molecule comprising the P. pastoris URA5 gene or transcription unit (SEQ ID NO:49) flanked by nucleic acid molecules comprising lacZ repeats (SEQ ID NO:50) which in turn is flanked on one side by a nucleic acid molecule comprising a nucleotide sequence from the 5′ region of the VPS10-1 gene (SEQ ID NO:117) and on the other side by a nucleic acid molecule comprising a nucleotide sequence from the 3′ region of the VPS10-1 gene (SEQ ID NO:116). Plasmid was linearized with SfiI and the linearized plasmid transformed into strain YGLY13658 to produce a number of strains of which strain YGLY15691 was selected. Strain YGLY15691 was transformed with plasmid pGLY4362 to produce a number of strains of which strain YGLY16415 was selected. The strain is capable of producing an N-glycosylated insulin analogue precursor comprising an N-terminal spacer fused to the human insulin B-chain having a P28N substitution fused to a C-peptide having the amino acid sequence AAK fused to the human insulin A-chain.

### Example 4

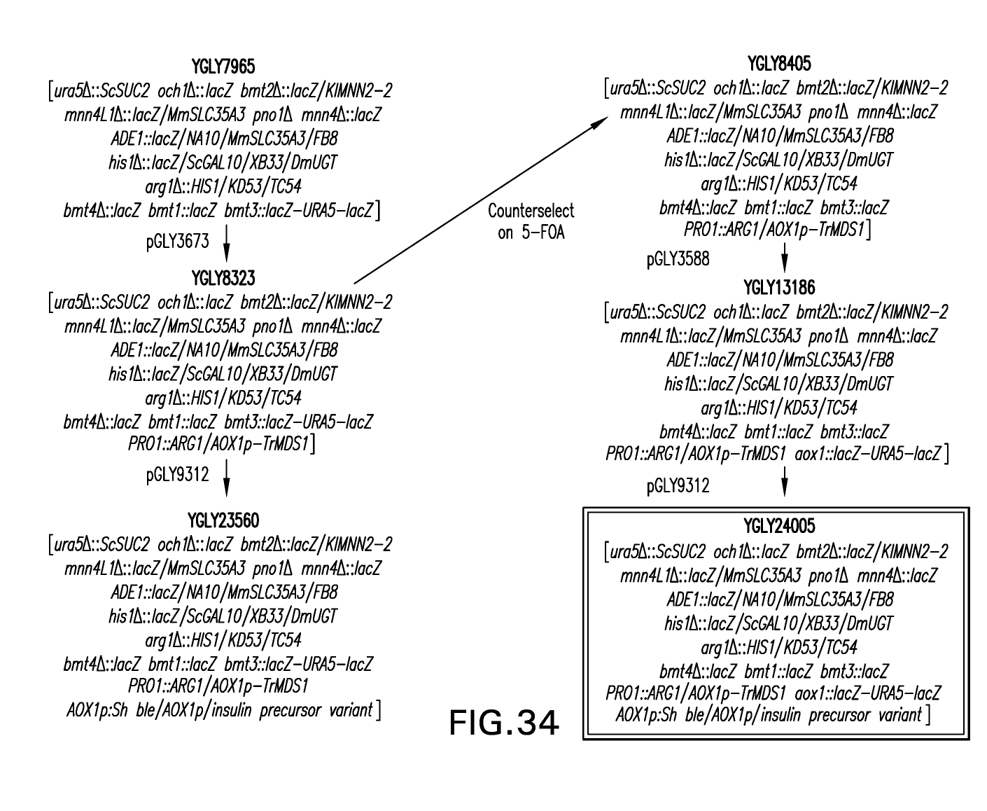

This example describes construction of strains YGLY23560 and YGLY24005. Both strains are capable of producing glycoproteins having galactose-terminated N-glycans and expressing an insulin analogue comprising an N-glycosylation site on the B-chain at position 28 encoded by the expression cassette in plasmid pGLY9312. Construction of the strains from strain YGLY7965 is shown in FIG. 34.

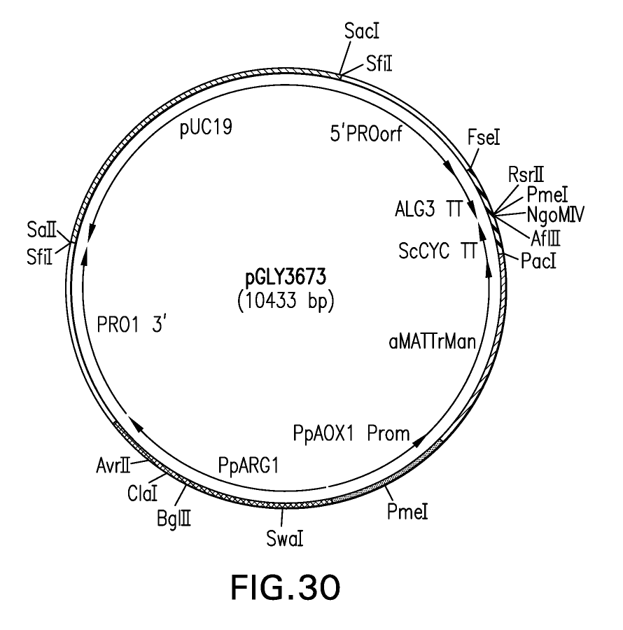

Plasmid pGLY3673 (FIG. 30) is a KINKO integration vector that targets the PRO1 locus without disrupting expression of the locus and contains expression cassettes encoding the T. reesei α-1,2-mannosidase catalytic domain fused at the N-terminus to S. cerevisiae αMATpre signal peptide (aMATTrMan) to target the chimeric protein to the secretory pathway and secretion from the cell. The expression cassette encoding the aMATTrMan comprises a nucleic acid molecule encoding the T. reesei catalytic domain (SEQ ID NO:103) fused at the 5′ end to a nucleic acid molecule encoding the S. cerevisiae αMATpre signal peptide (SEQ ID NO:104), which is operably linked at the 5′ end to a nucleic acid molecule comprising the P. pastoris AOX1 promoter (SEQ ID NO:118) and at the 3′ end to a nucleic acid molecule comprising the S. cerevisiae CYC transcription termination sequence (SEQ ID NO:58). The cassette is flanked on one side by a nucleic acid molecule comprising a nucleotide sequence from the 5′ region and complete ORF of the PRO1 gene (SEQ ID NO:119) followed by a P. pastoris ALG3 termination sequence and on the other side by a nucleic acid molecule comprising a nucleotide sequence from the 3′ region of the PRO1 gene (SEQ ID NO:120). The plasmid contains the PpARG1 gene. Plasmid pGLY3673 was transformed into strain YGLY7965 from Example 2 to produce a number strains of which strain YGLY8323 was selected.

To make strain YGLY23560, strain YGLY8323 was transformed with plasmid pGLY9312, which is an expression plasmid that in Pichia pastoris enables expression of a glycosylated insulin analogue precursor molecule comprising the S. cerevisiae alpha mating factor signal sequence and pro-peptide fused to an N-terminal MYC spacer peptide fused to a human insulin B-chain having a P28N substitution fused to a C-peptide “TA(10×HIS)AK” fused to a human insulin A-chain, to produce a number of strains of which strain YGLY23560 was selected. The strain is capable of producing an N-glycosylated insulin analogue precursor comprising an N-terminal MYC spacer peptide fused to a human insulin B-chain having a P28N substitution fused to a C-peptide “TA(10×HIS)AK” fused to a human insulin A-chain.

To make strain YGLY24005, strain YGLY8323 was counterselected in the presence of 5-FOA to produce a number of strains now auxotrophic for uridine of which strain YGLY8405 was selected.

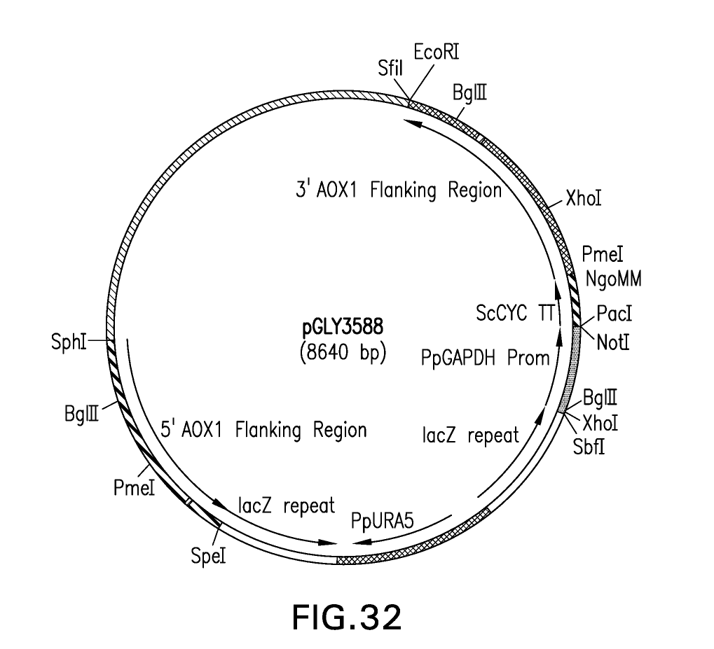

Plasmid pYGLY3588 (FIG. 32) is an integration vector that targets the AOX1 locus and carries the Pichia pastoris URA5 gene or transcription unit (PpURA5) flanked by nucleic acid molecules comprising lacZ repeats (lacZ repeat) (See plasmid pYGLY6) which in turn is flanked on one side by a nucleic acid molecule comprising a nucleotide sequence from the 5′ region of the AOX1 gene (SEQ ID NO:124) and on the other side by a nucleic acid molecule comprising a nucleotide sequence from the 3′ region of the AOX1 gene (SEQ ID NO:125).

Plasmid pGLY3588 was transformed into strain YGLY8405 to produce a number of strains that were prototrophic for uridine of which strain YGLY13186 was selected. Strain

YGLY13186 was transformed with plasmid pGLY9312 to produce a number of strains of which strain YGLY24005 was selected. The strain is capable of producing an N-glycosylated insulin analogue precursor comprising the an N-terminal MYC spacer peptide fused to a human insulin B-chain having a P28N substitution fused to a C-peptide “TA(10×HIS)AK” fused to a human insulin A-chain.

### Example 5

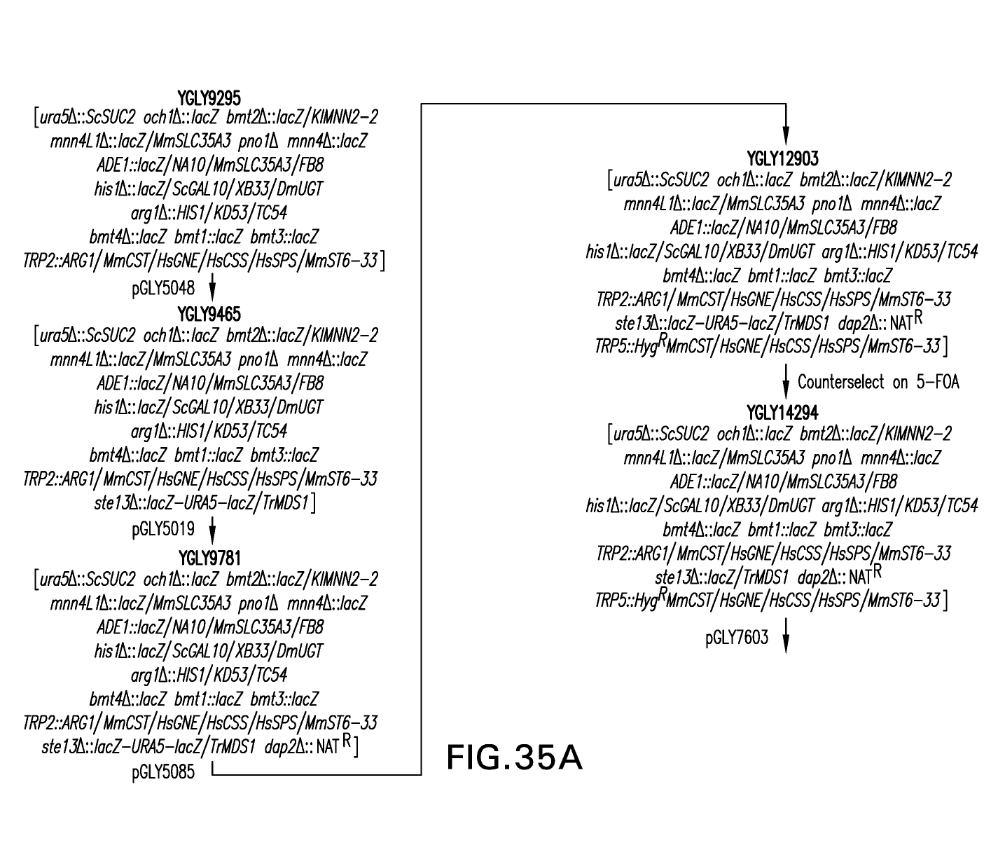

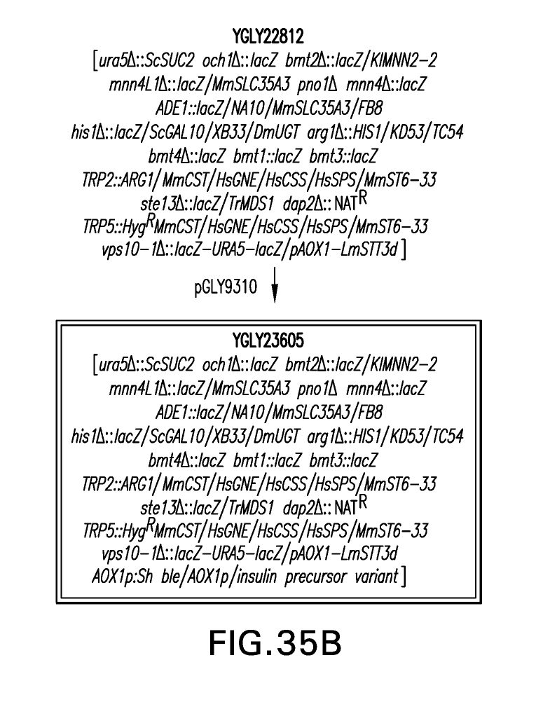

This example describes construction of strain YGLY23605 from strain YGLY9465 of Example 2. The strain is capable of producing glycoproteins having sialylated N-glycans and expressing an insulin analogue comprising an N-glycosylation site on the B-chain at position 28 encoded by the expression cassette in plasmid pGLY9312. The strain further includes the Leishmania major STT3D (LmSTT3D) open reading frame (ORF) operably linked to an inducible promoter. Inclusion of the LmSTT3D gene has been shown to increase the N-glycosylation site occupancy (See International Application No. PCT/US2011/025878). Construction of the strain from YGLY9465 is shown in FIG. 35A-B.

Plasmid pGLY5019 as described in Example 2 is an integration vector that targets the DAP2 locus and contains an expression cassette comprising a nucleic acid molecule encoding the Nourseothricin resistance (NATR) expression cassette (originally from pAG25 from EROSCARF, Scientific Research and Development GmbH, Daimlerstrasse 13a, D-61352 Bad Homburg, Germany, See Goldstein et al., Yeast 15: 1541 (1999)). Plasmid pGLY5019 was linearized and the linearized plasmid transformed into strain YGLY9465 to produce a number of strains in which the NATR expression cassette has been inserted into the DAP2 locus by double-crossover homologous recombination. The strain YGLY9781 was selected from the strains produced.

Strain YGLY9781 was transformed with plasmid pGLY5085 (Example 2) to produce number of strains of which strains YGLY12903 and YGLY12905 were selected. Strain YGLY12903 was then counterselected in the presence of 5-FOA to produce a number of strains of which strain YGLY14294 was selected.

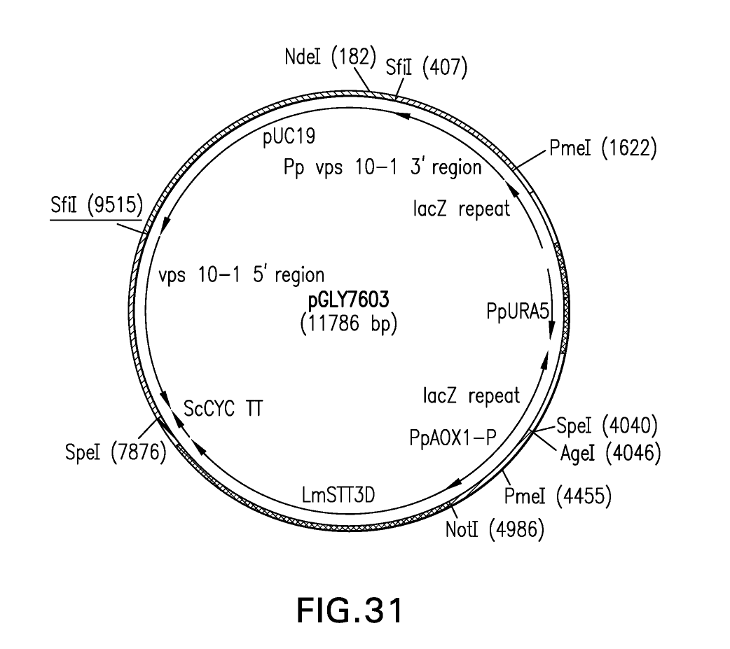

Plasmid pGLY7603 (FIG. 31) is an integration plasmid that targets the VPS10-1 locus in P. pastoris. The expression cassette encoding the LmSTT3D comprises a nucleic acid molecule encoding the LmSTT3D ORF codon-optimized for optimal expression in P. pastoris (SEQ ID NO:121) operably linked at the 5′ end to a nucleic acid molecule that has the inducible P. pastoris AOX1 promoter sequence (SEQ ID NO:118) and at the 3′ end to a nucleic acid molecule that has the S. cerevisiae CYC transcription termination sequence (SEQ ID NO:58) and for selection, the plasmid contains a nucleic acid molecule comprising the P. pastoris URA5 gene or transcription unit (SEQ ID NO:49) flanked by nucleic acid molecules comprising lacZ repeats (SEQ ID NO:50). Both cassettes are flanked on one side by a nucleic acid molecule comprising a nucleotide sequence from the 5′ region of the VPS10-1 gene (SEQ ID NO:117) and on the other side by a nucleic acid molecule comprising a nucleotide sequence from the 3′ region of the VPS10-1 gene (SEQ ID NO:116).

Plasmid pGLY7603 was transformed into strain YGLY14294 to produce number of strains of which strain YGLY22812 was selected.

Strain YGLY22812 was transformed with plasmid pGLY9310 to produce a number of strains of which strain YGLY23605 was selected. The strain is capable of producing an N-glycosylated insulin analogue precursor comprising the human insulin B-chain containing the substitution P28N fused to a C-peptide RR fused to the human insulin A-chain containing an N21G substitution.

### Example 6

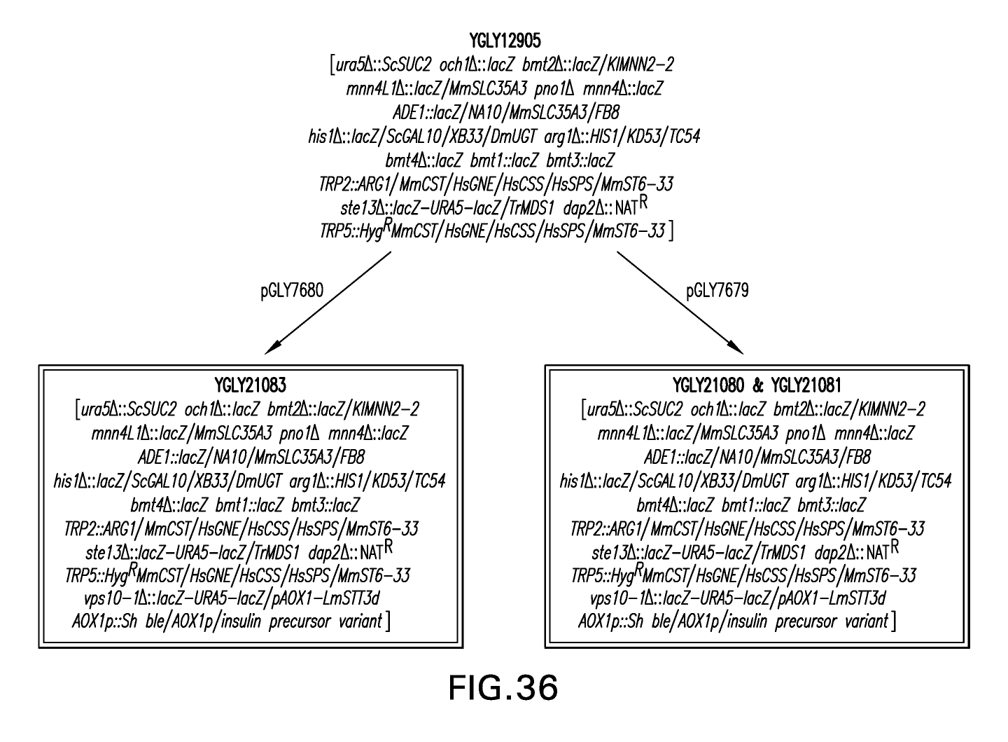

This example describes construction of strains YGLY21083 and YGLY21080 from strain YGLY12905 of Example 5. The strains are capable of producing glycoproteins having sialylated N-glycans and expressing an insulin analogue comprising an N-glycosylation site on the B-chain at position 28 encoded by the expression cassette in plasmid pGLY9312. Construction of the strain from YGLY12905 is shown in FIG. 36.

Strain YGLY12905 was transformed with plasmid pGLY7680 to produce a number of strains of which strain YGLY21083 was selected. The strain is capable of producing a glycosylated proinsulin analogue comprising the human insulin B-chain containing the substitution P28N fused to a C-peptide RR fused to the human insulin A-chain.

Strain YGLY12905 was also transformed with plasmid pGLY7679 to produce a number of strains of which strain YGLY21080 and YGLY21081 were selected. The strain is capable of producing an N-glycosylated insulin analogue precursor comprising an N-terminal spacer peptide fused to the human insulin B-chain containing the substitution P28N fused C-peptide A(10×HIS)AK fused to the human insulin A-chain.

### Example 7

The strains capable of producing the various N-glycosylated insulin analogues may be grown as follows. The primary culture is prepared by inoculating two 2.8 liter (L) baffled Fernbach flasks containing 500 mL of BSGY media with a 2 mL Research Cell Bank of the relevant strain. After 48 hours of incubation, the cells are transferred to inoculate the bioreactor. The fermentation batch media contains: 40 g glycerol (Sigma Aldrich, St. Louis, Mo.), 18.2 g sorbitol (Acros Organics, Geel, Belgium), 2.3 g mono-basic potassium phosphate, (Fisher Scientific, Fair Lawn, N.J.) 11.9 g di-basic potassium phosphate (EMD, Gibbstown, N.J.), 10 g Yeast Extract (Sensient, Milwaukee, Wis.), 20 g Hy-Soy (Sheffield Bioscience, Norwich, N.Y.), 13.4 g YNB (BD, Franklin Lakes, N.J.), and 4×10⁻³ g biotin (Sigma-Aldrich, St. Louis, Mo.) per liter of medium.

Fermentations may be conducted in 15 L dished-bottom glass autoclavable and 40 L SIP bioreactors (8 L & 20 L starting volume respectively) (Applikon, Foster City, Calif.). The fermentations were run in a simple batch mode with the following conditions: temperature of 24±1° C.; pH of 6.0±0.1 maintained by the addition of 30% NH₄OH; airflow of approximately 0.7±0.1 vvm; dissolved oxygen of 20% of saturation is maintained by cascading feedback control of the agitation rate (from 250 to 800 rpm) followed by supplementation of pure oxygen to the sparged air stream up to 0.1 vvm. After the depletion of the initial charge of glycerol as seen by a sharp increase in dissolved oxygen concentration, a cell density of 100 +/−10 g/L (wet cell weight) is reached. At this point, the dissolved oxygen control is turned off and the agitation is fixed to a constant speed allowing for a constant oxygen uptake rate within the range of 35 to 90 mmol/L/hr. A 100% methanol feed solution is then initiated along with a shift in pH, from 6.0 to 5.2±0.1. Methanol is maintained in excess at a concentration of 0.15%±0.02% which is controlled by feedback from a Methanol Sensor (Raven Biotech Inc, Vancouver, British Columbia, Canada). The Methanol phase continues for 72±8 hours. At the end of the fermentation, the supernatant is obtained by centrifugation at 13,000×g for 30 minutes.

Protein expression for the transformed yeast strains disclosed herein may be carried out at in shake flasks at 24° C. with buffered glycerol-complex medium (BMGY) consisting of 1% yeast extract, 2% peptone, 100 mM potassium phosphate buffer pH 6.0, 1.34% yeast nitrogen base, 4×10⁻⁵% biotin, and 1% glycerol. The induction medium for protein expression is buffered methanol-complex medium (BMMY) consisting of 1% methanol instead of glycerol in BMGY. When desired to control or reduce O-glycosylation, a Pmt inhibitor such as Pmti-3 (5-[[3-(1-Phenyl-2-hydroxy)ethoxy)-4-(2-phenylethoxy)]phenyl]methylene]-4-oxo-2-thioxo-3-thiazolidineacetic Acid) (See Published International Application No. WO 2007061631) or Pmti-4 (Example 4 compound of U.S. Published Application No. 20110076721 having the structure

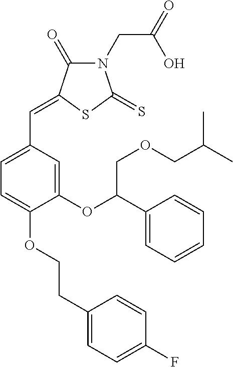 [SMILES: CC(C)COCC(Oc1cc(/C=C2\SC(=S)N(CC(=O)O)C2=O)ccc1OCCc1ccc(F)cc1)c1ccccc1]

in methanol is added to the growth medium to a final concentration of 18.3 μM at the time the induction medium was added. Cells are harvested and centrifuged at 2,000 rpm for five minutes.

SixFors Fermentor Screening Protocol followed the parameters shown in Table 2.

TABLE 2
SixFors Fermentor Parameters

| Parameter | Set-point | Actuated Element |
|---|---|---|
| pH | 6.5 ± 0.1 | 30% NH₄OH |
| Temperature | 24 ± 0.1 | Cooling Water & Heating Blanket |
| Dissolved O2 | n/a | Initial impeller speed of 550 rpm is |
| | | ramped to 1200 rpm over first 10 hr, then |
| | | fixed at 1200 rpm for remainder of run |

At time of about 18 hours post-inoculation, SixFors vessels containing 350 mL media A (See Table 3 below) plus 4% glycerol are inoculated with strain of interest. A small dose (0.3 mL of 0.2 mg/mL in 100% methanol) of Pmti-3 was added with inoculum. At time about 20 hour, a bolus of 17 mL 50% glycerol solution (Glycerol Fed-Batch Feed, See Table 4 below) plus a larger dose (0.3 mL of 4 mg/mL) of Pmti-3 or Pmti-4 is added per vessel. At about 26 hours, when the glycerol is consumed, as indicated by a positive spike in the dissolved oxygen (DO) concentration, a methanol feed (See Table 5 below) is initiated at 0.7 mL/hr continuously. At the same time, another dose of Pmti-3 or Pmti-4 (0.3 mL of 4 mg/mL stock) is added per vessel. At time about 48 hours, another dose (0.3 mL of 4 mg/mL) of Pmti-3 or Pmti-4 is added per vessel. Cultures are harvested and processed at time about 60 hours post-inoculation.

TABLE 3
Composition of Media A

| | | |
|---|---|---|
| Soytone L-1 | 20 | g/L |
| Yeast Extract | 10 | g/L |
| KH₂PO4 | 11.9 | g/L |
| K₂HPO₄ | 2.3 | g/L |
| Sorbitol | 18.2 | g/L |
| Glycerol | 40 | g/L |
| Antifoam Sigma 204 | 8 | drops/L |
| 10X YNB w/Ammonium Sulfate w/o | 100 | mL/L |
| Amino Acids (134 g/L) | | |
| 250X Biotin (0.4 g/L) | 10 | mL/L |
| 500X Chloramphenicol (50 g/L) | 2 | mL/L |
| 500X Kanamycin (50 g/L) | 2 | mL/L |

TABLE 4
Glycerol Fed-Batch Feed

| | | |
|---|---|---|
| Glycerol | 50 | % m/m |
| PTM1 Salts (see Table IV-E below) | 12.5 | mL/L |
| 250X Biotin (0.4 g/L) | 12.5 | mL/L |

TABLE 5
Methanol Feed

| | | |
|---|---|---|
| Methanol | 100 | % m/m |
| PTM1 Salts (See Table 6) | 12.5 | mL/L |
| 250X Biotin (0.4 g/L) | 12.5 | mL/L |

TABLE 6
PTM1 Salts

| | | |
|---|---|---|
| CuSO₄—5H₂O | 6 | g/L |
| NaI | 80 | mg/L |
| MnSO₄—7H₂O | 3 | g/L |
| NaMoO₄—2H₂O | 200 | mg/L |
| H₃BO₃ | 20 | mg/L |
| CoCl₂—6H₂O | 500 | mg/L |
| ZnCl₂ | 20 | g/L |
| FeSO₄—7H₂O | 65 | g/L |
| Biotin | 200 | mg/L |
| H₂SO₄ (98%) | 5 | mL/L |

### Example 8

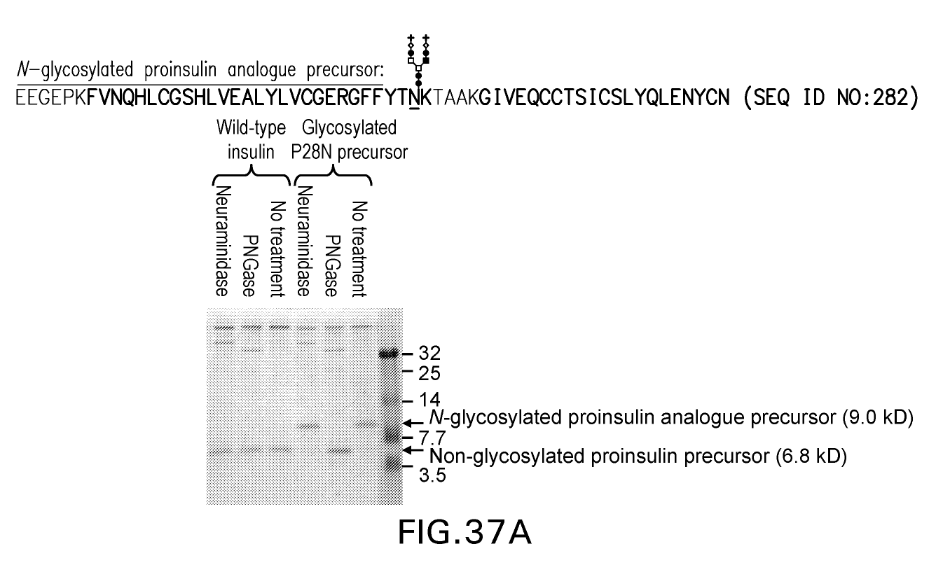

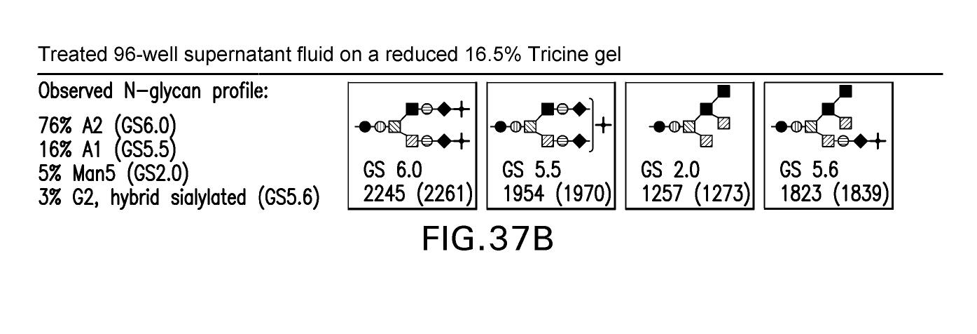

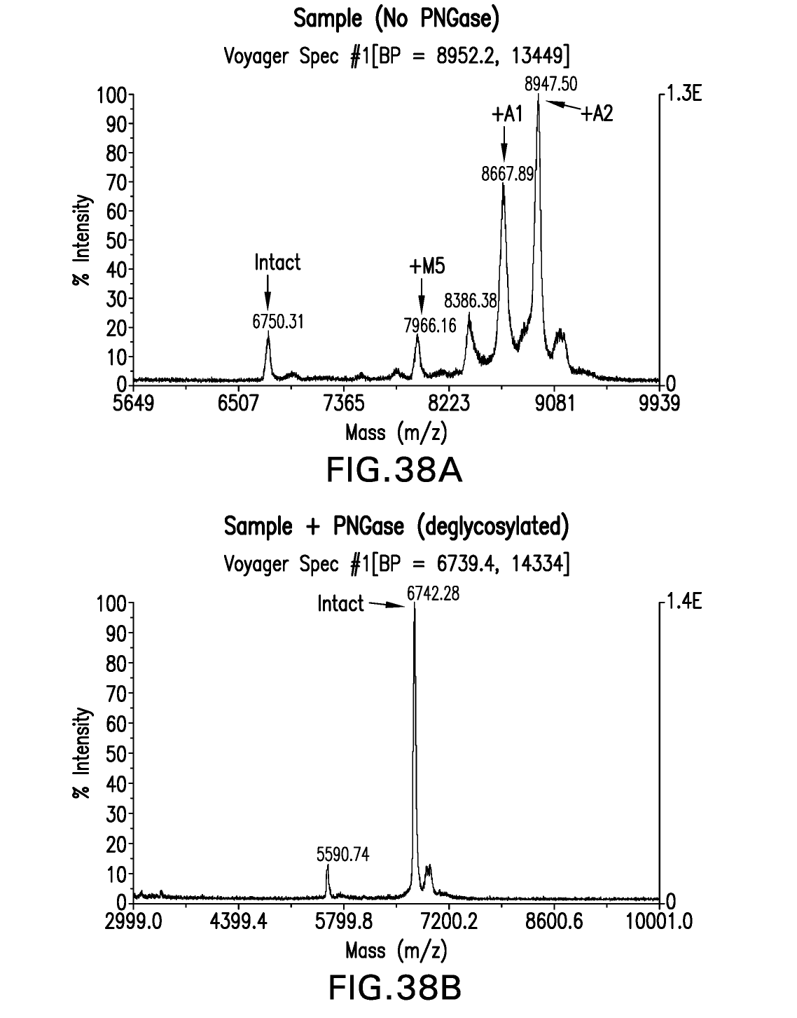

In this example, N-glycosylated insulin analogue precursors extracted from culture medium used to grow strain YGLY21058 were analyzed for N-linked glycosylation. The analogues are single-chain molecules having the amino acid sequence shown in SEQ ID NO:36. Aliquots of the culture medium were treated with PNGase or neuraminadase and the treated samples resolved on a reduced 16.5% TRICINE polyacrylamide gel along with an untreated aliquot as a control. FIG. 37A and FIG. 37B shows that the insulin analogue precursors were N-glycosylated. The N-glycans released by PNGase digestion were analyzed by positive and negative ion MALDI-TOF and the results are shown in FIG. 38A and FIG. 38B. The observed N-glycan composition of the insulin analogue precursors was about 75% A2 (bisialylated), about 16% was A1 (monosialylated), and about 5% was hybrid Man₅ as shown in FIG. 37A. FIG. 37B also shows the structure of the predominant insulin precursor species. In vitro processing of the N-glycosylated insulin analogue precursors would produce an N-glycosylated insulin analogue composition wherein the predominant N-glycan was bi-sialylated. The expected N-glycan composition would be expected to be about a 75:16:5:3 mol % ratio of NANA₂Gal₂GlcNAc₂Man₃GlcNAc₂ to NANAGal₂GlcNAc₂Man₃GlcNAc₂ to Man₅GlcNAc₂ to NANAGalGlcNAcMan₅GlcNAc₂.

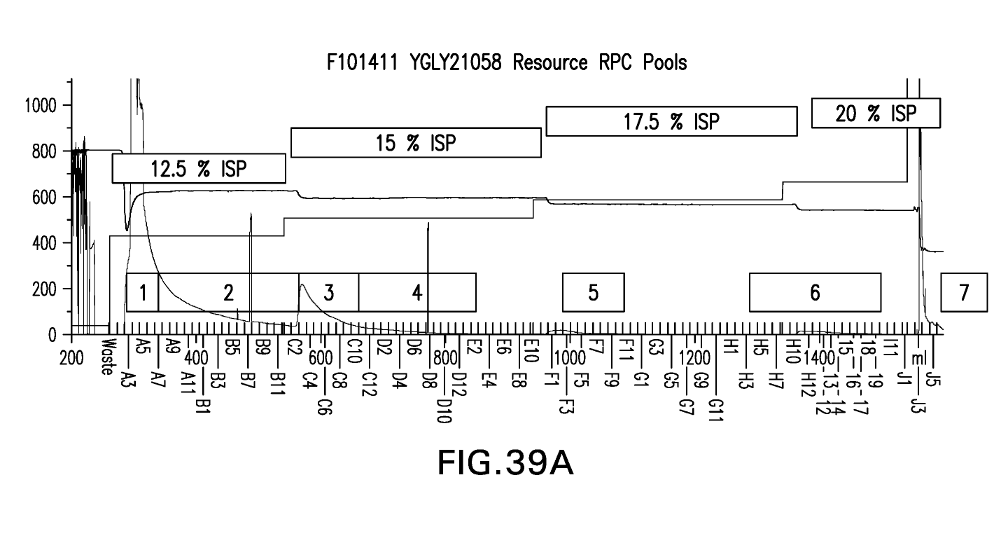

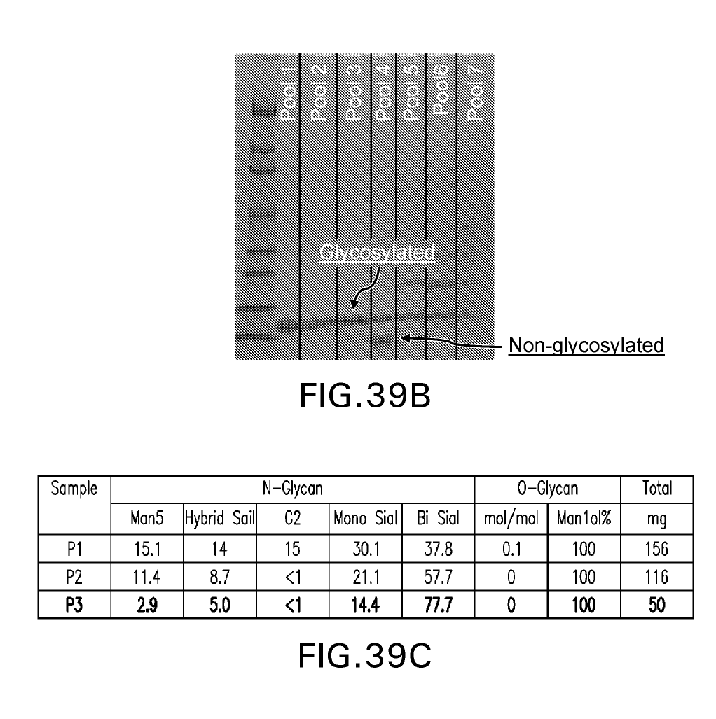

To purify the N-glycosylated insulin analogue precursors, supernatant medium was clarified by centrifugation for 15 min at 13,000 g in a Sorvall Evolution RC (kendo, Asheville, N.C.), followed by pH adjustment to 4.5 and filtered using a Sartopore 2 0.2 μm (Sartorius Biotech Inc). The filtrate was loaded to a Capto MIVIC column, a multimodal cation exchanger chromatography resin (GE Healthcare, Piscataway, N.J.) adjusted to the same pH. The pool obtained after elution at pH 7 was collected and loaded into a RESOURCE RPC column (Amersham Biosciences, Piscataway, N.J.), a reverse-phase column chromatography packed with SOURCE 15RPC, a polymeric, reversed-phase chromatography medium based on rigid, monodisperse 15 μm beads made of polystyrene/divinylbenzene. The resin was equilibrated at pH 3.5 and eluted using step elution from 12.5% to 20% 2-propanol at the same pH. The fractions were collected and pooled into seven groups as shown in FIG. 39A, FIG. 39B, and FIG. 39C. The seven groups were electrophoresed on a reduced 16.5% TRICINE polyacrylamide gel. To quantify the relative amount of each glycoform, the N-glycosidase F released glycans were labeled with 2-aminobenzidine (2-AB) and analyzed by HPLC as described in Choi et al., Proc. Natl. Acad. Sci. USA 100: 5022-5027 (2003) and Hamilton et al., Science 313: 1441-1443 (2006).

The following assay may be used to detect total sialic acid content on glycoproteins as a ratio of moles sialic acid/mole protein. Sialic acid is released from glycoprotein samples by acid hydrolysis and analyzed by HPAEC-PAD using the following method: About 10-15 μg of protein sample are buffer-exchanged into phosphate buffered saline. Four hundred μL of 0.1M hydrochloric acid is added, and the sample heated at 80° C. for 1 hour. After drying in a SpeedVac (Savant), the samples are reconstituted with 500 μL of water. One hundred μL is then subjected to HPAEC-PAD analysis. The yield and N-glycan composition of the N-glycosylated insulin analogue precursor pools 1-3 was also determined with results shown in FIG. 39A.

The pools were selected base on N-glycan composition for the enzymatic steps described below to produce compositions of N-glycosylated insulin analogue precursor having A2, G2, G0, or G-2 N-glycans. These N-glycans were generated on the N-glycosylated insulin analogue precursor analogue by consecutive enzymatic digestions. The enzymatic reactions conditions were used as recommended by the manufacturer. N-glycosylated insulin analogue precursor having A2 N-glycans were digested with acetyl-neuraminyl hydrolase (Sialidase, Neuraminidase) (New England BioLabs, Inc) to produce N-glycosylated insulin analogue precursors having G2 N-glycans. N-glycosylated insulin analogue precursors having G2 N-glycans were digested with β1-4 Galactosidase (New England BioLabs, Inc) to produce N-glycosylated insulin analogue precursors having G0 N-glycans. N-glycosylated insulin analogue precursor G0 was digested with β-N-acetylglucosaminidase (hexosaminidase) (New England BioLabs, Inc) to produce N-glycosylated insulin analogue precursor having G-2 N-glycans. The last enzymatic step applied to all the above species was to digest the N-glycosylated insulin analogue precursor to completion using endoproteinase Lys-C(Roche) to produce an N-glycosylated insulin heterodimer having a native human insulin A-chain peptide and a des(B30) B:P28N B-chain peptide wherein the Asn at position 28 is attached to an A2 N-glycan (GS6.0), a G2 N-glycan (GS5.0), a G0 N-glycan (GS4.0), or a G-2 N-glycan (GS2.1). The amino acid sequences of the B-chain of the various analogues are shown by SEQ ID NOs. 294, 295, 296, and 297, respectively.

Following the enzymatic digestions, the resulting N-glycosylated des(B30) B:P28N insulin heterodimers were purified using SOURCE 15RPC as described above. The final pool was formulated in 25 mM Sodium Phosphate dibasic (Anhydrous), 10 mM NaCl, 1.6% glycerol pH 7.4. This final formulated protein was used for all the in vitro and in vivo studies. In parallel, commercial NOVOLIN (Novo Nordisk) was digested using endoproteinase Lys-C(Roche) to produce a des(B30) form to use as a control. Purification and formulation was performed as described above.

### Example 9

To study the glucose responsiveness of the GS2.1 and GS5.0 insulin analogues, C57BL/6 mice at 12 weeks of age were fasted two hours before dosed with GS2.1 or GS5.0 by s.c injection. At the same time, animals received i.p. administration of α-methylmannose solution (21.5% w/v in saline, 10 ml/kg) or vehicle. At high concentrations, α-methylmannose is known to competitively inhibit interactions between c-type lectins and glycoproteins, especially those terminating in mannose, GlcNAc, or fucose residues. Blood glucose was measured using a glucometer (OneTouch Ultra LifeScan; Milpitas, Calif.) at time 0 and then 30, 60, 90, and 120 minutes post injection. Glucose Area-Over-the-Curve (AOC) was calculated using values normalized to glucose of time 0 (as 100%).

As shown in FIG. 40A, FIG. 40B, FIG. 40C, and FIG. 40D, GS5.0, which contains terminal galactose, dosed at 18 nmol/kg lowered glucose during 120 min study period. Injection of α-methylmannose had no detectable additional effect on glucose lowering induced by GS5.0. In contrast, GS2.1, which contains terminal mannose, lowered glucose when dosed alone but to a lesser extent compared to GS5.0. However, in the presence of α-methylmannose, GS2.1 lowered glucose with better or greater potency at 60 and 90 minutes than GS5.0. The percent glucose AOC in the presence and absence of α-methylmannose was significantly different for GS2.1 whereas no change was detected for GS5.0. Glucose is known to inhibit interactions between mannose-binding c-type lectins and glycoproteins, albeit with less potency than α-methylmannose. These data show that GS2.1 can lower glucose in a glucose responsive fashion, possibly mediated by mannose binding lectins such as mannose receptor.

### Example 10

This example shows the production of N-glycosylated proinsulin analogue precursors that contain zero, one, two, or three N-glycans. The N-glycans were either GS 1.0 (Man₍₈₋₁₂₎GlcNAc₂) or GS2.0 (Man₅GlcNAc₂).

Each of the expression vectors shown in Table 1 in Example 1 was separately transformed into strain YGLY26268. Strain YGLY26268 is a GFI1.0 strain that lacks alpha-1,6-mannosyltransferase activity but produces glycoproteins that have high mannose N-glycans (Man₍₈₋₁₂₎GlcNAc₂) with high N-glycosylation site occupancy due to the presence of the LmSTT3D gene.

Three clones from each transfection were cultivated in Micro24 reactors (Pall Corporation) and recombinant protein was induced upon addition of methanol. Resulting culture supernatant fluids were isolated from the three different clones from each transformation and analyzed for protein expression by gel electrophoresis on a reduced 4-20% Tris-HCl SDS polyacrylamide gel and the proteins visualized with coomassie blue staining. Two control strains, designated YGLY26580 and YGLY26734, were generated in previous transformations and included in the experimental run.

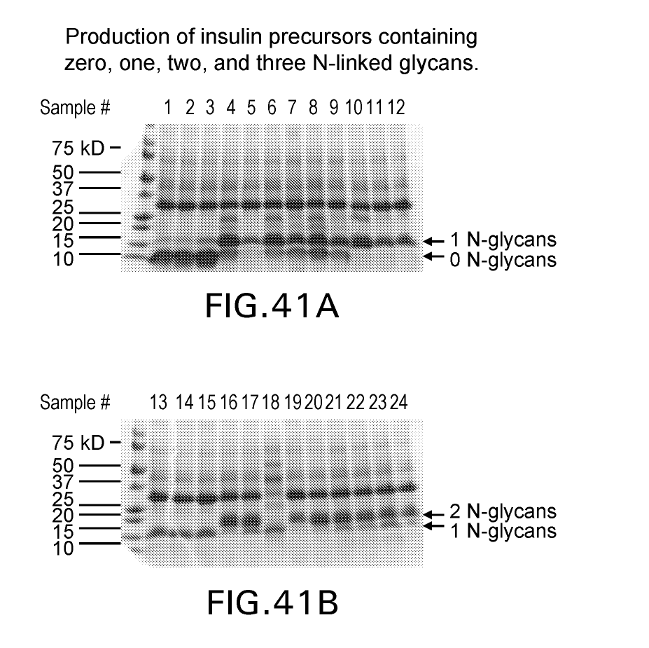

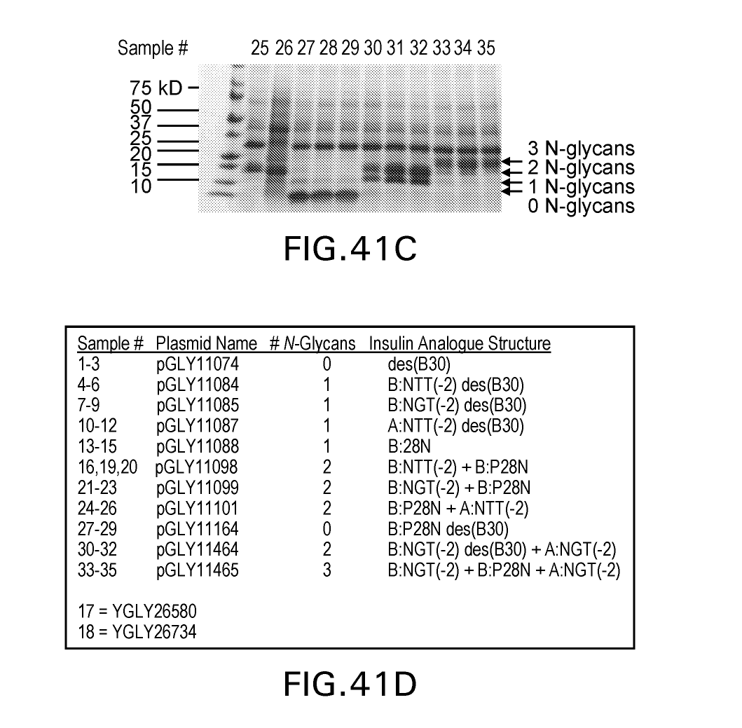

The results of the gel electrophoresis are shown in FIG. 41A, FIG. 41B, FIG. 41C, and FIG. 41D. The results show that proinsulin precursor analogues with N-linked glycosylation sites were N-glycosylated with predominantly Man₍₈₋₁₂₎GlcNAc₂ N-glycans and migrated with protein molecular weights consistent with the predicted number of N-glycans, each N-glycan having a molecular weight of about 1720 Daltons. The proinsulin precursor analogue encoded by pGLY11164 was not glycosylated because while it contained an asparagine residue at position B28, it lacked a threonine residue at position B30 and thus, lacked a complete N-linked glycosylation motif.

Control strain YGLY26734 produced a proinsulin analogue precursor which in lane 18 of the gel shown in FIG. 41B appears to migrate at a position corresponding to analogues containing one N-glycosylation site (e.g., 13-14). However, the proinsulin analogue precursor is glycosylated at both positions. The shift in mobility is due to the decrease in size of the N-glycans compared to the N-glycans for the proinsulin analogue precursors produced in the GFI1.0 strains. The high mannose N-glycans have an average molecular weight of about 1720 Daltons whereas the Man₅GlcNAc₂ N-glycans have a molecular weight of about 1257 Daltons, a difference of about 463 Daltons. Since there are two N-glycosylation sites, the total decrease in size is about 926 Daltons. This difference in molecular weight between the proinsulin analogue precursors having high mannose N-glycans verses Man₅GlcNAc₂ N-glycans affects the mobility of the respective proinsulin analogue precursors as shown in the gel.

### Example 11

This example describes construction of strain YGLY26268 of Example 10. Strain YGLY26268 is capable of producing glycoproteins with GS 1.0 (Man₍₈₋₁₂₎GlcNAc₂) N-glycans and includes the LmSTT3D gene, which has been shown in PCT/US2011/25878 to effect an increase N-glycosylation site occupancy compared to strains that lack the 1mSTT3D gene.

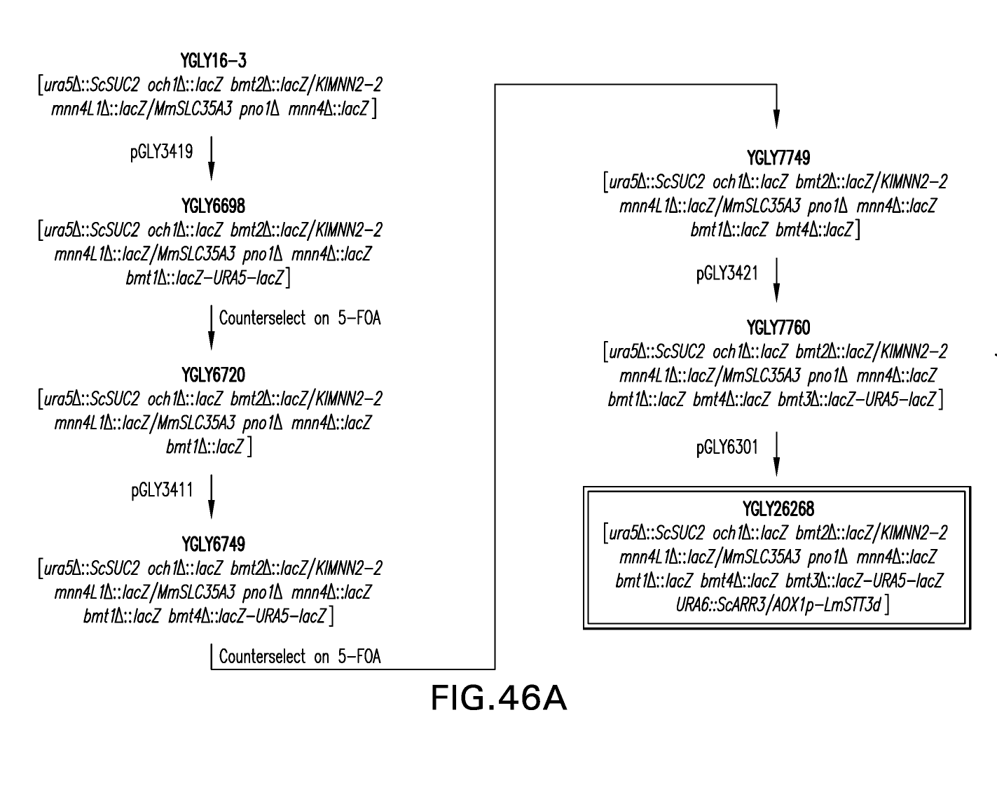

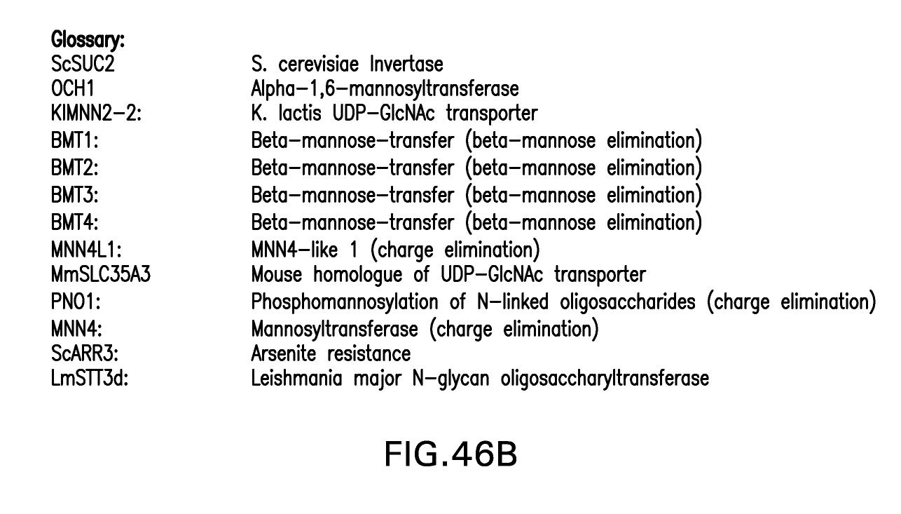

Construction of strain YGLY26268 is shown in FIG. 46. Briefly, strain YGLY16-3 was transformed with plasmid pGLY3419 as described previously to produce a number of strains of which YGL6698 and YGLY6697 were selected. The two selected strains were counterselected in the presence of 5-fluoroorotic acid (5-FOA) to produce a number of strains of which YGLY6720 and YGLY6719 were selected.

Strains YGLY6720 and YGLY6719 were each transfected with plasmid pGLY3411 as described previously to produce a number of strains of YGLY6749 and YGLY6743 were selected. The two selected strains were counterselected in the presence of 5-fluoroorotic acid (5-FOA) to produce a number of strains of which YGLY7749 and YGLY6773 were selected.

Strains YGLY7749 and YGLY6773 were each transfected with plasmid pGLY3421 as described previously to produce a number of strains of YGLY7760 and YGLY7754 were selected.

Plasmid pGLY6301 is a roll-in integration plasmid that targets the URA6 locus in P. pastoris. The expression cassette encoding the LmSTT3D comprises a nucleic acid molecule encoding the LmSTT3D ORF codon-optimized for effective expression in P. pastoris operably linked at the 5′ end to a nucleic acid molecule that has the inducible P. pastoris AOX1 promoter sequence (SEQ ID NO:118) and at the 3′ end to a nucleic acid molecule that has the S. cerevisiae CYC transcription termination sequence (SEQ ID NO:58). For selecting transformants, the plasmid comprises an expression cassette encoding the S. cerevisiae ARR3 ORF in which the nucleic acid molecule encoding the ORF (SEQ ID NO:255) is operably linked at the 5′ end to a nucleic acid molecule having the P. pastoris RPL10 promoter sequence (SEQ ID NO:257) and at the 3′ end to a nucleic acid molecule having the S. cerevisiae CYC transcription termination sequence (SEQ ID NO:58). The plasmid further includes a nucleic acid molecule for targeting the URA6 locus (SEQ ID NO:256). Plasmid pGLY6301 was constructed by cloning the DNA fragment encoding the codon-optimized LmSTT3D ORF (pGLY6287) flanked by an EcoRI site at the 5′ end and an FseI site at the 3′ end into plasmid pGFI30t, which had been digested with EcoRI and FseI.

Strain YGLY7760 was transfected with pGLY6301 as described previously to produce a number of strains of which strain YGLY26268 was selected. Strain YGLY26268 was transformed with alternate insulin expression plasmids as listed in Table 1 in Example 1 above. All insulin expression plasmids from Table 1 were generated through cloning of the insulin precursor gene using restriction sites MlyI and FseI into plasmid pGLY9316 (FIG. 47) and has open reading frames as shown in SEQ ID NO:126 (pGLY11074), SEQ ID NO: 128 (pGLY11084), SEQ ID NO: 130 (pGLY11085), SEQ ID NO: 132 (pGLY11087), SEQ ID NO: 134 (pGLY11088), SEQ ID NO: 136 (pGLY11098), SEQ ID NO: 138 (pGLY11099), SEQ ID NO: 140 (pGLY11101), SEQ ID NO: 142 (pGLY11164), SEQ ID NO: 144 (pGLY11464), and SEQ ID NO: 146 (pGLY11465). Clones derived from YGLY26268 are GFI1.0 strains that are capable of producing glycoproteins that have predominantly Man₍₈₋₁₂₎GlcNAc₂ structures.

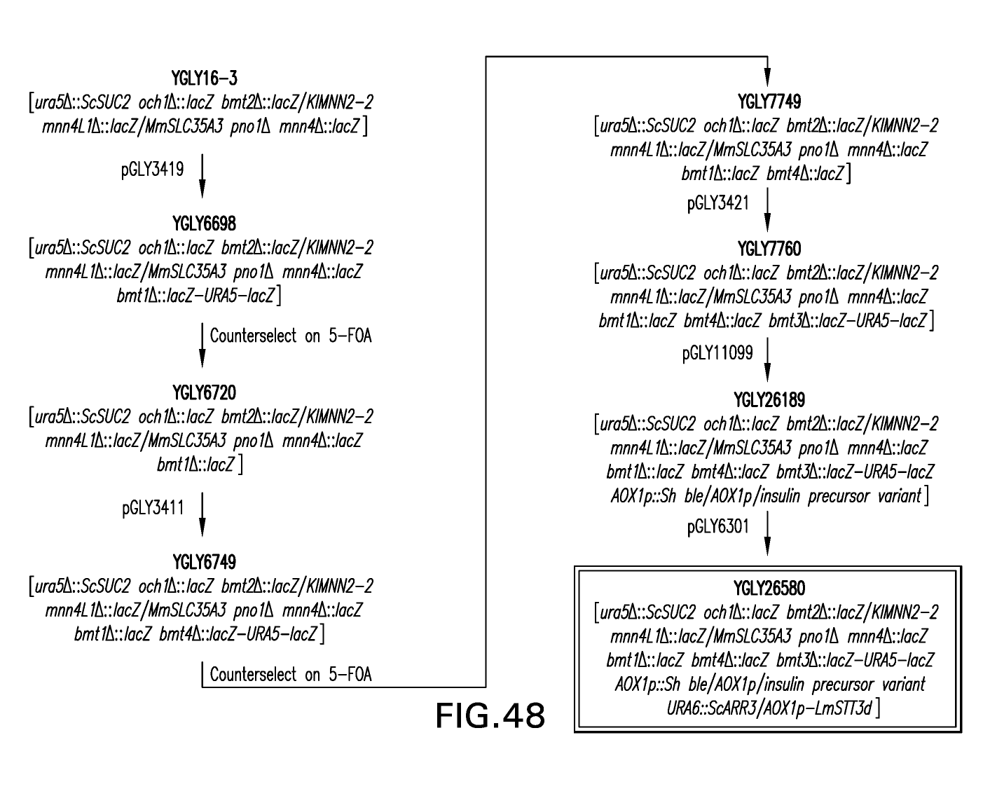

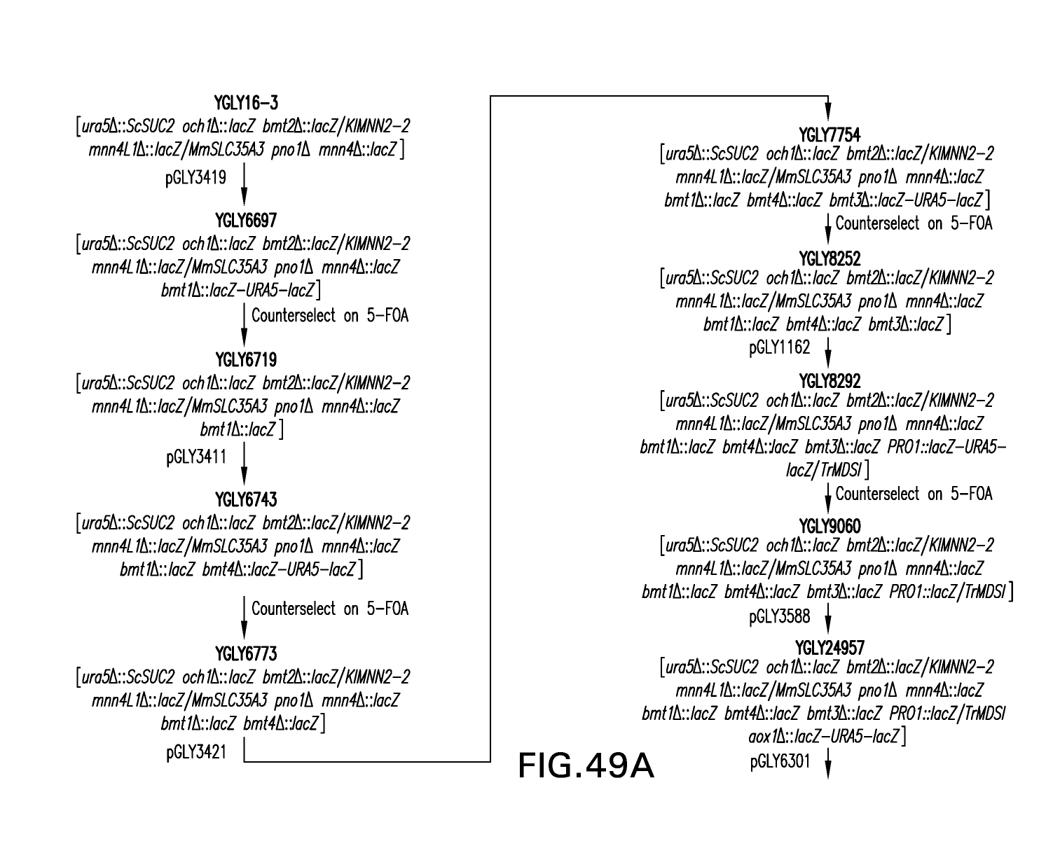

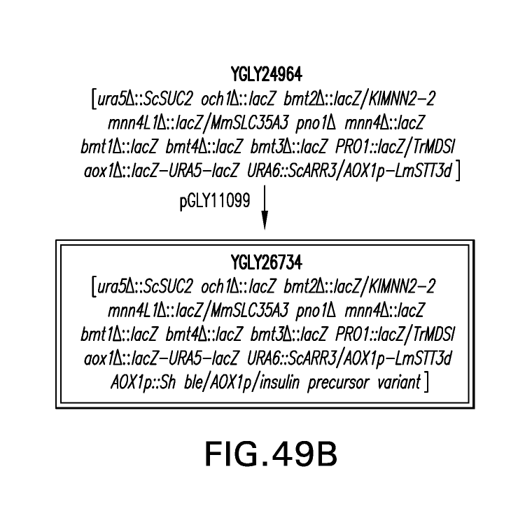

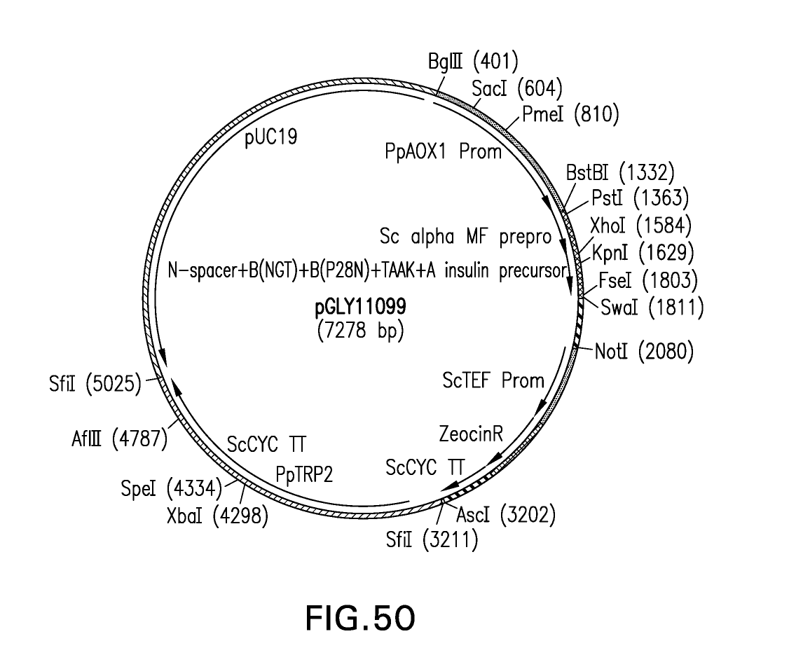

The control strains in this experiment, YGLY26580 and YGLY26734 produce an N-glycosylated insulin analogue precursor with the amino acid sequence shown in SEQ ID NO:156 from plasmid pGLY11099. The N-glycosylated insulin analogue precursor has two N-glycans: one at position B(−2) and one at position B28. While both YGLY26580 and YGLY26734 contain the insulin expression plasmid pGLY11099, YGLY26580 is a GFI1.0 strain that produces glycoproteins with predominantly Man₍₈₋₁₂₎GlcNAc₂ N-glycan structures while YGLY26734 is a GFI2.0 strain that produces glycoproteins with predominantly a Man₅GlcNAc₂ N-glycan structure. The construction of strain YGLY26580 is shown in FIG. 48 and described in Example 12 while the construction of strain YGLY26734 is shown in FIGS. 49A-49B and described in Example 13. The map of plasmid pGLY11099 is shown in FIG. 50.

### Example 12

Construction of strain YGLY26580 is shown in FIG. 48. The strain is a control strain that produces the insulin analogue encoded by pGLY11099 with GS1.0 (Man₍₈₋₁₂₎GlcNAc₂) N-glycans and includes the LmSTT3D gene.

Briefly, strain YGLY7760 was transfected with plasmid pGLY11099 to produce a number of strains of which YGLY26189 was selected. Plasmid pGLY11099 (FIG. 50) encodes an insulin analogue comprising an N-glycosylation site at position B-2 and position B28. The amino acid sequence of the proinsulin precursor analogue encoded by the plasmid is shown in SEQ ID NO:156.

Strain YGLY26189 was transfected with pGLY6301 as described previously to produce a number of strains of which strain YGLY26580 was selected.

### Example 13

Construction of control strain YGLY26734 is shown in FIG. 49. The strain is a control strain that produces the insulin analogue precursor encoded by pGLY11099 with GS2.0 (Man₅GlcNAc₂) N-glycans at position B(−2) and position B28 and includes the LmSTT3D gene. The glycosylated insulin analogue precursor can be processed in vitro to glycosylated insulin analog 200-2-B. 200-2-B is a heterodimer comprising a native insulin A-chain and a B-chain (des(B30)) having the amino acid sequence N*GTFVNQHLCGSHLVEALYLVCGERGFFYTN*K (SEQ ID NO:293) wherein the Asn residues N* at positions 1 and 31 (B-2 & B28) are each covalently linked in a β1 linkage to a Man₅GlcNAc₂ N-glycan. Construction of strain YGLY26734 is as follows.

Strain YGLY7754 was counterselected in the presence of 5-fluoroorotic acid (5-FOA) to produce a number of strains of which YGLY8252 was selected.

Plasmid pGLY1162 (FIG. 51) is a KINKO integration vector that targets the PRO1 locus without disrupting expression of the locus and contains expression cassettes encoding the T. reesei α-1,2-mannosidase catalytic domain fused at the N-terminus to S. cerevisiae αMATpre signal peptide (aMATTrMan) to target the chimeric protein to the secretory pathway and secretion from the cell. The expression cassette encoding the aMATTrMan comprises a nucleic acid molecule encoding the T. reesei catalytic domain fused at the 5′ end to a nucleic acid molecule encoding the S. cerevisiae αMATpre signal peptide, which is operably linked at the 5′ end to a nucleic acid molecule comprising the P. pastoris AOX1 promoter and at the 3′ end to a nucleic acid molecule comprising the S. cerevisiae CYC transcription termination sequence. The cassette is flanked on one side by a nucleic acid molecule comprising a nucleotide sequence from the 5′ region and complete ORF of the PRO1 gene (SEQ ID NO:119) followed by a P. pastoris ALG3 termination sequence and on the other side by a nucleic acid molecule comprising a nucleotide sequence from the 3′ region of the PRO1 gene (SEQ ID NO:120). The plasmid contains a nucleic acid molecule comprising the P. pastoris URA5 gene or transcription unit flanked by nucleic acid molecules comprising lacZ repeats. Plasmid pGLY1162 was transformed into strains YGLY8252 to produce a number of strains of which strain YGLY8292 was selected from the strains produced. Strain YGLY8292 was counterselected in the presence of 5-fluoroorotic acid (5-FOA) to produce a number of strains of which YGLY9060 was selected.

Strain YGLY9060 was transformed with plasmid pGLY3588 described previously to produce a number of strains of which strain YGLY24957 was selected. Strain YGLY24957 was transformed with plasmid pGLY6301 to produce a number of strains of which YGLY24964 was selected. Strain YGLY24964 was transformed with plasmid pGLY11099 to produce a number of strains of which strain YGLY26734 was selected.

Following the fermentation of strain YGLY26734, the insulin analogue precursor was purified from cell-free fermentation supernatant and processed with the LysC endoproteinase to produce the des(B30) heterodimer 200-2-B for in vitro and in vivo testing as described in Example 15.

### Example 14

This example describes construction of strain YGLY29365. Strain YGLY29365 is capable of producing a glycosylated insulin analogue precursor with GS2.1 (Man₃GlcNAc₂) N-glycans at position B(−2) and position B28. The glycosylated insulin precursor can be processed in vitro to glycosylated insulin analog 210-2-B. 210-B-2 is a heterodimer comprising a native insulin A-chain and a B-chain (des(B30)) having the amino acid sequence N*GTFVNQHLCGSHLVEALYLVCGERGFFYTN*K (SEQ ID NO:292) wherein the Asn residues N* at positions 1 and 31 (B-2 & B28) are each covalently linked in a β1 linkage to a Man₃GlcNAc₂ (paucimannose) N-glycan.

The construction of strain YGLY29365 is the product of numerous genetic modifications beginning with the strain YGLY9060 shown in FIG. 49A and described in Example 13.

Strain YGLY9060 was transformed with plasmid pGLY7140, a knock-out vector that targets the YOS9 locus and contains a nucleic acid molecule comprising the P. pastoris URA5 gene (SEQ ID NO:49) or transcription unit flanked by nucleic acid molecules comprising lacZ repeats (SEQ ID NO:50) which in turn is flanked on one side by a nucleic acid molecule comprising a nucleotide sequence from the 5′ region of the YOS9 gene (SEQ ID NO:306) and on the other side by a nucleic acid molecule comprising a nucleotide sequence from the 3′ region of the YOS9 gene (SEQ ID NO:307). The Yos9p has been implicated in the ER-associated degradation (ERAD) pathway (See Kim et al., Mol. Cell. 16: 741-751 (2005): deleting the YOS9 gene may improve yield of glycosylated protein. Plasmid pGLY7140 was linearized with SfiI and the linearized plasmid transformed into strain YGLY9060 to produce a number of strains in which the URA5 gene flanked by the lacZ repeats has been inserted into the YOS9 locus by double-crossover homologous recombination. Strain YGLY23328 was selected from the strains produced. The strain YGLY23328 was counterselected in the presence of 5-FOA to produce strain YGLY23360 in which the URA5 gene has been lost and only the lacZ repeats remain.

Strain YGLY24542 was generated by transforming plasmid pGLY5508, a knock-out vector that targets the ALG3 locus and contains a nucleic acid molecule comprising the P. pastoris URA5 gene or transcription unit flanked by nucleic acid molecules comprising lacZ repeats which in turn is flanked on one side by a nucleic acid molecule comprising a nucleotide sequence from the 5′ region of the ALG3 gene (SEQ ID NO:308) and on the other side by a nucleic acid molecule comprising a nucleotide sequence from the 3′ region of the ALG3 gene (SEQ ID NO:309). Plasmid pGLY5508 was linearized with SfiI and the linearized plasmid transformed into strain YGLY23360 to produce a number of strains in which the URA5 gene flanked by the lacZ repeats has been inserted into the ALG3 locus by double-crossover homologous recombination. Strain YGLY24542 was selected from the strains produced.

Plasmid pGLY10153 is a roll-in integration plasmid that targets the URA6 locus in P. pastoris and encodes the LmSTT3A, LmSTT3B, and LmSTT3D ORFs. Overexpressing the LmSTT3 proteins may enhance N-glycosylation site occupancy of the insulin analogues. The expression cassette encoding the LmSTT3A comprises a nucleic acid molecule encoding the LmSTT3D ORF codon-optimized for effective expression in P. pastoris (SEQ ID NO:310) operably linked at the 5′ end to a nucleic acid molecule that has the inducible P. pastoris AOX1 promoter sequence and at the 3′ end to a nucleic acid molecule that has the S. cerevisiae CYC transcription termination sequence. The expression cassette encoding the LmSTT3B comprises a nucleic acid molecule encoding the LmSTT3B ORF codon-optimized for effective expression in P. pastoris (SEQ ID NO:311) operably linked at the 5′ end to a nucleic acid molecule that has the inducible P. pastoris AOX1 promoter sequence and at the 3′ end to a nucleic acid molecule that has the S. cerevisiae CYC transcription termination sequence. The expression cassette encoding the LmSTT3D comprises a nucleic acid molecule encoding the LmSTT3D ORF codon-optimized for effective expression in P. pastoris (SEQ ID NO:121) operably linked at the 5′ end to a nucleic acid molecule that has the inducible P. pastoris AOX1 promoter sequence and at the 3′ end to a nucleic acid molecule that has the S. cerevisiae CYC transcription termination sequence. For selecting transformants, the plasmid comprises an expression cassette encoding the S. cerevisiae ARR3 ORF in which the nucleic acid molecule encoding the ORF is operably linked at the 5′ end to a nucleic acid molecule having the P. pastoris RPL10 promoter sequence and at the 3′ end to a nucleic acid molecule having the S. cerevisiae CYC transcription termination sequence. Plasmid pGLY10153 was transformed into strain YGLY24542 to produce a number of strains of which strain YGLY24561 was selected. Strain YGLY24561 was counterselected in the presence of 5-FOA to produce strain YGLY24586 in which the URA5 gene has been lost and only the lacZ repeats remain.

Strain YGLY24586 was transformed with plasmid pGLY5933, which disrupts the ATT1 gene. Disruption of the ATT1 gene may provide improve cell fitness during fermentation. The salient features of the plasmid is that it comprises the URA5 expression cassette described above flanked on one end with a nucleic acid molecule comprising the 5′ or upstream region of the ATT1 gene (SEQ ID NO:312) and the other end with a nucleic acid molecule encoding the 3′ or downstream region of the ATT1 gene (SEQ ID NO:313). YGLY24586 was transformed with plasmid pGLY5933 resulted in a number of strains of which strain YGLY27303 was selected. Strain YGLY27303 was transformed with plasmid pGLY11099 (FIG. 50) to produce a number strains of which strain YGLY28137 was selected.

Plasmid pGLY12027 is a roll-in integration plasmid that targets the URA6 locus in P. pastoris and encodes the murine endomannosidase ORF. The expression cassette encoding the full-length murine endomannosidase comprises a nucleic acid molecule encoding full-length murine endomannosidase ORF codon-optimized for effective expression in P. pastoris (SEQ ID NO:314) operably linked at the 5′ end to a nucleic acid molecule that has the inducible P. pastoris AOX1 promoter sequence and at the 3′ end to a transcription termination sequence, for example the Pichia pastoris AOX1 transcription termination sequence (SEQ ID NO:315). For selecting transformants, the plasmid includes the NAT^{R} expression cassette (SEQ ID NO:108) operably regulated to the Ashbya gossypii TEF1 promoter (SEQ ID NO:109) and A. gossypii TEF1 termination sequence (SEQ ID NO:110). The plasmid further includes a nucleic acid molecule as described previously for targeting the URA6 locus. Strain YGLY28137 was transformed with plasmid pGLY12027 to generate a number of strains of which strain YGLY29365 was selected.

Following the fermentation of strain YGLY29365, the insulin analogue precursor was purified from cell-free fermentation supernatant and processed with the LysC endoproteinase to produce the des(B30) heterodimer 210-2-B for in vitro and in vivo testing as described in Example 15.

### Example 15

This example shows two N-glycosylated insulin analogues that exhibit glucose-responsive properties. The first insulin analogue is denoted 210-2-B and is a heterodimer comprising a native insulin A-chain and a B-chain (des(B30)) having the amino acid sequence N*GTFVNQHLCGSHLVEALYLVCGERGFFYTN*K (SEQ ID NO:292) wherein the Asn residues N* at positions 1 and 31 (B-2 & B28) are each covalently linked in a β1 linkage to a Man₃GlcNAc₂ (paucimannose) N-glycan. The second analogue is denoted 200-2-B is a heterodimer comprising a native insulin A-chain and a B-chain (des(B30)) having the amino acid sequence N*GTFVNQHLCGSHLVEALYLVCGERGFFYTN*K (SEQ ID NO:293) wherein the Asn residues N* at positions 1 and 31 (B-2 & B28) are each covalently linked in a β1 linkage to a Man₅GlcNAc₂ N-glycan. The N-glycosylated insulin analogues are B:NGT at N-terminus, B:P28N, des(B30).

To assess the activity of these analogs, three in vitro assays were performed. Binding to the human insulin receptor isoform B (IR-b) was determined in a competition of the analog with radiolabeled human insulin to Chinese hamster ovary (CHO) cells over-expressing IR-b and presented as an IC50 value. Functional activation of IR-b was determined by assessing the phosphorylation of IR-b in Chinese hamster ovary (CHO) cells over-expressing IR-b and presented as an EC50 value. Binding to the human mannose receptor C type 1 (MRC1) was determined in a competition of the analog with europium-labeled mannose-BSA to the ectodomain of MRC1 in an ELISA assay and presented as an IC50 value. The in vitro properties of IR-b binding, IR-b phosphorylation, and MRC1 binding of the analogues compared to the binding of recombinant human insulin (RHI) are shown in Table 7.

TABLE 7

| | | Human IRb | Human MRC1 |
|---|---|---|---|
| | Human IRb Bound | Phosphorylation | Bound |
| Analogue | (nM) | (nM) | (nM) |
| 210-2-B | 0.81 | 0.79 | 0.714 |
| 200-2-B | 0.89 | 1.02 | 0.988 |
| RHI | 0.2 | 0.3 | >10000 |

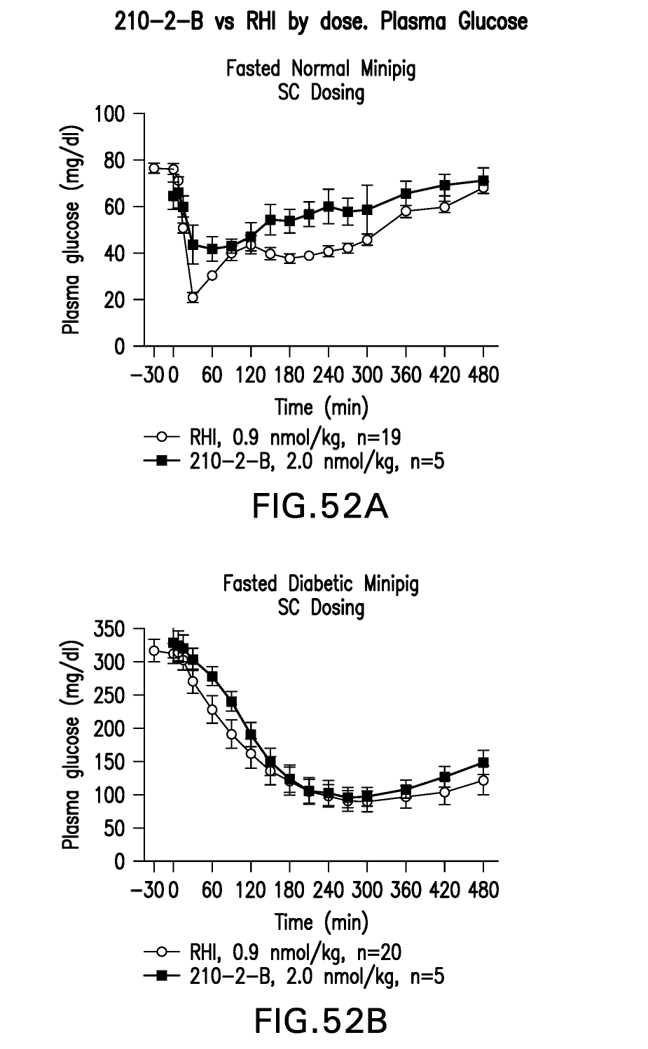

In vivo, binding of an insulin analog to MRC1 under euglycemic and hypoglycemic conditions may lead to an alternative route of insulin clearance not associated with a resulting lowering of blood glucose, whereas hyperglycemic conditions may enable glucose to compete for the binding of the analog to MRC1 and lead to higher rates of IR binding, clearance, and associated reduction in blood glucose. An insulin analog deficient in MRC1 binding, such as recombinant human insulin, may therefore be fully active under all blood glucose states with the potential to cause severe hypoglycemia. Therefore, the analogs 210-2-B and 200-2-B were tested in a Yucatan minipig model to assess glucose-responsiveness. Normal Yucatan minipigs were administered alloxan, allowed to recover, and given twice daily subcutaneous injections of NPH insulin in a model of type I diabetes. Five normal and five diabetic minipigs were fasted two hours before dosing with the insulin analogue by subcutaneous (s.c.) injection. Blood glucose was measured using a glucometer (e.g., OneTouch Ultra LifeScan; Milpitas, Calif.) at time 0 and 8, 15, 30, 60, 90, 120, 150, 180, 210, 240, 270, 300, 360, 420, and 480 minutes post injection. The results of one such experiment in fasted normal and diabetic minipigs are shown in FIGS. 52A to 55B.

FIG. 52A shows that N-glycosylated insulin analogue 210-2-B administered subcutaneously (s.c.) to the fasted diabetic minipig at 2.0 nmol/kg produces an effect on blood glucose levels over time that is equivalent to the effect of RHI has on blood glucose levels when administered subcutaneously (s.c.) to the fasted diabetic minipig at 0.9 nmol/kg.

FIG. 52B shows a comparison of the effect of N-glycosylated insulin analogue 210-2-B (paucimannose linked to Asn residues at B-2 and B28) versus recombinant human insulin (RHI) on blood glucose levels over time when administered subcutaneously (s.c.) to the fasted normal minipig. The figure shows that 210-2-B delivered at 2.0 nmol/kg causes less of a change in blood glucose levels that caused by RHI delivered at 0.9 nmol/kg. The figure also shows that the change in glucose levels observed for 210-2-B is less likely to result in severe hypoglycemia.

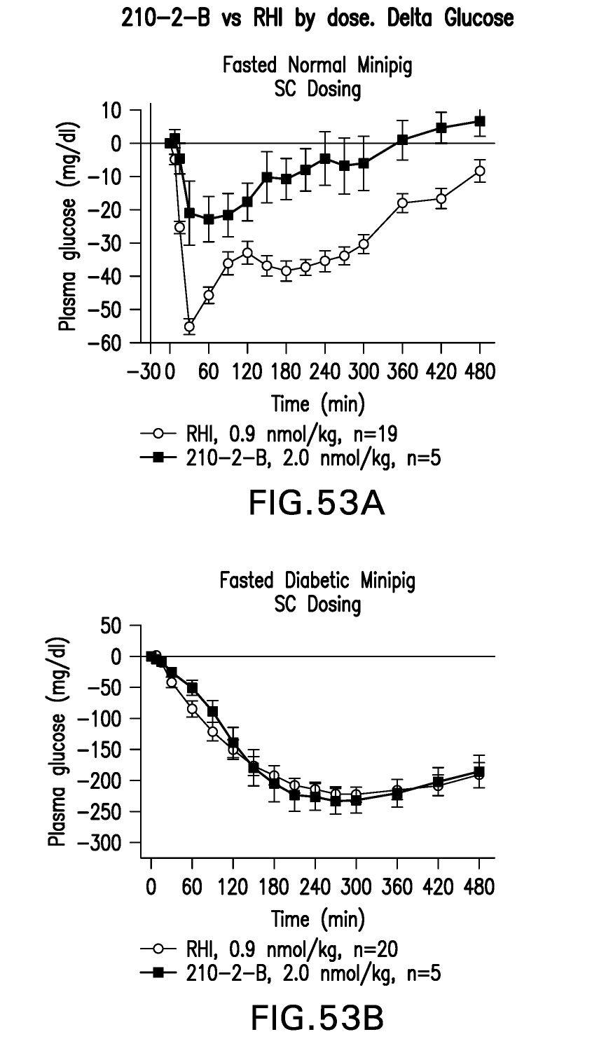

FIG. 53A shows the data shown in FIG. 52B replotted as change in blood glucose from baseline and FIG. 53B shows the data shown in FIG. 52A replotted as change in blood glucose from baseline. These Figures show that 210-2-B affects blood glucose levels in a glucose-responsive manner. FIG. 53B also shows that 210-2-B is controlling blood glucose levels in the fasted diabetic minipig.

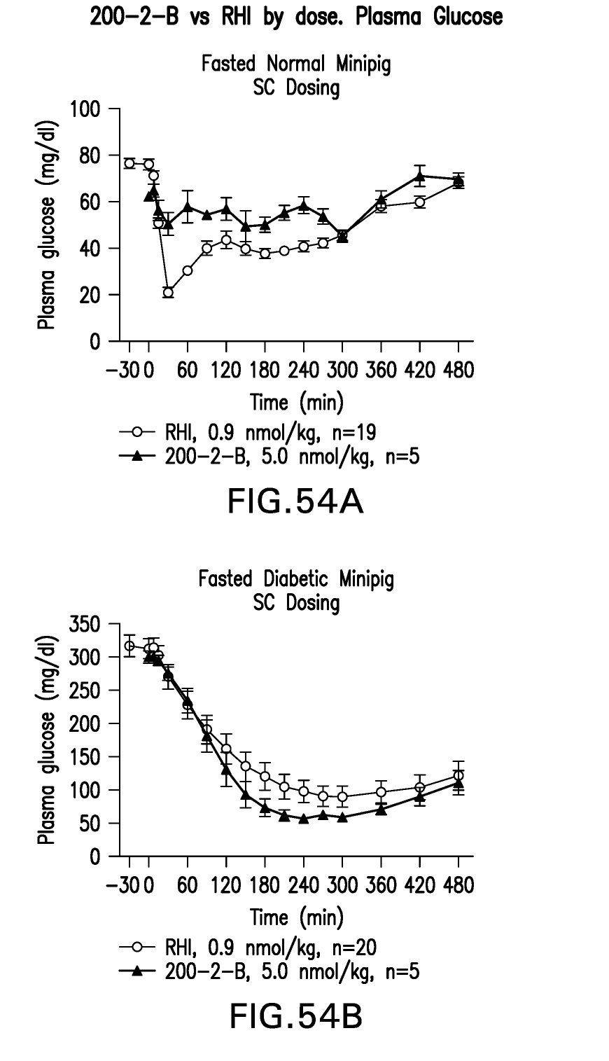

FIG. 54A shows the dosage of N-glycosylated insulin analogue 200-2-B that when administered subcutaneously (s.c.) to the fasted diabetic minipig produces an effect on blood glucose levels over time that is equivalent to the effect of RHI has on blood glucose levels when administered subcutaneously (s.c.) to the fasted diabetic minipig. The Figure shows that 5 nmol/kg of 200-2-B is equivalent to 0.9 nmol/kg of RHI in blood glucose lowering effect in fasted diabetic minipigs.

FIG. 54B shows a comparison of the effect of N-glycosylated insulin analogue 200-2-B (Man₅GlcNAc₂ linked to Asn residues at B-2 and B28) versus recombinant human insulin (RHI) on blood glucose levels over time when administered subcutaneously (s.c.) to the fasted normal minipig. The figure shows that 200-2-B delivered at 5.0 nmol/kg causes less of a change in blood glucose levels that caused by RHI delivered at 0.9 nmol/kg. The figure also shows that the change in glucose levels observed for 200-2-B is less likely to result in severe hypoglycemia.

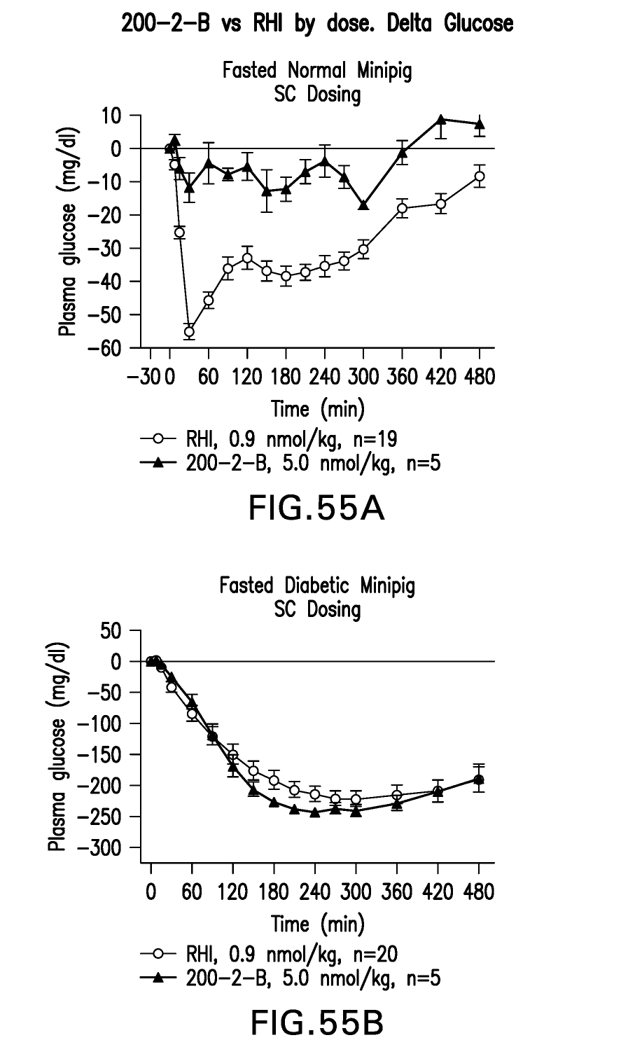

FIG. 55A shows the data shown in FIG. 54B replotted as change in blood glucose from baseline and FIG. 55B shows the data shown in and FIG. 54A replotted as change in blood glucose from baseline. These Figures show that 200-2-B is also affects blood glucose levels in a glucose-responsive manner and FIG. 55B shows that 200-2-B is controlling blood glucose levels in the fasted diabetic minipig.

### Example 16

This example shows expression of two insulin analogue precursors in the yeast Kluyveromyces lactis. The first insulin analogue precursor is a single chain precursor having the sequence EEAEAEAEPKFVNQHLCGSHLVEALYLVCGERGFFYTN*KTAAKGIVEQCCTSICSLYQLEN YCN (SEQ ID NO:305) wherein the Pro residue at B28 is substituted with Asn to generate a consensus N-glycan motif, wherein the Asn residue N* at position B28 is covalently linked in a β1 linkage to a mannosylated N-glycan. The second insulin analogue precursor is a single chain precursor having the sequence EEGHHHHHHHHHHHEPKFVNQHLCGSHLVEALYLVCGERGFFYTNKAAKGIVEQCCTSICSL YQLENYCN (SEQ ID NO:304) wherein the Pro residue at B28 is substituted with Asn but is lacking an N-glycan due to the removal of the B30 Thr residue.

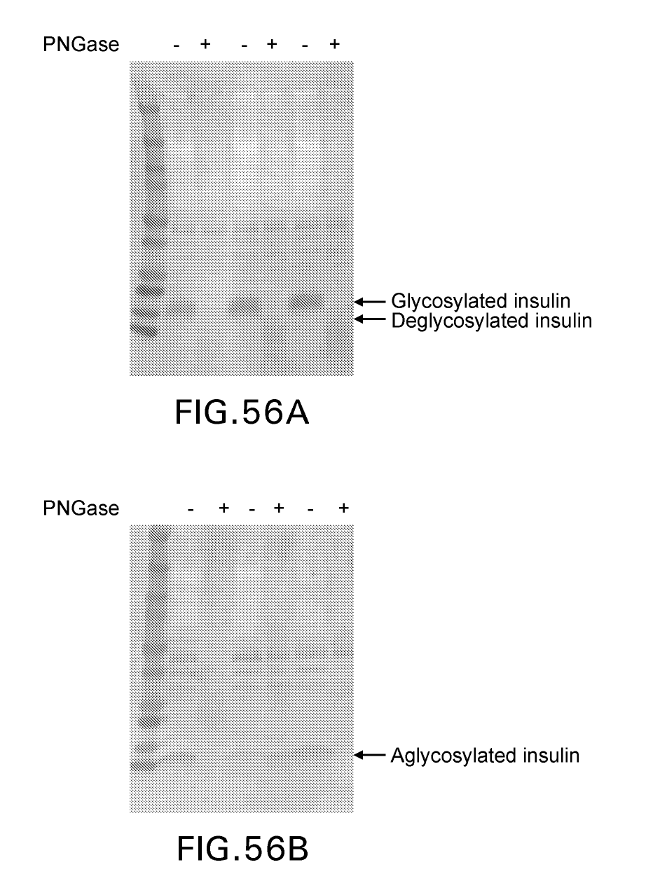

FIG. 56A shows an image of a Western blot that detects secreted insulin analogue precursor from K. lactis induced for recombinant protein expression. In this strain, the DNA, which encodes secreted insulin analogue precursor with an N-glycan at position B28 (SEQ ID NO:154), is cloned behind the KlLAC4 promoter and the resulting plasmid is transformed by electroporation into the OCH1-deficient strain K34 (See U.S. Pat. No. 7,449,308). Three random transformants were induced for insulin analogue precursor expression in media containing BMGGalY (3%) and cell-free supernatant was obtained by centrifugation. An aliquot of the cell-free supernatant was then incubated with PNGase to remove N-glycans per standard reaction conditions and applied to SDS-PAGE analysis. Proteins were transferred to a membrane and probed with an anti-insulin antibody per standard Western techniques. The results of such treatment is shown in FIG. 56A wherein the Western blot of all three supernatants of random expression clones in the absence of PNGase (denoted with “−”) reveal a cross-reactive band with higher molecular weight than those same supernatants treated with PNGase (adjacent lane denoted with “+). The data indicates the insulin analogue precursor band of SEQ ID NO:154, expressed in K. lactis, contains an N-linked glycans that is capable of deglycosylation with the enzyme PNGase.

To further verify the shift in molecular weight is due to N-glycosylation of the insulin analogue precursor and not due to the substitution at B28 with Asn, a second insulin analogue precursor gene was cloned into a K. lactis expression vector and the resulting strain was induced for protein expression. FIG. 56B shows an image of a Western blot that detects secreted insulin analogue precursor from K. lactis induced for recombinant protein expression. In this strain, the DNA, which encodes secreted insulin analogue precursor with the B:P28N substitution but lacking Thr at B30 and therefore lacks an N-glycan (SEQ ID NO:304), is cloned behind the KlLAC4 promoter and the resulting plasmid is transformed by electroporation into the OCH1-deficient strain K34. Three random transformants were induced for insulin analogue precursor expression in media containing BMGGalY (3%) and cell-free supernatant was obtained by centrifugation. An aliquot of the cell-free supernatant was then incubated with PNGase to remove N-glycans per standard reaction conditions and applied to SDS-PAGE analysis. Proteins were transferred to a membrane and probed with an anti-insulin antibody per standard Western techniques. The results of such treatment is shown in FIG. 56B wherein the Western blot of all three supernatants of random expression clones in the absence of PNGase (denoted with “−”) reveal a cross-reactive band with the same molecular weight than those same supernatants treated with PNGase (adjacent lane denoted with “+). The data indicates the insulin analogue precursor band of SEQ ID NO:304, expressed in K. lactis, does not contain an N-linked glycan since the N-glycan tripeptide motif of Asn-X-Thr/Ser, wherein X≠Pro, was eliminated by the lack of Thr residue at B30 and the molecular weight was not shifted by treatment with the enzyme PNGase.

### Example 17

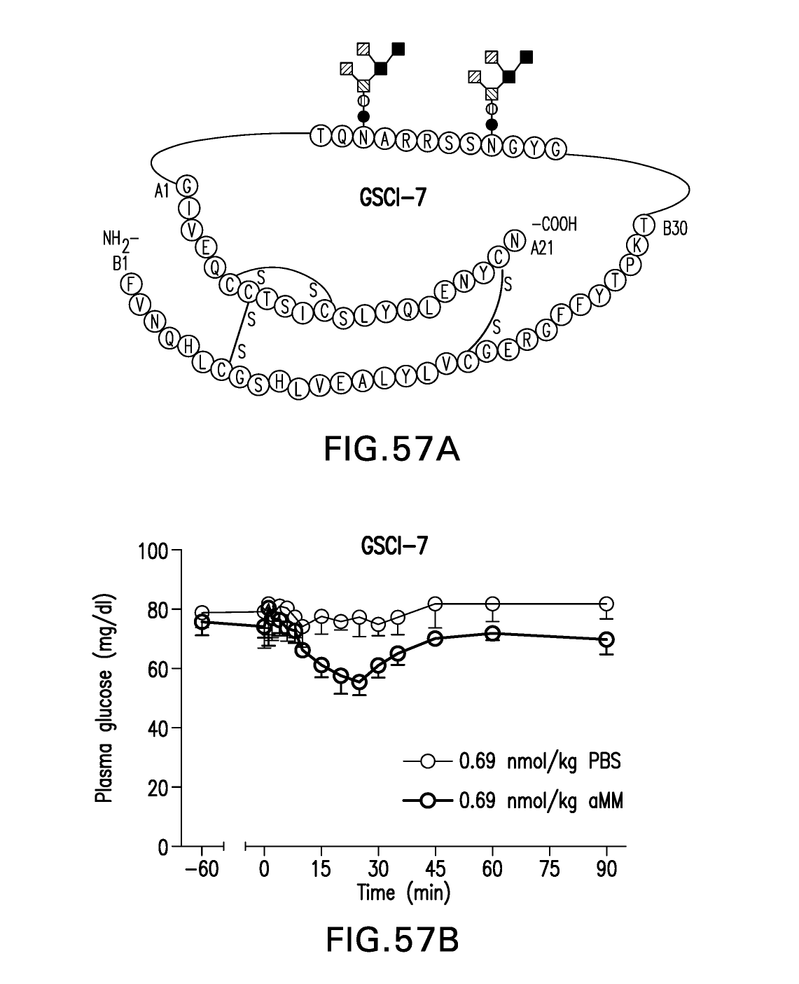

This example shows a single chain N-glycosylated insulin analogue that exhibits glucose-responsive properties. The insulin analogue is denoted GSCI-7 and is a single chain insulin analogue comprising a native insulin B-chain and a A-chain, connected by a twelve amino acid C-peptide containing two N-glycans, having the amino acid sequence FVNQHLCGSHLVEALYLVCGERGFFYTPKTGYGN*SSRRAN*QTGIVEQCCTSICSLYQLEN YCN (SEQ ID NO:303) wherein the Asn residues N* at positions 34 and 40 (C4 & C10) are each covalently linked in a β1 linkage to a Man₅GlcNAc₂ N-glycan, as illustrated in FIG. 57A.

The insulin analogue GSCI-7 was generated by transforming a plasmid containing a DNA expression cassette that encodes the GSCI-7 protein sequence into the host strain YGLY24962, which has the same genotype and genetic modifications as YGLY24964 previously described in FIG. 49B. The resulting strain was fermented and purified to obtain the single chain insulin analogue GSCI-7 containing two N-glycans. The analogue GSCI-7 was not processed by LysC, trypsin, or another endoproteinase to retain single chain properties prior to being assayed for activity.

To assess the activity of GSCI-7, three in vitro assays were performed. Binding to the human insulin receptor isoform B (IR-b) was determined in a competition of the analog with radiolabeled human insulin to Chinese hamster ovary (CHO) cells over-expressing IR-b and presented as an IC50 value. Functional activation of IR-b was determined by assessing the phosphorylation of IR-b in Chinese hamster ovary (CHO) cells over-expressing IR-b and presented as an EC50 value Binding to the human mannose receptor C type 1 (MRC1) was determined in a competition of the analog with europium-labeled mannose-BSA to the ectodomain of MRC1 in an ELISA assay and presented as an IC50 value. The in vitro properties of IR-b binding, IR-b phosphorylation, and MRC1 binding of the analogues compared to the binding of recombinant human insulin (RHI) are shown in Table 8.

TABLE 8

| | Human IRb | Human IRb | Human MRC1 Bound |
|---|---|---|---|
| Analogue | Bound (nM) | Phosphorylation (nM) | (nM) |
| GSCI-7 | 28.4 | 39.4 | 2.93 |
| RHI | 0.2 | 0.3 | >10000 |

To study the glucose responsiveness of GSCI-7, two non-diabetic Yucatan minipigs were fasted overnight before dosed by intravenous injection with 0.69 nmol//kg GSCI-7. At the same time, animals received intravenous administration of sterile phosphate-buffered saline (PBS) (2.67 ml/kg/hr) or sterile α-methylmannose solution (αMM) (21.2% w/v in phosphate-buffered saline at a rate of 2.67 ml/kg/hr). At high concentrations, α-methylmannose (αMM) is known to competitively inhibit interactions between c-type lectins and glycoproteins, especially those terminating in mannose, GlcNAc, or fucose residues. Blood glucose was measured using a handheld glucometer at times −60, 0, 1, 2, 4, 6, 8, 10, 15, 20, 25, 30, 35, 45, 60, and 90 minutes post injection.

As shown in FIG. 57B, GSCI-7 containing N-glycans with terminal mannose dosed at 0.69 nmol/kg did not appreciably lower blood glucose during the 90 minute study period when co-injected with PBS. However, the co-injection of α-methylmannose with the same dose of GSCI-7 lowered glucose with better or greater potency. Glucose is known to inhibit interactions between mannose-binding c-type lectins and glycoproteins, albeit with less potency than α-methylmannose. These data show that the single chain analogue GSCI-7 is able to lower blood glucose levels in a glucose responsive fashion, likely mediated by mannose binding lectins such as mannose receptor.

Table of Sequences

| SEQ | | |
|---|---|---|
| ID | | |
| NO: | Description | Sequence |
| 1 | MAM508 | CATCATTATTAGCTTACTTTCATAATTGC |
| 2 | MAM509 | CATGCGTACACGCGTTTGTACAG |
| 3 | MAM564 | GCAAAAGGCCGGCCTTATTAACCGCAGTAGTTCTCCAATTGGTAC |
| 4 | MAM864 | AAAAGAGTCCTCTTGAAGAAGGTCACCACCATCACCATCATCACCA |
| | | TCATCACGAACCAAAGTTTGTTAATCAACACTTGTGTGG |
| 5 | DNA encoding pre- | ATGAAGTTGAAGACTGTTAGATCCGCTGTTTTGTCTTCTTTGTTTGCT |
| | proinsulin analogue: | TCTCAAGTTTTGGGTCAACCAATTGATGATACTGAATCTCAAACTAC |
| | Yps1ss + TA57 | TTCTGTTAACTTGATGGCTGATGATACTGAATCTGCTTTTGCTACTC |
| | propeptide + N- | AAACTAACTCTGGTGGTTTGGATGTTGTTGGTTTGATTTCTATGGCT |
| | terminal spacer + B | AAGAGAGAAGAAGGTGAACCAAAGTTTGTTAACCAACATTTGTGTG |
| | chain P28N + C- | GTTCTCATTTGGTTGAAGCTTTGTACTTGGTTTGTGGTGAAAGAGGT |
| | peptide “AAK” + | TTTTTTTACACTAACAAGACTGCTGCTAAGGGTATTGTTGAACAATG |
| | insulin A chain | TTGTACTTCTATTTGTTCTTTGTACCAATTGGAAAACTACTGTAACTA |
| | | A |
| 6 | Pre-proinsulin | MKLKTVRSAVLSSLFASQVLGQPIDDTESQTTSVNLMADDTESAFATQ |
| | analogue: | TNSGGLDVVGLISMAKREEGEPKFVNQHLCGSHLVEALYLVCGERGFF |
| | Yps1ss + TA57 | YTNKTAAKGIVEQCCTSICSLYQLENYCN |
| | propeptide + N- | |
| | terminal spacer + B | |
| | chain P28N + C- | |
| | peptide “AAK” + | |
| | insulin A chain | |
| 7 | DNA encoding pre- | ATGAAGTTGAAGACTGTTAGATCCGCTGTTTTGTCTTCTTTGTTTGCT |
| | proinsulin analogue: | TCTCAAGTTTTGGGTCAACCAATTGATGATACTGAATCTCAAACTAC |
| | S.c. alpha mating | TTCTGTTAACTTGATGGCTGATGATACTGAATCTGCTTTTGCTACTC |
| | factor signal | AAACTAACTCTGGTGGTTTGGATGTTGTTGGTTTGATTTCTATGGCT |
| | sequence and pro- | AAGAGAGAAGAAGGTGAACCAAAGTTTGTTAACCAACATTTGTGTG |
| | peptide + N-terminal | GTTCTCATTTGGTTGAAGCTTTGTACTTGGTTTGTGGTGAAAGAGGT |
| | spacer + B chain | TTTTTTTACACTAACAAGACTGCTCACCACCATCACCATCATCACCA |
| | P28N + C-peptide | TCATCACGCTAAGGGTATTGTTGAACAATGTTGTACTTCTATTTGTT |
| | “A(10xHIS)AK” + | CTTTGTACCAATTGGAAAACTACTGTAACTAA |
| | insulin A chain | |
| 8 | Pre-proinsulin | MKLKTVRSAVLSSLFASQVLGQPIDDTESQTTSVNLMADDTESAFATQ |
| | analogue: | TNSGGLDVVGLISMAKREEGEPKFVNQHLCGSHLVEALYLVCGERGFF |
| | Yps1ss + TA57 | YTNKTAHHHHHHHHHHAKGIVEQCCTSICSLYQLENYCN |
| | propeptide + N- | |
| | terminal spacer + B | |
| | chain P28N + C- | |
| | peptide | |
| | “A(10xHIS)AK” + | |
| | insulin A chain | |
| 9 | DNA encoding pre- | ATGAGATTTCCTTCAATTTTTACTGCAGTTTTATTCGCAGCATCCTCC |
| | proinsulin analogue: | GCATTAGCTGCTCCAGTCAACACTACAACAGAAGATGAAACGGCAC |
| | S.c. alpha mating | AAATTCCGGCTGAAGCTGTCATCGGTTACTCAGATTTAGAAGGGGA |
| | factor signal | TTTCGATGTTGCTGTTTTGCCATTTTCCAACAGCACAAATAACGGGT |
| | sequence and pro- | TATTGTTTATAAATACTACTATTGCCAGCATTGCTGCTAAAGAAGAA |
| | peptide + B chain | GGGGTATCTCTCGAGAAAAGGTTTGTTAATCAACACTTGTGTGGTTC |
| | P28N + C-peptide | CCACTTGGTTGAGGCTTTGTACTTGGTTTGTGGTGAGAGAGGTTTCT |
| | “RR” + A chain | TCTACACTAACAAGACTAGAAGAGGTATCGTTGAGCAGTGTTGTAC |
| | | TTCCATCTGTTCCTTGTACCAGTTGGAGAACTACTGTAACTAA |
| 10 | Pre-proinsulin | MRFPSIFTAVLFAASSALAAPVNTTTEDETAQIPAEAVIGYLDLEGDFD |
| | analogue: S.c. alpha | VAVLPFSNSTNNGLLFINTTIASIAAKEEGVSLEKRFVNQHLCGSHLVE |
| | mating factor signal | ALYLVCGERGFFYTNKTRRGIVEQCCTSICSLYQLENYCN |
| | sequence and pro- | |
| | peptide + B chain | |
| | P28N + C-peptide | |
| | “RR” + A chain | |
| 11 | DNA encoding pre- | ATGAGATTTCCTTCAATTTTTACTGCAGTTTTATTCGCAGCATCCTCC |
| | proinsulin analogue: | GCATTAGCTGCTCCAGTCAACACTACAACAGAAGATGAAACGGCAC |
| | S.c. alpha mating | AAATTCCGGCTGAAGCTGTCATCGGTTACTCAGATTTAGAAGGGGA |
| | factor signal | TTTCGATGTTGCTGTTTTGCCATTTTCCAACAGCACAAATAACGGGT |
| | sequence and pro- | TATTGTTTATAAATACTACTATTGCCAGCATTGCTGCTAAAGAAGAA |
| | peptide + B chain | GGGGTATCTCTCGAGAAAAGGTTTGTTAATCAACACTTGTGTGGTTC |
| | P28N + C-peptide | CCACTTGGTTGAGGCTTTGTACTTGGTTTGTGGTGAGAGAGGTTTCT |
| | “RR” + glargine A | TCTACACTAACAAGACTAGAAGAGGTATCGTTGAGCAGTGTTGTAC |
| | chain N21G | TTCCATCTGTTCCTTGTACCAATTGGAGAACTACTGCGGTTAA |
| 12 | Pre-proinsulin | MRFPSIFTAVLFAASSALAAPVNTTTEDETAQIPAEAVIGYLDLEGDFD |
| | analogue: S.c. alpha | VAVLPFSNSTNNGLLFINTTIASIAAKEEGVSLEKRFVNQHLCGSHLVE |
| | mating factor signal | ALYLVCGERGFFYTNKTRRGIVEQCCTSICSLYQLENYCG |
| | sequence and pro- | |
| | peptide + B chain | |
| | P28N + C-peptide | |
| | “RR” + glargine A | |
| | chain N21G | |
| 13 | DNA encoding pre- | ATGAGATTTCCTTCAATTTTTACTGCAGTTTTATTCGCAGCATCCTCC |
| | proinsulin analogue: | GCATTAGCTGCTCCAGTCAACACTACAACAGAAGATGAAACGGCAC |
| | S.c. alpha mating | AAATTCCGGCTGAAGCTGTCATCGGTTACTCAGATTTAGAAGGGGA |
| | factor signal | TTTCGATGTTGCTGTTTTGCCATTTTCCAACAGCACAAATAACGGGT |
| | sequence and pro- | TATTGTTTATAAATACTACTATTGCCAGCATTGCTGCTAAAGAAGAA |
| | peptide + N-terminal | GGGGTATCTCTCGAGAAAAGGGAAGAAGGTCACCACCATCACCATC |
| | HIS spacer + B chain | ATCACCATCATCACGAACCAAAGTTTGTTAATCAACACTTGTGTGGT |
| | P28N + C-peptide | TCCCACTTGGTTGAGGCTTTGTACTTGGTTTGTGGTGAGAGAGGTTT |
| | “RR” + glargine A | CTTCTACACTAACAAGACTAGAAGAGGTATCGTTGAGCAGTGTTGT |
| | chain N21G | ACTTCCATCTGTTCCTTGTACCAATTGGAGAACTACTGCGGTTAA |
| 14 | Pre-proinsulin | MRFPSIFTAVLFAASSALAAPVNTTTEDETAQIPAEAVIGYLDLEGDFD |
| | analogue: S.c. alpha | VAVLPFSNSTNNGLLFINTTIASIAAKEEGVSLEKREEGHHHHHHHHHH |
| | mating factor signal | EPKFVNQHLCGSHLVEALYLVCGERGFFYTNKTRRGIVEQCCTSICSLY |
| | sequence and pro- | QLENYCG |
| | peptide + N-terminal | |
| | HIS spacer + B chain | |
| | P28N + C-peptide | |
| | “RR” + glargine A | |
| | chain N21G | |
| 15 | DNA encoding pre- | ATGAGATTCCCATCCATCTTCACTGCTGTTTTGTTCGCTGCTTCCTCT |
| | proinsulin analogue: | GCTTTGGCTGCTCCAGTTAACACTACTACTGAGGACGAGACTGCTCA |
| | S.c. alpha mating | GATTCCAGCTGAAGCTGTTATCGGTTACTTGGACTTGGAGGGTGACT |
| | factor signal | TCGACGTTGCTGTTTTGCCATTCTCCAACTCCACTAACAACGGTTTG |
| | sequence and pro- | TTGTTCATCAACACTACTATCGCTTCCATTGCTGCTAAAGAAGAGGG |
| | peptide + N-terminal | AGTTTCCTTGGAGAAGAGAGAGGAACAGAAGTTGATCTCCGAAGA |
| | MYC spacer + B | GGACTTGAACGAGAAGTTCGTTAACCAGCACTTGTGTGGTTCCCAC |
| | chain P28N + C- | TTGGTTGAGGCTTTGTACTTGGTTTGTGGTGAGAGAGGTTTCTTCTA |
| | peptide | CACTAACAAGACTACTGCTCATCACCATCACCATCATCACCACCATC |
| | “TA(10xHIS)AK” + | ACGCTAAGGGTATCGTTGAGCAGTGTTGTACTTCCATCTGTTCCTTG |
| | A chain | TACCAGTTGGAGAACTACTGTAACTAA |
| 16 | Pre-proinsulin | MRFPSIFTAVLFAASSALAAPVNTTTEDETAQIPAEAVIGYLDLEGDFD |
| | analogue: S.c. alpha | VAVLPFSNSTNNGLLFINTTIASIAAKEEGVSLEKREEQKLISEEDLNEKF |
| | mating factor signal | VNQHLCGSHLVEALYLVCGERGFFYTNKTTAHHHHHHHHHHAKGIVE |
| | sequence and pro- | QCCTSICSLYQLENYCN |
| | peptide + N-terminal | |
| | MYC spacer + B | |
| | chain P28N + C- | |
| | peptide | |
| | “TA(10xHIS)AK” + | |
| | A chain | |
| 17 | DNA encoding pre- | ATGAGATTTCCATCTATTTTTACTGCTGTTTTGTTTGCTGCTTCTTCT |
| | proinsulin analogue: | GCTTTGGCTGCTCCAGTTAACACTACTACTGAAGATGAAACTGCTCA |
| | S.c. alpha mating | AATTCCAGCTGAAGCTGTTATTGGTTACTTGGATTTGGAAGGTGATT |
| | factor signal | TTGATGTTGCTGTTTTGCCATTTTCTAACTCTACTAACAACGGTTTGT |
| | sequence and pro- | TGTTTATTAACACTACTATTGCTTCTATTGCTGCTAAGGAAGAAGGT |
| | peptide + N-terminal | GTTTCTTTGGAAAAGAGAGAAGAACAAAAGTTGATTTCTGAAGAAG |
| | MYC spacer + B | ATTTGAACGAAAAGTTTGTTAACCAACATTTGTGTGGTTCTCATTTG |
| | chain P28N + C- | GTTGAAGCTTTGTACTTGGTTTGTGGTGAAAGAGGTTTTTTTTACAC |
| | peptide | TAACAAGACTACTGCTCATCATCATCATCATCATCATCATCATCATG |
| | “TA(10xHIS)AK” + | CTAAGGGTATTGTTGAACAATGTTGTACTTCTATTTGTTCTTTGTACC |
| | A chain; alternate | AATTGGAAAACTACTGTAACTAA |
| | DNA codon | |
| | optimization | |
| 18 | Pre-proinsulin | MRFPSIFTAVLFAASSALAAPVNTTTEDETAQIPAEAVIGYLDLEGDFD |
| | analogue: S.c. alpha | VAVLPFSNSTNNGLLFINTTIASIAAKEEGVSLEKREEQKLISEEDLNEKF |
| | mating factor signal | VNQHLCGSHLVALYLVCGERGFFYTNKTTAHHHHHHHHHHAKGIVE |
| | sequence and pro- | QCCTSICSLYQLENYCN |
| | peptide + N-terminal | |
| | MYC spacer + B | |
| | chain P28N + C- | |
| | peptide | |
| | “TA(10xHIS)AK” + | |
| | A chain; alternate | |
| | DNA codon | |
| | optimization | |
| 19 | Sc alpha mating | MRFPSIFTAVLFAASSALAAPVNTTTEDETAQIPAEAVIGYLDLEGDFD |
| | factor signal | VAVLPFSNSTNNGLLFINTTIASIAAKEEGVSLEKR |
| | sequence and pro- | |
| | peptide | |
| 20 | Yps1ss | MKLKTVRSAVLSSLFASQVLG |
| 21 | TA57 pro | QPIDDTESQTTSVNLMADDTESAFATQTNSGGLDVVGLISMAKR |
| 22 | N-terminal spacer | EEGEPK |
| 23 | N-terminal HIS | EEGHHHHHHHHHHEPK |
| | spacer | |
| 24 | N-terminal MYC | EEQKLISEEDLNEK |
| | spacer | |
| 25 | Human insulin B | FVNQHLCGSHLVEALYLVCGERGFFYTPKT |
| | chain | |
| 26 | Insulin B chain with | FVNQHLCGSHLVEALYLVCGERGFFYTNKT |
| | P28N | |
| 27 | Insulin glargine B | FVNQHLCGSHLVEALYLVCGERGFFYTPKTRR |
| | chain | |
| 28 | insulin glargine | FVNQHLCGSHLVEALYLVCGERGFFYTNKTRRGIVEQCCTSICSLYQLE |
| | proinsulin (B chain | NYCG |
| | P28N) | |
| 29 | Insulin glargine | FVKQHLCGSHLVEALYLVCGERGFFYTNKTRRGIVEQCCTSICSLYQLE |
| | proinsulin with | NYCG |
| | glulisine mutation (B | |
| | chain N3K) and (B | |
| | chain P28N) | |
| 30 | Human insulin C | RREAEDLQVGQVELGGGPGAGSLQPLALEGSLQKR |
| | chain | |
| 31 | C peptide “AAK” | AAK |
| 32 | C peptide “HIS” | AHHHHHHHHHHAK |
| 33 | Human insulin A | GIVEQCCTSICSLYQLENYCN |
| | chain | |
| 34 | Insulin glargine A | GIVEQCCTSICSLYQLENYCG |
| | chain N21G | |
| 35 | Human pre- | MALWMRLLPLLALLALWGPDPAAAFVNQHLCGSHLVEALYLVCGER |
| | proinsulin | GFFYTPKTRREAEDLQVGQVELGGGPGAGSLQPLALEGSLQKRGIVEQ |
| | | CCTSICSLYQLENYCN |
| 36 | Insulin proinsulin | EEGEPKFVNQHLCGSHLVEALYLVCGERGFFYTNKTAAKGIVEQCCTS |
| | with N-terminal | ICSLYQLENYCN |
| | spacer and C-peptide | |
| | “AAK” and B chain | |
| | P28N glycosylation | |
| | site | |
| 37 | Insulin proinsulin | FVNQHLCGSHLVEALYLVCGERGFFYTNKTAAKGIVEQCCTSICSLYQ |
| | with C-peptide | LENYCN |
| | “AAK” and B chain | |
| | P28N glycosylation | |
| | site | |
| 38 | Iinsulin proinsulin | FVNQHLCGSHLVEALYLVCGERGFFYTNKTRRGIVEQCCTSICSLYQLE |
| | with C-chain “RR” | NYCN |
| | and B chain P28N | |
| | glycosylation site | |
| 39 | Insulin proinsulin | EEGEPKFVNQHLCGSHLVEALYLVCGERGFFYTNKTAHHHHHHHHHH |
| | with N-terminal | AKGIVEQCCTSICSLYQLENYCN |
| | spacer and C-peptide | |
| | “A(10xHIS)AK” and | |
| | B chain P28N | |
| | glycosylation site | |
| 40 | Insulin proinsulin | EEQKLISEEDLNEKFVNQHLCGSHLVEALYLVCGERGFFYTNKTTAHH |
| | with N-terminal | HHHHHHHHAKGIVEQCCTSICSLYQLENYCN |
| | spacer (myc epitope) | |
| | and C-peptide | |
| | “A(10xHIS)AK” and | |
| | B chain P28N | |
| | glycosylation site | |
| 41 | Insulin glargine | EEGHHHHHHHHHHEPKFVNQHLCGSHLVEALYLVCGERGFFYTNKTR |
| | proinsulin with N- | RGIVEQCCTSICSLYQLENYCG |
| | terminal HIS spacer | |
| | and B chain P28N | |
| | glycosylation site | |
| 42 | B chain H5S: | FVNQSLCGSHLVEALYLVCGERGFFYTPKT |
| 43 | B chain H5T: | FVNQTLCGSHLVEALYLVCGERGFFYTPKT |
| 44 | B chain F25N: | FVNQHLCGSHLVEALYLVCGERGFNYTPKT |
| 45 | A chain I10N: | GIVEQCCTSNCSLYQLENYCN |
| 46 | S. cerevisiae | AGGCCTCGCAACAACCTATAATTGAGTTAAGTGCCTTTCCAAGCTA |
| | invertase gene | AAAAGTTTGAGGTTATAGGGGCTTAGCATCCACACGTCACAATCTC |
| | (ScSUC2) ORF | GGGTATCGAGTATAGTATGTAGAATTACGGCAGGAGGTTTCCCAAT |
| | underlined | GAACAAAGGACAGGGGCACGGTGAGCTGTCGAAGGTATCCATTTTA |
| | | TCATGTTTCGTTTGTACAAGCACGACATACTAAGACATTTACCGTAT |
| | | GGGAGTTGTTGTCCTAGCGTAGTTCTCGCTCCCCCAGCAAAGCTCAA |
| | | AAAAGTACGTCATTTAGAATAGTTTGTGAGCAAATTACCAGTCGGT |
| | | ATGCTACGTTAGAAAGGCCCACAGTATTCTTCTACCAAAGGCGTGC |
| | | CTTTGTTGAACTCGATCCATTATGAGGGCTTCCATTATTCCCCGCAT |
| | | TTTTATTACTCTGAACAGGAATAAAAAGAAAAAACCCAGTTTAGGA |
| | | AATTATCCGGGGGCGAAGAAATACGCGTAGCGTTAATCGACCCCAC |
| | | GTCCAGGGTTTTTCCATGGAGGTTTCTGGAAAAACTGACGAGGAAT |
| | | GTGATTATAAATCCCTTTATGTGATGTCTAAGACTTTTAAGGTACGC |
| | | CCGATGTTTGCCTATTACCATCATAGAGACGTTTCTTTTCGAGGAAT |
| | | GCTTAAACGACTTTGTTTGACAAAAATGTTGCCTAAGGGCTCTATAG |
| | | TAAACCATTTGGAAGAAAGATTTGACGACTTTTTTTTTTTGGATTTC |
| | | GATCCTATAATCCTTCCTCCTGAAAAGAAACATATAAATAGATATG |
| | | TATTATTCTTCAAAACATTCTCTTGTTCTTGTGCTTTTTTTTTACCATA |
| | | TATCTTACTTTTTTTTTTCTCTCAGAGAAACAAGCAAAACAAAAAGC |
| | | TTTTCTTTTCACTAACGTATATGATGCTTTTGCAAGCTTTCCTTTTCC |
| | | TTTTGGCTGGTTTTGCAGCCAAAATATCTGCATCAATGACAAACGA |
| | | AACTAGCGATAGACCTTTGGTCCACTTCACACCCAACAAGGGCTGG |
| | | ATGAATGACCCAAATGGGTTGTGGTACGATGAAAAAGATGCCAAAT |
| | | GGCATCTGTACTTTCAATACAACCCAAATGACACCGTATGGGGTAC |
| | | GCCATTGTTTTGGGGCCATGCTACTTCCGATGATTTGACTAATTGGG |
| | | AAGATCAACCCATTGCTATCGCTCCCAAGCGTAACGATTCAGGTGC |
| | | TTTCTCTGGCTCCATGGTGGTTGATTACAACAACACGAGTGGGTTTT |
| | | TCAATGATACTATTGATCCAAGACAAAGATGCGTTGCGATTTGGAC |
| | | TTATAACACTCCTGAAAGTGAAGAGCAATACATTAGCTATTCTCTTG |
| | | ATGGTGGTTACACTTTTACTGAATACCAAAAGAACCCTGTTTTAGCT |
| | | GCCAACTCCACTCAATTCAGAGATCCAAAGGTGTTCTGGTATGAAC |
| | | CTTCTCAAAAATGGATTATGACGGCTGCCAAATCACAAGACTACAA |
| | | AATTGAAATTTACTCCTCTGATGACTTGAAGTCCTGGAAGCTAGAAT |
| | | CTGCATTTGCCAATGAAGGTTTCTTAGGCTACCAATACGAATGTCCA |
| | | GGTTTGATTGAAGTCCCAACTGAGCAAGATCCTTCCAAATCTTATTG |
| | | GGTCATGTTTATTTCTATCAACCCAGGTGCACCTGCTGGCGGTTCCT |
| | | TCAACCAATATTTTGTTGGATCCTTCAATGGTACTCATTTTGAAGCG |
| | | TTTGACAATCAATCTAGAGTGGTAGATTTTGGTAAGGACTACTATGC |
| | | CTTGCAAACTTTCTTCAACACTGACCCAACCTACGGTTCAGCATTAG |
| | | GTATTGCCTGGGCTTCAAACTGGGAGTACAGTGCCTTTGTCCCAACT |
| | | AACCCATGGAGATCATCCATGTCTTTGGTCCGCAAGTTTTCTTTGAA |
| | | CACTGAATATCAAGCTAATCCAGAGACTGAATTGATCAATTTGAAA |
| | | GCCGAACCAATATTGAACATTAGTAATGCTGGTCCCTGGTCTCGTTT |
| | | TGCTACTAACACAACTCTAACTAAGGCCAATTCTTACAATGTCGATT |
| | | TGAGCAACTCGACTGGTACCCTAGAGTTTGAGTTGGTTTACGCTGTT |
| | | AACACCACACAAACCATATCCAAATCCGTCTTTGCCGACTTATCACT |
| | | TTGGTTCAAGGGTTTAGAAGATCCTGAAGAATATTTGAGAATGGGT |
| | | TTTGAAGTCAGTGCTTCTTCCTTCTTTTTGGACCGTGGTAACTCTAAG |
| | | GTCAAGTTTGTCAAGGAGAACCCATATTTCACAAACAGAATGTCTG |
| | | TCAACAACCAACCATTCAAGTCTGAGAACGACCTAAGTTACTATAA |
| | | AGTGTACGGCCTACTGGATCAAAACATCTTGGAATTGTACTTCAAC |
| | | GATGGAGATGTGGTTTCTACAAATACCTACTTCATGACCACCGGTA |
| | | ACGCTCTAGGATCTGTGAACATGACCACTGGTGTCGATAATTTGTTC |
| | | TACATTGACAAGTTCCAAGTAAGGGAAGTAAAATAGAGGTTATAA |
| | | AACTTATTGTCTTTTTTATTTTTTTCAAAAGCCATTCTAAAGGGCTTT |
| | | AGCTAACGAGTGACGAATGTAAAACTTTATGATTTCAAAGAATACC |
| | | TCCAAACCATTGAAAATGTATTTTTATTTTTATTTTCTCCCGACCCCA |
| | | GTTACCTGGAATTTGTTCTTTATGTACTTTATATAAGTATAATTCTCT |
| | | TAAAAATTTTTACTACTTTGCAATAGACATCATTTTTTCACGTAATA |
| | | AACCCACAATCGTAATGTAGTTGCCTTACACTACTAGGATGGACCTT |
| | | TTTGCCTTTATCTGTTTTGTTACTGACACAATGAAACCGGGTAAAGT |
| | | ATTAGTTATGTGAAAATTTAAAAGCATTAAGTAGAAGTATACCATA |
| | | TTGTAAAAAAAAAAAGCGTTGTCTTCTACGTAAAAGTGTTCTCAAA |
| | | AAGAAGTAGTGAGGGAAATGGATACCAAGCTATCTGTAACAGGAG |
| | | CTAAAAAATCTCAGGGAAAAGCTTCTGGTTTGGGAAACGGTCGAC |
| 47 | Sequence of the 5′- | ATCGGCCTTTGTTGATGCAAGTTTTACGTGGATCATGGACTAAGGA |
| | Region used for | GTTTTATTTGGACCAAGTTCATCGTCCTAGACATTACGGAAAGGGTT |
| | knock out of | CTGCTCCTCTTTTTGGAAACTTTTTGGAACCTCTGAGTATGACAGCT |
| | PpURA5: | TGGTGGATTGTACCCATGGTATGGCTTCCTGTGAATTTCTATTTTTTC |
| | | TACATTGGATTCACCAATCAAAACAAATTAGTCGCCATGGCTTTTTG |
| | | GCTTTTGGGTCTATTTGTTTGGACCTTCTTGGAATATGCTTTGCATAG |
| | | ATTTTTGTTCCACTTGGACTACTATCTTCCAGAGAATCAAATTGCAT |
| | | TTACCATTCATTTCTTATTGCATGGGATACACCACTATTTACCAATG |
| | | GATAAATACAGATTGGTGATGCCACCTACACTTTTCATTGTACTTTG |
| | | CTACCCAATCAAGACGCTCGTCTTTTCTGTTCTACCATATTACATGG |
| | | CTTGTTCTGGATTTGCAGGTGGATTCCTGGGCTATATCATGTATGAT |
| | | GTCACTCATTACGTTCTGCATCACTCCAAGCTGCCTCGTTATTTCCA |
| | | AGAGTTGAAGAAATATCATTTGGAACATCACTACAAGAATTACGAG |
| | | TTAGGCTTTGGTGTCACTTCCAAATTCTGGGACAAAGTCTTTGGGAC |
| | | TTATCTGGGTCCAGACGATGTGTATCAAAAGACAAATTAGAGTATT |
| | | TATAAAGTTATGTAAGCAAATAGGGGCTAATAGGGAAAGAAAAAT |
| | | TTTGGTTCTTTATCAGAGCTGGCTCGCGCGCAGTGTTTTTCGTGCTCC |
| | | TTTGTAATAGTCATTTTTGACTACTGTTCAGATTGAAATCACATTGA |
| | | AGATGTCACTCGAGGGGTACCAAAAAAGGTTTTTGGATGCTGCAGT |
| | | GGCTTCGC |
| 48 | Sequence of the 3′- | GGTCTTTTCAACAAAGCTCCATTAGTGAGTCAGCTGGCTGAATCTTA |
| | Region used for | TGCACAGGCCATCATTAACAGCAACCTGGAGATAGACGTTGTATTT |
| | knock out of | GGACCAGCTTATAAAGGTATTCCTTTGGCTGCTATTACCGTGTTGAA |
| | PpURA5: | GTTGTACGAGCTCGGCGGCAAAAAATACGAAAATGTCGGATATGCG |
| | | TTCAATAGAAAAGAAAAGAAAGACCACGGAGAAGGTGGAAGCATC |
| | | GTTGGAGAAAGTCTAAAGAATAAAAGAGTACTGATTATCGATGATG |
| | | TGATGACTGCAGGTACTGCTATCAACGAAGCATTTGCTATAATTGG |
| | | AGCTGAAGGTGGGAGAGTTGAAGGTAGTATTATTGCCCTAGATAGA |
| | | ATGGAGACTACAGGAGATGACTCAAATACCAGTGCTACCCAGGCTG |
| | | TTAGTCAGAGATATGGTACCCCTGTCTTGAGTATAGTGACATTGGAC |
| | | CATATTGTGGCCCATTTGGGCGAAACTTTCACAGCAGACGAGAAAT |
| | | CTCAAATGGAAACGTATAGAAAAAAGTATTTGCCCAAATAAGTATG |
| | | AATCTGCTTCGAATGAATGAATTAATCCAATTATCTTCTCACCATTA |
| | | TTTTCTTCTGTTTCGGAGCTTTGGGCACGGCGGCGGGTGGTGCGGGC |
| | | TCAGGTTCCCTTTCATAAACAGATTTAGTACTTGGATGCTTAATAGT |
| | | GAATGGCGAATGCAAAGGAACAATTTCGTTCATCTTTAACCCTTTCA |
| | | CTCGGGGTACACGTTCTGGAATGTACCCGCCCTGTTGCAACTCAGGT |
| | | GGACCGGGCAATTCTTGAACTTTCTGTAACGTTGTTGGATGTTCAAC |
| | | CAGAAATTGTCCTACCAACTGTATTAGTTTCCTTTTGGTCTTATATTG |
| | | TTCATCGAGATACTTCCCACTCTCCTTGATAGCCACTCTCACTCTTCC |
| | | TGGATTACCAAAATCTTGAGGATGAGTCTTTTCAGGCTCCAGGATG |
| | | CAAGGTATATCCAAGTACCTGCAAGCATCTAATATTGTCTTTGCCAG |
| | | GGGGTTCTCCACACCATACTCCTTTTGGCGCATGC |
| 49 | Sequence of the | TCTAGAGGGACTTATCTGGGTCCAGACGATGTGTATCAAAAGACAA |
| | PpURA5 | ATTAGAGTATTTATAAAGTTATGTAAGCAAATAGGGGCTAATAGGG |
| | auxotrophic marker: | AAAGAAAAATTTTGGTTCTTTATCAGAGCTGGCTCGCGCGCAGTGTT |
| | | TTTCGTGCTCCTTTGTAATAGTCATTTTTGACTACTGTTCAGATTGAA |
| | | ATCACATTGAAGATGTCACTGGAGGGGTACCAAAAAAGGTTTTTGG |
| | | ATGCTGCAGTGGCTTCGCAGGCCTTGAAGTTTGGAACTTTCACCTTG |
| | | AAAAGTGGAAGACAGTCTCCATACTTCTTTAACATGGGTCTTTTCAA |
| | | CAAAGCTCCATTAGTGAGTCAGCTGGCTGAATCTTATGCTCAGGCC |
| | | ATCATTAACAGCAACCTGGAGATAGACGTTGTATTTGGACCAGCTT |
| | | ATAAAGGTATTCCTTTGGCTGCTATTACCGTGTTGAAGTTGTACGAG |
| | | CTGGGCGGCAAAAAATACGAAAATGTCGGATATGCGTTCAATAGAA |
| | | AAGAAAAGAAAGACCACGGAGAAGGTGGAAGCATCGTTGGAGAAA |
| | | GTCTAAAGAATAAAAGAGTACTGATTATCGATGATGTGATGACTGC |
| | | AGGTACTGCTATCAACGAAGCATTTGCTATAATTGGAGCTGAAGGT |
| | | GGGAGAGTTGAAGGTTGTATTATTGCCCTAGATAGAATGGAGACTA |
| | | CAGGAGATGACTCAAATACCAGTGCTACCCAGGCTGTTAGTCAGAG |
| | | ATATGGTACCCCTGTCTTGAGTATAGTGACATTGGACCATATTGTGG |
| | | CCCATTTGGGCGAAACTTTCACAGCAGACGAGAAATCTCAAATGGA |
| | | AACGTATAGAAAAAAGTATTTGCCCAAATAAGTATGAATCTGCTTC |
| | | GAATGAATGAATTAATCCAATTATCTTCTCACCATTATTTTCTTCTGT |
| | | TTCGGAGCTTTGGGCACGGCGGCGGATCC |
| 50 | Sequence of the part | CCTGCACTGGATGGTGGCGCTGGATGGTAAGCCGCTGGCAAGCGGT |
| | of the Ec lacZ gene | GAAGTGCCTCTGGATGTCGCTCCACAAGGTAAACAGTTGATTGAAC |
| | that was used to | TGCCTGAACTACCGCAGCCGGAGAGCGCCGGGCAACTCTGGCTCAC |
| | construct the | AGTACGCGTAGTGCAACCGAACGCGACCGCATGGTCAGAAGCCGG |
| | PpURA5 blaster | GCACATCAGCGCCTGGCAGCAGTGGCGTCTGGCGGAAAACCTCAGT |
| | (recyclable | GTGACGCTCCCCGCCGCGTCCCACGCCATCCCGCATCTGACCACCA |
| | auxotrophic marker) | GCGAAATGGATTTTTGCATCGAGCTGGGTAATAAGCGTTGGCAATT |
| | | TAACCGCCAGTCAGGCTTTCTTTCACAGATGTGGATTGGCGATAAA |
| | | AAACAACTGCTGACGCCGCTGCGCGATCAGTTCACCCGTGCACCGC |
| | | TGGATAACGACATTGGCGTAAGTGAAGCGACCCGCATTGACCCTAA |
| | | CGCCTGGGTCGAACGCTGGAAGGCGGCGGGCCATTACCAGGCCGA |
| | | AGCAGCGTTGTTGCAGTGCACGGCAGATACACTTGCTGATGCGGTG |
| | | CTGATTACGACCGCTCACGCGTGGCAGCATCAGGGGAAAACCTTAT |
| | | TTATCAGCCGGAAAACCTACCGGATTGATGGTAGTGGTCAAATGGC |
| | | GATTACCGTTGATGTTGAAGTGGCGAGCGATACACCGCATCCGGCG |
| | | CGGATTGGCCTGAACTGCCAG |
| 51 | Sequence of the 5′- | AAAACCTTTTTTCCTATTCAAACACAAGGCATTGCTTCAACACGTGT |
| | Region used for | GCGTATCCTTAACACAGATACTCCATACTTCTAATAATGTGATAGAC |
| | knock out of | GAATACAAAGATGTTCACTCTGTGTTGTGTCTACAAGCATTTCTTAT |
| | PpOCH1: | TCTGATTGGGGATATTCTAGTTACAGCACTAAACAACTGGCGATAC |
| | | AAACTTAAATTAAATAATCCGAATCTAGAAAATGAACTTTTGGATG |
| | | GTCCGCCTGTTGGTTGGATAAATCAATACCGATTAAATGGATTCTAT |
| | | TCCAATGAGAGAGTAATCCAAGACACTCTGATGTCAATAATCATTT |
| | | GCTTGCAACAACAAACCCGTCATCTAATCAAAGGGTTTGATGAGGC |
| | | TTACCTTCAATTGCAGATAAACTCATTGCTGTCCACTGCTGTATTAT |
| | | GTGAGAATATGGGTGATGAATCTGGTCTTCTCCACTCAGCTAACAT |
| | | GGCTGTTTGGGCAAAGGTGGTACAATTATACGGAGATCAGGCAATA |
| | | GTGAAATTGTTGAATATGGCTACTGGACGATGCTTCAAGGATGTAC |
| | | GTCTAGTAGGAGCCGTGGGAAGATTGCTGGCAGAACCAGTTGGCAC |
| | | GTCGCAACAATCCCCAAGAAATGAAATAAGTGAAAACGTAACGTC |
| | | AAAGACAGCAATGGAGTCAATATTGATAACACCACTGGCAGAGCG |
| | | GTTCGTACGTCGTTTTGGAGCCGATATGAGGCTCAGCGTGCTAACA |
| | | GCACGATTGACAAGAAGACTCTCGAGTGACAGTAGGTTGAGTAAAG |
| | | TATTCGCTTAGATTCCCAACCTTCGTTTTATTCTTTCGTAGACAAAG |
| | | AAGCTGCATGCGAACATAGGGACAACTTTTATAAATCCAATTGTCA |
| | | AACCAACGTAAAACCCTCTGGCACCATTTTCAACATATATTTGTGAA |
| | | GCAGTACGCAATATCGATAAATACTCACCGTTGTTTGTAACAGCCCC |
| | | AACTTGCATACGCCTTCTAATGACCTCAAATGGATAAGCCGCAGCTT |
| | | GTGCTAACATACCAGCAGCACCGCCCGCGGTCAGCTGCGCCCACAC |
| | | ATATAAAGGCAATCTACGATCATGGGAGGAATTAGTTTTGACCGTC |
| | | AGGTCTTCAAGAGTTTTGAACTCTTCTTCTTGAACTGTGTAACCTTTT |
| | | AAATGACGGGATCTAAATACGTCATGGATGAGATCATGTGTGTAAA |
| | | AACTGACTCCAGCATATGGAATCATTCCAAAGATTGTAGGAGCGAA |
| | | CCCACGATAAAAGTTTCCCAACCTTGCCAAAGTGTCTAATGCTGTGA |
| | | CTTGAAATCTGGGTTCCTCGTTGAAGACCCTGCGTACTATGCCCAAA |
| | | AACTTTCCTCCACGAGCCCTATTAACTTCTCTATGAGTTTCAAATGC |
| | | CAAACGGACACGGATTAGGTCCAATGGGTAAGTGAAAAACACAGA |
| | | GCAAACCCCAGCTAATGAGCCGGCCAGTAACCGTCTTGGAGCTGTT |
| | | TCATAAGAGTCATTAGGGATCAATAACGTTCTAATCTGTTCATAACA |
| | | TACAAATTTTATGGCTGCATAGGGAAAAATTCTCAACAGGGTAGCC |
| | | GAATGACCCTGATATAGACCTGCGACACCATCATACCCATAGATCT |
| | | GCCTGACAGCCTTAAAGAGCCCGCTAAAAGACCCGGAAAACCGAG |
| | | AGAACTCTGGATTAGCAGTCTGAAAAAGAATCTTCACTCTGTCTAG |
| | | TGGAGCAATTAATGTCTTAGCGGCACTTCCTGCTACTCCGCCAGCTA |
| | | CTCCTGAATAGATCACATACTGCAAAGACTGCTTGTCGATGACCTTG |
| | | GGGTTATTTAGCTTCAAGGGCAATTTTTGGGACATTTTGGACACAGG |
| | | AGACTCAGAAACAGACACAGAGCGTTCTGAGTCCTGGTGCTCCTGA |
| | | CGTAGGCCTAGAACAGGAATTATTGGCTTTATTTGTTTGTCCATTTC |
| | | ATAGGCTTGGGGTAATAGATAGATGACAGAGAAATAGAGAAGACC |
| | | TAATATTTTTTGTTCATGGCAAATCGCGGGTTCGCGGTCGGGTCACA |
| | | CACGGAGAAGTAATGAGAAGAGCTGGTAATCTGGGGTAAAAGGGT |
| | | TCAAAAGAAGGTCGCCTGGTAGGGATGCAATACAAGGTTGTCTTGG |
| | | AGTTTACATTGACCAGATGATTTGGCTTTTTCTCTGTTCAATTCACAT |
| | | TTTTCAGCGAGAATCGGATTGACGGAGAAATGGCGGGGTGTGGGGT |
| | | GGATAGATGGCAGAAATGCTCGCAATCACCGCGAAAGAAAGACTTT |
| | | ATGGAATAGAACTACTGGGTGGTGTAAGGATTACATAGCTAGTCCA |
| | | ATGGAGTCCGTTGGAAAGGTAAGAAGAAGCTAAAACCGGCTAAGT |
| | | AACTAGGGAAGAATGATCAGACTTTGATTTGATGAGGTCTGAAAAT |
| | | ACTCTGCTGCTTTTTCAGTTGCTTTTTCCCTGCAACCTATCATTTTCC |
| | | TTTTCATAAGCCTGCCTTTTCTGTTTTCACTTATATGAGTTCCGCCGA |
| | | GACTTCCCCAAATTCTCTCCTGGAACATTCTCTATCGCTCTCCTTCCA |
| | | AGTTGCGCCCCCTGGCACTGCCTAGTAATATTACCACGCGACTTATA |
| | | TTCAGTTCCACAATTTCCAGTGTTCGTAGCAAATATCATCAGCCATG |
| | | GCGAAGGCAGATGGCAGTTTGCTCTACTATAATCCTCACAATCCAC |
| | | CCAGAAGGTATTACTTCTACATGGCTATATTCGCCGTTTCTGTCATT |
| | | TGCGTTTTGTACGGACCCTCACAACAATTATCATCTCCAAAAATAGA |
| | | CTATGATCCATTGACGCTCCGATCACTTGATTTGAAGACTTTGGAAG |
| | | CTCCTTCACAGTTGAGTCCAGGCACCGTAGAAGATAATCTTCG |
| 52 | Sequence of the 3′- | AAAGCTAGAGTAAAATAGATATAGCGAGATTAGAGAATGAATACC |
| | Region used for | TTCTTCTAAGCGATCGTCCGTCATCATAGAATATCATGGACTGTATA |
| | knock out of | GTTTTTTTTTTGTACATATAATGATTAAACGGTCATCCAACATCTCGT |
| | PpOCH1: | TGACAGATCTCTCAGTACGCGAAATCCCTGACTATCAAAGCAAGAA |
| | | CCGATGAAGAAAAAAACAACAGTAACCCAAACACCACAACAAACA |
| | | CTTTATCTTCTCCCCCCCAACACCAATCATCAAAGAGATGTCGGAAC |
| | | CAAACACCAAGAAGCAAAAACTAACCCCATATAAAAACATCCTGGT |
| | | AGATAATGCTGGTAACCCGCTCTCCTTCCATATTCTGGGCTACTTCA |
| | | CGAAGTCTGACCGGTCTCAGTTGATCAACATGATCCTCGAAATGGG |
| | | TGGCAAGATCGTTCCAGACCTGCCTCCTCTGGTAGATGGAGTGTTGT |
| | | TTTTGACAGGGGATTACAAGTCTATTGATGAAGATACCCTAAAGCA |
| | | ACTGGGGGACGTTCCAATATACAGAGACTCCTTCATCTACCAGTGTT |
| | | TTGTGCACAAGACATCTCTTCCCATTGACACTTTCCGAATTGACAAG |
| | | AACGTCGACTTGGCTCAAGATTTGATCAATAGGGCCCTTCAAGAGT |
| | | CTGTGGATCATGTCACTTCTGCCAGCACAGCTGCAGCTGCTGCTGTT |
| | | GTTGTCGCTACCAACGGCCTGTCTTCTAAACCAGACGCTCGTACTAG |
| | | CAAAATACAGTTCACTCCCGAAGAAGATCGTTTTATTCTTGACTTTG |
| | | TTAGGAGAAATCCTAAACGAAGAAACACACATCAACTGTACACTGA |
| | | GCTCGCTCAGCACATGAAAAACCATACGAATCATTCTATCCGCCAC |
| | | AGATTTCGTCGTAATCTTTCCGCTCAACTTGATTGGGTTTATGATAT |
| | | CGATCCATTGACCAACCAACCTCGAAAAGATGAAAACGGGAACTAC |
| | | ATCAAGGTACAAGGCCTTCCA |
| 53 | K. lactis UDP- | AAACGTAACGCCTGGCACTCTATTTTCTCAAACTTCTGGGACGGAA |
| | GlcNAc transporter | GAGCTAAATATTGTGTTGCTTGAACAAACCCAAAAAAACAAAAAAA |
| | gene (KIMNN2-2) | TGAACAAACTAAAACTACACCTAAATAAACCGTGTGTAAAACGTAG |
| | ORF underlined | TACCATATTACTAGAAAAGATCACAAGTGTATCACACATGTGCATC |
| | | TCATATTACATCTTTTATCCAATCCATTCTCTCTATCCCGTCTGTTCC |
| | | TGTCAGATTCTTTTTCCATAAAAAGAAGAAGACCCCGAATCTCACC |
| | | GGTACAATGCAAAACTGCTGAAAAAAAAAGAAAGTTCACTGGATA |
| | | CGGGAACAGTGCCAGTAGGCTTCACCACATGGACAAAACAATTGAC |
| | | GATAAAATAAGCAGGTGAGCTTCTTTTTCAAGTCACGATCCCTTTAT |
| | | GTCTCAGAAACAATATATACAAGCTAAACCCTTTTGAACCAGTTCTC |
| | | TCTTCATAGTTATGTTCACATAAATTGCGGGAACAAGACTCCGCTGG |
| | | CTGTCAGGTACACGTTGTAACGTTTTCGTCCGCCCAATTATTAGCAC |
| | | AACATTGGCAAAAAGAAAAACTGCTCGTTTTCTCTACAGGTAAATT |
| | | ACAATTTTTTTCAGTAATTTTCGCTGAAAAATTTAAAGGGCAGGAA |
| | | AAAAAGACGATCTCGACTTTGCATAGATGCAAGAACTGTGGTCAAA |
| | | ACTTGAAATAGTAATTTTGCTGTGCGTGAACTAATAAATATATATAT |
| | | ATATATATATATATATTTGTGTATTTTGTATATGTAATTGTGCACGTC |
| | | TTGGCTATTGGATATAAGATTTTCGCGGGTTGATGACATAGAGCGT |
| | | GTACTACTGTAATAGTTGTATATTCAAAAGCTGCTGCGTGGAGAAA |
| | | GACTAAAATAGATAAAAAGCACACATTTTGACTTCGGTACCGTCAA |
| | | CTTAGTGGGACAGTCTTTTATATTTGGTGTAAGCTCATTTCTGGTAC |
| | | TATTCGAAACAGAACAGTGTTTTCTGTATTACCGTCCAATCGTTTGT |
| | | CATGAGTTTTGTATTGATTTTGTCGTTAGTGTTCGGAGGATGTTGTT |
| | | CCAATGTGATTAGTTTCGAGCACATGGTGCAAGGCAGCAATATAAA |
| | | TTTGGGAAATATTGTTACATTCACTCAATTCGTGTCTGTGACGCTAA |
| | | TTCAGTTGCCCAATGCTTTGGACTTCTCTCACTTTCCGTTTAGGTTGC |
| | | GACCTAGACACATTCCTCTTAAGATCCATATGTTAGCTGTGTTTTTG |
| | | TTCTTTACCAGTTCAGTCGCCAATAACAGTGTGTTTAAATTTGACAT |
| | | TTCCGTTCCGATTCATATTATCATTAGATTTTCAGGTACCACTTTGAC |
| | | GATGATAATAGGTTGGGCTGTTTGTAATAAGAGGTACTCCAAACTT |
| | | CAGGTGCAATCTGCCATCATTATGACGCTTGGTGCGATTGTCGCATC |
| | | ATTATACCGTGACAAAGAATTTTCAATGGACAGTTTAAAGTTGAAT |
| | | ACGGATTCAGTGGGTATGACCCAAAAATCTATGTTTGGTATCTTTGT |
| | | TGTGCTAGTGGCCACTGCCTTGATGTCATTGTTGTCGTTGCTCAACG |
| | | AATGGACGTATAACAAGTACGGGAAACATTGGAAAGAAACTTTGTT |
| | | CTATTCGCATTTCTTGGCTCTACCGTTGTTTATGTTGGGGTACACAA |
| | | GGCTCAGAGACGAATTCAGAGACCTCTTAATTTCCTCAGACTCAAT |
| | | GGATATTCCTATTGTTAAATTACCAATTGCTACGAAACTTTTCATGC |
| | | TAATAGCAAATAACGTGACCCAGTTCATTTGTATCAAAGGTGTTAA |
| | | CATGCTAGCTAGTAACACGGATGCTTTGACACTTTCTGTCGTGCTTC |
| | | TAGTGCGTAAATTTGTTAGTCTTTTACTCAGTGTCTACATCTACAAG |
| | | AACGTCCTATCCGTGACTGCATACCTAGGGACCATCACCGTGTTCCT |
| | | GGGAGCTGGTTTGTATTCATATGGTTCGGTCAAAACTGCACTGCCTC |
| | | GCTGAAACAATCCACGTCTGTATGATACTCGTTTCAGAATTTTTTTG |
| | | ATTTTCTGCCGGATATGGTTTCTCATCTTTACAATCGCATTCTTAATT |
| | | ATACCAGAACGTAATTCAATGATCCCAGTGACTCGTAACTCTTATAT |
| | | GTCAATTTAAGC |
| 54 | Sequence of the 5′- | GGCCGAGCGGGCCTAGATTTTCACTACAAATTTCAAAACTACGCGG |
| | Region used for | ATTTATTGTCTCAGAGAGCAATTTGGCATTTCTGAGCGTAGCAGGA |
| | knock out of | GGCTTCATAAGATTGTATAGGACCGTACCAACAAATTGCCGAGGCA |
| | PpBMT2: | CAACACGGTATGCTGTGCACTTATGTGGCTACTTCCCTACAACGGAA |
| | | TGAAACCTTCCTCTTTCCGCTTAAACGAGAAAGTGTGTCGCAATTGA |
| | | ATGCAGGTGCCTGTGCGCCTTGGTGTATTGTTTTTGAGGGCCCAATT |
| | | TATCAGGCGCCTTTTTTCTTGGTTGTTTTCCCTTAGCCTCAAGCAAGG |
| | | TTGGTCTATTTCATCTCCGCTTCTATACCGTGCCTGATACTGTTGGAT |
| | | GAGAACACGACTCAACTTCCTGCTGCTCTGTATTGCCAGTGTTTTGT |
| | | CTGTGATTTGGATCGGAGTCCTCCTTACTTGGAATGATAATAATCTT |
| | | GGCGGAATCTCCCTAAACGGAGGCAAGGATTCTGCCTATGATGATC |
| | | TGCTATCATTGGGAAGCTTCAACGACATGGAGGTCGACTCCTATGT |
| | | CACCAACATCTACGACAATGCTCCAGTGCTAGGATGTACGGATTTG |
| | | TCTTATCATGGATTGTTGAAAGTCACCCCAAAGCATGACTTAGCTTG |
| | | CGATTTGGAGTTCATAAGAGCTCAGATTTTGGACATTGACGTTTACT |
| | | CCGCCATAAAAGACTTAGAAGATAAAGCCTTGACTGTAAAACAAAA |
| | | GGTTGAAAAACACTGGTTTACGTTTTATGGTAGTTCAGTCTTTCTGC |
| | | CCGAACACGATGTGCATTACCTGGTTAGACGAGTCATCTTTTCGGCT |
| | | GAAGGAAAGGCGAACTCTCCAGTAACATC |
| 55 | Sequence of the 3′- | CCATATGATGGGTGTTTGCTCACTCGTATGGATCAAAATTCCATGGT |
| | Region used for | TTCTTCTGTACAACTTGTACACTTATTTGGACTTTTCTAACGGTTTTT |
| | knock out of | CTGGTGATTTGAGAAGTCCTTATTTTGGTGTTCGCAGCTTATCCGTG |
| | PpBMT2: | ATTGAACCATCAGAAATACTGCAGCTCGTTATCTAGTTTCAGAATGT |
| | | GTTGTAGAATACAATCAATTCTGAGTCTAGTTTGGGTGGGTCTTGGC |
| | | GACGGGACCGTTATATGCATCTATGCAGTGTTAAGGTACATAGAAT |
| | | GAAAATGTAGGGGTTAATCGAAAGCATCGTTAATTTCAGTAGAACG |
| | | TAGTTCTATTCCCTACCCAAATAATTTGCCAAGAATGCTTCGTATCC |
| | | ACATACGCAGTGGACGTAGCAAATTTCACTTTGGACTGTGACCTCA |
| | | AGTCGTTATCTTCTACTTGGACATTGATGGTCATTACGTAATCCACA |
| | | AAGAATTGGATAGCCTCTCGTTTTATCTAGTGCACAGCCTAATAGCA |
| | | CTTAAGTAAGAGCAATGGACAAATTTGCATAGACATTGAGCTAGAT |
| | | ACGTAACTCAGATCTTGTTCACTCATGGTGTACTCGAAGTACTGCTG |
| | | GAACCGTTACCTCTTATCATTTCGCTACTGGCTCGTGAAACTACTGG |
| | | ATGAAAAAAAAAAAAGAGCTGAAAGCGAGATCATCCCATTTTGTCA |
| | | TCATACAAATTCACGCTTGCAGTTTTGCTTCGTTAACAAGACAAGAT |
| | | GTCTTTATCAAAGACCCGTTTTTTCTTCTTGAAGAATACTTCCCTGTT |
| | | GAGCACATGCAAACCATATTTATCTCAGATTTCACTCAACTTGGGTG |
| | | CTTCCAAGAGAAGTAAAATTCTTCCCACTGCATCAACTTCCAAGAA |
| | | ACCCGTAGACCAGTTTCTCTTCAGCCAAAAGAAGTTGCTCGCCGATC |
| | | ACCGCGGTAACAGAGGAGTCAGAAGGTTTCACACCCTTCCATCCCG |
| | | ATTTCAAAGTCAAAGTGCTGCGTTGAACCAAGGTTTTCAGGTTGCC |
| | | AAAGCCCAGTCTGCAAAAACTAGTTCCAAATGGCCTATTAATTCCC |
| | | ATAAAAGTGTTGGCTACGTATGTATCGGTACCTCCATTCTGGTATTT |
| | | GCTATTGTTGTCGTTGGTGGGTTGACTAGACTGACCGAATCCGGTCT |
| | | TTCCATAACGGAGTGGAAACCTATCACTGGTTCGGTTCCCCCACTGA |
| | | CTGAGGAAGACTGGAAGTTGGAATTTGAAAAATACAAACAAAGCC |
| | | CTGAGTTTCAGGAACTAAATTCTCACATAACATTGGAAGAGTTCAA |
| | | GTTTATATTTTCCATGGAATGGGGACATAGATTGTTGGGAAGGGTC |
| | | ATCGGCCTGTCGTTTGTTCTTCCCACGTTTTACTTCATTGCCCGTCGA |
| | | AAGTGTTCCAAAGATGTTGCATTGAAACTGCTTGCAATATGCTCTAT |
| | | GATAGGATTCCAAGGTTTCATCGGCTGGTGGATGGTGTATTCCGGA |
| | | TTGGACAAACAGCAATTGGCTGAACGTAACTCCAAACCAACTGTGT |
| | | CTCCATATCGCTTAACTACCCATCTTGGAACTGCATTTGTTATTTACT |
| | | GTTACATGATTTACACAGGGCTTCAAGTTTTGAAGAACTATAAGAT |
| | | CATGAAACAGCCTGAAGCGTATGTTCAAATTTTCAAGCAAATTGCG |
| | | TCTCCAAAATTGAAAACTTTCAAGAGACTCTCTTCAGTTCTATTAGG |
| | | CCTGGTG |
| 56 | DNA encodes | ATGTCTGCCAACCTAAAATATCTTTCCTTGGGAATTTTGGTGTTTCA |
| | MmSLC35A3 UDP- | GACTACCAGTCTGGTTCTAACGATGCGGTATTCTAGGACTTTAAAA |
| | GlcNAc transporter | GAGGAGGGGCCTCGTTATCTGTCTTCTACAGCAGTGGTTGTGGCTG |
| | | AATTTTTGAAGATAATGGCCTGCATCTTTTTAGTCTACAAAGACAGT |
| | | AAGTGTAGTGTGAGAGCACTGAATAGAGTACTGCATGATGAAATTC |
| | | TTAATAAGCCCATGGAAACCCTGAAGCTCGCTATCCCGTCAGGGAT |
| | | ATATACTCTTCAGAACAACTTACTCTATGTGGCACTGTCAAACCTAG |
| | | ATGCAGCCACTTACCAGGTTACATATCAGTTGAAAATACTTACAAC |
| | | AGCATTATTTTCTGTGTCTATGCTTGGTAAAAAATTAGGTGTGTACC |
| | | AGTGGCTCTCCCTAGTAATTCTGATGGCAGGAGTTGCTTTTGTACAG |
| | | TGGCCTTCAGATTCTCAAGAGCTGAACTCTAAGGACCTTTCAACAG |
| | | GCTCACAGTTTGTAGGCCTCATGGCAGTTCTCACAGCCTGTTTTTCA |
| | | AGTGGCTTTGCTGGAGTTTATTTTGAGAAAATCTTAAAAGAAACAA |
| | | AACAGTCAGTATGGATAAGGAACATTCAACTTGGTTTCTTTGGAAG |
| | | TATATTTGGATTAATGGGTGTATACGTTTATGATGGAGAATTGGTCT |
| | | CAAAGAATGGATTTTTTCAGGGATATAATCAACTGACGTGGATAGT |
| | | TGTTGCTCTGCAGGCACTTGGAGGCCTTGTAATAGCTGCTGTCATCA |
| | | AATATGCAGATAACATTTTAAAAGGATTTGCGACCTCCTTATCCATA |
| | | ATATTGTCAACAATAATATCTTATTTTTGGTTGCAAGATTTTGTGCC |
| | | AACCAGTGTCTTTTTCCTTGGAGCCATCCTTGTAATAGCAGCTACTT |
| | | TCTTGTATGGTTACGATCCCAAACCTGCAGGAAATCCCACTAAAGC |
| | | ATAG |
| 57 | PpGAPDH promoter | TTTTTGTAGAAATGTCTTGGTGTCCTCGTCCAATCAGGTAGCCATCT |
| | | CTGAAATATCTGGCTCCGTTGCAACTCCGAACGACCTGCTGGCAAC |
| | | GTAAAATTCTCCGGGGTAAAACTTAAATGTGGAGTAATGGAACCAG |
| | | AAACGTCTCTTCCCTTCTCTCTCCTTCCACCGCCCGTTACCGTCCCTA |
| | | GGAAATTTTACTCTGCTGGAGAGCTTCTTCTACGGCCCCCTTGCAGC |
| | | AATGCTCTTCCCAGCATTACGTTGCGGGTAAAACGGAGGTCGTGTA |
| | | CCCGACCTAGCAGCCCAGGGATGGAAAAGTCCCGGCCGTCGCTGGC |
| | | AATAATAGCGGGCGGACGCATGTCATGAGATTATTGGAAACCACCA |
| | | GAATCGAATATAAAAGGCGAACACCTTTCCCAATTTTGGTTTCTCCT |
| | | GACCCAAAGACTTTAAATTTAATTTATTTGTCCCTATTTCAATCAAT |
| | | TGAACAACTATCAAAACACA |
| 58 | ScCYC TT | ACAGGCCCCTTTTCCTTTGTCGATATCATGTAATTAGTTATGTCACG |
| | | CTTACATTCACGCCCTCCTCCCACATCCGCTCTAACCGAAAAGGAAG |
| | | GAGTTAGACAACCTGAAGTCTAGGTCCCTATTTATTTTTTTTAATAG |
| | | TTATGTTAGTATTAAGAACGTTATTTATATTTCAAATTTTTCTTTTTT |
| | | TTCTGTACAAACGCGTGTACGCATGTAACATTATACTGAAAACCTTG |
| | | CTTGAGAAGGTTTTGGGACGCTCGAAGGCTTTAATTTGCAAGCTGC |
| | | CGGCTCTTAAG |
| 59 | Sequence of the 5′- | GATCTGGCCATTGTGAAACTTGACACTAAAGACAAAACTCTTAGAG |
| | Region used for | TTTCCAATCACTTAGGAGACGATGTTTCCTACAACGAGTACGATCCC |
| | knock out of | TCATTGATCATGAGCAATTTGTATGTGAAAAAAGTCATCGACCTTG |
| | PpMNN4L1: | ACACCTTGGATAAAAGGGCTGGAGGAGGTGGAACCACCTGTGCAG |
| | | GCGGTCTGAAAGTGTTCAAGTACGGATCTACTACCAAATATACATC |
| | | TGGTAACCTGAACGGCGTCAGGTTAGTATACTGGAACGAAGGAAAG |
| | | TTGCAAAGCTCCAAATTTGTGGTTCGATCCTCTAATTACTCTCAAAA |
| | | GCTTGGAGGAAACAGCAACGCCGAATCAATTGACAACAATGGTGTG |
| | | GGTTTTGCCTCAGCTGGAGACTCAGGCGCATGGATTCTTTCCAAGCT |
| | | ACAAGATGTTAGGGAGTACCAGTCATTCACTGAAAAGCTAGGTGAA |
| | | GCTACGATGAGCATTTTCGATTTCCACGGTCTTAAACAGGAGACTTC |
| | | TACTACAGGGCTTGGGGTAGTTGGTATGATTCATTCTTACGACGGTG |
| | | AGTTCAAACAGTTTGGTTTGTTCACTCCAATGACATCTATTCTACAA |
| | | AGACTTCAACGAGTGACCAATGTAGAATGGTGTGTAGCGGGTTGCG |
| | | AAGATGGGGATGTGGACACTGAAGGAGAACACGAATTGAGTGATT |
| | | TGGAACAACTGCATATGCATAGTGATTCCGACTAGTCAGGCAAGAG |
| | | AGAGCCCTCAAATTTACCTCTCTGCCCCTCCTCACTCCTTTTGGTAC |
| | | GCATAATTGCAGTATAAAGAACTTGCTGCCAGCCAGTAATCTTATTT |
| | | CATACGCAGTTCTATATAGCACATAATCTTGCTTGTATGTATGAAAT |
| | | TTACCGCGTTTTAGTTGAAATTGTTTATGTTGTGTGCCTTGCATGAA |
| | | ATCTCTCGTTAGCCCTATCCTTACATTTAACTGGTCTCAAAACCTCT |
| | | ACCAATTCCATTGCTGTACAACAATATGAGGCGGCATTACTGTAGG |
| | | GTTGGAAAAAAATTGTCATTCCAGCTAGAGATCACACGACTTCATC |
| | | ACGCTTATTGCTCCTCATTGCTAAATCATTTACTCTTGACTTCGACCC |
| | | AGAAAAGTTCGCC |
| 60 | Sequence of the 3′- | GCATGTCAAACTTGAACACAACGACTAGATAGTTGTTTTTTCTATAT |
| | Region used for | AAAACGAAACGTTATCATCTTTAATAATCATTGAGGTTTACCCTTAT |
| | knock out of | AGTTCCGTATTTTCGTTTCCAAACTTAGTAATCTTTTGGAAATATCAT |
| | PpMNN4L1: | CAAAGCTGGTGCCAATCTTCTTGTTTGAAGTTTCAAACTGCTCCACC |
| | | AAGCTACTTAGAGACTGTTCTAGGTCTGAAGCAACTTCGAACACAG |
| | | AGACAGCTGCCGCCGATTGTTCTTTTTTGTGTTTTTCTTCTGGAAGA |
| | | GGGGCATCATCTTGTATGTCCAATGCCCGTATCCTTTCTGAGTTGTC |
| | | CGACACATTGTCCTTCGAAGAGTTTCCTGACATTGGGCTTCTTCTAT |
| | | CCGTGTATTAATTTTGGGTTAAGTTCCTCGTTTGCATAGCAGTGGAT |
| | | ACCTCGATTTTTTTGGCTCCTATTTACCTGACATAATATTCTACTATA |
| | | ATCCAACTTGGACGCGTCATCTATGATAACTAGGCTCTCCTTTGTTC |
| | | AAAGGGGACGTCTTCATAATCCACTGGCACGAAGTAAGTCTGCAAC |
| | | GAGGCGGCTTTTGCAACAGAACGATAGTGTCGTTTCGTACTTGGAC |
| | | TATGCTAAACAAAAGGATCTGTCAAACATTTCAACCGTGTTTCAAG |
| | | GCACTCTTTACGAATTATCGACCAAGACCTTCCTAGACGAACATTTC |
| | | AACATATCCAGGCTACTGCTTCAAGGTGGTGCAAATGATAAAGGTA |
| | | TAGATATTAGATGTGTTTGGGACCTAAAACAGTTCTTGCCTGAAGAT |
| | | TCCCTTGAGCAACAGGCTTCAATAGCCAAGTTAGAGAAGCAGTACC |
| | | AAATCGGTAACAAAAGGGGGAAGCATATAAAACCTTTACTATTGCG |
| | | ACAAAATCCATCCTTGAAAGTAAAGCTGTTTGTTCAATGTAAAGCA |
| | | TACGAAACGAAGGAGGTAGATCCTAAGATGGTTAGAGAACTTAAC |
| | | GGGACATACTCCAGCTGCATCCCATATTACGATCGCTGGAAGACTTT |
| | | TTTCATGTACGTATCGCCCACCAACCTTTCAAAGCAAGCTAGGTATG |
| | | ATTTTGACAGTTCTCACAATCCATTGGTTTTCATGCAACTTGAAAAA |
| | | ACCCAACTCAAACTTCATGGGGATCCATACAATGTAAATCATTACG |
| | | AGAGGGCGAGGTTGAAAAGTTTCCATTGCAATCACGTCGCATCATG |
| | | GCTACTGAAAGGCCTTAAC |
| 61 | Sequence of the 5′- | TCATTCTATATGTTCAAGAAAAGGGTAGTGAAAGGAAAGAAAAGG |
| | Region used for | CATATAGGCGAGGGAGAGTTAGCTAGCATACAAGATAATGAAGGA |
| | knock out of | TCAATAGCGGTAGTTAAAGTGCACAAGAAAAGAGCACCTGTTGAGG |
| | PpPNO1 and | CTGATGATAAAGCTCCAATTACATTGCCACAGAGAAACACAGTAAC |
| | PpMNN4: | AGAAATAGGAGGGGATGCACCACGAGAAGAGCATTCAGTGAACAA |
| | | CTTTGCCAAATTCATAACCCCAAGCGCTAATAAGCCAATGTCAAAG |
| | | TCGGCTACTAACATTAATAGTACAACAACTATCGATTTTCAACCAG |
| | | ATGTTTGCAAGGACTACAAACAGACAGGTTACTGCGGATATGGTGA |
| | | CACTTGTAAGTTTTTGCACCTGAGGGATGATTTCAAACAGGGATGG |
| | | AAATTAGATAGGGAGTGGGAAAATGTCCAAAAGAAGAAGCATAAT |
| | | ACTCTCAAAGGGGTTAAGGAGATCCAAATGTTTAATGAAGATGAGC |
| | | TCAAAGATATCCCGTTTAAATGCATTATATGCAAAGGAGATTACAA |
| | | ATCACCCGTGAAAACTTCTTGCAATCATTATTTTTGCGAACAATGTT |
| | | TCCTGCAACGGTCAAGAAGAAAACCAAATTGTATTATATGTGGCAG |
| | | AGACACTTTAGGAGTTGCTTTACCAGCAAAGAAGTTGTCCCAATTTC |
| | | TGGCTAAGATACATAATAATGAAAGTAATAAAGTTTAGTAATTGCA |
| | | TTGCGTTGACTATTGATTGCATTGATGTCGTGTGATACTTTCACCGA |
| | | AAAAAAACACGAAGCGCAATAGGAGCGGTTGCATATTAGTCCCCA |
| | | AAGCTATTTAATTGTGCCTGAAACTGTTTTTTAAGCTCATCAAGCAT |
| | | AATTGTATGCATTGCGACGTAACCAACGTTTAGGCGCAGTTTAATC |
| | | ATAGCCCACTGCTAAGCC |
| 62 | Sequence of the 3′- | CGGAGGAATGCAAATAATAATCTCCTTAATTACCCACTGATAAGCT |
| | Region used for | CAAGAGACGCGGTTTGAAAACGATATAATGAATCATTTGGATTTTA |
| | knock out of | TAATAAACCCTGACAGTTTTTCCACTGTATTGTTTTAACACTCATTG |
| | PpPNO1 and | GAAGCTGTATTGATTCTAAGAAGCTAGAAATCAATACGGCCATACA |
| | PpMNN4: | AAAGATGACATTGAATAAGCACCGGCTTTTTTGATTAGCATATACCT |
| | | TAAAGCATGCATTCATGGCTACATAGTTGTTAAAGGGCTTCTTCCAT |
| | | TATCAGTATAATGAATTACATAATCATGCACTTATATTTGCCCATCT |
| | | CTGTTCTCTCACTCTTGCCTGGGTATATTCTATGAAATTGCGTATAG |
| | | CGTGTCTCCAGTTGAACCCCAAGCTTGGCGAGTTTGAAGAGAATGC |
| | | TAACCTTGCGTATTCCTTGCTTCAGGAAACATTCAAGGAGAAACAG |
| | | GTCAAGAAGCCAAACATTTTGATCCTTCCCGAGTTAGCATTGACTG |
| | | GCTACAATTTTCAAAGCCAGCAGCGGATAGAGCCTTTTTTGGAGGA |
| | | AACAACCAAGGGAGCTAGTACCCAATGGGCTCAAAAAGTATCCAA |
| | | GACGTGGGATTGCTTTACTTTAATAGGATACCCAGAAAAAAGTTTA |
| | | GAGAGCCCTCCCCGTATTTACAACAGTGCGGTACTTGTATCGCCTCA |
| | | GGGAAAAGTAATGAACAACTACAGAAAGTCCTTCTTGTATGAAGCT |
| | | GATGAACATTGGGGATGTTCGGAATCTTCTGATGGGTTTCAAACAG |
| | | TAGATTTATTAATTGAAGGAAAGACTGTAAAGACATCATTTGGAAT |
| | | TTGCATGGATTTGAATCCTTATAAATTTGAAGCTCCATTCACAGACT |
| | | TCGAGTTCAGTGGCCATTGCTTGAAAACCGGTACAAGACTCATTTTG |
| | | TGCCCAATGGCCTGGTTGTCCCCTCTATCGCCTTCCATTAAAAAGGA |
| | | TCTTAGTGATATAGAGAAAAGCAGACTTCAAAAGTTCTACCTTGAA |
| | | AAAATAGATACCCCGGAATTTGACGTTAATTACGAATTGAAAAAAG |
| | | ATGAAGTATTGCCCACCCGTATGAATGAAACGTTGGAAACAATTGA |
| | | CTTTGAGCCTTCAAAACCGGACTACTCTAATATAAATTATTGGATAC |
| | | TAAGGTTTTTTCCCTTTCTGACTCATGTCTATAAACGAGATGTGCTC |
| | | AAAGAGAATGCAGTTGCAGTCTTATGCAACCGAGTTGGCATTGAGA |
| | | GTGATGTCTTGTACGGAGGATCAACCACGATTCTAAACTTCAATGG |
| | | TAAGTTAGCATCGACACAAGAGGAGCTGGAGTTGTACGGGCAGACT |
| | | AATAGTCTCAACCCCAGTGTGGAAGTATTGGGGGCCCTTGGCATGG |
| | | GTCAACAGGGAATTCTAGTACGAGACATTGAATTAACATAATATAC |
| | | AATATACAATAAACACAAATAAAGAATACAAGCCTGACAAAAATT |
| | | CACAAATTATTGCCTAGACTTGTCGTTATCAGCAGCGACCTTTTTCC |
| | | AATGCTCAATTTCACGATATGCCTTTTCTAGCTCTGCTTTAAGCTTCT |
| | | CATTGGAATTGGCTAACTCGTTGACTGCTTGGTCAGTGATGAGTTTC |
| | | TCCAAGGTCCATTTCTCGATGTTGTTGTTTTCGTTTTCCTTTAATCTC |
| | | TTGATATAATCAACAGCCTTCTTTAATATCTGAGCCTTGTTCGAGTC |
| | | CCCTGTTGGCAACAGAGCGGCCAGTTCCTTTATTCCGTGGTTTATAT |
| | | TTTCTCTTCTACGCCTTTCTACTTCTTTGTGATTCTCTTTACGCATCTT |
| | | ATGCCATTCTTCAGAACCAGTGGCTGGCTTAACCGAATAGCCAGAG |
| | | CCTGAAGAAGCCGCACTAGAAGAAGCAGTGGCATTGTTGACTATGG |
| 63 | DNA encodes | TCAGTCAGTGCTCTTGATGGTGACCCAGCAAGTTTGACCAGAGAAG |
| | human GnTI | TGATTAGATTGGCCCAAGACGCAGAGGTGGAGTTGGAGAGACAAC |
| | catalytic domain | GTGGACTGCTGCAGCAAATCGGAGATGCATTGTCTAGTCAAAGAGG |
| | (NA) | TAGGGTGCCTACCGCAGCTCCTCCAGCACAGCCTAGAGTGCATGTG |
| | Codon-optimized | ACCCCTGCACCAGCTGTGATTCCTATCTTGGTCATCGCCTGTGACAG |
| | | ATCTACTGTTAGAAGATGTCTGGACAAGCTGTTGCATTACAGACCA |
| | | TCTGCTGAGTTGTTCCCTATCATCGTTAGTCAAGACTGTGGTCACGA |
| | | GGAGACTGCCCAAGCCATCGCCTCCTACGGATCTGCTGTCACTCAC |
| | | ATCAGACAGCCTGACCTGTCATCTATTGCTGTGCCACCAGACCACA |
| | | GAAAGTTCCAAGGTTACTACAAGATCGCTAGACACTACAGATGGGC |
| | | ATTGGGTCAAGTCTTCAGACAGTTTAGATTCCCTGCTGCTGTGGTGG |
| | | TGGAGGATGACTTGGAGGTGGCTCCTGACTTCTTTGAGTACTTTAGA |
| | | GCAACCTATCCATTGCTGAAGGCAGACCCATCCCTGTGGTGTGTCTC |
| | | TGCCTGGAATGACAACGGTAAGGAGCAAATGGTGGACGCTTCTAGG |
| | | CCTGAGCTGTTGTACAGAACCGACTTCTTTCCTGGTCTGGGATGGTT |
| | | GCTGTTGGCTGAGTTGTGGGCTGAGTTGGAGCCTAAGTGGCCAAAG |
| | | GCATTCTGGGACGACTGGATGAGAAGACCTGAGCAAAGACAGGGT |
| | | AGAGCCTGTATCAGACCTGAGATCTCAAGAACCATGACCTTTGGTA |
| | | GAAAGGGAGTGTCTCACGGTCAATTCTTTGACCAACACTTGAAGTT |
| | | TATCAAGCTGAACCAGCAATTTGTGCACTTCACCCAACTGGACCTGT |
| | | CTTACTTGCAGAGAGAGGCCTATGACAGAGATTTCCTAGCTAGAGT |
| | | CTACGGAGCTCCTCAACTGCAAGTGGAGAAAGTGAGGACCAATGAC |
| | | AGAAAGGAGTTGGGAGAGGTGAGAGTGCAGTACACTGGTAGGGAC |
| | | TCCTTTAAGGCTTTCGCTAAGGCTCTGGGTGTCATGGATGACCTTAA |
| | | GTCTGGAGTTCCTAGAGCTGGTTACAGAGGTATTGTCACCTTTCAAT |
| | | TCAGAGGTAGAAGAGTCCACTTGGCTCCTCCACCTACTTGGGAGGG |
| | | TTATGATCCTTCTTGGAATTAG |
| 64 | DNA encodes Pp | ATGCCCAGAAAAATATTTAACTACTTCATTTTGACTGTATTCATGGC |
| | SEC12 (10) | AATTCTTGCTATTGTTTTACAATGGTCTATAGAGAATGGACATGGGC |
| | The last 9 | GCGCC |
| | nucleotides are the | |
| | linker containing the | |
| | AscI restriction site | |
| | used for fusion to | |
| | proteins of interest. | |
| 65 | Sequence of the | AAATGCGTACCTCTTCTACGAGATTCAAGCGAATGAGAATAATGTA |
| | PpPMA1 promoter: | ATATGCAAGATCAGAAAGAATGAAAGGAGTTGAAAAAAAAAACCG |
| | | TTGCGTTTTGACCTTGAATGGGGTGGAGGTTTCCATTCAAAGTAAAG |
| | | CCTGTGTCTTGGTATTTTCGGCGGCACAAGAAATCGTAATTTTCATC |
| | | TTCTAAACGATGAAGATCGCAGCCCAACCTGTATGTAGTTAACCGG |
| | | TCGGAATTATAAGAAAGATTTTCGATCAACAAACCCTAGCAAATAG |
| | | AAAGCAGGGTTACAACTTTAAACCGAAGTCACAAACGATAAACCAC |
| | | TCAGCTCCCACCCAAATTCATTCCCACTAGCAGAAAGGAATTATTTA |
| | | ATCCCTCAGGAAACCTCGATGATTCTCCCGTTCTTCCATGGGCGGGT |
| | | ATCGCAAAATGAGGAATTTTTCAAATTTCTCTATTGTCAAGACTGTT |
| | | TATTATCTAAGAAATAGCCCAATCCGAAGCTCAGTTTTGAAAAAAT |
| | | CACTTCCGCGTTTCTTTTTTACAGCCCGATGAATATCCAAATTTGGA |
| | | ATATGGATTACTCTATCGGGACTGCAGATAATATGACAACAACGCA |
| | | GATTACATTTTAGGTAAGGCATAAACACCAGCCAGAAATGAAACGC |
| | | CCACTAGCCATGGTCGAATAGTCCAATGAATTCAGATAGCTATGGT |
| | | CTAAAAGCTGATGTTTTTTATTGGGTAATGGCGAAGAGTCCAGTAC |
| | | GACTTCCAGCAGAGCTGAGATGGCCATTTTTGGGGGTATTAGTAAC |
| | | TTTTTGAGCTCTTTTCACTTCGATGAAGTGTCCCATTCGGGATATAA |
| | | TCGGATCGCGTCGTTTTCTCGAAAATACAGCTTAGCGTCGTCCGCTT |
| | | GTTGTAAAAGCAGCACCACATTCCTAATCTCTTATATAAACAAAAC |
| | | AACCCAAATTATCAGTGCTGTTTTCCCACCAGATATAAGTTTCTTTT |
| | | CTCTTCCGCTTTTTGATTTTTTATCTCTTTCCTTTAAAAACTTCTTTAC |
| | | CTTAAAGGGCGGCC |
| 66 | Sequence of the | TAAGCTTCACGATTTGTGTTCCAGTTTATCCCCCCTTTATATACCGTT |
| | PpPMA1 terminator: | AACCCTTTCCCTGTTGAGCTGACTGTTGTTGTATTACCGCAATTTTTC |
| | | CAAGTTTGCCATGCTTTTCGTGTTATTTGACCGATGTCTTTTTTCCCA |
| | | AATCAAACTATATTTGTTACCATTTAAACCAAGTTATCTTTTGTATT |
| | | AAGAGTCTAAGTTTGTTCCCAGGCTTCATGTGAGAGTGATAACCAT |
| | | CCAGACTATGATTCTTGTTTTTTATTGGGTTTGTTTGTGTGATACATC |
| | | TGAGTTGTGATTCGTAAAGTATGTCAGTCTATCTAGATTTTTAATAG |
| | | TTAATTGGTAATCAATGACTTGTTTGTTTTAACTTTTAAATTGTGGGT |
| | | CGTATCCACGCGTTTAGTATAGCTGTTCATGGCTGTTAGAGGAGGG |
| | | CGATGTTTATATACAGAGGACAAGAATGAGGAGGCGGCGTGTATTT |
| | | TTAAAATGGAGACGCGACTCCTGTACACCTTATCGGTTGG |
| 67 | Sequence of the | GAAGTAAAGTTGGCGAAACTTTGGGAACCTTTGGTTAAAACTTTGT |
| | PpSEC4 promoter: | AATTTTTGTCGCTACCCATTAGGCAGAATCTGCATCTTGGGAGGGG |
| | | GATGTGGTGGCGTTCTGAGATGTACGCGAAGAATGAAGAGCCAGTG |
| | | GTAACAACAGGCCTAGAGAGATACGGGCATAATGGGTATAACCTAC |
| | | AAGTTAAGAATGTAGCAGCCCTGGAAACCAGATTGAAACGAAAAA |
| | | CGAAATCATTTAAACTGTAGGATGTTTTGGCTCATTGTCTGGAAGGC |
| | | TGGCTGTTTATTGCCCTGTTCTTTGCATGGGAATAAGCTATTATATC |
| | | CCTCACATAATCCCAGAAAATAGATTGAAGCAACGCGAAATCCTTA |
| | | CGTATCGAAGTAGCCTTCTTACACATTCACGTTGTACGGATAAGAA |
| | | AACTACTCAAACGAACAATC |
| 68 | Sequence of the | AATAGATATAGCGAGATTAGAGAATGAATACCTTCTTCTAAGCGAT |
| | PpOCH1 terminator: | CGTCCGTCATCATAGAATATCATGGACTGTATAGTTTTTTTTTTGTAC |
| | | ATATAATGATTAAACGGTCATCCAACATCTCGTTGACAGATCTCTCA |
| | | GTACGCGAAATCCCTGACTATCAAAGCAAGAACCGATGAAGAAAA |
| | | AAACAACAGTAACCCAAACACCACAACAAACACTTTATCTTCTCCC |
| | | CCCCAACACCAATCATCAAAGAGATGTCGGAACACAAACACCAAG |
| | | AAGCAAAAACTAACCCCATATAAAAACATCCTGGTAGATAATGCTG |
| | | GTAACCCGCTCTCCTTCCATATTCTGGGCTACTTCACGAAGTCTGAC |
| | | CGGTCTCAGTTGATCAACATGATCCTCGAAATGG |
| 69 | DNA encodes Mm | GAGCCCGCTGACGCCACCATCCGTGAGAAGAGGGCAAAGATCAAA |
| | ManI catalytic | GAGATGATGACCCATGCTTGGAATAATTATAAACGCTATGCGTGGG |
| | domain (FB) | GCTTGAACGAACTGAAACCTATATCAAAAGAAGGCCATTCAAGCAG |
| | | TTTGTTTGGCAACATCAAAGGAGCTACAATAGTAGATGCCCTGGAT |
| | | ACCCTTTTCATTATGGGCATGAAGACTGAATTTCAAGAAGCTAAAT |
| | | CGTGGATTAAAAAATATTTAGATTTTAATGTGAATGCTGAAGTTTCT |
| | | GTTTTTGAAGTCAACATACGCTTCGTCGGTGGACTGCTGTCAGCCTA |
| | | CTATTTGTCCGGAGAGGAGATATTTCGAAAGAAAGCAGTGGAACTT |
| | | GGGGTAAAATTGCTACCTGCATTTCATACTCCCTCTGGAATACCTTG |
| | | GGCATTGCTGAATATGAAAAGTGGGATCGGGCGGAACTGGCCCTGG |
| | | GCCTCTGGAGGCAGCAGTATCCTGGCCGAATTTGGAACTCTGCATTT |
| | | AGAGTTTATGCACTTGTCCCACTTATCAGGAGACCCAGTCTTTGCCG |
| | | AAAAGGTTATGAAAATTCGAACAGTGTTGAACAAACTGGACAAACC |
| | | AGAAGGCCTTTATCCTAACTATCTGAACCCCAGTAGTGGACAGTGG |
| | | GGTCAACATCATGTGTCGGTTGGAGGACTTGGAGACAGCTTTTATG |
| | | AATATTTGCTTAAGGCGTGGTTAATGTCTGACAAGACAGATCTCGA |
| | | AGCCAAGAAGATGTATTTTGATGCTGTTCAGGCCATCGAGACTCAC |
| | | TTGATCCGCAAGTCAAGTGGGGGACTAACGTACATCGCAGAGTGGA |
| | | AGGGGGGCCTCCTGGAACACAAGATGGGCCACCTGACGTGCTTTGC |
| | | AGGAGGCATGTTTGCACTTGGGGCAGATGGAGCTCCGGAAGCCCGG |
| | | GCCCAACACTACCTTGAACTCGGAGCTGAAATTGCCCGCACTTGTC |
| | | ATGAATCTTATAATCGTACATATGTGAAGTTGGGACCGGAAGCGTT |
| | | TCGATTTGATGGCGGTGTGGAAGCTATTGCCACGAGGCAAAATGAA |
| | | AAGTATTACATCTTACGGCCCGAGGTCATCGAGACATACATGTACA |
| | | TGTGGCGACTGACTCACGACCCCAAGTACAGGACCTGGGCCTGGGA |
| | | AGCCGTGGAGGCTCTAGAAAGTCACTGCAGAGTGAACGGAGGCTA |
| | | CTCAGGCTTACGGGATGTTTACATTGCCCGTGAGAGTTATGACGAT |
| | | GTCCAGCAAAGTTTCTTCCTGGCAGAGACACTGAAGTATTTGTACTT |
| | | GATATTTTCCGATGATGACCTTCTTCCACTAGAACACTGGATCTTCA |
| | | ACACCGAGGCTCATCCTTTCCCTATACTCCGTGAACAGAAGAAGGA |
| | | AATTGATGGCAAAGAGAAATGA |
| 70 | DNA encodes | ATGAACACTATCCACATAATAAAATTACCGCTTAACTACGCCAACT |
| | ScSEC12 (8) | ACACCTCAATGAAACAAAAAATCTCTAAATTTTTCACCAACTTCATC |
| | The last 9 | CTTATTGTGCTGCTTTCTTACATTTTACAGTTCTCCTATAAGCACAAT |
| | nucleotides are the | TTGCATTCCATGCTTTTCAATTACGCGAAGGACAATTTTCTAACGAA |
| | linker containing the | AAGAGACACCATCTCTTCGCCCTACGTAGTTGATGAAGACTTACATC |
| | AscI restriction site | AAACAACTTTGTTTGGCAACCACGGTACAAAAACATCTGTACCTAG |
| | used for fusion to | CGTAGATTCCATAAAAGTGCATGGCGTGGGGCGCGCC |
| | proteins of interest | |
| 71 | Sequence of the 5′- | GAGTCGGCCAAGAGATGATAACTGTTACTAAGCTTCTCCGTAATTA |
| | region that was used | GTGGTATTTTGTAACTTTTACCAATAATCGTTTATGAATACGGATAT |
| | to knock into the | TTTTCGACCTTATCCAGTGCCAAATCACGTAACTTAATCATGGTTTA |
| | PpADE1 locus: | AATACTCCACTTGAACGATTCATTATTCAGAAAAAAGTCAGGTTGG |
| | | CAGAAACACTTGGGCGCTTTGAAGAGTATAAGAGTATTAAGCATTA |
| | | AACATCTGAACTTTCACCGCCCCAATATACTACTCTAGGAAACTCGA |
| | | AAAATTCCTTTCCATGTGTCATCGCTTCCAACACACTTTGCTGTATC |
| | | CTTCCAAGTATGTCCATTGTGAACACTGATCTGGACGGAATCCTACC |
| | | TTTAATCGCCAAAGGAAAGGTTAGAGACATTTATGCAGTCGATGAG |
| | | AACAACTTGCTGTTCGTCGCAACTGACCGTATCTCCGCTTACGATGT |
| | | GATTATGACAAACGGTATTCCTGATAAGGGAAAGATTTTGACTCAG |
| | | CTCTCAGTTTTCTGGTTTGATTTTTTGGCACCCTACATAAAGAATCAT |
| | | TTGGTTGCTTCTAATGACAAGGAAGTCTTTGCTTTACTACCATCAAA |
| | | ACTGTCTGAAGAAAAaTACAAATCTCAATTAGAGGGACGATCCTTG |
| | | ATAGTAAAAAAGCACAGACTGATACCTTTGGAAGCCATTGTCAGAG |
| | | GTTACATCACTGGAAGTGCATGGAAAGAGTACAAGAACTCAAAAA |
| | | CTGTCCATGGAGTCAAGGTTGAAAACGAGAACCTTCAAGAGAGCGA |
| | | CGCCTTTCCAACTCCGATTTTCACACCTTCAACGAAAGCTGAACAGG |
| | | GTGAACACGATGAAAACATCTCTATTGAACAAGCTGCTGAGATTGT |
| | | AGGTAAAGACATTTGTGAGAAGGTCGCTGTCAAGGCGGTCGAGTTG |
| | | TATTCTGCTGCAAAAAACCTCGCCCTTTTGAAGGGGATCATTATTGC |
| | | TGATACGAAATTCGAATTTGGACTGGACGAAAACAATGAATTGGTA |
| | | CTAGTAGATGAAGTTTTAACTCCAGATTCTTCTAGATTTTGGAATCA |
| | | AAAGACTTACCAAGTGGGTAAATCGCAAGAGAGTTACGATAAGCA |
| | | GTTTCTCAGAGATTGGTTGACGGCCAACGGATTGAATGGCAAAGAG |
| | | GGCGTAGCCATGGATGCAGAAATTGCTATCAAGAGTAAAGAAAAG |
| | | TATATTGAAGCTTATGAAGCAATTACTGGCAAGAAATGGGCTTGA |
| 72 | PpALG3 TT | ATTTACAATTAGTAATATTAAGGTGGTAAAAACATTCGTAGAATTG |
| | | AAATGAATTAATATAGTATGACAATGGTTCATGTCTATAAATCTCCG |
| | | GCTTCGGTACCTTCTCCCCAATTGAATACATTGTCAAAATGAATGGT |
| | | TGAACTATTAGGTTCGCCAGTTTCGTTATTAAGAAAACTGTTAAAAT |
| | | CAAATTCCATATCATCGGTTCCAGTGGGAGGACCAGTTCCATCGCC |
| | | AAAATCCTGTAAGAATCCATTGTCAGAACCTGTAAAGTCAGTTTGA |
| | | GATGAAATTTTTCCGGTCTTTGTTGACTTGGAAGCTTCGTTAAGGTT |
| | | AGGTGAAACAGTTTGATCAACCAGCGGCTCCCGTTTTCGTCGCTTAG |
| | | TAG |
| 73 | Sequence of the 3′- | ATGATTAGTACCCTCCTCGCCTTTTTCAGACATCTGAAATTTCCCTTA |
| | region that was used | TTCTTCCAATTCCATATAAAATCCTATTTAGGTAATTAGTAAACAAT |
| | to knock into the | GATCATAAAGTGAAATCATTCAAGTAACCATTCCGTTTATCGTTGAT |
| | PpADE1 locus: | TTAAAATCAATAACGAATGAATGTCGGTCTGAGTAGTCAATTTGTT |
| | | GCCTTGGAGCTCATTGGCAGGGGGTCTTTTGGCTCAGTATGGAAGG |
| | | TTGAAAGGAAAACAGATGGAAAGTGGTTCGTCAGAAAAGAGGTAT |
| | | CCTACATGAAGATGAATGCCAAAGAGATATCTCAAGTGATAGCTGA |
| | | GTTCAGAATTCTTAGTGAGTTAAGCCATCCCAACATTGTGAAGTACC |
| | | TTCATCACGAACATATTTCTGAGAATAAAACTGTCAATTTATACATG |
| | | GAATACTGTGATGGTGGAGATCTCTCCAAGCTGATTCGAACACATA |
| | | GAAGGAACAAAGAGTACATTTCAGAAGAAAAAATATGGAGTATTTT |
| | | TACGCAGGTTTTATTAGCATTGTATCGTTGTCATTATGGAACTGATT |
| | | TCACGGCTTCAAAGGAGTTTGAATCGCTCAATAAAGGTAATAGACG |
| | | AACCCAGAATCCTTCGTGGGTAGACTCGACAAGAGTTATTATTCAC |
| | | AGGGATATAAAACCCGACAACATCTTTCTGATGAACAATTCAAACC |
| | | TTGTCAAACTGGGAGATTTTGGATTAGCAAAAATTCTGGACCAAGA |
| | | AAACGATTTTGCCAAAACATACGTCGGTACGCCGTATTACATGTCTC |
| | | CTGAAGTGCTGTTGGACCAACCCTACTCACCATTATGTGATATATGG |
| | | TCTCTTGGGTGCGTCATGTATGAGCTATGTGCATTGAGGCCTCCTT |
| 74 | DNA encodes | ATGACAGCTCAGTTACAAAGTGAAAGTACTTCTAAAATTGTTTTGGT |
| | ScGAL10 | TACAGGTGGTGCTGGATACATTGGTTCACACACTGTGGTAGAGCTA |
| | | ATTGAGAATGGATATGACTGTGTTGTTGCTGATAACCTGTCGAATTC |
| | | AACTTATGATTCTGTAGCCAGGTTAGAGGTCTTGACCAAGCATCAC |
| | | ATTCCCTTCTATGAGGTTGATTTGTGTGACCGAAAAGGTCTGGAAA |
| | | AGGTTTTCAAAGAATATAAAATTGATTCGGTAATTCACTTTGCTGGT |
| | | TTAAAGGCTGTAGGTGAATCTACACAAATCCCGCTGAGATACTATC |
| | | ACAATAACATTTTGGGAACTGTCGTTTTATTAGAGTTAATGCAACAA |
| | | TACAACGTTTCCAAATTTGTTTTTTCATCTTCTGCTACTGTCTATGGT |
| | | GATGCTACGAGATTCCCAAATATGATTCCTATCCCAGAAGAATGTC |
| | | CCTTAGGGCCTACTAATCCGTATGGTCATACGAAATACGCCATTGA |
| | | GAATATCTTGAATGATCTTTACAATAGCGACAAAAAAAGTTGGAAG |
| | | TTTGCTATCTTGCGTTATTTTAACCCAATTGGCGCACATCCCTCTGG |
| | | ATTAATCGGAGAAGATCCGCTAGGTATACCAAACAATTTGTTGCCA |
| | | TATATGGCTCAAGTAGCTGTTGGTAGGCGCGAGAAGCTTTACATCTT |
| | | CGGAGACGATTATGATTCCAGAGATGGTACCCCGATCAGGGATTAT |
| | | ATCCACGTAGTTGATCTAGCAAAAGGTCATATTGCAGCCCTGCAAT |
| | | ACCTAGAGGCCTACAATGAAAATGAAGGTTTGTGTCGTGAGTGGAA |
| | | CTTGGGTTCCGGTAAAGGTTCTACAGTTTTTGAAGTTTATCATGCAT |
| | | TCTGCAAAGCTTCTGGTATTGATCTTCCATACAAAGTTACGGGCAGA |
| | | AGAGCAGGTGATGTTTTGAACTTGACGGCTAAACCAGATAGGGCCA |
| | | AACGCGAACTGAAATGGCAGACCGAGTTGCAGGTTGAAGACTCCTG |
| | | CAAGGATTTATGGAAATGGACTACTGAGAATCCTTTTGGTTACCAG |
| | | TTAAGGGGTGTCGAGGCCAGATTTTCCGCTGAAGATATGCGTTATG |
| | | ACGCAAGATTTGTGACTATTGGTGCCGGCACCAGATTTCAAGCCAC |
| | | GTTTGCCAATTTGGGCGCCAGCATTGTTGACCTGAAAGTGAACGGA |
| | | CAATCAGTTGTTCTTGGCTATGAAAATGAGGAAGGGTATTTGAATC |
| | | CTGATAGTGCTTATATAGGCGCCACGATCGGCAGGTATGCTAATCG |
| | | TATTTCGAAGGGTAAGTTTAGTTTATGCAACAAAGACTATCAGTTA |
| | | ACCGTTAATAACGGCGTTAATGCGAATCATAGTAGTATCGGTTCTTT |
| | | CCACAGAAAAAGATTTTTGGGACCCATCATTCAAAATCCTTCAAAG |
| | | GATGTTTTTACCGCCGAGTACATGCTGATAGATAATGAGAAGGACA |
| | | CCGAATTTCCAGGTGATCTATTGGTAACCATACAGTATACTGTGAAC |
| | | GTTGCCCAAAAAAGTTTGGAAATGGTATATAAAGGTAAATTGACTG |
| | | CTGGTGAAGCGACGCCAATAAATTTAACAAATCATAGTTATTTCAA |
| | | TCTGAACAAGCCATATGGAGACACTATTGAGGGTACGGAGATTATG |
| | | GTGCGTTCAAAAAAATCTGTTGATGTCGACAAAAACATGATTCCTA |
| | | CGGGTAATATCGTCGATAGAGAAATTGCTACCTTTAACTCTACAAA |
| | | GCCAACGGTCTTAGGCCCCAAAAATCCCCAGTTTGATTGTTGTTTTG |
| | | TGGTGGATGAAAATGCTAAGCCAAGTCAAATCAATACTCTAAACAA |
| | | TGAATTGACGCTTATTGTCAAGGCTTTTCATCCCGATTCCAATATTA |
| | | CATTAGAAGTTTTAAGTACAGAGCCAACTTATCAATTTTATACCGGT |
| | | GATTTCTTGTCTGCTGGTTACGAAGCAAGACAAGGTTTTGCAATTGA |
| | | GCCTGGTAGATACATTGATGCTATCAATCAAGAGAACTGGAAAGAT |
| | | TGTGTAACCTTGAAAAACGGTGAAACTTACGGGTCCAAGATTGTCT |
| | | ACAGATTTTCCTGA |
| 75 | hGalT codon | GGTAGAGATTTGTCTAGATTGCCACAGTTGGTTGGTGTTTCCACTCC |
| | optimized (XB) | ATTGCAAGGAGGTTCTAACTCTGCTGCTGCTATTGGTCAATCTTCCG |
| | | GTGAGTTGAGAACTGGTGGAGCTAGACCACCTCCACCATTGGGAGC |
| | | TTCCTCTCAACCAAGACCAGGTGGTGATTCTTCTCCAGTTGTTGACT |
| | | CTGGTCCAGGTCCAGCTTCTAACTTGACTTCCGTTCCAGTTCCACAC |
| | | ACTACTGCTTTGTCCTTGCCAGCTTGTCCAGAAGAATCCCCATTGTT |
| | | GGTTGGTCCAATGTTGATCGAGTTCAACATGCCAGTTGACTTGGAGT |
| | | TGGTTGCTAAGCAGAACCCAAACGTTAAGATGGGTGGTAGATACGC |
| | | TCCAAGAGACTGTGTTTCCCCACACAAAGTTGCTATCATCATCCCAT |
| | | TCAGAAACAGACAGGAGCACTTGAAGTACTGGTTGTACTACTTGCA |
| | | CCCAGTTTTGCAAAGACAGCAGTTGGACTACGGTATCTACGTTATC |
| | | AACCAGGCTGGTGACACTATTTTCAACAGAGCTAAGTTGTTGAATG |
| | | TTGGTTTCCAGGAGGCTTTGAAGGATTACGACTACACTTGTTTCGTT |
| | | TTCTCCGACGTTGACTTGATTCCAATGAACGACCACAACGCTTACAG |
| | | ATGTTTCTCCCAGCCAAGACACATTTCTGTTGCTATGGACAAGTTCG |
| | | GTTTCTCCTTGCCATACGTTCAATACTTCGGTGGTGTTTCCGCTTTGT |
| | | CCAAGCAGCAGTTCTTGACTATCAACGGTTTCCCAAACAATTACTGG |
| | | GGATGGGGTGGTGAAGATGACGACATCTTTAACAGATTGGTTTTCA |
| | | GAGGAATGTCCATCTCTAGACCAAACGCTGTTGTTGGTAGATGTAG |
| | | AATGATCAGACACTCCAGAGACAAGAAGAACGAGCCAAACCCACA |
| | | AAGATTCGACAGAATCGCTCACACTAAGGAAACTATGTTGTCCGAC |
| | | GGATTGAACTCCTTGACTTACCAGGTTTTGGACGTTCAGAGATACCC |
| | | ATTGTACACTCAGATCACTGTTGACATCGGTACTCCATCCTAG |
| 76 | DNA encodes | ATGGCCCTCTTTCTCAGTAAGAGACTGTTGAGATTTACCGTCATTGC |
| | ScMnt1 (Kre2)(33) | AGGTGCGGTTATTGTTCTCCTCCTAACATTGAATTCCAACAGTAGAA |
| | | CTCAGCAATATATTCCGAGTTCCATCTCCGCTGCATTTGATTTTACCT |
| | | CAGGATCTATATCCCCTGAACAACAAGTCATCGGGCGCGCC |
| 77 | DNA encodes | ATGAATAGCATACACATGAACGCCAATACGCTGAAGTACATCAGCC |
| | DmUGT | TGCTGACGCTGACCCTGCAGAATGCCATCCTGGGCCTCAGCATGCG |
| | | CTACGCCCGCACCCGGCCAGGCGACATCTTCCTCAGCTCCACGGCC |
| | | GTACTCATGGCAGAGTTCGCCAAACTGATCACGTGCCTGTTCCTGGT |
| | | CTTCAACGAGGAGGGCAAGGATGCCCAGAAGTTTGTACGCTCGCTG |
| | | CACAAGACCATCATTGCGAATCCCATGGACACGCTGAAGGTGTGCG |
| | | TCCCCTCGCTGGTCTATATCGTTCAAAACAATCTGCTGTACGTCTCT |
| | | GCCTCCCATTTGGATGCGGCCACCTACCAGGTGACGTACCAGCTGA |
| | | AGATTCTCACCACGGCCATGTTCGCGGTTGTCATTCTGCGCCGCAAG |
| | | CTGCTGAACACGCAGTGGGGTGCGCTGCTGCTCCTGGTGATGGGCA |
| | | TCGTCCTGGTGCAGTTGGCCCAAACGGAGGGTCCGACGAGTGGCTC |
| | | AGCCGGTGGTGCCGCAGCTGCAGCCACGGCCGCCTCCTCTGGCGGT |
| | | GCTCCCGAGCAGAACAGGATGCTCGGACTGTGGGCCGCACTGGGCG |
| | | CCTGCTTCCTCTCCGGATTCGCGGGCATCTACTTTGAGAAGATCCTC |
| | | AAGGGTGCCGAGATCTCCGTGTGGATGCGGAATGTGCAGTTGAGTC |
| | | TGCTCAGCATTCCCTTCGGCCTGCTCACCTGTTTCGTTAACGACGGC |
| | | AGTAGGATCTTCGACCAGGGATTCTTCAAGGGCTACGATCTGTTTGT |
| | | CTGGTACCTGGTCCTGCTGCAGGCCGGCGGTGGATTGATCGTTGCCG |
| | | TGGTGGTCAAGTACGCGGATAACATTCTCAAGGGCTTCGCCACCTC |
| | | GCTGGCCATCATCATCTCGTGCGTGGCCTCCATATACATCTTCGACT |
| | | TCAATCTCACGCTGCAGTTCAGCTTCGGAGCTGGCCTGGTCATCGCC |
| | | TCCATATTTCTCTACGGCTACGATCCGGCCAGGTCGGCGCCGAAGCC |
| | | AACTATGCATGGTCCTGGCGGCGATGAGGAGAAGCTGCTGCCGCGC |
| | | GTCTAG |
| 78 | Sequence of the | TGGACACAGGAGACTCAGAAACAGACACAGAGCGTTCTGAGTCCTG |
| | PpOCH1 promoter: | GTGCTCCTGACGTAGGCCTAGAACAGGAATTATTGGCTTTATTTGTT |
| | | TGTCCATTTCATAGGCTTGGGGTAATAGATAGATGACAGAGAAATA |
| | | GAGAAGACCTAATATTTTTTGTTCATGGCAAATCGCGGGTTCGCGGT |
| | | CGGGTCACACACGGAGAAGTAATGAGAAGAGCTGGTAATCTGGGG |
| | | TAAAAGGGTTCAAAAGAAGGTCGCCTGGTAGGGATGCAATACAAG |
| | | GTTGTCTTGGAGTTTACATTGACCAGATGATTTGGCTTTTTCTCTGTT |
| | | CAATTCACATTTTTCAGCGAGAATCGGATTGACGGAGAAATGGCGG |
| | | GGTGTGGGGTGGATAGATGGCAGAAATGCTCGCAATCACCGCGAA |
| | | AGAAAGACTTTATGGAATAGAACTACTGGGTGGTGTAAGGATTACA |
| | | TAGCTAGTCCAATGGAGTCCGTTGGAAAGGTAAGAAGAAGCTAAA |
| | | ACCGGCTAAGTAACTAGGGAAGAATGATCAGACTTTGATTTGATGA |
| | | GGTCTGAAAATACTCTGCTGCTTTTTCAGTTGCTTTTTCCCTGCAACC |
| | | TATCATTTTCCTTTTCATAAGCCTGCCTTTTCTGTTTTCACTTATATG |
| | | AGTTCCGCCGAGACTTCCCCAAATTCTCTCCTGGAACATTCTCTATC |
| | | GCTCTCCTTCCAAGTTGCGCCCCCTGGCACTGCCTAGTAATATTACC |
| | | ACGCGACTTATATTCAGTTCCACAATTTCCAGTGTTCGTAGCAAATA |
| | | TCATCAGCC |
| 79 | Sequence of the | AATATATACCTCATTTGTTCAATTTGGTGTAAAGAGTGTGGCGGATA |
| | PpALG12 | GACTTCTTGTAAATCAGGAAAGCTACAATTCCAATTGCTGCAAAAA |
| | terminator: | ATACCAATGCCCATAAACCAGTATGAGCGGTGCCTTCGACGGATTG |
| | | CTTACTTTCCGACCCTTTGTCGTTTGATTCTTCTGCCTTTGGTGAGTC |
| | | AGTTTGTTTCGACTTTATATCTGACTCATCAACTTCCTTTACGGTTGC |
| | | GTTTTTAATCATAATTTTAGCCGTTGGCTTATTATCCCTTGAGTTGGT |
| | | AGGAGTTTTGATGATGCTG |
| 80 | Sequence of the 5′- | TAACTGGCCCTTTGACGTTTCTGACAATAGTTCTAGAGGAGTCGTCC |
| | Region used for | AAAAACTCAACTCTGACTTGGGTGACACCACCACGGGATCCGGTTC |
| | knock out of | TTCCGAGGACCTTGATGACCTTGGCTAATGTAACTGGAGTTTTAGTA |
| | PpHIS1: | TCCATTTTAAGATGTGTGTTTCTGTAGGTTCTGGGTTGGAAAAAAAT |
| | | TTTAGACACCAGAAGAGAGGAGTGAACTGGTTTGCGTGGGTTTAGA |
| | | CTGTGTAAGGCACTACTCTGTCGAAGTTTTAGATAGGGGTTACCCGC |
| | | TCCGATGCATGGGAAGCGATTAGCCCGGCTGTTGCCCGTTTGGTTTT |
| | | TGAAGGGTAATTTTCAATATCTCTGTTTGAGTCATCAATTTCATATT |
| | | CAAAGATTCAAAAACAAAATCTGGTCCAAGGAGCGCATTTAGGATT |
| | | ATGGAGTTGGCGAATCACTTGAACGATAGACTATTATTTGC |
| 81 | Sequence of the 3′- | GTGACATTCTTGTCTTTGAGATCAGTAATTGTAGAGCATAGATAGA |
| | Region used for | ATAATATTCAAGACCAACGGCTTCTCTTCGGAAGCTCCAAGTAGCTT |
| | knock out of | ATAGTGATGAGTACCGGCATATATTTATAGGCTTAAAATTTCGAGG |
| | PpHIS1: | GTTCACTATATTCGTTTAGTGGGAAGAGTTCCTTTCACTCTTGTTATC |
| | | TATATTGTCAGCGTGGACTGTTTATAACTGTACCAACTTAGTTTCTTT |
| | | CAACTCCAGGTTAAGAGACATAAATGTCCTTTGATGCTGACAATAA |
| | | TCAGTGGAATTCAAGGAAGGACAATCCCGACCTCAATCTGTTCATT |
| | | AATGAAGAGTTCGAATCGTCCTTAAATCAAGCGCTAGACTCAATTG |
| | | TCAATGAGAACCCTTTCTTTGACCAAGAAACTATAAATAGATCGAA |
| | | TGACAAAGTTGGAAATGAGTCCATTAGCTTACATGATATTGAGCAG |
| | | GCAGACCAAAATAAACCGTCCTTTGAGAGCGATATTGATGGTTCGG |
| | | CGCCGTTGATAAGAGACGACAAATTGCCAAAGAAACAAAGCTGGG |
| | | GGCTGAGCAATTTTTTTTCAAGAAGAAATAGCATATGTTTACCACTA |
| | | CATGAAAATGATTCAAGTGTTGTTAAGACCGAAAGATCTATTGCAG |
| | | TGGGAACACCCCATCTTCAATACTGCTTCAATGGAATCTCCAATGCC |
| | | AAGTACAATGCATTTACCTTTTTCCCAGTCATCCTATACGAGCAATT |
| | | CAAATTTTTTTTCAATTTATACTTTACTTTAGTGGCTCTCTCTCAAGC |
| | | GATACCGCAACTTCGCATTGGATATCTTTCTTCGTATGTCGTCCCAC |
| | | TTTTGTTTGTACTCATAGTGACCATGTCAAAAGAGGCGATGGATGAT |
| | | ATTCAACGCCGAAGAAGGGATAGAGAACAGAACAATGAACCATAT |
| | | GAGGTTCTGTCCAGCCCATCACCAGTTTTGTCCAAAAACTTAAAATG |
| | | TGGTCACTTGGTTCGATTGCATAAGGGAATGAGAGTGCCCGCAGAT |
| | | ATGGTTCTTGTCCAGTCAAGCGAATCCACCGGAGAGTCATTTATCA |
| | | AGACAGATCAGCTGGATGGTGAGACTGATTGGAAGCTTCGGATTGT |
| | | TTCTCCAGTTACACAATCGTTACCAATGACTGAACTTCAAAATGTCG |
| | | CCATCACTGCAAGCGCACCCTCAAAATCAATTCACTCCTTTCTTGGA |
| | | AGATTGACCTACAATGGGCAATCATATGGTCTTACGATAGACAACA |
| | | CAATGTGGTGTAATACTGTATTAGCTTCTGGTTCAGCAATTGGTTGT |
| | | ATAATTTACACAGGTAAAGATACTCGACAATCGATGAACACAACTC |
| | | AGCCCAAACTGAAAACGGGCTTGTTAGAACTGGAAATCAATAGTTT |
| | | GTCCAAGATCTTATGTGTTTGTGTGTTTGCATTATCTGTCATCTTAGT |
| | | GCTATTCCAAGGAATAGCTGATGATTGGTACGTCGATATCATGCGG |
| | | TTTCTCATTCTATTCTCCACTATTATCCCAGTGTCTCTGAGAGTTAAC |
| | | CTTGATCTTGGAAAGTCAGTCCATGCTCATCAAATAGAAACTGATA |
| | | GCTCAATACCTGAAACCGTTGTTAGAACTAGTACAATACCGGAAGA |
| | | CCTGGGAAGAATTGAATACCTATTAAGTGACAAAACTGGAACTCTT |
| | | ACTCAAAATGATATGGAAATGAAAAAACTACACCTAGGAACAGTCT |
| | | CTTATGCTGGTGATACCATGGATATTATTTCTGATCATGTTAAAGGT |
| | | CTTAATAACGCTAAAACATCGAGGAAAGATCTTGGTATGAGAATAA |
| | | GAGATTTGGTTACAACTCTGGCCATCTG |
| 82 | DNA encodes | AGAGACGATCCAATTAGACCTCCATTGAAGGTTGCTAGATCCCCAA |
| | Drosophila | GACCAGGTCAATGTCAAGATGTTGTTCAGGACGTCCCAAACGTTGA |
| | melanogaster ManII | TGTCCAGATGTTGGAGTTGTACGATAGAATGTCCTTCAAGGACATT |
| | codon-optimized | GATGGTGGTGTTTGGAAGCAGGGTTGGAACATTAAGTACGATCCAT |
| | (KD) | TGAAGTACAACGCTCATCACAAGTTGAAGGTCTTCGTTGTCCCACAC |
| | | TCCCACAACGATCCTGGTTGGATTCAGACCTTCGAGGAATACTACC |
| | | AGCACGACACCAAGCACATCTTGTCCAACGCTTTGAGACATTTGCA |
| | | CGACAACCCAGAGATGAAGTTCATCTGGGCTGAAATCTCCTACTTC |
| | | GCTAGATTCTACCACGATTTGGGTGAGAACAAGAAGTTGCAGATGA |
| | | AGTCCATCGTCAAGAACGGTCAGTTGGAATTCGTCACTGGTGGATG |
| | | GGTCATGCCAGACGAGGCTAACTCCCACTGGAGAAACGTTTTGTTG |
| | | CAGTTGACCGAAGGTCAAACTTGGTTGAAGCAATTCATGAACGTCA |
| | | CTCCAACTGCTTCCTGGGCTATCGATCCATTCGGACACTCTCCAACT |
| | | ATGCCATACATTTTGCAGAAGTCTGGTTTCAAGAATATGTTGATCCA |
| | | GAGAACCCACTACTCCGTTAAGAAGGAGTTGGCTCAACAGAGACAG |
| | | TTGGAGTTCTTGTGGAGACAGATCTGGGACAACAAAGGTGACACTG |
| | | CTTTGTTCACCCACATGATGCCATTCTACTCTTACGACATTCCTCATA |
| | | CCTGTGGTCCAGATCCAAAGGTTTGTTGTCAGTTCGATTTCAAAAGA |
| | | ATGGGTTCCTTCGGTTTGTCTTGTCCATGGAAGGTTCCACCTAGAAC |
| | | TATCTCTGATCAAAATGTTGCTGCTAGATCCGATTTGTTGGTTGATC |
| | | AGTGGAAGAAGAAGGCTGAGTTGTACAGAACCAACGTCTTGTTGAT |
| | | TCCATTGGGTGACGACTTCAGATTCAAGCAGAACACCGAGTGGGAT |
| | | GTTCAGAGAGTCAACTACGAAAGATTGTTCGAACACATCAACTCTC |
| | | AGGCTCACTTCAATGTCCAGGCTCAGTTCGGTACTTTGCAGGAATAC |
| | | TTCGATGCTGTTCACCAGGCTGAAAGAGCTGGACAAGCTGAGTTCC |
| | | CAACCTTGTCTGGTGACTTCTTCACTTACGCTGATAGATCTGATAAC |
| | | TACTGGTCTGGTTACTACACTTCCAGACCATACCATAAGAGAATGG |
| | | ACAGAGTCTTGATGCACTACGTTAGAGCTGCTGAAATGTTGTCCGCT |
| | | TGGCACTCCTGGGACGGTATGGCTAGAATCGAGGAAAGATTGGAGC |
| | | AGGCTAGAAGAGAGTTGTCCTTGTTCCAGCACCACGACGGTATTAC |
| | | TGGTACTGCTAAAACTCACGTTGTCGTCGACTACGAGCAAAGAATG |
| | | CAGGAAGCTTTGAAAGCTTGTCAAATGGTCATGCAACAGTCTGTCT |
| | | ACAGATTGTTGACTAAGCCATCCATCTACTCTCCAGACTTCTCCTTC |
| | | TCCTACTTCACTTTGGACGACTCCAGATGGCCAGGTTCTGGTGTTGA |
| | | GGACTCTAGAACTACCATCATCTTGGGTGAGGATATCTTGCCATCCA |
| | | AGCATGTTGTCATGCACAACACCTTGCCACACTGGAGAGAGCAGTT |
| | | GGTTGACTTCTACGTCTCCTCTCCATTCGTTTCTGTTACCGACTTGGC |
| | | TAACAATCCAGTTGAGGCTCAGGTTTCTCCAGTTTGGTCTTGGCACC |
| | | ACGACACTTTGACTAAGACTATCCACCCACAAGGTTCCACCACCAA |
| | | GTACAGAATCATCTTCAAGGCTAGAGTTCCACCAATGGGTTTGGCT |
| | | ACCTACGTTTTGACCATCTCCGATTCCAAGCCAGAGCACACCTCCTA |
| | | CGCTTCCAATTTGTTGCTTAGAAAGAACCCAACTTCCTTGCCATTGG |
| | | GTCAATACCCAGAGGATGTCAAGTTCGGTGATCCAAGAGAGATCTC |
| | | CTTGAGAGTTGGTAACGGTCCAACCTTGGCTTTCTCTGAGCAGGGTT |
| | | TGTTGAAGTCCATTCAGTTGACTCAGGATTCTCCACATGTTCCAGTT |
| | | CACTTCAAGTTCTTGAAGTACGGTGTTAGATCTCATGGTGATAGATC |
| | | TGGTGCTTACTTGTTCTTGCCAAATGGTCCAGCTTCTCCAGTCGAGT |
| | | TGGGTCAGCCAGTTGTCTTGGTCACTAAGGGTAAATTGGAGTCTTCC |
| | | GTTTCTGTTGGTTTGCCATCTGTCGTTCACCAGACCATCATGAGAGG |
| | | TGGTGCTCCAGAGATTAGAAATTTGGTCGATATTGGTTCTTTGGACA |
| | | ACACTGAGATCGTCATGAGATTGGAGACTCATATCGACTCTGGTGA |
| | | TATCTTCTACACTGATTTGAATGGATTGCAATTCATCAAGAGGAGA |
| | | AGATTGGACAAGTTGCCATTGCAGGCTAACTACTACCCAATTCCATC |
| | | TGGTATGTTCATTGAGGATGCTAATACCAGATTGACTTTGTTGACCG |
| | | GTCAACCATTGGGTGGATCTTCTTTGGCTTCTGGTGAGTTGGAGATT |
| | | ATGCAAGATAGAAGATTGGCTTCTGATGATGAAAGAGGTTTGGGTC |
| | | AGGGTGTTTTGGACAACAAGCCAGTTTTGCATATTTACAGATTGGTC |
| | | TTGGAGAAGGTTAACAACTGTGTCAGACCATCTAAGTTGCATCCAG |
| | | CTGGTTACTTGACTTCTGCTGCTCACAAAGCTTCTCAGTCTTTGTTGG |
| | | ATCCATTGGACAAGTTCATCTTCGCTGAAAATGAGTGGATCGGTGC |
| | | TCAGGGTCAATTCGGTGGTGATCATCCATCTGCTAGAGAGGATTTG |
| | | GATGTCTCTGTCATGAGAAGATTGACCAAGTCTTCTGCTAAAACCC |
| | | AGAGAGTTGGTTACGTTTTGCACAGAACCAATTTGATGCAATGTGG |
| | | TACTCCAGAGGAGCATACTCAGAAGTTGGATGTCTGTCACTTGTTGC |
| | | CAAATGTTGCTAGATGTGAGAGAACTACCTTGACTTTCTTGCAGAAT |
| | | TTGGAGCACTTGGATGGTATGGTTGCTCCAGAAGTTTGTCCAATGG |
| | | AAACCGCTGCTTACGTCTCTTCTCACTCTTCTTGA |
| 83 | DNA encodes Mnn2 | ATGCTGCTTACCAAAAGGTTTTCAAAGCTGTTCAAGCTGACGTTCAT |
| | leader (53) | AGTTTTGATATTGTGCGGGCTGTTCGTCATTACAAACAAATACATGG |
| | | ATGAGAACACGTCG |
| 84 | Sequence of the | CAAGTTGCGTCCGGTATACGTAACGTCTCACGATGATCAAAGATAA |
| | PpHIS1 auxotrophic | TACTTAATCTTCATGGTCTACTGAATAACTCATTTAAACAATTGACT |
| | marker: | AATTGTACATTATATTGAACTTATGCATCCTATTAACGTAATCTTCT |
| | | GGCTTCTCTCTCAGACTCCATCAGACACAGAATATCGTTCTCTCTAA |
| | | CTGGTCCTTTGACGTTTCTGACAATAGTTCTAGAGGAGTCGTCCAAA |
| | | AACTCAACTCTGACTTGGGTGACACCACCACGGGATCCGGTTCTTCC |
| | | GAGGACCTTGATGACCTTGGCTAATGTAACTGGAGTTTTAGTATCCA |
| | | TTTTAAGATGTGTGTTTCTGTAGGTTCTGGGTTGGAAAAAAATTTTA |
| | | GACACCAGAAGAGAGGAGTGAACTGGTTTGCGTGGGTTTAGACTGT |
| | | GTAAGGCACTACTCTGTCGAAGTTTTAGATAGGGGTTACCCGCTCC |
| | | GATGCATGGGAAGCGATTAGCCCGGCTGTTGCCCGTTTGGTTTTTGA |
| | | AGGGTAATTTTCAATATCTCTGTTTGAGTCATCAATTTCATATTCAA |
| | | AGATTCAAAAACAAAATCTGGTCCAAGGAGCGCATTTAGGATTATG |
| | | GAGTTGGCGAATCACTTGAACGATAGACTATTATTTGCTGTTCCTAA |
| | | AGAGGGCAGATTGTATGAGAAATGCGTTGAATTACTTAGGGGATCA |
| | | GATATTCAGTTTCGAAGATCCAGTAGATTGGATATAGCTTTGTGCAC |
| | | TAACCTGCCCCTGGCATTGGTTTTCCTTCCAGCTGCTGACATTCCCA |
| | | CGTTTGTAGGAGAGGGTAAATGTGATTTGGGTATAACTGGTATTGA |
| | | CCAGGTTCAGGAAAGTGACGTAGATGTCATACCTTTATTAGACTTG |
| | | AATTTCGGTAAGTGCAAGTTGCAGATTCAAGTTCCCGAGAATGGTG |
| | | ACTTGAAAGAACCTAAACAGCTAATTGGTAAAGAAATTGTTTCCTC |
| | | CTTTACTAGCTTAACCACCAGGTACTTTGAACAACTGGAAGGAGTT |
| | | AAGCCTGGTGAGCCACTAAAGACAAAAATCAAATATGTTGGAGGGT |
| | | CTGTTGAGGCCTCTTGTGCCCTAGGAGTTGCCGATGCTATTGTGGAT |
| | | CTTGTTGAGAGTGGAGAAACCATGAAAGCGGCAGGGCTGATCGATA |
| | | TTGAAACTGTTCTTTCTACTTCCGCTTACCTGATCTCTTCGAAGCATC |
| | | CTCAACACCCAGAACTGATGGATACTATCAAGGAGAGAATTGAAGG |
| | | TGTACTGACTGCTCAGAAGTATGTCTTGTGTAATTACAACGCACCTA |
| | | GAGGTAACCTTCCTCAGCTGCTAAAACTGACTCCAGGCAAGAGAGC |
| | | TGCTACCGTTTCTCCATTAGATGAAGAAGATTGGGTGGGAGTGTCCT |
| | | CGATGGTAGAGAAGAAAGATGTTGGAAGAATCATGGACGAATTAA |
| | | AGAAACAAGGTGCCAGTGACATTCTTGTCTTTGAGATCAGTAATTG |
| | | TAGAGCATAGATAGAATAATATTCAAGACCAACGGCTTCTCTTCGG |
| | | AAGCTCCAAGTAGCTTATAGTGATGAGTACCGGCATATATTTATAG |
| | | GCTTAAAATTTCGAGGGTTCACTATATTCGTTTAGTGGGAAGAGTTC |
| | | CTTTCACTCTTGTTATCTATATTGTCAGCGTGGACTGTTTATAACTGT |
| | | ACCAACTTAGTTTCTTTCAACTCCAGGTTAAGAGACATAAATGTCCT |
| | | TTGATGC |
| 85 | DNA encodes Rat | TCCTTGGTTTACCAATTGAACTTCGACCAGATGTTGAGAAACGTTGA |
| | GnT II | CAAGGACGGTACTTGGTCTCCTGGTGAGTTGGTTTTGGTTGTTCAGG |
| | (TC) | TTCACAACAGACCAGAGTACTTGAGATTGTTGATCGACTCCTTGAG |
| | Codon-optimized | AAAGGCTCAAGGTATCAGAGAGGTTTTGGTTATCTTCTCCCACGATT |
| | | TCTGGTCTGCTGAGATCAACTCCTTGATCTCCTCCGTTGACTTCTGTC |
| | | CAGTTTTGCAGGTTTTCTTCCCATTCTCCATCCAATTGTACCCATCTG |
| | | AGTTCCCAGGTTCTGATCCAAGAGACTGTCCAAGAGACTTGAAGAA |
| | | GAACGCTGCTTTGAAGTTGGGTTGTATCAACGCTGAATACCCAGAT |
| | | TCTTTCGGTCACTACAGAGAGGCTAAGTTCTCCCAAACTAAGCATCA |
| | | TTGGTGGTGGAAGTTGCACTTTGTTTGGGAGAGAGTTAAGGTTTTGC |
| | | AGGACTACACTGGATTGATCTTGTTCTTGGAGGAGGATCATTACTTG |
| | | GCTCCAGACTTCTACCACGTTTTCAAGAAGATGTGGAAGTTGAAGC |
| | | AACAAGAGTGTCCAGGTTGTGACGTTTTGTCCTTGGGAACTTACACT |
| | | ACTATCAGATCCTTCTACGGTATCGCTGACAAGGTTGACGTTAAGA |
| | | CTTGGAAGTCCACTGAACACAACATGGGATTGGCTTTGACTAGAGA |
| | | TGCTTACCAGAAGTTGATCGAGTGTACTGACACTTTCTGTACTTACG |
| | | ACGACTACAACTGGGACTGGACTTTGCAGTACTTGACTTTGGCTTGT |
| | | TTGCCAAAAGTTTGGAAGGTTTTGGTTCCACAGGCTCCAAGAATTTT |
| | | CCACGCTGGTGACTGTGGAATGCACCACAAGAAAACTTGTAGACCA |
| | | TCCACTCAGTCCGCTCAAATTGAGTCCTTGTTGAACAACAACAAGC |
| | | AGTACTTGTTCCCAGAGACTTTGGTTATCGGAGAGAAGTTTCCAATG |
| | | GCTGCTATTTCCCCACCAAGAAAGAATGGTGGATGGGGTGATATTA |
| | | GAGACCACGAGTTGTGTAAATCCTACAGAAGATTGCAGTAG |
| 86 | DNA encodes Mnn2 | ATGCTGCTTACCAAAAGGTTTTCAAAGCTGTTCAAGCTGACGTTCAT |
| | leader (54) | AGTTTTGATATTGTGCGGGCTGTTCGTCATTACAAACAAATACATGG |
| | The last 9 | ATGAGAACACGTCGGTCAAGGAGTACAAGGAGTACTTAGACAGAT |
| | nucleotides are the | ATGTCCAGAGTTACTCCAATAAGTATTCATCTTCCTCAGACGCCGCC |
| | linker containing the | AGCGCTGACGATTCAACCCCATTGAGGGACAATGATGAGGCAGGCA |
| | AscI restriction site) | ATGAAAAGTTGAAAAGCTTCTACAACAACGTTTTCAACTTTCTAATG |
| | | GTTGATTCGCCCGGGCGCGCC |
| 87 | Sequence of the 5′- | GATCTGGCCTTCCCTGAATTTTTACGTCCAGCTATACGATCCGTTGT |
| | Region used for | GACTGTATTTCCTGAAATGAAGTTTCAACCTAAAGTTTTGGTTGTAC |
| | knock out of | TTGCTCCACCTACCACGGAAACTAATATCGAAACCAATGAAAAAGT |
| | PpARG1: | AGAACTGGAATCGTCAATCGAAATTCGCAACCAAGTGGAACCCAAA |
| | | GACTTGAATCTTTCTAAAGTCTATTCTAGTGACACTAATGGCAACAG |
| | | AAGATTTGAGCTGACTTTTCAAATGAATCTCAATAATGCAATATCA |
| | | ACATCAGACAATCAATGGGCTTTGTCTAGTGACACAGGATCAATTA |
| | | TAGTAGTGTCTTCTGCAGGAAGAATAACTTCCCCGATCCTAGAAGT |
| | | CGGGGCATCCGTCTGTGTCTTAAGATCGTACAACGAACACCTTTTGG |
| | | CAATAACTTGTGAAGGAACATGCTTTTCATGGAATTTAAAGAAGCA |
| | | AGAATGTGTTCTAAACAGCATTTCATTAGCACCTATAGTCAATTCAC |
| | | ACATGCTAGTTAAGAAAGTTGGAGATGCAAGGAACTATTCTATTGT |
| | | ATCTGCCGAAGGAGACAACAATCCGTTACCCCAGATTCTAGACTGC |
| | | GAACTTTCCAAAAATGGCGCTCCAATTGTGGCTCTTAGCACGAAAG |
| | | ACATCTACTCTTATTCAAAGAAAATGAAATGCTGGATCCATTTGATT |
| | | GATTCGAAATACTTTGAATTGTTGGGTGCTGACAATGCACTGTTTGA |
| | | GTGTGTGGAAGCGCTAGAAGGTCCAATTGGAATGCTAATTCATAGA |
| | | TTGGTAGATGAGTTCTTCCATGAAAACACTGCCGGTAAAAAACTCA |
| | | AACTTTACAACAAGCGAGTACTGGAGGACCTTTCAAATTCACTTGA |
| | | AGAACTAGGTGAAAATGCGTCTCAATTAAGAGAGAAACTTGACAA |
| | | ACTCTATGGTGATGAGGTTGAGGCTTCTTGACCTCTTCTCTCTATCT |
| | | GCGTTTCTTTTTTTTTTTTTTTTTTTTTTTTTTTCAGTTGAGCCAGACC |
| | | GCGCTAAACGCATACCAATTGCCAAATCAGGCAATTGTGAGACAGT |
| | | GGTAAAAAAGATGCCTGCAAAGTTAGATTCACACAGTAAGAGAGA |
| | | TCCTACTCATAAATGAGGCGCTTATTTAGTAGCTAGTGATAGCCACT |
| | | GCGGTTCTGCTTTATGCTATTTGTTGTATGCCTTACTATCTTTGTTTG |
| | | GCTCCTTTTTCTTGACGTTTTCCGTTGGAGGGACTCCCTATTCTGAGT |
| | | CATGAGCCGCACAGATTATCGCCCAAAATTGACAAAATCTTCTGGC |
| | | GAAAAAAGTATAAAAGGAGAAAAAAGCTCACCCTTTTCCAGCGTA |
| | | GAAAGTATATATCAGTCATTGAAGAC |
| 88 | Sequence of the 3′- | GGGACTTTAACTCAAGTAAAAGGATAGTTGTACAATTATATATACG |
| | Region used for | AAGAATAAATCATTACAAAAAGTATTCGTTTCTTTGATTCTTAACAG |
| | knock out of | GATTCATTTTCTGGGTGTCATCAGGTACAGCGCTGAATATCTTGAAG |
| | PpARG1: | TTAACATCGAGCTCATCATCGACGTTCATCACACTAGCCACGTTTCC |
| | | GCAACGGTAGCAATAATTAGGAGCGGACCACACAGTGACGACATCT |
| | | TTCTCTTTGAAATGGTATCTGAAGCCTTCCATGACCAATTGATGGGC |
| | | TCTAGCGATGAGTTGCAAGTTATTAATGTGGTTGAACTCACGTGCTA |
| | | CTCGAGCACCGAATAACCAGCCAGCTCCACGAGGAGAAACAGCCC |
| | | AACTGTCGACTTCATCTGGGTCAGACCAAACCAAGTCACAAAATCC |
| | | TCCTTCATGAGGGACCTCTTGCGCTCGGCTGAGAACTCTGATTTGAT |
| | | CTAACATGCGAATATCGGGAGAGAGACCACCATGGATACATAATAT |
| | | TTTACCATCAATGATGGCACTAAGGGTTAAAAAGTCGAACACCTGG |
| | | CAACAGTACTTCCAGACAGTGGTGGAACCATATTTATTGAGACATT |
| | | CCTCATAAAATCCATAAACCTGAGTGATCTGTCTGGATTCATGATTT |
| | | CCCCTTACCAATGTGATATGTTGAGGAAACTTAATTTTTAAAATCAT |
| | | GAGTAACGTGAACGTCTCCAACGAGAAATAGCCTCTATCCACATAG |
| | | TCTCCTAGGAAGATATAGTTCTGTTTTATTCCATTAGAGGAGGATCC |
| | | GGGAAACCCACCACTAATCTTGAAAAGTTCCAGTAGATCGTGAAAT |
| | | TGGCCGTGAATATCTCCGCATACTGTCACTGGACTCTGCACTGGCTG |
| | | TATATTGGATTCCTCCATCAGCAAATCCTTCACCCGTTCGCAAAGAT |
| | | GCTTCATATCATTTTCACTTAAAGCCTTGCAGCTTTTGACTTCTTCAA |
| | | ACCACTGATCTGGTCCTCTTTCTGGCATGATTAAGGTCTATAATATT |
| | | TCTGAGCTGAGATGTAAAAAAAAATAATAAAAATGGGGAGTGAAA |
| | | AAGTGTGTAGCTTTTAGGAGTTTGGGATTGATACCCCAAAATGATCT |
| | | TTATGAGAATTAAAAGGTAGATACGCTTTTAATAAGAACACCTATC |
| | | TATAGTACTTTGTGGTCTTGAGTAATTGAGATGTTCAGCTTCTGAGG |
| | | TTTGCCGTTATTCTGGGATAGTAGTGCGCGACCAAACAACCCGCCA |
| | | GGCAAAGTGTGTTGTGCTCGAAGACGATTGCCAGAAGAGTAAGTCC |
| | | GTCCTGCCTCAGATGTTACACACTTTCTTCCCTAGACAGTCGATGCA |
| | | TCATCGGATTTAAACCTGAAACTTTGATGCCATGATACGCCTAGTCA |
| | | CGTCGACTGAGATTTTAGATAAGCCCCGATCCCTTTAGTACATTCCT |
| | | GTTATCCATGGATGGAATGGCCTGATA |
| 89 | Sequence of the 5′- | AAGCTTGTTCACCGTTGGGACTTTTCCGTGGACAATGTTGACTACTC |
| | Region used for | CAGGAGGGATTCCAGCTTTCTCTACTAGCTCAGCAATAATCAATGC |
| | knock out of BMT4 | AGCCCCAGGCGCCCGTTCTGATGGCTTGATGACCGTTGTATTGCCTG |
| | | TCACTATAGCCAGGGGTAGGGTCCATAAAGGAATCATAGCAGGGA |
| | | AATTAAAAGGGCATATTGATGCAATCACTCCCAATGGCTCTCTTGCC |
| | | ATTGAAGTCTCCATATCAGCACTAACTTCCAAGAAGGACCCCTTCA |
| | | AGTCTGACGTGATAGAGCACGCTTGCTCTGCCACCTGTAGTCCTCTC |
| | | AAAACGTCACCTTGTGCATCAGCAAAGACTTTACCTTGCTCCAATAC |
| | | TATGACGGAGGCAATTCTGTCAAAATTCTCTCTCAGCAATTCAACCA |
| | | ACTTGAAAGCAAATTGCTGTCTCTTGATGATGGAGACTTTTTTCCAA |
| | | GATTGAAATGCAATGTGGGACGACTCAATTGCTTCTTCCAGCTCCTC |
| | | TTCGGTTGATTGAGGAACTTTTGAAACCACAAAATTGGTCGTTGGGT |
| | | CATGTACATCAAACCATTCTGTAGATTTAGATTCGACGAAAGCGTT |
| | | GTTGATGAAGGAAAAGGTTGGATACGGTTTGTCGGTCTCTTTGGTAT |
| | | GGCCGGTGGGGTATGCAATTGCAGTAGAAGATAATTGGACAGCCAT |
| | | TGTTGAAGGTAGAGAAAAGGTCAGGGAACTTGGGGGTTATTTATAC |
| | | CATTTTACCCCACAAATAACAACTGAAAAGTACCCATTCCATAGTG |
| | | AGAGGTAACCGACGGAAAAAGACGGGCCCATGTTCTGGGACCAAT |
| | | AGAACTGTGTAATCCATTGGGACTAATCAACAGACGATTGGCAATA |
| | | TAATGAAATAGTTCGTTGAAAAGCCACGTCAGCTGTCTTTTCATTAA |
| | | CTTTGGTCGGACACAACATTTTCTACTGTTGTATCTGTCCTACTTTGC |
| | | TTATCATCTGCCACAGGGCAAGTGGATTTCCTTCTCGCGCGGCTGGG |
| | | TGAAAACGGTTAACGTGAA |
| 90 | Sequence of the 3′- | GCCTTGGGGGACTTCAAGTCTTTGCTAGAAACTAGATGAGGTCAGG |
| | Region used for | CCCTCTTATGGTTGTGTCCCAATTGGGCAATTTCACTCACCTAAAAA |
| | knock out of BMT4 | GCATGACAATTATTTAGCGAAATAGGTAGTATATTTTCCCTCATCTC |
| | | CCAAGCAGTTTCGTTTTTGCATCCATATCTCTCAAATGAGCAGCTAC |
| | | GACTCATTAGAACCAGAGTCAAGTAGGGGTGAGCTCAGTCATCAGC |
| | | CTTCGTTTCTAAAACGATTGAGTTCTTTTGTTGCTACAGGAAGCGCC |
| | | CTAGGGAACTTTCGCACTTTGGAAATAGATTTTGATGACCAAGAGC |
| | | GGGAGTTGATATTAGAGAGGCTGTCCAAAGTACATGGGATCAGGCC |
| | | GGCCAAATTGATTGGTGTGACTAAACCATTGTGTACTTGGACACTCT |
| | | ATTACAAAAGCGAAGATGATTTGAAGTATTACAAGTCCCGAAGTGT |
| | | TAGAGGATTCTATCGAGCCCAGAATGAAATCATCAACCGTTATCAG |
| | | CAGATTGATAAACTCTTGGAAAGCGGTATCCCATTTTCATTATTGAA |
| | | GAACTACGATAATGAAGATGTGAGAGACGGCGACCCTCTGAACGTA |
| | | GACGAAGAAACAAATCTACTTTTGGGGTACAATAGAGAAAGTGAAT |
| | | CAAGGGAGGTATTTGTGGCCATAATACTCAACTCTATCATTAATG |
| 91 | Sequence of the 5′- | CATATGGTGAGAGCCGTTCTGCACAACTAGATGTTTTCGAGCTTCGC |
| | Region used for | ATTGTTTCCTGCAGCTCGACTATTGAATTAAGATTTCCGGATATCTC |
| | knock out of BMT1 | CAATCTCACAAAAACTTATGTTGACCACGTGCTTTCCTGAGGCGAG |
| | | GTGTTTTATATGCAAGCTGCCAAAAATGGAAAACGAATGGCCATTT |
| | | TTCGCCCAGGCAAATTATTCGATTACTGCTGTCATAAAGACAGTGTT |
| | | GCAAGGCTCACATTTTTTTTTAGGATCCGAGATAAAGTGAATACAG |
| | | GACAGCTTATCTCTATATCTTGTACCATTCGTGAATCTTAAGAGTTC |
| | | GGTTAGGGGGACTCTAGTTGAGGGTTGGCACTCACGTATGGCTGGG |
| | | CGCAGAAATAAAATTCAGGCGCAGCAGCACTTATCGATG |
| 92 | Sequence of the 3′- | GAATTCACAGTTATAAATAAAAACAAAAACTCAAAAAGTTTGGGCT |
| | Region used for | CCACAAAATAACTTAATTTAAATTTTTGTCTAATAAATGAATGTAAT |
| | knock out of BMT1 | TCCAAGATTATGTGATGCAAGCACAGTATGCTTCAGCCCTATGCAG |
| | | CTACTAATGTCAATCTCGCCTGCGAGCGGGCCTAGATTTTCACTACA |
| | | AATTTCAAAACTACGCGGATTTATTGTCTCAGAGAGCAATTTGGCAT |
| | | TTCTGAGCGTAGCAGGAGGCTTCATAAGATTGTATAGGACCGTACC |
| | | AACAAATTGCCGAGGCACAACACGGTATGCTGTGCACTTATGTGGC |
| | | TACTTCCCTACAACGGAATGAAACCTTCCTCTTTCCGCTTAAACGAG |
| | | AAAGTGTGTCGCAATTGAATGCAGGTGCCTGTGCGCCTTGGTGTATT |
| | | GTTTTTGAGGGCCCAATTTATCAGGCGCCTTTTTTCTTGGTTGTTTTC |
| | | CCTTAGCCTCAAGCAAGGTTGGTCTATTTCATCTCCGCTTCTATACC |
| | | GTGCCTGATACTGTTGGATGAGAACACGACTCAACTTCCTGCTGCTC |
| | | TGTATTGCCAGTGTTTTGTCTGTGATTTGGATCGGAGTCCTCCTTACT |
| | | TGGAATGATAATAATCTTGGCGGAATCTCCCTAAACGGAGGCAAGG |
| | | ATTCTGCCTATGATGATCTGCTATCATTGGGAAGCTT |
| 93 | Sequence of the 5′- | GATATCTCCCTGGGGACAATATGTGTTGCAACTGTTCGTTGTTGGTG |
| | Region used for | CCCCAGTCCCCCAACCGGTACTAATCGGTCTATGTTCCCGTAACTCA |
| | knock out of BMT3 | TATTCGGTTAGAACTAGAACAATAAGTGCATCATTGTTCAACATTGT |
| | | GGTTCAATTGTCGAACATTGCTGGTGCTTATATCTACAGGGAAGAC |
| | | GATAAGCCTTTGTACAAGAGAGGTAACAGACAGTTAATTGGTATTT |
| | | CTTTGGGAGTCGTTGCCCTCTACGTTGTCTCCAAGACATACTACATT |
| | | CTGAGAAACAGATGGAAGACTCAAAAATGGGAGAAGCTTAGTGAA |
| | | GAAGAGAAAGTTGCCTACTTGGACAGAGCTGAGAAGGAGAACCTG |
| | | GGTTCTAAGAGGCTGGACTTTTTGTTCGAGAGTTAAACTGCATAATT |
| | | TTTTCTAAGTAAATTTCATAGTTATGAAATTTCTGCAGCTTAGTGTTT |
| | | ACTGCATCGTTTACTGCATCACCCTGTAAATAATGTGAGCTTTTTTC |
| | | CTTCCATTGCTTGGTATCTTCCTTGCTGCTGTTT |
| 94 | Sequence of the 3′- | ACAAAACAGTCATGTACAGAACTAACGCCTTTAAGATGCAGACCAC |
| | Region used for | TGAAAAGAATTGGGTCCCATTTTTCTTGAAAGACGACCAGGAATCT |
| | knock out of BMT3 | GTCCATTTTGTTTACTCGTTCAATCCTCTGAGAGTACTCAACTGCAG |
| | | TCTTGATAACGGTGCATGTGATGTTCTATTTGAGTTACCACATGATT |
| | | TTGGCATGTCTTCCGAGCTACGTGGTGCCACTCCTATGCTCAATCTT |
| | | CCTCAGGCAATCCCGATGGCAGACGACAAAGAAATTTGGGTTTCAT |
| | | TCCCAAGAACGAGAATATCAGATTGCGGGTGTTCTGAAACAATGTA |
| | | CAGGCCAATGTTAATGCTTTTTGTTAGAGAAGGAACAAACTTTTTTG |
| | | CTGAGC |
| 95 | Mouse CMP-sialic | ATGGCTCCAGCTAGAGAAAACGTTTCCTTGTTCTTCAAGTTGTACTG |
| | acid transporter | TTTGGCTGTTATGACTTTGGTTGCTGCTGCTTACACTGTTGCTTTGAG |
| | (MmCST) | ATACACTAGAACTACTGCTGAGGAGTTGTACTTCTCCACTACTGCTG |
| | Codon optimized | TTTGTATCACTGAGGTTATCAAGTTGTTGATCTCCGTTGGTTTGTTG |
| | | GCTAAGGAGACTGGTTCTTTGGGAAGATTCAAGGCTTCCTTGTCCG |
| | | AAAACGTTTTGGGTTCCCCAAAGGAGTTGGCTAAGTTGTCTGTTCCA |
| | | TCCTTGGTTTACGCTGTTCAGAACAACATGGCTTTCTTGGCTTTGTCT |
| | | AACTTGGACGCTGCTGTTTACCAAGTTACTTACCAGTTGAAGATCCC |
| | | ATGTACTGCTTTGTGTACTGTTTTGATGTTGAACAGAACATTGTCCA |
| | | AGTTGCAGTGGATCTCCGTTTTCATGTTGTGTGGTGGTGTTACTTTG |
| | | GTTCAGTGGAAGCCAGCTCAAGCTTCCAAAGTTGTTGTTGCTCAGA |
| | | ACCCATTGTTGGGTTTCGGTGCTATTGCTATCGCTGTTTTGTGTTCCG |
| | | GTTTCGCTGGTGTTTACTTCGAGAAGGTTTTGAAGTCCTCCGACACT |
| | | TCTTTGTGGGTTAGAAACATCCAGATGTACTTGTCCGGTATCGTTGT |
| | | TACTTTGGCTGGTACTTACTTGTCTGACGGTGCTGAGATTCAAGAGA |
| | | AGGGATTCTTCTACGGTTACACTTACTATGTTTGGTTCGTTATCTTCT |
| | | TGGCTTCCGTTGGTGGTTTGTACACTTCCGTTGTTGTTAAGTACACT |
| | | GACAACATCATGAAGGGATTCTCTGCTGCTGCTGCTATTGTTTTGTC |
| | | CACTATCGCTTCCGTTTTGTTGTTCGGATTGCAGATCACATTGTCCTT |
| | | TGCTTTGGGAGCTTTGTTGGTTTGTGTTTCCATCTACTTGTACGGATT |
| | | GCCAAGACAAGACACTACTTCCATTCAGCAAGAGGCTACTTCCAAG |
| | | GAGAGAATCATCGGTGTTTAGTAG |
| 96 | Human UDP- | ATGGAAAAGAACGGTAACAACAGAAAGTTGAGAGTTTGTGTTGCTA |
| | GlcNAc 2- | CTTGTAACAGAGCTGACTACTCCAAGTTGGCTCCAATCATGTTCGGT |
| | epimemse/N- | ATCAAGACTGAGCCAGAGTTCTTCGAGTTGGACGTTGTTGTTTTGGG |
| | acetylmannosamine | TTCCCACTTGATTGATGACTACGGTAACACTTACAGAATGATCGAG |
| | kinase (HsGNE) | CAGGACGACTTCGACATCAACACTAGATTGCACACTATTGTTAGAG |
| | codon opitimized | GAGAGGACGAAGCTGCTATGGTTGAATCTGTTGGATTGGCTTTGGT |
| | | TAAGTTGCCAGACGTTTTGAACAGATTGAAGCCAGACATCATGATT |
| | | GTTCACGGTGACAGATTCGATGCTTTGGCTTTGGCTACTTCCGCTGC |
| | | TTTGATGAACATTAGAATCTTGCACATCGAGGGTGGTGAAGTTTCTG |
| | | GTACTATCGACGACTCCATCAGACACGCTATCACTAAGTTGGCTCAC |
| | | TACCATGTTTGTTGTACTAGATCCGCTGAGCAACACTTGATTTCCAT |
| | | GTGTGAGGACCACGACAGAATTTTGTTGGCTGGTTGTCCATCTTACG |
| | | ACAAGTTGTTGTCCGCTAAGAACAAGGACTACATGTCCATCATCAG |
| | | AATGTGGTTGGGTGACGACGTTAAGTCTAAGGACTACATCGTTGCT |
| | | TTGCAGCACCCAGTTACTACTGACATCAAGCACTCCATCAAGATGTT |
| | | CGAGTTGACTTTGGACGCTTTGATCTCCTTCAACAAGAGAACTTTGG |
| | | TTTTGTTCCCAAACATTGACGCTGGTTCCAAAGAGATGGTTAGAGTT |
| | | ATGAGAAAGAAGGGTATCGAACACCACCCAAACTTCAGAGCTGTTA |
| | | AGCACGTTCCATTCGACCAATTCATCCAGTTGGTTGCTCATGCTGGT |
| | | TGTATGATCGGTAACTCCTCCTGTGGTGTTAGAGAAGTTGGTGCTTT |
| | | CGGTACTCCAGTTATCAACTTGGGTACTAGACAGATCGGTAGAGAG |
| | | ACTGGAGAAAACGTTTTGCATGTTAGAGATGCTGACACTCAGGACA |
| | | AGATTTTGCAGGCTTTGCACTTGCAATTCGGAAAGCAGTACCCATGT |
| | | TCCAAAATCTACGGTGACGGTAACGCTGTTCCAAGAATCTTGAAGT |
| | | TTTTGAAGTCCATCGACTTGCAAGAGCCATTGCAGAAGAAGTTCTG |
| | | TTTCCCACCAGTTAAGGAGAACATCTCCCAGGACATTGACCACATCT |
| | | TGGAGACATTGTCCGCTTTGGCTGTTGATTTGGGTGGAACTAACTTG |
| | | AGAGTTGCTATCGTTTCCATGAAGGGAGAGATCGTTAAGAAGTACA |
| | | CTCAGTTCAACCCAAAGACTTACGAGGAGAGAATCAACTTGATCTT |
| | | GCAGATGTGTGTTGAAGCTGCTGCTGAGGCTGTTAAGTTGAACTGT |
| | | AGAATCTTGGGTGTTGGTATCTCTACTGGTGGTAGAGTTAATCCAAG |
| | | AGAGGGTATCGTTTTGCACTCCACTAAGTTGATTCAGGAGTGGAAC |
| | | TCCGTTGATTTGAGAACTCCATTGTCCGACACATTGCACTTGCCAGT |
| | | TTGGGTTGACAACGACGGTAATTGTGCTGCTTTGGCTGAGAGAAAG |
| | | TTCGGTCAAGGAAAGGGATTGGAGAACTTCGTTACTTTGATCACTG |
| | | GTACTGGTATTGGTGGTGGTATCATTCACCAGCACGAGTTGATTCAC |
| | | GGTTCTTCCTTCTGTGCTGCTGAATTGGGACACTTGGTTGTTTCTTTG |
| | | GACGGTCCAGACTGTTCTTGTGGTTCCCACGGTTGTATTGAAGCTTA |
| | | CGCATCAGGAATGGCATTGCAGAGAGAGGCTAAGAAGTTGCACGA |
| | | CGAGGACTTGTTGTTGGTTGAGGGAATGTCTGTTCCAAAGGACGAG |
| | | GCTGTTGGTGCTTTGCATTTGATCCAGGCTGCTAAGTTGGGTAATGC |
| | | TAAGGCTCAGTCCATCTTGAGAACTGCTGGTACTGCTTTGGGATTGG |
| | | GTGTTGTTAATATCTTGCACACTATGAACCCATCCTTGGTTATCTTGT |
| | | CCGGTGTTTTGGCTTCTCACTACATCCACATCGTTAAGGACGTTATC |
| | | AGACAGCAAGCTTTGTCCTCCGTTCAAGACGTTGATGTTGTTGTTTC |
| | | CGACTTGGTTGACCCAGCTTTGTTGGGTGCTGCTTCCATGGTTTTGG |
| | | ACTACACTACTAGAAGAATCTACTAATAG |
| 97 | Sequence of the | CAGTTGAGCCAGACCGCGCTAAACGCATACCAATTGCCAAATCAGG |
| | PpARG 1 | CAATTGTGAGACAGTGGTAAAAAAGATGCCTGCAAAGTTAGATTCA |
| | auxotrophic marker: | CACAGTAAGAGAGATCCTACTCATAAATGAGGCGCTTATTTAGTAG |
| | | CTAGTGATAGCCACTGCGGTTCTGCTTTATGCTATTTGTTGTATGCCT |
| | | TACTATCTTTGTTTGGCTCCTTTTTCTTGACGTTTTCCGTTGGAGGGA |
| | | CTCCCTATTCTGAGTCATGAGCCGCACAGATTATCGCCCAAAATTGA |
| | | CAAAATCTTCTGGCGAAAAAAGTATAAAAGGAGAAAAAAGCTCAC |
| | | CCTTTTCCAGCGTAGAAAGTATATATCAGTCATTGAAGACTATTATT |
| | | TAAATAACACAATGTCTAAAGGAAAAGTTTGTTTGGCCTACTCCGG |
| | | TGGTTTGGATACCTCCATCATCCTAGCTTGGTTGTTGGAGCAGGGAT |
| | | ACGAAGTCGTTGCCTTTTTAGCCAACATTGGTCAAGAGGAAGACTT |
| | | TGAGGCTGCTAGAGAGAAAGCTCTGAAGATCGGTGCTACCAAGTTT |
| | | ATCGTCAGTGACGTTAGGAAGGAATTTGTTGAGGAAGTTTTGTTCCC |
| | | AGCAGTCCAAGTTAACGCTATCTACGAGAACGTCTACTTACTGGGT |
| | | ACCTCTTTGGCCAGACCAGTCATTGCCAAGGCCCAAATAGAGGTTG |
| | | CTGAACAAGAAGGTTGTTTTGCTGTTGCCCACGGTTGTACCGGAAA |
| | | GGGTAACGATCAGGTTAGATTTGAGCTTTCCTTTTATGCTCTGAAGC |
| | | CTGACGTTGTCTGTATCGCCCCATGGAGAGACCCAGAATTCTTCGAA |
| | | AGATTCGCTGGTAGAAATGACTTGCTGAATTACGCTGCTGAGAAGG |
| | | ATATTCCAGTTGCTCAGACTAAAGCCAAGCCATGGTCTACTGATGA |
| | | GAACATGGCTCACATCTCCTTCGAGGCTGGTATTCTAGAAGATCCA |
| | | AACACTACTCCTCCAAAGGACATGTGGAAGCTCACTGTTGACCCAG |
| | | AAGATGCACCAGACAAGCCAGAGTTCTTTGACGTCCACTTTGAGAA |
| | | GGGTAAGCCAGTTAAATTAGTTCTCGAGAACAAAACTGAGGTCACC |
| | | GATCCGGTTGAGATCTTTTTGACTGCTAACGCCATTGCTAGAAGAA |
| | | ACGGTGTTGGTAGAATTGACATTGTCGAGAACAGATTCATCGGAAT |
| | | CAAGTCCAGAGGTTGTTATGAAACTCCAGGTTTGACTCTACTGAGA |
| | | ACCACTCACATCGACTTGGAAGGTCTTACCGTTGACCGTGAAGTTA |
| | | GATCGATCAGAGACACTTTTGTTACCCCAACCTACTCTAAGTTGTTA |
| | | TACAACGGGTTGTACTTTACCCCAGAAGGTGAGTACGTCAGAACTA |
| | | TGATTCAGCCTTCTCAAAACACCGTCAACGGTGTTGTTAGAGCCAA |
| | | GGCCTACAAAGGTAATGTGTATAACCTAGGAAGATACTCTGAAACC |
| | | GAGAAATTGTACGATGCTACCGAATCTTCCATGGATGAGTTGACCG |
| | | GATTCCACCCTCAAGAAGCTGGAGGATTTATCACAACACAAGCCAT |
| | | CAGAATCAAGAAGTACGGAGAAAGTGTCAGAGAGAAGGGAAAGTT |
| | | TTTGGGACTTTAACTCAAGTAAAAGGATAGTTGTACAATTATATATA |
| | | CGAAGAATAAATCATTACAAAAAGTATTCGTTTCTTTGATTCTTAAC |
| | | AGGATTCATTTTCTGGGTGTCATCAGGTACAGCGCTGAATATCTTGA |
| | | AGTTAACATCGAGCTCATCATCGACGTTCATCACACTAGCCACGTTT |
| | | CCGCAACGGTAGCAATAATTAGGAGCGGACCACACAGTGACGACA |
| | | TC |
| 98 | Human CMP-sialic | ATGGACTCTGTTGAAAAGGGTGCTGCTACTTCTGTTTCCAACCCAAG |
| | acid synthase | AGGTAGACCATCCAGAGGTAGACCTCCTAAGTTGCAGAGAAACTCC |
| | (HsCSS) codon | AGAGGTGGTCAAGGTAGAGGTGTTGAAAAGCCACCACACTTGGCTG |
| | optimized | CTTTGATCTTGGCTAGAGGAGGTTCTAAGGGTATCCCATTGAAGAA |
| | | CATCAAGCACTTGGCTGGTGTTCCATTGATTGGATGGGTTTTGAGAG |
| | | CTGCTTTGGACTCTGGTGCTTTCCAATCTGTTTGGGTTTCCACTGACC |
| | | ACGACGAGATTGAGAACGTTGCTAAGCAATTCGGTGCTCAGGTTCA |
| | | CAGAAGATCCTCTGAGGTTTCCAAGGACTCTTCTACTTCCTTGGACG |
| | | CTATCATCGAGTTCTTGAACTACCACAACGAGGTTGACATCGTTGGT |
| | | AACATCCAAGCTACTTCCCCATGTTTGCACCCAACTGACTTGCAAAA |
| | | AGTTGCTGAGATGATCAGAGAAGAGGGTTACGACTCCGTTTTCTCC |
| | | GTTGTTAGAAGGCACCAGTTCAGATGGTCCGAGATTCAGAAGGGTG |
| | | TTAGAGAGGTTACAGAGCCATTGAACTTGAACCCAGCTAAAAGACC |
| | | AAGAAGGCAGGATTGGGACGGTGAATTGTACGAAAACGGTTCCTTC |
| | | TACTTCGCTAAGAGACACTTGATCGAGATGGGATACTTGCAAGGTG |
| | | GAAAGATGGCTTACTACGAGATGAGAGCTGAACACTCCGTTGACAT |
| | | CGACGTTGATATCGACTGGCCAATTGCTGAGCAGAGAGTTTTGAGA |
| | | TACGGTTACTTCGGAAAGGAGAAGTTGAAGGAGATCAAGTTGTTGG |
| | | TTTGTAACATCGACGGTTGTTTGACTAACGGTCACATCTACGTTTCT |
| | | GGTGACCAGAAGGAGATTATCTCCTACGACGTTAAGGACGCTATTG |
| | | GTATCTCCTTGTTGAAGAAGTCCGGTATCGAAGTTAGATTGATCTCC |
| | | GAGAGAGCTTGTTCCAAGCAAACATTGTCCTCTTTGAAGTTGGACT |
| | | GTAAGATGGAGGTTTCCGTTTCTGACAAGTTGGCTGTTGTTGACGAA |
| | | TGGAGAAAGGAGATGGGTTTGTGTTGGAAGGAAGTTGCTTACTTGG |
| | | GTAACGAAGTTTCTGACGAGGAGTGTTTGAAGAGAGTTGGTTTGTC |
| | | TGGTGCTCCAGCTGATGCTTGTTCCACTGCTCAAAAGGCTGTTGGTT |
| | | ACATCTGTAAGTGTAACGGTGGTAGAGGTGCTATTAGAGAGTTCGC |
| | | TGAGCACATCTGTTTGTTGATGGAGAAAGTTAATAACTCCTGTCAG |
| | | AAGTAGTAG |
| 99 | Human N- | ATGCCATTGGAATTGGAGTTGTGTCCTGGTAGATGGGTTGGTGGTC |
| | acetylneuraminate-9- | AACACCCATGTTTCATCATCGCTGAGATCGGTCAAAACCACCAAGG |
| | phosphate synthase | AGACTTGGACGTTGCTAAGAGAATGATCAGAATGGCTAAGGAATGT |
| | (HsSPS) codon | GGTGCTGACTGTGCTAAGTTCCAGAAGTCCGAGTTGGAGTTCAAGT |
| | optimized | TCAACAGAAAGGCTTTGGAAAGACCATACACTTCCAAGCACTCTTG |
| | | GGGAAAGACTTACGGAGAACACAAGAGACACTTGGAGTTCTCTCAC |
| | | GACCAATACAGAGAGTTGCAGAGATACGCTGAGGAAGTTGGTATCT |
| | | TCTTCACTGCTTCTGGAATGGACGAAATGGCTGTTGAGTTCTTGCAC |
| | | GAGTTGAACGTTCCATTCTTCAAAGTTGGTTCCGGTGACACTAACAA |
| | | CTTCCCATACTTGGAAAAGACTGCTAAGAAAGGTAGACCAATGGTT |
| | | ATCTCCTCTGGAATGCAGTCTATGGACACTATGAAGCAGGTTTACC |
| | | AGATCGTTAAGCCATTGAACCCAAACTTTTGTTTCTTGCAGTGTACT |
| | | TCCGCTTACCCATTGCAACCAGAGGACGTTAATTTGAGAGTTATCTC |
| | | CGAGTACCAGAAGTTGTTCCCAGACATCCCAATTGGTTACTCTGGTC |
| | | ACGAGACTGGTATTGCTATTTCCGTTGCTGCTGTTGCTTTGGGTGCT |
| | | AAGGTTTTGGAGAGACACATCACTTTGGACAAGACTTGGAAGGGTT |
| | | CTGATCACTCTGCTTCTTTGGAACCTGGTGAGTTGGCTGAACTTGTT |
| | | AGATCAGTTAGATTGGTTGAGAGAGCTTTGGGTTCCCCAACTAAGC |
| | | AATTGTTGCCATGTGAGATGGCTTGTAACGAGAAGTTGGGAAAGTC |
| | | CGTTGTTGCTAAGGTTAAGATCCCAGAGGGTACTATCTTGACTATGG |
| | | ACATGTTGACTGTTAAAGTTGGAGAGCCAAAGGGTTACCCACCAGA |
| | | GGACATCTTTAACTTGGTTGGTAAAAAGGTTTTGGTTACTGTTGAGG |
| | | AGGACGACACTATTATGGAGGAGTTGGTTGACAACCACGGAAAGA |
| | | AGATCAAGTCCTAG |
| 100 | Mouse alpha-2,6- | GTTTTTCAAATGCCAAAGTCCCAGGAGAAAGTTGCTGTTGGTCCAG |
| | sialyl transferase | CTCCACAAGCTGTTTTCTCCAACTCCAAGCAAGATCCAAAGGAGGG |
| | catalytic domain | TGTTCAAATCTTGTCCTACCCAAGAGTTACTGCTAAGGTTAAGCCAC |
| | (MmmST6) codon | AACCATCCTTGCAAGTTTGGGACAAGGACTCCACTTACTCCAAGTTG |
| | optimized | AACCCAAGATTGTTGAAGATTTGGAGAAACTACTTGAACATGAACA |
| | | AGTACAAGGTTTCCTACAAGGGTCCAGGTCCAGGTGTTAAGTTCTC |
| | | CGTTGAGGCTTTGAGATGTCACTTGAGAGACCACGTTAACGTTTCCA |
| | | TGATCGAGGCTACTGACTTCCCATTCAACACTACTGAATGGGAGGG |
| | | ATACTTGCCAAAGGAGAACTTCAGAACTAAGGCTGGTCCATGGCAT |
| | | AAGTGTGCTGTTGTTTCTTCTGCTGGTTCCTTGAAGAACTCCCAGTT |
| | | GGGTAGAGAAATTGACAACCACGACGCTGTTTTGAGATTCAACGGT |
| | | GCTCCAACTGACAACTTCCAGCAGGATGTTGGTACTAAGACTACTA |
| | | TCAGATTGGTTAACTCCCAATTGGTTACTACTGAGAAGAGATTCTTG |
| | | AAGGACTCCTTGTACACTGAGGGAATCTTGATTTTGTGGGACCCATC |
| | | TGTTTACCACGCTGACATTCCACAATGGTATCAGAAGCCAGACTAC |
| | | AACTTCTTCGAGACTTACAAGTCCTACAGAAGATTGCACCCATCCCA |
| | | GCCATTCTACATCTTGAAGCCACAAATGCCATGGGAATTGTGGGAC |
| | | ATCATCCAGGAAATTTCCCCAGACTTGATCCAACCAAACCCACCAT |
| | | CTTCTGGAATGTTGGGTATCATCATCATGATGACTTTGTGTGACCAG |
| | | GTTGACATCTACGAGTTCTTGCCATCCAAGAGAAAGACTGATGTTT |
| | | GTTACTACCACCAGAAGTTCTTCGACTCCGCTTGTACTATGGGAGCT |
| | | TACCACCCATTGTTGTTCGAGAAGAACATGGTTAAGCACTTGAACG |
| | | AAGGTACTGACGAGGACATCTACTTGTTCGGAAAGGCTACTTTGTC |
| | | CGGTTTCAGAAACAACAGATGTTAG |
| 101 | Pp TRP2: 5′ and | ACTGGGCCTTTAGAGGGTGCTGAAGTTGACCCCTTGGTGCTTCTGGA |
| | ORF | AAAAGAACTGAAGGGCACCAGACAAGCGCAACTTCCTGGTATTCCT |
| | | CGTCTAAGTGGTGGTGCCATAGGATACATCTCGTACGATTGTATTAA |
| | | GTACTTTGAACCAAAAACTGAAAGAAAACTGAAAGATGTTTTGCAA |
| | | CTTCCGGAAGCAGCTTTGATGTTGTTCGACACGATCGTGGCTTTTGA |
| | | CAATGTTTATCAAAGATTCCAGGTAATTGGAAACGTTTCTCTATCCG |
| | | TTGATGACTCGGACGAAGCTATTCTTGAGAAATATTATAAGACAAG |
| | | AGAAGAAGTGGAAAAGATCAGTAAAGTGGTATTTGACAATAAAAC |
| | | TGTTCCCTACTATGAACAGAAAGATATTATTCAAGGCCAAACGTTC |
| | | ACCTCTAATATTGGTCAGGAAGGGTATGAAAACCATGTTCGCAAGC |
| | | TGAAAGAACATATTCTGAAAGGAGACATCTTCCAAGCTGTTCCCTC |
| | | TCAAAGGGTAGCCAGGCCGACCTCATTGCACCCTTTCAACATCTATC |
| | | GTCATTTGAGAACTGTCAATCCTTCTCCATACATGTTCTATATTGAC |
| | | TATCTAGACTTCCAAGTTGTTGGTGCTTCACCTGAATTACTAGTTAA |
| | | ATCCGACAACAACAACAAAATCATCACACATCCTATTGCTGGAACT |
| | | CTTCCCAGAGGTAAAACTATCGAAGAGGACGACAATTATGCTAAGC |
| | | AATTGAAGTCGTCTTTGAAAGACAGGGCCGAGCACGTCATGCTGGT |
| | | AGATTTGGCCAGAAATGATATTAACCGTGTGTGTGAGCCCACCAGT |
| | | ACCACGGTTGATCGTTTATTGACTGTGGAGAGATTTTCTCATGTGAT |
| | | GCATCTTGTGTCAGAAGTCAGTGGAACATTGAGACCAAACAAGACT |
| | | CGCTTCGATGCTTTCAGATCCATTTTCCCAGCAGGTACCGTCTCCGG |
| | | TGCTCCGAAGGTAAGAGCAATGCAACTCATAGGAGAATTGGAAGG |
| | | AGAAAAGAGAGGTGTTTATGCGGGGGCCGTAGGACACTGGTCGTAC |
| | | GATGGAAAATCGATGGACACATGTATTGCCTTAAGAACAATGGTCG |
| | | TCAAGGACGGTGTCGCTTACCTTCAAGCCGGAGGTGGAATTGTCTA |
| | | CGATTCTGACCCCTATGACGAGTACATCGAAACCATGAACAAAATG |
| | | AGATCCAACAATAACACCATCTTGGAGGCTGAGAAAATCTGGACCG |
| | | ATAGGTTGGCCAGAGACGAG |
| | | AATCAAAGTGAATCCGAAGAAAACGATCAATGA |
| 102 | PpTRP2 3′ region | ACGGAGGACGTAAGTAGGAATTTATGTAATCATGCCAATACATCTT |
| | | TAGATTTCTTCCTCTTCTTTTTAACGAAAGACCTCCAGTTTTGCACTC |
| | | TCGACTCTCTAGTATCTTCCCATTTCTGTTGCTGCAACCTCTTGCCTT |
| | | CTGTTTCCTTCAATTGTTCTTCTTTCTTCTGTTGCACTTGGCCTTCTTC |
| | | CTCCATCTTTCGTTTTTTTTCAAGCCTTTTCAGCAGTTCTTCTTCCAA |
| | | GAGCAGTTCTTTGATTTTCTCTCTCCAATCCACCAAAAAACTGGATG |
| | | AATTCAACCGGGCATCATCAATGTTCCACTTTCTTTCTCTTATCAAT |
| | | AATCTACGTGCTTCGGCATACGAGGAATCCAGTTGCTCCCTAATCGA |
| | | GTCATCCACAAGGTTAGCATGGGCCTTTTTCAGGGTGTCAAAAGCA |
| | | TCTGGAGCTCGTTTATTCGGAGTCTTGTCTGGATGGATCAGCAAAGA |
| | | CTTTTTGCGGAAAGTCTTTCTTATATCTTCCGGAGAACAACCTGGTT |
| | | TCAAATCCAAGATGGCATAGCTGTCCAATTTGAAAGTGGAAAGAAT |
| | | CCTGCCAATTTCCTTCTCTCGTGTCAGCTCGTTCTCCTCCTTTTGCAA |
| | | CAGGTCCACTTCATCTGGCATTTTTCTTTATGTTAACTTTAATTATTA |
| | | TTAATTATAAAGTTGATTATCGTTATCAAAATAATCATATTCGAGAA |
| | | ATAATCCGTCCATGCAATATATAAATAAGAATTCATAATAATGTAA |
| | | TGATAACAGTACCTCTGATGACCTTTGATGAACCGCAATTTTCTTTC |
| | | CAATGACAAGACATCCCTATAATACAATTATACAGTTTATATATCAC |
| | | AAATAATCACCTTTTTATAAGAAAACCGTCCTCTCCGTAACAGAACT |
| | | TATTATCCGCACGTTATGGTTAACACACTACTAATACCGATATAGTG |
| | | TATGAAGTCGCTACGAGATAGCCATCCAGGAAACTTACCAATTCAT |
| | | CAGCACTTTCATGATCCGATTGTTGGCTTTATTCTTTGCGAGACAGA |
| | | TACTTGCCAATGAAATAACTGATCCCACAGATGAGAATCCGGTGCT |
| | | CGT |
| 103 | DNA encodes Tr | CGCGCCGGATCTCCCAACCCTACGAGGGCGGCAGCAGTCAAGGCCG |
| | ManI catalytic | CATTCCAGACGTCGTGGAACGCTTACCACCATTTTGCCTTTCCCCAT |
| | domain | GACGACCTCCACCCGGTCAGCAACAGCTTTGATGATGAGAGAAACG |
| | | GCTGGGGCTCGTCGGCAATCGATGGCTTGGACACGGCTATCCTCAT |
| | | GGGGGATGCCGACATTGTGAACACGATCCTTCAGTATGTACCGCAG |
| | | ATCAACTTCACCACGACTGCGGTTGCCAACCAAGGCATCTCCGTGTT |
| | | CGAGACCAACATTCGGTACCTCGGTGGCCTGCTTTCTGCCTATGACC |
| | | TGTTGCGAGGTCCTTTCAGCTCCTTGGCGACAAACCAGACCCTGGTA |
| | | AACAGCCTTCTGAGGCAGGCTCAAACACTGGCCAACGGCCTCAAGG |
| | | TTGCGTTCACCACTCCCAGCGGTGTCCCGGACCCTACCGTCTTCTTC |
| | | AACCCTACTGTCCGGAGAAGTGGTGCATCTAGCAACAACGTCGCTG |
| | | AAATTGGAAGCCTGGTGCTCGAGTGGACACGGTTGAGCGACCTGAC |
| | | GGGAAACCCGCAGTATGCCCAGCTTGCGCAGAAGGGCGAGTCGTAT |
| | | CTCCTGAATCCAAAGGGAAGCCCGGAGGCATGGCCTGGCCTGATTG |
| | | GAACGTTTGTCAGCACGAGCAACGGTACCTTTCAGGATAGCAGCGG |
| | | CAGCTGGTCCGGCCTCATGGACAGCTTCTACGAGTACCTGATCAAG |
| | | ATGTACCTGTACGACCCGGTTGCGTTTGCACACTACAAGGATCGCTG |
| | | GGTCCTTGCTGCCGACTCGACCATTGCGCATCTCGCCTCTCACCCGT |
| | | CGACGCGCAAGGACTTGACCTTTTTGTCTTCGTACAACGGACAGTCT |
| | | ACGTCGCCAAACTCAGGACATTTGGCCAGTTTTGCCGGTGGCAACTT |
| | | CATCTTGGGAGGCATTCTCCTGAACGAGCAAAAGTACATTGACTTT |
| | | GGAATCAAGCTTGCCAGCTCGTACTTTGCCACGTACAACCAGACGG |
| | | CTTCTGGAATCGGCCCCGAAGGCTTCGCGTGGGTGGACAGCGTGAC |
| | | GGGCGCCGGCGGCTCGCCGCCCTCGTCCCAGTCCGGGTTCTACTCGT |
| | | CGGCAGGATTCTGGGTGACGGCACCGTATTACATCCTGCGGCCGGA |
| | | GACGCTGGAGAGCTTGTACTACGCATACCGCGTCACGGGCGACTCC |
| | | AAGTGGCAGGACCTGGCGTGGGAAGCGTTCAGTGCCATTGAGGACG |
| | | CATGCCGCGCCGGCAGCGCGTACTCGTCCATCAACGACGTGACGCA |
| | | GGCCAACGGCGGGGGTGCCTCTGACGATATGGAGAGCTTCTGGTTT |
| | | GCCGAGGCGCTCAAGTATGCGTACCTGATCTTTGCGGAGGAGTCGG |
| | | ATGTGCAGGTGCAGGCCAACGGCGGGAACAAATTTGTCTTTAACAC |
| | | GGAGGCGCACCCCTTTAGCATCCGTTCATCATCACGACGGGGCGGC |
| | | CACCTTGCTTAA |
| 104 | Saccharomyces | ATGAGATTCCCATCCATCTTCACTGCTGTTTTGTTCGCTGCTTCTTCT |
| | cerevisiae mating | GCTTTGGCT |
| | factor pre-signal | |
| | peptide (DNA) | |
| 105 | Saccharomyces | MRFPSIFTAVLFAASSALA |
| | cerevisiae mating | |
| | factor pre-signal | |
| | peptide (protein) | |
| 106 | Sequence of the 5′- | TTGGGGGCCTCCAGGACTTGCTGAAATTTGCTGACTCATCTTCGCCA |
| | Region used for | TCCAAGGATAATGAGTTAGCTAATGTGACAGTTAATGAGTCGTCTT |
| | knock out of STE13 | GACTAACGGGGAACATTTCATTATTTATATCCAGAGTCAATTTGATA |
| | | GCAGAGTTTGTGGTTGAAATACCTATGATTCGGGAGACTTTGTTGTA |
| | | ACGACCATTATCCACAGTTTGGACCGTGAAAATGTCATCGAAGAGA |
| | | GCAGACGACATATTATCTATTGTGGTAAGTGATAGTTGGAAGTCCG |
| | | ACTAAGGCATGAAAATGAGAAGACTGAAAATTTAAAGTTTTTGAAA |
| | | ACACTAATCGGGTAATAACTTGGAAATTACGTTTACGTGCCTTTAGC |
| | | TCTTGTCCTTACCCCTGATAATCTATCCATTTCCCGAGAGACAATGA |
| | | CATCTCGGACAGCTGAGAACCCGTTCGATATAGAGCTTCAAGAGAA |
| | | TCTAAGTCCACGTTCTTCCAATTCGTCCATATTGGAAAACATTAATG |
| | | AGTATGCTAGAAGACATCGCAATGATTCGCTTTCCCAAGAATGTGA |
| | | TAATGAAGATGAGAACGAAAATCTCAATTATACTGATAACTTGGCC |
| | | AAGTTTTCAAAGTCTGGAGTATCAAGAAAGAGCTGTATGCTAATAT |
| | | TTGGTATTTGCTTTGTTATCTGGCTGTTTCTCTTTGCCTTGTATGCGA |
| | | GGGACAATCGATTTTCCAATTTGAACGAGTACGTTCCAGATTCAAA |
| | | CAG |
| 107 | Sequence of the 3′- | CTACTGGGAACCACGAGACATCACTGCAGTAGTTTCCAAGTGGATT |
| | Region used for | TCAGATCACTCATTTGTGAATCCTGACAAAACTGCGATATGGGGGT |
| | knock out of STE13 | GGTCTTACGGTGGGTTCACTACGCTTAAGACATTGGAATATGATTCT |
| | | GGAGAGGTTTTCAAATATGGTATGGCTGTTGCTCCAGTAACTAATTG |
| | | GCTTTTGTATGACTCCATCTACACTGAAAGATACATGAACCTTCCAA |
| | | AGGACAATGTTGAAGGCTACAGTGAACACAGCGTCATTAAGAAGGT |
| | | TTCCAATTTTAAGAATGTAAACCGATTCTTGGTTTGTCACGGGACTA |
| | | CTGATGATAACGTGCATTTTCAGAACACACTAACCTTACTGGACCA |
| | | GTTCAATATTAATGGTGTTGTGAATTACGATCTTCAGGTGTATCCCG |
| | | ACAGTGAACATAGCATTGCCCATCACAACGCAAATAAAGTGATCTA |
| | | CGAGAGGTTATTCAAGTGGTTAGAGCGGGCATTTAACGATAGATTT |
| | | TTGTAACATTCCGTACTTCATGCCATACTATATATCCTGCAAGGTTT |
| | | CCCTTTCAGACACAATAATTGCTTTGCAATTTTACATACCACCAATT |
| | | GGCAAAAATAATCTCTTCAGTAAGTTGAATGCTTTTCAAGCCAGCA |
| | | CCGTGAGAAATTGCTACAGCGCGCATTCTAACATCACTTTAAAATTC |
| | | CCTCGCCGGTGCTCACTGGAGTTTCCAACCCTTAGCTTATCAAAATC |
| | | GGGTGATAACTCTGAGTTTTTTTTTTCACTTCTATTCCTAAACCTTCG |
| | | CCCAATGCTACCACCTCCAATCAACATCCCGAAATGGATAGAAGAG |
| | | AATGGACATCTCTTGCAACCTCCGGTTAATAATTACTGTCTCCACAG |
| | | AGGAGGATTTACGGTAATGATTGTAGGTGGGCCTAATG |
| 108 | NatR ORF | ATGGGTACCACTCTTGACGACACGGCTTACCGGTACCGCACCAGTG |
| | | TCCCGGGGGACGCCGAGGCCATCGAGGCACTGGATGGGTCCTTCAC |
| | | CACCGACACCGTCTTCCGCGTCACCGCCACCGGGGACGGCTTCACC |
| | | CTGCGGGAGGTGCCGGTGGACCCGCCCCTGACCAAGGTGTTCCCCG |
| | | ACGACGAATCGGACGACGAATCGGACGACGGGGAGGACGGCGACC |
| | | CGGACTCCCGGACGTTCGTCGCGTACGGGGACGACGGCGACCTGGC |
| | | GGGCTTCGTGGTCGTCTCGTACTCCGGCTGGAACCGCCGGCTGACC |
| | | GTCGAGGACATCGAGGTCGCCCCGGAGCACCGGGGGCACGGGGTC |
| | | GGGCGCGCGTTGATGGGGCTCGCGACGGAGTTCGCCCGCGAGCGGG |
| | | GCGCCGGGCACCTCTGGCTGGAGGTCACCAACGTCAACGCACCGGC |
| | | GATCCACGCGTACCGGCGGATGGGGTTCACCCTCTGCGGCCTGGAC |
| | | ACCGCCCTGTACGACGGCACCGCCTCGGACGGCGAGCAGGCGCTCT |
| | | ACATGAGCATGCCCTGCCCCTAA |
| 109 | Ashbya gossypii | GATCTGTTTAGCTTGCCTCGTCCCCGCCGGGTCACCCGGCCAGCGAC |
| | TEF1 promoter | ATGGAGGCCCAGAATACCCTCCTTGACAGTCTTGACGTGCGCAGCT |
| | | CAGGGGCATGATGTGACTGTCGCCCGTACATTTAGCCCATACATCCC |
| | | CATGTATAATCATTTGCATCCATACATTTTGATGGCCGCACGGCGCG |
| | | AAGCAAAAATTACGGCTCCTCGCTGCAGACCTGCGAGCAGGGAAAC |
| | | GCTCCCCTCACAGACGCGTTGAATTGTCCCCACGCCGCGCCCCTGTA |
| | | GAGAAATATAAAAGGTTAGGATTTGCCACTGAGGTTCTTCTTTCATA |
| | | TACTTCCTTTTAAAATCTTGCTAGGATACAGTTCTCACATCACATCC |
| | | GAACATAAACAACC |
| 110 | Ashbya gossypii | TAATCAGTACTGACAATAAAAAGATTCTTGTTTTCAAGAACTTGTCA |
| | TEF1 termination | TTTGTATAGTTTTTTTATATTGTAGTTGTTCTATTTTAATCAAATGTT |
| | sequence | AGCGTGATTTATATTTTTTTTCGCCTCGACATCATCTGCCCAGATGC |
| | | GAAGTTAAGTGCGCAGAAAGTAATATCATGCGTCAATCGTATGTGA |
| | | ATGCTGGTCGCTATACTGCTGTCGATTCGATACTAACGCCGCCATCC |
| | | AGTGTCGAAAAC |
| 111 | Sequence of the 5′- | CACCTGGGCCTGTTGCTGCTGGTACTGCTGTTGGAACTGTTGGTATT |
| | Region used for | GTTGCTGATCTAAGGCCGCCTGTTCCACACCGTGTGTATCGAATGCT |
| | knock out of DAP2 | TGGGCAAAATCATCGCCTGCCGGAGGCCCCACTACCGCTTGTTCCTC |
| | | CTGCTCTTGTTTGTTTTGCTCATTGATGATATCGGCGTCAATGAATTG |
| | | ATCCTCAATCGTGTGGTGGTGGTGTCGTGATTCCTCTTCTTTCTTGAG |
| | | TGCCTTATCCATATTCCTATCTTAGTGTACCAATAATTTTGTTAAACA |
| | | CACGCTGTTGTTTATGAAAAGTCGTCAAAAGGTTAAAAATTCTACTT |
| | | GGTGTGTGTCAGAGAAAGTAGTGCAGACCCCCAGTTTGTTGACTAG |
| | | TTGAGAAGGCGGCTCACTATTGCGCGAATAGCATGAGAAATTTGCA |
| | | AACATCTGGCAAAGTGGTCAATACCTGCCAACCTGCCAATCTTCGC |
| | | GACGGAGGCTGTTAAGCGGGTTGGGTTCCCAAAGTGAATGGATATT |
| | | ACGGGCAGGAAAAACAGCCCCTTCCACACTAGTCTTTGCTACTGAC |
| | | ATCTTCCCTCTCATGTATCCCGAACACAAGTATCGGGAGTATCAACG |
| | | GAGGGTGCCCTTATGGCAGTACTCCCTGTTGGTGATTGTACTGCTAT |
| | | ACGGGTCTCATTTGCTTATCAGCACCATCAACTTGATACACTATAAC |
| | | CACAAAAATTATCATGCACACCCAGTCAATAGTGGTATCGTTCTTA |
| | | ATGAGTTTGCTGATGACGATTCATTCTCTTTGAATGGCACTCTGAAC |
| | | TTGGAGAACTGGAGAAATGGTACCTTTTCCCCTAAATTTCATTCCAT |
| | | TCAGTGGACCGAAATAGGTCAGGAAGATGACCAGGGATATTACATT |
| | | CTCTCTTCCAATTCCTCTTACATAGTAAAGTCTTTATCCGACCCAGA |
| | | CTTTGAATCTGTTCTATTCAACGAGTCTACAATCACTTACAACG |
| 112 | Sequence of the 3′- | GGCAGCAAAGCCTTACGTTGATGAGAATAGACTGGCCATTTGGGGT |
| | Region used for | TGGTCTTATGGAGGTTACATGACGCTAAAGGTTTTAGAACAGGATA |
| | knock out of DAP2 | AAGGTGAAACATTCAAATATGGAATGTCTGTTGCCCCTGTGACGAA |
| | | TTGGAAATTCTATGATTCTATCTACACAGAAAGATACATGCACACTC |
| | | CTCAGGACAATCCAAACTATTATAATTCGTCAATCCATGAGATTGAT |
| | | AATTTGAAGGGAGTGAAGAGGTTCTTGCTAATGCACGGAACTGGTG |
| | | ACGACAATGTTCACTTCCAAAATACACTCAAAGTTCTAGATTTATTT |
| | | GATTTACATGGTCTTGAAAACTATGATATCCACGTGTTCCCTGATAG |
| | | TGATCACAGTATTAGATATCACAACGGTAATGTTATAGTGTATGAT |
| | | AAGCTATTCCATTGGATTAGGCGTGCATTCAAGGCTGGCAAATAAA |
| | | TAGGTGCAAAAATATTATTAGACTTTTTTTTTCGTTCGCAAGTTATT |
| | | ACTGTGTACCATACCGATCCAATCCGTATTGTAATTCATGTTCTAGA |
| | | TCCAAAATTTGGGACTCTAATTCATGAGGTCTAGGAAGATGATCAT |
| | | CTCTATAGTTTTCAGCGGGGGGCTCGATTTGCGGTTGGTCAAAGCTA |
| | | ACATCAAAATGTTTGTCAGGTTCAGTGAATGGTAACTGCTGCTCTTG |
| | | AATTGGTCGTCTGACAAATTCTCTAAGTGATAGCACTTCATCTACAA |
| | | TCATTTGCTTCATCGTTTCTATATCGTCCACGACCTCAAACGAGAAA |
| | | TCGAATTTGGAAGAACAGACGGGCTCATCGTTAGGATCATGCCAAA |
| | | CCTTGAGATATGGATGCTCTAAAGCCTCAGTAACTGTAATTCTGTGA |
| | | GTGGGATCTACCGTGAGCATTCGATCCAGTAAGTCTATCGCTTCAG |
| | | GGTTGGCACCGGGAAATAACTGGCTGAATGGGATCTTGGGCATGAA |
| | | TGGCAGGGAGCGAACATAATCCTGGGCACGCTCTGATCTGATAGAC |
| | | TGAAGTGTCTCTTCCGAAACAGTACCCAGCGTACTCAAAATCAAGT |
| | | TCAATTGATCCACATAGTCTCTTCCTCTAAAAATGGGTCGGCCACCT |
| | | A |
| 113 | HYG^{R} resistance | GATCTGTTTAGCTTGCCTCGTCCCCGCCGGGTCACCCGGCCAGCGAC |
| | cassette | ATGGAGGCCCAGAATACCCTCCTTGACAGTCTTGACGTGCGCAGCT |
| | | CAGGGGCATGATGTGACTGTCGCCCGTACATTTAGCCCATACATCCC |
| | | CATGTATAATCATTTGCATCCATACATTTTGATGGCCGCACGGCGCG |
| | | AAGCAAAAATTACGGCTCCTCGCTGCGGACCTGCGAGCAGGGAAAC |
| | | GCTCCCCTCACAGACGCGTTGAATTGTCCCCACGCCGCGCCCCTGTA |
| | | GAGAAATATAAAAGGTTAGGATTTGCCACTGAGGTTCTTCTTTCATA |
| | | TACTTCCTTTTAAAATCTTGCTAGGATACAGTTCTCACATCACATCC |
| | | GAACATAAACAACCATGGGTAAAAAGCCTGAACTCACCGCGACGTC |
| | | TGTCGAGAAGTTTCTGATCGAAAAGTTCGACAGCGTCTCCGACCTG |
| | | ATGCAGCTCTCGGAGGGCGAAGAATCTCGTGCTTTCAGCTTCGATG |
| | | TAGGAGGGCGTGGATATGTCCTGCGGGTAAATAGCTGCGCCGATGG |
| | | TTTCTACAAAGATCGTTATGTTTATCGGCACTTTGCATCGGCCGCGC |
| | | TCCCGATTCCGGAAGTGCTTGACATTGGGGAATTCAGCGAGAGCCT |
| | | GACCTATTGCATCTCCCGCCGTGCACAGGGTGTCACGTTGCAAGAC |
| | | CTGCCTGAAACCGAACTGCCCGCTGTTCTGCAGCCGGTCGCGGAGG |
| | | CCATGGATGCGATCGCTGCGGCCGATCTTAGCCAGACGAGCGGGTT |
| | | CGGCCCATTCGGACCGCAAGGAATCGGTCAATACACTACATGGCGT |
| | | GATTTCATATGCGCGATTGCTGATCCCCATGTGTATCACTGGCAAAC |
| | | TGTGATGGACGACACCGTCAGTGCGTCCGTCGCGCAGGCTCTCGAT |
| | | GAGCTGATGCTTTGGGCCGAGGACTGCCCCGAAGTCCGGCACCTCG |
| | | TGCACGCGGATTTCGGCTCCAACAATGTCCTGACGGACAATGGCCG |
| | | CATAACAGCGGTCATTGACTGGAGCGAGGCGATGTTCGGGGATTCC |
| | | CAATACGAGGTCGCCAACATCTTCTTCTGGAGGCCGTGGTTGGCTTG |
| | | TATGGAGCAGCAGACGCGCTACTTCGAGCGGAGGCATCCGGAGCTT |
| | | GCAGGATCGCCGCGGCTCCGGGCGTATATGCTCCGCATTGGTCTTG |
| | | ACCAACTCTATCAGAGCTTGGTTGACGGCAATTTCGATGATGCAGC |
| | | TTGGGCGCAGGGTCGATGCGACGCAATCGTCCGATCCGGAGCCGGG |
| | | ACTGTCGGGCGTACACAAATCGCCCGCAGAAGCGCGGCCGTCTGGA |
| | | CCGATGGCTGTGTAGAAGTACTCGCCGATAGTGGAAACCGACGCCC |
| | | CAGCACTCGTCCGAGGGCAAAGGAATAATCAGTACTGACAATAAA |
| | | AAGATTCTTGTTTTCAAGAACTTGTCATTTGTATAGTTTTTTTATATT |
| | | GTAGTTGTTCTATTTTAATCAAATGTTAGCGTGATTTATATTTTTTTT |
| | | CGCCTCGACATCATCTGCCCAGATGCGAAGTTAAGTGCGCAGAAAG |
| | | TAATATCATGCGTCAATCGTATGTGAATGCTGGTCGCTATACTGCTG |
| | | TCGATTCGATACTAACGCCGCCATCCAGTGTCGAAAACGAGCT |
| 114 | Sequence of | ACGACGGCCAAATTCATGATACACACTCTGTTTCAGCTGGTTTGGAC |
| | PpTRP5 5′ | TACCCTGGAGTTGGTCCTGAATTGGCTGCCTGGAAAGCAAATGGTA |
| | integration fragment | GAGCCCAATTTTCCGCTGTAACTGATGCCCAAGCATTAGAGGGATT |
| | | CAAAATCCTGTCTCAATTGGAAGGGATCATTCCAGCACTAGAGTCT |
| | | AGTCATGCAATCTACGGCGCATTGCAAATTGCAAAGACTATGTCTT |
| | | CGGACCAGTCCTTAGTTATTAATGTATCTGGAAGGGGTGATAAGGA |
| | | CGTCCAGAGTGTAGCTGAGATTTTACCTAAATTGGGACCTCAAATT |
| | | GGATGGGATTTGCGTTTCAGCGAAGACATTACTAAAGAGTGA |
| 115 | Sequence of | TCGATAGCACAATATTCAACTTGACTGGGTGTTAAGAACTAAGAGC |
| | PpTRP5 3′ | TCTGGGAAACTTTGTATTTATTACTACCAACACAGTCAAATTATTGG |
| | integration fragment | ATGTGTTTTTTTTTCCAGTACATTTCACTGAGCAGTTTGTTATACTCG |
| | | GTCTTTAATCTCCATATACATGCAGATTGTAATACAGATCTGAACAG |
| | | TTTGATTCTGATTGATCTTGCCACCAATATTCTATTTTTGTATCAAGT |
| | | AACAGAGTCAATGATCATTGGTAACGTAACGGTTTTCGTGTATAGT |
| | | AGTTAGAGCCCATCTTGTAACCTCATTTCCTCCCATATTAAAGTATC |
| | | AGTGATTCGCTGGAACGATTAACTAAGAAAAAAAAAATATCTGCAC |
| | | ATACTCATCAGTCTGTAAATCTAAGTCAAAACTGCTGTATCCAATAG |
| | | AAATCGGGATATACCTGGATGTTTTTTCCACATAAACAAACGGGAG |
| | | TTCAGCTTACTTATGGTGTTGATGCAATTCAGTATGATCCTACCAAT |
| | | AAAACGAAACTTTGGGATTTTGGCTGTTTGAGGGATCAAAAGCTGC |
| | | ACCTTTACAAGATTGACGGATCGACCATTAGACCAAAGCAAATGGC |
| | | CACCAA |
| 116 | VPS10-1 3′ flanking | ACGACGACGAGGAGAATATCAATTTTGATTCCCGGTAGATAGCTCA |
| | | CCCACGGTCACACACACAAACACACATACACATTAACACACAGAGT |
| | | TATTAGTTAACAGAGAAAACTCTAACAAAGTATTTATTTTCGTTACG |
| | | TAATCCGACTTTTCTTTTTACCGTTTTCTATTGCTCCTCTCATTTGCCC |
| | | CTAAAAGTTGCTCCTCATTACTAAAATCACCACACCATGCTCGAATA |
| | | TGATGTTACTAAATGCAAATTGTAGTCGTGCCTCTTGTGGTAATACT |
| | | ATAGGGAATATCTCTCGATTACTCGATTCTGGTTAATTTTTTCTTTTT |
| | | TTATAGGGGAAGTTTTTTTTTCTTCCCCTTTCTCTCCAGTTTATTTATT |
| | | TACTAAGAAAATCCAACAGATACCAACCACCCAAAAAGATCCTAAA |
| | | CAGCCTGTTTTTGAGGAGTTTTTCAGCAGCTAAGCTTCATCAGTTTT |
| | | TTAATACTTAATTTATTGCCCTTCACTTTGTTTCTTGTGGCTTTTAAG |
| | | GCTCTCCGGAACAGCGGTTTCAAAATCAAATCTCAGTTATTTGTTTG |
| | | CTCCGCTTTGTCAGTTCAAAGATCATGGTTTCCGAAAACAAGAATC |
| | | AATCTTCGATTTTGATGGACAACTCCAAGAAGCTCTCTCCGAAGCCC |
| | | ATTTTGAATAACAAGAATGAACCGTTTGGCATCGGCGTCGATGGAC |
| | | TTCAACATCCTCAACCGACTTTATGCCGCACAGAATCGGAACTCTTG |
| | | TTCAACTTGAGCCAAGTCAATAAATCCCAAATAACTTTGGACGGTG |
| | | CAGTTACTCCACCTGCTGATGGTAATGGGAATGAAGCAAAAAGAGC |
| | | AAATCTCATCTCTTTTGATGTTCCATCGTCTCAAGTGAAACATAGAG |
| | | GGTCTATTAGTGCAAGGCCCTCGGCAGTGAATGTGTCCCAAATTAC |
| | | CGGGGCCCTTTCTCAATCCGGATCTTCTAGAAATCCCTACGATCAAA |
| | | CACAGTCACCTCCACCTAGCACTTACGCCTCCAGGCAGAACTCCAC |
| | | CCATGGAAATAATATCGATAGCTTGCAATATTTGGCAACAAGAGAT |
| | | CTTAGTGCTTTAAGGCTGGAAAGAGATGCTTCCGCACGAGAAGCTA |
| | | CCTCTTCTGCAGTGTCCACTCCTGTTCAGTTCGATGTACCCAAACAA |
| | | CATCATCTCCTTCATTTAGAACAAGACCCGACAAGGCCCATCC |
| 117 | VPS10-1 5′ flanking | AAGTGGGCCAGATTATATAAATATGGATCAACATGAAGCCTTGAAA |
| | region | GATTTCAAGGACAGGCTTAGGAATTACGAAAAAGTTTACGAGACTA |
| | | TTGACGACCAGGAGGAAGAGGAGAACGAACGGTACAATATTCAGT |
| | | ATCTGAAGATAATCAACGCAGGAAAGAAGATAGTCAGTTATAACAT |
| | | AAATGGGTATTTATCGTCCCACACCGTTTTTTATCTCCTGAATTTCA |
| | | ATCTTGCAGAACGTCAAATATGGTTGACGACGAATGGAGAGACAGA |
| | | GTATAACCTTCAAAATAGGATTGGAGGTGATTCCAAATTAAGCAAT |
| | | GAGGGATGGAAATTTGCCAAAGCATTGCCCAAGTTTATAGCACAGA |
| | | AAAGAAAAGAGTTTCAACTTAGACAGTTGACCAAACACTATATCGA |
| | | GACTCAAACGCCCATTGAAGACGTACCGTTGGAGGAGCACACCAAG |
| | | CCAGTCAAATATTCTGATCTGCATTTCCATGTTTGGTCATCGGCTTT |
| | | AAAGAGATCTACTCAATCAACAACATTTTTTCCATCGGAAAATTACT |
| | | CTCTGAAGCAATTCAGAACGTTGAATGATCTCTGTTGCGGATCACTG |
| | | GATGGTTTGACTGAACAAGAGTTCAAAAGTAAATACAAAGAAGAA |
| | | TACCAGAATTCTCAGACTGATAAACTGAGTTTCAGTTTCCCTGGTAT |
| | | CGGTGGGGAGTCTTATTTGGACGTGATCAACCGTTTGAGACCACTA |
| | | ATAGTTGAACTAGAAAGGTTGCCAGAACATGTCCTGGTCATTACCC |
| | | ACCGGGTCATAGTAAGGATTTTACTAGGATATTTCATGAATTTGGAT |
| | | AGAAATCTGTTGACAGATTTGGAAATTTTGCATGGGTATGTTTATTG |
| | | TATTGAGCCGAAACCTTATGGTTTAGACTTAAAGATCTGGCAGTAT |
| | | GATGAGGCGGACAACGAGTTTAATGAAGTTGATAAGCTGGAATTCA |
| | | TGAAAAGAAGAAGAAAATCGATCAACGTCAACACGACAGATTTCA |
| | | GAATGCAGTTAAACAAAGAGTTGCAACAGGACGCTCTCAATAATAG |
| | | TCCTGGTAATAATAGTCCGGGCGTATCATCTCTATCTTCATACTCGT |
| | | CGTCCTCTTCCCTTTCCGCTGACGGGAGCGAGGGAGAAACATTAAT |
| | | ACCACAAGTATCCCAGGCGGAGAGCTACAACTTTGAATTTAACTCT |
| | | CTTTCATCATCAGTTTCATCGTTGAAAAGGACGACATCTTCTTCCCA |
| | | ACATTTGAGCTCCAATCCTAGTTGTCTGAGCATGCATAATGCCTCAT |
| | | TGGACGAGAATGACGACGAACATTTAATAGACCCGGCTTCTACAGA |
| | | CGACAAGCTAAACATGGTATTACAGGACAAAACGCTAATTAAAAA |
| | | GCTCAAAAGTTTACTACTTGACGAGGCCGAAGGCTAGACAATCCAC |
| | | AGTTAATTTTGATACTGTACTTTATAACGAGTAACATACATATCTTA |
| | | TGTAATCATCTATGTCACGTCACGTGCGCGCGACATTATTCCGAGAA |
| | | CTTGCGCCCTGCTAGCTCCACTGTCAGAGTGATAACTTCCCCAAAAT |
| | | AGGATCCAACTGTTTCCAATTGCTTTTGGAAATGTGGATTGAAAGA |
| | | AACCTCATAGCGT |
| 118 | Pp AOX1 promoter | AACATCCAAAGACGAAAGGTTGAATGAAACCTTTTTGCCATCCGAC |
| | | ATCCACAGGTCCATTCTCACACATAAGTGCCAAACGCAACAGGAGG |
| | | GGATACACTAGCAGCAGACCGTTGCAAACGCAGGACCTCCACTCCT |
| | | CTTCTCCTCAACACCCACTTTTGCCATCGAAAAACCAGCCCAGTTAT |
| | | TGGGCTTGATTGGAGCTCGCTCATTCCAATTCCTTCTATTAGGCTAC |
| | | TAACACCATGACTTTATTAGCCTGTCTATCCTGGCCCCCCTGGCGAG |
| | | GTTCATGTTTGTTTATTTCCGAATGCAACAAGCTCCGCATTACACCC |
| | | GAACATCACTCCAGATGAGGGCTTTCTGAGTGTGGGGTCAAATAGT |
| | | TTCATGTTCCCCAAATGGCCCAAAACTGACAGTTTAAACGCTGTCTT |
| | | GGAACCTAATATGACAAAAGCGTGATCTCATCCAAGATGAACTAAG |
| | | TTTGGTTCGTTGAAATGCTAACGGCCAGTTGGTCAAAAAGAAACTT |
| | | CCAAAAGTCGGCATACCGTTTGTCTTGTTTGGTATTGATTGACGAAT |
| | | GCTCAAAAATAATCTCATTAATGCTTAGCGCAGTCTCTCTATCGCTT |
| | | CTGAACCCCGGTGCACCTGTGCCGAAACGCAAATGGGGAAACACCC |
| | | GCTTTTTGGATGATTATGCATTGTCTCCACATTGTATGCTTCCAAGA |
| | | TTCTGGTGGGAATACTGCTGATAGCCTAACGTTCATGATCAAAATTT |
| | | AACTGTTCTAACCCCTACTTGACAGCAATATATAAACAGAAGGAAG |
| | | CTGCCCTGTCTTAAACCTTTTTTTTTATCATCATTATTAGCTTACTTT |
| | | CATAATTGCGACTGGTTCCAATTGACAAGCTTTTGATTTTAACGACT |
| | | TTTAACGACAACTTGAGAAGATCAAAAAACAACTAATTATTCGAAA |
| | | CG |
| 119 | Sequence of the 5′- | GAAGGGCCATCGAATTGTCATCGTCTCCTCAGGTGCCATCGCTGTGG |
| | region that was used | GCATGAAGAGAGTCAACATGAAGCGGAAACCAAAAAAGTTACAGC |
| | to knock into the | AAGTGCAGGCATTGGCTGCTATAGGACAAGGCCGTTTGATAGGACT |
| | PpPRO1 locus: | TTGGGACGACCTTTTCCGTCAGTTGAATCAGCCTATTGCGCAGATTT |
| | | TACTGACTAGAACGGATTTGGTCGATTACACCCAGTTTAAGAACGC |
| | | TGAAAATACATTGGAACAGCTTATTAAAATGGGTATTATTCCTATTG |
| | | TCAATGAGAATGACACCCTATCCATTCAAGAAATCAAATTTGGTGA |
| | | CAATGACACCTTATCCGCCATAACAGCTGGTATGTGTCATGCAGACT |
| | | ACCTGTTTTTGGTGACTGATGTGGACTGTCTTTACACGGATAACCCT |
| | | CGTACGAATCCGGACGCTGAGCCAATCGTGTTAGTTAGAAATATGA |
| | | GGAATCTAAACGTCAATACCGAAAGTGGAGGTTCCGCCGTAGGAAC |
| | | AGGAGGAATGACAACTAAATTGATCGCAGCTGATTTGGGTGTATCT |
| | | GCAGGTGTTACAACGATTATTTGCAAAAGTGAACATCCCGAGCAGA |
| | | TTTTGGACATTGTAGAGTACAGTATCCGTGCTGATAGAGTCGAAAA |
| | | TGAGGCTAAATATCTGGTCATCAACGAAGAGGAAACTGTGGAACAA |
| | | TTTCAAGAGATCAATCGGTCAGAACTGAGGGAGTTGAACAAGCTGG |
| | | ACATTCCTTTGCATACACGTTTCGTTGGCCACAGTTTTAATGCTGTT |
| | | AATAACAAAGAGTTTTGGTTACTCCATGGACTAAAGGCCAACGGAG |
| | | CCATTATCATTGATCCAGGTTGTTATAAGGCTATCACTAGAAAAAA |
| | | CAAAGCTGGTATTCTTCCAGCTGGAATTATTTCCGTAGAGGGTAATT |
| | | TCCATGAATACGAGTGTGTTGATGTTAAGGTAGGACTAAGAGATCC |
| | | AGATGACCCACATTCACTAGACCCCAATGAAGAACTTTACGTCGTT |
| | | GGCCGTGCCCGTTGTAATTACCCCAGCAATCAAATCAACAAAATTA |
| | | AGGGTCTACAAAGCTCGCAGATCGAGCAGGTTCTAGGTTACGCTGA |
| | | CGGTGAGTATGTTGTTCACAGGGACAACTTGGCTTTCCCAGTATTTG |
| | | CCGATCCAGAACTGTTGGATGTTGTTGAGAGTACCCTGTCTGAACA |
| | | GGAGAGAGAATCCAAACCAAATAAATAG |
| 120 | Sequence of the 3′- | AATTTCACATATGCTGCTTGATTATGTAATTATACCTTGCGTTCGAT |
| | region that was used | GGCATCGATTTCCTCTTCTGTCAATCGCGCATCGCATTAAAAGTATA |
| | to knock into the | CTTTTTTTTTTTTCCTATAGTACTATTCGCCTTATTATAAACTTTGCTA |
| | PpPRO1 locus: | GTATGAGTTCTACCCCCAAGAAAGAGCCTGATTTGACTCCTAAGAA |
| | | GAGTCAGCCTCCAAAGAATAGTCTCGGTGGGGGTAAAGGCTTTAGT |
| | | GAGGAGGGTTTCTCCCAAGGGGACTTCAGCGCTAAGCATATACTAA |
| | | ATCGTCGCCCTAACACCGAAGGCTCTTCTGTGGCTTCGAACGTCATC |
| | | AGTTCGTCATCATTGCAAAGGTTACCATCCTCTGGATCTGGAAGCGT |
| | | TGCTGTGGGAAGTGTGTTGGGATCTTCGCCATTAACTCTTTCTGGAG |
| | | GGTTCCACGGGCTTGATCCAACCAAGAATAAAATAGACGTTCCAAA |
| | | GTCGAAACAGTCAAGGAGACAAAGTGTTCTTTCTGACATGATTTCC |
| | | ACTTCTCATGCAGCTAGAAATGATCACTCAGAGCAGCAGTTACAAA |
| | | CTGGACAACAATCAGAACAAAAAGAAGAAGATGGTAGTCGATCTT |
| | | CTTTTTCTGTTTCTTCCCCCGCAAGAGATATCCGGCACCCAGATGTA |
| | | CTGAAAACTGTCGAGAAACATCTTGCCAATGACAGCGAGATCGACT |
| | | CATCTTTACAACTTCAAGGTGGAGATGTCACTAGAGGCATTTATCA |
| | | ATGGGTAACTGGAGAAAGTAGTCAAAAAGATAACCCGCCTTTGAAA |
| | | CGAGCAAATAGTTTTAATGATTTTTCTTCTGTGCATGGTGACGAGGT |
| | | AGGCAAGGCAGATGCTGACCACGATCGTGAAAGCGTATTCGACGA |
| | | GGATGATATCTCCATTGATGATATCAAAGTTCCGGGAGGGATGCGT |
| | | CGAAGTTTTTTATTACAAAAGCATAGAGACCAACAACTTTCTGGAC |
| | | TGAATAAAACGGCTCACCAACCAAAACAACTTACTAAACCTAATTT |
| | | CTTCACGAACAACTTTATAGAGTTTTTGGCATTGTATGGGCATTTTG |
| | | CAGGTGAAGATTTGGAGGAAGACGAAGATGAAGATTTAGACAGTG |
| | | GTTCCGAATCAGTCGCAGTCAGTGATAGTGAGGGAGAATTCAGTGA |
| | | GGCTGACAACAATTTGTTGTATGATGAAGAGTCTCTCCTATTAGCAC |
| | | CTAGTACCTCCAACTATGCGAGATCAAGAATAGGAAGTATTCGTAC |
| | | TCCTACTTATGGATCTTTCAGTTCAAATGTTGGTTCTTCGTCTATTCA |
| | | TCAGCAGTTAATGAAAAGTCAAATCCCGAAGCTGAAGAAACGTGG |
| | | ACAGCACAAGCATAAAACACAATCAAAAATACGCTCGAAGAAGCA |
| | | AACTACCACCGTAAAAGCAGTGTTGCTGCTATTAAA |
| 121 | Leishmania major | ATGGGTAAAAGAAAGGGAAACTCCTTGGGAGATTCTGGTTCTGCTG |
| | STT3D (DNA) | CTACTGCTTCCAGAGAGGCTTCTGCTCAAGCTGAAGATGCTGCTTCC |
| | | CAGACTAAGACTGCTTCTCCACCTGCTAAGGTTATCTTGTTGCCAAA |
| | | GACTTTGACTGACGAGAAGGACTTCATCGGTATCTTCCCATTTCCAT |
| | | TCTGGCCAGTTCACTTCGTTTTGACTGTTGTTGCTTTGTTCGTTTTGG |
| | | CTGCTTCCTGTTTCCAGGCTTTCACTGTTAGAATGATCTCCGTTCAA |
| | | ATCTACGGTTACTTGATCCACGAATTTGACCCATGGTTCAACTACAG |
| | | AGCTGCTGAGTACATGTCTACTCACGGATGGAGTGCTTTTTTCTCCT |
| | | GGTTCGATTACATGTCCTGGTATCCATTGGGTAGACCAGTTGGTTCT |
| | | ACTACTTACCCAGGATTGCAGTTGACTGCTGTTGCTATCCATAGAGC |
| | | TTTGGCTGCTGCTGGAATGCCAATGTCCTTGAACAATGTTTGTGTTT |
| | | TGATGCCAGCTTGGTTTGGTGCTATCGCTACTGCTACTTTGGCTTTCT |
| | | GTACTTACGAGGCTTCTGGTTCTACTGTTGCTGCTGCTGCAGCTGCT |
| | | TTGTCCTTCTCCATTATCCCTGCTCACTTGATGAGATCCATGGCTGGT |
| | | GAGTTCGACAACGAGTGTATTGCTGTTGCTGCTATGTTGTTGACTTT |
| | | CTACTGTTGGGTTCGTTCCTTGAGAACTAGATCCTCCTGGCCAATCG |
| | | GTGTTTTGACAGGTGTTGCTTACGGTTACATGGCTGCTGCTTGGGGA |
| | | GGTTACATCTTCGTTTTGAACATGGTTGCTATGCACGCTGGTATCTC |
| | | TTCTATGGTTGACTGGGCTAGAAACACTTACAACCCATCCTTGTTGA |
| | | GAGCTTACACTTTGTTCTACGTTGTTGGTACTGCTATCGCTGTTTGTG |
| | | TTCCACCAGTTGGAATGTCTCCATTCAAGTCCTTGGAGCAGTTGGGA |
| | | GCTTTGTTGGTTTTGGTTTTCTTGTGTGGATTGCAAGTTTGTGAGGTT |
| | | TTGAGAGCTAGAGCTGGTGTTGAAGTTAGATCCAGAGCTAATTTCA |
| | | AGATCAGAGTTAGAGTTTTCTCCGTTATGGCTGGTGTTGCTGCTTTG |
| | | GCTATCTCTGTTTTGGCTCCAACTGGTTACTTTGGTCCATTGTCTGTT |
| | | AGAGTTAGAGCTTTGTTTGTTGAGCACACTAGAACTGGTAACCCATT |
| | | GGTTGACTCCGTTGCTGAACATCAACCAGCTTCTCCAGAGGCTATGT |
| | | GGGCTTTCTTGCATGTTTGTGGTGTTACTTGGGGATTGGGTTCCATT |
| | | GTTTTGGCTGTTTCCACTTTCGTTCACTACTCCCCATCTAAGGTTTTC |
| | | TGGTTGTTGAACTCCGGTGCTGTTTACTACTTCTCCACTAGAATGGC |
| | | TAGATTGTTGTTGTTGTCCGGTCCAGCTGCTTGTTTGTCCACTGGTAT |
| | | CTTCGTTGGTACTATCTTGGAGGCTGCTGTTCAATTGTCTTTCTGGG |
| | | ACTCCGATGCTACTAAGGCTAAGAAGCAGCAAAAGCAGGCTCAAA |
| | | GACACCAAAGAGGTGCTGGTAAAGGTTCTGGTAGAGATGACGCTAA |
| | | GAACGCTACTACTGCTAGAGCTTTCTGTGACGTTTTCGCTGGTTCTT |
| | | CTTTGGCTTGGGGTCACAGAATGGTTTTGTCCATTGCTATGTGGGCT |
| | | TTGGTTACTACTACTGCTGTTTCCTTCTTCTCCTCCGAATTTGCTTCT |
| | | CACTCCACTAAGTTCGCTGAACAATCCTCCAACCCAATGATCGTTTT |
| | | CGCTGCTGTTGTTCAGAACAGAGCTACTGGAAAGCCAATGAACTTG |
| | | TTGGTTGACGACTACTTGAAGGCTTACGAGTGGTTGAGAGACTCTA |
| | | CTCCAGAGGACGCTAGAGTTTTGGCTTGGTGGGACTACGGTTACCA |
| | | AATCACTGGTATCGGTAACAGAACTTCCTTGGCTGATGGTAACACTT |
| | | GGAACCACGAGCACATTGCTACTATCGGAAAGATGTTGACTTCCCC |
| | | AGTTGTTGAAGCTCACTCCCTTGTTAGACACATGGCTGACTACGTTT |
| | | TGATTTGGGCTGGTCAATCTGGTGACTTGATGAAGTCTCCACACATG |
| | | GCTAGAATCGGTAACTCTGTTTACCACGACATTTGTCCAGATGACCC |
| | | ATTGTGTCAGCAATTCGGTTTCCACAGAAACGATTACTCCAGACCA |
| | | ACTCCAATGATGAGAGCTTCCTTGTTGTACAACTTGCACGAGGCTG |
| | | GAAAAAGAAAGGGTGTTAAGGTTAACCCATCTTTGTTCCAAGAGGT |
| | | TTACTCCTCCAAGTACGGACTTGTTAGAATCTTCAAGGTTATGAACG |
| | | TTTCCGCTGAGTCTAAGAAGTGGGTTGCAGACCCAGCTAACAGAGT |
| | | TTGTCACCCACCTGGTTCTTGGATTTGTCCTGGTCAATACCCACCTG |
| | | CTAAAGAAATCCAAGAGATGTTGGCTCACAGAGTTCCATTCGACCA |
| | | GGTTACAAACGCTGACAGAAAGAACAATGTTGGTTCCTACCAAGAG |
| | | GAATACATGAGAAGAATGAGAGAGTCCGAGAACAGAAGATAATAG |
| 122 | Sequence of the Sh | ATGGCCAAGTTGACCAGTGCCGTTCCGGTGCTCACCGCGCGCGACG |
| | ble ORF (Zeocin | TCGCCGGAGCGGTCGAGTTCTGGACCGACCGGCTCGGGTTCTCCCG |
| | resistance marker): | GGACTTCGTGGAGGACGACTTCGCCGGTGTGGTCCGGGACGACGTG |
| | | ACCCTGTTCATCAGCGCGGTCCAGGACCAGGTGGTGCCGGACAACA |
| | | CCCTGGCCTGGGTGTGGGTGCGCGGCCTGGACGAGCTGTACGCCGA |
| | | GTGGTCGGAGGTCGTGTCCACGAACTTCCGGGACGCCTCCGGGCCG |
| | | GCCATGACCGAGATCGGCGAGCAGCCGTGGGGGCGGGAGTTCGCC |
| | | CTGCGCGACCCGGCCGGCAACTGCGTGCACTTCGTGGCCGAGGAGC |
| | | AGGACTGA |
| 123 | ScTEF1 promoter | GATCCCCCACACACCATAGCTTCAAAATGTTTCTACTCCTTTTTTACT |
| | | CTTCCAGATTTTCTCGGACTCCGCGCATCGCCGTACCACTTCAAAAC |
| | | ACCCAAGCACAGCATACTAAATTTCCCCTCTTTCTTCCTCTAGGGTG |
| | | TCGTTAATTACCCGTACTAAAGGTTTGGAAAAGAAAAAAGAGACCG |
| | | CCTCGTTTCTTTTTCTTCGTCGAAAAAGGCAATAAAAATTTTTATCA |
| | | CGTTTCTTTTTCTTGAAAATTTTTTTTTTTGATTTTTTTCTCTTTCGAT |
| | | GACCTCCCATTGATATTTAAGTTAATAAACGGTCTTCAATTTCTCAA |
| | | GTTTCAGTTTCATTTTTCTTGTTCTATTACAACTTTTTTTACTTCTTGC |
| | | TCATTAGAAAGAAAGCATAGCAATCTAATCTAAGTTTTAATTACAA |
| | | A |
| 124 | PpAOX1 5′ flanking | GGCTTGGCCATAATTTTGACATTCGAGTCATCAAAGGTAAATTCAA |
| | region | CCGGAGACTTGTATTCTTTATTGATAACTTTCTCATATAGGACATTG |
| | | TCAGGAACACGATGAAACCAGGATGCCCCCAAATCCAATGAGACTG |
| | | AGGTTTCATGAGTCGCAACCAACCTACCTCCAATACGGTCCCTACCC |
| | | TCTAAAATCAACGCATTCACGCCATTGCTTTTGAGATCGACTGCAGC |
| | | TTTGATGCCTGAAATCCCAGCGCCTACAATGATGACATTTGGATTTG |
| | | GTTGACTCATGTTGGTATTGTGAAATAGACGCAGATCGGGAACACT |
| | | GAAAAATAACAGTTATTATTCGAGATCTAACATCCAAAGACGAAAG |
| | | GTTGAATGAAACCTTTTTGCCATCCGACATCCACAGGTCCATTCTCA |
| | | CACATAAGTGCCAAACGCAACAGGAGGGGATACACTAGCAGCAGA |
| | | CCGTTGCAAACGCAGGACCTCCACTCCTCTTCTCCTCAACACCCACT |
| | | TTTGCCATCGAAAAACCAGCCCAGTTATTGGGCTTGATTGGAGCTC |
| | | GCTCATTCCAATTCCTTCTATTAGGCTACTAACACCATGACTTTATT |
| | | AGCCTGTCTATCCTGGCCCCCCTGGCGAGGTTCATGTTTGTTTATTTC |
| | | CGAATGCAACAAGCTCCGCATTACACCCGAACATCACTCCAGATGA |
| | | GGGCTTTCTGAGTGTGGGGTCAAATAGTTTCATGTTCCCCAAATGGC |
| | | CCAAAACTGACAGTTTAAACGCTGTCTTGGAACCTAATATGACAAA |
| | | AGCGTGATCTCATCCAAGATGAACTAAGTTTGGTTCGTTGAAATGCT |
| | | AACGGCCAGTTGGTCAAAAAGAAACTTCCAAAAGTCGGCATACCGT |
| | | TTGTCTTGTTTGGTATTGATTGACGAATGCTCAAAAATAATCTCATT |
| | | AATGCTTAGCGCAGTCTCTCTATCGCTTCTGAACCCCGGTGCACCTG |
| | | TGCCGAAACGCAAATGGGGAAACACCCGCTTTTTGGATGATTATGC |
| | | ATTGTCTCCACATTGTATGCTTCCAAGATTCTGGTGGGAATACTGCT |
| | | GATAGCCTAACGTTCATGATCAAAATTTAACTGTTCTAACCCCTACT |
| | | TGACAGCAATATATAAACAGAAGGAAGCTGCCCTGTCTTAAACCTT |
| | | TTTTTTTATCATCATTATTAGCTTACTTTCATAATTGCGACTGGTTCC |
| | | AATTGACAAGCTTTTGATTTTAACGACTTTTAACGACAACTTGAGAA |
| | | GATCAAAAAACAACTAATTATTCGAAACGATGGCTATCCCCGAAGA |
| | | GTTTCTTGGCCATAATTTTGACATTCGAGTCATCAAAGGTAAATTCA |
| | | ACCGGAGACTTGTATTCTTTATTGATAACTTTCTCATATAGGACATT |
| | | GTCAGGAACACGATGAAACCAGGATGCCCCCAAATCCAATGAGACT |
| | | GAGGTTTCATGAGTCGCAACCAACCTACCTCCAATACGGTCCCTACC |
| | | CTCTAAAATCAACGCATTCACGCCATTGCTTTTGAGATCGACTGCAG |
| | | CTTTGATGCCTGAAATCCCAGCGCCTACAATGATGACATTTGGATTT |
| | | GGTTGACTCATGTTGGTATTGTGAAATAGACGCAGATCGGGAACAC |
| | | TGAAAAATAACAGTTATTATTCGAGATCTAACATCCAAAGACGAAA |
| | | GGTTGAATGAAACCTTTTTGCCATCCGACATCCACAGGTCCATTCTC |
| | | ACACATAAGTGCCAAACGCAACAGGAGGGGATACACTAGCAGCAG |
| | | ACCGTTGCAAACGCAGGACCTCCACTCCTCTTCTCCTCAACACCCAC |
| | | TTTTGCCATCGAAAAACCAGCCCAGTTATTGGGCTTGATTGGAGCTC |
| | | GCTCATTCCAATTCSTTCTATTAGGCTACTAACACCATGACTTTATTA |
| | | GCCTGTCTATCCTGGCCCCCCTGGCGAGGTTCATGTTTGTTTATTTCC |
| | | GAATGCAACAAGCTCCGCATTACACCCGAACATCACTCCAGATGAG |
| | | GGCTTTCTGAGTGTGGGGTCAAATAGTTTCATGTTCCCCAAATGGCC |
| | | CAAAACTGACAGTTTAAACGCTGTCTTGGAACCTAATATGACAAAa |
| | | GCGTGATCTCATCcaAGATGaACTAAGTTTGGWTCGtTGAAATGCTAA |
| | | CGgcCAGtTgGTCaAAAAGAAMCtTcCAAARGTCGGCATAcCGttTGTCT |
| | | TGtKTGGtAtTGAtTGACgaATGCTCAAAWATaaYCTcATTaATSCTTAGC |
| | | SSAtSYCTCTCTATYGCTTCTGAACCCCGGTGCACCTGTGCCGAAACG |
| | | CAAATGGGGAAACACCCGCTTTTTGGATGATTATGCATTGTCTCCAC |
| | | ATTGTATGCTTCCAAGATTCTGGTGGGAATACTGCTGATAGCCTAAC |
| | | GTTCATGATCAAAATTTAACTGTTCTAACCCCTACTTGACAGCAATA |
| | | TATAAACAGAAGGAAGCTGCCCTGTCTTAAACCTTTTTTTTTATCAT |
| | | CATTATTAGCTTACTTTCATAATTGCGACTGGTTCCAATTGACAAGC |
| | | TTTTGATTTTAACGACTTTTAACGACAACTTGAGAAGATCAAAAAA |
| | | CAACTAATTATTCGAAACGATGGCTATCCCCGAAGAGTTT |
| 125 | PpAOX1 3′ flanking | TCAAGAGGATGTCAGAATGCCATTTGCCTGAGAGATGCAGGCTTCA |
| | region | TTTTTGATACTTTTTTATTTGTAACCTATATAGTATAGGATTTTTTTT |
| | | GTCATTTTGTTTCTTCTCGTACGAGCTTGCTCCTGATCAGCCTATCTC |
| | | GCAGCTGATGAATATCTTGTGGTAGGGGTTTGGGAAAATCATTCGA |
| | | GTTTGATGTTTTTCTTGGTATTTCCCACTCCTCTTCAGAGTACAGAAG |
| | | ATTAAGTGAGACGTTCGTTTGTGCAAGCTTCAACGATGCCAAAAGG |
| | | GTATAATAAGCGTCATTTGCAGCATTGTGAAGAAAACTATGTGGCA |
| | | AGCCAAGCCTGCGAAGAATGTATTTTAAGTTTGACTTTGATGTATTC |
| | | ACTTGATTAAGCCATAATTCTCGAGTATCTATGATTGGAAGTATGGG |
| | | AATGGTGATACCCGCATTCTTCAGTGTCTTGAGGTCTCCTATCAGAT |
| | | TATGCCCAACTAAAGCAACCGGAGGAGGAGATTTCATGGTAAATTT |
| | | CTCTGACTTTTGGTCATCAGTAGACTCGAACTGTGAGACTATCTCGG |
| | | TTATGACAGCAGAAATGTCCTTCTTGGAGACAGTAAATGAAGTCCC |
| | | ACCAATAAAGAAATCCTTGTTATCAGGAACAAACTTCTTGTTTCGA |
| | | ACTTTTTCGGTGCCTTGAACTATAAAATGTAGAGTGGATATGTCGGG |
| | | TAGGAATGGAGCGGGCAAATGCTTACCTTCTGGACCTTCAAGAGGT |
| | | ATGTAGGGTTTGTAGATACTGATGCCAACTTCAGTGACAACGTTGCT |
| | | ATTTCGTTCAAACCATTCCGAATCCAGAGAAATCAAAGTTGTTTGTC |
| | | TACTATTGATCCAAGCCAGTGCGGTCTTGAAACTGACAATAGTGTG |
| | | CTCGTGTTTTGAGGTCATCTTTGTATGAATAAATCTAGTCTTTGATCT |
| | | AAATAATCTTGACGAGCCAGACGATAATACCAATCTAAACTCTTTA |
| | | AACGTTAAAGGACAAGTATGTCTGCCTGTATTAAACCCCAAATCAG |
| | | CTCGTAGTCTGATCCTCATCAACTTGAGGGGCACTATCTTGTTTTAG |
| | | AGAAATTTGCGGAGATGCGATATCGAGAAAAAGGTACGCTGATTTT |
| | | AAACGTGAAATTTATCTCAAGATCTATGTACATTAGGGCAAAACAG |
| | | CTAATCTATTTGGTTCTAGTAAGAACACTGTTAGTCACAAATTCTAA |
| | | TACCGAACGGGCTCCACTTTCGGGAAGCGTTCGTAAAGCTTCAAGT |
| | | GCTTGATCTCTATATTTACTGGCCAACACACGAGTCTTCTCAACCCC |
| | | GTCATTCTTTATAACGGCCGTTTTGGCAGTCTCAACATCACCAGGCT |
| | | TTGAGAAATTACGTGCTATCAGAGGTCCGAGACTGGGGTCATTTTTC |
| | | CAAGCATAGAGAATTCAAGAGGATGTCAGAATGCCATTTGCCTGAG |
| | | AGATGCAGGCTTCATTTTTGATACTTTTTTATTTGTAACCTATATAGT |
| | | ATAGGATTTTTTTTGTCATTTTGTTTCTTCTCGTACGAGCTTGCTCCT |
| | | GATTAGCCTATCTCGCAGCTGATGAATATCTTGTGGTAGGGGTTTGG |
| | | GAAAATCATTCGAGTTTGATGTTTTTCTTGGTATTTCCCACTCCTCTT |
| | | CAGAGTACAGAAGATTAAGTGAGACGTTCGTTTGTGCAAGCTTCAA |
| | | CGATGCCAAAAGGGTATAATAAGCGTCATTTGCAGCATTGTGAAGA |
| | | AAACTATGTGGCAAGCCAAGCCTGCGAAGAATGTATTTTAAGTTTG |
| | | ACTTTGATGTATTCACTTGATTAAGCCATAATTCTCGAGTATCTATG |
| | | ATTGGAAGTATGGGAATGGTGATACCCGCATTCTTCAGTGTCTTGA |
| | | GGTCTCCTATCAGATTATGCCCAACTAAAGCAACCGGAGGAGGAGA |
| | | TTTCATGGTAAATTTCTCTGACTTTTGGTCATCAGTAGACTCGAACT |
| | | GTGAGACTATCTCGGTTATGACAGCAGAAATGTCCTTCTTGGAGAC |
| | | AGTAAATGAAGTCCCACCAATAAAGAAATCCTTGTTATCAGGAACA |
| | | AACTTCTTGTTTCGAACTTTTTCGGTGCCTTGAACTATAAAATGTAG |
| | | AGTGGATATGTCGGGTAGGAATGGGAGCGGGCAAATGCTTACCTTC |
| | | TTGACCCTTCAAGAGGTATGTAGGGTTTGTAGATACTGATGCCAACT |
| | | TTCAGTGACAACGTTGCTATTTCGTTCAAACCCATTCCGAATCCAGA |
| | | GAAATCAAAGTTTGTTTGTCTACTATTGATCCAAGCCAGTGCGGTCT |
| | | TGAAAACTGACAATAGTGTGCTCGTGTTTTGAGGTCATCTTTTGTAT |
| | | GAATAAATCTAGTCTTTTGATCTAAATAATCTTGACGAGCCAGACG |
| | | ATAATACCAATCTAAACTCTTTAAACGTTAAAGGACAAGTATGTCT |
| | | GCCTGTATTAAACCCCAAATCAGCTCGTAGTCTGATCCTCATCAACT |
| | | TGAGGGGCACTATCTTGTTTTAGAGAAATTTGCGGAGATGCGATAT |
| | | CGAGAAAAAGGTACGCTGATTTTAAACGTGAAATTTATCTCAAGAT |
| | | CTATGTACATTAGGGCAAAACAGCTAATCTATTTGGTTCTAGTAAG |
| | | AACACTGTTAGTCACAAATTCTAATACCGAACGGGCTCCACTTTCGG |
| | | GAAGCGTTCGTAAAGCTTCAAGTGCTTGATCTCTATATTTACTGGCC |
| | | AACACACGAGTCTTCTCAACCCCGTCATTCTTTATAACGGCCGTTTT |
| | | GGCAGTCTCAACATCACCAGGCTTTGAGAAATTACGTGCTATCAGA |
| | | GGTCCGAGACTGGGGTCATTTTTCCAAGCATAGAGAATGGCCGCTG |
| | | T |
| 126 | DNA encoding Pre- | ATGAGATTTCCTTCAATTTTTACTGCAGTTTTATTCGCAGCATCCTCC |
| | proinsulin analogue | GCATTAGCTGCTCCAGTCAACACTACAACAGAAGATGAAACGGCAC |
| | precursor: S.c. alpha | AAATTCCGGCTGAAGCTGTCATCGGTTACTCAGATTTAGAAGGGGA |
| | mating factor signal | TTTCGATGTTGCTGTTTTGCCATTTTCCAACAGCACAAATAACGGGT |
| | sequence and pro- | TATTGTTTATAAATACTACTATTGCCAGCATTGCTGCTAAAGAAGAA |
| | peptide + N-terminal | GGGGTATCTCTCGAGAAAAGGGAAGAGGCAGAAGCTGAGGCCGAA |
| | spacer + B chain | CCAAAGTTTGTTAACCAACATTTGTGTGGTTCACACCTTGTTGAGGC |
| | des(B30) + C- | TTTGTACCTTGTCTGCGGTGAAAGAGGATTTTTCTATACTCCTAAGG |
| | peptide “AAK” + A | CTGCCAAAGGAATTGTCGAGCAATGTTGCACATCTATCTGTTCCTTG |
| | chain | TACCAGCTTGAAAACTATTGCAATTAA |
| 127 | Pre-proinsulin | MRFPSIFTAVLFAASSALAAPVNTTTEDETAQIPAEAVIGYSDLEGDFD |
| | analogue precursor: | VAVLPFSNSTNNGLLFINTTIASIAAKEEGVSLEKREEAEAEAEPKFVNQ |
| | S.c. alpha mating | HLCGSHLVEALYLVCGERGFFYTPKAAKGIVEQCCTSICSLYQLENYC |
| | factor signal | N |
| | sequence and pro- | |
| | peptide + B chain | |
| | des(B30) + C- | |
| | peptide “AAK” + A | |
| | chain | |
| 128 | DNA encoding Pre- | ATGAGATTTCCTTCAATTTTTACTGCAGTTTTATTCGCAGCATCCTCC |
| | proinsulin analogue | GCATTAGCTGCTCCAGTCAACACTACAACAGAAGATGAAACGGCAC |
| | precursor: S.c. alpha | AAATTCCGGCTGAAGCTGTCATCGGTTACTCAGATTTAGAAGGGGA |
| | mating factor signal | TTTCGATGTTGCTGTTTTGCCATTTTCCAACAGCACAAATAACGGGT |
| | sequence and pro- | TATTGTTTATAAATACTACTATTGCCAGCATTGCTGCTAAAGAAGAA |
| | peptide + N-terminal | GGGGTATCTCTCGAGAAAAGGGAAGAGGCAGAAGCTGAGGCCGAA |
| | spacer + B chain | CCAAAGAACACTACATTCGTTAACCAACATTTGTGTGGTTCACACCT |
| | NTT(−2) des(B30) + | TGTTGAGGCTTTGTACCTTGTCTGCGGTGAAAGAGGATTTTTCTATA |
| | C-peptide “AAK” + | CCCCTAAGGCTGCCAAAGGAATTGTCGAGCAATGTTGCACTTCTATC |
| | A chain | TGTTCCTTGTACCAGCTTGAAAACTATTGCAATTAA |
| 129 | Pre-proinsulin | MRFPSIFTAVLFAASSALAAPVNTTTEDETAQIPAEAVIGYSDLEGDFD |
| | analogue precursor: | VAVLPFSNSTNNGLLFINTTIASIAAKEEGVSLEKREEAEAEAEPKNTTF |
| | S.c. alpha mating | VNQHLCGSHLVEALYLVCGERGFFYTPKAAKGIVEQCCTSICSLYQLE |
| | factor signal | NYCN |
| | sequence and pro- | |
| | peptide + N-terminal | |
| | spacer + B chain | |
| | NTT(−2) des(B30) + | |
| | C-peptide “AAK” + | |
| | A chain | |
| 130 | DNA encoding Pre- | ATGAGATTTCCTTCAATTTTTACTGCAGTTTTATTCGCAGCATCCTCC |
| | proinsulin analogue | GCATTAGCTGCTCCAGTCAACACTACAACAGAAGATGAAACGGCAC |
| | precursor: S.c. alpha | AAATTCCGGCTGAAGCTGTCATCGGTTACTCAGATTTAGAAGGGGA |
| | mating factor signal | TTTCGATGTTGCTGTTTTGCCATTTTCCAACAGCACAAATAACGGGT |
| | sequence and pro- | TATTGTTTATAAATACTACTATTGCCAGCATTGCTGCTAAAGAAGAA |
| | peptide + N-terminal | GGGGTATCTCTCGAGAAAAGGGAAGAGGCAGAAGCTGAGGCCGAA |
| | spacer + B chain | CCAAAGAACGGTACTTTCGTTAACCAACATTTGTGTGGATCACACCT |
| | NGT(−2) des(B30) + | TGTTGAGGCTTTGTACCTTGTCTGCGGTGAAAGAGGATTTTTCTATA |
| | C-peptide “AAK” + | CTCCTAAGGCTGCCAAAGGTATTGTCGAGCAATGTTGCACATCTATC |
| | A chain | TGTTCCTTGTACCAGCTTGAAAACTATTGCAATTAA |
| 131 | Pre-proinsulin | MRFPSIFTAVLFAASSALAAPVNTTTEDETAQIPAEAVIGYSDLEGDFD |
| | analogue precursor: | VAVLPFSNSTNNGLLFINTTIASIAAKEEGVSLEKREEAEAEAEPKNGTF |
| | S.c. alpha mating | VNQHLCGSHLVEALYLVCGERGFFYTPKAAKGIVEQCCTSICSLYQLE |
| | factor signal | NYCN |
| | sequence and pro- | |
| | peptide + N-terminal | |
| | spacer + B chain | |
| | NGT(−2) des(B30) + | |
| | C-peptide “AAK” + | |
| | A chain | |
| 132 | DNA encoding Pre- | ATGAGATTTCCTTCAATTTTTACTGCAGTTTTATTCGCAGCATCCTCC |
| | proinsulin analogue | GCATTAGCTGCTCCAGTCAACACTACAACAGAAGATGAAACGGCAC |
| | precursor: S.c. alpha | AAATTCCGGCTGAAGCTGTCATCGGTTACTCAGATTTAGAAGGGGA |
| | mating factor signal | TTTCGATGTTGCTGTTTTGCCATTTTCCAACAGCACAAATAACGGGT |
| | sequence and pro- | TATTGTTTATAAATACTACTATTGCCAGCATTGCTGCTAAAGAAGAA |
| | peptide + N-terminal | GGGGTATCTCTCGAGAAAAGGGAAGAGGCAGAAGCTGAGGCCGAA |
| | spacer + B chain | CCAAAGTTTGTTAACCAACATTTGTGTGGTTCACACCTTGTTGAGGC |
| | des(B30) + C- | TTTGTACCTTGTCTGCGGTGAAAGAGGATTTTTCTATACCCCTAAGG |
| | peptide “AAK” + A | CTGCCAAAAATACTACAGGAATTGTCGAGCAATGTTGCACTTCTAT |
| | chain NTT(−2) | CTGTTCCTTGTACCAGCTTGAAAACTATTGCAATTAA |
| 133 | Pre-proinsulin | MRFPSIFTAVLFAASSALAAPVNTTTEDETAQIPAEAVIGYSDLEGDFD |
| | analogue: S.c. alpha | VAVLPFSNSTNNGLLFINTTIASIAAKEEGVSLEKREEAEAEAEPKFVNQ |
| | mating factor signal | HLCGSHLVEALYLVCGERGFFYTPKAAKNTTGIVEQCCTSICSLYQLEN |
| | sequence and pro- | YCN |
| | peptide + N-terminal | |
| | spacer + B chain | |
| | des(B30) + C- | |
| | peptide “AAK” + A | |
| | chain NTT(−2) | |
| 134 | DNA encoding Pre- | ATGAGATTTCCTTCAATTTTTACTGCAGTTTTATTCGCAGCATCCTCC |
| | proinsulin analogue | GCATTAGCTGCTCCAGTCAACACTACAACAGAAGATGAAACGGCAC |
| | precursor: S.c. alpha | AAATTCCGGCTGAAGCTGTCATCGGTTACTCAGATTTAGAAGGGGA |
| | mating factor signal | TTTCGATGTTGCTGTTTTGCCATTTTCCAACAGCACAAATAACGGGT |
| | sequence and pro- | TATTGTTTATAAATACTACTATTGCCAGCATTGCTGCTAAAGAAGAA |
| | peptide + N-terminal | GGGGTATCTCTCGAGAAAAGGGAAGAGGCAGAAGCTGAGGCCGAA |
| | spacer + B chain | CCAAAGTTTGTTAACCAACATTTGTGTGGTTCACACCTTGTTGAGGC |
| | P28N + C-peptide | TTTGTACCTTGTCTGCGGTGAAAGAGGATTTTTCTATACTAATAAGA |
| | “AAK” + A chain | CAGCTGCCAAAGGAATTGTCGAGCAATGTTGCACTTCTATCTGTTCC |
| | | TTGTACCAGCTTGAAAACTATTGCAATTAA |
| 135 | Pre-proinsulin | MRFPSIFTAVLFAASSALAAPVNTTTEDETAQIPAEAVIGYSDLEGDFD |
| | analogue precursor: | VAVLPFSNSTNNGLLFINTTIASIAAKEEGVSLEKREEAEAEAEPKFVNQ |
| | S.c. alpha mating | HLCGSHLVEALYLVCGERGFFYTNKTAAKGIVEQCCTSICSLYQLENY |
| | factor signal | CN |
| | sequence and pro- | |
| | peptide + N-terminal | |
| | spacer + B chain | |
| | P28N + C-peptide | |
| | “AAK” + A chain | |
| 136 | DNA encoding Pre- | ATGAGATTTCCTTCAATTTTTACTGCAGTTTTATTCGCAGCATCCTCC |
| | proinsulin analogue | GCATTAGCTGCTCCAGTCAACACTACAACAGAAGATGAAACGGCAC |
| | precursor: S.c. alpha | AAATTCCGGCTGAAGCTGTCATCGGTTACTCAGATTTAGAAGGGGA |
| | mating factor signal | TTTCGATGTTGCTGTTTTGCCATTTTCCAACAGCACAAATAACGGGT |
| | sequence and pro- | TATTGTTTATAAATACTACTATTGCCAGCATTGCTGCTAAAGAAGAA |
| | peptide + N-terminal | GGGGTATCTCTCGAGAAAAGGGAAGAGGCAGAAGCTGAGGCCGAA |
| | spacer + B chain | CCAAAGAACACTACATTCGTTAACCAACATTTGTGTGGTTCACACCT |
| | NTT(−2) P28N + C- | TGTTGAGGCTTTGTACCTTGTCTGCGGTGAAAGAGGATTTTTCTATA |
| | peptide “AAK” + A | CCAACAAGACTGCTGCCAAAGGAATTGTCGAGCAATGTTGCACATC |
| | chain | TATCTGTTCCTTGTACCAGCTTGAAAACTATTGCAATTAA |
| 137 | Pre-proinsulin | MRFPSIFTAVLFAASSALAAPVNTTTEDETAQIPAEAVIGYSDLEGDFD |
| | analogue precursor: | VAVLPFSNSTNNGLLFINTTIASIAAKEEGVSLEKREEAEAEAEPKNTTF |
| | S.c. alpha mating | VNQHLCGSHLVEALYLVCGERGFFYTNKTAAKGIVEQCCTSICSLYQL |
| | factor signal | ENYCN |
| | sequence and pro- | |
| | peptide + N-terminal | |
| | spacer + B chain | |
| | NTT(−2) P28N + C- | |
| | peptide “AAK” + A | |
| | chain | |
| 138 | DNA encoding Pre- | ATGAGATTTCCTTCAATTTTTACTGCAGTTTTATTCGCAGCATCCTCC |
| | proinsulin analogue | GCATTAGCTGCTCCAGTCAACACTACAACAGAAGATGAAACGGCAC |
| | precursor: S.c. alpha | AAATTCCGGCTGAAGCTGTCATCGGTTACTCAGATTTAGAAGGGGA |
| | mating factor signal | TTTCGATGTTGCTGTTTTGCCATTTTCCAACAGCACAAATAACGGGT |
| | sequence and pro- | TATTGTTTATAAATACTACTATTGCCAGCATTGCTGCTAAAGAAGAA |
| | peptide + N-terminal | GGGGTATCTCTCGAGAAAAGGGAAGAGGCAGAAGCTGAGGCCGAA |
| | spacer + B chain | CCAAAGAACGGTACCTTTGTTAATCAACATTTGTGTGGATCACACCT |
| | NGT(−2) P28N + C- | TGTTGAGGCTTTGTACCTTGTCTGCGGTGAAAGAGGATTTTTCTATA |
| | peptide “AAK” + A | CTAACAAGACAGCTGCCAAAGGTATTGTCGAGCAATGTTGCACTTC |
| | chain | TATCTGTTCCTTGTACCAGCTTGAAAACTATTGCAATTAA |
| 139 | Pre-proinsulin | MRFPSIFTAVLFAASSALAAPVNTTTEDETAQIPAEAVIGYSDLEGDFD |
| | analogue precursor: | VAVLPFSNSTNNGLLFINTTIASIAAKEEGVSLEKREEAEAEAEPKNGTF |
| | S.c. alpha mating | VNQHLCGSHLVEALYLVCGERGFFYTNKTAAKGIVEQCCTSICSLYQL |
| | factor signal | ENYCN |
| | sequence and pro- | |
| | peptide + N-terminal | |
| | spacer + B chain | |
| | NGT(−2) P28N + C- | |
| | peptide “AAK” + A | |
| | chain | |
| 140 | DNA encoding Pre- | ATGAGATTTCCTTCAATTTTTACTGCAGTTTTATTCGCAGCATCCTCC |
| | proinsulin analogue | GCATTAGCTGCTCCAGTCAACACTACAACAGAAGATGAAACGGCAC |
| | precursor: S.c. alpha | AAATTCCGGCTGAAGCTGTCATCGGTTACTCAGATTTAGAAGGGGA |
| | mating factor signal | TTTCGATGTTGCTGTTTTGCCATTTTCCAACAGCACAAATAACGGGT |
| | sequence and pro- | TATTGTTTATAAATACTACTATTGCCAGCATTGCTGCTAAAGAAGAA |
| | peptide + N-terminal | GGGGTATCTCTCGAGAAAAGGGAAGAGGCAGAAGCTGAGGCCGAA |
| | spacer + B chain | CCAAAGTTTGTTAACCAACATTTGTGTGGTTCACACCTTGTTGAGGC |
| | P28N + C-peptide | TTTGTACCTTGTCTGCGGTGAAAGAGGATTTTTCTATACCAACAAGA |
| | “AAK” + A chain | CTGCTGCCAAAAATACTACAGGAATTGTCGAGCAATGTTGCACATC |
| | NTT(−2) | TATCTGTTCCTTGTACCAGCTTGAAAACTATTGCAATTAA |
| 141 | Pre-proinsulin | MRFPSIFTAVLFAASSALAAPVNTTTEDETAQIPAEAVIGYSDLEGDFD |
| | analogue precursor: | VAVLPFSNSTNNGLLFINTTIASIAAKEEGVSLEKREEAEAEAEPKFVNQ |
| | S.c. alpha mating | HLCGSHLVEALYLVCGERGFFYTNKTAAKNTTGIVEQCCTSICSLYQLE |
| | factor signal | NYCN |
| | sequence and pro- | |
| | peptide + N-terminal | |
| | spacer + B chain | |
| | P28N + C-peptide | |
| | “AAK” + A chain | |
| | NTT(−2) | |
| 142 | DNA encoding Pre- | ATGAGATTTCCTTCAATTTTTACTGCAGTTTTATTCGCAGCATCCTCC |
| | proinsulin analogue | GCATTAGCTGCTCCAGTCAACACTACAACAGAAGATGAAACGGCAC |
| | precursor: S.c. alpha | AAATTCCGGCTGAAGCTGTCATCGGTTACTCAGATTTAGAAGGGGA |
| | mating factor signal | TTTCGATGTTGCTGTTTTGCCATTTTCCAACAGCACAAATAACGGGT |
| | sequence and pro- | TATTGTTTATAAATACTACTATTGCCAGCATTGCTGCTAAAGAAGAA |
| | peptide + N-terminal | GGGGTATCTCTCGAGAAAAGGGAAGAGGCAGAAGCTGAGGCCGAA |
| | spacer + B chain | CCAAAGTTTGTTAACCAACATTTGTGTGGTTCACACCTTGTTGAGGC |
| | P28N des(B30) + C- | TTTGTACCTTGTCTGCGGTGAAAGAGGATTTTTCTATACTAATAAGG |
| | peptide “AAK” + A | CTGCCAAAGGAATTGTCGAGCAATGTTGCACATCTATCTGTTCCTTG |
| | chain | TACCAGCTTGAAAACTATTGCAATTAA |
| 143 | Pre-proinsulin | MRFPSIFTAVLFAASSALAAPVNTTTEDETAQIPAEAVIGYSDLEGDFD |
| | analogue precursor: | VAVLPFSNSTNNGLLFINTTIASIAAKEEGVSLEKREEAEAEAEPKFVNQ |
| | S.c. alpha mating | HLCGSHLVEALYLVCGERGFFYTNKAAKGIVEQCCTSICSLYQLENYC |
| | factor signal | N |
| | sequence and pro- | |
| | peptide + B chain | |
| | P28N des(B30) + C- | |
| | peptide “AAK” + A | |
| | chain | |
| 144 | DNA encoding Pre- | ATGAGATTTCCTTCAATTTTTACTGCAGTTTTATTCGCAGCATCCTCC |
| | proinsulin analogue | GCATTAGCTGCTCCAGTCAACACTACAACAGAAGATGAAACGGCAC |
| | precursor: S.c. alpha | AAATTCCGGCTGAAGCTGTCATCGGTTACTCAGATTTAGAAGGGGA |
| | mating factor signal | TTTCGATGTTGCTGTTTTGCCATTTTCCAACAGCACAAATAACGGGT |
| | sequence and pro- | TATTGTTTATAAATACTACTATTGCCAGCATTGCTGCTAAAGAAGAA |
| | peptide + N-terminal | GGGGTATCTCTCGAGAAAAGGGAAGAGGCAGAAGCTGAGGCCGAA |
| | spacer + B chain | CCAAAGAACGGTACTTTCGTTAACCAACATTTGTGTGGATCACACCT |
| | NGT(−2) des(B30) + | TGTTGAGGCTTTGTACCTTGTCTGCGGTGAAAGAGGATTTTTCTATA |
| | C-peptide “AAK” + | CTCCTAAGGCTGCCAAAAACGGTACAGGAATTGTCGAGCAATGTTG |
| | A chain NGT(−2) | CACCTCTATCTGTTCCTTGTACCAGCTTGAAAACTATTGCAATTAA |
| 145 | Pre-proinsulin | MRFPSIFTAVLFAASSALAAPVNTTTEDETAQIPAEAVIGYSDLEGDFD |
| | analogue precursor: | VAVLPFSNSTNNGLLFINTTIASIAAKEEGVSLEKREEAEAEAEPKNGTF |
| | S.c. alpha mating | VNQHLCGSHLVEALYLVCGERGFFYTPKAAKNGTGIVEQCCTSICSLY |
| | factor signal | QLENYCN |
| | sequence and pro- | |
| | peptide + N-terminal | |
| | spacer + B chain | |
| | NGT(−2) des(B30) + | |
| | C-peptide “AAK” + | |
| | A chain NGT(−2) | |
| 146 | DNA encoding Pre- | ATGAGATTTCCTTCAATTTTTACTGCAGTTTTATTCGCAGCATCCTCC |
| | proinsulin analogue | GCATTAGCTGCTCCAGTCAACACTACAACAGAAGATGAAACGGCAC |
| | precursor: S.c. alpha | AAATTCCGGCTGAAGCTGTCATCGGTTACTCAGATTTAGAAGGGGA |
| | mating factor signal | TTTCGATGTTGCTGTTTTGCCATTTTCCAACAGCACAAATAACGGGT |
| | sequence and pro- | TATTGTTTATAAATACTACTATTGCCAGCATTGCTGCTAAAGAAGAA |
| | peptide + N-terminal | GGGGTATCTCTCGAGAAAAGGGAAGAGGCAGAAGCTGAGGCCGAA |
| | spacer + B chain | CCAAAGAACGGTACATTCGTTAACCAACATTTGTGTGGATCACACC |
| | NGT(−2) P28N + C- | TTGTTGAGGCTTTGTACCTTGTCTGCGGTGAAAGAGGATTTTTCTAT |
| | peptide “AAK” + A | ACTAACAAGACAGCTGCCAAAAATGGTACCGGAATTGTCGAGCAAT |
| | chain NGT(−2) | GTTGCACTTCTATCTGTTCCTTGTACCAGCTTGAAAACTATTGCAAT |
| | TAA | |
| 147 | Pre-proinsulin | MRFPSIFTAVLFAASSALAAPVNTTTEDETAQIPAEAVIGYSDLEGDFD |
| | analogue precursor: | VAVLPFSNSTNNGLLFINTTIASIAAKEEGVSLEKREEAEAEAEPKNGTF |
| | S.c. alpha mating | VNQHLCGSHLVEALYLVCGERGFFYTNKTAAKNGTGIVEQCCTSICSL |
| | factor signal | YQLENYCN |
| | sequence and pro- | |
| | peptide + N-terminal | |
| | spacer + B chain | |
| | NGT(−2) P28N + C- | |
| | peptide “AAK” + A | |
| | chain NGT(−2) | |
| 148 | Sc alpha mating | MRFPSIFTAVLFAASSALAAPVNTTTEDETAQIPAEAVIGYSDLEGDFD |
| | factor signal | VAVLPFSNSTNNGLLFINTTIASIAAKEEGVSLEKR |
| | sequence and pro- | |
| | peptide | |
| 149 | N-terminal spacer | EEAEAEAEPK |
| 150 | Proinsulin | EEAEAEAEPKFVNQHLCGSHLVEALYLVCGERGFFYTPKAAKGIVEQC |
| | (des(B30)) analogue | CTSICSLYQLENYCN |
| | precursor with N- | |
| | terminal spacer and | |
| | C-peptide “AAK” | |
| 151 | Proinsulin (B: NTT | EEAEAEAEPKNTTFVNQHLCGSHLVEALYLVCGERGFFYTPKAAKGIV |
| | (−2) des(B30)) | EQCCTSICSLYQLENYCN |
| | analogue precursor | |
| | with N-terminal | |
| | spacer and C-peptide | |
| | “AAK” | |
| 152 | Proinsulin (B: NGT | EEAEAEAEPKNGTFVNQHLCGSHLVEALYLVCGERGFFYTPKAAKGIV |
| | (−2) des(B30)) | EQCCTSICSLYQLENYCN |
| | analogue precursor | |
| | with N-terminal | |
| | spacer and C-peptide | |
| | “AAK” | |
| 153 | Proinsulin (des(B30) | EEAEAEAEPKFVNQHLCGSHLVEALYLVCGERGFFYTPKAAKNTTGIV |
| | A: NTT(−2)) | EQCCTSICSLYQLENYCN |
| | analogue precursor | |
| | with N-terminal | |
| | spacer and C-peptide | |
| | “AAK” | |
| 154 | Proinsulin (B: P28N) | EEAEAEAEPKFVNQHLCGSHLVEALYLVCGERGFFYTNKTAAKGIVEQ |
| | analogue precursor | CCTSICSLYQLENYCN |
| | with N-terminal | |
| | spacer and C-peptide | |
| | “AAK” | |
| 155 | Proinsulin (B: NTT | EEAEAEAEPKNTTFVNQHLCGSHLVEALYLVCGERGFFYTNKTAAKGI |
| | (−2) B: P28N) analogue | VEQCCTSICSLYQLENYCN |
| | precursor with N- | |
| | terminal spacer and | |
| | C-peptide “AAK” | |
| 156 | Proinsulin (B: NGT | EEAEAEAEPKNGTFVNQHLCGSHLVEALYLVCGERGFFYTNKTAAKGI |
| | (−2) B: P28N) analogue | VEQCCTSICSLYQLENYCN |
| | precursor with N- | |
| | terminal spacer and | |
| | C-peptide “AAK” | |
| 157 | Proinsulin (B: P28N | EEAEAEAEPKFVNQHLCGSHLVEALYLVCGERGFFYTNKTAAKNTTGI |
| | A: NTT(−2)) | VEQCCTSICSLYQLENYCN |
| | analogue precursor | |
| | with N-terminal | |
| | spacer and C-peptide | |
| | “AAK” | |
| 158 | Proinsulin (B: P28N | EEAEAEAEPKFVNQHLCGSHLVEALYLVCGERGFFYTNKAAKGIVEQC |
| | des(B30)) analogue | CTSICSLYQLENYCN |
| | precursor with N- | |
| | terminal spacer and | |
| | C-peptide “AAK” | |
| 159 | Proinsulin (B: NGT | EEAEAEAEPKNGTFVNQHLCGSHLVEALYLVCGERGFFYTPKAAKNG |
| | (−2) des(B30) | TGIVEQCCTSICSLYQLENYCN |
| | A: NGT(−2)) | |
| | analogue precursor | |
| | with N-terminal | |
| | spacer and C-peptide | |
| | “AAK” | |
| 160 | Proinsulin (B: NGT | EEAEAEAEPKNGTFVNQHLCGSHLVEALYLVCGERGFFYTNKTAAKN |
| | (−2) B: P28N A: NGT | GTGIVEQCCTSICSLYQLENYCN |
| | (−2)) analogue | |
| | precursor with N- | |
| | terminal spacer and | |
| | C-peptide “AAK” | |
| 161 | B-chain peptide core | HLCGSHLVEALYLVCGERGFF |
| | sequence | |
| 255 | ScARR3 ORF | ATGTCAGAAGATCAAAAAAGTGAAAATTCCGTACCTTCTAAGGTTA |
| | | ATATGGTGAATCGCACCGATATACTGACTACGATCAAGTCATTGTC |
| | | ATGGCTTGACTTGATGTTGCCATTTACTATAATTCTCTCCATAATCAT |
| | | TGCAGTAATAATTTCTGTCTATGTGCCTTCTTCCCGTCACACTTTTGA |
| | | CGCTGAAGGTCATCCCAATCTAATGGGAGTGTCCATTCCTTTGACTG |
| | | TTGGTATGATTGTAATGATGATTCCCCCGATCTGCAAAGTTTCCTGG |
| | | GAGTCTATTCACAAGTACTTCTACAGGAGCTATATAAGGAAGCAAC |
| | | TAGCCCTCTCGTTATTTTTGAATTGGGTCATCGGTCCTTTGTTGATGA |
| | | CAGCATTGGCGTGGATGGCGCTATTCGATTATAAGGAATACCGTCA |
| | | AGGCATTATTATGATCGGAGTAGCTAGATGCATTGCCATGGTGCTA |
| | | ATTTGGAATCAGATTGCTGGAGGAGACAATGATCTCTGCGTCGTGC |
| | | TTGTTATTACAAACTCGCTTTTACAGATGGTATTATATGCACCATTG |
| | | CAGATATTTTACTGTTATGTTATTTCTCATGACCACCTGAATACTTCA |
| | | AATAGGGTATTATTCGAAGAGGTTGCAAAGTCTGTCGGAGTTTTTCT |
| | | CGGCATACCACTGGGAATTGGCATTATCATACGTTTGGGAAGTCTT |
| | | ACCATAGCTGGTAAAAGTAATTATGAAAAATACATTTTGAGATTTA |
| | | TTTCTCCATGGGCAATGATCGGATTTCATTACACTTTATTTGTTATTT |
| | | TTATTAGTAGAGGTTATCAATTTATCCACGAAATTGGTTCTGCAATA |
| | | TTGTGCTTTGTCCCATTGGTGCTTTACTTCTTTATTGCATGGTTTTTG |
| | | ACCTTCGCATTAATGAGGTACTTATCAATATCTAGGAGTGATACAC |
| | | AAAGAGAATGTAGCTGTGACCAAGAACTACTTTTAAAGAGGGTCTG |
| | | GGGAAGAAAGTCTTGTGAAGCTAGCTTTTCTATTACGATGACGCAA |
| | | TGTTTCACTATGGCTTCAAATAATTTTGAACTATCCCTGGCAATTGC |
| | | TATTTCCTTATATGGTAACAATAGCAAGCAAGCAATAGCTGCAACA |
| | | TTTGGGCCGTTGCTAGAAGTTCCAATTTTATTGATTTTGGCAATAGT |
| | | CGCGAGAATCCTTAAACCATATTATATATGGAACAATAGAAATTAA |
| 256 | URA6 region | CAAATGCAAGAGGACATTAGAAATGTGTTTGGTAAGAACATGAAGC |
| | | CGGAGGCATACAAACGATTCACAGATTTGAAGGAGGAAAACAAAC |
| | | TGCATCCACCGGAAGTGCCAGCAGCCGTGTATGCCAACCTTGCTCTC |
| | | AAAGGCATTCCTACGGATCTGAGTGGGAAATATCTGAGATTCACAG |
| | | ACCCACTATTGGAACAGTACCAAACCTAGTTTGGCCGATCCATGATT |
| | | ATGTAATGCATATAGTTTTTGTCGATGCTCACCCGTTTCGAGTCTGT |
| | | CTCGTATCGTCTTACGTATAAGTTCAAGCATGTTTACCAGGTCTGTT |
| | | AGAAACTCCTTTGTGAGGGCAGGACCTATTCGTCTCGGTCCCGTTGT |
| | | TTCTAAGAGACTGTACAGCCAAGCGCAGAATGGTGGCATTAACCAT |
| | | AAGAGGATTCTGATCGGACTTGGTCTATTGGCTATTGGAACCACCCT |
| | | TTACGGGACAACCAACCCTACCAAGACTCCTATTGCATTTGTGGAA |
| | | CCAGCCACGGAAAGAGCGTTTAAGGACGGAGACGTCTCTGTGATTT |
| | | TTGTTCTCGGAGGTCCAGGAGCTGGAAAAGGTACCCAATGTGCCAA |
| | | ACTAGTGAGTAATTACGGATTTGTTCACCTGTCAGCTGGAGACTTGT |
| | | TACGTGCAGAACAGAAGAGGGAGGGGTCTAAGTATGGAGAGATGA |
| | | TTTCCCAGTATATCAGAGATGGACTGATAGTACCTCAAGAGGTCAC |
| | | CATTGCGCTCTTGGAGCAGGCCATGAAGGAAAACTTCGAGAAAGGG |
| | | AAGACACGGTTCTTGATTGATGGATTCCCTCGTAAGATGGACCAGG |
| | | CCAAAACTTTTGAGGAAAAAGTCGCAAAGTCCAAGGTGACACTTTT |
| | | CTTTGATTGTCCCGAATCAGTGCTCCTTGAGAGATTACTTAAAAGAG |
| | | GACAGACAAGCGGAAGAGAGGATGATAATGCGGAGAGTATCAAAA |
| | | AAAGATTCAAAACATTCGTGGAAACTTCGATGCCTGTGGTGGACTA |
| | | TTTCGGGAAGCAAGGACGCGTTTTGAAGGTATCTTGTGACCACCCT |
| | | GTGGATCAAGTGTATTCACAGGTTGTGTCGGTGCTAAAAGAGAAGG |
| | | GGATCTTTGCCGATAACGAGACGGAGAATAAATAA |
| 257 | PpRPL10 promoter | GTTCTTCGCTTGGTCTTGTATCTCCTTACACTGTATCTTCCCATTTGC |
| | | GTTTAGGTGGTTATCAAAAACTAAAAGGAAAAATTTCAGATGTTTA |
| | | TCTCTAAGGTTTTTTCTTTTTACAGTATAACACGTGATGCGTCACGT |
| | | GGTACTAGATTACGTAAGTTATTTTGGTCCGGTGGGTAAGTGGGTA |
| | | AGAATAGAAAGCATGAAGGTTTACAAAAACGCAGTCACGAATTATT |
| | | GCTACTTCGAGCTTGGAACCACCCCAAAGATTATATTGTACTGATGC |
| | | ACTACCTTCTCGATTTTGCTCCTCCAAGAACCTACGAAAAACATTTC |
| | | TTGAGCCTTTTCAACCTAGACTACACATCAAGTTATTTAAGGTATGT |
| | | TCCGTTAACATGTAAGAAAAGGAGAGGATAGATCGTTTATGGGGTA |
| | | CGTCGCCTGATTCAAGCGTGACCATTCGAAGAATAGGCCTTCGAAA |
| | | GCTGAATAAAGCAAATGTCAGTTGCGATTGGTATGCTGACAAATTA |
| | | GCATAAAAAGCAATAGACTTTCTAACCACCTGTTTTTTTCCTTTTAC |
| | | TTTATTTATATTTTGCCACCGTACTAACAAGTTCAGACAAA |
| 306 | Sequence of the 5′- | CCATAGCCTCTGATTGATGTAAGCACCGACAGTACCTGGCTCTAACT |
| | Region used for | TGTTAGAGGTTTTGGTGGTCAAGACATATCTGTTATCACAAATAACA |
| | knock out of YOS9 | TAATGGTTATCGGGAAAGTCATTGGGATGAACAGCAAGTGTGTTCA |
| | | TGATGGCAAATTCATTACCCGGAGAGTTGACTATCTTCAATACATGC |
| | | ACCTTTGGAGCATTTCTCTTTGTGAATCCCAGTTTTTCCATGGTTGTG |
| | | GCAAAGTGTAGAGATGTTAAGTGCAGCGAGCAAAGACAAGTAGAT |
| | | AGACTGTATGGTGTTCTGATGTTATAGTTGTAGTGAATAATCTATAA |
| | | ATGCCTTATTTGAAGGTTTATGTAATAGATTTACCCGTGTGTAGCAA |
| | | GTGTACTGCTAAGAGGTACTATAAAGTTATTCATGTGGATATATTCA |
| | | GTAGATAATAACAAAGCTACAAGGAGATCAAGAAACCATATGAGT |
| | | TGTTCGTCACATAAGAGATTACGTAATGACAAATCGGGGAACTAGT |
| | | ACCAATTCTGTCTTAAAGTAGTGTCTCTCTAAGCATAACGACCTATT |
| | | TGATAACTGGGCTGAACTCCAAGCAGCCTGATGATGTTGACCTGAC |
| | | TTATTCAGAAGGGCTATTGGTTTTGATTTCCAGATATTAGCATAATT |
| | | AGCAATGCCGGAACAATATACATCCAATATTTTTGAATGAATGAAC |
| | | GGTTATCAACATTTACTTCTGCCTCCTCGTCTATGACTTCCTTGAGTT |
| | | CCAGCTTGTTATCGGATCTGATTTTTTTGATTTTCTTTTCTTTTCTTGG |
| | | TAGTTTGGGAATTGGTGCCTGTCGAATTTGTTCAACTATTAGGTTAA |
| | | GACCTTTCTGACTAGCATCGAAGAAGGCTACATTTTCGATGTCGTTG |
| | | TGTTTGTTGATAGTCAGCTTGATATCCTGTGCAATTGGAGAACTTAG |
| | | TCTTTTGTAATTGAAGCAGCCTTCGTCCAAACATATTCTGTAAAGAT |
| | | CACTTGGCAGGTCTAGTTGTTCACCGGTGTGCAATTTCCATTTTGAG |
| | | TCAAATTCTA |
| | | GTGTGGCCAAGTTGAACGAGTTCTGAGCGAAATCAATAGCCTTCAA |
| | | CTGATACGCAAATGTAGACCCCAAGAAAAGAAACAACGTGACGAG |
| | | GCTTTGTAGGGTAGTAGCCATTGTCGAATAGTTGAGGATAAGTAGA |
| | | CGGCGAGTTATTCTCCTTGATAAATGCTATCGCGATGGATAGTGATT |
| | | ACAGTGCGATAATATTATCCTTTTCATCCACGTCAACCATGGTTAAC |
| | | AGGCCATTGGACATTATGATAAAGGTCCTGCTATTCCTGCTCTCCCT |
| | | ATCAAGTCTTGTGAAAGCTTTGGATGATTCCATTGATAAGAATTCTG |
| | | TGGTAAGTCTTTTAATTTTTGTTTTCACAAGATCATGCCGTGCTAACT |
| | | GGGTACTATAGTATACC |
| 307 | Sequence of the 3′- | GGTTCCTATTCACTGAAGACAGAATACCTCATGACACTCCAAACTTT |
| | Region used for | AGAGTGTATAACGGAGTTAATGTGAATTAAGACAATTTATATACTC |
| | knock out of YOS9 | AGTAAAATAAATACTAGTACTTACGTCTTTTTTTAGTCAGAGCACTA |
| | | ACTCTGCTGGAAGGGTTCTTCGTGTAAATTGGTACAGACGCTGGTA |
| | | AAGTACCACTATACGTTGTTTGACAAATAGGTAGTTTGAAGCTGAC |
| | | ATCAAGTTTCAAGTCCTTAGGAGTCACATTGCGAGTTTGAATGACC |
| | | AATTGTATTAATCTCTTAATCTTGAAGTACAATCTCTTCTCTTTGAGA |
| | | CTGGGTTTCAAGACAGTGACGGGATTAGCAGGATCGATTTTGGGTG |
| | | ATGCCTTATACCTTTCTTGACGTAATTGTGACAGATCTATTAGCAAC |
| | | TTGCTTATAAGTTCTTGCTCTTTGTTGGAACGGATAGCCTCTATCTCA |
| | | TCCTCCTCAACGAAGCTTCCCGGAGTCCAGGAGAGGAGGTTGTCTA |
| | | GCTTGATCTTATAGTCTTCGGATCCATTGACCTGGACTTCCTTATCTG |
| | | TGTTTTCAAGTTTAGTTGATGTATCTGTCCCCGTATGGCCATTCTTAG |
| | | TCTCCTGGTCAACAGGTGCCGGAAGCTCTTTTTCAATTCTTTTTGGTT |
| | | CGTCCTTCTGAAGTTCATTATCCGTCTCATTTTTAGATGGTCTGCTCA |
| | | GTTTTTCTGCTATATCACCAAGCTTTCTAAAACCAGCTTGCTCCAGC |
| | | CACCTCAGGCCCTTCAATTCACTGGAGATTGCAGATTTTTCTTCGTC |
| | | TATTGTAGGTGCAAAACTGAAATCGTTACCCTTATTGTGGGTGAGCC |
| | | ATTGACCCATCGGTAACGCGTACCAGTTCAAATGAAAGAGGTTTGG |
| | | CAATAAATCCGTAGGTTTGGTGGCTGGGTGAGGTTCATTGTTGTATT |
| | | GAGGAGAAATCTTGTTAAGCGGCTGTGAACTAATGGAAGGGACATG |
| | | GGGGATTACTTTCGTCAGATTAAAATCGCCTTCATTCACTACAGCTT |
| | | CTCTAGCATCCAAGCTTGATTTATTATTCAGGGACGAAAACAATGG |
| | | CGCATTAGGTGTGATGAATGTAGTTAAACATTCTCCGTTGGATGAA |
| | | ACAAAAAATGTGGACACTTTATTGAAGTCTTTTGTCATCGATTCTTC |
| | | AAACTCACTGGTGTAATCATCTAAAACACGAGAGTCAACGCTTTCT |
| | | CTTAGTTGTCTGTAGTTGAACAAAAATCTTCCTGCCTCTCTGATCAA |
| | | TAACTCAACCATCGACTTGTAGAACAAATCAATCTTGACGTAGTCTT |
| | | CCGAATCTCTGTTCCGTTCGTTTATAAGTATCAGGCACACTAAAGTT |
| | | AGGTCGTGAAATATGGAATAAATAGTCTTGTAGTGACCACTCTTTAT |
| | | TCTGTCGCTGATGGTAACCAGCTCTGTAGGTTTGAGATCCTTACCAT |
| | | CAACAAGCTGATAGTATGATCCAGCTATCAAGGAAGGATCCTGGAC |
| 308 | Sequence of the 5′- | AACCTTCATGGAACGATTCGGATACGGAAAAACCTGAGATAGTTTT |
| | Region used for | AACTAGAGTAGATGCAAGATTTCACGATTCTAAAGACCGAGAAGGA |
| | knock out of ALG3 | GATGTCTGATGTCGGTAACTACTATCCGGTAAATGATATTAGCACA |
| | | CTATATGCTACTAGCGAGTCTGGAACCAATTCTACTATCCATTGATG |
| | | CTCTATTAGGGATGGAGAATTCAATCAACCCCTCTAATTCTGATTTC |
| | | AGATGTTCCAACAGCGAAGTAGCCCTTGACAAGTTCTCAACATCAC |
| | | TCATCTTAGCTACATTCACGTATGCTTTGATAAAAAACTCTCTACTT |
| | | TTGTCAATGAGCTCTAGCCTAGTCTCTGGTTCTATCGTTTCCTCTTTG |
| | | GTCTCCAGATTACTCTCTGGATTAGAATCTACATCCATCTTCATATC |
| | | TATGTCCATGTCCAGCTCAATTTTCATACCGTCAGTATTCTTAGATTC |
| | | GATAGCAGTATCTGATCTGGTAGATCCATTAGTTGCTGCAGCGGTAT |
| | | TTTCTTTGGAATTTGGAGCACTTTCCTGTTTCTGTTTCATAAAGACTC |
| | | GGTAGATTGCAATGACTATATCGTTTCTGTAGAACTTGTAACCATGA |
| | | GTCCAAAATTGGGTTTCAGGCATGTATCCTAGCTCATCTAAATATCC |
| | | AACCACATCATCCGTGCTACATATAGTAGACTCGTAGAGTGTCTGT |
| | | GAAGAAACGGCTCTTTTTCCTGCCAAAGGAACGTCCGATATTTGAA |
| | | GGGTCCATATACGATTTTCCTTATTAAGAGCTTCAAGATGTTTCTTA |
| | | TTAAACAATTCAAAGTCTTTTAATTCAATTGTGTTATCAATAGGATC |
| | | CTCAACGTCCTGTTTCCATTCGGTGGACATTCTCATCTTGTATTGTTC |
| | | GATTTGGTTGACTTTTCCAGTCTGGAACTCAGGACTATAAGGAAACT |
| | | TTGGAGTTAAAATAACAGTATAAGTTGAGAGCCTTGCGGGCACCAT |
| | | ACCCGTTAGAGACTTCAACGTCTCCAAGATCAACTGCAGTTGAGAC |
| | | TCTTGGATTCTAGATACCAGAGACACCTGTTGTACCATATAATTAAG |
| | | TGACTGGGCTGGCTTGGATACAGGATTTCGAGAAGTGCTTCGAATT |
| | | ATCAGACCGAAGGCAGTTGATATTTTGTGCCTCAGCCTTAATGTTCC |
| | | CTATAACTTAAGGCTATACACAGCTTTATGATTAATGAATCTGGGCT |
| | | GCTGGTGACGAATTTCGTCAATGACCAGTTGCCTACGGGCGATAAT |
| | | TATTTTTTCAGTTGGATGAAAGAACGGAAAAACCCGGTCAGATTCA |
| | | AAAAGAATATTGATAATCTTTGTCTAGCACAACTGAAATGCTTGGA |
| | | AACTCTCCCAAGCATGAATCAGACCTGAGATTGTATTAGACGAAAA |
| | | AATTGTAGTATAGAGTTATAGACATATAGGTTGTGGCAATATCCTGT |
| | | GCAAGCCAATATCTCACAGAAATAAACGTACACACCAGATACAACT |
| | | ATTTCGAAAAGCACACTTTGAGCGCAACAGTGATTGTCCTAACAGT |
| | | ATAGGTTTCTAAGGCCCCAGCAGACCATGACGGCAAATTATTTATTT |
| | | CCCCTCGTATTTGCCTTATCTCCTTTTGTTCTCATTCTTATCTTGGCTA |
| | | CTGTAATTATCTGGATAACCCTCGATACTTCGCTTGGTTTCTACCTC |
| | | ACAACATATCCCTACC |
| 309 | Sequence of the 3′- | ATTTACAATTAGTAATATTAAGGTGGTAAAAACATTCGTAGAATTG |
| | Region used for | AAATGAATTAATATAGTATGACAATGGTTCATGTCTATAAATCTCCG |
| | knock out of ALG3 | GCTTCGGTACCTTCTCCCCAATTGAATACATTGTCAAAATGAATGGT |
| | | TGAACTATTAGGTTCGCCAGTTTCGTTATTAAGAAAACTGTTAAAAT |
| | | CAAATTCCATATCATCGGTTCCAGTGGGAGGACCAGTTCCATCGCC |
| | | AAAATCCTGTAAGAATCCATTGTCAGAACCTGTAAAGTCAGTTTGA |
| | | GATGAAATTTTTCCGGTCTTTGTTGACTTGGAAGCTTCGTTAAGGTT |
| | | AGGTGAAACAGTTTGATCAACCAGCGGCTCCCGTTTTCGTCGCTTAG |
| | | TAGCAGCATTATTACCAGGAATGCCGCCTGTAGAGTTTTGATGTGTC |
| | | CTAGCTGCAATTGGAGTCTGTGGAGTAGTGGGAGTCGGGGGCTCAG |
| | | TAGCTTTCTTTGCCTTCTTTTTAGCTGGCTCCTTTTTCTTTCGTACAG |
| | | GTGCGACATTATTTGGTGTAGACCCCGCAGAAGTGTTACCAGTACT |
| | | ATGTGCAGTGTTTTGAGTTTGTGTACCAGGTGAAGTTCCGGGAGTAT |
| | | TCTTCGTGACCACTGCAGAGTTCTGGGGAGGGAGCATTACATTCAC |
| | | ATTAAATTTTGGTTCGGGCGGTGTGTGCTCTGGAATTGGATCAAAGT |
| | | TAGAAAAATGCCCGCTTCCCTTCTTACATGCCATGTCATGACGCTGT |
| | | TTGTTCTGTTTCTCAAGCATCATTAGCTCTTTCTGATACTCCTGTATA |
| | | CCTACAATTTTAGAAGCACTTGATTGAGACTGTTGCGATTGCTGGTG |
| | | TTGGCTCTGTGATTGTGGTTGTGCTATTTGCTGATGTTGTGACCCTG |
| | | GAGTTGGAACTAGCTCCGGCTGCTGAATAGAAGAAGGCGGAGAAT |
| | | GTTGCGGTTGAGATGCAGGTAAAGGCTGCTGATAAACAGGACCAGG |
| | | TTGCGAGAATCTAGGTGTGGTGGACGAGTGAGGAGTACCGGCGGCA |
| | | GAAGTAGAGTGAGGCAGAGGAGCCAT |
| 310 | LmSTT3A (DNA) | ATGCCAGCTAAGAACCAACATAAGGGTGGTGGTGATGGTGATCCAG |
| | | ACCCAACTTCTACTCCAGCTGCTGAGTCCACTAAGGTTACAAACACT |
| | | TCCGATGGTGCTGCTGTTGATTCTACTTTGCCACCATCCGACGAGAC |
| | | TTACTTGTTCCACTGTAGAGCTGCTCCATACTCCAAGTTGTCCTACG |
| | | CTTTCAAGGGTATCATGACTGTTTTGATCTTGTGTGCTATCAGATCC |
| | | GCTTACCAAGTTAGATTGATCTCCGTTCAAATCTACGGTTACTTGAT |
| | | CCACGAATTTGACCCATGGTTCAACTACAGAGCTGCTGAGTACATG |
| | | TCTACTCACGGTTGGTCTGCTTTTTTCTCCTGGTTCGATTACATGTCC |
| | | TGGTATCCATTGGGTAGACCAGTTGGTTCTACTACTTACCCAGGATT |
| | | GCAGTTGACTGCTGTTGCTATCCATAGAGCTTTGGCTGCTGCTGGAA |
| | | TGCCAATGTCCTTGAACAATGTTTGTGTTTTGATGCCAGCTTGGTTT |
| | | GGTGCTATCGCTACTGCTACTTTGGCTTTGATCGCTTTCGAAGTTTCC |
| | | GAGTCCATTTGTATGGCTGCTTGGGCTGCTTTGTCCTTCTCCATTATC |
| | | CCTGCTCACTTGATGAGATCCATGGCTGGTGAGTTCGACAACGAGT |
| | | GTATTGCTGTTGCTGCTATGTTGTTGACTTTCTACTGTTGGGTTAGAT |
| | | CCTTGAGAACTAGATCCTCCTGGCCAATCGGTGTTTTGACTGGTGTT |
| | | GCTTACGGTTACATGGCTGCTGCTTGGGGAGGTTACATCTTCGTTTT |
| | | GAACATGGTTGCTATGCACGCTGGTATCTCTTCTATGGTTGACTGGG |
| | | CTAGAAACACTTACAACCCATCCTTGTTGAGAGCTTACACTTTGTTC |
| | | TACGTTGTTGGTACTGCTATCGCTGTTTGTGTTCCACCAGTTGGAAT |
| | | GTCTCCATTCAAGTCCTTGGAGCAGTTGGGAGCTTTGTTGGTTTTGG |
| | | TTTTCTTGTGTGGATTGCAAGTTTGTGAGGTTTTGAGAGCTAGAGCT |
| | | GGTGTTGAAGTTAGATCCAGAGCTAATTTCAAGATCAGAGTTAGAG |
| | | TTTTCTCCGTTATGGCTGGTGTTGCTGCTTTGGCTATCTCTGTTTTGG |
| | | CTCCAACTGGTTACTTTGGTCCATTGTCTGTTAGAGTTAGAGCTTTG |
| | | TTCGTTGAGCACACTAGAACTGGTAACCCATTGGTTGACTCCGTTGC |
| | | TGAACATCATCCAGCTGACGCTTTGGCTTACTTGAACTACTTGCACA |
| | | TCGTTCACTTGATGTGGATCTGTTCCTTGCCAGTTCAGTTGATCTTGC |
| | | CATCCAGAAACCAGTACGCTGTTTTGTTCGTTTTGGTCTACT |
| | | CCTTCATGGCTTACTACTTCTCCACTAGAATGGTTAGATTGTTGATC |
| | | TTGGCTGGTCCAGTTGCTTGTTTGGGAGCTTCTGAAGTTGGTGGTAC |
| | | TTTGATGGAATGGTGTTTCCAGCAATTGTTCTGGGACAACGGAATG |
| | | AGAACTGCTGATATGGTTGCTGCTGGTGACATGCCATACCAAAAGG |
| | | ACGATCACACTTCCAGAGGTGCTGGTGCTAGACAAAAGCAGCAGAA |
| | | GCAAAAGC |
| | | CAGGTCAAGTTTCTGCTAGAGGATCTTCTACTTCCTCCGAGGAAAG |
| | | ACCATACAGAACTTTGATCCCAGTTGACTTCAGAAGAGATGCTCAG |
| | | ATGAACAGATGGTCCGCTGGTAAAACTAACGCTGCTTTGATCGTTG |
| | | CTTTGACTATCGGAGTTTTGTTGCCATTGGCTTTCGTTTTCCACTTGT |
| | | CCTGTATCTCTTCCGCTTACTCTTTTGCTGGTCCAAGAATCGTTTTCC |
| | | AGACTCAGTTGCACACTGGTGAACAGGTTATCGTTAAGGACTACTT |
| | | GGAAGCTTACGAGTGGTTGAGAGACTCTACTCCAGAGGACGCTAGA |
| | | GTTTTGGCTTGGTGGGACTACGGTTACCAAATCACTGGTATCGGTAA |
| | | CAGAACTTCCTTGGCTGATGGTAACACTTGGAACCACGAGCACATT |
| | | GCTACTATCGGAAAGATGTTGACTTCTCCAGTTGCTGAAGCTCACTC |
| | | CTTGGTTAGACACATGGCTGACTACGTTTTGATTTGGGCTGGTCAAT |
| | | CTGGTGACTTGATGAAGTCTCCACACATGGCTAGAATCGGTAACTC |
| | | TGTTTACCACGACATTTGTCCAGATGACCCATTGTGTCAGCAATTCG |
| | | GTTTCCACAGAAACGATTACTCCAGACCAACTCCAATGATGAGAGC |
| | | TTCCTTGTTGTACAACTTGCACGAGGCTGGAAAGACTAAGGGTGTT |
| | | AAGGTTAACCCATCTTTGTTCCAAGAGGTTTACTCCTCCAAGTACGG |
| | | TTTGGTTAGAATCTTCAAGGTTATGAACGTTTCCGCTGAGTCTAAGA |
| | | AGTGGGTTGCAGACCCAGCTAACAGAGTTTGTCACCCACCTGGTTCT |
| | | TGGATTTGTCCTGGTCAATACCCACCTGCTAAAGAAATCCAAGAGA |
| | | TGTTGGCTCACAGAGTTCCATTCGACCAAATGGACAAGCACAAGCA |
| | | GCACAAAGAAACTCACCACAAGGCATAA |
| 311 | LmSTT3B (DNA) | ATGTTGTTGTTGTTCTTCTCCTTCTTGTACTGTTTGAAGAACGCTTAC |
| | | GGATTGAGAATGATCTCCGTTCAAATCTACGGTTACTTGATCCACGA |
| | | ATTTGACCCATGGTTCAACTACAGAGCTGCTGAGTACATGTCTACTC |
| | | ACGGTTGGTCTGCTTTTTTCTCCTGGTTCGATTACATGTCCTGGTATC |
| | | CATTGGGTAGACCAGTTGGTTCTACTACTTACCCAGGATTGCAGTTG |
| | | ACTGCTGTTGCTATCCATAGAGCTTTGGCTGCTGCTGGAATGCCAAT |
| | | GTCCTTGAACAATGTTTGTGTTTTGATGCCAGCTTGGTTTGGTGCTA |
| | | TCGCTACTGCTACTTTGGCTTTGATGACTTACGAAATGTCCGGTTCC |
| | | GGTATTGCTGCTGCTATTGCTGCTTTCATCTTCTCCATCATCCCAGCT |
| | | CATTTGATGAGATCCATGGCTGGTGAGTTCGACAACGAGTGTATTG |
| | | CTGTTGCTGCTATGTTGTTGACTTTCTACTGTTGGGTTAGATCCTTGA |
| | | GAACTAGATCCTCCTGGCCAATCGGTGTTTTGACTGGTGTTGCTTAC |
| | | GGTTACATGGCAGCTGCTTGGGGAGGTTACATCTTCGTTTTGAACAT |
| | | GGTTGCTATGCACGCTGGTATCTCTTCTATGGTTGACTGGGCTAGAA |
| | | ACACTTACAACCCATCCTTGTTGAGAGCTTACACTTTGTTCTACGTT |
| | | GTTGGTACTGCTATCGCTGTTTGTGTTCCACCAGTTGGAATGTCTCC |
| | | ATTCAAGTCCTTGGAGCAGTTGGGAGCTTTGTTGGTTTTGGTTTTCT |
| | | TGTGTGGATTGCAAGTTTGTGAGGTTTTGAGAGCTAGAGCTGGTGTT |
| | | GAAGTTAGATCCAGAGCTAATTTCAAGATCAGAGTTAGAGTTTTCT |
| | | CCGTTATGGCTGGTGTTGCTGCTTTGGCTATCTCTGTTTTGGCTCCAA |
| | | CTGGTTACTTTGGTCCATTGTCTGTTAGAGTTAGAGCTTTGTTCGTTG |
| | | AGCACACTAGAACTGGTAACCCATTGGTTGACTCCGTTGCTGAACA |
| | | CAGAATGACTTCCCCAAAGGCTTACGCTTTCTTCTTGGACTTCACTT |
| | | ACCCAGTTTGGTTGTTGGGTACTGTTTTGCAGTTGTTGGGAGCATTC |
| | | ATGGGTTCCAGAAAAGAGGCTAGATTGTTCATGGGATTGCATTCCT |
| | | TGGCTACTTACTACTTCGCTGATAGAATGTCCAGATTGATCGTTTTG |
| | | GCTGGTCCAGCTGCTGCTGCTATGACTGCTGGAATCTTGGGATTGGT |
| | | TTACGAATGGTGTTGGGCTCAATTGACTGGATGGGCTTCTCCTGGTT |
| | | TGTCTGCTGCTGGTTCTGGTGGAATGGATGACTTCGACAACAAGAG |
| | | AGGACAAACTCAAATCCAGTCCTCCACTGCTAATAGAAACAGAGGT |
| | | GTTAGAGCACATGCTATCGCTGCTGTTAAGTCCATTAAGGCTGGTGT |
| | | TAACTTGTTGCCATTGGTTTTGAGAGTTGGTGTTGCTGTTGCTATTTT |
| | | GGCTGTTACTGTTGGTACTCCATACGTTTCCCAGTTCCAGGCTAGAT |
| | | GTATTCAATCCGCTTACTCCTTTGCTGGTCCAAGAATCGTTTTCCAG |
| | | GCTCAGTTGCACACTGGTGAACAGGTTATCGTTAAGGACTACTTGG |
| | | AAGCTTACGAGTGGTTGAGAGACTCTACTCCAGAGGACGCTAGAGT |
| | | TTTGGCTTGGTGGGACTACGGTTACCAAATCACTGGTATCGGTAAC |
| | | AGAACTTCCTTGGCTGATGGTAACACTTGGAACCACGAGCACATTG |
| | | CTACTATCGGAAAGATGTTGACTTCTCCAGTTGCTGAAGCTCACTCC |
| | | TTGGTTAGACACATGGCTGACTACGTTTTGATTTGGGCTGGTCAATC |
| | | TGGTGACTTGATGAAGTCTCCACACATGGCTAGAATCGGTAACTCT |
| | | GTTTACCACGACATTTGTCCAGATGACCCATTGTGTCAGCAATTCGG |
| | | TTTCCACAGAAACGATTACTCCAGACCAACTCCAATGATGAGAGCT |
| | | TCCTTGTTGTACAACTTGCACGAGGCTGGTAAAACTAAGGGTGTTA |
| | | AGGTTAACCCATCTTTGTTCCAAGAGGTTTACTCCTCCAAGTACGGT |
| | | TTGGTTAGAATCTTCAAGGTTATGAACGTTTCCGCTGAGTCTAAGAA |
| | | GTGGGTTGCAGACCCAGCTAACAGAGTTTGTCACCCACCTGGTTCTT |
| | | GGATTTGTCCTGGTCAATACCCACCTGCTAAAGAAATCCAAGAGAT |
| | | GTTGGCTCACAGAGTTCCATTCGACCAAATGGACAAGCACAAGCAG |
| | | CACAAAGAAACTCACCACAAGGCATAA |
| 312 | Pichia pastoris ATT1 | GGCCGGGACTACATGAGGCCGATTCTTCAAGCCAGGGAAATTAATT |
| | 5′ region in | GCTTGAACCGGAAAATCATTAAGGCAGGCAACGAAAAATCCAACTC |
| | pGLY5933 | CTTGGTTGAATTGACTCAAAAGTTTATCTTACGGAGAAAAGCTAAA |
| | | GACATCAATACGAATTTCCTTCCGCCAAAAACTGAACTGATACTGA |
| | | TGGTTCCAATGACTGAATTACAACAGGAGCTATACAAGGATATAAT |
| | | TGAAACTAACCAAGCCAAGCTTGGCTTGATCAACGACAGAAACTTT |
| | | TTTCTTCAAAAAATTTTGATTCTTCGTAAAATATGCAATTCACCCTC |
| | | CCTGCTGAAAGACGAACCTGATTTTGCCAGATACAATCTCGGCAAT |
| | | AGATTCAATAGCGGTAAGATCAAGCTAACAGTACTGCTTTTACGAA |
| | | AGCTGTTTGAAACCACCAATGAGAAGTGTGTGATTGTTTCAAACTTC |
| | | ACTAAAACTTTGGACGTACTTCAGCTAATCATAGAGCACAACAATT |
| | | GGAAATACCACCGACTAGATGGTTCGAGTAAAGGACGGGACAAAA |
| | | TCGTACGAGATTTTAACGAGTCGCCTCAAAAAGATCGATTCATCAT |
| | | GTTGCTTTCTTCCAAGGCAGGGGGAGTGGGGCTCAACTTAATTGGA |
| | | GCCTCACGCTTAATTCTTTTTGATAACGACTGGAATCCCAGTGTTGA |
| | | CATTCAAGCAATGGCTAGAGTGCATCGAGACGGGCAGAAAAGGCA |
| | | CACCTTTATCTATCGTTTGTATACGAAAGGCACAATTGACGAAAAG |
| | | ATCCTACAAAGGCAATTGATGAAACAAAATCTGAGCGACAAATTCC |
| | | TGGATGATAATGATAGCAGCAAGGATGATGTGTTTAACGACTACGA |
| | | TCTCAAAGATTTGTTTACTGTAGATCTTGACACGAATTGTAGTACAC |
| | | ACGATTTGATGGAATGTTTATGTAATGGGCGGCTGAGAGATCCGAC |
| | | TCCCGTCTTGGAAGCAGAAGAATGCAAGACAAAACCGTTGGAGGCC |
| | | GTTGACGACACGGATGATGGTTGGATGTCAGCTCTGGATTTCAAAC |
| | | AGTTATCACAAAAAGAGGAGACAGGTGCTGTGTCAACAATGCGTCA |
| | | ATGTCTGCTCGGATATCAACACATTGATCCAAAGATTTTGGAACCA |
| | | ACAGAACCTGTAGGGGACGATTTGGTATTGGCAAACATCCTCGCGG |
| | | AGTCCTCAGGCTTGGCTAAATCTGCATTGTCATCTGAAAAGAAACC |
| | | CAAGAAACCAGTGGTGAACTTTATCTTTGTGTCAGGCCAAGACTAA |
| | | GCTGGAAGAACGGAACTTTAATCGAAGGAAAAATTAAATGTCAAA |
| | | GTGGGTCGATCAGGAGATAATCCATGCTTCACGTGATTTTTCTTAAT |
| | | AAACGCCGGAAAAACTTTCTTTTTTGTGACCAAAATTATCCGATCTG |
| | | AAAAAAAATTACGCATGCGTGAAGTAGGATGAGAGACTTACTGTTG |
| | | AACTTTGTGAGACGAGGGGAAAAGGAATATCCTGATCGTAAACAA |
| | | AAAAGTTTTCCAGCCCAATCGGGAACATCTGCGAAGTGTTGGAATT |
| | | CAACCCCTCTTTCGAAAATGTTCCATTTTACCCAAAATTATTGTTATT |
| | | AAATAATACATGTGTTACTAGCAAAGTCTGCGCTTTCCATGTCTCAG |
| | | ATTCGGCAGATAACAAAGTTGACACGTTCTTGCGAGATACGCATGA |
| | | ATCTTTTGGCTGCTTTTTGTGAAAGAGAAATGGTGCCATATATTGCA |
| | | GACGCCCCTGAAAGATTAGTGTGCGGCTGAGTCTTTTTTTTTTCTCA |
| | | ACCAGCTTTTTCTTTTTATTGGGTACCATCGCGCACGCAGGACTCAT |
| | | GCTCCATTAGACTTCTGAACCACCTGACTTAATATTCATGGACGGAC |
| | | GCTTTTATCCTTAAATTGTTCATCCATTCCTCAATTTTTCCGTTTGCC |
| | | CTCCCTGTACTATTAAATTACAAAAGCTGATCTTTTTCAAGTGTTTCT |
| | | CTTTGAATCGCTC |
| 313 | Pichia pastoris ATT1 | GGACCCTGAAGACGAAGACATGTCTGCCTTAGAGTTTACCGCAGTT |
| | 3′ region in | CGATTCCCCAACTTTTCAGCTACGACAACAGCCCCGCCTCCTACTCC |
| | pGLY5933: | AGTCAATTGCAACAGTCCTGAAAACATCAAGACCTCCACTGTGGAC |
| | | GATTTTTTGAAAGCTACTCAAGATCCAAATAACAAAGAGATACTCA |
| | | ACGACATTTACAGTTTGATTTTTGATGACTCCATGGATCCTATGAGC |
| | | TTCGGAAGTATGGAACCAAGAAACGATTTGGAAGTTCCGGACACTA |
| | | TAATGGATTAATTTGCAGCGGGCCTGTTTGTATAGTCTTTGATTGTG |
| | | TATAATAGAATTACTACGCGTATATCCCGATCTGGAAGTAACATGG |
| | | AAGTTTCCCATTTTCGCGCAGTCTCCTACTCGTATCCTCCCCACCCCT |
| | | TACCGATGACGCAAAAGGTCACTAGATAAGCATAGCATAGTTTCAT |
| | | CCCTTGCTCTTTCCTTGTACCAACAGATCATGGCTGGGAATCTCAAG |
| | | GATATTCTATCCTTGTCGAGGAAGACAGCAAGGAATCTGAAGCAGG |
| | | CTCTGGATGAGCTTGCGGAGCAGGTGATCAACCACCAACGGAGACG |
| | | ACCAGCTCTGGTCCGAGTTCCTATCAACAACAACCTTAGGCGCAAG |
| | | AGCCAGCAGTCCTTTTTGAATCGCAGGTCATTCCATCTTTGGACCAG |
| | | CAAGTACAACCCATACTTTTGGAGGGGAGGCAGAAGCAACGTTCTG |
| | | GACCAGCTTAACCGTGAAGCTTTAAGGTACAGATCGTCTTTTGCGA |
| | | AACCCGGATTTTATCCAAGTGGGCTGTATCAGTCAACTTTCCCTCAA |
| | | AGAGGTAGTAGGATGTTTTCCACCTGCGCCTACTCATGTCAGCAGG |
| | | AGGCAGTCAAAAACTTGACTTCCGCTGTTCGTGCTTTGTTACAAAGT |
| | | GGTGCTAATTTCGGCAGTCAAATGAAACAAATGAAACACTGTTCGC |
| | | AAAAGAAGAAGCACTTCTCTAAATTTTCTAAGAGGCTTACTTCTTCC |
| | | ACTGCCGCTGGGTCTGGCAAGAATGCTGAACAAGCTCCTTCTGGTTT |
| | | GGCCGAAGGATCCGCTGTTGTTTTTAGCCTTGAACGTCAAAGTCAC |
| | | AATACTGAGTTGGAAGGAATCTTGGATCAAGAAACTTCTTCCATTCT |
| | | CGAGGAAGAAATGGTTCAACATGAGCGTCACCTGGCTATTATTAGA |
| | | GAAGAAATCCAGAGAATTAGTGAGAATCTAGGATCATTACCATTAA |
| | | TCATGTCTGGTCACAAGATTGAGGTATTTTTCCCCAATTGTGACACT |
| | | GTTAAATGTGAGCAACTGATGAGAGATTTGGCTATTACGAAAGGGG |
| | | TTGTGAGGCGTCATGATTCTACTGCTGAGCATTCAAGCTCCAGGTCA |
| | | TTTGTTCCAGAAGATTGCTTGTATTCCTCAGGGTCAAGTTCACCGAA |
| | | TCCTTTATCCTCAACTTCTTCGAAATCATTTGATAGAGTCTCATTGG |
| | | ACTACATTTCCTCTCGGTCTACATCTGATCAAACCACTGGTTCTGAG |
| | | TACACATCTCTGTCTCAACAATATCACCTGGTTAGCAATTACAACCC |
| | | TGTACTATCCTCAGCCCCGGGTTCTTCGAGGGTCTTGGAGCTGAATA |
| | | CTCCCGAGTCCACTATGGAAGGCAGTACAGATCTGGAGTATTTAAC |
| | | GCGAGACGATGTGTTGCTGTTAAATGTCTAATCTAGACCTATCCTTC |
| | | ATTCTATATAGCTTAGTTGAGTTTTACGTAAGCCCTAGTTTTTGTTA |
| | | ATTCTTATCGATTTATGGTTAGTGTACCACTCAACTCACGATGATAT |
| | | ATCCCAGGAGCTGTTTGTGCATTATAACTACCAATCCT |
| 314 | DNA encodes Mus | ATGGCTAAGTTTAGAAGAAGAACCTGTATTTTGTTGTCCTTGTTTAT |
| | muscula | CCTTTTTATTTTCTCCTTGATGATGGGATTGAAGATGCTTTGGCCTAA |
| | endomannosidase | CGCTGCCTCTTTTGGTCCACCTTTCGGATTGGATTTGCTTCCAGAACT |
| | (codon-optimized for | TCATCCTTTGAACGCACACTCAGGTAATAAGGCTGATTTTCAGAGA |
| | expression in Pichia | AGTGACAGAATTAACATGGAAACTAACACAAAGGCTTTGAAAGGT |
| | pastoris) | GCCGGAATGACTGTTCTTCCTGCCAAAGCATCCGAGGTCAACCTTG |
| | | AAGAGTTGCCACCTCTTAACTACTTTTTGCATGCTTTCTACTACTCAT |
| | | GGTACGGTAACCCACAATTCGATGGAAAGTACATCCATTGGAATCA |
| | | CCCAGTTTTGGAACATTGGGACCCTAGAATCGCTAAAAATTACCCA |
| | | CAGGGTCAACACTCTCCACCTGATGACATTGGTTCTTCCTTCTACCC |
| | | TGAATTGGGATCTTATTCAAGTAGAGATCCATCCGTTATTGAGACTC |
| | | ATATGAAGCAAATGAGATCCGCCTCCATCGGTGTCTTGGCACTTTCA |
| | | TGGTACCCACCTGACAGTAGAGATGACAACGGAGAAGCCACAGAT |
| | | CACTTGGTTCCTACCATTCTTGACAAGGCACATAAGTACAACTTGAA |
| | | GGTCACTTTCCACATCGAGCCATATTCTAATAGAGATGACCAGAAC |
| | | ATGCACCAAAACATCAAGTACATCATCGATAAGTACGGTAACCATC |
| | | CTGCTTTCTACAGATATAAGACCAGAACTGGACACTCTTTGCCAATG |
| | | TTCTACGTTTATGACTCCTACATTACAAAACCTACCATCTGGGCTAA |
| | | CTTGCTTACTCCATCAGGTAGTCAGTCGGTTAGATCCTCCCCTTATG |
| | | ATGGATTGTTTATTGCCTTGCTTGTCGAAGAGAAGCATAAGAACGA |
| | | TATCTTGCAGTCTGGTTTCGACGGAATCTACACATATTTTGCTACCA |
| | | ACGGTTTCACTTACGGATCAAGTCACCAAAATTGGAACAATTTGAA |
| | | GTCCTTCTGTGAAAAGAACAATCTTATGTTCATCCCATCAGTTGGTC |
| | | CTGGATATATTGATACAAGTATCAGACCATGGAACACTCAAAACAC |
| | | AAGAAACAGAGTTAACGGTAAATACTACGAGGTCGGATTGTCTGCA |
| | | GCTCTTCAGACTCATCCTTCCTTGATTTCAATCACAAGTTTTAACGA |
| | | ATGGCACGAGGGTACTCAAATTGAAAAGGCTGTTCCAAAAAGAACC |
| | | GCCAATACTATCTACTTGGATTATAGACCACATAAGCCTTCATTGTA |
| | | CCTTGAGTTGACCAGAAAATGGTCTGAAAAGTTCTCCAAAGAGAGA |
| | | ATGACTTATGCATTGGACCAACAGCAACCAGCTTCCTAA |
| 315 | Pichia pastoris | TCAAGAGGATGTCAGAATGCCATTTGCCTGAGAGATGCAGGCTTCA |
| | AOX1 transcription | TTTTGATACTTTTTTATTTGTAACCTATATAGTATAGGATTTTTTTTG |
| | termination | TCATTTTGTTTCTTCTCGTACGAGCTTGCTCCTGATCAGCCTATCTCG |
| | sequences | CAGCTGATGAATATCTTGTGGTAGGGGTTTGGGAAAATCATTCGAG |
| | | TTTGATGTTTTTCTTGGTATTTCCCACTCCTCTTCAGAGTACAGAAGA |
| | | TTAAGTGAGACGTTCGTTTGTGCA |

While the present invention is described herein with reference to illustrated embodiments, it should be understood that the invention is not limited hereto. Those having ordinary skill in the art and access to the teachings herein will recognize additional modifications and embodiments within the scope thereof. Therefore, the present invention is limited only by the claims attached herein.

1. A composition comprising:
a glycosylated insulin or insulin analogue having an A-chain peptide comprising the amino acid sequence GIVEQCCTSICSLYQLENYCN (SEQ ID NO: 33); and a B-chain peptide comprising the amino acid sequence HLCGSHLVEALYLVCGERGFF (SEQ ID NO:161),
wherein at least one amino acid residue of the A-chain peptide or B-chain peptide amino acid sequence is covalently linked to an N-glycan; and
wherein the insulin or insulin analogue optionally further includes up to 17 amino acid substitutions and/or a polypeptide of 3 to 35 amino acids covalently linked to the N-terminus of the A-chain peptide or B-chain peptide, the C-terminus of the A-chain peptide or B-chain peptide, at the N-terminus to the C-terminus of the B-chain peptide and at the C-terminus to the N-terminus of the A-chain peptide, or combinations thereof; and
a pharmaceutically acceptable carrier.

2. The composition of claim 1, wherein the N-glycan is covalently linked to the amide group of an Asn residue in a β1 linkage.

3. The composition of claim 2, wherein the Asn residue is at amino acid position 10 or 21 of the native A-chain peptide or amino acid position 3, 25, or 28 of the native B-chain peptide with the proviso that if the Asn is at the 3 position of the B-chain then the amino acid at position 5 of the B-chain peptide is a Ser or Thr and if the Asn is at position 21 of the A-chain then the A-chain peptide further includes at the C-terminus of the Asn a dipeptide of amino acid sequence Xaa-Ser or Xaa-Thr wherein Xaa is any amino acid except Pro.

4. The composition of claim 1, wherein a tripeptide having the amino acid sequence Asn-Xaa-Ser or Asn-Xaa-Thr wherein Xaa is any amino acid except Pro is covalently linked to the N-terminus of the A-chain or the N-terminus or C-terminus of the B-chain in a peptide bond.

5. The composition of claim 1, wherein the N-glycan is attached to the insulin or insulin molecule at a histidine, cysteine, or lysine residue.

6. The composition of claim 1, wherein the insulin or insulin analogue is a heterodimer or a single-chain.

7. The composition of claim 1, wherein the B-chain peptide lacks a threonine residue at position 30.

8. The composition of claim 1, wherein the N-glycan is a paucimannose, high mannose, hybrid, or complex glycan.

9. The composition of claim 1, wherein the N-glycan consists of a Man₃GlcNAc₂ glycan structure or a fucosylated Man₃GlcNAc₂ structure; a Man₅GlcNAc₂, Man₆GlcNAc₂, Man₇GlcNAc₂, Man₈GlcNAc₂, or Man₉GlcNAc₂ structure; a GlcNAcMan₃GlcNAc₂; GalGlcNAcMan₃GlcNAc₂; NANAGalGlcNAcMan₃GlcNAc₂; GlcNAcMan₅GlcNAc₂; GalGlcNAcMan₅GlcNAc₂; or NANAGalGlcNAcMan₅GlcNAc₂ structure; a fucosylated or non-fucosylated GlcNAc₂Man₃GlcNAc₂; GalGlcNAc₂Man₃GlcNAc₂; Gal₂GlcNAc₂Man₃GlcNAc₂; NANAGal₂GlcNAc₂Man₃GlcNAc₂; or NANA₂Gal₂GlcNAc₂Man₃GlcNAc₂ structure; or a fucosylated or non-fucosylated glycan having a structure selected from the group consisting of Man₃GlcNAc₂; Man₅GlcNAc₂; GlcNAc₍₁₋₄₎Man₃GlcNAc₂; Gal₍₁₋₄₎GlcNAc₍₁₋₄₎Man₃GlcNAc₂; and NANA₍₁₋₄₎Gal₍₁₋₄₎GlcNAc₍₁₋₄₎Man₃GlcNAc₂ structures.

10. The composition of claim 1, wherein at least 70%, 75%, 80%, 85%, 90%, 95%, 96%, 97%, 98%, 99%, or 100% of the insulin or insulin analogues include the N-glycan.

11. A pharmaceutical formulation comprising:
(a) a multiplicity of glycosylated insulin or insulin analogues, each glycosylated insulin or insulin analogue having at least one N-glycan thereon, wherein the predominant N-glycan consists of a high mannose, hybrid, complex, or paucimannose N-glycan, and
(b) a pharmaceutically acceptable carrier.

12. The pharmaceutical formulation of claim 11, wherein the N-glycan consists of a Man₃GlcNAc₂ N-glycan structure or a fucosylated Man₃GlcNAc₂ N-glycan structure; a Man₅GlcNAc₂, Man₆GlcNAc₂, Man₇GlcNAc₂, Man₈GlcNAc₂, or Man₉GlcNAc₂ structure; a GlcNAcMan₃GlcNAc₂; GalGlcNAcMan₃GlcNAc₂; NANAGalGlcNAcMan₃GlcNAc₂; GlcNAcMan₅GlcNAc₂; GalGlcNAcMan₅GlcNAc₂; or NANAGalGlcNAcMan₅GlcNAc₂ structure; a fucosylated or non-fucosylated GlcNAc₂Man₃GlcNAc₂; GalGlcNAc₂Man₃GlcNAc₂; Gal₂GlcNAc₂Man₃GlcNAc₂; NANAGal₂GlcNAc₂Man₃GlcNAc₂; or NANA₂Gal₂GlcNAc₂Man₃GlcNAc₂ structure; or a fucosylated or non-fucosylated glycan having a structure selected from the group consisting of Man₃GlcNAc₂; Man₅GlcNAc₂; GlcNAc₍₁₋₄₎Man₃GlcNAc₂; Gal₍₁₋₄₎GlcNAc₍₁₋₄₎Man₃GlcNAc₂; and NANA₍₁₋₄₎Gal₍₁₋₄₎GlcNAc₍₁₋₄₎Man₃GlcNAc₂ structures.

13. The pharmaceutical formulation of claim 11, wherein at least 70%, 75%, 80%, 85%, 90%, 95%, 96%, 97%, 98%, 99%, or 100% of the insulin or insulin analogues are N-glycosylated.

14-16. (canceled)

17. A method for altering a pharmacokinetic or pharmacodynamic property of an insulin or insulin analogue, comprising:
attaching an N-glycan to an amino acid residue of the insulin or insulin analogue to produce a glycosylated insulin or insulin analogue, wherein the pharmacokinetic property of the glycosylated insulin or insulin analogue that is attached to the N-glycan is altered compared to the insulin or insulin analogue not attached to the N-glycan.

18. The method of claim 17, wherein the N-glycan is attached to the amino acid residue in vitro.

19. The method of claim 17, wherein the N-glycan is attached to the amino acid residue in vivo by
(a) providing a host cell capable of producing glycoproteins;
(b) introducing into the host cell a nucleic acid molecule encoding an insulin or insulin analogue comprising an N-linked glycosylation site;
(c) cultivating the host cell in a medium and under conditions to produce a glycosylated proinsulin or proinsulin analogue precursor or the glycosylated insulin analogue; and
(d) recovering the glycosylated proinsulin or proinsulin analogue precursor from the medium and processing the glycosylated proinsulin or proinsulin analogue precursor in vitro to produce the glycosylated insulin or insulin analogue or recovering glycosylated insulin analogue from the medium to produce the glycosylated insulin or insulin analogue.

20-22. (canceled)

23. A method for producing an insulin or insulin analogue that has at least one pharmacokinetic or pharmacodynamic property sensitive to serum concentration of glucose when used in a treatment for diabetes, comprising:
attaching an N-glycan to an amino acid residue of the insulin or insulin analogue to produce a glycosylated insulin or insulin analogue, wherein the glycosylated insulin or insulin analogue that is attached to the N-glycan has at least one pharmacokinetic or pharmacodynamic property of the insulin or insulin analogue that is attached to the N-glycan is sensitive to serum concentration of glucose.

24. The method of claim 23, wherein the N-glycan is attached to the amino acid residue in vitro.

25. The method of claim 23, wherein the N-glycan is attached to the amino acid residue in vivo by
(a) providing a host cell capable of producing glycoproteins;
(b) introducing into the host cell a nucleic acid molecule encoding an insulin or insulin analogue comprising an N-linked glycosylation site;
(c) cultivating the host cell in a medium and under conditions to produce a glycosylated proinsulin or proinsulin analogue precursor or the glycosylated insulin analogue; and
(d) recovering the glycosylated proinsulin or proinsulin analogue precursor from the medium and processing the glycosylated proinsulin or proinsulin analogue precursor in vitro to produce the glycosylated insulin or insulin analogue or recovering glycosylated insulin analogue from the medium to produce the glycosylated insulin or insulin analogue.

26.-27. (canceled)

28. A glycosylated insulin or insulin analogue having an A-chain peptide comprising the amino acid sequence GIVEQCCTSICSLYQLENYCN (SEQ ID NO: 33); and a B-chain peptide comprising the amino acid sequence HLCGSHLVEALYLVCGERGFF (SEQ ID NO:161),
wherein at least one amino acid residue of the A-chain peptide or B-chain peptide amino acid sequence is covalently linked to an N-glycan; and
wherein the insulin or insulin analogue optionally further includes up to 17 amino acid substitutions and/or a polypeptide of 3 to 35 amino acids covalently linked to N-terminus, C-terminus, or which is covalently linked at the N-terminus to the C-terminus of the B-chain and at the C-terminus to the N-terminus of the A-chain; and
a pharmaceutically acceptable carrier for the treatment of diabetes.

29. (canceled)


SEQUENCE LISTING (337 sequences)

SEQ ID NO: 1 - DNA, 29 bp, Artificial Sequence
  MAM508
catcattattagcttactttcataattgc

SEQ ID NO: 2 - DNA, 23 bp, Artificial Sequence
  MAM509
catgcgtacacgcgtttgtacag

SEQ ID NO: 3 - DNA, 45 bp, Artificial Sequence
  MAM564
gcaaaaggccggccttattaaccgcagtagttctccaattggtac

SEQ ID NO: 4 - DNA, 85 bp, Artificial Sequence
  MAM864
aaaagagtcctcttgaagaaggtcaccaccatcaccatcatcaccatcatcacgaaccaaagtttgttaatcaacacttgtgtgg

SEQ ID NO: 5 - DNA, 378 bp, Artificial Sequence
  DNA encoding pre-proinsulin analogue Yps1ss+TA57 propeptide+N-terminal spacer+B chain P28N+C-peptide “AAK”+ insulin A chain
atgaagttgaagactgttagatccgctgttttgtcttctttgtttgcttctcaagttttgggtcaaccaattgatgatactgaatctcaaactacttctgttaacttgatggctgatgatactgaatctgcttttgctactcaaactaactctggtggtttggatgttgttggtttgatttctatggctaagagagaagaaggtgaaccaaagtttgttaaccaacatttgtgtggttctcatttggttgaagctttgtacttggtttgtggtgaaagaggttttttttacactaacaagactgctgctaagggtattgttgaacaatgttgtacttctatttgttctttgtaccaattggaaaactactgtaactaa

SEQ ID NO: 6 - protein, 125 aa, Artificial Sequence
  Pre-proinsulin analogue Yps1ss+TA57 propeptide+N-terminal spacer+B chain P28N+C-peptide “AAK”+ insulin A chain
MKLKTVRSAVLSSLFASQVLGQPIDDTESQTTSVNLMADDTESAFATQTNSGGLDVVGLISMAKREEGEPKFVNQHLCGSHLVEALYLVCGERGFFYTNKTAAKGIVEQCCTSICSLYQLENYCN

SEQ ID NO: 7 - DNA, 408 bp, Artificial Sequence
  DNA encoding pre-proinsulin analogue S.c. alpha mating factor signal sequence and pro-peptide+N-terminal spacer+B chain P28N+C-peptide “A(10xHIS)AK”+ insulin A chain
atgaagttgaagactgttagatccgctgttttgtcttctttgtttgcttctcaagttttgggtcaaccaattgatgatactgaatctcaaactacttctgttaacttgatggctgatgatactgaatctgcttttgctactcaaactaactctggtggtttggatgttgttggtttgatttctatggctaagagagaagaaggtgaaccaaagtttgttaaccaacatttgtgtggttctcatttggttgaagctttgtacttggtttgtggtgaaagaggttttttttacactaacaagactgctcaccaccatcaccatcatcaccatcatcacgctaagggtattgttgaacaatgttgtacttctatttgttctttgtaccaattggaaaactactgtaactaa

SEQ ID NO: 8 - protein, 135 aa, Artificial Sequence
  Pre-proinsulin analogue Yps1ss+TA57 propeptide+N-terminal spacer+B chain P28N+C-peptide “A(10xHIS)AK”+ insulin A chain
MKLKTVRSAVLSSLFASQVLGQPIDDTESQTTSVNLMADDTESAFATQTNSGGLDVVGLISMAKREEGEPKFVNQHLCGSHLVEALYLVCGERGFFYTNKTAHHHHHHHHHHAKGIVEQCCTSICSLYQLENYCN

SEQ ID NO: 9 - DNA, 417 bp, Artificial Sequence
  DNA encoding pre-proinsulin analogue S.c. alpha mating factor signal sequence and pro-peptide+B chain P28N+C-peptide “RR”+ A chain
atgagatttccttcaatttttactgcagttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctctcgagaaaaggtttgttaatcaacacttgtgtggttcccacttggttgaggctttgtacttggtttgtggtgagagaggtttcttctacactaacaagactagaagaggtatcgttgagcagtgttgtacttccatctgttccttgtaccagttggagaactactgtaactaa

SEQ ID NO: 10 - protein, 138 aa, Artificial Sequence
  Pre-proinsulin analogue S.c. alpha mating factor signal sequence and pro-peptide+B chain P28N+C-peptide “RR”+ A chain
MRFPSIFTAVLFAASSALAAPVNTTTEDETAQIPAEAVIGYLDLEGDFDVAVLPFSNSTNNGLLFINTTIASIAAKEEGVSLEKRFVNQHLCGSHLVEALYLVCGERGFFYTNKTRRGIVEQCCTSICSLYQLENYCN

SEQ ID NO: 11 - DNA, 417 bp, Artificial Sequence
  DNA encoding pre-proinsulin analogue S.c. alpha mating factor signal sequence and pro-peptide+B chain P28N+C-peptide “RR”+ glargine A chain N21G
atgagatttccttcaatttttactgcagttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctctcgagaaaaggtttgttaatcaacacttgtgtggttcccacttggttgaggctttgtacttggtttgtggtgagagaggtttcttctacactaacaagactagaagaggtatcgttgagcagtgttgtacttccatctgttccttgtaccaattggagaactactgcggttaa

SEQ ID NO: 12 - protein, 138 aa, Artificial Sequence
  Pre-proinsulin analogue S.c. alpha mating factor signal sequence and pro-peptide+B chain P28N+C-peptide “RR”+ glargine A chain N21G
MRFPSIFTAVLFAASSALAAPVNTTTEDETAQIPAEAVIGYLDLEGDFDVAVLPFSNSTNNGLLFINTTIASIAAKEEGVSLEKRFVNQHLCGSHLVEALYLVCGERGFFYTNKTRRGIVEQCCTSICSLYQLENYCG

SEQ ID NO: 13 - DNA, 465 bp, Artificial Sequence
  DNA encoding pre-proinsulin analogue S.c. alpha mating factor signal sequence and pro-peptide+N-terminal HIS spacer+B chain P28N+C-peptide “RR”+ glargine A chain N21G
atgagatttccttcaatttttactgcagttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctctcgagaaaagggaagaaggtcaccaccatcaccatcatcaccatcatcacgaaccaaagtttgttaatcaacacttgtgtggttcccacttggttgaggctttgtacttggtttgtggtgagagaggtttcttctacactaacaagactagaagaggtatcgttgagcagtgttgtacttccatctgttccttgtaccaattggagaactactgcggttaa

SEQ ID NO: 14 - protein, 154 aa, Artificial Sequence
  Pre-proinsulin analogue S.c. alpha mating factor signal sequence and pro-peptide+N-terminal HIS spacer+B chain P28N+C-peptide “RR”+ glargine A chain N21G
MRFPSIFTAVLFAASSALAAPVNTTTEDETAQIPAEAVIGYLDLEGDFDVAVLPFSNSTNNGLLFINTTIASIAAKEEGVSLEKREEGHHHHHHHHHHEPKFVNQHLCGSHLVEALYLVCGERGFFYTNKTRRGIVEQCCTSICSLYQLENYCG

SEQ ID NO: 15 - DNA, 495 bp, Artificial Sequence
  DNA encoding pre-proinsulin analogue S.c. alpha mating factor signal sequence and pro-peptide+N-terminal MYC spacer+B chain P28N+ C-peptide “TA(10xHIS)AK”+A chain
atgagattcccatccatcttcactgctgttttgttcgctgcttcctctgctttggctgctccagttaacactactactgaggacgagactgctcagattccagctgaagctgttatcggttacttggacttggagggtgacttcgacgttgctgttttgccattctccaactccactaacaacggtttgttgttcatcaacactactatcgcttccattgctgctaaagaagagggagtttccttggagaagagagaggaacagaagttgatctccgaagaggacttgaacgagaagttcgttaaccagcacttgtgtggttcccacttggttgaggctttgtacttggtttgtggtgagagaggtttcttctacactaacaagactactgctcatcaccatcaccatcatcaccaccatcacgctaagggtatcgttgagcagtgttgtacttccatctgttccttgtaccagttggagaactactgtaactaa

SEQ ID NO: 16 - protein, 164 aa, Artificial Sequence
  Pre-proinsulin analogue S.c. alpha mating factor signal sequence and pro-peptide+N-terminal MYC spacer+B chain P28N+ C-peptide “TA(10xHIS)AK”+A chain
MRFPSIFTAVLFAASSALAAPVNTTTEDETAQIPAEAVIGYLDLEGDFDVAVLPFSNSTNNGLLFINTTIASIAAKEEGVSLEKREEQKLISEEDLNEKFVNQHLCGSHLVEALYLVCGERGFFYTNKTTAHHHHHHHHHHAKGIVEQCCTSICSLYQLENYCN

SEQ ID NO: 17 - DNA, 495 bp, Artificial Sequence
  DNA encoding pre-proinsulin analogue S.c. alpha mating factor signal sequence and pro-peptide+N-terminal MYC spacer+B chain P28N+ C-peptide “TA(10xHIS)AK”+A chain; alternate DNA codon optimization
atgagatttccatctatttttactgctgttttgtttgctgcttcttctgctttggctgctccagttaacactactactgaagatgaaactgctcaaattccagctgaagctgttattggttacttggatttggaaggtgattttgatgttgctgttttgccattttctaactctactaacaacggtttgttgtttattaacactactattgcttctattgctgctaaggaagaaggtgtttctttggaaaagagagaagaacaaaagttgatttctgaagaagatttgaacgaaaagtttgttaaccaacatttgtgtggttctcatttggttgaagctttgtacttggtttgtggtgaaagaggttttttttacactaacaagactactgctcatcatcatcatcatcatcatcatcatcatgctaagggtattgttgaacaatgttgtacttctatttgttctttgtaccaattggaaaactactgtaactaa

SEQ ID NO: 18 - protein, 163 aa, Artificial Sequence
  Pre-proinsulin analogue S.c. alpha mating factor signal sequence and pro-peptide+N-terminal MYC spacer+B chain P28N+ C-peptide “TA(10xHIS)AK”+A chain; alternate DNA codon optimization
MRFPSIFTAVLFAASSALAAPVNTTTEDETAQIPAEAVIGYLDLEGDFDVAVLPFSNSTNNGLLFINTTIASIAAKEEGVSLEKREEQKLISEEDLNEKFVNQHLCGSHLVALYLVCGERGFFYTNKTTAHHHHHHHHHHAKGIVEQCCTSICSLYQLENYCN

SEQ ID NO: 19 - protein, 85 aa, Artificial Sequence
  Sc alpha mating factor signal sequence and pro-peptide
MRFPSIFTAVLFAASSALAAPVNTTTEDETAQIPAEAVIGYLDLEGDFDVAVLPFSNSTNNGLLFINTTIASIAAKEEGVSLEKR

SEQ ID NO: 20 - protein, 21 aa, Artificial Sequence
  Yps1ss leader
MKLKTVRSAVLSSLFASQVLG

SEQ ID NO: 21 - protein, 44 aa, Artificial Sequence
  TA57 pro
QPIDDTESQTTSVNLMADDTESAFATQTNSGGLDVVGLISMAKR

SEQ ID NO: 22 - protein, 6 aa, Artificial Sequence
  N-terminal spacer
EEGEPK

SEQ ID NO: 23 - protein, 16 aa, Artificial Sequence
  N-terminal HIS spacer
EEGHHHHHHHHHHEPK

SEQ ID NO: 24 - protein, 14 aa, Artificial Sequence
  N-terminal MYC spacer
EEQKLISEEDLNEK

SEQ ID NO: 25 - protein, 30 aa, Homo sapiens
FVNQHLCGSHLVEALYLVCGERGFFYTPKT

SEQ ID NO: 26 - protein, 30 aa, Artificial Sequence
  Insulin B chain P28N
FVNQHLCGSHLVEALYLVCGERGFFYTNKT

SEQ ID NO: 27 - protein, 32 aa, Artificial Sequence
  Insulin Glargine B chain
FVNQHLCGSHLVEALYLVCGERGFFYTPKTRR

SEQ ID NO: 28 - protein, 53 aa, Artificial Sequence
  Insulin Glargine B chain  P28N proinsulin
FVNQHLCGSHLVEALYLVCGERGFFYTNKTRRGIVEQCCTSICSLYQLENYCG

SEQ ID NO: 29 - protein, 53 aa, Artificial Sequence
  Insulin Glargine B chain  P28N proinsulin with glulisine mutation (B chain N3K)
FVKQHLCGSHLVEALYLVCGERGFFYTNKTRRGIVEQCCTSICSLYQLENYCG

SEQ ID NO: 30 - protein, 35 aa, Homo sapiens
RREAEDLQVGQVELGGGPGAGSLQPLALEGSLQKR

SEQ ID NO: 31 - protein, 3 aa, Artificial Sequence
  C peptide “AAK”
AAK

SEQ ID NO: 32 - protein, 13 aa, Artificial Sequence
  C peptide “HIS”
AHHHHHHHHHHAK

SEQ ID NO: 33 - protein, 21 aa, Homo sapies
GIVEQCCTSICSLYQLENYCN

SEQ ID NO: 34 - protein, 21 aa, Artificial Sequence
  Insulin glargine A chain N21G
GIVEQCCTSICSLYQLENYCG

SEQ ID NO: 35 - protein, 110 aa, Homo sapiens
MALWMRLLPLLALLALWGPDPAAAFVNQHLCGSHLVEALYLVCGERGFFYTPKTRREAEDLQVGQVELGGGPGAGSLQPLALEGSLQKRGIVEQCCTSICSLYQLENYCN

SEQ ID NO: 36 - protein, 60 aa, Artificial Sequence
  B chain P28N proinsulin with N-terminal spacer and C-peptide “AAK”
EEGEPKFVNQHLCGSHLVEALYLVCGERGFFYTNKTAAKGIVEQCCTSICSLYQLENYCN

SEQ ID NO: 37 - protein, 54 aa, Artificial Sequence
  B chain P28N proinsulin with C-peptide “AAK”
FVNQHLCGSHLVEALYLVCGERGFFYTNKTAAKGIVEQCCTSICSLYQLENYCN

SEQ ID NO: 38 - protein, 53 aa, Artificial Sequence
  Proinsulin B (P28N) with C-chain “RR”
FVNQHLCGSHLVEALYLVCGERGFFYTNKTRRGIVEQCCTSICSLYQLENYCN

SEQ ID NO: 39 - protein, 70 aa, Artificial Sequence
  B chain P28N proinsulin with N-terminal spacer and C-peptide “A(10xHIS)AK”
EEGEPKFVNQHLCGSHLVEALYLVCGERGFFYTNKTAHHHHHHHHHHAKGIVEQCCTSICSLYQLENYCN

SEQ ID NO: 40 - protein, 79 aa, Artificial Sequence
  B chain P28N proinsulin with N-terminal spacer (myc epitope) and C-peptide “A(10xHIS)AK”
EEQKLISEEDLNEKFVNQHLCGSHLVEALYLVCGERGFFYTNKTTAHHHHHHHHHHAKGIVEQCCTSICSLYQLENYCN

SEQ ID NO: 41 - protein, 69 aa, Artificial Sequence
  B chain P28N glargine proinsulin with N-terminal HIS spacer
EEGHHHHHHHHHHEPKFVNQHLCGSHLVEALYLVCGERGFFYTNKTRRGIVEQCCTSICSLYQLENYCG

SEQ ID NO: 42 - protein, 30 aa, Artificial Sequence
  B chain H5S
FVNQSLCGSHLVEALYLVCGERGFFYTPKT

SEQ ID NO: 43 - protein, 30 aa, Artificial Sequence
  B chain H5T
FVNQTLCGSHLVEALYLVCGERGFFYTPKT

SEQ ID NO: 44 - protein, 30 aa, Artificial Sequence
  B chain F25N
FVNQHLCGSHLVEALYLVCGERGFNYTPKT

SEQ ID NO: 45 - protein, 21 aa, Artificial Sequence
  A chain I10N
GIVEQCCTSNCSLYQLENYCN

SEQ ID NO: 46 - DNA, 3029 bp, Saccharomycese cerevisiea
aggcctcgcaacaacctataattgagttaagtgcctttccaagctaaaaagtttgaggttataggggcttagcatccacacgtcacaatctcgggtatcgagtatagtatgtagaattacggcaggaggtttcccaatgaacaaaggacaggggcacggtgagctgtcgaaggtatccattttatcatgtttcgtttgtacaagcacgacatactaagacatttaccgtatgggagttgttgtcctagcgtagttctcgctcccccagcaaagctcaaaaaagtacgtcatttagaatagtttgtgagcaaattaccagtcggtatgctacgttagaaaggcccacagtattcttctaccaaaggcgtgcctttgttgaactcgatccattatgagggcttccattattccccgcatttttattactctgaacaggaataaaaagaaaaaacccagtttaggaaattatccgggggcgaagaaatacgcgtagcgttaatcgaccccacgtccagggtttttccatggaggtttctggaaaaactgacgaggaatgtgattataaatccctttatgtgatgtctaagacttttaaggtacgcccgatgtttgcctattaccatcatagagacgtttcttttcgaggaatgcttaaacgactttgtttgacaaaaatgttgcctaagggctctatagtaaaccatttggaagaaagatttgacgactttttttttttggatttcgatcctataatccttcctcctgaaaagaaacatataaatagatatgtattattcttcaaaacattctcttgttcttgtgctttttttttaccatatatcttacttttttttttctctcagagaaacaagcaaaacaaaaagcttttcttttcactaacgtatatgatgcttttgcaagctttccttttccttttggctggttttgcagccaaaatatctgcatcaatgacaaacgaaactagcgatagacctttggtccacttcacacccaacaagggctggatgaatgacccaaatgggttgtggtacgatgaaaaagatgccaaatggcatctgtactttcaatacaacccaaatgacaccgtatggggtacgccattgttttggggccatgctacttccgatgatttgactaattgggaagatcaacccattgctatcgctcccaagcgtaacgattcaggtgctttctctggctccatggtggttgattacaacaacacgagtgggtttttcaatgatactattgatccaagacaaagatgcgttgcgatttggacttataacactcctgaaagtgaagagcaatacattagctattctcttgatggtggttacacttttactgaataccaaaagaaccctgttttagctgccaactccactcaattcagagatccaaaggtgttctggtatgaaccttctcaaaaatggattatgacggctgccaaatcacaagactacaaaattgaaatttactcctctgatgacttgaagtcctggaagctagaatctgcatttgccaatgaaggtttcttaggctaccaatacgaatgtccaggtttgattgaagtcccaactgagcaagatccttccaaatcttattgggtcatgtttatttctatcaacccaggtgcacctgctggcggttccttcaaccaatattttgttggatccttcaatggtactcattttgaagcgtttgacaatcaatctagagtggtagattttggtaaggactactatgccttgcaaactttcttcaacactgacccaacctacggttcagcattaggtattgcctgggcttcaaactgggagtacagtgcctttgtcccaactaacccatggagatcatccatgtctttggtccgcaagttttctttgaacactgaatatcaagctaatccagagactgaattgatcaatttgaaagccgaaccaatattgaacattagtaatgctggtccctggtctcgttttgctactaacacaactctaactaaggccaattcttacaatgtcgatttgagcaactcgactggtaccctagagtttgagttggtttacgctgttaacaccacacaaaccatatccaaatccgtctttgccgacttatcactttggttcaagggtttagaagatcctgaagaatatttgagaatgggttttgaagtcagtgcttcttccttctttttggaccgtggtaactctaaggtcaagtttgtcaaggagaacccatatttcacaaacagaatgtctgtcaacaaccaaccattcaagtctgagaacgacctaagttactataaagtgtacggcctactggatcaaaacatcttggaattgtacttcaacgatggagatgtggtttctacaaatacctacttcatgaccaccggtaacgctctaggatctgtgaacatgaccactggtgtcgataatttgttctacattgacaagttccaagtaagggaagtaaaatagaggttataaaacttattgtcttttttatttttttcaaaagccattctaaagggctttagctaacgagtgacgaatgtaaaactttatgatttcaaagaatacctccaaaccattgaaaatgtatttttatttttattttctcccgaccccagttacctggaatttgttctttatgtactttatataagtataattctcttaaaaatttttactactttgcaatagacatcattttttcacgtaataaacccacaatcgtaatgtagttgccttacactactaggatggacctttttgcctttatctgttttgttactgacacaatgaaaccgggtaaagtattagttatgtgaaaatttaaaagcattaagtagaagtataccatattgtaaaaaaaaaaagcgttgtcttctacgtaaaagtgttctcaaaaagaagtagtgagggaaatggataccaagctatctgtaacaggagctaaaaaatctcagggaaaagcttctggtttgggaaacggtcgac

SEQ ID NO: 47 - DNA, 898 bp, Artificial Sequence
  Sequence of the 5′-Region used for knock out of PpURA5
atcggcctttgttgatgcaagttttacgtggatcatggactaaggagttttatttggaccaagttcatcgtcctagacattacggaaagggttctgctcctctttttggaaactttttggaacctctgagtatgacagcttggtggattgtacccatggtatggcttcctgtgaatttctattttttctacattggattcaccaatcaaaacaaattagtcgccatggctttttggcttttgggtctatttgtttggaccttcttggaatatgctttgcatagatttttgttccacttggactactatcttccagagaatcaaattgcatttaccattcatttcttattgcatgggatacaccactatttaccaatggataaatacagattggtgatgccacctacacttttcattgtactttgctacccaatcaagacgctcgtcttttctgttctaccatattacatggcttgttctggatttgcaggtggattcctgggctatatcatgtatgatgtcactcattacgttctgcatcactccaagctgcctcgttatttccaagagttgaagaaatatcatttggaacatcactacaagaattacgagttaggctttggtgtcacttccaaattctgggacaaagtctttgggacttatctgggtccagacgatgtgtatcaaaagacaaattagagtatttataaagttatgtaagcaaataggggctaatagggaaagaaaaattttggttctttatcagagctggctcgcgcgcagtgtttttcgtgctcctttgtaatagtcatttttgactactgttcagattgaaatcacattgaagatgtcactcgaggggtaccaaaaaaggtttttggatgctgcagtggcttcgc

SEQ ID NO: 48 - DNA, 1060 bp, Artificial Sequence
  Sequence of the 3′-Region used for knock out of PpURA5
ggtcttttcaacaaagctccattagtgagtcagctggctgaatcttatgcacaggccatcattaacagcaacctggagatagacgttgtatttggaccagcttataaaggtattcctttggctgctattaccgtgttgaagttgtacgagctcggcggcaaaaaatacgaaaatgtcggatatgcgttcaatagaaaagaaaagaaagaccacggagaaggtggaagcatcgttggagaaagtctaaagaataaaagagtactgattatcgatgatgtgatgactgcaggtactgctatcaacgaagcatttgctataattggagctgaaggtgggagagttgaaggtagtattattgccctagatagaatggagactacaggagatgactcaaataccagtgctacccaggctgttagtcagagatatggtacccctgtcttgagtatagtgacattggaccatattgtggcccatttgggcgaaactttcacagcagacgagaaatctcaaatggaaacgtatagaaaaaagtatttgcccaaataagtatgaatctgcttcgaatgaatgaattaatccaattatcttctcaccattattttcttctgtttcggagctttgggcacggcggcgggtggtgcgggctcaggttccctttcataaacagatttagtacttggatgcttaatagtgaatggcgaatgcaaaggaacaatttcgttcatctttaaccctttcactcggggtacacgttctggaatgtacccgccctgttgcaactcaggtggaccgggcaattcttgaactttctgtaacgttgttggatgttcaaccagaaattgtcctaccaactgtattagtttccttttggtcttatattgttcatcgagatacttcccactctccttgatagccactctcactcttcctggattaccaaaatcttgaggatgagtcttttcaggctccaggatgcaaggtatatccaagtacctgcaagcatctaatattgtctttgccagggggttctccacaccatactccttttggcgcatgc

SEQ ID NO: 49 - DNA, 957 bp, Artificial Sequence
  Sequence of the PpURA5 auxotrophic marker
tctagagggacttatctgggtccagacgatgtgtatcaaaagacaaattagagtatttataaagttatgtaagcaaataggggctaatagggaaagaaaaattttggttctttatcagagctggctcgcgcgcagtgtttttcgtgctcctttgtaatagtcatttttgactactgttcagattgaaatcacattgaagatgtcactggaggggtaccaaaaaaggtttttggatgctgcagtggcttcgcaggccttgaagtttggaactttcaccttgaaaagtggaagacagtctccatacttctttaacatgggtcttttcaacaaagctccattagtgagtcagctggctgaatcttatgctcaggccatcattaacagcaacctggagatagacgttgtatttggaccagcttataaaggtattcctttggctgctattaccgtgttgaagttgtacgagctgggcggcaaaaaatacgaaaatgtcggatatgcgttcaatagaaaagaaaagaaagaccacggagaaggtggaagcatcgttggagaaagtctaaagaataaaagagtactgattatcgatgatgtgatgactgcaggtactgctatcaacgaagcatttgctataattggagctgaaggtgggagagttgaaggttgtattattgccctagatagaatggagactacaggagatgactcaaataccagtgctacccaggctgttagtcagagatatggtacccctgtcttgagtatagtgacattggaccatattgtggcccatttgggcgaaactttcacagcagacgagaaatctcaaatggaaacgtatagaaaaaagtatttgcccaaataagtatgaatctgcttcgaatgaatgaattaatccaattatcttctcaccattattttcttctgtttcggagctttgggcacggcggcggatcc

SEQ ID NO: 50 - DNA, 709 bp, Artificial Sequence
  Sequence of the part of the Ec lacZ gene that was used to construct the PpURA5 blaster (recyclable auxotrophic marker)
cctgcactggatggtggcgctggatggtaagccgctggcaagcggtgaagtgcctctggatgtcgctccacaaggtaaacagttgattgaactgcctgaactaccgcagccggagagcgccgggcaactctggctcacagtacgcgtagtgcaaccgaacgcgaccgcatggtcagaagccgggcacatcagcgcctggcagcagtggcgtctggcggaaaacctcagtgtgacgctccccgccgcgtcccacgccatcccgcatctgaccaccagcgaaatggatttttgcatcgagctgggtaataagcgttggcaatttaaccgccagtcaggctttctttcacagatgtggattggcgataaaaaacaactgctgacgccgctgcgcgatcagttcacccgtgcaccgctggataacgacattggcgtaagtgaagcgacccgcattgaccctaacgcctgggtcgaacgctggaaggcggcgggccattaccaggccgaagcagcgttgttgcagtgcacggcagatacacttgctgatgcggtgctgattacgaccgctcacgcgtggcagcatcaggggaaaaccttatttatcagccggaaaacctaccggattgatggtagtggtcaaatggcgattaccgttgatgttgaagtggcgagcgatacaccgcatccggcgcggattggcctgaactgccag

SEQ ID NO: 51 - DNA, 2875 bp, Artificial Sequence
  Sequence of the 5′-Region used for knock out of PpOCH1
aaaaccttttttcctattcaaacacaaggcattgcttcaacacgtgtgcgtatccttaacacagatactccatacttctaataatgtgatagacgaatacaaagatgttcactctgtgttgtgtctacaagcatttcttattctgattggggatattctagttacagcactaaacaactggcgatacaaacttaaattaaataatccgaatctagaaaatgaacttttggatggtccgcctgttggttggataaatcaataccgattaaatggattctattccaatgagagagtaatccaagacactctgatgtcaataatcatttgcttgcaacaacaaacccgtcatctaatcaaagggtttgatgaggcttaccttcaattgcagataaactcattgctgtccactgctgtattatgtgagaatatgggtgatgaatctggtcttctccactcagctaacatggctgtttgggcaaaggtggtacaattatacggagatcaggcaatagtgaaattgttgaatatggctactggacgatgcttcaaggatgtacgtctagtaggagccgtgggaagattgctggcagaaccagttggcacgtcgcaacaatccccaagaaatgaaataagtgaaaacgtaacgtcaaagacagcaatggagtcaatattgataacaccactggcagagcggttcgtacgtcgttttggagccgatatgaggctcagcgtgctaacagcacgattgacaagaagactctcgagtgacagtaggttgagtaaagtattcgcttagattcccaaccttcgttttattctttcgtagacaaagaagctgcatgcgaacatagggacaacttttataaatccaattgtcaaaccaacgtaaaaccctctggcaccattttcaacatatatttgtgaagcagtacgcaatatcgataaatactcaccgttgtttgtaacagccccaacttgcatacgccttctaatgacctcaaatggataagccgcagcttgtgctaacataccagcagcaccgcccgcggtcagctgcgcccacacatataaaggcaatctacgatcatgggaggaattagttttgaccgtcaggtcttcaagagttttgaactcttcttcttgaactgtgtaaccttttaaatgacgggatctaaatacgtcatggatgagatcatgtgtgtaaaaactgactccagcatatggaatcattccaaagattgtaggagcgaacccacgataaaagtttcccaaccttgccaaagtgtctaatgctgtgacttgaaatctgggttcctcgttgaagaccctgcgtactatgcccaaaaactttcctccacgagccctattaacttctctatgagtttcaaatgccaaacggacacggattaggtccaatgggtaagtgaaaaacacagagcaaaccccagctaatgagccggccagtaaccgtcttggagctgtttcataagagtcattagggatcaataacgttctaatctgttcataacatacaaattttatggctgcatagggaaaaattctcaacagggtagccgaatgaccctgatatagacctgcgacaccatcatacccatagatctgcctgacagccttaaagagcccgctaaaagacccggaaaaccgagagaactctggattagcagtctgaaaaagaatcttcactctgtctagtggagcaattaatgtcttagcggcacttcctgctactccgccagctactcctgaatagatcacatactgcaaagactgcttgtcgatgaccttggggttatttagcttcaagggcaatttttgggacattttggacacaggagactcagaaacagacacagagcgttctgagtcctggtgctcctgacgtaggcctagaacaggaattattggctttatttgtttgtccatttcataggcttggggtaatagatagatgacagagaaatagagaagacctaatattttttgttcatggcaaatcgcgggttcgcggtcgggtcacacacggagaagtaatgagaagagctggtaatctggggtaaaagggttcaaaagaaggtcgcctggtagggatgcaatacaaggttgtcttggagtttacattgaccagatgatttggctttttctctgttcaattcacatttttcagcgagaatcggattgacggagaaatggcggggtgtggggtggatagatggcagaaatgctcgcaatcaccgcgaaagaaagactttatggaatagaactactgggtggtgtaaggattacatagctagtccaatggagtccgttggaaaggtaagaagaagctaaaaccggctaagtaactagggaagaatgatcagactttgatttgatgaggtctgaaaatactctgctgctttttcagttgctttttccctgcaacctatcattttccttttcataagcctgccttttctgttttcacttatatgagttccgccgagacttccccaaattctctcctggaacattctctatcgctctccttccaagttgcgccccctggcactgcctagtaatattaccacgcgacttatattcagttccacaatttccagtgttcgtagcaaatatcatcagccatggcgaaggcagatggcagtttgctctactataatcctcacaatccacccagaaggtattacttctacatggctatattcgccgtttctgtcatttgcgttttgtacggaccctcacaacaattatcatctccaaaaatagactatgatccattgacgctccgatcacttgatttgaagactttggaagctccttcacagttgagtccaggcaccgtagaagataatcttcg

SEQ ID NO: 52 - DNA, 997 bp, Artificial Sequence
  Sequence of the 3′-Region used for knock out of PpOCH1
aaagctagagtaaaatagatatagcgagattagagaatgaataccttcttctaagcgatcgtccgtcatcatagaatatcatggactgtatagttttttttttgtacatataatgattaaacggtcatccaacatctcgttgacagatctctcagtacgcgaaatccctgactatcaaagcaagaaccgatgaagaaaaaaacaacagtaacccaaacaccacaacaaacactttatcttctcccccccaacaccaatcatcaaagagatgtcggaaccaaacaccaagaagcaaaaactaaccccatataaaaacatcctggtagataatgctggtaacccgctctccttccatattctgggctacttcacgaagtctgaccggtctcagttgatcaacatgatcctcgaaatgggtggcaagatcgttccagacctgcctcctctggtagatggagtgttgtttttgacaggggattacaagtctattgatgaagataccctaaagcaactgggggacgttccaatatacagagactccttcatctaccagtgttttgtgcacaagacatctcttcccattgacactttccgaattgacaagaacgtcgacttggctcaagatttgatcaatagggcccttcaagagtctgtggatcatgtcacttctgccagcacagctgcagctgctgctgttgttgtcgctaccaacggcctgtcttctaaaccagacgctcgtactagcaaaatacagttcactcccgaagaagatcgttttattcttgactttgttaggagaaatcctaaacgaagaaacacacatcaactgtacactgagctcgctcagcacatgaaaaaccatacgaatcattctatccgccacagatttcgtcgtaatctttccgctcaacttgattgggtttatgatatcgatccattgaccaaccaacctcgaaaagatgaaaacgggaactacatcaaggtacaaggccttcca

SEQ ID NO: 53 - DNA, 2159 bp, kluvermyces  lactis
aaacgtaacgcctggcactctattttctcaaacttctgggacggaagagctaaatattgtgttgcttgaacaaacccaaaaaaacaaaaaaatgaacaaactaaaactacacctaaataaaccgtgtgtaaaacgtagtaccatattactagaaaagatcacaagtgtatcacacatgtgcatctcatattacatcttttatccaatccattctctctatcccgtctgttcctgtcagattctttttccataaaaagaagaagaccccgaatctcaccggtacaatgcaaaactgctgaaaaaaaaagaaagttcactggatacgggaacagtgccagtaggcttcaccacatggacaaaacaattgacgataaaataagcaggtgagcttctttttcaagtcacgatccctttatgtctcagaaacaatatatacaagctaaacccttttgaaccagttctctcttcatagttatgttcacataaattgcgggaacaagactccgctggctgtcaggtacacgttgtaacgttttcgtccgcccaattattagcacaacattggcaaaaagaaaaactgctcgttttctctacaggtaaattacaatttttttcagtaattttcgctgaaaaatttaaagggcaggaaaaaaagacgatctcgactttgcatagatgcaagaactgtggtcaaaacttgaaatagtaattttgctgtgcgtgaactaataaatatatatatatatatatatatatatttgtgtattttgtatatgtaattgtgcacgtcttggctattggatataagattttcgcgggttgatgacatagagcgtgtactactgtaatagttgtatattcaaaagctgctgcgtggagaaagactaaaatagataaaaagcacacattttgacttcggtaccgtcaacttagtgggacagtcttttatatttggtgtaagctcatttctggtactattcgaaacagaacagtgttttctgtattaccgtccaatcgtttgtcatgagttttgtattgattttgtcgttagtgttcggaggatgttgttccaatgtgattagtttcgagcacatggtgcaaggcagcaatataaatttgggaaatattgttacattcactcaattcgtgtctgtgacgctaattcagttgcccaatgctttggacttctctcactttccgtttaggttgcgacctagacacattcctcttaagatccatatgttagctgtgtttttgttctttaccagttcagtcgccaataacagtgtgtttaaatttgacatttccgttccgattcatattatcattagattttcaggtaccactttgacgatgataataggttgggctgtttgtaataagaggtactccaaacttcaggtgcaatctgccatcattatgacgcttggtgcgattgtcgcatcattataccgtgacaaagaattttcaatggacagtttaaagttgaatacggattcagtgggtatgacccaaaaatctatgtttggtatctttgttgtgctagtggccactgccttgatgtcattgttgtcgttgctcaacgaatggacgtataacaagtacgggaaacattggaaagaaactttgttctattcgcatttcttggctctaccgttgtttatgttggggtacacaaggctcagagacgaattcagagacctcttaatttcctcagactcaatggatattcctattgttaaattaccaattgctacgaaacttttcatgctaatagcaaataacgtgacccagttcatttgtatcaaaggtgttaacatgctagctagtaacacggatgctttgacactttctgtcgtgcttctagtgcgtaaatttgttagtcttttactcagtgtctacatctacaagaacgtcctatccgtgactgcatacctagggaccatcaccgtgttcctgggagctggtttgtattcatatggttcggtcaaaactgcactgcctcgctgaaacaatccacgtctgtatgatactcgtttcagaatttttttgattttctgccggatatggtttctcatctttacaatcgcattcttaattataccagaacgtaattcaatgatcccagtgactcgtaactcttatatgtcaatttaagc

SEQ ID NO: 54 - DNA, 870 bp, Artificial Sequence
  Sequence of the 5′-Region used for knock out of PpBMT2
ggccgagcgggcctagattttcactacaaatttcaaaactacgcggatttattgtctcagagagcaatttggcatttctgagcgtagcaggaggcttcataagattgtataggaccgtaccaacaaattgccgaggcacaacacggtatgctgtgcacttatgtggctacttccctacaacggaatgaaaccttcctctttccgcttaaacgagaaagtgtgtcgcaattgaatgcaggtgcctgtgcgccttggtgtattgtttttgagggcccaatttatcaggcgccttttttcttggttgttttcccttagcctcaagcaaggttggtctatttcatctccgcttctataccgtgcctgatactgttggatgagaacacgactcaacttcctgctgctctgtattgccagtgttttgtctgtgatttggatcggagtcctccttacttggaatgataataatcttggcggaatctccctaaacggaggcaaggattctgcctatgatgatctgctatcattgggaagcttcaacgacatggaggtcgactcctatgtcaccaacatctacgacaatgctccagtgctaggatgtacggatttgtcttatcatggattgttgaaagtcaccccaaagcatgacttagcttgcgatttggagttcataagagctcagattttggacattgacgtttactccgccataaaagacttagaagataaagccttgactgtaaaacaaaaggttgaaaaacactggtttacgttttatggtagttcagtctttctgcccgaacacgatgtgcattacctggttagacgagtcatcttttcggctgaaggaaaggcgaactctccagtaacatc

SEQ ID NO: 55 - DNA, 1733 bp, Artificial Sequence
  Sequence of the 3′-Region used for knock out of PpBMT2
ccatatgatgggtgtttgctcactcgtatggatcaaaattccatggtttcttctgtacaacttgtacacttatttggacttttctaacggtttttctggtgatttgagaagtccttattttggtgttcgcagcttatccgtgattgaaccatcagaaatactgcagctcgttatctagtttcagaatgtgttgtagaatacaatcaattctgagtctagtttgggtgggtcttggcgacgggaccgttatatgcatctatgcagtgttaaggtacatagaatgaaaatgtaggggttaatcgaaagcatcgttaatttcagtagaacgtagttctattccctacccaaataatttgccaagaatgcttcgtatccacatacgcagtggacgtagcaaatttcactttggactgtgacctcaagtcgttatcttctacttggacattgatggtcattacgtaatccacaaagaattggatagcctctcgttttatctagtgcacagcctaatagcacttaagtaagagcaatggacaaatttgcatagacattgagctagatacgtaactcagatcttgttcactcatggtgtactcgaagtactgctggaaccgttacctcttatcatttcgctactggctcgtgaaactactggatgaaaaaaaaaaaagagctgaaagcgagatcatcccattttgtcatcatacaaattcacgcttgcagttttgcttcgttaacaagacaagatgtctttatcaaagacccgttttttcttcttgaagaatacttccctgttgagcacatgcaaaccatatttatctcagatttcactcaacttgggtgcttccaagagaagtaaaattcttcccactgcatcaacttccaagaaacccgtagaccagtttctcttcagccaaaagaagttgctcgccgatcaccgcggtaacagaggagtcagaaggtttcacacccttccatcccgatttcaaagtcaaagtgctgcgttgaaccaaggttttcaggttgccaaagcccagtctgcaaaaactagttccaaatggcctattaattcccataaaagtgttggctacgtatgtatcggtacctccattctggtatttgctattgttgtcgttggtgggttgactagactgaccgaatccggtctttccataacggagtggaaacctatcactggttcggttcccccactgactgaggaagactggaagttggaatttgaaaaatacaaacaaagccctgagtttcaggaactaaattctcacataacattggaagagttcaagtttatattttccatggaatggggacatagattgttgggaagggtcatcggcctgtcgtttgttcttcccacgttttacttcattgcccgtcgaaagtgttccaaagatgttgcattgaaactgcttgcaatatgctctatgataggattccaaggtttcatcggctggtggatggtgtattccggattggacaaacagcaattggctgaacgtaactccaaaccaactgtgtctccatatcgcttaactacccatcttggaactgcatttgttatttactgttacatgatttacacagggcttcaagttttgaagaactataagatcatgaaacagcctgaagcgtatgttcaaattttcaagcaaattgcgtctccaaaattgaaaactttcaagagactctcttcagttctattaggcctggtg

SEQ ID NO: 56 - DNA, 981 bp, Mus musculus
  misc_feature (1)..(981): Sequence of the 3′-Region used for knock out of PpBMT2
atgtctgccaacctaaaatatctttccttgggaattttggtgtttcagactaccagtctggttctaacgatgcggtattctaggactttaaaagaggaggggcctcgttatctgtcttctacagcagtggttgtggctgaatttttgaagataatggcctgcatctttttagtctacaaagacagtaagtgtagtgtgagagcactgaatagagtactgcatgatgaaattcttaataagcccatggaaaccctgaagctcgctatcccgtcagggatatatactcttcagaacaacttactctatgtggcactgtcaaacctagatgcagccacttaccaggttacatatcagttgaaaatacttacaacagcattattttctgtgtctatgcttggtaaaaaattaggtgtgtaccagtggctctccctagtaattctgatggcaggagttgcttttgtacagtggccttcagattctcaagagctgaactctaaggacctttcaacaggctcacagtttgtaggcctcatggcagttctcacagcctgtttttcaagtggctttgctggagtttattttgagaaaatcttaaaagaaacaaaacagtcagtatggataaggaacattcaacttggtttctttggaagtatatttggattaatgggtgtatacgtttatgatggagaattggtctcaaagaatggattttttcagggatataatcaactgacgtggatagttgttgctctgcaggcacttggaggccttgtaatagctgctgtcatcaaatatgcagataacattttaaaaggatttgcgacctccttatccataatattgtcaacaataatatcttatttttggttgcaagattttgtgccaaccagtgtctttttccttggagccatccttgtaatagcagctactttcttgtatggttacgatcccaaacctgcaggaaatcccactaaagcatag

SEQ ID NO: 57 - DNA, 486 bp, Artificial Sequence
  PpGAPDH promoter
tttttgtagaaatgtcttggtgtcctcgtccaatcaggtagccatctctgaaatatctggctccgttgcaactccgaacgacctgctggcaacgtaaaattctccggggtaaaacttaaatgtggagtaatggaaccagaaacgtctcttcccttctctctccttccaccgcccgttaccgtccctaggaaattttactctgctggagagcttcttctacggcccccttgcagcaatgctcttcccagcattacgttgcgggtaaaacggaggtcgtgtacccgacctagcagcccagggatggaaaagtcccggccgtcgctggcaataatagcgggcggacgcatgtcatgagattattggaaaccaccagaatcgaatataaaaggcgaacacctttcccaattttggtttctcctgacccaaagactttaaatttaatttatttgtccctatttcaatcaattgaacaactatcaaaacaca

SEQ ID NO: 58 - DNA, 293 bp, Artificial Sequence
  S. cerevisiea CYC transcription termination sequence (ScCYC TT)
acaggccccttttcctttgtcgatatcatgtaattagttatgtcacgcttacattcacgccctcctcccacatccgctctaaccgaaaaggaaggagttagacaacctgaagtctaggtccctatttattttttttaatagttatgttagtattaagaacgttatttatatttcaaatttttcttttttttctgtacaaacgcgtgtacgcatgtaacattatactgaaaaccttgcttgagaaggttttgggacgctcgaaggctttaatttgcaagctgccggctcttaag

SEQ ID NO: 59 - DNA, 1128 bp, Artificial Sequence
  Sequence of the 5′-Region used for knock out of PpMNN4L1
gatctggccattgtgaaacttgacactaaagacaaaactcttagagtttccaatcacttaggagacgatgtttcctacaacgagtacgatccctcattgatcatgagcaatttgtatgtgaaaaaagtcatcgaccttgacaccttggataaaagggctggaggaggtggaaccacctgtgcaggcggtctgaaagtgttcaagtacggatctactaccaaatatacatctggtaacctgaacggcgtcaggttagtatactggaacgaaggaaagttgcaaagctccaaatttgtggttcgatcctctaattactctcaaaagcttggaggaaacagcaacgccgaatcaattgacaacaatggtgtgggttttgcctcagctggagactcaggcgcatggattctttccaagctacaagatgttagggagtaccagtcattcactgaaaagctaggtgaagctacgatgagcattttcgatttccacggtcttaaacaggagacttctactacagggcttggggtagttggtatgattcattcttacgacggtgagttcaaacagtttggtttgttcactccaatgacatctattctacaaagacttcaacgagtgaccaatgtagaatggtgtgtagcgggttgcgaagatggggatgtggacactgaaggagaacacgaattgagtgatttggaacaactgcatatgcatagtgattccgactagtcaggcaagagagagccctcaaatttacctctctgcccctcctcactccttttggtacgcataattgcagtataaagaacttgctgccagccagtaatcttatttcatacgcagttctatatagcacataatcttgcttgtatgtatgaaatttaccgcgttttagttgaaattgtttatgttgtgtgccttgcatgaaatctctcgttagccctatccttacatttaactggtctcaaaacctctaccaattccattgctgtacaacaatatgaggcggcattactgtagggttggaaaaaaattgtcattccagctagagatcacacgacttcatcacgcttattgctcctcattgctaaatcatttactcttgacttcgacccagaaaagttcgcc

SEQ ID NO: 60 - DNA, 1231 bp, Artificial Sequence
  Sequence of the 3′-Region used for knock out of PpMNN4L1
gcatgtcaaacttgaacacaacgactagatagttgttttttctatataaaacgaaacgttatcatctttaataatcattgaggtttacccttatagttccgtattttcgtttccaaacttagtaatcttttggaaatatcatcaaagctggtgccaatcttcttgtttgaagtttcaaactgctccaccaagctacttagagactgttctaggtctgaagcaacttcgaacacagagacagctgccgccgattgttcttttttgtgtttttcttctggaagaggggcatcatcttgtatgtccaatgcccgtatcctttctgagttgtccgacacattgtccttcgaagagtttcctgacattgggcttcttctatccgtgtattaattttgggttaagttcctcgtttgcatagcagtggatacctcgatttttttggctcctatttacctgacataatattctactataatccaacttggacgcgtcatctatgataactaggctctcctttgttcaaaggggacgtcttcataatccactggcacgaagtaagtctgcaacgaggcggcttttgcaacagaacgatagtgtcgtttcgtacttggactatgctaaacaaaaggatctgtcaaacatttcaaccgtgtttcaaggcactctttacgaattatcgaccaagaccttcctagacgaacatttcaacatatccaggctactgcttcaaggtggtgcaaatgataaaggtatagatattagatgtgtttgggacctaaaacagttcttgcctgaagattcccttgagcaacaggcttcaatagccaagttagagaagcagtaccaaatcggtaacaaaagggggaagcatataaaacctttactattgcgacaaaatccatccttgaaagtaaagctgtttgttcaatgtaaagcatacgaaacgaaggaggtagatcctaagatggttagagaacttaacgggacatactccagctgcatcccatattacgatcgctggaagacttttttcatgtacgtatcgcccaccaacctttcaaagcaagctaggtatgattttgacagttctcacaatccattggttttcatgcaacttgaaaaaacccaactcaaacttcatggggatccatacaatgtaaatcattacgagagggcgaggttgaaaagtttccattgcaatcacgtcgcatcatggctactgaaaggccttaac

SEQ ID NO: 61 - DNA, 937 bp, Artificial Sequence
  Sequence of the 5′-Region used for knock out of PpPNO1 and PpMNN4
tcattctatatgttcaagaaaagggtagtgaaaggaaagaaaaggcatataggcgagggagagttagctagcatacaagataatgaaggatcaatagcggtagttaaagtgcacaagaaaagagcacctgttgaggctgatgataaagctccaattacattgccacagagaaacacagtaacagaaataggaggggatgcaccacgagaagagcattcagtgaacaactttgccaaattcataaccccaagcgctaataagccaatgtcaaagtcggctactaacattaatagtacaacaactatcgattttcaaccagatgtttgcaaggactacaaacagacaggttactgcggatatggtgacacttgtaagtttttgcacctgagggatgatttcaaacagggatggaaattagatagggagtgggaaaatgtccaaaagaagaagcataatactctcaaaggggttaaggagatccaaatgtttaatgaagatgagctcaaagatatcccgtttaaatgcattatatgcaaaggagattacaaatcacccgtgaaaacttcttgcaatcattatttttgcgaacaatgtttcctgcaacggtcaagaagaaaaccaaattgtattatatgtggcagagacactttaggagttgctttaccagcaaagaagttgtcccaatttctggctaagatacataataatgaaagtaataaagtttagtaattgcattgcgttgactattgattgcattgatgtcgtgtgatactttcaccgaaaaaaaacacgaagcgcaataggagcggttgcatattagtccccaaagctatttaattgtgcctgaaactgttttttaagctcatcaagcataattgtatgcattgcgacgtaaccaacgtttaggcgcagtttaatcatagcccactgctaagcc

SEQ ID NO: 62 - DNA, 1906 bp, Artificial Sequence
  Sequence of the 3′-Region used for knock out of PpPNO1 and PpMNN4
cggaggaatgcaaataataatctccttaattacccactgataagctcaagagacgcggtttgaaaacgatataatgaatcatttggattttataataaaccctgacagtttttccactgtattgttttaacactcattggaagctgtattgattctaagaagctagaaatcaatacggccatacaaaagatgacattgaataagcaccggcttttttgattagcatataccttaaagcatgcattcatggctacatagttgttaaagggcttcttccattatcagtataatgaattacataatcatgcacttatatttgcccatctctgttctctcactcttgcctgggtatattctatgaaattgcgtatagcgtgtctccagttgaaccccaagcttggcgagtttgaagagaatgctaaccttgcgtattccttgcttcaggaaacattcaaggagaaacaggtcaagaagccaaacattttgatccttcccgagttagcattgactggctacaattttcaaagccagcagcggatagagccttttttggaggaaacaaccaagggagctagtacccaatgggctcaaaaagtatccaagacgtgggattgctttactttaataggatacccagaaaaaagtttagagagccctccccgtatttacaacagtgcggtacttgtatcgcctcagggaaaagtaatgaacaactacagaaagtccttcttgtatgaagctgatgaacattggggatgttcggaatcttctgatgggtttcaaacagtagatttattaattgaaggaaagactgtaaagacatcatttggaatttgcatggatttgaatccttataaatttgaagctccattcacagacttcgagttcagtggccattgcttgaaaaccggtacaagactcattttgtgcccaatggcctggttgtcccctctatcgccttccattaaaaaggatcttagtgatatagagaaaagcagacttcaaaagttctaccttgaaaaaatagataccccggaatttgacgttaattacgaattgaaaaaagatgaagtattgcccacccgtatgaatgaaacgttggaaacaattgactttgagccttcaaaaccggactactctaatataaattattggatactaaggttttttccctttctgactcatgtctataaacgagatgtgctcaaagagaatgcagttgcagtcttatgcaaccgagttggcattgagagtgatgtcttgtacggaggatcaaccacgattctaaacttcaatggtaagttagcatcgacacaagaggagctggagttgtacgggcagactaatagtctcaaccccagtgtggaagtattgggggcccttggcatgggtcaacagggaattctagtacgagacattgaattaacataatatacaatatacaataaacacaaataaagaatacaagcctgacaaaaattcacaaattattgcctagacttgtcgttatcagcagcgacctttttccaatgctcaatttcacgatatgccttttctagctctgctttaagcttctcattggaattggctaactcgttgactgcttggtcagtgatgagtttctccaaggtccatttctcgatgttgttgttttcgttttcctttaatctcttgatataatcaacagccttctttaatatctgagccttgttcgagtcccctgttggcaacagagcggccagttcctttattccgtggtttatattttctcttctacgcctttctacttctttgtgattctctttacgcatcttatgccattcttcagaaccagtggctggcttaaccgaatagccagagcctgaagaagccgcactagaagaagcagtggcattgttgactatgg

SEQ ID NO: 63 - DNA, 1224 bp, Artificial Sequence
  DNA encodes human GnTI catalytic domain (NA) Codon-optimized
tcagtcagtgctcttgatggtgacccagcaagtttgaccagagaagtgattagattggcccaagacgcagaggtggagttggagagacaacgtggactgctgcagcaaatcggagatgcattgtctagtcaaagaggtagggtgcctaccgcagctcctccagcacagcctagagtgcatgtgacccctgcaccagctgtgattcctatcttggtcatcgcctgtgacagatctactgttagaagatgtctggacaagctgttgcattacagaccatctgctgagttgttccctatcatcgttagtcaagactgtggtcacgaggagactgcccaagccatcgcctcctacggatctgctgtcactcacatcagacagcctgacctgtcatctattgctgtgccaccagaccacagaaagttccaaggttactacaagatcgctagacactacagatgggcattgggtcaagtcttcagacagtttagattccctgctgctgtggtggtggaggatgacttggaggtggctcctgacttctttgagtactttagagcaacctatccattgctgaaggcagacccatccctgtggtgtgtctctgcctggaatgacaacggtaaggagcaaatggtggacgcttctaggcctgagctgttgtacagaaccgacttctttcctggtctgggatggttgctgttggctgagttgtgggctgagttggagcctaagtggccaaaggcattctgggacgactggatgagaagacctgagcaaagacagggtagagcctgtatcagacctgagatctcaagaaccatgacctttggtagaaagggagtgtctcacggtcaattctttgaccaacacttgaagtttatcaagctgaaccagcaatttgtgcacttcacccaactggacctgtcttacttgcagagagaggcctatgacagagatttcctagctagagtctacggagctcctcaactgcaagtggagaaagtgaggaccaatgacagaaaggagttgggagaggtgagagtgcagtacactggtagggactcctttaaggctttcgctaaggctctgggtgtcatggatgaccttaagtctggagttcctagagctggttacagaggtattgtcacctttcaattcagaggtagaagagtccacttggctcctccacctacttgggagggttatgatccttcttggaattag

SEQ ID NO: 64 - DNA, 99 bp, Artificial Sequence
  DNA encodes Pp SEC12 (10) The last 9 nucleotides are the linker containing the AscI restriction site used for fusion to proteins of interest.
atgcccagaaaaatatttaactacttcattttgactgtattcatggcaattcttgctattgttttacaatggtctatagagaatggacatgggcgcgcc

SEQ ID NO: 65 - DNA, 1037 bp, Artificial Sequence
  Sequence of the PpPMA1 promoter
aaatgcgtacctcttctacgagattcaagcgaatgagaataatgtaatatgcaagatcagaaagaatgaaaggagttgaaaaaaaaaaccgttgcgttttgaccttgaatggggtggaggtttccattcaaagtaaagcctgtgtcttggtattttcggcggcacaagaaatcgtaattttcatcttctaaacgatgaagatcgcagcccaacctgtatgtagttaaccggtcggaattataagaaagattttcgatcaacaaaccctagcaaatagaaagcagggttacaactttaaaccgaagtcacaaacgataaaccactcagctcccacccaaattcattcccactagcagaaaggaattatttaatccctcaggaaacctcgatgattctcccgttcttccatgggcgggtatcgcaaaatgaggaatttttcaaatttctctattgtcaagactgtttattatctaagaaatagcccaatccgaagctcagttttgaaaaaatcacttccgcgtttcttttttacagcccgatgaatatccaaatttggaatatggattactctatcgggactgcagataatatgacaacaacgcagattacattttaggtaaggcataaacaccagccagaaatgaaacgcccactagccatggtcgaatagtccaatgaattcagatagctatggtctaaaagctgatgttttttattgggtaatggcgaagagtccagtacgacttccagcagagctgagatggccatttttgggggtattagtaactttttgagctcttttcacttcgatgaagtgtcccattcgggatataatcggatcgcgtcgttttctcgaaaatacagcttagcgtcgtccgcttgttgtaaaagcagcaccacattcctaatctcttatataaacaaaacaacccaaattatcagtgctgttttcccaccagatataagtttcttttctcttccgctttttgattttttatctctttcctttaaaaacttctttaccttaaagggcggcc

SEQ ID NO: 66 - DNA, 512 bp, Artificial Sequence
  Sequence of the PpPMA1 terminator
taagcttcacgatttgtgttccagtttatcccccctttatataccgttaaccctttccctgttgagctgactgttgttgtattaccgcaatttttccaagtttgccatgcttttcgtgttatttgaccgatgtcttttttcccaaatcaaactatatttgttaccatttaaaccaagttatcttttgtattaagagtctaagtttgttcccaggcttcatgtgagagtgataaccatccagactatgattcttgttttttattgggtttgtttgtgtgatacatctgagttgtgattcgtaaagtatgtcagtctatctagatttttaatagttaattggtaatcaatgacttgtttgttttaacttttaaattgtgggtcgtatccacgcgtttagtatagctgttcatggctgttagaggagggcgatgtttatatacagaggacaagaatgaggaggcggcgtgtatttttaaaatggagacgcgactcctgtacaccttatcggttgg

SEQ ID NO: 67 - DNA, 435 bp, Artificial Sequence
  Sequence of the PpSEC4 promoter
gaagtaaagttggcgaaactttgggaacctttggttaaaactttgtaatttttgtcgctacccattaggcagaatctgcatcttgggagggggatgtggtggcgttctgagatgtacgcgaagaatgaagagccagtggtaacaacaggcctagagagatacgggcataatgggtataacctacaagttaagaatgtagcagccctggaaaccagattgaaacgaaaaacgaaatcatttaaactgtaggatgttttggctcattgtctggaaggctggctgtttattgccctgttctttgcatgggaataagctattatatccctcacataatcccagaaaatagattgaagcaacgcgaaatccttacgtatcgaagtagccttcttacacattcacgttgtacggataagaaaactactcaaacgaacaatc

SEQ ID NO: 68 - DNA, 404 bp, Artificial Sequence
  Sequence of the PpOCH1 terminator
aatagatatagcgagattagagaatgaataccttcttctaagcgatcgtccgtcatcatagaatatcatggactgtatagttttttttttgtacatataatgattaaacggtcatccaacatctcgttgacagatctctcagtacgcgaaatccctgactatcaaagcaagaaccgatgaagaaaaaaacaacagtaacccaaacaccacaacaaacactttatcttctcccccccaacaccaatcatcaaagagatgtcggaacacaaacaccaagaagcaaaaactaaccccatataaaaacatcctggtagataatgctggtaacccgctctccttccatattctgggctacttcacgaagtctgaccggtctcagttgatcaacatgatcctcgaaatgg

SEQ ID NO: 69 - DNA, 1407 bp, Artificial Sequence
  DNA encodes Mm ManI catalytic domain (FB)
gagcccgctgacgccaccatccgtgagaagagggcaaagatcaaagagatgatgacccatgcttggaataattataaacgctatgcgtggggcttgaacgaactgaaacctatatcaaaagaaggccattcaagcagtttgtttggcaacatcaaaggagctacaatagtagatgccctggatacccttttcattatgggcatgaagactgaatttcaagaagctaaatcgtggattaaaaaatatttagattttaatgtgaatgctgaagtttctgtttttgaagtcaacatacgcttcgtcggtggactgctgtcagcctactatttgtccggagaggagatatttcgaaagaaagcagtggaacttggggtaaaattgctacctgcatttcatactccctctggaataccttgggcattgctgaatatgaaaagtgggatcgggcggaactggccctgggcctctggaggcagcagtatcctggccgaatttggaactctgcatttagagtttatgcacttgtcccacttatcaggagacccagtctttgccgaaaaggttatgaaaattcgaacagtgttgaacaaactggacaaaccagaaggcctttatcctaactatctgaaccccagtagtggacagtggggtcaacatcatgtgtcggttggaggacttggagacagcttttatgaatatttgcttaaggcgtggttaatgtctgacaagacagatctcgaagccaagaagatgtattttgatgctgttcaggccatcgagactcacttgatccgcaagtcaagtgggggactaacgtacatcgcagagtggaaggggggcctcctggaacacaagatgggccacctgacgtgctttgcaggaggcatgtttgcacttggggcagatggagctccggaagcccgggcccaacactaccttgaactcggagctgaaattgcccgcacttgtcatgaatcttataatcgtacatatgtgaagttgggaccggaagcgtttcgatttgatggcggtgtggaagctattgccacgaggcaaaatgaaaagtattacatcttacggcccgaggtcatcgagacatacatgtacatgtggcgactgactcacgaccccaagtacaggacctgggcctgggaagccgtggaggctctagaaagtcactgcagagtgaacggaggctactcaggcttacgggatgtttacattgcccgtgagagttatgacgatgtccagcaaagtttcttcctggcagagacactgaagtatttgtacttgatattttccgatgatgaccttcttccactagaacactggatcttcaacaccgaggctcatcctttccctatactccgtgaacagaagaaggaaattgatggcaaagagaaatga

SEQ ID NO: 70 - DNA, 318 bp, Artificial Sequence
  DNA encodes ScSEC12 (8) The last 9 nucleotides are the linker containing the AscI restriction site used for fusion to proteins of interest
atgaacactatccacataataaaattaccgcttaactacgccaactacacctcaatgaaacaaaaaatctctaaatttttcaccaacttcatccttattgtgctgctttcttacattttacagttctcctataagcacaatttgcattccatgcttttcaattacgcgaaggacaattttctaacgaaaagagacaccatctcttcgccctacgtagttgatgaagacttacatcaaacaactttgtttggcaaccacggtacaaaaacatctgtacctagcgtagattccataaaagtgcatggcgtggggcgcgcc

SEQ ID NO: 71 - DNA, 1250 bp, Artificial Sequence
  Sequence of the 5′-region that was used to knock into the PpADE1 locus
gagtcggccaagagatgataactgttactaagcttctccgtaattagtggtattttgtaacttttaccaataatcgtttatgaatacggatatttttcgaccttatccagtgccaaatcacgtaacttaatcatggtttaaatactccacttgaacgattcattattcagaaaaaagtcaggttggcagaaacacttgggcgctttgaagagtataagagtattaagcattaaacatctgaactttcaccgccccaatatactactctaggaaactcgaaaaattcctttccatgtgtcatcgcttccaacacactttgctgtatccttccaagtatgtccattgtgaacactgatctggacggaatcctacctttaatcgccaaaggaaaggttagagacatttatgcagtcgatgagaacaacttgctgttcgtcgcaactgaccgtatctccgcttacgatgtgattatgacaaacggtattcctgataagggaaagattttgactcagctctcagttttctggtttgattttttggcaccctacataaagaatcatttggttgcttctaatgacaaggaagtctttgctttactaccatcaaaactgtctgaagaaaaatacaaatctcaattagagggacgatccttgatagtaaaaaagcacagactgatacctttggaagccattgtcagaggttacatcactggaagtgcatggaaagagtacaagaactcaaaaactgtccatggagtcaaggttgaaaacgagaaccttcaagagagcgacgcctttccaactccgattttcacaccttcaacgaaagctgaacagggtgaacacgatgaaaacatctctattgaacaagctgctgagattgtaggtaaagacatttgtgagaaggtcgctgtcaaggcggtcgagttgtattctgctgcaaaaaacctcgcccttttgaaggggatcattattgctgatacgaaattcgaatttggactggacgaaaacaatgaattggtactagtagatgaagttttaactccagattcttctagattttggaatcaaaagacttaccaagtgggtaaatcgcaagagagttacgataagcagtttctcagagattggttgacggccaacggattgaatggcaaagagggcgtagccatggatgcagaaattgctatcaagagtaaagaaaagtatattgaagcttatgaagcaattactggcaagaaatgggcttga

SEQ ID NO: 72 - DNA, 376 bp, Artificial Sequence
  PpALG3 transcription termination sequence
atttacaattagtaatattaaggtggtaaaaacattcgtagaattgaaatgaattaatatagtatgacaatggttcatgtctataaatctccggcttcggtaccttctccccaattgaatacattgtcaaaatgaatggttgaactattaggttcgccagtttcgttattaagaaaactgttaaaatcaaattccatatcatcggttccagtgggaggaccagttccatcgccaaaatcctgtaagaatccattgtcagaacctgtaaagtcagtttgagatgaaatttttccggtctttgttgacttggaagcttcgttaaggttaggtgaaacagtttgatcaaccagcggctcccgttttcgtcgcttagtag

SEQ ID NO: 73 - DNA, 882 bp, Artificial Sequence
  Sequence of the 3′-region that was used to knock into the PpADE1 locus
atgattagtaccctcctcgcctttttcagacatctgaaatttcccttattcttccaattccatataaaatcctatttaggtaattagtaaacaatgatcataaagtgaaatcattcaagtaaccattccgtttatcgttgatttaaaatcaataacgaatgaatgtcggtctgagtagtcaatttgttgccttggagctcattggcagggggtcttttggctcagtatggaaggttgaaaggaaaacagatggaaagtggttcgtcagaaaagaggtatcctacatgaagatgaatgccaaagagatatctcaagtgatagctgagttcagaattcttagtgagttaagccatcccaacattgtgaagtaccttcatcacgaacatatttctgagaataaaactgtcaatttatacatggaatactgtgatggtggagatctctccaagctgattcgaacacatagaaggaacaaagagtacatttcagaagaaaaaatatggagtatttttacgcaggttttattagcattgtatcgttgtcattatggaactgatttcacggcttcaaaggagtttgaatcgctcaataaaggtaatagacgaacccagaatccttcgtgggtagactcgacaagagttattattcacagggatataaaacccgacaacatctttctgatgaacaattcaaaccttgtcaaactgggagattttggattagcaaaaattctggaccaagaaaacgattttgccaaaacatacgtcggtacgccgtattacatgtctcctgaagtgctgttggaccaaccctactcaccattatgtgatatatggtctcttgggtgcgtcatgtatgagctatgtgcattgaggcctcctt

SEQ ID NO: 74 - DNA, 2100 bp, Artificial Sequence
  DNA encodes ScGAL10
atgacagctcagttacaaagtgaaagtacttctaaaattgttttggttacaggtggtgctggatacattggttcacacactgtggtagagctaattgagaatggatatgactgtgttgttgctgataacctgtcgaattcaacttatgattctgtagccaggttagaggtcttgaccaagcatcacattcccttctatgaggttgatttgtgtgaccgaaaaggtctggaaaaggttttcaaagaatataaaattgattcggtaattcactttgctggtttaaaggctgtaggtgaatctacacaaatcccgctgagatactatcacaataacattttgggaactgtcgttttattagagttaatgcaacaatacaacgtttccaaatttgttttttcatcttctgctactgtctatggtgatgctacgagattcccaaatatgattcctatcccagaagaatgtcccttagggcctactaatccgtatggtcatacgaaatacgccattgagaatatcttgaatgatctttacaatagcgacaaaaaaagttggaagtttgctatcttgcgttattttaacccaattggcgcacatccctctggattaatcggagaagatccgctaggtataccaaacaatttgttgccatatatggctcaagtagctgttggtaggcgcgagaagctttacatcttcggagacgattatgattccagagatggtaccccgatcagggattatatccacgtagttgatctagcaaaaggtcatattgcagccctgcaatacctagaggcctacaatgaaaatgaaggtttgtgtcgtgagtggaacttgggttccggtaaaggttctacagtttttgaagtttatcatgcattctgcaaagcttctggtattgatcttccatacaaagttacgggcagaagagcaggtgatgttttgaacttgacggctaaaccagatagggccaaacgcgaactgaaatggcagaccgagttgcaggttgaagactcctgcaaggatttatggaaatggactactgagaatccttttggttaccagttaaggggtgtcgaggccagattttccgctgaagatatgcgttatgacgcaagatttgtgactattggtgccggcaccagatttcaagccacgtttgccaatttgggcgccagcattgttgacctgaaagtgaacggacaatcagttgttcttggctatgaaaatgaggaagggtatttgaatcctgatagtgcttatataggcgccacgatcggcaggtatgctaatcgtatttcgaagggtaagtttagtttatgcaacaaagactatcagttaaccgttaataacggcgttaatgcgaatcatagtagtatcggttctttccacagaaaaagatttttgggacccatcattcaaaatccttcaaaggatgtttttaccgccgagtacatgctgatagataatgagaaggacaccgaatttccaggtgatctattggtaaccatacagtatactgtgaacgttgcccaaaaaagtttggaaatggtatataaaggtaaattgactgctggtgaagcgacgccaataaatttaacaaatcatagttatttcaatctgaacaagccatatggagacactattgagggtacggagattatggtgcgttcaaaaaaatctgttgatgtcgacaaaaacatgattcctacgggtaatatcgtcgatagagaaattgctacctttaactctacaaagccaacggtcttaggccccaaaaatccccagtttgattgttgttttgtggtggatgaaaatgctaagccaagtcaaatcaatactctaaacaatgaattgacgcttattgtcaaggcttttcatcccgattccaatattacattagaagttttaagtacagagccaacttatcaattttataccggtgatttcttgtctgctggttacgaagcaagacaaggttttgcaattgagcctggtagatacattgatgctatcaatcaagagaactggaaagattgtgtaaccttgaaaaacggtgaaacttacgggtccaagattgtctacagattttcctga

SEQ ID NO: 75 - DNA, 1068 bp, Artificial Sequence
  DNA encodes human GalT codon optimized (XB)
ggtagagatttgtctagattgccacagttggttggtgtttccactccattgcaaggaggttctaactctgctgctgctattggtcaatcttccggtgagttgagaactggtggagctagaccacctccaccattgggagcttcctctcaaccaagaccaggtggtgattcttctccagttgttgactctggtccaggtccagcttctaacttgacttccgttccagttccacacactactgctttgtccttgccagcttgtccagaagaatccccattgttggttggtccaatgttgatcgagttcaacatgccagttgacttggagttggttgctaagcagaacccaaacgttaagatgggtggtagatacgctccaagagactgtgtttccccacacaaagttgctatcatcatcccattcagaaacagacaggagcacttgaagtactggttgtactacttgcacccagttttgcaaagacagcagttggactacggtatctacgttatcaaccaggctggtgacactattttcaacagagctaagttgttgaatgttggtttccaggaggctttgaaggattacgactacacttgtttcgttttctccgacgttgacttgattccaatgaacgaccacaacgcttacagatgtttctcccagccaagacacatttctgttgctatggacaagttcggtttctccttgccatacgttcaatacttcggtggtgtttccgctttgtccaagcagcagttcttgactatcaacggtttcccaaacaattactggggatggggtggtgaagatgacgacatctttaacagattggttttcagaggaatgtccatctctagaccaaacgctgttgttggtagatgtagaatgatcagacactccagagacaagaagaacgagccaaacccacaaagattcgacagaatcgctcacactaaggaaactatgttgtccgacggattgaactccttgacttaccaggttttggacgttcagagatacccattgtacactcagatcactgttgacatcggtactccatcctag

SEQ ID NO: 76 - DNA, 183 bp, Artificial Sequence
  DNA encodes ScMnt1 (Kre2) (33)
atggccctctttctcagtaagagactgttgagatttaccgtcattgcaggtgcggttattgttctcctcctaacattgaattccaacagtagaactcagcaatatattccgagttccatctccgctgcatttgattttacctcaggatctatatcccctgaacaacaagtcatcgggcgcgcc

SEQ ID NO: 77 - DNA, 1074 bp, Artificial Sequence
  DNA encodes DmUGT
atgaatagcatacacatgaacgccaatacgctgaagtacatcagcctgctgacgctgaccctgcagaatgccatcctgggcctcagcatgcgctacgcccgcacccggccaggcgacatcttcctcagctccacggccgtactcatggcagagttcgccaaactgatcacgtgcctgttcctggtcttcaacgaggagggcaaggatgcccagaagtttgtacgctcgctgcacaagaccatcattgcgaatcccatggacacgctgaaggtgtgcgtcccctcgctggtctatatcgttcaaaacaatctgctgtacgtctctgcctcccatttggatgcggccacctaccaggtgacgtaccagctgaagattctcaccacggccatgttcgcggttgtcattctgcgccgcaagctgctgaacacgcagtggggtgcgctgctgctcctggtgatgggcatcgtcctggtgcagttggcccaaacggagggtccgacgagtggctcagccggtggtgccgcagctgcagccacggccgcctcctctggcggtgctcccgagcagaacaggatgctcggactgtgggccgcactgggcgcctgcttcctctccggattcgcgggcatctactttgagaagatcctcaagggtgccgagatctccgtgtggatgcggaatgtgcagttgagtctgctcagcattcccttcggcctgctcacctgtttcgttaacgacggcagtaggatcttcgaccagggattcttcaagggctacgatctgtttgtctggtacctggtcctgctgcaggccggcggtggattgatcgttgccgtggtggtcaagtacgcggataacattctcaagggcttcgccacctcgctggccatcatcatctcgtgcgtggcctccatatacatcttcgacttcaatctcacgctgcagttcagcttcggagctggcctggtcatcgcctccatatttctctacggctacgatccggccaggtcggcgccgaagccaactatgcatggtcctggcggcgatgaggagaagctgctgccgcgcgtctag

SEQ ID NO: 78 - DNA, 798 bp, Artificial Sequence
  Sequence of the PpOCH1 promoter
tggacacaggagactcagaaacagacacagagcgttctgagtcctggtgctcctgacgtaggcctagaacaggaattattggctttatttgtttgtccatttcataggcttggggtaatagatagatgacagagaaatagagaagacctaatattttttgttcatggcaaatcgcgggttcgcggtcgggtcacacacggagaagtaatgagaagagctggtaatctggggtaaaagggttcaaaagaaggtcgcctggtagggatgcaatacaaggttgtcttggagtttacattgaccagatgatttggctttttctctgttcaattcacatttttcagcgagaatcggattgacggagaaatggcggggtgtggggtggatagatggcagaaatgctcgcaatcaccgcgaaagaaagactttatggaatagaactactgggtggtgtaaggattacatagctagtccaatggagtccgttggaaaggtaagaagaagctaaaaccggctaagtaactagggaagaatgatcagactttgatttgatgaggtctgaaaatactctgctgctttttcagttgctttttccctgcaacctatcattttccttttcataagcctgccttttctgttttcacttatatgagttccgccgagacttccccaaattctctcctggaacattctctatcgctctccttccaagttgcgccccctggcactgcctagtaatattaccacgcgacttatattcagttccacaatttccagtgttcgtagcaaatatcatcagcc

SEQ ID NO: 79 - DNA, 302 bp, Artificial Sequence
  PpALG12 transcription termination sequence
aatatatacctcatttgttcaatttggtgtaaagagtgtggcggatagacttcttgtaaatcaggaaagctacaattccaattgctgcaaaaaataccaatgcccataaaccagtatgagcggtgccttcgacggattgcttactttccgaccctttgtcgtttgattcttctgcctttggtgagtcagtttgtttcgactttatatctgactcatcaacttcctttacggttgcgtttttaatcataattttagccgttggcttattatcccttgagttggtaggagttttgatgatgctg

SEQ ID NO: 80 - DNA, 461 bp, Artificial Sequence
  Sequence of the 5′-Region used for knock out of PpHIS1
taactggccctttgacgtttctgacaatagttctagaggagtcgtccaaaaactcaactctgacttgggtgacaccaccacgggatccggttcttccgaggaccttgatgaccttggctaatgtaactggagttttagtatccattttaagatgtgtgtttctgtaggttctgggttggaaaaaaattttagacaccagaagagaggagtgaactggtttgcgtgggtttagactgtgtaaggcactactctgtcgaagttttagataggggttacccgctccgatgcatgggaagcgattagcccggctgttgcccgtttggtttttgaagggtaattttcaatatctctgtttgagtcatcaatttcatattcaaagattcaaaaacaaaatctggtccaaggagcgcatttaggattatggagttggcgaatcacttgaacgatagactattatttgc

SEQ ID NO: 81 - DNA, 1841 bp, Artificial Sequence
  Sequence of the 3′-Region used for knock out of PpHIS1
gtgacattcttgtctttgagatcagtaattgtagagcatagatagaataatattcaagaccaacggcttctcttcggaagctccaagtagcttatagtgatgagtaccggcatatatttataggcttaaaatttcgagggttcactatattcgtttagtgggaagagttcctttcactcttgttatctatattgtcagcgtggactgtttataactgtaccaacttagtttctttcaactccaggttaagagacataaatgtcctttgatgctgacaataatcagtggaattcaaggaaggacaatcccgacctcaatctgttcattaatgaagagttcgaatcgtccttaaatcaagcgctagactcaattgtcaatgagaaccctttctttgaccaagaaactataaatagatcgaatgacaaagttggaaatgagtccattagcttacatgatattgagcaggcagaccaaaataaaccgtcctttgagagcgatattgatggttcggcgccgttgataagagacgacaaattgccaaagaaacaaagctgggggctgagcaattttttttcaagaagaaatagcatatgtttaccactacatgaaaatgattcaagtgttgttaagaccgaaagatctattgcagtgggaacaccccatcttcaatactgcttcaatggaatctccaatgccaagtacaatgcatttacctttttcccagtcatcctatacgagcaattcaaattttttttcaatttatactttactttagtggctctctctcaagcgataccgcaacttcgcattggatatctttcttcgtatgtcgtcccacttttgtttgtactcatagtgaccatgtcaaaagaggcgatggatgatattcaacgccgaagaagggatagagaacagaacaatgaaccatatgaggttctgtccagcccatcaccagttttgtccaaaaacttaaaatgtggtcacttggttcgattgcataagggaatgagagtgcccgcagatatggttcttgtccagtcaagcgaatccaccggagagtcatttatcaagacagatcagctggatggtgagactgattggaagcttcggattgtttctccagttacacaatcgttaccaatgactgaacttcaaaatgtcgccatcactgcaagcgcaccctcaaaatcaattcactcctttcttggaagattgacctacaatgggcaatcatatggtcttacgatagacaacacaatgtggtgtaatactgtattagcttctggttcagcaattggttgtataatttacacaggtaaagatactcgacaatcgatgaacacaactcagcccaaactgaaaacgggcttgttagaactggaaatcaatagtttgtccaagatcttatgtgtttgtgtgtttgcattatctgtcatcttagtgctattccaaggaatagctgatgattggtacgtcgatatcatgcggtttctcattctattctccactattatcccagtgtctctgagagttaaccttgatcttggaaagtcagtccatgctcatcaaatagaaactgatagctcaatacctgaaaccgttgttagaactagtacaataccggaagacctgggaagaattgaatacctattaagtgacaaaactggaactcttactcaaaatgatatggaaatgaaaaaactacacctaggaacagtctcttatgctggtgataccatggatattatttctgatcatgttaaaggtcttaataacgctaaaacatcgaggaaagatcttggtatgagaataagagatttggttacaactctggccatctg

SEQ ID NO: 82 - DNA, 3105 bp, Artificial Sequence
  DNA encodes Drosophila melanogaster ManII codon-optimized (KD)
agagacgatccaattagacctccattgaaggttgctagatccccaagaccaggtcaatgtcaagatgttgttcaggacgtcccaaacgttgatgtccagatgttggagttgtacgatagaatgtccttcaaggacattgatggtggtgtttggaagcagggttggaacattaagtacgatccattgaagtacaacgctcatcacaagttgaaggtcttcgttgtcccacactcccacaacgatcctggttggattcagaccttcgaggaatactaccagcacgacaccaagcacatcttgtccaacgctttgagacatttgcacgacaacccagagatgaagttcatctgggctgaaatctcctacttcgctagattctaccacgatttgggtgagaacaagaagttgcagatgaagtccatcgtcaagaacggtcagttggaattcgtcactggtggatgggtcatgccagacgaggctaactcccactggagaaacgttttgttgcagttgaccgaaggtcaaacttggttgaagcaattcatgaacgtcactccaactgcttcctgggctatcgatccattcggacactctccaactatgccatacattttgcagaagtctggtttcaagaatatgttgatccagagaacccactactccgttaagaaggagttggctcaacagagacagttggagttcttgtggagacagatctgggacaacaaaggtgacactgctttgttcacccacatgatgccattctactcttacgacattcctcatacctgtggtccagatccaaaggtttgttgtcagttcgatttcaaaagaatgggttccttcggtttgtcttgtccatggaaggttccacctagaactatctctgatcaaaatgttgctgctagatccgatttgttggttgatcagtggaagaagaaggctgagttgtacagaaccaacgtcttgttgattccattgggtgacgacttcagattcaagcagaacaccgagtgggatgttcagagagtcaactacgaaagattgttcgaacacatcaactctcaggctcacttcaatgtccaggctcagttcggtactttgcaggaatacttcgatgctgttcaccaggctgaaagagctggacaagctgagttcccaaccttgtctggtgacttcttcacttacgctgatagatctgataactactggtctggttactacacttccagaccataccataagagaatggacagagtcttgatgcactacgttagagctgctgaaatgttgtccgcttggcactcctgggacggtatggctagaatcgaggaaagattggagcaggctagaagagagttgtccttgttccagcaccacgacggtattactggtactgctaaaactcacgttgtcgtcgactacgagcaaagaatgcaggaagctttgaaagcttgtcaaatggtcatgcaacagtctgtctacagattgttgactaagccatccatctactctccagacttctccttctcctacttcactttggacgactccagatggccaggttctggtgttgaggactctagaactaccatcatcttgggtgaggatatcttgccatccaagcatgttgtcatgcacaacaccttgccacactggagagagcagttggttgacttctacgtctcctctccattcgtttctgttaccgacttggctaacaatccagttgaggctcaggtttctccagtttggtcttggcaccacgacactttgactaagactatccacccacaaggttccaccaccaagtacagaatcatcttcaaggctagagttccaccaatgggtttggctacctacgttttgaccatctccgattccaagccagagcacacctcctacgcttccaatttgttgcttagaaagaacccaacttccttgccattgggtcaatacccagaggatgtcaagttcggtgatccaagagagatctccttgagagttggtaacggtccaaccttggctttctctgagcagggtttgttgaagtccattcagttgactcaggattctccacatgttccagttcacttcaagttcttgaagtacggtgttagatctcatggtgatagatctggtgcttacttgttcttgccaaatggtccagcttctccagtcgagttgggtcagccagttgtcttggtcactaagggtaaattggagtcttccgtttctgttggtttgccatctgtcgttcaccagaccatcatgagaggtggtgctccagagattagaaatttggtcgatattggttctttggacaacactgagatcgtcatgagattggagactcatatcgactctggtgatatcttctacactgatttgaatggattgcaattcatcaagaggagaagattggacaagttgccattgcaggctaactactacccaattccatctggtatgttcattgaggatgctaataccagattgactttgttgaccggtcaaccattgggtggatcttctttggcttctggtgagttggagattatgcaagatagaagattggcttctgatgatgaaagaggtttgggtcagggtgttttggacaacaagccagttttgcatatttacagattggtcttggagaaggttaacaactgtgtcagaccatctaagttgcatccagctggttacttgacttctgctgctcacaaagcttctcagtctttgttggatccattggacaagttcatcttcgctgaaaatgagtggatcggtgctcagggtcaattcggtggtgatcatccatctgctagagaggatttggatgtctctgtcatgagaagattgaccaagtcttctgctaaaacccagagagttggttacgttttgcacagaaccaatttgatgcaatgtggtactccagaggagcatactcagaagttggatgtctgtcacttgttgccaaatgttgctagatgtgagagaactaccttgactttcttgcagaatttggagcacttggatggtatggttgctccagaagtttgtccaatggaaaccgctgcttacgtctcttctcactcttcttga

SEQ ID NO: 83 - DNA, 108 bp, Artificial Sequence
  DNA encodes Mnn2 leader (53)
atgctgcttaccaaaaggttttcaaagctgttcaagctgacgttcatagttttgatattgtgcgggctgttcgtcattacaaacaaatacatggatgagaacacgtcg

SEQ ID NO: 84 - DNA, 1729 bp, Artificial Sequence
  DNA encodes PpHIS1 auxotrophic marker
caagttgcgtccggtatacgtaacgtctcacgatgatcaaagataatacttaatcttcatggtctactgaataactcatttaaacaattgactaattgtacattatattgaacttatgcatcctattaacgtaatcttctggcttctctctcagactccatcagacacagaatatcgttctctctaactggtcctttgacgtttctgacaatagttctagaggagtcgtccaaaaactcaactctgacttgggtgacaccaccacgggatccggttcttccgaggaccttgatgaccttggctaatgtaactggagttttagtatccattttaagatgtgtgtttctgtaggttctgggttggaaaaaaattttagacaccagaagagaggagtgaactggtttgcgtgggtttagactgtgtaaggcactactctgtcgaagttttagataggggttacccgctccgatgcatgggaagcgattagcccggctgttgcccgtttggtttttgaagggtaattttcaatatctctgtttgagtcatcaatttcatattcaaagattcaaaaacaaaatctggtccaaggagcgcatttaggattatggagttggcgaatcacttgaacgatagactattatttgctgttcctaaagagggcagattgtatgagaaatgcgttgaattacttaggggatcagatattcagtttcgaagatccagtagattggatatagctttgtgcactaacctgcccctggcattggttttccttccagctgctgacattcccacgtttgtaggagagggtaaatgtgatttgggtataactggtattgaccaggttcaggaaagtgacgtagatgtcatacctttattagacttgaatttcggtaagtgcaagttgcagattcaagttcccgagaatggtgacttgaaagaacctaaacagctaattggtaaagaaattgtttcctcctttactagcttaaccaccaggtactttgaacaactggaaggagttaagcctggtgagccactaaagacaaaaatcaaatatgttggagggtctgttgaggcctcttgtgccctaggagttgccgatgctattgtggatcttgttgagagtggagaaaccatgaaagcggcagggctgatcgatattgaaactgttctttctacttccgcttacctgatctcttcgaagcatcctcaacacccagaactgatggatactatcaaggagagaattgaaggtgtactgactgctcagaagtatgtcttgtgtaattacaacgcacctagaggtaaccttcctcagctgctaaaactgactccaggcaagagagctgctaccgtttctccattagatgaagaagattgggtgggagtgtcctcgatggtagagaagaaagatgttggaagaatcatggacgaattaaagaaacaaggtgccagtgacattcttgtctttgagatcagtaattgtagagcatagatagaataatattcaagaccaacggcttctcttcggaagctccaagtagcttatagtgatgagtaccggcatatatttataggcttaaaatttcgagggttcactatattcgtttagtgggaagagttcctttcactcttgttatctatattgtcagcgtggactgtttataactgtaccaacttagtttctttcaactccaggttaagagacataaatgtcctttgatgc

SEQ ID NO: 85 - DNA, 1068 bp, Artificial Sequence
  DNA encodes Rat GnT II  (TC) Codon-optimized
tccttggtttaccaattgaacttcgaccagatgttgagaaacgttgacaaggacggtacttggtctcctggtgagttggttttggttgttcaggttcacaacagaccagagtacttgagattgttgatcgactccttgagaaaggctcaaggtatcagagaggttttggttatcttctcccacgatttctggtctgctgagatcaactccttgatctcctccgttgacttctgtccagttttgcaggttttcttcccattctccatccaattgtacccatctgagttcccaggttctgatccaagagactgtccaagagacttgaagaagaacgctgctttgaagttgggttgtatcaacgctgaatacccagattctttcggtcactacagagaggctaagttctcccaaactaagcatcattggtggtggaagttgcactttgtttgggagagagttaaggttttgcaggactacactggattgatcttgttcttggaggaggatcattacttggctccagacttctaccacgttttcaagaagatgtggaagttgaagcaacaagagtgtccaggttgtgacgttttgtccttgggaacttacactactatcagatccttctacggtatcgctgacaaggttgacgttaagacttggaagtccactgaacacaacatgggattggctttgactagagatgcttaccagaagttgatcgagtgtactgacactttctgtacttacgacgactacaactgggactggactttgcagtacttgactttggcttgtttgccaaaagtttggaaggttttggttccacaggctccaagaattttccacgctggtgactgtggaatgcaccacaagaaaacttgtagaccatccactcagtccgctcaaattgagtccttgttgaacaacaacaagcagtacttgttcccagagactttggttatcggagagaagtttccaatggctgctatttccccaccaagaaagaatggtggatggggtgatattagagaccacgagttgtgtaaatcctacagaagattgcagtag

SEQ ID NO: 86 - DNA, 300 bp, Artificial Sequence
  DNA encodes Mnn2 leader (54) The last 9 nucleotides are the linker containing the AscI restriction site)
atgctgcttaccaaaaggttttcaaagctgttcaagctgacgttcatagttttgatattgtgcgggctgttcgtcattacaaacaaatacatggatgagaacacgtcggtcaaggagtacaaggagtacttagacagatatgtccagagttactccaataagtattcatcttcctcagacgccgccagcgctgacgattcaaccccattgagggacaatgatgaggcaggcaatgaaaagttgaaaagcttctacaacaacgttttcaactttctaatggttgattcgcccgggcgcgcc

SEQ ID NO: 87 - DNA, 1373 bp, Artificial Sequence
  Sequence of the 5′-Region used for knock out of PpARG1
gatctggccttccctgaatttttacgtccagctatacgatccgttgtgactgtatttcctgaaatgaagtttcaacctaaagttttggttgtacttgctccacctaccacggaaactaatatcgaaaccaatgaaaaagtagaactggaatcgtcaatcgaaattcgcaaccaagtggaacccaaagacttgaatctttctaaagtctattctagtgacactaatggcaacagaagatttgagctgacttttcaaatgaatctcaataatgcaatatcaacatcagacaatcaatgggctttgtctagtgacacaggatcaattatagtagtgtcttctgcaggaagaataacttccccgatcctagaagtcggggcatccgtctgtgtcttaagatcgtacaacgaacaccttttggcaataacttgtgaaggaacatgcttttcatggaatttaaagaagcaagaatgtgttctaaacagcatttcattagcacctatagtcaattcacacatgctagttaagaaagttggagatgcaaggaactattctattgtatctgccgaaggagacaacaatccgttaccccagattctagactgcgaactttccaaaaatggcgctccaattgtggctcttagcacgaaagacatctactcttattcaaagaaaatgaaatgctggatccatttgattgattcgaaatactttgaattgttgggtgctgacaatgcactgtttgagtgtgtggaagcgctagaaggtccaattggaatgctaattcatagattggtagatgagttcttccatgaaaacactgccggtaaaaaactcaaactttacaacaagcgagtactggaggacctttcaaattcacttgaagaactaggtgaaaatgcgtctcaattaagagagaaacttgacaaactctatggtgatgaggttgaggcttcttgacctcttctctctatctgcgtttctttttttttttttttttttttttttttcagttgagccagaccgcgctaaacgcataccaattgccaaatcaggcaattgtgagacagtggtaaaaaagatgcctgcaaagttagattcacacagtaagagagatcctactcataaatgaggcgcttatttagtagctagtgatagccactgcggttctgctttatgctatttgttgtatgccttactatctttgtttggctcctttttcttgacgttttccgttggagggactccctattctgagtcatgagccgcacagattatcgcccaaaattgacaaaatcttctggcgaaaaaagtataaaaggagaaaaaagctcacccttttccagcgtagaaagtatatatcagtcattgaagac

SEQ ID NO: 88 - DNA, 1470 bp, Artificial Sequence
  Sequence of the 3′-Region used for knock out of PpARG1
gggactttaactcaagtaaaaggatagttgtacaattatatatacgaagaataaatcattacaaaaagtattcgtttctttgattcttaacaggattcattttctgggtgtcatcaggtacagcgctgaatatcttgaagttaacatcgagctcatcatcgacgttcatcacactagccacgtttccgcaacggtagcaataattaggagcggaccacacagtgacgacatctttctctttgaaatggtatctgaagccttccatgaccaattgatgggctctagcgatgagttgcaagttattaatgtggttgaactcacgtgctactcgagcaccgaataaccagccagctccacgaggagaaacagcccaactgtcgacttcatctgggtcagaccaaaccaagtcacaaaatcctccttcatgagggacctcttgcgctcggctgagaactctgatttgatctaacatgcgaatatcgggagagagaccaccatggatacataatattttaccatcaatgatggcactaagggttaaaaagtcgaacacctggcaacagtacttccagacagtggtggaaccatatttattgagacattcctcataaaatccataaacctgagtgatctgtctggattcatgatttccccttaccaatgtgatatgttgaggaaacttaatttttaaaatcatgagtaacgtgaacgtctccaacgagaaatagcctctatccacatagtctcctaggaagatatagttctgttttattccattagaggaggatccgggaaacccaccactaatcttgaaaagttccagtagatcgtgaaattggccgtgaatatctccgcatactgtcactggactctgcactggctgtatattggattcctccatcagcaaatccttcacccgttcgcaaagatgcttcatatcattttcacttaaagccttgcagcttttgacttcttcaaaccactgatctggtcctctttctggcatgattaaggtctataatatttctgagctgagatgtaaaaaaaaataataaaaatggggagtgaaaaagtgtgtagcttttaggagtttgggattgataccccaaaatgatctttatgagaattaaaaggtagatacgcttttaataagaacacctatctatagtactttgtggtcttgagtaattgagatgttcagcttctgaggtttgccgttattctgggatagtagtgcgcgaccaaacaacccgccaggcaaagtgtgttgtgctcgaagacgattgccagaagagtaagtccgtcctgcctcagatgttacacactttcttccctagacagtcgatgcatcatcggatttaaacctgaaactttgatgccatgatacgcctagtcacgtcgactgagattttagataagccccgatccctttagtacattcctgttatccatggatggaatggcctgata

SEQ ID NO: 89 - DNA, 1043 bp, Artificial Sequence
  Sequence of the 5′-Region used for knock out of BMT4
aagcttgttcaccgttgggacttttccgtggacaatgttgactactccaggagggattccagctttctctactagctcagcaataatcaatgcagccccaggcgcccgttctgatggcttgatgaccgttgtattgcctgtcactatagccaggggtagggtccataaaggaatcatagcagggaaattaaaagggcatattgatgcaatcactcccaatggctctcttgccattgaagtctccatatcagcactaacttccaagaaggaccccttcaagtctgacgtgatagagcacgcttgctctgccacctgtagtcctctcaaaacgtcaccttgtgcatcagcaaagactttaccttgctccaatactatgacggaggcaattctgtcaaaattctctctcagcaattcaaccaacttgaaagcaaattgctgtctcttgatgatggagacttttttccaagattgaaatgcaatgtgggacgactcaattgcttcttccagctcctcttcggttgattgaggaacttttgaaaccacaaaattggtcgttgggtcatgtacatcaaaccattctgtagatttagattcgacgaaagcgttgttgatgaaggaaaaggttggatacggtttgtcggtctctttggtatggccggtggggtatgcaattgcagtagaagataattggacagccattgttgaaggtagagaaaaggtcagggaacttgggggttatttataccattttaccccacaaataacaactgaaaagtacccattccatagtgagaggtaaccgacggaaaaagacgggcccatgttctgggaccaatagaactgtgtaatccattgggactaatcaacagacgattggcaatataatgaaatagttcgttgaaaagccacgtcagctgtcttttcattaactttggtcggacacaacattttctactgttgtatctgtcctactttgcttatcatctgccacagggcaagtggatttccttctcgcgcggctgggtgaaaacggttaacgtgaa

SEQ ID NO: 90 - DNA, 695 bp, Artificial Sequence
  Sequence of the 3′-Region used for knock out of BMT4
gccttgggggacttcaagtctttgctagaaactagatgaggtcaggccctcttatggttgtgtcccaattgggcaatttcactcacctaaaaagcatgacaattatttagcgaaataggtagtatattttccctcatctcccaagcagtttcgtttttgcatccatatctctcaaatgagcagctacgactcattagaaccagagtcaagtaggggtgagctcagtcatcagccttcgtttctaaaacgattgagttcttttgttgctacaggaagcgccctagggaactttcgcactttggaaatagattttgatgaccaagagcgggagttgatattagagaggctgtccaaagtacatgggatcaggccggccaaattgattggtgtgactaaaccattgtgtacttggacactctattacaaaagcgaagatgatttgaagtattacaagtcccgaagtgttagaggattctatcgagcccagaatgaaatcatcaaccgttatcagcagattgataaactcttggaaagcggtatcccattttcattattgaagaactacgataatgaagatgtgagagacggcgaccctctgaacgtagacgaagaaacaaatctacttttggggtacaatagagaaagtgaatcaagggaggtatttgtggccataatactcaactctatcattaatg

SEQ ID NO: 91 - DNA, 411 bp, Artificial Sequence
  Sequence of the 5′-Region used for knock out of BMT1
catatggtgagagccgttctgcacaactagatgttttcgagcttcgcattgtttcctgcagctcgactattgaattaagatttccggatatctccaatctcacaaaaacttatgttgaccacgtgctttcctgaggcgaggtgttttatatgcaagctgccaaaaatggaaaacgaatggccatttttcgcccaggcaaattattcgattactgctgtcataaagacagtgttgcaaggctcacatttttttttaggatccgagataaagtgaatacaggacagcttatctctatatcttgtaccattcgtgaatcttaagagttcggttagggggactctagttgagggttggcactcacgtatggctgggcgcagaaataaaattcaggcgcagcagcacttatcgatg

SEQ ID NO: 92 - DNA, 692 bp, Artificial Sequence
  Sequence of the 5′-Region used for knock out of BMT1
gaattcacagttataaataaaaacaaaaactcaaaaagtttgggctccacaaaataacttaatttaaatttttgtctaataaatgaatgtaattccaagattatgtgatgcaagcacagtatgcttcagccctatgcagctactaatgtcaatctcgcctgcgagcgggcctagattttcactacaaatttcaaaactacgcggatttattgtctcagagagcaatttggcatttctgagcgtagcaggaggcttcataagattgtataggaccgtaccaacaaattgccgaggcacaacacggtatgctgtgcacttatgtggctacttccctacaacggaatgaaaccttcctctttccgcttaaacgagaaagtgtgtcgcaattgaatgcaggtgcctgtgcgccttggtgtattgtttttgagggcccaatttatcaggcgccttttttcttggttgttttcccttagcctcaagcaaggttggtctatttcatctccgcttctataccgtgcctgatactgttggatgagaacacgactcaacttcctgctgctctgtattgccagtgttttgtctgtgatttggatcggagtcctccttacttggaatgataataatcttggcggaatctccctaaacggaggcaaggattctgcctatgatgatctgctatcattgggaagctt

SEQ ID NO: 93 - DNA, 546 bp, Artificial Sequence
  Sequence of the 5′-Region used for knock out of BMT3
gatatctccctggggacaatatgtgttgcaactgttcgttgttggtgccccagtcccccaaccggtactaatcggtctatgttcccgtaactcatattcggttagaactagaacaataagtgcatcattgttcaacattgtggttcaattgtcgaacattgctggtgcttatatctacagggaagacgataagcctttgtacaagagaggtaacagacagttaattggtatttctttgggagtcgttgccctctacgttgtctccaagacatactacattctgagaaacagatggaagactcaaaaatgggagaagcttagtgaagaagagaaagttgcctacttggacagagctgagaaggagaacctgggttctaagaggctggactttttgttcgagagttaaactgcataattttttctaagtaaatttcatagttatgaaatttctgcagcttagtgtttactgcatcgtttactgcatcaccctgtaaataatgtgagcttttttccttccattgcttggtatcttccttgctgctgttt

SEQ ID NO: 94 - DNA, 378 bp, Artificial Sequence
  Sequence of the 3′-Region used for knock out of BMT3
acaaaacagtcatgtacagaactaacgcctttaagatgcagaccactgaaaagaattgggtcccatttttcttgaaagacgaccaggaatctgtccattttgtttactcgttcaatcctctgagagtactcaactgcagtcttgataacggtgcatgtgatgttctatttgagttaccacatgattttggcatgtcttccgagctacgtggtgccactcctatgctcaatcttcctcaggcaatcccgatggcagacgacaaagaaatttgggtttcattcccaagaacgagaatatcagattgcgggtgttctgaaacaatgtacaggccaatgttaatgctttttgttagagaaggaacaaacttttttgctgagc

SEQ ID NO: 95 - DNA, 1014 bp, Artificial Sequence
  DNA encodes Mouse CMP-sialic acid transporter (MmCST) Codon optimized
atggctccagctagagaaaacgtttccttgttcttcaagttgtactgtttggctgttatgactttggttgctgctgcttacactgttgctttgagatacactagaactactgctgaggagttgtacttctccactactgctgtttgtatcactgaggttatcaagttgttgatctccgttggtttgttggctaaggagactggttctttgggaagattcaaggcttccttgtccgaaaacgttttgggttccccaaaggagttggctaagttgtctgttccatccttggtttacgctgttcagaacaacatggctttcttggctttgtctaacttggacgctgctgtttaccaagttacttaccagttgaagatcccatgtactgctttgtgtactgttttgatgttgaacagaacattgtccaagttgcagtggatctccgttttcatgttgtgtggtggtgttactttggttcagtggaagccagctcaagcttccaaagttgttgttgctcagaacccattgttgggtttcggtgctattgctatcgctgttttgtgttccggtttcgctggtgtttacttcgagaaggttttgaagtcctccgacacttctttgtgggttagaaacatccagatgtacttgtccggtatcgttgttactttggctggtacttacttgtctgacggtgctgagattcaagagaagggattcttctacggttacacttactatgtttggttcgttatcttcttggcttccgttggtggtttgtacacttccgttgttgttaagtacactgacaacatcatgaagggattctctgctgctgctgctattgttttgtccactatcgcttccgttttgttgttcggattgcagatcacattgtcctttgctttgggagctttgttggtttgtgtttccatctacttgtacggattgccaagacaagacactacttccattcagcaagaggctacttccaaggagagaatcatcggtgtttagtag

SEQ ID NO: 96 - DNA, 2172 bp, Artificial Sequence
  DNA encodes Human UDP-GlcNAc 2-epimerase/ N-acetylmannosamine kinase (HsGNE) codon optimized
atggaaaagaacggtaacaacagaaagttgagagtttgtgttgctacttgtaacagagctgactactccaagttggctccaatcatgttcggtatcaagactgagccagagttcttcgagttggacgttgttgttttgggttcccacttgattgatgactacggtaacacttacagaatgatcgagcaggacgacttcgacatcaacactagattgcacactattgttagaggagaggacgaagctgctatggttgaatctgttggattggctttggttaagttgccagacgttttgaacagattgaagccagacatcatgattgttcacggtgacagattcgatgctttggctttggctacttccgctgctttgatgaacattagaatcttgcacatcgagggtggtgaagtttctggtactatcgacgactccatcagacacgctatcactaagttggctcactaccatgtttgttgtactagatccgctgagcaacacttgatttccatgtgtgaggaccacgacagaattttgttggctggttgtccatcttacgacaagttgttgtccgctaagaacaaggactacatgtccatcatcagaatgtggttgggtgacgacgttaagtctaaggactacatcgttgctttgcagcacccagttactactgacatcaagcactccatcaagatgttcgagttgactttggacgctttgatctccttcaacaagagaactttggttttgttcccaaacattgacgctggttccaaagagatggttagagttatgagaaagaagggtatcgaacaccacccaaacttcagagctgttaagcacgttccattcgaccaattcatccagttggttgctcatgctggttgtatgatcggtaactcctcctgtggtgttagagaagttggtgctttcggtactccagttatcaacttgggtactagacagatcggtagagagactggagaaaacgttttgcatgttagagatgctgacactcaggacaagattttgcaggctttgcacttgcaattcggaaagcagtacccatgttccaaaatctacggtgacggtaacgctgttccaagaatcttgaagtttttgaagtccatcgacttgcaagagccattgcagaagaagttctgtttcccaccagttaaggagaacatctcccaggacattgaccacatcttggagacattgtccgctttggctgttgatttgggtggaactaacttgagagttgctatcgtttccatgaagggagagatcgttaagaagtacactcagttcaacccaaagacttacgaggagagaatcaacttgatcttgcagatgtgtgttgaagctgctgctgaggctgttaagttgaactgtagaatcttgggtgttggtatctctactggtggtagagttaatccaagagagggtatcgttttgcactccactaagttgattcaggagtggaactccgttgatttgagaactccattgtccgacacattgcacttgccagtttgggttgacaacgacggtaattgtgctgctttggctgagagaaagttcggtcaaggaaagggattggagaacttcgttactttgatcactggtactggtattggtggtggtatcattcaccagcacgagttgattcacggttcttccttctgtgctgctgaattgggacacttggttgtttctttggacggtccagactgttcttgtggttcccacggttgtattgaagcttacgcatcaggaatggcattgcagagagaggctaagaagttgcacgacgaggacttgttgttggttgagggaatgtctgttccaaaggacgaggctgttggtgctttgcatttgatccaggctgctaagttgggtaatgctaaggctcagtccatcttgagaactgctggtactgctttgggattgggtgttgttaatatcttgcacactatgaacccatccttggttatcttgtccggtgttttggcttctcactacatccacatcgttaaggacgttatcagacagcaagctttgtcctccgttcaagacgttgatgttgttgtttccgacttggttgacccagctttgttgggtgctgcttccatggttttggactacactactagaagaatctactaatag

SEQ ID NO: 97 - DNA, 1854 bp, Artificial Sequence
  DNA encodes the PpARG1 auxotrophic marker
cagttgagccagaccgcgctaaacgcataccaattgccaaatcaggcaattgtgagacagtggtaaaaaagatgcctgcaaagttagattcacacagtaagagagatcctactcataaatgaggcgcttatttagtagctagtgatagccactgcggttctgctttatgctatttgttgtatgccttactatctttgtttggctcctttttcttgacgttttccgttggagggactccctattctgagtcatgagccgcacagattatcgcccaaaattgacaaaatcttctggcgaaaaaagtataaaaggagaaaaaagctcacccttttccagcgtagaaagtatatatcagtcattgaagactattatttaaataacacaatgtctaaaggaaaagtttgtttggcctactccggtggtttggatacctccatcatcctagcttggttgttggagcagggatacgaagtcgttgcctttttagccaacattggtcaagaggaagactttgaggctgctagagagaaagctctgaagatcggtgctaccaagtttatcgtcagtgacgttaggaaggaatttgttgaggaagttttgttcccagcagtccaagttaacgctatctacgagaacgtctacttactgggtacctctttggccagaccagtcattgccaaggcccaaatagaggttgctgaacaagaaggttgttttgctgttgcccacggttgtaccggaaagggtaacgatcaggttagatttgagctttccttttatgctctgaagcctgacgttgtctgtatcgccccatggagagacccagaattcttcgaaagattcgctggtagaaatgacttgctgaattacgctgctgagaaggatattccagttgctcagactaaagccaagccatggtctactgatgagaacatggctcacatctccttcgaggctggtattctagaagatccaaacactactcctccaaaggacatgtggaagctcactgttgacccagaagatgcaccagacaagccagagttctttgacgtccactttgagaagggtaagccagttaaattagttctcgagaacaaaactgaggtcaccgatccggttgagatctttttgactgctaacgccattgctagaagaaacggtgttggtagaattgacattgtcgagaacagattcatcggaatcaagtccagaggttgttatgaaactccaggtttgactctactgagaaccactcacatcgacttggaaggtcttaccgttgaccgtgaagttagatcgatcagagacacttttgttaccccaacctactctaagttgttatacaacgggttgtactttaccccagaaggtgagtacgtcagaactatgattcagccttctcaaaacaccgtcaacggtgttgttagagccaaggcctacaaaggtaatgtgtataacctaggaagatactctgaaaccgagaaattgtacgatgctaccgaatcttccatggatgagttgaccggattccaccctcaagaagctggaggatttatcacaacacaagccatcagaatcaagaagtacggagaaagtgtcagagagaagggaaagtttttgggactttaactcaagtaaaaggatagttgtacaattatatatacgaagaataaatcattacaaaaagtattcgtttctttgattcttaacaggattcattttctgggtgtcatcaggtacagcgctgaatatcttgaagttaacatcgagctcatcatcgacgttcatcacactagccacgtttccgcaacggtagcaataattaggagcggaccacacagtgacgacatc

SEQ ID NO: 98 - DNA, 1308 bp, Artificial Sequence
  DNA encodes Human CMP-sialic acid synthase (HsCSS) codon optimized
atggactctgttgaaaagggtgctgctacttctgtttccaacccaagaggtagaccatccagaggtagacctcctaagttgcagagaaactccagaggtggtcaaggtagaggtgttgaaaagccaccacacttggctgctttgatcttggctagaggaggttctaagggtatcccattgaagaacatcaagcacttggctggtgttccattgattggatgggttttgagagctgctttggactctggtgctttccaatctgtttgggtttccactgaccacgacgagattgagaacgttgctaagcaattcggtgctcaggttcacagaagatcctctgaggtttccaaggactcttctacttccttggacgctatcatcgagttcttgaactaccacaacgaggttgacatcgttggtaacatccaagctacttccccatgtttgcacccaactgacttgcaaaaagttgctgagatgatcagagaagagggttacgactccgttttctccgttgttagaaggcaccagttcagatggtccgagattcagaagggtgttagagaggttacagagccattgaacttgaacccagctaaaagaccaagaaggcaggattgggacggtgaattgtacgaaaacggttccttctacttcgctaagagacacttgatcgagatgggatacttgcaaggtggaaagatggcttactacgagatgagagctgaacactccgttgacatcgacgttgatatcgactggccaattgctgagcagagagttttgagatacggttacttcggaaaggagaagttgaaggagatcaagttgttggtttgtaacatcgacggttgtttgactaacggtcacatctacgtttctggtgaccagaaggagattatctcctacgacgttaaggacgctattggtatctccttgttgaagaagtccggtatcgaagttagattgatctccgagagagcttgttccaagcaaacattgtcctctttgaagttggactgtaagatggaggtttccgtttctgacaagttggctgttgttgacgaatggagaaaggagatgggtttgtgttggaaggaagttgcttacttgggtaacgaagtttctgacgaggagtgtttgaagagagttggtttgtctggtgctccagctgatgcttgttccactgctcaaaaggctgttggttacatctgtaagtgtaacggtggtagaggtgctattagagagttcgctgagcacatctgtttgttgatggagaaagttaataactcctgtcagaagtagtag

SEQ ID NO: 99 - DNA, 1080 bp, Artificial Sequence
  DNA encodes Human N-acetylneuraminate-9- phosphate synthase (HsSPS) codon optimized
atgccattggaattggagttgtgtcctggtagatgggttggtggtcaacacccatgtttcatcatcgctgagatcggtcaaaaccaccaaggagacttggacgttgctaagagaatgatcagaatggctaaggaatgtggtgctgactgtgctaagttccagaagtccgagttggagttcaagttcaacagaaaggctttggaaagaccatacacttccaagcactcttggggaaagacttacggagaacacaagagacacttggagttctctcacgaccaatacagagagttgcagagatacgctgaggaagttggtatcttcttcactgcttctggaatggacgaaatggctgttgagttcttgcacgagttgaacgttccattcttcaaagttggttccggtgacactaacaacttcccatacttggaaaagactgctaagaaaggtagaccaatggttatctcctctggaatgcagtctatggacactatgaagcaggtttaccagatcgttaagccattgaacccaaacttttgtttcttgcagtgtacttccgcttacccattgcaaccagaggacgttaatttgagagttatctccgagtaccagaagttgttcccagacatcccaattggttactctggtcacgagactggtattgctatttccgttgctgctgttgctttgggtgctaaggttttggagagacacatcactttggacaagacttggaagggttctgatcactctgcttctttggaacctggtgagttggctgaacttgttagatcagttagattggttgagagagctttgggttccccaactaagcaattgttgccatgtgagatggcttgtaacgagaagttgggaaagtccgttgttgctaaggttaagatcccagagggtactatcttgactatggacatgttgactgttaaagttggagagccaaagggttacccaccagaggacatctttaacttggttggtaaaaaggttttggttactgttgaggaggacgacactattatggaggagttggttgacaaccacggaaagaagatcaagtcctag

SEQ ID NO: 100 - DNA, 1092 bp, Artificial Sequence
  DNA encodes Mouse alpha-2,6-sialyl transferase catalytic domain (MmmST6) codon optimized
gtttttcaaatgccaaagtcccaggagaaagttgctgttggtccagctccacaagctgttttctccaactccaagcaagatccaaaggagggtgttcaaatcttgtcctacccaagagttactgctaaggttaagccacaaccatccttgcaagtttgggacaaggactccacttactccaagttgaacccaagattgttgaagatttggagaaactacttgaacatgaacaagtacaaggtttcctacaagggtccaggtccaggtgttaagttctccgttgaggctttgagatgtcacttgagagaccacgttaacgtttccatgatcgaggctactgacttcccattcaacactactgaatgggagggatacttgccaaaggagaacttcagaactaaggctggtccatggcataagtgtgctgttgtttcttctgctggttccttgaagaactcccagttgggtagagaaattgacaaccacgacgctgttttgagattcaacggtgctccaactgacaacttccagcaggatgttggtactaagactactatcagattggttaactcccaattggttactactgagaagagattcttgaaggactccttgtacactgagggaatcttgattttgtgggacccatctgtttaccacgctgacattccacaatggtatcagaagccagactacaacttcttcgagacttacaagtcctacagaagattgcacccatcccagccattctacatcttgaagccacaaatgccatgggaattgtgggacatcatccaggaaatttccccagacttgatccaaccaaacccaccatcttctggaatgttgggtatcatcatcatgatgactttgtgtgaccaggttgacatctacgagttcttgccatccaagagaaagactgatgtttgttactaccaccagaagttcttcgactccgcttgtactatgggagcttaccacccattgttgttcgagaagaacatggttaagcacttgaacgaaggtactgacgaggacatctacttgttcggaaaggctactttgtccggtttcagaaacaacagatgttag

SEQ ID NO: 101 - DNA, 1302 bp, Artificial Sequence
  Pp TRP2 5′ and ORF
actgggcctttagagggtgctgaagttgaccccttggtgcttctggaaaaagaactgaagggcaccagacaagcgcaacttcctggtattcctcgtctaagtggtggtgccataggatacatctcgtacgattgtattaagtactttgaaccaaaaactgaaagaaaactgaaagatgttttgcaacttccggaagcagctttgatgttgttcgacacgatcgtggcttttgacaatgtttatcaaagattccaggtaattggaaacgtttctctatccgttgatgactcggacgaagctattcttgagaaatattataagacaagagaagaagtggaaaagatcagtaaagtggtatttgacaataaaactgttccctactatgaacagaaagatattattcaaggccaaacgttcacctctaatattggtcaggaagggtatgaaaaccatgttcgcaagctgaaagaacatattctgaaaggagacatcttccaagctgttccctctcaaagggtagccaggccgacctcattgcaccctttcaacatctatcgtcatttgagaactgtcaatccttctccatacatgttctatattgactatctagacttccaagttgttggtgcttcacctgaattactagttaaatccgacaacaacaacaaaatcatcacacatcctattgctggaactcttcccagaggtaaaactatcgaagaggacgacaattatgctaagcaattgaagtcgtctttgaaagacagggccgagcacgtcatgctggtagatttggccagaaatgatattaaccgtgtgtgtgagcccaccagtaccacggttgatcgtttattgactgtggagagattttctcatgtgatgcatcttgtgtcagaagtcagtggaacattgagaccaaacaagactcgcttcgatgctttcagatccattttcccagcaggtaccgtctccggtgctccgaaggtaagagcaatgcaactcataggagaattggaaggagaaaagagaggtgtttatgcgggggccgtaggacactggtcgtacgatggaaaatcgatggacacatgtattgccttaagaacaatggtcgtcaaggacggtgtcgcttaccttcaagccggaggtggaattgtctacgattctgacccctatgacgagtacatcgaaaccatgaacaaaatgagatccaacaataacaccatcttggaggctgagaaaatctggaccgataggttggccagagacgagaatcaaagtgaatccgaagaaaacgatcaatga

SEQ ID NO: 102 - DNA, 1085 bp, Artificial Sequence
  PpTRP2 3′ region
acggaggacgtaagtaggaatttatgtaatcatgccaatacatctttagatttcttcctcttctttttaacgaaagacctccagttttgcactctcgactctctagtatcttcccatttctgttgctgcaacctcttgccttctgtttccttcaattgttcttctttcttctgttgcacttggccttcttcctccatctttcgttttttttcaagccttttcagcagttcttcttccaagagcagttctttgattttctctctccaatccaccaaaaaactggatgaattcaaccgggcatcatcaatgttccactttctttctcttatcaataatctacgtgcttcggcatacgaggaatccagttgctccctaatcgagtcatccacaaggttagcatgggcctttttcagggtgtcaaaagcatctggagctcgtttattcggagtcttgtctggatggatcagcaaagactttttgcggaaagtctttcttatatcttccggagaacaacctggtttcaaatccaagatggcatagctgtccaatttgaaagtggaaagaatcctgccaatttccttctctcgtgtcagctcgttctcctccttttgcaacaggtccacttcatctggcatttttctttatgttaactttaattattattaattataaagttgattatcgttatcaaaataatcatattcgagaaataatccgtccatgcaatatataaataagaattcataataatgtaatgataacagtacctctgatgacctttgatgaaccgcaattttctttccaatgacaagacatccctataatacaattatacagtttatatatcacaaataatcacctttttataagaaaaccgtcctctccgtaacagaacttattatccgcacgttatggttaacacactactaataccgatatagtgtatgaagtcgctacgagatagccatccaggaaacttaccaattcatcagcactttcatgatccgattgttggctttattctttgcgagacagatacttgccaatgaaataactgatcccacagatgagaatccggtgctcgt

SEQ ID NO: 103 - DNA, 1494 bp, Artificial Sequence
  DNA encodes Tr ManI catalytic domain
cgcgccggatctcccaaccctacgagggcggcagcagtcaaggccgcattccagacgtcgtggaacgcttaccaccattttgcctttccccatgacgacctccacccggtcagcaacagctttgatgatgagagaaacggctggggctcgtcggcaatcgatggcttggacacggctatcctcatgggggatgccgacattgtgaacacgatccttcagtatgtaccgcagatcaacttcaccacgactgcggttgccaaccaaggcatctccgtgttcgagaccaacattcggtacctcggtggcctgctttctgcctatgacctgttgcgaggtcctttcagctccttggcgacaaaccagaccctggtaaacagccttctgaggcaggctcaaacactggccaacggcctcaaggttgcgttcaccactcccagcggtgtcccggaccctaccgtcttcttcaaccctactgtccggagaagtggtgcatctagcaacaacgtcgctgaaattggaagcctggtgctcgagtggacacggttgagcgacctgacgggaaacccgcagtatgcccagcttgcgcagaagggcgagtcgtatctcctgaatccaaagggaagcccggaggcatggcctggcctgattggaacgtttgtcagcacgagcaacggtacctttcaggatagcagcggcagctggtccggcctcatggacagcttctacgagtacctgatcaagatgtacctgtacgacccggttgcgtttgcacactacaaggatcgctgggtccttgctgccgactcgaccattgcgcatctcgcctctcacccgtcgacgcgcaaggacttgacctttttgtcttcgtacaacggacagtctacgtcgccaaactcaggacatttggccagttttgccggtggcaacttcatcttgggaggcattctcctgaacgagcaaaagtacattgactttggaatcaagcttgccagctcgtactttgccacgtacaaccagacggcttctggaatcggccccgaaggcttcgcgtgggtggacagcgtgacgggcgccggcggctcgccgccctcgtcccagtccgggttctactcgtcggcaggattctgggtgacggcaccgtattacatcctgcggccggagacgctggagagcttgtactacgcataccgcgtcacgggcgactccaagtggcaggacctggcgtgggaagcgttcagtgccattgaggacgcatgccgcgccggcagcgcgtactcgtccatcaacgacgtgacgcaggccaacggcgggggtgcctctgacgatatggagagcttctggtttgccgaggcgctcaagtatgcgtacctgatctttgcggaggagtcggatgtgcaggtgcaggccaacggcgggaacaaatttgtctttaacacggaggcgcacccctttagcatccgttcatcatcacgacggggcggccaccttgcttaa

SEQ ID NO: 104 - DNA, 57 bp, Artificial Sequence
  DNA encodes Saccharomyces cerevisiae mating factor pre-signal peptide
atgagattcccatccatcttcactgctgttttgttcgctgcttcttctgctttggct

SEQ ID NO: 105 - protein, 19 aa, Artificial Sequence
  Saccharomyces cerevisiae mating factor pre-signal peptide
MRFPSIFTAVLFAASSALA

SEQ ID NO: 106 - DNA, 747 bp, Artificial Sequence
  Sequence of the 5′-Region used for knock out of STE13
ttgggggcctccaggacttgctgaaatttgctgactcatcttcgccatccaaggataatgagttagctaatgtgacagttaatgagtcgtcttgactaacggggaacatttcattatttatatccagagtcaatttgatagcagagtttgtggttgaaatacctatgattcgggagactttgttgtaacgaccattatccacagtttggaccgtgaaaatgtcatcgaagagagcagacgacatattatctattgtggtaagtgatagttggaagtccgactaaggcatgaaaatgagaagactgaaaatttaaagtttttgaaaacactaatcgggtaataacttggaaattacgtttacgtgcctttagctcttgtccttacccctgataatctatccatttcccgagagacaatgacatctcggacagctgagaacccgttcgatatagagcttcaagagaatctaagtccacgttcttccaattcgtccatattggaaaacattaatgagtatgctagaagacatcgcaatgattcgctttcccaagaatgtgataatgaagatgagaacgaaaatctcaattatactgataacttggccaagttttcaaagtctggagtatcaagaaagagctgtatgctaatatttggtatttgctttgttatctggctgtttctctttgccttgtatgcgagggacaatcgattttccaatttgaacgagtacgttccagattcaaacag

SEQ ID NO: 107 - DNA, 924 bp, Artificial Sequence
  Sequence of the 3′-Region used for knock out of STE13
ctactgggaaccacgagacatcactgcagtagtttccaagtggatttcagatcactcatttgtgaatcctgacaaaactgcgatatgggggtggtcttacggtgggttcactacgcttaagacattggaatatgattctggagaggttttcaaatatggtatggctgttgctccagtaactaattggcttttgtatgactccatctacactgaaagatacatgaaccttccaaaggacaatgttgaaggctacagtgaacacagcgtcattaagaaggtttccaattttaagaatgtaaaccgattcttggtttgtcacgggactactgatgataacgtgcattttcagaacacactaaccttactggaccagttcaatattaatggtgttgtgaattacgatcttcaggtgtatcccgacagtgaacatagcattgcccatcacaacgcaaataaagtgatctacgagaggttattcaagtggttagagcgggcatttaacgatagatttttgtaacattccgtacttcatgccatactatatatcctgcaaggtttccctttcagacacaataattgctttgcaattttacataccaccaattggcaaaaataatctcttcagtaagttgaatgcttttcaagccagcaccgtgagaaattgctacagcgcgcattctaacatcactttaaaattccctcgccggtgctcactggagtttccaacccttagcttatcaaaatcgggtgataactctgagttttttttttcacttctattcctaaaccttcgcccaatgctaccacctccaatcaacatcccgaaatggatagaagagaatggacatctcttgcaacctccggttaataattactgtctccacagaggaggatttacggtaatgattgtaggtgggcctaatg

SEQ ID NO: 108 - DNA, 573 bp, Artificial Sequence
  DNA encodes NatR
atgggtaccactcttgacgacacggcttaccggtaccgcaccagtgtcccgggggacgccgaggccatcgaggcactggatgggtccttcaccaccgacaccgtcttccgcgtcaccgccaccggggacggcttcaccctgcgggaggtgccggtggacccgcccctgaccaaggtgttccccgacgacgaatcggacgacgaatcggacgacggggaggacggcgacccggactcccggacgttcgtcgcgtacggggacgacggcgacctggcgggcttcgtggtcgtctcgtactccggctggaaccgccggctgaccgtcgaggacatcgaggtcgccccggagcaccgggggcacggggtcgggcgcgcgttgatggggctcgcgacggagttcgcccgcgagcggggcgccgggcacctctggctggaggtcaccaacgtcaacgcaccggcgatccacgcgtaccggcggatggggttcaccctctgcggcctggacaccgccctgtacgacggcaccgcctcggacggcgagcaggcgctctacatgagcatgccctgcccctaa

SEQ ID NO: 109 - DNA, 388 bp, Artificial Sequence
  Ashbya gossypii TEF1 promoter
gatctgtttagcttgcctcgtccccgccgggtcacccggccagcgacatggaggcccagaataccctccttgacagtcttgacgtgcgcagctcaggggcatgatgtgactgtcgcccgtacatttagcccatacatccccatgtataatcatttgcatccatacattttgatggccgcacggcgcgaagcaaaaattacggctcctcgctgcagacctgcgagcagggaaacgctcccctcacagacgcgttgaattgtccccacgccgcgcccctgtagagaaatataaaaggttaggatttgccactgaggttcttctttcatatacttccttttaaaatcttgctaggatacagttctcacatcacatccgaacataaacaacc

SEQ ID NO: 110 - DNA, 247 bp, Artificial Sequence
  Ashbya gossypii TEF1 termination sequence
taatcagtactgacaataaaaagattcttgttttcaagaacttgtcatttgtatagtttttttatattgtagttgttctattttaatcaaatgttagcgtgatttatattttttttcgcctcgacatcatctgcccagatgcgaagttaagtgcgcagaaagtaatatcatgcgtcaatcgtatgtgaatgctggtcgctatactgctgtcgattcgatactaacgccgccatccagtgtcgaaaac

SEQ ID NO: 111 - DNA, 980 bp, Artificial Sequence
  Sequence of the 5′-Region used for knock out of DAP2
cacctgggcctgttgctgctggtactgctgttggaactgttggtattgttgctgatctaaggccgcctgttccacaccgtgtgtatcgaatgcttgggcaaaatcatcgcctgccggaggccccactaccgcttgttcctcctgctcttgtttgttttgctcattgatgatatcggcgtcaatgaattgatcctcaatcgtgtggtggtggtgtcgtgattcctcttctttcttgagtgccttatccatattcctatcttagtgtaccaataattttgttaaacacacgctgttgtttatgaaaagtcgtcaaaaggttaaaaattctacttggtgtgtgtcagagaaagtagtgcagacccccagtttgttgactagttgagaaggcggctcactattgcgcgaatagcatgagaaatttgcaaacatctggcaaagtggtcaatacctgccaacctgccaatcttcgcgacggaggctgttaagcgggttgggttcccaaagtgaatggatattacgggcaggaaaaacagccccttccacactagtctttgctactgacatcttccctctcatgtatcccgaacacaagtatcgggagtatcaacggagggtgcccttatggcagtactccctgttggtgattgtactgctatacgggtctcatttgcttatcagcaccatcaacttgatacactataaccacaaaaattatcatgcacacccagtcaatagtggtatcgttcttaatgagtttgctgatgacgattcattctctttgaatggcactctgaacttggagaactggagaaatggtaccttttcccctaaatttcattccattcagtggaccgaaataggtcaggaagatgaccagggatattacattctctcttccaattcctcttacatagtaaagtctttatccgacccagactttgaatctgttctattcaacgagtctacaatcacttacaacg

SEQ ID NO: 112 - DNA, 1117 bp, Artificial Sequence
  Sequence of the 3′-Region used for knock out of DAP2
ggcagcaaagccttacgttgatgagaatagactggccatttggggttggtcttatggaggttacatgacgctaaaggttttagaacaggataaaggtgaaacattcaaatatggaatgtctgttgcccctgtgacgaattggaaattctatgattctatctacacagaaagatacatgcacactcctcaggacaatccaaactattataattcgtcaatccatgagattgataatttgaagggagtgaagaggttcttgctaatgcacggaactggtgacgacaatgttcacttccaaaatacactcaaagttctagatttatttgatttacatggtcttgaaaactatgatatccacgtgttccctgatagtgatcacagtattagatatcacaacggtaatgttatagtgtatgataagctattccattggattaggcgtgcattcaaggctggcaaataaataggtgcaaaaatattattagactttttttttcgttcgcaagttattactgtgtaccataccgatccaatccgtattgtaattcatgttctagatccaaaatttgggactctaattcatgaggtctaggaagatgatcatctctatagttttcagcggggggctcgatttgcggttggtcaaagctaacatcaaaatgtttgtcaggttcagtgaatggtaactgctgctcttgaattggtcgtctgacaaattctctaagtgatagcacttcatctacaatcatttgcttcatcgtttctatatcgtccacgacctcaaacgagaaatcgaatttggaagaacagacgggctcatcgttaggatcatgccaaaccttgagatatggatgctctaaagcctcagtaactgtaattctgtgagtgggatctaccgtgagcattcgatccagtaagtctatcgcttcagggttggcaccgggaaataactggctgaatgggatcttgggcatgaatggcagggagcgaacataatcctgggcacgctctgatctgatagactgaagtgtctcttccgaaacagtacccagcgtactcaaaatcaagttcaattgatccacatagtctcttcctctaaaaatgggtcggccaccta

SEQ ID NO: 113 - DNA, 1666 bp, Artificial Sequence
  HYGR resistance cassette
gatctgtttagcttgcctcgtccccgccgggtcacccggccagcgacatggaggcccagaataccctccttgacagtcttgacgtgcgcagctcaggggcatgatgtgactgtcgcccgtacatttagcccatacatccccatgtataatcatttgcatccatacattttgatggccgcacggcgcgaagcaaaaattacggctcctcgctgcggacctgcgagcagggaaacgctcccctcacagacgcgttgaattgtccccacgccgcgcccctgtagagaaatataaaaggttaggatttgccactgaggttcttctttcatatacttccttttaaaatcttgctaggatacagttctcacatcacatccgaacataaacaaccatgggtaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaataatcagtactgacaataaaaagattcttgttttcaagaacttgtcatttgtatagtttttttatattgtagttgttctattttaatcaaatgttagcgtgatttatattttttttcgcctcgacatcatctgcccagatgcgaagttaagtgcgcagaaagtaatatcatgcgtcaatcgtatgtgaatgctggtcgctatactgctgtcgattcgatactaacgccgccatccagtgtcgaaaacgagct

SEQ ID NO: 114 - DNA, 365 bp, Artificial Sequence
  Sequence of PpTRP5 5′ integration fragment
acgacggccaaattcatgatacacactctgtttcagctggtttggactaccctggagttggtcctgaattggctgcctggaaagcaaatggtagagcccaattttccgctgtaactgatgcccaagcattagagggattcaaaatcctgtctcaattggaagggatcattccagcactagagtctagtcatgcaatctacggcgcattgcaaattgcaaagactatgtcttcggaccagtccttagttattaatgtatctggaaggggtgataaggacgtccagagtgtagctgagattttacctaaattgggacctcaaattggatgggatttgcgtttcagcgaagacattactaaagagtga

SEQ ID NO: 115 - DNA, 613 bp, Artificial Sequence
  Sequence of PpTRP5 3′ integration fragment
tcgatagcacaatattcaacttgactgggtgttaagaactaagagctctgggaaactttgtatttattactaccaacacagtcaaattattggatgtgtttttttttccagtacatttcactgagcagtttgttatactcggtctttaatctccatatacatgcagattgtaatacagatctgaacagtttgattctgattgatcttgccaccaatattctatttttgtatcaagtaacagagtcaatgatcattggtaacgtaacggttttcgtgtatagtagttagagcccatcttgtaacctcatttcctcccatattaaagtatcagtgattcgctggaacgattaactaagaaaaaaaaaatatctgcacatactcatcagtctgtaaatctaagtcaaaactgctgtatccaatagaaatcgggatatacctggatgttttttccacataaacaaacgggagttcagcttacttatggtgttgatgcaattcagtatgatcctaccaataaaacgaaactttgggattttggctgtttgagggatcaaaagctgcacctttacaagattgacggatcgaccattagaccaaagcaaatggccaccaa

SEQ ID NO: 116 - DNA, 1213 bp, Artificial Sequence
  3′ sequence for knocking out VPS10-1
acgacgacgaggagaatatcaattttgattcccggtagatagctcacccacggtcacacacacaaacacacatacacattaacacacagagttattagttaacagagaaaactctaacaaagtatttattttcgttacgtaatccgacttttctttttaccgttttctattgctcctctcatttgcccctaaaagttgctcctcattactaaaatcaccacaccatgctcgaatatgatgttactaaatgcaaattgtagtcgtgcctcttgtggtaatactatagggaatatctctcgattactcgattctggttaattttttctttttttataggggaagtttttttttcttcccctttctctccagtttatttatttactaagaaaatccaacagataccaaccacccaaaaagatcctaaacagcctgtttttgaggagtttttcagcagctaagcttcatcagttttttaatacttaatttattgcccttcactttgtttcttgtggcttttaaggctctccggaacagcggtttcaaaatcaaatctcagttatttgtttgctccgctttgtcagttcaaagatcatggtttccgaaaacaagaatcaatcttcgattttgatggacaactccaagaagctctctccgaagcccattttgaataacaagaatgaaccgtttggcatcggcgtcgatggacttcaacatcctcaaccgactttatgccgcacagaatcggaactcttgttcaacttgagccaagtcaataaatcccaaataactttggacggtgcagttactccacctgctgatggtaatgggaatgaagcaaaaagagcaaatctcatctcttttgatgttccatcgtctcaagtgaaacatagagggtctattagtgcaaggccctcggcagtgaatgtgtcccaaattaccggggccctttctcaatccggatcttctagaaatccctacgatcaaacacagtcacctccacctagcacttacgcctccaggcagaactccacccatggaaataatatcgatagcttgcaatatttggcaacaagagatcttagtgctttaaggctggaaagagatgcttccgcacgagaagctacctcttctgcagtgtccactcctgttcagttcgatgtacccaaacaacatcatctccttcatttagaacaagacccgacaaggcccatcc

SEQ ID NO: 117 - DNA, 1632 bp, Artificial Sequence
  5′ sequence for knocking out VPS10-1
aagtgggccagattatataaatatggatcaacatgaagccttgaaagatttcaaggacaggcttaggaattacgaaaaagtttacgagactattgacgaccaggaggaagaggagaacgaacggtacaatattcagtatctgaagataatcaacgcaggaaagaagatagtcagttataacataaatgggtatttatcgtcccacaccgttttttatctcctgaatttcaatcttgcagaacgtcaaatatggttgacgacgaatggagagacagagtataaccttcaaaataggattggaggtgattccaaattaagcaatgagggatggaaatttgccaaagcattgcccaagtttatagcacagaaaagaaaagagtttcaacttagacagttgaccaaacactatatcgagactcaaacgcccattgaagacgtaccgttggaggagcacaccaagccagtcaaatattctgatctgcatttccatgtttggtcatcggctttaaagagatctactcaatcaacaacattttttccatcggaaaattactctctgaagcaattcagaacgttgaatgatctctgttgcggatcactggatggtttgactgaacaagagttcaaaagtaaatacaaagaagaataccagaattctcagactgataaactgagtttcagtttccctggtatcggtggggagtcttatttggacgtgatcaaccgtttgagaccactaatagttgaactagaaaggttgccagaacatgtcctggtcattacccaccgggtcatagtaaggattttactaggatatttcatgaatttggatagaaatctgttgacagatttggaaattttgcatgggtatgtttattgtattgagccgaaaccttatggtttagacttaaagatctggcagtatgatgaggcggacaacgagtttaatgaagttgataagctggaattcatgaaaagaagaagaaaatcgatcaacgtcaacacgacagatttcagaatgcagttaaacaaagagttgcaacaggacgctctcaataatagtcctggtaataatagtccgggcgtatcatctctatcttcatactcgtcgtcctcttccctttccgctgacgggagcgagggagaaacattaataccacaagtatcccaggcggagagctacaactttgaatttaactctctttcatcatcagtttcatcgttgaaaaggacgacatcttcttcccaacatttgagctccaatcctagttgtctgagcatgcataatgcctcattggacgagaatgacgacgaacatttaatagacccggcttctacagacgacaagctaaacatggtattacaggacaaaacgctaattaaaaagctcaaaagtttactacttgacgaggccgaaggctagacaatccacagttaattttgatactgtactttataacgagtaacatacatatcttatgtaatcatctatgtcacgtcacgtgcgcgcgacattattccgagaacttgcgccctgctagctccactgtcagagtgataacttccccaaaataggatccaactgtttccaattgcttttggaaatgtggattgaaagaaacctcatagcgt

SEQ ID NO: 118 - DNA, 934 bp, Artificial Sequence
  Pp AOX1 promoter
aacatccaaagacgaaaggttgaatgaaacctttttgccatccgacatccacaggtccattctcacacataagtgccaaacgcaacaggaggggatacactagcagcagaccgttgcaaacgcaggacctccactcctcttctcctcaacacccacttttgccatcgaaaaaccagcccagttattgggcttgattggagctcgctcattccaattccttctattaggctactaacaccatgactttattagcctgtctatcctggcccccctggcgaggttcatgtttgtttatttccgaatgcaacaagctccgcattacacccgaacatcactccagatgagggctttctgagtgtggggtcaaatagtttcatgttccccaaatggcccaaaactgacagtttaaacgctgtcttggaacctaatatgacaaaagcgtgatctcatccaagatgaactaagtttggttcgttgaaatgctaacggccagttggtcaaaaagaaacttccaaaagtcggcataccgtttgtcttgtttggtattgattgacgaatgctcaaaaataatctcattaatgcttagcgcagtctctctatcgcttctgaaccccggtgcacctgtgccgaaacgcaaatggggaaacacccgctttttggatgattatgcattgtctccacattgtatgcttccaagattctggtgggaatactgctgatagcctaacgttcatgatcaaaatttaactgttctaacccctacttgacagcaatatataaacagaaggaagctgccctgtcttaaacctttttttttatcatcattattagcttactttcataattgcgactggttccaattgacaagcttttgattttaacgacttttaacgacaacttgagaagatcaaaaaacaactaattattcgaaacg

SEQ ID NO: 119 - DNA, 1231 bp, Artificial Sequence
  Sequence of the 5′-region that was used to knock into the PpPRO1 locus
gaagggccatcgaattgtcatcgtctcctcaggtgccatcgctgtgggcatgaagagagtcaacatgaagcggaaaccaaaaaagttacagcaagtgcaggcattggctgctataggacaaggccgtttgataggactttgggacgaccttttccgtcagttgaatcagcctattgcgcagattttactgactagaacggatttggtcgattacacccagtttaagaacgctgaaaatacattggaacagcttattaaaatgggtattattcctattgtcaatgagaatgacaccctatccattcaagaaatcaaatttggtgacaatgacaccttatccgccataacagctggtatgtgtcatgcagactacctgtttttggtgactgatgtggactgtctttacacggataaccctcgtacgaatccggacgctgagccaatcgtgttagttagaaatatgaggaatctaaacgtcaataccgaaagtggaggttccgccgtaggaacaggaggaatgacaactaaattgatcgcagctgatttgggtgtatctgcaggtgttacaacgattatttgcaaaagtgaacatcccgagcagattttggacattgtagagtacagtatccgtgctgatagagtcgaaaatgaggctaaatatctggtcatcaacgaagaggaaactgtggaacaatttcaagagatcaatcggtcagaactgagggagttgaacaagctggacattcctttgcatacacgtttcgttggccacagttttaatgctgttaataacaaagagttttggttactccatggactaaaggccaacggagccattatcattgatccaggttgttataaggctatcactagaaaaaacaaagctggtattcttccagctggaattatttccgtagagggtaatttccatgaatacgagtgtgttgatgttaaggtaggactaagagatccagatgacccacattcactagaccccaatgaagaactttacgtcgttggccgtgcccgttgtaattaccccagcaatcaaatcaacaaaattaagggtctacaaagctcgcagatcgagcaggttctaggttacgctgacggtgagtatgttgttcacagggacaacttggctttcccagtatttgccgatccagaactgttggatgttgttgagagtaccctgtctgaacaggagagagaatccaaaccaaataaatag

SEQ ID NO: 120 - DNA, 1425 bp, Artificial Sequence
  Sequence of the 3′-region that was used to knock into the PpPRO1 locus
aatttcacatatgctgcttgattatgtaattataccttgcgttcgatggcatcgatttcctcttctgtcaatcgcgcatcgcattaaaagtatacttttttttttttcctatagtactattcgccttattataaactttgctagtatgagttctacccccaagaaagagcctgatttgactcctaagaagagtcagcctccaaagaatagtctcggtgggggtaaaggctttagtgaggagggtttctcccaaggggacttcagcgctaagcatatactaaatcgtcgccctaacaccgaaggctcttctgtggcttcgaacgtcatcagttcgtcatcattgcaaaggttaccatcctctggatctggaagcgttgctgtgggaagtgtgttgggatcttcgccattaactctttctggagggttccacgggcttgatccaaccaagaataaaatagacgttccaaagtcgaaacagtcaaggagacaaagtgttctttctgacatgatttccacttctcatgcagctagaaatgatcactcagagcagcagttacaaactggacaacaatcagaacaaaaagaagaagatggtagtcgatcttctttttctgtttcttcccccgcaagagatatccggcacccagatgtactgaaaactgtcgagaaacatcttgccaatgacagcgagatcgactcatctttacaacttcaaggtggagatgtcactagaggcatttatcaatgggtaactggagaaagtagtcaaaaagataacccgcctttgaaacgagcaaatagttttaatgatttttcttctgtgcatggtgacgaggtaggcaaggcagatgctgaccacgatcgtgaaagcgtattcgacgaggatgatatctccattgatgatatcaaagttccgggagggatgcgtcgaagttttttattacaaaagcatagagaccaacaactttctggactgaataaaacggctcaccaaccaaaacaacttactaaacctaatttcttcacgaacaactttatagagtttttggcattgtatgggcattttgcaggtgaagatttggaggaagacgaagatgaagatttagacagtggttccgaatcagtcgcagtcagtgatagtgagggagaattcagtgaggctgacaacaatttgttgtatgatgaagagtctctcctattagcacctagtacctccaactatgcgagatcaagaataggaagtattcgtactcctacttatggatctttcagttcaaatgttggttcttcgtctattcatcagcagttaatgaaaagtcaaatcccgaagctgaagaaacgtggacagcacaagcataaaacacaatcaaaaatacgctcgaagaagcaaactaccaccgtaaaagcagtgttgctgctattaaa

SEQ ID NO: 121 - DNA, 2577 bp, Artificial Sequence
  DNA encoding Leishmania major STT3D
atgggtaaaagaaagggaaactccttgggagattctggttctgctgctactgcttccagagaggcttctgctcaagctgaagatgctgcttcccagactaagactgcttctccacctgctaaggttatcttgttgccaaagactttgactgacgagaaggacttcatcggtatcttcccatttccattctggccagttcacttcgttttgactgttgttgctttgttcgttttggctgcttcctgtttccaggctttcactgttagaatgatctccgttcaaatctacggttacttgatccacgaatttgacccatggttcaactacagagctgctgagtacatgtctactcacggatggagtgcttttttctcctggttcgattacatgtcctggtatccattgggtagaccagttggttctactacttacccaggattgcagttgactgctgttgctatccatagagctttggctgctgctggaatgccaatgtccttgaacaatgtttgtgttttgatgccagcttggtttggtgctatcgctactgctactttggctttctgtacttacgaggcttctggttctactgttgctgctgctgcagctgctttgtccttctccattatccctgctcacttgatgagatccatggctggtgagttcgacaacgagtgtattgctgttgctgctatgttgttgactttctactgttgggttcgttccttgagaactagatcctcctggccaatcggtgttttgacaggtgttgcttacggttacatggctgctgcttggggaggttacatcttcgttttgaacatggttgctatgcacgctggtatctcttctatggttgactgggctagaaacacttacaacccatccttgttgagagcttacactttgttctacgttgttggtactgctatcgctgtttgtgttccaccagttggaatgtctccattcaagtccttggagcagttgggagctttgttggttttggttttcttgtgtggattgcaagtttgtgaggttttgagagctagagctggtgttgaagttagatccagagctaatttcaagatcagagttagagttttctccgttatggctggtgttgctgctttggctatctctgttttggctccaactggttactttggtccattgtctgttagagttagagctttgtttgttgagcacactagaactggtaacccattggttgactccgttgctgaacatcaaccagcttctccagaggctatgtgggctttcttgcatgtttgtggtgttacttggggattgggttccattgttttggctgtttccactttcgttcactactccccatctaaggttttctggttgttgaactccggtgctgtttactacttctccactagaatggctagattgttgttgttgtccggtccagctgcttgtttgtccactggtatcttcgttggtactatcttggaggctgctgttcaattgtctttctgggactccgatgctactaaggctaagaagcagcaaaagcaggctcaaagacaccaaagaggtgctggtaaaggttctggtagagatgacgctaagaacgctactactgctagagctttctgtgacgttttcgctggttcttctttggcttggggtcacagaatggttttgtccattgctatgtgggctttggttactactactgctgtttccttcttctcctccgaatttgcttctcactccactaagttcgctgaacaatcctccaacccaatgatcgttttcgctgctgttgttcagaacagagctactggaaagccaatgaacttgttggttgacgactacttgaaggcttacgagtggttgagagactctactccagaggacgctagagttttggcttggtgggactacggttaccaaatcactggtatcggtaacagaacttccttggctgatggtaacacttggaaccacgagcacattgctactatcggaaagatgttgacttccccagttgttgaagctcactcccttgttagacacatggctgactacgttttgatttgggctggtcaatctggtgacttgatgaagtctccacacatggctagaatcggtaactctgtttaccacgacatttgtccagatgacccattgtgtcagcaattcggtttccacagaaacgattactccagaccaactccaatgatgagagcttccttgttgtacaacttgcacgaggctggaaaaagaaagggtgttaaggttaacccatctttgttccaagaggtttactcctccaagtacggacttgttagaatcttcaaggttatgaacgtttccgctgagtctaagaagtgggttgcagacccagctaacagagtttgtcacccacctggttcttggatttgtcctggtcaatacccacctgctaaagaaatccaagagatgttggctcacagagttccattcgaccaggttacaaacgctgacagaaagaacaatgttggttcctaccaagaggaatacatgagaagaatgagagagtccgagaacagaagataatag

SEQ ID NO: 122 - DNA, 375 bp, Artificial Sequence
  DNA encoding Sequence of the Sh ble ORF (Zeocin resistance marker)
atggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactga

SEQ ID NO: 123 - DNA, 427 bp, Artificial Sequence
  ScTEF1 promoter
gatcccccacacaccatagcttcaaaatgtttctactccttttttactcttccagattttctcggactccgcgcatcgccgtaccacttcaaaacacccaagcacagcatactaaatttcccctctttcttcctctagggtgtcgttaattacccgtactaaaggtttggaaaagaaaaaagagaccgcctcgtttctttttcttcgtcgaaaaaggcaataaaaatttttatcacgtttctttttcttgaaaatttttttttttgatttttttctctttcgatgacctcccattgatatttaagttaataaacggtcttcaatttctcaagtttcagtttcatttttcttgttctattacaactttttttacttcttgctcattagaaagaaagcatagcaatctaatctaagttttaattacaaa

SEQ ID NO: 124 - DNA, 2617 bp, Artificial Sequence
  PpAOX1 5′ flanking region
ggcttggccataattttgacattcgagtcatcaaaggtaaattcaaccggagacttgtattctttattgataactttctcatataggacattgtcaggaacacgatgaaaccaggatgcccccaaatccaatgagactgaggtttcatgagtcgcaaccaacctacctccaatacggtccctaccctctaaaatcaacgcattcacgccattgcttttgagatcgactgcagctttgatgcctgaaatcccagcgcctacaatgatgacatttggatttggttgactcatgttggtattgtgaaatagacgcagatcgggaacactgaaaaataacagttattattcgagatctaacatccaaagacgaaaggttgaatgaaacctttttgccatccgacatccacaggtccattctcacacataagtgccaaacgcaacaggaggggatacactagcagcagaccgttgcaaacgcaggacctccactcctcttctcctcaacacccacttttgccatcgaaaaaccagcccagttattgggcttgattggagctcgctcattccaattccttctattaggctactaacaccatgactttattagcctgtctatcctggcccccctggcgaggttcatgtttgtttatttccgaatgcaacaagctccgcattacacccgaacatcactccagatgagggctttctgagtgtggggtcaaatagtttcatgttccccaaatggcccaaaactgacagtttaaacgctgtcttggaacctaatatgacaaaagcgtgatctcatccaagatgaactaagtttggttcgttgaaatgctaacggccagttggtcaaaaagaaacttccaaaagtcggcataccgtttgtcttgtttggtattgattgacgaatgctcaaaaataatctcattaatgcttagcgcagtctctctatcgcttctgaaccccggtgcacctgtgccgaaacgcaaatggggaaacacccgctttttggatgattatgcattgtctccacattgtatgcttccaagattctggtgggaatactgctgatagcctaacgttcatgatcaaaatttaactgttctaacccctacttgacagcaatatataaacagaaggaagctgccctgtcttaaacctttttttttatcatcattattagcttactttcataattgcgactggttccaattgacaagcttttgattttaacgacttttaacgacaacttgagaagatcaaaaaacaactaattattcgaaacgatggctatccccgaagagtttcttggccataattttgacattcgagtcatcaaaggtaaattcaaccggagacttgtattctttattgataactttctcatataggacattgtcaggaacacgatgaaaccaggatgcccccaaatccaatgagactgaggtttcatgagtcgcaaccaacctacctccaatacggtccctaccctctaaaatcaacgcattcacgccattgcttttgagatcgactgcagctttgatgcctgaaatcccagcgcctacaatgatgacatttggatttggttgactcatgttggtattgtgaaatagacgcagatcgggaacactgaaaaataacagttattattcgagatctaacatccaaagacgaaaggttgaatgaaacctttttgccatccgacatccacaggtccattctcacacataagtgccaaacgcaacaggaggggatacactagcagcagaccgttgcaaacgcaggacctccactcctcttctcctcaacacccacttttgccatcgaaaaaccagcccagttattgggcttgattggagctcgctcattccaattcsttctattaggctactaacaccatgactttattagcctgtctatcctggcccccctggcgaggttcatgtttgtttatttccgaatgcaacaagctccgcattacacccgaacatcactccagatgagggctttctgagtgtggggtcaaatagtttcatgttccccaaatggcccaaaactgacagtttaaacgctgtcttggaacctaatatgacaaaagcgtgatctcatccaagatgaactaagtttggwtcgttgaaatgctaacggccagttggtcaaaaagaamcttccaaargtcggcataccgtttgtcttgtktggtattgattgacgaatgctcaaawataayctcattaatscttagcssatsyctctctatygcttctgaaccccggtgcacctgtgccgaaacgcaaatggggaaacacccgctttttggatgattatgcattgtctccacattgtatgcttccaagattctggtgggaatactgctgatagcctaacgttcatgatcaaaatttaactgttctaacccctacttgacagcaatatataaacagaaggaagctgccctgtcttaaacctttttttttatcatcattattagcttactttcataattgcgactggttccaattgacaagcttttgattttaacgacttttaacgacaacttgagaagatcaaaaaacaactaattattcgaaacgatggctatccccgaagagttt

SEQ ID NO: 125 - DNA, 2845 bp, Artificial Sequence
  PpAOX1 3′ flanking region
tcaagaggatgtcagaatgccatttgcctgagagatgcaggcttcatttttgatacttttttatttgtaacctatatagtataggattttttttgtcattttgtttcttctcgtacgagcttgctcctgatcagcctatctcgcagctgatgaatatcttgtggtaggggtttgggaaaatcattcgagtttgatgtttttcttggtatttcccactcctcttcagagtacagaagattaagtgagacgttcgtttgtgcaagcttcaacgatgccaaaagggtataataagcgtcatttgcagcattgtgaagaaaactatgtggcaagccaagcctgcgaagaatgtattttaagtttgactttgatgtattcacttgattaagccataattctcgagtatctatgattggaagtatgggaatggtgatacccgcattcttcagtgtcttgaggtctcctatcagattatgcccaactaaagcaaccggaggaggagatttcatggtaaatttctctgacttttggtcatcagtagactcgaactgtgagactatctcggttatgacagcagaaatgtccttcttggagacagtaaatgaagtcccaccaataaagaaatccttgttatcaggaacaaacttcttgtttcgaactttttcggtgccttgaactataaaatgtagagtggatatgtcgggtaggaatggagcgggcaaatgcttaccttctggaccttcaagaggtatgtagggtttgtagatactgatgccaacttcagtgacaacgttgctatttcgttcaaaccattccgaatccagagaaatcaaagttgtttgtctactattgatccaagccagtgcggtcttgaaactgacaatagtgtgctcgtgttttgaggtcatctttgtatgaataaatctagtctttgatctaaataatcttgacgagccagacgataataccaatctaaactctttaaacgttaaaggacaagtatgtctgcctgtattaaaccccaaatcagctcgtagtctgatcctcatcaacttgaggggcactatcttgttttagagaaatttgcggagatgcgatatcgagaaaaaggtacgctgattttaaacgtgaaatttatctcaagatctatgtacattagggcaaaacagctaatctatttggttctagtaagaacactgttagtcacaaattctaataccgaacgggctccactttcgggaagcgttcgtaaagcttcaagtgcttgatctctatatttactggccaacacacgagtcttctcaaccccgtcattctttataacggccgttttggcagtctcaacatcaccaggctttgagaaattacgtgctatcagaggtccgagactggggtcatttttccaagcatagagaattcaagaggatgtcagaatgccatttgcctgagagatgcaggcttcatttttgatacttttttatttgtaacctatatagtataggattttttttgtcattttgtttcttctcgtacgagcttgctcctgattagcctatctcgcagctgatgaatatcttgtggtaggggtttgggaaaatcattcgagtttgatgtttttcttggtatttcccactcctcttcagagtacagaagattaagtgagacgttcgtttgtgcaagcttcaacgatgccaaaagggtataataagcgtcatttgcagcattgtgaagaaaactatgtggcaagccaagcctgcgaagaatgtattttaagtttgactttgatgtattcacttgattaagccataattctcgagtatctatgattggaagtatgggaatggtgatacccgcattcttcagtgtcttgaggtctcctatcagattatgcccaactaaagcaaccggaggaggagatttcatggtaaatttctctgacttttggtcatcagtagactcgaactgtgagactatctcggttatgacagcagaaatgtccttcttggagacagtaaatgaagtcccaccaataaagaaatccttgttatcaggaacaaacttcttgtttcgaactttttcggtgccttgaactataaaatgtagagtggatatgtcgggtaggaatgggagcgggcaaatgcttaccttcttgacccttcaagaggtatgtagggtttgtagatactgatgccaactttcagtgacaacgttgctatttcgttcaaacccattccgaatccagagaaatcaaagtttgtttgtctactattgatccaagccagtgcggtcttgaaaactgacaatagtgtgctcgtgttttgaggtcatcttttgtatgaataaatctagtcttttgatctaaataatcttgacgagccagacgataataccaatctaaactctttaaacgttaaaggacaagtatgtctgcctgtattaaaccccaaatcagctcgtagtctgatcctcatcaacttgaggggcactatcttgttttagagaaatttgcggagatgcgatatcgagaaaaaggtacgctgattttaaacgtgaaatttatctcaagatctatgtacattagggcaaaacagctaatctatttggttctagtaagaacactgttagtcacaaattctaataccgaacgggctccactttcgggaagcgttcgtaaagcttcaagtgcttgatctctatatttactggccaacacacgagtcttctcaaccccgtcattctttataacggccgttttggcagtctcaacatcaccaggctttgagaaattacgtgctatcagaggtccgagactggggtcatttttccaagcatagagaatggccgctgt

SEQ ID NO: 126 - DNA, 447 bp, Artificial Sequence
  DNA encoding Pre-proinsulin analogue precursor S.c. alpha mating factor signal sequence and pro-peptide + N- terminal spacer + B chain des(B30) + C-peptide “AAK”+ A chain
atgagatttccttcaatttttactgcagttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctctcgagaaaagggaagaggcagaagctgaggccgaaccaaagtttgttaaccaacatttgtgtggttcacaccttgttgaggctttgtaccttgtctgcggtgaaagaggatttttctatactcctaaggctgccaaaggaattgtcgagcaatgttgcacatctatctgttccttgtaccagcttgaaaactattgcaattaa

SEQ ID NO: 127 - protein, 148 aa, Artificial Sequence
  Pre-proinsulin analogue precursor  S.c. alpha mating factor signal sequence and pro-peptide + B chain des(B30) + C-peptide “AAK”+ A chain
MRFPSIFTAVLFAASSALAAPVNTTTEDETAQIPAEAVIGYSDLEGDFDVAVLPFSNSTNNGLLFINTTIASIAAKEEGVSLEKREEAEAEAEPKFVNQHLCGSHLVEALYLVCGERGFFYTPKAAKGIVEQCCTSICSLYQLENYCN

SEQ ID NO: 128 - DNA, 456 bp, Artificial Sequence
  DNA encoding Pre-proinsulin analogue precursor S.c. alpha mating factor signal sequence and pro-peptide + N- terminal spacer + B chain NTT(-2) des(B30) + C-peptide “AAK” + A chain
atgagatttccttcaatttttactgcagttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctctcgagaaaagggaagaggcagaagctgaggccgaaccaaagaacactacattcgttaaccaacatttgtgtggttcacaccttgttgaggctttgtaccttgtctgcggtgaaagaggatttttctatacccctaaggctgccaaaggaattgtcgagcaatgttgcacttctatctgttccttgtaccagcttgaaaactattgcaattaa

SEQ ID NO: 129 - protein, 151 aa, Artificial Sequence
  Pre-proinsulin analogue precursor  S.c. alpha mating factor signal sequence and pro-peptide + N-terminal spacer + B chain NTT(-2) des(B30) + C-peptide “AAK” + A chain
MRFPSIFTAVLFAASSALAAPVNTTTEDETAQIPAEAVIGYSDLEGDFDVAVLPFSNSTNNGLLFINTTIASIAAKEEGVSLEKREEAEAEAEPKNTTFVNQHLCGSHLVEALYLVCGERGFFYTPKAAKGIVEQCCTSICSLYQLENYCN

SEQ ID NO: 130 - DNA, 456 bp, Artificial Sequence
  DNA encoding Pre-proinsulin analogue precursor S.c. alpha mating factor signal sequence and pro-peptide + N- terminal spacer + B chain NGT(-2) des(B30) + C-peptide “AAK” + A chain
atgagatttccttcaatttttactgcagttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctctcgagaaaagggaagaggcagaagctgaggccgaaccaaagaacggtactttcgttaaccaacatttgtgtggatcacaccttgttgaggctttgtaccttgtctgcggtgaaagaggatttttctatactcctaaggctgccaaaggtattgtcgagcaatgttgcacatctatctgttccttgtaccagcttgaaaactattgcaattaa

SEQ ID NO: 131 - protein, 151 aa, Artificial Sequence
  Pre-proinsulin analogue precursor  S.c. alpha mating factor signal sequence and pro-peptide + N-terminal spacer + B chain NGT(-2) des(B30) + C-peptide “AAK” + A chain
MRFPSIFTAVLFAASSALAAPVNTTTEDETAQIPAEAVIGYSDLEGDFDVAVLPFSNSTNNGLLFINTTIASIAAKEEGVSLEKREEAEAEAEPKNGTFVNQHLCGSHLVEALYLVCGERGFFYTPKAAKGIVEQCCTSICSLYQLENYCN

SEQ ID NO: 132 - DNA, 456 bp, Artificial Sequence
  DNA encoding Pre-proinsulin analogue precursor S.c. alpha mating factor signal sequence and pro-peptide + N- terminal spacer + B chain des(B30) + C-peptide “AAK” + A chain NTT(-2)
atgagatttccttcaatttttactgcagttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctctcgagaaaagggaagaggcagaagctgaggccgaaccaaagtttgttaaccaacatttgtgtggttcacaccttgttgaggctttgtaccttgtctgcggtgaaagaggatttttctatacccctaaggctgccaaaaatactacaggaattgtcgagcaatgttgcacttctatctgttccttgtaccagcttgaaaactattgcaattaa

SEQ ID NO: 133 - protein, 151 aa, Artificial Sequence
  Pre-proinsulin analogue   S.c. alpha mating factor signal sequence and pro-peptide + N-terminal spacer + B chain des(B30) + C-peptide “AAK”+ A chain NTT(-2)
MRFPSIFTAVLFAASSALAAPVNTTTEDETAQIPAEAVIGYSDLEGDFDVAVLPFSNSTNNGLLFINTTIASIAAKEEGVSLEKREEAEAEAEPKFVNQHLCGSHLVEALYLVCGERGFFYTPKAAKNTTGIVEQCCTSICSLYQLENYCN

SEQ ID NO: 134 - DNA, 450 bp, Artificial Sequence
  DNA encoding Pre-proinsulin analogue precursor S.c. alpha mating factor signal sequence and pro-peptide + N- terminal spacer + B chain P28N + C-peptide “AAK” + A chain
atgagatttccttcaatttttactgcagttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctctcgagaaaagggaagaggcagaagctgaggccgaaccaaagtttgttaaccaacatttgtgtggttcacaccttgttgaggctttgtaccttgtctgcggtgaaagaggatttttctatactaataagacagctgccaaaggaattgtcgagcaatgttgcacttctatctgttccttgtaccagcttgaaaactattgcaattaa

SEQ ID NO: 135 - protein, 149 aa, Artificial Sequence
  Pre-proinsulin analogue precursor  S.c. alpha mating factor signal sequence and pro-peptide + N-terminal spacer + B chain P28N + C-peptide “AAK” + A chain
MRFPSIFTAVLFAASSALAAPVNTTTEDETAQIPAEAVIGYSDLEGDFDVAVLPFSNSTNNGLLFINTTIASIAAKEEGVSLEKREEAEAEAEPKFVNQHLCGSHLVEALYLVCGERGFFYTNKTAAKGIVEQCCTSICSLYQLENYCN

SEQ ID NO: 136 - DNA, 459 bp, Artificial Sequence
  DNA encoding Pre-proinsulin analogue precursor S.c. alpha mating factor signal sequence and pro-peptide + N- terminal spacer + B chain NTT(-2) P28N + C-peptide “AAK” + A chain
atgagatttccttcaatttttactgcagttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctctcgagaaaagggaagaggcagaagctgaggccgaaccaaagaacactacattcgttaaccaacatttgtgtggttcacaccttgttgaggctttgtaccttgtctgcggtgaaagaggatttttctataccaacaagactgctgccaaaggaattgtcgagcaatgttgcacatctatctgttccttgtaccagcttgaaaactattgcaattaa

SEQ ID NO: 137 - protein, 152 aa, Artificial Sequence
  Pre-proinsulin analogue precursor  S.c. alpha mating factor signal sequence and pro-peptide + N-terminal spacer + B chain NTT(-2) P28N + C-peptide “AAK” + A chain
MRFPSIFTAVLFAASSALAAPVNTTTEDETAQIPAEAVIGYSDLEGDFDVAVLPFSNSTNNGLLFINTTIASIAAKEEGVSLEKREEAEAEAEPKNTTFVNQHLCGSHLVEALYLVCGERGFFYTNKTAAKGIVEQCCTSICSLYQLENYCN

SEQ ID NO: 138 - DNA, 459 bp, Artificial Sequence
  DNA encoding Pre-proinsulin analogue precursor S.c. alpha mating factor signal sequence and pro-peptide + N- terminal spacer + B chain NGT(-2) P28N + C-peptide “AAK” + A chain
atgagatttccttcaatttttactgcagttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctctcgagaaaagggaagaggcagaagctgaggccgaaccaaagaacggtacctttgttaatcaacatttgtgtggatcacaccttgttgaggctttgtaccttgtctgcggtgaaagaggatttttctatactaacaagacagctgccaaaggtattgtcgagcaatgttgcacttctatctgttccttgtaccagcttgaaaactattgcaattaa

SEQ ID NO: 139 - protein, 152 aa, Artificial Sequence
  Pre-proinsulin analogue precursor  S.c. alpha mating factor signal sequence and pro-peptide + N-terminal spacer + B chain NGT(-2) P28N + C-peptide “AAK” + A chain
MRFPSIFTAVLFAASSALAAPVNTTTEDETAQIPAEAVIGYSDLEGDFDVAVLPFSNSTNNGLLFINTTIASIAAKEEGVSLEKREEAEAEAEPKNGTFVNQHLCGSHLVEALYLVCGERGFFYTNKTAAKGIVEQCCTSICSLYQLENYCN

SEQ ID NO: 140 - DNA, 459 bp, Artificial Sequence
  DNA encoding Pre-proinsulin analogue precursor S.c. alpha mating factor signal sequence and pro-peptide + N- terminal spacer + B chain P28N + C-peptide “AAK” + A chain NTT(-2)
atgagatttccttcaatttttactgcagttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctctcgagaaaagggaagaggcagaagctgaggccgaaccaaagtttgttaaccaacatttgtgtggttcacaccttgttgaggctttgtaccttgtctgcggtgaaagaggatttttctataccaacaagactgctgccaaaaatactacaggaattgtcgagcaatgttgcacatctatctgttccttgtaccagcttgaaaactattgcaattaa

SEQ ID NO: 141 - protein, 152 aa, Artificial Sequence
  Pre-proinsulin analogue precursor  S.c. alpha mating factor signal sequence and pro-peptide + N-terminal spacer + B chain P28N + C-peptide “AAK” + A chain NTT(-2)
MRFPSIFTAVLFAASSALAAPVNTTTEDETAQIPAEAVIGYSDLEGDFDVAVLPFSNSTNNGLLFINTTIASIAAKEEGVSLEKREEAEAEAEPKFVNQHLCGSHLVEALYLVCGERGFFYTNKTAAKNTTGIVEQCCTSICSLYQLENYCN

SEQ ID NO: 142 - DNA, 447 bp, Artificial Sequence
  DNA encoding Pre-proinsulin analogue precursor S.c. alpha mating factor signal sequence and pro-peptide + N- terminal spacer + B chain P28N des(B30) + C-peptide “AAK” + A chain
atgagatttccttcaatttttactgcagttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctctcgagaaaagggaagaggcagaagctgaggccgaaccaaagtttgttaaccaacatttgtgtggttcacaccttgttgaggctttgtaccttgtctgcggtgaaagaggatttttctatactaataaggctgccaaaggaattgtcgagcaatgttgcacatctatctgttccttgtaccagcttgaaaactattgcaattaa

SEQ ID NO: 143 - protein, 148 aa, Artificial Sequence
  Pre-proinsulin analogue precursor  S.c. alpha mating factor signal sequence and pro-peptide + B chain P28N des(B30) + C-peptide “AAK” + A chain
MRFPSIFTAVLFAASSALAAPVNTTTEDETAQIPAEAVIGYSDLEGDFDVAVLPFSNSTNNGLLFINTTIASIAAKEEGVSLEKREEAEAEAEPKFVNQHLCGSHLVEALYLVCGERGFFYTNKAAKGIVEQCCTSICSLYQLENYCN

SEQ ID NO: 144 - DNA, 465 bp, Artificial Sequence
  DNA encoding Pre-proinsulin analogue precursor S.c. alpha mating factor signal sequence and pro-peptide + N- terminal spacer + B chain NGT(-2) des(B30) + C-peptide “AAK” + A chain NGT(-2)
atgagatttccttcaatttttactgcagttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctctcgagaaaagggaagaggcagaagctgaggccgaaccaaagaacggtactttcgttaaccaacatttgtgtggatcacaccttgttgaggctttgtaccttgtctgcggtgaaagaggatttttctatactcctaaggctgccaaaaacggtacaggaattgtcgagcaatgttgcacctctatctgttccttgtaccagcttgaaaactattgcaattaa

SEQ ID NO: 145 - protein, 154 aa, Artificial Sequence
  Pre-proinsulin analogue precursor  S.c. alpha mating factor signal sequence and pro-peptide + N-terminal spacer + B chain NGT(-2) des(B30) + C-peptide “AAK” + A chain NGT(-2)
MRFPSIFTAVLFAASSALAAPVNTTTEDETAQIPAEAVIGYSDLEGDFDVAVLPFSNSTNNGLLFINTTIASIAAKEEGVSLEKREEAEAEAEPKNGTFVNQHLCGSHLVEALYLVCGERGFFYTPKAAKNGTGIVEQCCTSICSLYQLENYCN

SEQ ID NO: 146 - DNA, 468 bp, Artificial Sequence
  DNA encoding Pre-proinsulin analogue precursor S.c. alpha mating factor signal sequence and pro-peptide + N- terminal spacer + B chain NGT(-2) P28N + C-peptide “AAK” + A chain NGT(-2)
atgagatttccttcaatttttactgcagttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctctcgagaaaagggaagaggcagaagctgaggccgaaccaaagaacggtacattcgttaaccaacatttgtgtggatcacaccttgttgaggctttgtaccttgtctgcggtgaaagaggatttttctatactaacaagacagctgccaaaaatggtaccggaattgtcgagcaatgttgcacttctatctgttccttgtaccagcttgaaaactattgcaattaa

SEQ ID NO: 147 - protein, 155 aa, Artificial Sequence
  Pre-proinsulin analogue precursor  S.c. alpha mating factor signal sequence and pro-peptide + N-terminal spacer + B chain NGT(-2) P28N + C-peptide “AAK” + A chain NGT(-2)
MRFPSIFTAVLFAASSALAAPVNTTTEDETAQIPAEAVIGYSDLEGDFDVAVLPFSNSTNNGLLFINTTIASIAAKEEGVSLEKREEAEAEAEPKNGTFVNQHLCGSHLVEALYLVCGERGFFYTNKTAAKNGTGIVEQCCTSICSLYQLENYCN

SEQ ID NO: 148 - protein, 85 aa, Artificial Sequence
  Sc alpha mating factor signal sequence and pro-peptide
MRFPSIFTAVLFAASSALAAPVNTTTEDETAQIPAEAVIGYSDLEGDFDVAVLPFSNSTNNGLLFINTTIASIAAKEEGVSLEKR

SEQ ID NO: 149 - protein, 10 aa, Artificial Sequence
  N-terminal spacer
EEAEAEAEPK

SEQ ID NO: 150 - protein, 63 aa, Artificial Sequence
  Proinsulin (des(B30)) analogue precursor with N-terminal spacer and C-peptide “AAK”
EEAEAEAEPKFVNQHLCGSHLVEALYLVCGERGFFYTPKAAKGIVEQCCTSICSLYQLENYCN

SEQ ID NO: 151 - protein, 66 aa, Artificial Sequence
  Proinsulin (BNTT(-2) des(B30)) analogue precursor with N-terminal spacer and C-peptide “AAK”
EEAEAEAEPKNTTFVNQHLCGSHLVEALYLVCGERGFFYTPKAAKGIVEQCCTSICSLYQLENYCN

SEQ ID NO: 152 - protein, 66 aa, Artificial Sequence
  Proinsulin (BNGT(-2) des(B30)) analogue precursor with N-terminal spacer and C-peptide “AAK”
EEAEAEAEPKNGTFVNQHLCGSHLVEALYLVCGERGFFYTPKAAKGIVEQCCTSICSLYQLENYCN

SEQ ID NO: 153 - protein, 66 aa, Artificial Sequence
  Proinsulin (des(B30) ANTT(-2)) analogue precursor with N-terminal spacer and C-peptide “AAK”
EEAEAEAEPKFVNQHLCGSHLVEALYLVCGERGFFYTPKAAKNTTGIVEQCCTSICSLYQLENYCN

SEQ ID NO: 154 - protein, 64 aa, Artificial Sequence
  Proinsulin (BP28N) analogue precursor with N- terminal spacer and C-peptide “AAK”
EEAEAEAEPKFVNQHLCGSHLVEALYLVCGERGFFYTNKTAAKGIVEQCCTSICSLYQLENYCN

SEQ ID NO: 155 - protein, 67 aa, Artificial Sequence
  Proinsulin (BNTT(-2) BP28N) analogue precursor with N-terminal spacer and C-peptide “AAK”
EEAEAEAEPKNTTFVNQHLCGSHLVEALYLVCGERGFFYTNKTAAKGIVEQCCTSICSLYQLENYCN

SEQ ID NO: 156 - protein, 67 aa, Artificial Sequence
  Proinsulin (BNGT(-2) BP28N) analogue precursor with N-terminal spacer and C-peptide “AAK”
EEAEAEAEPKNGTFVNQHLCGSHLVEALYLVCGERGFFYTNKTAAKGIVEQCCTSICSLYQLENYCN

SEQ ID NO: 157 - protein, 67 aa, Artificial Sequence
  Proinsulin (BP28N ANTT(-2)) analogue precursor with N-terminal spacer and C-peptide “AAK”
EEAEAEAEPKFVNQHLCGSHLVEALYLVCGERGFFYTNKTAAKNTTGIVEQCCTSICSLYQLENYCN

SEQ ID NO: 158 - protein, 63 aa, Artificial Sequence
  Proinsulin (BP28N des(B30)) analogue precursor with N-terminal spacer and C-peptide “AAK”
EEAEAEAEPKFVNQHLCGSHLVEALYLVCGERGFFYTNKAAKGIVEQCCTSICSLYQLENYCN

SEQ ID NO: 159 - protein, 69 aa, Artificial Sequence
  Proinsulin (BNGT(-2) des(B30) ANGT(-2)) analogue precursor with N-terminal spacer and C-peptide “AAK”
EEAEAEAEPKNGTFVNQHLCGSHLVEALYLVCGERGFFYTPKAAKNGTGIVEQCCTSICSLYQLENYCN

SEQ ID NO: 160 - protein, 70 aa, Artificial Sequence
  Proinsulin (BNGT(-2) BP28N ANGT(-2)) analogue precursor with N-terminal spacer and C-peptide “AAK”
EEAEAEAEPKNGTFVNQHLCGSHLVEALYLVCGERGFFYTNKTAAKNGTGIVEQCCTSICSLYQLENYCN

SEQ ID NO: 161 - protein, 21 aa, Artificial Sequence
  B-chain peptide core sequence
HLCGSHLVEALYLVCGERGFF

SEQ ID NO: 162 - protein, 21 aa, Artificial Sequence
  A-chain analog
GIVEQCCNSXCSLYQLENYCN

SEQ ID NO: 163 - protein, 21 aa, Artificial Sequence
  A-chain analog
GIVEQCCTSICSLYQLENYCN

SEQ ID NO: 164 - protein, 21 aa, Artificial Sequence
  A-chain analog
GIVEQCCTSNCSLYQLENYCN

SEQ ID NO: 165 - protein, 21 aa, Artificial Sequence
  A-chain analog
GIVEQCCNSXCSLYQLENYCN

SEQ ID NO: 166 - protein, 24 aa, Artificial Sequence
  A-chain analog
NXXGIVEQCCTSICSLYQLENYCN

SEQ ID NO: 167 - protein, 24 aa, Artificial Sequence
  A-chain analog
NXXGIVEQCCNSXCSLYQLENYCN

SEQ ID NO: 168 - protein, 24 aa, Artificial Sequence
  A-chain analog
NXXGIVEQCCTSNCSLYQLENYCN

SEQ ID NO: 169 - protein, 24 aa, Artificial Sequence
  A-chain analog
NXXGIVEQCCTSICSLYQLENYCN

SEQ ID NO: 170 - protein, 24 aa, Artificial Sequence
  A-chain analog
NXXGIVEQCCTSNCSLYQLENYCN

SEQ ID NO: 171 - protein, 24 aa, Artificial Sequence
  A-chain analog
NXXGIVEQCCNSXCSLYQLENYCN

SEQ ID NO: 172 - protein, 24 aa, Artificial Sequence
  A-chain analog
NXXGIVEQCCTSICSLYQLENYCG

SEQ ID NO: 173 - protein, 24 aa, Artificial Sequence
  A-chain analog
NXXGIVEQCCNSXCSLYQLENYCG

SEQ ID NO: 174 - protein, 24 aa, Artificial Sequence
  A-chain analog
NXXGIVEQCCTSNCSLYQLENYCG

SEQ ID NO: 175 - protein, 21 aa, Artificial Sequence
  A-chain analog
GIVEQCCNSXCSLYQLENYCG

SEQ ID NO: 176 - protein, 21 aa, Artificial Sequence
  A-chain analog
GIVEQCCTSNCSLYQLENYCG

SEQ ID NO: 177 - protein, 30 aa, Artificial Sequence
  B-chain analog
FVNQXLCGSHLVEALYLVCGERGFFYTPKT

SEQ ID NO: 178 - protein, 30 aa, Artificial Sequence
  B-chain analog
FVNQHLCGSHLVEALYLVCGERGFNYTNKT

SEQ ID NO: 179 - protein, 30 aa, Artificial Sequence
  B-chain analog
FVNQXLCGSHLVEALYLVCGERGFNYTPKT

SEQ ID NO: 180 - protein, 30 aa, Artificial Sequence
  B-chain analog
FVNQXLCGSHLVEALYLVCGERGFFYTNKT

SEQ ID NO: 181 - protein, 30 aa, Artificial Sequence
  B-chain analog
FVNQXLCGSHLVEALYLVCGERGFNYTNKT

SEQ ID NO: 182 - protein, 33 aa, Artificial Sequence
  B-chain analog
NXXFVNQHLCGSHLVEALYLVCGERGFFYTPKT

SEQ ID NO: 183 - protein, 33 aa, Artificial Sequence
  B-chain analog
NXXFVNQXLCGSHLVEALYLVCGERGFFYTPKT

SEQ ID NO: 184 - protein, 33 aa, Artificial Sequence
  B-chain analog
NXXFVNQHLCGSHLVEALYLVCGERGFNYTPKT

SEQ ID NO: 185 - protein, 33 aa, Artificial Sequence
  B-chain analog
NXXFVNQHLCGSHLVEALYLVCGERGFFYTNKT

SEQ ID NO: 186 - protein, 33 aa, Artificial Sequence
  B-chain analog
NXXFVNQHLCGSHLVEALYLVCGERGFNYTNKT

SEQ ID NO: 187 - protein, 33 aa, Artificial Sequence
  B-chain analog
NXXFVNQXLCGSHLVEALYLVCGERGFNYTPKT

SEQ ID NO: 188 - protein, 33 aa, Artificial Sequence
  B-chain analog
NXXFVNQXLCGSHLVEALYLVCGERGFFYTNKT

SEQ ID NO: 189 - protein, 33 aa, Artificial Sequence
  B-chain analog
NXXFVNQXLCGSHLVEALYLVCGERGFNYTNKT

SEQ ID NO: 190 - protein, 31 aa, Artificial Sequence
  B-chain analog
FVNQHLCGSHLVEALYLVCGERGFFYTPKTN

SEQ ID NO: 191 - protein, 31 aa, Artificial Sequence
  B-chain analog
FVNQXLCGSHLVEALYLVCGERGFFYTPKTN

SEQ ID NO: 192 - protein, 31 aa, Artificial Sequence
  B-chain analog
FVNQHLCGSHLVEALYLVCGERGFNYTPKTN

SEQ ID NO: 193 - protein, 31 aa, Artificial Sequence
  B-chain analog
FVNQHLCGSHLVEALYLVCGERGFFYTNKTN

SEQ ID NO: 194 - protein, 31 aa, Artificial Sequence
  B-chain analog
FVNQHLCGSHLVEALYLVCGERGFNYTNKTN

SEQ ID NO: 195 - protein, 31 aa, Artificial Sequence
  B-chain analog
FVNQXLCGSHLVEALYLVCGERGFNYTPKTN

SEQ ID NO: 196 - protein, 31 aa, Artificial Sequence
  B-chain analog
FVNQXLCGSHLVEALYLVCGERGFFYTNKTN

SEQ ID NO: 197 - protein, 31 aa, Artificial Sequence
  B-chain analog
FVNQXLCGSHLVEALYLVCGERGFNYTNKTN

SEQ ID NO: 198 - protein, 34 aa, Artificial Sequence
  B-chain analog
NXXFVNQHLCGSHLVEALYLVCGERGFFYTPKTN

SEQ ID NO: 199 - protein, 34 aa, Artificial Sequence
  B-chain analog
NXXFVNQXLCGSHLVEALYLVCGERGFFYTPKTN

SEQ ID NO: 200 - protein, 34 aa, Artificial Sequence
  B-chain analog
NXXFVNQHLCGSHLVEALYLVCGERGFNYTPKTN

SEQ ID NO: 201 - protein, 34 aa, Artificial Sequence
  B-chain analog
NXXFVNQHLCGSHLVEALYLVCGERGFFYTNKTN

SEQ ID NO: 202 - protein, 34 aa, Artificial Sequence
  B-chain analog
NXXFVNQHLCGSHLVEALYLVCGERGFNYTNKTN

SEQ ID NO: 203 - protein, 34 aa, Artificial Sequence
  B-chain analog
NXXFVNQXLCGSHLVEALYLVCGERGFNYTPKTN

SEQ ID NO: 204 - protein, 34 aa, Artificial Sequence
  B-chain analog
NXXFVNQXLCGSHLVEALYLVCGERGFFYTNKTN

SEQ ID NO: 205 - protein, 34 aa, Artificial Sequence
  B-chain analog
NXXFVNQXLCGSHLVEALYLVCGERGFNYTNKTN

SEQ ID NO: 206 - protein, 32 aa, Artificial Sequence
  B-chain analog
FVNQXLCGSHLVEALYLVCGERGFFYTPKTRR

SEQ ID NO: 207 - protein, 32 aa, Artificial Sequence
  B-chain analog
FVNQHLCGSHLVEALYLVCGERGFNYTPKTRR

SEQ ID NO: 208 - protein, 32 aa, Artificial Sequence
  B-chain analog
FVNQHLCGSHLVEALYLVCGERGFFYTNKTRR

SEQ ID NO: 209 - protein, 32 aa, Artificial Sequence
  B-chain analog
FVNQHLCGSHLVEALYLVCGERGFNYTNKTRR

SEQ ID NO: 210 - protein, 32 aa, Artificial Sequence
  B-chain analog
FVNQXLCGSHLVEALYLVCGERGFNYTPKTRR

SEQ ID NO: 211 - protein, 32 aa, Artificial Sequence
  B-chain analog
FVNQXLCGSHLVEALYLVCGERGFFYTNKTRR

SEQ ID NO: 212 - protein, 32 aa, Artificial Sequence
  B-chain analog
FVNQXLCGSHLVEALYLVCGERGFNYTNKTRR

SEQ ID NO: 213 - protein, 35 aa, Artificial Sequence
  B-chain analog
NXXFVNQHLCGSHLVEALYLVCGERGFFYTPKTRR

SEQ ID NO: 214 - protein, 35 aa, Artificial Sequence
  B-chain analog
NXXFVNQXLCGSHLVEALYLVCGERGFFYTPKTRR

SEQ ID NO: 215 - protein, 35 aa, Artificial Sequence
  B-chain analog
NXXFVNQHLCGSHLVEALYLVCGERGFNYTPKTRR

SEQ ID NO: 216 - protein, 35 aa, Artificial Sequence
  B-chain analog
NXXFVNQHLCGSHLVEALYLVCGERGFFYTNKTRR

SEQ ID NO: 217 - protein, 35 aa, Artificial Sequence
  B-chain analog
NXXFVNQHLCGSHLVEALYLVCGERGFNYTNKTRR

SEQ ID NO: 218 - protein, 35 aa, Artificial Sequence
  B-chain analog
NXXFVNQXLCGSHLVEALYLVCGERGFNYTPKTRR

SEQ ID NO: 219 - protein, 35 aa, Artificial Sequence
  B-chain analog
NXXFVNQXLCGSHLVEALYLVCGERGFFYTNKTRR

SEQ ID NO: 220 - protein, 35 aa, Artificial Sequence
  B-chain analog
NXXFVNQXLCGSHLVEALYLVCGERGFNYTNKTRR

SEQ ID NO: 221 - protein, 35 aa, Artificial Sequence
  B-chain analog
FVNQHLCGSHLVEALYLVCGERGFFYTPKTNXXRR

SEQ ID NO: 222 - protein, 35 aa, Artificial Sequence
  B-chain analog
FVNQXLCGSHLVEALYLVCGERGFFYTPKTNXXRR

SEQ ID NO: 223 - protein, 35 aa, Artificial Sequence
  B-chain analog
FVNQHLCGSHLVEALYLVCGERGFNYTPKTNXXRR

SEQ ID NO: 224 - protein, 35 aa, Artificial Sequence
  B-chain analog
FVNQHLCGSHLVEALYLVCGERGFFYTNKTNXXRR

SEQ ID NO: 225 - protein, 35 aa, Artificial Sequence
  B-chain analog
FVNQHLCGSHLVEALYLVCGERGFNYTNKTNXXRR

SEQ ID NO: 226 - protein, 35 aa, Artificial Sequence
  B-chain analog
FVNQXLCGSHLVEALYLVCGERGFNYTPKTNXXRR

SEQ ID NO: 227 - protein, 35 aa, Artificial Sequence
  B-chain analog
FVNQXLCGSHLVEALYLVCGERGFFYTNKTNXXRR

SEQ ID NO: 228 - protein, 35 aa, Artificial Sequence
  B-chain analog
FVNQXLCGSHLVEALYLVCGERGFNYTNKTNXXRR

SEQ ID NO: 229 - protein, 38 aa, Artificial Sequence
  B-chain analog
NXXFVNQHLCGSHLVEALYLVCGERGFFYTPKTNXXRR

SEQ ID NO: 230 - protein, 38 aa, Artificial Sequence
  B-chain analog
NXXFVNQXLCGSHLVEALYLVCGERGFFYTPKTNXXRR

SEQ ID NO: 231 - protein, 38 aa, Artificial Sequence
  B-chain analog
NXXFVNQHLCGSHLVEALYLVCGERGFNYTPKTNXXRR

SEQ ID NO: 232 - protein, 38 aa, Artificial Sequence
  B-chain analog
NXXFVNQHLCGSHLVEALYLVCGERGFFYTNKTNXXRR

SEQ ID NO: 233 - protein, 38 aa, Artificial Sequence
  B-chain analog
NXXFVNQHLCGSHLVEALYLVCGERGFNYTNKTNXXRR

SEQ ID NO: 234 - protein, 38 aa, Artificial Sequence
  B-chain analog
NXXFVNQXLCGSHLVEALYLVCGERGFNYTPKTNXXRR

SEQ ID NO: 235 - protein, 38 aa, Artificial Sequence
  B-chain analog
NXXFVNQXLCGSHLVEALYLVCGERGFFYTNKTNXXRR

SEQ ID NO: 236 - protein, 38 aa, Artificial Sequence
  B-chain analog
NXXFVNQXLCGSHLVEALYLVCGERGFNYTNKTNXXRR

SEQ ID NO: 237 - protein, 29 aa, Artificial Sequence
  B-chain analog
FVNQXLCGSHLVEALYLVCGERGFFYTPK

SEQ ID NO: 238 - protein, 29 aa, Artificial Sequence
  B-chain analog
FVNQHLCGSHLVEALYLVCGERGFNYTPK

SEQ ID NO: 239 - protein, 29 aa, Artificial Sequence
  B-chain analog
FVNQHLCGSHLVEALYLVCGERGFFYTNK

SEQ ID NO: 240 - protein, 29 aa, Artificial Sequence
  B-chain analog
FVNQHLCGSHLVEALYLVCGERGFNYTNK

SEQ ID NO: 241 - protein, 29 aa, Artificial Sequence
  B-chain analog
FVNQXLCGSHLVEALYLVCGERGFNYTPK

SEQ ID NO: 242 - protein, 29 aa, Artificial Sequence
  B-chain analog
FVNQXLCGSHLVEALYLVCGERGFFYTNK

SEQ ID NO: 243 - protein, 29 aa, Artificial Sequence
  B-chain analog
FVNQXLCGSHLVEALYLVCGERGFNYTNK

SEQ ID NO: 244 - protein, 32 aa, Artificial Sequence
  B-chain analog
NXXFVNQHLCGSHLVEALYLVCGERGFFYTPK

SEQ ID NO: 245 - protein, 32 aa, Artificial Sequence
  B-chain analog
NXXFVNQXLCGSHLVEALYLVCGERGFFYTPK

SEQ ID NO: 246 - protein, 32 aa, Artificial Sequence
  B-chain analog
NXXFVNQHLCGSHLVEALYLVCGERGFNYTPK

SEQ ID NO: 247 - protein, 32 aa, Artificial Sequence
  B-chain analog
NXXFVNQHLCGSHLVEALYLVCGERGFFYTNK

SEQ ID NO: 248 - protein, 32 aa, Artificial Sequence
  B-chain analog
NXXFVNQHLCGSHLVEALYLVCGERGFNYTNK

SEQ ID NO: 249 - protein, 32 aa, Artificial Sequence
  B-chain analog
NXXFVNQXLCGSHLVEALYLVCGERGFNYTPK

SEQ ID NO: 250 - protein, 32 aa, Artificial Sequence
  B-chain analog
NXXFVNQXLCGSHLVEALYLVCGERGFFYTNK

SEQ ID NO: 251 - protein, 32 aa, Artificial Sequence
  B-chain analog
NXXFVNQXLCGSHLVEALYLVCGERGFNYTNK

SEQ ID NO: 252 - protein, 21 aa, Artificial Sequence
  A-chain analog
GIVEQCCTSNCSLYQLENYCN

SEQ ID NO: 253 - protein, 30 aa, Artificial Sequence; identical to SEQ ID NO: 44
  B-chain analog

SEQ ID NO: 254 - protein, 30 aa, Artificial Sequence; identical to SEQ ID NO: 26
  B-chain analog

SEQ ID NO: 255 - DNA, 1215 bp, Artificial Sequence
  DNA encodes Saccharomyces cerevisiae ARR3
atgtcagaagatcaaaaaagtgaaaattccgtaccttctaaggttaatatggtgaatcgcaccgatatactgactacgatcaagtcattgtcatggcttgacttgatgttgccatttactataattctctccataatcattgcagtaataatttctgtctatgtgccttcttcccgtcacacttttgacgctgaaggtcatcccaatctaatgggagtgtccattcctttgactgttggtatgattgtaatgatgattcccccgatctgcaaagtttcctgggagtctattcacaagtacttctacaggagctatataaggaagcaactagccctctcgttatttttgaattgggtcatcggtcctttgttgatgacagcattggcgtggatggcgctattcgattataaggaataccgtcaaggcattattatgatcggagtagctagatgcattgccatggtgctaatttggaatcagattgctggaggagacaatgatctctgcgtcgtgcttgttattacaaactcgcttttacagatggtattatatgcaccattgcagatattttactgttatgttatttctcatgaccacctgaatacttcaaatagggtattattcgaagaggttgcaaagtctgtcggagtttttctcggcataccactgggaattggcattatcatacgtttgggaagtcttaccatagctggtaaaagtaattatgaaaaatacattttgagatttatttctccatgggcaatgatcggatttcattacactttatttgttatttttattagtagaggttatcaatttatccacgaaattggttctgcaatattgtgctttgtcccattggtgctttacttctttattgcatggtttttgaccttcgcattaatgaggtacttatcaatatctaggagtgatacacaaagagaatgtagctgtgaccaagaactacttttaaagagggtctggggaagaaagtcttgtgaagctagcttttctattacgatgacgcaatgtttcactatggcttcaaataattttgaactatccctggcaattgctatttccttatatggtaacaatagcaagcaagcaatagctgcaacatttgggccgttgctagaagttccaattttattgattttggcaatagtcgcgagaatccttaaaccatattatatatggaacaatagaaattaa

SEQ ID NO: 256 - DNA, 1144 bp, Artificial Sequence
  Pichia pastoris URA6 region
caaatgcaagaggacattagaaatgtgtttggtaagaacatgaagccggaggcatacaaacgattcacagatttgaaggaggaaaacaaactgcatccaccggaagtgccagcagccgtgtatgccaaccttgctctcaaaggcattcctacggatctgagtgggaaatatctgagattcacagacccactattggaacagtaccaaacctagtttggccgatccatgattatgtaatgcatatagtttttgtcgatgctcacccgtttcgagtctgtctcgtatcgtcttacgtataagttcaagcatgtttaccaggtctgttagaaactcctttgtgagggcaggacctattcgtctcggtcccgttgtttctaagagactgtacagccaagcgcagaatggtggcattaaccataagaggattctgatcggacttggtctattggctattggaaccaccctttacgggacaaccaaccctaccaagactcctattgcatttgtggaaccagccacggaaagagcgtttaaggacggagacgtctctgtgatttttgttctcggaggtccaggagctggaaaaggtacccaatgtgccaaactagtgagtaattacggatttgttcacctgtcagctggagacttgttacgtgcagaacagaagagggaggggtctaagtatggagagatgatttcccagtatatcagagatggactgatagtacctcaagaggtcaccattgcgctcttggagcaggccatgaaggaaaacttcgagaaagggaagacacggttcttgattgatggattccctcgtaagatggaccaggccaaaacttttgaggaaaaagtcgcaaagtccaaggtgacacttttctttgattgtcccgaatcagtgctccttgagagattacttaaaagaggacagacaagcggaagagaggatgataatgcggagagtatcaaaaaaagattcaaaacattcgtggaaacttcgatgcctgtggtggactatttcgggaagcaaggacgcgttttgaaggtatcttgtgaccaccctgtggatcaagtgtattcacaggttgtgtcggtgctaaaagagaaggggatctttgccgataacgagacggagaataaataa

SEQ ID NO: 257 - DNA, 600 bp, Artificial Sequence
  Pichia pastoris RPL10 promoter
gttcttcgcttggtcttgtatctccttacactgtatcttcccatttgcgtttaggtggttatcaaaaactaaaaggaaaaatttcagatgtttatctctaaggttttttctttttacagtataacacgtgatgcgtcacgtggtactagattacgtaagttattttggtccggtgggtaagtgggtaagaatagaaagcatgaaggtttacaaaaacgcagtcacgaattattgctacttcgagcttggaaccaccccaaagattatattgtactgatgcactaccttctcgattttgctcctccaagaacctacgaaaaacatttcttgagccttttcaacctagactacacatcaagttatttaaggtatgttccgttaacatgtaagaaaaggagaggatagatcgtttatggggtacgtcgcctgattcaagcgtgaccattcgaagaataggccttcgaaagctgaataaagcaaatgtcagttgcgattggtatgctgacaaattagcataaaaagcaatagactttctaaccacctgtttttttccttttactttatttatattttgccaccgtactaacaagttcagacaaa

SEQ ID NO: 258 - protein, 12 aa, Artificial Sequence
  Connecting peptide
GNGSSSRRAPQT

SEQ ID NO: 259 - protein, 12 aa, Artificial Sequence
  Connecting peptide
GAGNSSRRAPQT

SEQ ID NO: 260 - protein, 13 aa, Artificial Sequence
  Connecting peptide
GAGSNSSRRAPQT

SEQ ID NO: 261 - protein, 13 aa, Artificial Sequence
  Connecting peptide
GNGSNSSRRAPQT

SEQ ID NO: 262 - protein, 12 aa, Artificial Sequence
  Connecting peptide
GAGSSSRRANQT

SEQ ID NO: 263 - protein, 12 aa, Artificial Sequence
  Connecting peptide
GNGSSSRRANQT

SEQ ID NO: 264 - protein, 12 aa, Artificial Sequence
  Connecting peptide
GAGNSSRRANQT

SEQ ID NO: 265 - protein, 13 aa, Artificial Sequence
  Connecting peptide
GAGSNSSRRANQT

SEQ ID NO: 266 - protein, 13 aa, Artificial Sequence
  Connecting peptide
GNGSNSSRRANQT

SEQ ID NO: 267 - protein, 12 aa, Artificial Sequence
  Connecting peptide
GAGSSSRRAPQT

SEQ ID NO: 268 - protein, 6 aa, Artificial Sequence
  Connecting peptide
GGGPRR

SEQ ID NO: 269 - protein, 7 aa, Artificial Sequence
  Connecting peptide
GGGPGAG

SEQ ID NO: 270 - protein, 7 aa, Artificial Sequence
  Connecting peptide
GGGGGKR

SEQ ID NO: 271 - protein, 7 aa, Artificial Sequence
  Connecting peptide
GGGPGKR

SEQ ID NO: 272 - protein, 7 aa, Artificial Sequence
  Connecting peptide
VGLSSGQ

SEQ ID NO: 273 - protein, 7 aa, Artificial Sequence
  Connecting peptide
TGLGSGR

SEQ ID NO: 274 - protein, 7 aa, Artificial Sequence
  Connecting peptide
RRGPGGG

SEQ ID NO: 275 - protein, 7 aa, Artificial Sequence
  Connecting peptide
RRGGGGG

SEQ ID NO: 276 - protein, 9 aa, Artificial Sequence
  Connecting peptide
GGAPGDVKR

SEQ ID NO: 277 - protein, 9 aa, Artificial Sequence
  Connecting peptide
RRAPGDVGG

SEQ ID NO: 278 - protein, 9 aa, Artificial Sequence
  Connecting peptide
GGYPGDVLR

SEQ ID NO: 279 - protein, 9 aa, Artificial Sequence
  Connecting peptide
RRYPGDVGG

SEQ ID NO: 280 - protein, 8 aa, Artificial Sequence
  Connecting peptide
GGHPGDVR

SEQ ID NO: 281 - protein, 9 aa, Artificial Sequence
  Connecting peptide
RRHPGDVGG

SEQ ID NO: 282 - protein, 60 aa, Artificial Sequence; identical to SEQ ID NO: 36
  N-glycosylated proinsulin analogue precursor

SEQ ID NO: 283 - protein, 12 aa, Artificial Sequence
  Connecting peptide
GNGSSSRRAPQT

SEQ ID NO: 284 - protein, 12 aa, Artificial Sequence
  Connecting peptide
GAGNSSRRAPQT

SEQ ID NO: 285 - protein, 13 aa, Artificial Sequence
  Connecting peptide
GAGSNSSRRAPQT

SEQ ID NO: 286 - protein, 13 aa, Artificial Sequence
  Connecting peptide
GNGSNSSRRAPQT

SEQ ID NO: 287 - protein, 12 aa, Artificial Sequence
  Connecting peptide
GAGSSSRRANQT

SEQ ID NO: 288 - protein, 12 aa, Artificial Sequence
  Connecting peptide
GNGSSSRRANQT

SEQ ID NO: 289 - protein, 12 aa, Artificial Sequence
  Connecting peptide
GAGNSSRRANQT

SEQ ID NO: 290 - protein, 13 aa, Artificial Sequence
  Connecting peptide
GAGSNSSRRANQT

SEQ ID NO: 291 - protein, 13 aa, Artificial Sequence
  Connecting peptide
GNGSNSSRRANQT

SEQ ID NO: 292 - protein, 32 aa, Artificial Sequence
  Paucimannose N-glycosylated B-chain peptide
NGTFVNQHLCGSHLVEALYLVCGERGFFYTNK

SEQ ID NO: 293 - protein, 32 aa, Artificial Sequence; identical to SEQ ID NO: 292
  Man5Glc2 N-glycosylated B-chain peptide

SEQ ID NO: 294 - protein, 29 aa, Artificial Sequence
  A2 N-glycosylated B-chain peptide
FVNQHLCGSHLVEALYLVCGERGFFYTNK

SEQ ID NO: 295 - protein, 29 aa, Artificial Sequence
  G2 N-glycosylated B-chain peptide
FVNQHLCGSHLVEALYLVCGERGFFYTNK

SEQ ID NO: 296 - protein, 29 aa, Artificial Sequence
  G0 N-glycosylated B-chain peptide
FVNQHLCGSHLVEALYLVCGERGFFYTNK

SEQ ID NO: 297 - protein, 29 aa, Artificial Sequence
  G-2 N-glycosylated B-chain peptide
FVNQHLCGSHLVEALYLVCGERGFFYTNK

SEQ ID NO: 298 - protein, 30 aa, Artificial Sequence
  Insulin lispro
FVNQHLCGSHLVEALYLVCGERGFFYTKPT

SEQ ID NO: 299 - protein, 30 aa, Artificial Sequence
  Insulin aspart
FVNQHLCGSHLVEALYLVCGERGFFYTDKT

SEQ ID NO: 300 - protein, 30 aa, Artificial Sequence
  Insulin glulisine
FVKQHLCGSHLVEALYLVCGERGFFYTPET

SEQ ID NO: 301 - protein, 29 aa, Artificial Sequence
  Insulin degludec
FVNQHLCGSHLVEALYLVCGERGFFYTPK

SEQ ID NO: 302 - protein, 29 aa, Artificial Sequence
  Insulin detemir
FVNQHLCGSHLVEALYLVCGERGFFYTPK

SEQ ID NO: 303 - protein, 63 aa, Artificial Sequence
  Glycosylated single-chain insulin analogue
FVNQHLCGSHLVEALYLVCGERGFFYTPKTGYGNSSRRANQTGIVEQCCTSICSLYQLENYCN

SEQ ID NO: 304 - protein, 69 aa, Artificial Sequence
  Precursor  single chain insulin analogue with P28N des(B30T)
EEGHHHHHHHHHHEPKFVNQHLCGSHLVEALYLVCGERGFFYTNKAAKGIVEQCCTSICSLYQLENYCN

SEQ ID NO: 305 - protein, 64 aa, Artificial Sequence; identical to SEQ ID NO: 154
  Glycosylated precursor single chain insulin analogue

SEQ ID NO: 306 - DNA, 1430 bp, Artificial Sequence
  Sequence of the 5′-Region used for knock out of YOS9
ccatagcctctgattgatgtaagcaccgacagtacctggctctaacttgttagaggttttggtggtcaagacatatctgttatcacaaataacataatggttatcgggaaagtcattgggatgaacagcaagtgtgttcatgatggcaaattcattacccggagagttgactatcttcaatacatgcacctttggagcatttctctttgtgaatcccagtttttccatggttgtggcaaagtgtagagatgttaagtgcagcgagcaaagacaagtagatagactgtatggtgttctgatgttatagttgtagtgaataatctataaatgccttatttgaaggtttatgtaatagatttacccgtgtgtagcaagtgtactgctaagaggtactataaagttattcatgtggatatattcagtagataataacaaagctacaaggagatcaagaaaccatatgagttgttcgtcacataagagattacgtaatgacaaatcggggaactagtaccaattctgtcttaaagtagtgtctctctaagcataacgacctatttgataactgggctgaactccaagcagcctgatgatgttgacctgacttattcagaagggctattggttttgatttccagatattagcataattagcaatgccggaacaatatacatccaatatttttgaatgaatgaacggttatcaacatttacttctgcctcctcgtctatgacttccttgagttccagcttgttatcggatctgatttttttgattttcttttcttttcttggtagtttgggaattggtgcctgtcgaatttgttcaactattaggttaagacctttctgactagcatcgaagaaggctacattttcgatgtcgttgtgtttgttgatagtcagcttgatatcctgtgcaattggagaacttagtcttttgtaattgaagcagccttcgtccaaacatattctgtaaagatcacttggcaggtctagttgttcaccggtgtgcaatttccattttgagtcaaattctagtgtggccaagttgaacgagttctgagcgaaatcaatagccttcaactgatacgcaaatgtagaccccaagaaaagaaacaacgtgacgaggctttgtagggtagtagccattgtcgaatagttgaggataagtagacggcgagttattctccttgataaatgctatcgcgatggatagtgattacagtgcgataatattatccttttcatccacgtcaaccatggttaacaggccattggacattatgataaaggtcctgctattcctgctctccctatcaagtcttgtgaaagctttggatgattccattgataagaattctgtggtaagtcttttaatttttgttttcacaagatcatgccgtgctaactgggtactatagtatacc

SEQ ID NO: 307 - DNA, 1498 bp, Artificial Sequence
  Sequence of the 3′-Region used for knock out of YOS9
ggttcctattcactgaagacagaatacctcatgacactccaaactttagagtgtataacggagttaatgtgaattaagacaatttatatactcagtaaaataaatactagtacttacgtctttttttagtcagagcactaactctgctggaagggttcttcgtgtaaattggtacagacgctggtaaagtaccactatacgttgtttgacaaataggtagtttgaagctgacatcaagtttcaagtccttaggagtcacattgcgagtttgaatgaccaattgtattaatctcttaatcttgaagtacaatctcttctctttgagactgggtttcaagacagtgacgggattagcaggatcgattttgggtgatgccttatacctttcttgacgtaattgtgacagatctattagcaacttgcttataagttcttgctctttgttggaacggatagcctctatctcatcctcctcaacgaagcttcccggagtccaggagaggaggttgtctagcttgatcttatagtcttcggatccattgacctggacttccttatctgtgttttcaagtttagttgatgtatctgtccccgtatggccattcttagtctcctggtcaacaggtgccggaagctctttttcaattctttttggttcgtccttctgaagttcattatccgtctcatttttagatggtctgctcagtttttctgctatatcaccaagctttctaaaaccagcttgctccagccacctcaggcccttcaattcactggagattgcagatttttcttcgtctattgtaggtgcaaaactgaaatcgttacccttattgtgggtgagccattgacccatcggtaacgcgtaccagttcaaatgaaagaggtttggcaataaatccgtaggtttggtggctgggtgaggttcattgttgtattgaggagaaatcttgttaagcggctgtgaactaatggaagggacatgggggattactttcgtcagattaaaatcgccttcattcactacagcttctctagcatccaagcttgatttattattcagggacgaaaacaatggcgcattaggtgtgatgaatgtagttaaacattctccgttggatgaaacaaaaaatgtggacactttattgaagtcttttgtcatcgattcttcaaactcactggtgtaatcatctaaaacacgagagtcaacgctttctcttagttgtctgtagttgaacaaaaatcttcctgcctctctgatcaataactcaaccatcgacttgtagaacaaatcaatcttgacgtagtcttccgaatctctgttccgttcgtttataagtatcaggcacactaaagttaggtcgtgaaatatggaataaatagtcttgtagtgaccactctttattctgtcgctgatggtaaccagctctgtaggtttgagatccttaccatcaacaagctgatagtatgatccagctatcaaggaaggatcctggac

SEQ ID NO: 308 - DNA, 1699 bp, Artificial Sequence
  Sequence of the 5′-Region used for knock out of ALG3
aaccttcatggaacgattcggatacggaaaaacctgagatagttttaactagagtagatgcaagatttcacgattctaaagaccgagaaggagatgtctgatgtcggtaactactatccggtaaatgatattagcacactatatgctactagcgagtctggaaccaattctactatccattgatgctctattagggatggagaattcaatcaacccctctaattctgatttcagatgttccaacagcgaagtagcccttgacaagttctcaacatcactcatcttagctacattcacgtatgctttgataaaaaactctctacttttgtcaatgagctctagcctagtctctggttctatcgtttcctctttggtctccagattactctctggattagaatctacatccatcttcatatctatgtccatgtccagctcaattttcataccgtcagtattcttagattcgatagcagtatctgatctggtagatccattagttgctgcagcggtattttctttggaatttggagcactttcctgtttctgtttcataaagactcggtagattgcaatgactatatcgtttctgtagaacttgtaaccatgagtccaaaattgggtttcaggcatgtatcctagctcatctaaatatccaaccacatcatccgtgctacatatagtagactcgtagagtgtctgtgaagaaacggctctttttcctgccaaaggaacgtccgatatttgaagggtccatatacgattttccttattaagagcttcaagatgtttcttattaaacaattcaaagtcttttaattcaattgtgttatcaataggatcctcaacgtcctgtttccattcggtggacattctcatcttgtattgttcgatttggttgacttttccagtctggaactcaggactataaggaaactttggagttaaaataacagtataagttgagagccttgcgggcaccatacccgttagagacttcaacgtctccaagatcaactgcagttgagactcttggattctagataccagagacacctgttgtaccatataattaagtgactgggctggcttggatacaggatttcgagaagtgcttcgaattatcagaccgaaggcagttgatattttgtgcctcagccttaatgttccctataacttaaggctatacacagctttatgattaatgaatctgggctgctggtgacgaatttcgtcaatgaccagttgcctacgggcgataattattttttcagttggatgaaagaacggaaaaacccggtcagattcaaaaagaatattgataatctttgtctagcacaactgaaatgcttggaaactctcccaagcatgaatcagacctgagattgtattagacgaaaaaattgtagtatagagttatagacatataggttgtggcaatatcctgtgcaagccaatatctcacagaaataaacgtacacaccagatacaactatttcgaaaagcacactttgagcgcaacagtgattgtcctaacagtataggtttctaaggccccagcagaccatgacggcaaattatttatttcccctcgtatttgccttatctccttttgttctcattcttatcttggctactgtaattatctggataaccctcgatacttcgcttggtttctacctcacaacatatccctacc

SEQ ID NO: 309 - DNA, 1052 bp, Artificial Sequence
  Sequence of the 3′-Region used for knock out of ALG3
atttacaattagtaatattaaggtggtaaaaacattcgtagaattgaaatgaattaatatagtatgacaatggttcatgtctataaatctccggcttcggtaccttctccccaattgaatacattgtcaaaatgaatggttgaactattaggttcgccagtttcgttattaagaaaactgttaaaatcaaattccatatcatcggttccagtgggaggaccagttccatcgccaaaatcctgtaagaatccattgtcagaacctgtaaagtcagtttgagatgaaatttttccggtctttgttgacttggaagcttcgttaaggttaggtgaaacagtttgatcaaccagcggctcccgttttcgtcgcttagtagcagcattattaccaggaatgccgcctgtagagttttgatgtgtcctagctgcaattggagtctgtggagtagtgggagtcgggggctcagtagctttctttgccttctttttagctggctcctttttctttcgtacaggtgcgacattatttggtgtagaccccgcagaagtgttaccagtactatgtgcagtgttttgagtttgtgtaccaggtgaagttccgggagtattcttcgtgaccactgcagagttctggggagggagcattacattcacattaaattttggttcgggcggtgtgtgctctggaattggatcaaagttagaaaaatgcccgcttcccttcttacatgccatgtcatgacgctgtttgttctgtttctcaagcatcattagctctttctgatactcctgtatacctacaattttagaagcacttgattgagactgttgcgattgctggtgttggctctgtgattgtggttgtgctatttgctgatgttgtgaccctggagttggaactagctccggctgctgaatagaagaaggcggagaatgttgcggttgagatgcaggtaaaggctgctgataaacaggaccaggttgcgagaatctaggtgtggtggacgagtgaggagtaccggcggcagaagtagagtgaggcagaggagccat

SEQ ID NO: 310 - DNA, 2559 bp, Artificial Sequence
  DNA encodes LmSTT3A
atgccagctaagaaccaacataagggtggtggtgatggtgatccagacccaacttctactccagctgctgagtccactaaggttacaaacacttccgatggtgctgctgttgattctactttgccaccatccgacgagacttacttgttccactgtagagctgctccatactccaagttgtcctacgctttcaagggtatcatgactgttttgatcttgtgtgctatcagatccgcttaccaagttagattgatctccgttcaaatctacggttacttgatccacgaatttgacccatggttcaactacagagctgctgagtacatgtctactcacggttggtctgcttttttctcctggttcgattacatgtcctggtatccattgggtagaccagttggttctactacttacccaggattgcagttgactgctgttgctatccatagagctttggctgctgctggaatgccaatgtccttgaacaatgtttgtgttttgatgccagcttggtttggtgctatcgctactgctactttggctttgatcgctttcgaagtttccgagtccatttgtatggctgcttgggctgctttgtccttctccattatccctgctcacttgatgagatccatggctggtgagttcgacaacgagtgtattgctgttgctgctatgttgttgactttctactgttgggttagatccttgagaactagatcctcctggccaatcggtgttttgactggtgttgcttacggttacatggctgctgcttggggaggttacatcttcgttttgaacatggttgctatgcacgctggtatctcttctatggttgactgggctagaaacacttacaacccatccttgttgagagcttacactttgttctacgttgttggtactgctatcgctgtttgtgttccaccagttggaatgtctccattcaagtccttggagcagttgggagctttgttggttttggttttcttgtgtggattgcaagtttgtgaggttttgagagctagagctggtgttgaagttagatccagagctaatttcaagatcagagttagagttttctccgttatggctggtgttgctgctttggctatctctgttttggctccaactggttactttggtccattgtctgttagagttagagctttgttcgttgagcacactagaactggtaacccattggttgactccgttgctgaacatcatccagctgacgctttggcttacttgaactacttgcacatcgttcacttgatgtggatctgttccttgccagttcagttgatcttgccatccagaaaccagtacgctgttttgttcgttttggtctactccttcatggcttactacttctccactagaatggttagattgttgatcttggctggtccagttgcttgtttgggagcttctgaagttggtggtactttgatggaatggtgtttccagcaattgttctgggacaacggaatgagaactgctgatatggttgctgctggtgacatgccataccaaaaggacgatcacacttccagaggtgctggtgctagacaaaagcagcagaagcaaaagccaggtcaagtttctgctagaggatcttctacttcctccgaggaaagaccatacagaactttgatcccagttgacttcagaagagatgctcagatgaacagatggtccgctggtaaaactaacgctgctttgatcgttgctttgactatcggagttttgttgccattggctttcgttttccacttgtcctgtatctcttccgcttactcttttgctggtccaagaatcgttttccagactcagttgcacactggtgaacaggttatcgttaaggactacttggaagcttacgagtggttgagagactctactccagaggacgctagagttttggcttggtgggactacggttaccaaatcactggtatcggtaacagaacttccttggctgatggtaacacttggaaccacgagcacattgctactatcggaaagatgttgacttctccagttgctgaagctcactccttggttagacacatggctgactacgttttgatttgggctggtcaatctggtgacttgatgaagtctccacacatggctagaatcggtaactctgtttaccacgacatttgtccagatgacccattgtgtcagcaattcggtttccacagaaacgattactccagaccaactccaatgatgagagcttccttgttgtacaacttgcacgaggctggaaagactaagggtgttaaggttaacccatctttgttccaagaggtttactcctccaagtacggtttggttagaatcttcaaggttatgaacgtttccgctgagtctaagaagtgggttgcagacccagctaacagagtttgtcacccacctggttcttggatttgtcctggtcaatacccacctgctaaagaaatccaagagatgttggctcacagagttccattcgaccaaatggacaagcacaagcagcacaaagaaactcaccacaaggcataa

SEQ ID NO: 311 - DNA, 2322 bp, Artificial Sequence
  DNA encodes LmSTT3B
atgttgttgttgttcttctccttcttgtactgtttgaagaacgcttacggattgagaatgatctccgttcaaatctacggttacttgatccacgaatttgacccatggttcaactacagagctgctgagtacatgtctactcacggttggtctgcttttttctcctggttcgattacatgtcctggtatccattgggtagaccagttggttctactacttacccaggattgcagttgactgctgttgctatccatagagctttggctgctgctggaatgccaatgtccttgaacaatgtttgtgttttgatgccagcttggtttggtgctatcgctactgctactttggctttgatgacttacgaaatgtccggttccggtattgctgctgctattgctgctttcatcttctccatcatcccagctcatttgatgagatccatggctggtgagttcgacaacgagtgtattgctgttgctgctatgttgttgactttctactgttgggttagatccttgagaactagatcctcctggccaatcggtgttttgactggtgttgcttacggttacatggcagctgcttggggaggttacatcttcgttttgaacatggttgctatgcacgctggtatctcttctatggttgactgggctagaaacacttacaacccatccttgttgagagcttacactttgttctacgttgttggtactgctatcgctgtttgtgttccaccagttggaatgtctccattcaagtccttggagcagttgggagctttgttggttttggttttcttgtgtggattgcaagtttgtgaggttttgagagctagagctggtgttgaagttagatccagagctaatttcaagatcagagttagagttttctccgttatggctggtgttgctgctttggctatctctgttttggctccaactggttactttggtccattgtctgttagagttagagctttgttcgttgagcacactagaactggtaacccattggttgactccgttgctgaacacagaatgacttccccaaaggcttacgctttcttcttggacttcacttacccagtttggttgttgggtactgttttgcagttgttgggagcattcatgggttccagaaaagaggctagattgttcatgggattgcattccttggctacttactacttcgctgatagaatgtccagattgatcgttttggctggtccagctgctgctgctatgactgctggaatcttgggattggtttacgaatggtgttgggctcaattgactggatgggcttctcctggtttgtctgctgctggttctggtggaatggatgacttcgacaacaagagaggacaaactcaaatccagtcctccactgctaatagaaacagaggtgttagagcacatgctatcgctgctgttaagtccattaaggctggtgttaacttgttgccattggttttgagagttggtgttgctgttgctattttggctgttactgttggtactccatacgtttcccagttccaggctagatgtattcaatccgcttactcctttgctggtccaagaatcgttttccaggctcagttgcacactggtgaacaggttatcgttaaggactacttggaagcttacgagtggttgagagactctactccagaggacgctagagttttggcttggtgggactacggttaccaaatcactggtatcggtaacagaacttccttggctgatggtaacacttggaaccacgagcacattgctactatcggaaagatgttgacttctccagttgctgaagctcactccttggttagacacatggctgactacgttttgatttgggctggtcaatctggtgacttgatgaagtctccacacatggctagaatcggtaactctgtttaccacgacatttgtccagatgacccattgtgtcagcaattcggtttccacagaaacgattactccagaccaactccaatgatgagagcttccttgttgtacaacttgcacgaggctggtaaaactaagggtgttaaggttaacccatctttgttccaagaggtttactcctccaagtacggtttggttagaatcttcaaggttatgaacgtttccgctgagtctaagaagtgggttgcagacccagctaacagagtttgtcacccacctggttcttggatttgtcctggtcaatacccacctgctaaagaaatccaagagatgttggctcacagagttccattcgaccaaatggacaagcacaagcagcacaaagaaactcaccacaaggcataa

SEQ ID NO: 312 - DNA, 2004 bp, Artificial Sequence
  Pichia pastoris ATT1 5′ region
ggccgggactacatgaggccgattcttcaagccagggaaattaattgcttgaaccggaaaatcattaaggcaggcaacgaaaaatccaactccttggttgaattgactcaaaagtttatcttacggagaaaagctaaagacatcaatacgaatttccttccgccaaaaactgaactgatactgatggttccaatgactgaattacaacaggagctatacaaggatataattgaaactaaccaagccaagcttggcttgatcaacgacagaaacttttttcttcaaaaaattttgattcttcgtaaaatatgcaattcaccctccctgctgaaagacgaacctgattttgccagatacaatctcggcaatagattcaatagcggtaagatcaagctaacagtactgcttttacgaaagctgtttgaaaccaccaatgagaagtgtgtgattgtttcaaacttcactaaaactttggacgtacttcagctaatcatagagcacaacaattggaaataccaccgactagatggttcgagtaaaggacgggacaaaatcgtacgagattttaacgagtcgcctcaaaaagatcgattcatcatgttgctttcttccaaggcagggggagtggggctcaacttaattggagcctcacgcttaattctttttgataacgactggaatcccagtgttgacattcaagcaatggctagagtgcatcgagacgggcagaaaaggcacacctttatctatcgtttgtatacgaaaggcacaattgacgaaaagatcctacaaaggcaattgatgaaacaaaatctgagcgacaaattcctggatgataatgatagcagcaaggatgatgtgtttaacgactacgatctcaaagatttgtttactgtagatcttgacacgaattgtagtacacacgatttgatggaatgtttatgtaatgggcggctgagagatccgactcccgtcttggaagcagaagaatgcaagacaaaaccgttggaggccgttgacgacacggatgatggttggatgtcagctctggatttcaaacagttatcacaaaaagaggagacaggtgctgtgtcaacaatgcgtcaatgtctgctcggatatcaacacattgatccaaagattttggaaccaacagaacctgtaggggacgatttggtattggcaaacatcctcgcggagtcctcaggcttggctaaatctgcattgtcatctgaaaagaaacccaagaaaccagtggtgaactttatctttgtgtcaggccaagactaagctggaagaacggaactttaatcgaaggaaaaattaaatgtcaaagtgggtcgatcaggagataatccatgcttcacgtgatttttcttaataaacgccggaaaaactttcttttttgtgaccaaaattatccgatctgaaaaaaaattacgcatgcgtgaagtaggatgagagacttactgttgaactttgtgagacgaggggaaaaggaatatcctgatcgtaaacaaaaaagttttccagcccaatcgggaacatctgcgaagtgttggaattcaacccctctttcgaaaatgttccattttacccaaaattattgttattaaataatacatgtgttactagcaaagtctgcgctttccatgtctcagattcggcagataacaaagttgacacgttcttgcgagatacgcatgaatcttttggctgctttttgtgaaagagaaatggtgccatatattgcagacgcccctgaaagattagtgtgcggctgagtcttttttttttctcaaccagctttttctttttattgggtaccatcgcgcacgcaggactcatgctccattagacttctgaaccacctgacttaatattcatggacggacgcttttatccttaaattgttcatccattcctcaatttttccgtttgccctccctgtactattaaattacaaaagctgatctttttcaagtgtttctctttgaatcgctc

SEQ ID NO: 313 - DNA, 1854 bp, Artificial Sequence
  Pichia pastoris ATT1 3′ region
ggaccctgaagacgaagacatgtctgccttagagtttaccgcagttcgattccccaacttttcagctacgacaacagccccgcctcctactccagtcaattgcaacagtcctgaaaacatcaagacctccactgtggacgattttttgaaagctactcaagatccaaataacaaagagatactcaacgacatttacagtttgatttttgatgactccatggatcctatgagcttcggaagtatggaaccaagaaacgatttggaagttccggacactataatggattaatttgcagcgggcctgtttgtatagtctttgattgtgtataatagaattactacgcgtatatcccgatctggaagtaacatggaagtttcccattttcgcgcagtctcctactcgtatcctccccaccccttaccgatgacgcaaaaggtcactagataagcatagcatagtttcatcccttgctctttccttgtaccaacagatcatggctgggaatctcaaggatattctatccttgtcgaggaagacagcaaggaatctgaagcaggctctggatgagcttgcggagcaggtgatcaaccaccaacggagacgaccagctctggtccgagttcctatcaacaacaaccttaggcgcaagagccagcagtcctttttgaatcgcaggtcattccatctttggaccagcaagtacaacccatacttttggaggggaggcagaagcaacgttctggaccagcttaaccgtgaagctttaaggtacagatcgtcttttgcgaaacccggattttatccaagtgggctgtatcagtcaactttccctcaaagaggtagtaggatgttttccacctgcgcctactcatgtcagcaggaggcagtcaaaaacttgacttccgctgttcgtgctttgttacaaagtggtgctaatttcggcagtcaaatgaaacaaatgaaacactgttcgcaaaagaagaagcacttctctaaattttctaagaggcttacttcttccactgccgctgggtctggcaagaatgctgaacaagctccttctggtttggccgaaggatccgctgttgtttttagccttgaacgtcaaagtcacaatactgagttggaaggaatcttggatcaagaaacttcttccattctcgaggaagaaatggttcaacatgagcgtcacctggctattattagagaagaaatccagagaattagtgagaatctaggatcattaccattaatcatgtctggtcacaagattgaggtatttttccccaattgtgacactgttaaatgtgagcaactgatgagagatttggctattacgaaaggggttgtgaggcgtcatgattctactgctgagcattcaagctccaggtcatttgttccagaagattgcttgtattcctcagggtcaagttcaccgaatcctttatcctcaacttcttcgaaatcatttgatagagtctcattggactacatttcctctcggtctacatctgatcaaaccactggttctgagtacacatctctgtctcaacaatatcacctggttagcaattacaaccctgtactatcctcagccccgggttcttcgagggtcttggagctgaatactcccgagtccactatggaaggcagtacagatctggagtatttaacgcgagacgatgtgttgctgttaaatgtctaatctagacctatccttcattctatatagcttagttgagttttacgtaagccctagtttttgttaattcttatcgatttatggttagtgtaccactcaactcacgatgatatatcccaggagctgtttgtgcattataactaccaatcct

SEQ ID NO: 314 - DNA, 1389 bp, Artificial Sequence
  DNA encodes murine endomannosidase codon- optimized
atggctaagtttagaagaagaacctgtattttgttgtccttgtttatcctttttattttctccttgatgatgggattgaagatgctttggcctaacgctgcctcttttggtccacctttcggattggatttgcttccagaacttcatcctttgaacgcacactcaggtaataaggctgattttcagagaagtgacagaattaacatggaaactaacacaaaggctttgaaaggtgccggaatgactgttcttcctgccaaagcatccgaggtcaaccttgaagagttgccacctcttaactactttttgcatgctttctactactcatggtacggtaacccacaattcgatggaaagtacatccattggaatcacccagttttggaacattgggaccctagaatcgctaaaaattacccacagggtcaacactctccacctgatgacattggttcttccttctaccctgaattgggatcttattcaagtagagatccatccgttattgagactcatatgaagcaaatgagatccgcctccatcggtgtcttggcactttcatggtacccacctgacagtagagatgacaacggagaagccacagatcacttggttcctaccattcttgacaaggcacataagtacaacttgaaggtcactttccacatcgagccatattctaatagagatgaccagaacatgcaccaaaacatcaagtacatcatcgataagtacggtaaccatcctgctttctacagatataagaccagaactggacactctttgccaatgttctacgtttatgactcctacattacaaaacctaccatctgggctaacttgcttactccatcaggtagtcagtcggttagatcctccccttatgatggattgtttattgccttgcttgtcgaagagaagcataagaacgatatcttgcagtctggtttcgacggaatctacacatattttgctaccaacggtttcacttacggatcaagtcaccaaaattggaacaatttgaagtccttctgtgaaaagaacaatcttatgttcatcccatcagttggtcctggatatattgatacaagtatcagaccatggaacactcaaaacacaagaaacagagttaacggtaaatactacgaggtcggattgtctgcagctcttcagactcatccttccttgatttcaatcacaagttttaacgaatggcacgagggtactcaaattgaaaaggctgttccaaaaagaaccgccaatactatctacttggattatagaccacataagccttcattgtaccttgagttgaccagaaaatggtctgaaaagttctccaaagagagaatgacttatgcattggaccaacagcaaccagcttcctaa

SEQ ID NO: 315 - protein, 260 aa, Artificial Sequence
  P. pastoris AOX1 transcription termination sequence
TCAAGAGGATGTCAGAATGCCATTTGCCTGAGAGATGCAGGCTTCATTTTGATACTTTTTTATTTGTAACCTATATAGTATAGGATTTTTTTTGTCATTTTGTTTCTTCTCGTACGAGCTTGCTCCTGATCAGCCTATCTCGCAGCTGATGAATATCTTGTGGTAGGGGTTTGGGAAAATCATTCGAGTTTGATGTTTTTCTTGGTATTTCCCACTCCTCTTCAGAGTACAGAAGATTAAGTGAGACGTTCGTTTGTGCA

SEQ ID NO: 316 - protein, 21 aa, Artificial Sequence
  A-chain analog
GIVEQCCTSNCSLYQLENYCG

SEQ ID NO: 317 - protein, 21 aa, Artificial Sequence
  A-chain analog
GIVEQCCNSSCSLYQLENYCG

SEQ ID NO: 318 - protein, 21 aa, Artificial Sequence
  A-chain analog
GIVEQCCNRSCSLYQLENYCG

SEQ ID NO: 319 - protein, 35 aa, Artificial Sequence
  B-chain analog
NTTFVNQHLCGSHLVEALYLVCGERGFFYTPKTRR

SEQ ID NO: 320 - protein, 35 aa, Artificial Sequence
  B-chain analog
NTTFVNQHLCGSHLVEALYLVCGERGFFYTNKTRR

SEQ ID NO: 321 - protein, 32 aa, Artificial Sequence
  B-chain analog
FVNETLCGSHLVEALYLVCGERGFFYTPKTRR

SEQ ID NO: 322 - protein, 32 aa, Artificial Sequence; identical to SEQ ID NO: 207
  B-chain analog

SEQ ID NO: 323 - protein, 32 aa, Artificial Sequence
  B-chain analog
FVNQHLCGSHLVEALYLVCGERGFNFTPKTRR

SEQ ID NO: 324 - protein, 32 aa, Artificial Sequence
  B-chain analog
FVNQTLCGSHLVEALYLVCGERGFFYTNKTRR

SEQ ID NO: 325 - protein, 32 aa, Artificial Sequence
  B-chain analog
FVNETLCGSHLVEALYLVCGERGFFYTNKTRR

SEQ ID NO: 326 - protein, 32 aa, Artificial Sequence; identical to SEQ ID NO: 209
  B-chain analog

SEQ ID NO: 327 - protein, 32 aa, Artificial Sequence; identical to SEQ ID NO: 208
  B-chain analog

SEQ ID NO: 328 - protein, 33 aa, Artificial Sequence
  B-chain analog
NGTFVNQHLCGSHLVEALYLVCGERGFFYTDKT

SEQ ID NO: 329 - protein, 32 aa, Artificial Sequence
  B-chain analog
NGTFVNQHLCGSHLVEALYLVCGERGFFYTDK

SEQ ID NO: 330 - protein, 33 aa, Artificial Sequence
  B-chain analog
NGTFVNETLCGSHLVEALYLVCGERGFFYTDKT

SEQ ID NO: 331 - protein, 32 aa, Artificial Sequence
  B-chain analog
NGTFVNETLCGSHLVEALYLVCGERGFFYTDK

SEQ ID NO: 332 - protein, 30 aa, Artificial Sequence
  B-chain analog
FVNETLCGSHLVEALYLVCGERGFNFTDKT

SEQ ID NO: 333 - protein, 29 aa, Artificial Sequence
  B-chain analog
FVNETLCGSHLVEALYLVCGERGFNFTDK

SEQ ID NO: 334 - protein, 33 aa, Artificial Sequence
  B-chain analog
NGTFVNQHLCGSHLVEALYLVCGERGFFYTKPT

SEQ ID NO: 335 - protein, 33 aa, Artificial Sequence
  B-chain analog
NGTFVKQHLCGSHLVEALYLVCGERGFFYTPET

SEQ ID NO: 336 - protein, 33 aa, Artificial Sequence
  B-chain analog
NGTFVNETLCGSHLVEALYLVCGERGFNYTDKT

SEQ ID NO: 337 - protein, 32 aa, Artificial Sequence
  B-chain analog
NGTFVNETLCGSHLVEALYLVCGERGFNYTDK